(19) United States
(12) Patent Application Publication
(10) Pub. No.: US 2017/0244049 A1
(43) Pub. Date: Aug. 24, 2017

(54) ORGANIC LIGHT-EMITTING DIODE MATERIALS

(71) Applicant: President and Fellows of Harvard College, Cambridge, MA (US)

(72) Inventors: Alan Aspuru-Guzik, Cambridge, MA (US); Rafael Gomez-Bombarelli, Cambridge, MA (US); Timothy D. Hirzel, Quincy, MA (US); Jorge Aguilera-Iparraguirre, Roslindale, MA (US); Ryan P. Adams, Somerville, MA (US); Dougal Maclaurin, Brookline, MA (US); David K. Duvenaud, Cambridge, MA (US)

(73) Assignee: President and Fellows of Harvard College, Cambridge, MA (US)

(21) Appl. No.: 15/310,234

(22) PCT Filed: May 13, 2015
(86) PCT No.: PCT/US2015/030598

§ 371 Date: Nov. 10, 2016

Related U.S. Application Data
(60) Provisional application No. 61/996,836, filed on May 14, 2014.
(60) Provisional application No. 61/997,579, filed on Jun. 5, 2014.
(60) Provisional application No. 62/028,045, filed on Jul. 23, 2014.
(60) Provisional application No. 62/033,869, filed on Aug. 6, 2014.
(60) Provisional application No. 62/048,497, filed on Sep. 10, 2014.
(60) Provisional application No. 62/061,369, filed on Oct. 8, 2014.
(60) Provisional application No. 62/061,460, filed on Oct. 8, 2014.
(60) Provisional application No. 62/075,490, filed on Nov. 5, 2014.
(60) Provisional application No. 62/093,097, filed on Dec. 17, 2014.
(60) Provisional application No. 62/117,045, filed on Feb. 17, 2015.
(60) Provisional application No. 62/139,336, filed on Mar. 27, 2015.
(60) Provisional application No. 62/155,764, filed on May 1, 2015.

(51) Int. Cl. H01L 51/00; C07D 403/14; C07D 403/10; C07D 265/38; C07D 401/14; C07D 209/88; C07D 251/24; C07D 487/04; C07D 413/10; C07C 225/22; C07D 413/14; C07D 401/10; C07D 209/86; C07D 471/04; C07D 409/14; C09K 11/06

(52) U.S. Cl. CPC H01L 51/0072; C09K 11/06; C07D 403/14; H01L 51/0067; C07D 403/10; C07D 265/38; H01L 51/0071; C07D 401/14; H01L 51/0061; C07D 209/88; C07D 251/24; C07D 487/04; C07D 413/10; C07C 225/22; H01L 51/0059; C07D 413/14; C07D 401/10; C07D 209/86; C07D 471/04; C07D 409/14; H01L 51/0074; C09K 2211/1059; C09K 2211/1022; C09K 2211/1007; C09K 2211/1014; C09K 2211/1074; C09K 2211/1033; C09K 2211/1029; C09K 2211/1044; C09K 2211/1048; C09K 2211/1062; C09K 2211/1081; H01L 51/5012

(57) ABSTRACT

Described herein are molecules for use in organic light emitting diodes. Example molecules comprise at least one acceptor moiety A, at least one donor moiety D, and optionally one or more bridge moieties B. Each moiety A is covalently attached to either the moiety B or the moiety D, each moiety D is covalently attached to either the moiety B or the moiety A, and each moiety B is covalently attached to at least one moiety A and at least one moiety D. Values and preferred values of moieties A, D, and B are defined herein.

### RELATED APPLICATIONS

This application claims the benefit of U.S. Provisional Application No. 61/996,836, filed on May 14, 2014; U.S. Provisional Application No. 61/997,579, filed on Jun. 5, 2014; U.S. Provisional Application No. 62/028,045, filed on Jul. 23, 2014; U.S. Provisional Application No. 62/033,869, filed on Aug. 6, 2014; U.S. Provisional Application No. 62/048,497, filed on Sep. 10, 2014; U.S. Provisional Application No. 62/061,369, filed on Oct. 8, 2014; U.S. Provisional Application No. 62/061,460, filed on Oct. 8, 2014; U.S. Provisional Application No. 62/075,490, filed on Nov. 5, 2014; U.S. Provisional Application No. 62/093,097, filed on Dec. 17, 2014; U.S. Provisional Application No. 62/117,045, filed on Feb. 17, 2015; U.S. Provisional Application No. 62/139,336, filed on Mar. 27, 2015; and U.S. Provisional Application No. 62/155,764, filed on May 1, 2015. The entire teachings of each application above are incorporated herein by reference.

### BACKGROUND OF THE INVENTION

An organic light emitting diode (OLED) is a light-emitting diode (LED) in which a film of organic compounds is placed between two conductors and emits light in response to excitation, such as an electric current. OLEDs are useful in displays such as television screen, computer monitors, mobile phones, and tablets. A problem inherent in OLED displays is the limited lifetime of the organic materials. OLEDs which emit blue light, in particular, degrade at a significantly increased rate as compared to green or red OLEDs.

OLED materials rely on the radiative decay of molecular excited states (excitons) generated by recombination of electrons and holes in a host transport material. The nature of excitation results in interactions between electrons and holes that split the excited states into bright singlets (with a total spin of 0) and dark triplets (with a total spin of 1). Since the recombination of electrons and holes affords a statistical mixture of four spin states (one singlet and three triplet sublevels), conventional OLEDs have a maximum theoretical efficiency of 25%.

To date, OLED material design has focused on harvesting the remaining energy from the normally dark triplets into an emissive state. Recent work to create efficient phosphors, which emit light from the normally dark triplet state, have resulted in green and red OLEDs. Other colors, such as blue, however, require higher energy excited states which enhance the degradation process of the OLED.

The fundamental limiting factor to the triplet-singlet transition rate is a value of the parameter |H_{fi}/Δ|², where H_{fi} is the coupling energy due to hyperfine or spin-orbit interactions, and A is the energetic splitting between singlet and triplet states. Traditional phosphorescent OLEDs rely on the mixing of singlet and triplet states due to spin-orbital (SO) interaction, increasing H_{fi} and affording a lowest emissive state shared between a heavy metal atom and an organic ligand. This results in energy harvesting from all higher singlet and triplet states, followed by phosphorescence (relatively short-lived emission from the excited triplet). The shortened triplet lifetime reduces triplet exciton annihilation by charges and other excitons. Recent work by others suggests that the limit to the performance of phosphorescent materials has been reached.

### SUMMARY OF THE INVENTION

Thus, a need exists for OLEDs which can reach higher excitation states without rapid degradation. It has now been discovered that thermally activated delayed fluorescence (TADF), which relies on minimization of A as opposed to maximization of Ha, can transfer population between singlet levels and triplet sublevels in a relevant timescale, such as, for example, 110 μs. The compounds described herein are capable of fluorescing or phosphorescing at higher energy excitation states than compounds previously described.

Accordingly, in one embodiment, the present invention is a molecule represented by structural formula (XII):

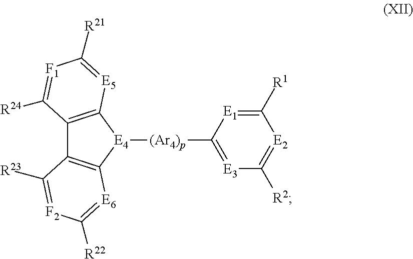

In structural formula (XII) of the present invention:

E₁, E₂, E₃, E₄, E₅, and E₆, are, each independently, CH or N.

R¹ and R² are, each independently, H, a C₁-C₆ alkyl, a C₆-C₁₈ aryl, or a (5-20) atom heteroaryl.

R²¹, R²², R²³, and R²⁴ are, each independently, H, or a C₁-C₃ alkyl.

F₁ and F₂ are, each independently, CR′ or N, wherein R′ is H, a C₁-C₆ alkyl, a C₆-C₁₈ aryl, or —(Ar₅)_{q}-G.

Ar₄ or Ar₅ are, each independently, phenyl optionally substituted with one to four C₁-C₃ alkyls.

p is 0, 1, or 2.

q is 0 or 1.

G is H, or a moiety represented by one of the following structural formula:

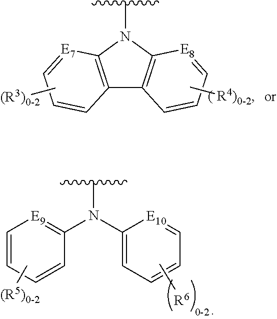

wherein E₇, E₈, E₉, and E₁₀ are, each independently, CH or N, and R³, R⁴, R⁵, and R⁶ are, each independently, a C₁-C₃ alkyl, a C₆-C₁₈ aryl, a halo, or —CN.

In structural formula (XII) of the present invention, when E₁, E₂, and E₃ are each N, and F₁ and F₂ are each CR′, then each R′ is not the moiety represented by the structural formula:

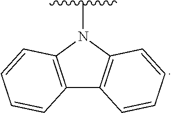 [SMILES: *n1c2ccccc2c2ccccc21]

In another embodiment, the present invention is the present invention is a molecule comprising at least one acceptor moiety A, at least one donor moiety D, and optionally, a bridge moiety B. Each moiety A is bonded either to moiety B or moiety D, each moiety B is bonded either to moiety A, moiety D, or a second moiety B, and each moiety D is bonded either to moiety A or moiety B. The moiety A, for each occurrence independently, is selected from List A1, List A2, List A3, or any combination thereof. The moiety D, for each occurrence independently, is selected from List D1, List D2, List D3, or any combination thereof. The moiety B, for each occurrence independently, is selected from List B1, B2, or both. The molecule is represented by any one of the structural formulas in Tables 1-14, wherein the carbon or heteroatom denoted by (*) in the structural formulas represented in Tables 1-14 is unsubstituted or substituted by a C₁-C₆ alkyl, —OH, —CN, a halo, a C₆-C₁₂ aryl, a 5-20 atom heteroaryl, —N(R¹⁹)₂, or —N(R²⁰)₂. Each R¹⁹, independently, is H, a C₁-C₆ alkyl, or a C₅-C₁₂ cycloalkyl, and each R²⁰, independently, is H or a C₆-C₁₈ aryl. Provided, the molecule is not represented by the structural formulas B4, J68, J79, K39, K55, K57, K100, K177, or N6 in Tables 1-14.

In another embodiment, the present invention is a molecule represented by structural formulas (II)-(XI):

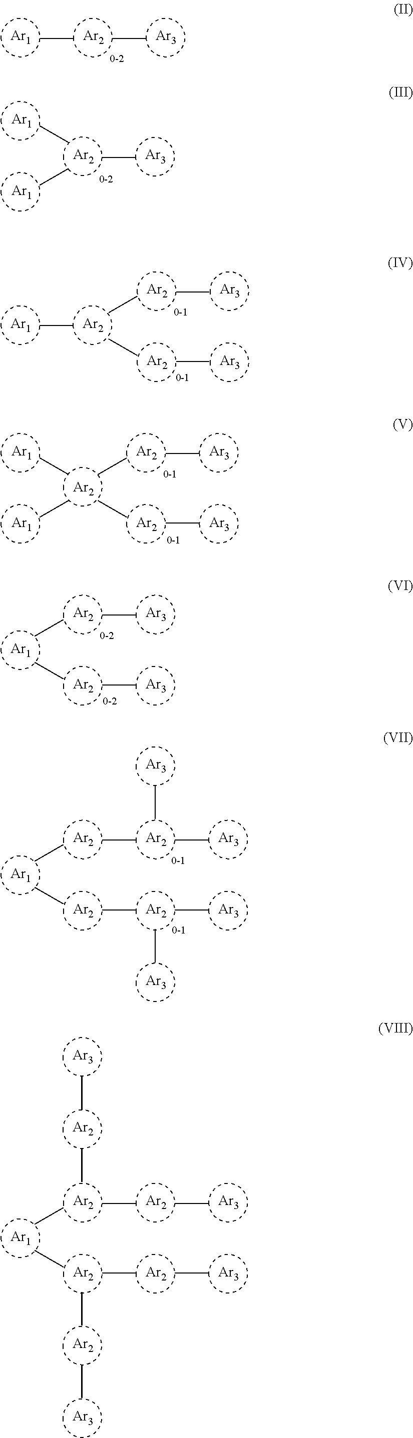

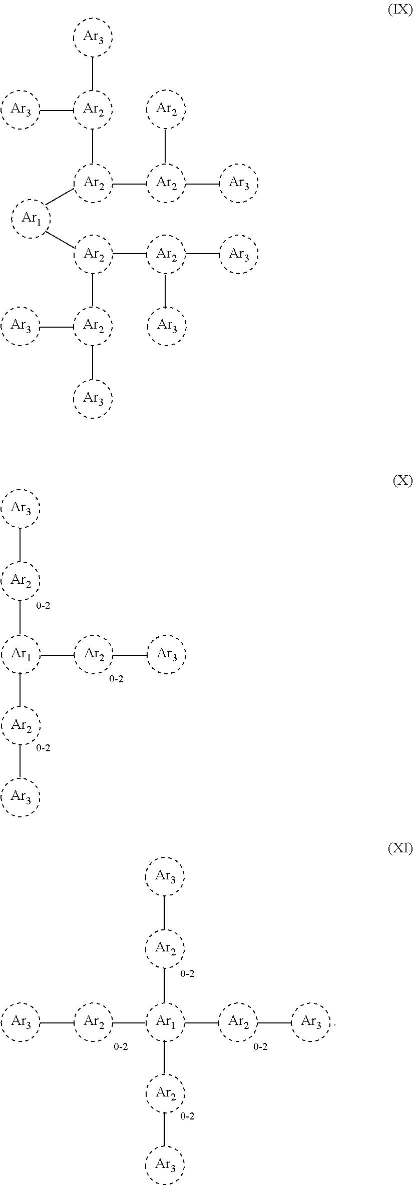

In structural formulas (II)-(XI), Ar₁ and Ar₃, for each occurrence independently, are selected from List M1, with the understanding that Ar₁ and Ar₃ are different. Ar₂ is, for each occurrence independently, selected List M2. The molecule is represented by any one of the structural formulas in Tables 1-14, wherein the carbon or heteroatom denoted by (*) in the structural formulas represented in Tables 1-14 is unsubstituted or substituted by a C₁-C₆ alkyl, —OH, —CN, a halo, a C₆-C₁₂ aryl, a 5-20 atom heteroaryl, —N(R¹⁹)₂, or —N(R²⁰)₂. Each R¹⁹, independently, is H or a C₁-C₆ alkyl, or a C₅-C₁₂ cycloalkyl, and each R²⁰, independently, is H or a C₆-C₁₈ aryl. Provided, the molecule is not represented by the structural formulas B4, J68, J79, K39, K55, K57, K100, K177, or N6 in Tables 1-14.

In another embodiment, the present invention is an organic light-emitting device comprising a first electrode, a second electrode, and an organic layer between the first electrode and the second electrode. The organic layer comprises at least one light-emitting molecule selected from structural formulas (II)-(XII) or the structural formulas represented in Tables 1-14.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a scatter plot illustrating the relationship between the brightness of an OLED as compared to the time of decay after excitation. The plot illustrates that brightness of the OLED decreases as the time of decay increases.

FIGS. 2-30C are synthetic schemes (Schemes 1-31) illustrating synthesis of example embodiments of the present invention.

FIGS. 31A-44 are Tables 1-14, which illustrate structural formulas of example embodiments of molecules useful in the present invention.

FIGS. 45A-45B is Table 15, which illustrates structural formulas of example embodiments of the present invention.

FIG. 46A-46H is Table 16, which illustrates structural formulas of certain compounds.

FIGS. 47A-47CCC is Table 17, which illustrates emission data for the example compounds in Tables 1-14. The data includes calculated HOMO and LUMO values, vertical absorption, emission wavelength, the singlet-triplet energy gap, and the S1 to S0 oscillator strength.

FIG. 48A-48B is Table 18, which illustrates structural formulas of certain compounds.

### DETAILED DESCRIPTION OF THE INVENTION

A description of example embodiments of the invention follows.

### Glossary

The term “alkyl,” as used herein, refers to a saturated aliphatic branched or straight-chain monovalent hydrocarbon radical having the specified number of carbon atoms. Thus, “C₁-C₆ alkyl” means a radical having from 1-6 carbon atoms in a linear or branched arrangement. Examples of “C₁-C₆ alkyl” include, n-propyl, i-propyl, n-butyl, i-butyl, sec-butyl, t-butyl, n-pentyl, n-hexyl, 2-methylbutyl, 2-methylpentyl, 2-ethylbutyl, 3-methylpentyl, and 4-methylpentyl. An alkyl can be optionally substituted with halogen, —OH, C₁-C₆ alkyl, C₁-C₆ alkoxy, —NO₂, —CN, and —N(R′)(R²) wherein R¹ and R² are each independently selected from —H and C₁-C₃ alkyl.

The term “alkenyl,” as used herein, refers to a straight-chain or branched alkyl group having one or more carbon-carbon double bonds. Thus, “C₂-C₆ alkenyl” means a radical having 2-6 carbon atoms in a linear or branched arrangement having one or more double bonds. Examples of “C₂-C₆ alkenyl” include ethenyl, propenyl, butenyl, pentenyl, hexenyl, butadienyl, pentadienyl, and hexadienyl. An alkenyl can be optionally substituted with the substituents listed above with respect to alkyl.

The term “alkynyl,” as used herein, refers to a straight-chain or branched alkyl group having one or more carbon-carbon triple bonds. Thus, “C₂-C₆ alkynyl” means a radical having 2-6 carbon atoms in a linear or branched arrangement having one or more triple bonds. Examples of C₂-C₆ “alkynyl” include ethynyl, propynyl, butynyl, pentynyl, and hexynyl. An alkynyl can be optionally substituted with the substituents listed above with respect to alkyl.

The term “cycloalkyl,” as used herein, refers to a saturated monocyclic or fused polycyclic ring system containing from 3-12 carbon ring atoms. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2]bicyclooctane, decahydronaphthalene and adamantane. A cycloalkyl can be optionally substituted with the substituents listed above with respect to alkyl.

The term “amino,” as used herein, means an “—NH₂,” an “NHRₚ” or an “NRₚR_{q},” group, wherein Rₚ and R_{q} can be alkyl, alkenyl, alkynyl, alkoxy, cycloalkyl, aryl, and heteroaryl. Amino may be primary (NH₂), secondary (NHRₚ) or tertiary (NRₚR_{q}).

The term “alkylamino,” as used herein, refers to an “NHRₚ,” or an “NRₚR_{q}” group, wherein Rₚ and R_{q} can be alkyl, alkenyl, alkynyl, alkoxy, or cycloalkyl. The term “dialkylamino,” as used herein, refers to an “NRₚR_{q}” group, wherein Rₚ and R_{q} can be alkyl, alkenyl, alkynyl, alkoxy, or cycloalkyl.

The term “alkoxy”, as used herein, refers to an “alkyl-O—” group, wherein alkyl is defined above. Examples of alkoxy group include methoxy or ethoxy groups. The “alkyl” portion of alkoxy can be optionally substituted as described above with respect to alkyl.

The term “aryl,” as used herein, refers to an aromatic monocyclic or polycyclic ring system consisting of carbon atoms. Thus, “C₆-C₁₈ aryl” is a monocylic or polycyclic ring system containing from 6 to 18 carbon atoms. Examples of aryl groups include phenyl, indenyl, naphthyl, azulenyl, heptalenyl, biphenyl, indacenyl, acenaphthylenyl, fluorenyl, phenalenyl, phenanthrenyl, anthracenyl, cyclopentacyclooctenyl or benzocyclooctenyl. An aryl can be optionally substituted with halogen, —OH, C₁-C₆ alkyl, C₂-C₆ alkenyl, C₂-C₆ alkynyl, C₁-C₆ haloalkyl, C₁-C₆ alkoxy, C₆-C₁₈ aryl, C₆-C₁₈ haloaryl, (5-20 atom) heteroaryl, —C(O)C₁-C₃ haloalkyl, —S(O)₂—, —NO₂, —CN, and oxo.

The terms “halogen,” or “halo,” as used herein, refer to fluorine, chlorine, bromine, or iodine.

The term “heteroaryl,” as used herein, refers a monocyclic or fused polycyclic aromatic ring containing one or more heteroatoms, such as oxygen, nitrogen, or sulfur. For example, a heteroaryl can be a “5-20 atom heteroaryl,” which means a 5 to 20 membered monocyclic or fused polycyclic aromatic ring containing at least one heteroatom. Examples of heteroaryl groups include pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl. A heteroaryl can be optionally substituted with the same substituents listed above with respect to aryl.

In other embodiments, a “5-20 member heteroaryl” refers to a fused polycyclic ring system wherein aromatic rings are fused to a heterocycle. Examples of these heteroaryls include:

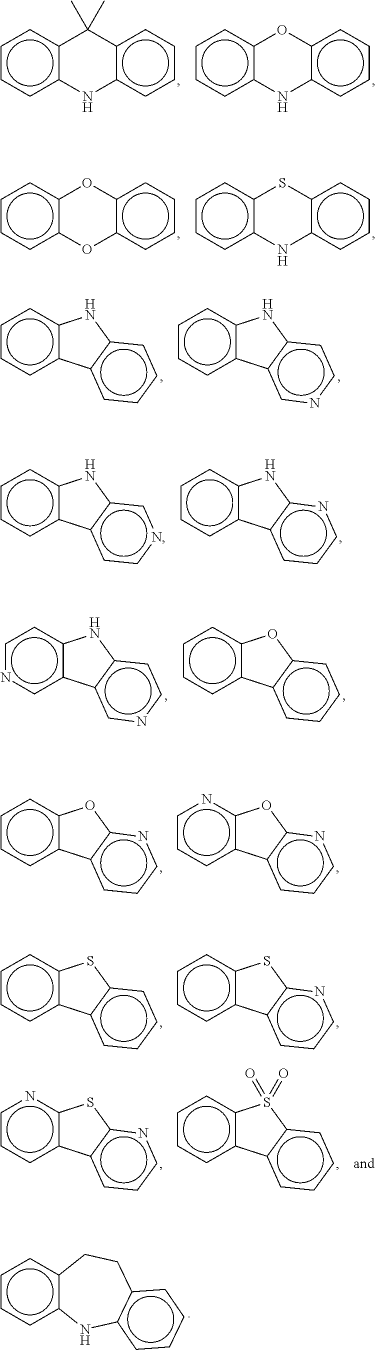

The term “haloalkyl,” as used herein, includes an alkyl substituted with one or more of F, Cl, Br, or I, wherein alkyl is defined above. The “alkyl” portion of haloalkyl can be optionally substituted as described above with respect to alkyl.

The term “haloaryl,” as used herein, includes an aryl substituted with one or more of F, Cl, Br, or I, wherein aryl is defined above. The “aryl” portion of haloaryl can be optionally substituted as described above with respect to aryl.

The term “oxo,” as used herein, refers to ═O.

The term “nitro,” as used herein, refers to —NO₂.

The term “symmetrical molecule,” as used herein, refers to molecules that are group symmetric or synthetic symmetric. The term “group symmetric,” as used herein, refers to molecules that have symmetry according to the group theory of molecular symmetry. The term “synthetic symmetric,” as used herein, refers to molecules that are selected such that no regioselective synthetic strategy is required.

The term “donor,” as used herein, refers to a molecular fragment that can be used in organic light emitting diodes and is likely to donate electrons from its highest occupied molecular orbital to an acceptor upon excitation. In an example embodiment, donors have an ionization potential greater than or equal to −6.5 eV.

The term “acceptor,” as used herein, refers to a molecular fragment that can be used in organic light emitting diodes and is likely to accept electrons into its lowest unoccupied molecular orbital from a donor that has been subject to excitation. In an example embodiment, acceptors have an electron affinity less than or equal to −0.5 eV.

The term “bridge,” as used herein, refers to a x-conjugated molecular fragment that can be included in a molecule which is covalently linked between acceptor and donor moieties. The bridge can, for example, be further conjugated to the acceptor moiety, the donor moiety, or both. Without being bound to any particular theory, it is believed that the bridge moiety can sterically restrict the acceptor and donor moieties into a specific configuration, thereby preventing the overlap between the conjugated r system of donor and acceptor moieties. Examples of suitable bridge moieties include phenyl, ethenyl, and ethynyl.

The term “multivalent,” as used herein, refers to a molecular fragment that is connected to at least two other molecular fragments. For example, a bridge moiety, is multivalent.

“ [symbol] ” as used h pi F ers to a point of attachment between two atoms.

### Principles of OLED

OLEDs are typically composed of a layer of organic materials or compounds between two electrodes, an anode and a cathode. The organic molecules are electrically conductive as a result of delocalization of r electronics caused by conjugation over part or all of the molecule. When voltage is applied, electrons from the highest occupied molecular orbital (HOMO) present at the anode flow into the lowest unoccupied molecular orbital (LUMO) of the organic molecules present at the cathode. Removal of electrons from the HOMO is also referred to as inserting electron holes into the HOMO. Electrostatic forces bring the electrons and the holes towards each other until they recombine and form an exciton (which is the bound state of the electron and the hole). As the excited state decays and the energy levels of the electrons relax, radiation is emitted having a frequency in the visible spectrum. The frequency of this radiation depends on the band gap of the material, which is the difference in energy between the HOMO and the LUMO.

As electrons and holes are fermions with half integer spin, an exciton may either be in a singlet state or a triplet state depending on how the spins of the electron and hole have been combined. Statistically, three triplet excitons will be formed for each singlet exciton. Decay from triplet states is spin forbidden, which results in increases in the timescale of the transition and limits the internal efficiency of fluorescent devices. Phosphorescent organic light-emitting diodes make use of spin-orbit interactions to facilitate intersystem crossing between singlet and triplet states, thus obtaining emission from both singlet and triplet states and improving the internal efficiency.

The prototypical phosphorescent material is iridium tris(2-phenylpyridine) (Ir(ppy)₃) in which the excited state is a charge transfer from the Ir atom to the organic ligand. Such approaches have reduced the triplet lifetime to about 1 μs, several orders of magnitude slower than the radiative lifetimes of fully-allowed transitions such as fluorescence. Ir-based phosphors have proven to be acceptable for many display applications, but losses due to large triplet densities still prevent the application of OLEDs to solid-state lighting at higher brightness.

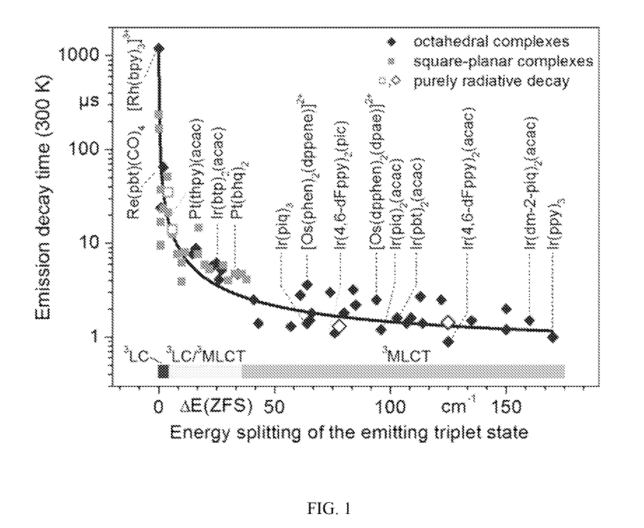

Further, recent research suggests that traditional Iridium based OLEDs may have reached a physical performance limit. As illustrated in FIG. 1, the brightness of an OLED will decrease as the time of decay increases. Since the highest energy triplet state is the origin of the luminescent transition in the Ir-based materials of FIG. 1, increasing the zero-field splitting through additional spin-orbit coupling will eventually lengthen the effective lifetime of the other two triplets. It is believed that this effect is responsible for the asymptote empirically observed at about 1 μs.

The recently developed thermally activated delayed fluorescence (TADF) seeks to minimize energetic splitting between singlet and triplet states (Δ). The reduction in exchange splitting from typical values of 0.4-0.7 eV to a gap of the order of the thermal energy (proportional to k_{B}T, where k_{B} represents the Boltzmann constant, and T represents temperature) means that thermal agitation can transfer population between singlet levels and triplet sublevels in a relevant timescale even if the coupling between states is small.

Example TADF molecules consist of donor and acceptor moieties connected directly by a covalent bond or via a conjugated linker (or “bridge”). A “donor” moiety is likely to transfer electrons from its HOMO upon excitation to the “acceptor” moiety. An “acceptor” moiety is likely to accept the electrons from the “donor” moiety into its LUMO. The donor-acceptor nature of TADF molecules results in low-lying excited states with charge-transfer character that exhibit very low A. Since thermal molecular motions can randomly vary the optical properties of donor-acceptor systems, a rigid three-dimensional arrangement of donor and acceptor moieties can be used to limit the non-radiative decay of the charge-transfer state by internal conversion during the lifetime of the excitation.

It is beneficial, therefore, to decrease energetic splitting between singlet and triplet states (Δ), and to create a system with increased reversed intersystem crossing (RISC) capable of exploiting triplet excitons. Such a system, it is believed, will result in decreased emission lifetimes. Systems with these features will be capable of emitting blue light without being subject to the rapid degradation prevalent in blue OLEDs known today.

### Compounds of the Invention

The molecules of the present invention, when excited via thermal or electronic means, can produce light in the blue or green region of the visible spectrum. The molecules comprise molecular fragments including at least one donor moiety, at least one acceptor moiety, and optionally, a bridge moiety.

Electronic properties of the example molecules of the present invention can be computed using known ab initio quantum mechanical computations. By scanning a library of small chemical compounds for specific quantum properties, molecules can be constructed which exhibit the desired spin-orbit/thermally activated delayed fluorescence (SO/TADF) properties described above.

It could be beneficial, for example, to build molecules of the present invention using molecular fragments with a calculated triplet state above 2.75 eV. Therefore, using a time-dependent density functional theory using, as a basis set, the set of functions known as 6-31 G* and a Becke, 3-parameter, Lee-Yang-Parr hybrid functional to solve Hartree-Fock equations (TD-DFT/B3LYP/6-31G*), molecular fragments (moieties) can be screened which have HOMOs above a specific threshold and LUMOs below a specific threshold, and wherein the calculated triplet state of the moieties is above 2.75 eV.

Therefore, for example, a donor moiety (“D”) can be selected because it has a HOMO energy (e.g., an ionization potential) of greater than or equal to −6.5 eV. An acceptor moiety (“A”) can be selected because it has, for example, a LUMO energy (e.g., an electron affinity) of less than or equal to −0.5 eV. The bridge moiety (“B”) can be a rigid conjugated system which can, for example, sterically restrict the acceptor and donor moieties into a specific configuration, thereby preventing the overlap between the conjugated x system of donor and acceptor moieties.

Accordingly, in a first aspect, the present invention is a molecule comprising at least one acceptor moiety A, at least one donor moiety D, and optionally, a bridge moiety B. The moiety D, for each occurrence independently, is a monocyclic or fused polycyclic aryl or heteroaryl having between 5 and 20 atoms, optionally substituted with one or more substituents. The moiety A, for each occurrence independently, is —CF₃, —CN, or a monocyclic or fused polycyclic aryl or heteroaryl having between 5 and 20 atoms, optionally substituted with one or more substituents. The moiety B, for each occurrence independently, is phenyl optionally substituted with one to four substituents. Each moiety A is covalently attached to either the moiety B or the moiety D, each moiety D is covalently attached to either the moiety B or the moiety A, and each moiety B is covalently attached to at least one moiety A and at least one moiety D. In an example embodiment of the first aspect, each moiety A is bonded either to moiety B or moiety D, each moiety B is bonded either to moiety A, moiety D, or a second moiety B, and each moiety D is bonded either to moiety A or moiety B. In another example embodiment of the first aspect, the moieties A are different than the moieties D.

The foregoing rules of connection mean that the moiety A cannot be connected to another moiety A, the moiety D cannot be connected to another moiety D, and that each moiety B is multivalent, and therefore must be connected to at least two other moieties, either a moiety A, a moiety D, or a second moiety B. It is understood that within a molecule no molecular fragment represented by A is the same as any molecular fragment represented by D.

In a second aspect, the present invention is a molecule comprising at least one acceptor moiety A, at least one donor moiety D, and optionally, one or more bridge moieties B, wherein A, D, and B are defined above with respect to the first aspect of the present invention. In addition to the moieties recited above in the first aspect, the moiety D can be —N(C₆-C₁₈aryl)₂. In addition to the moieties recited above with respect to the first aspect, the moiety A, can be —S(O)₂—. In addition to the moieties recited above with respect to the first aspect, the moiety B can be C₂-C₆ alkenyl, C₂-C₆ alkynyl, or C₅-C₁₂ cycloalkyl optionally substituted with one to four substituents.

In a third aspect, the present invention is a molecule defined by the structural formula (I)

(A)ₘ-(B)ₗ-(D)ₚ (I)

wherein A, B, and D are defined above with respect to the first and second aspects, and

the moiety D, for each occurrence independently, is optionally substituted with one or more substituents each independently selected from C₁-C₆ alkyl, C₂-C₆ alkenyl, C₂-C₆ alkynyl, C₆-C₁₈ aryl, (5-20 atom) heteroaryl, C₁-C₆ alkoxy, amino, C₁-C₃ alkylamino, C₁-C₃ dialkylamino, or oxo;

the moiety A, for each occurrence independently, is optionally substituted with one or more substituents independently selected from C₁-C₆ alkyl, C₂-C₆ alkenyl, C₂-C₆ alkynyl, C₆-C₁₈ aryl, (5-20 atom) heteroaryl, C₁-C₆ alkoxy, —C(O)C₁-C₃ haloalkyl, —S(O₂)H, —NO₂, —CN, oxo, halogen, or C₆-C₁₈ haloaryl;

the moiety B, for each occurrence independently, is optionally substituted with one to four substituents, each independently selected from C₁-C₆ alkyl, C₂-C₆ alkenyl, C₂-C₆ alkynyl, C₆-C₁₈ aryl, or (5-20 atom) heteroaryl;

m is an integer greater than 1;

p is an integer greater than 1; and

l is either 0 or an integer greater than one. In an example embodiment, l is greater than 1. In another example embodiment, l is 0, 1, or 2.

In a fourth aspect, the present invention is a molecule defined by the structural formula (I)

(A)ₘ-(B)ₗ-(D)ₚ (I)

wherein A, B, and D are defined above with respect to the first or second aspects of the present invention, and

the moiety D, for each occurrence independently, is optionally substituted, in addition to the substituents described above with respect to the third aspect of the present invention, with —N(C₆-C₁₈ aryl)₂;

the moiety A, for each occurrence independently, is optionally substituted as described above with respect to the third aspect of the present invention;

the moiety B, for each occurrence independently, is optionally substituted as described above with respect to the third aspect of the present invention;

m is an integer greater than 1;

p is an integer greater than 1; and

l is either 0 or an integer greater than one. In an example embodiment, l is greater than 1. In another example embodiment, l is 0, 1, or 2.

In a fifth aspect, the present invention is molecule defined by the structural formula (I)

(A)ₘ-(B)ₗ-(D)ₚ (I)

wherein A, B, and D are defined above with respect to the first and second aspects of the present invention, and

the moiety D, for each occurrence independently, is optionally substituted as described above with respect to the third and fourth aspects, and further wherein, each alkyl, alkenyl, alkynyl, aryl, and heteroaryl optionally further substituted with one or more substituents selected from C₁-C₆ alkyl, 5-20 atom heteroaryl, or —N(C₆-C₁₈aryl)₂;

the moiety A, for each occurrence independently, is optionally substituted as described above with respect to the third aspect of the present invention;

the moiety B, for each occurrence independently, is optionally substituted as described above with respect to the third aspect of the present invention;

m is an integer greater than 1;

p is an integer greater than 1; and

l is either 0 or an integer greater than one. In an example embodiment, l is greater than 1. In another example embodiment, l is 0, 1, or 2.

In a sixth aspect, the present invention is a molecule as defined above with respect to the first or second aspects of the present invention, and wherein the moiety D, for each occurrence independently, can be selected from List D1.

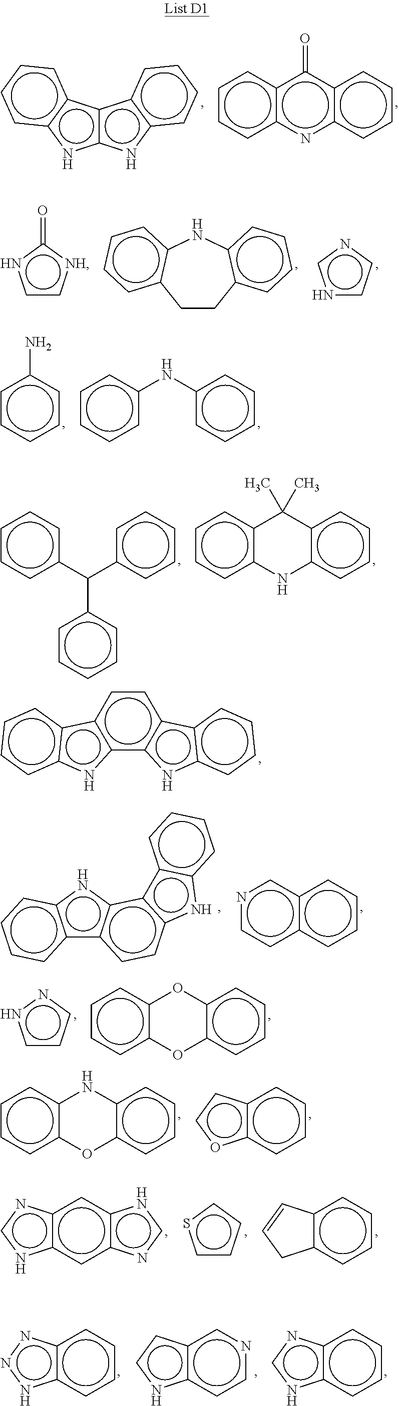

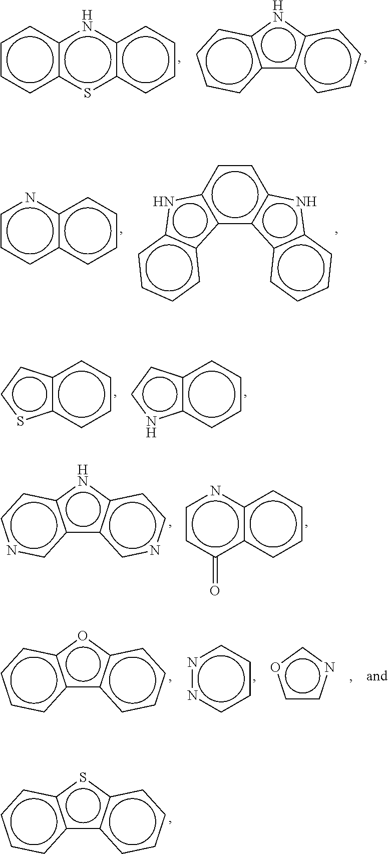

and wherein the moiety D can be optionally substituted as described above with respect to the third, fourth, and fifth aspects of the present invention.

In a seventh aspect, the present invention is a molecule as defined above with respect to the first or second aspects of the present invention, and wherein the moiety D, for each occurrence independently, can be selected from List D1, List D2, or both.

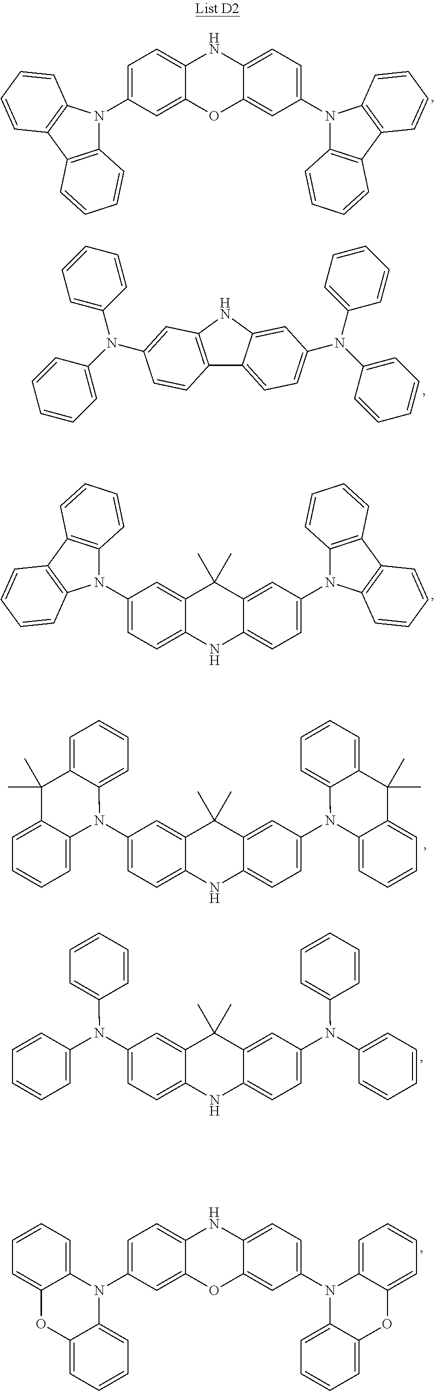

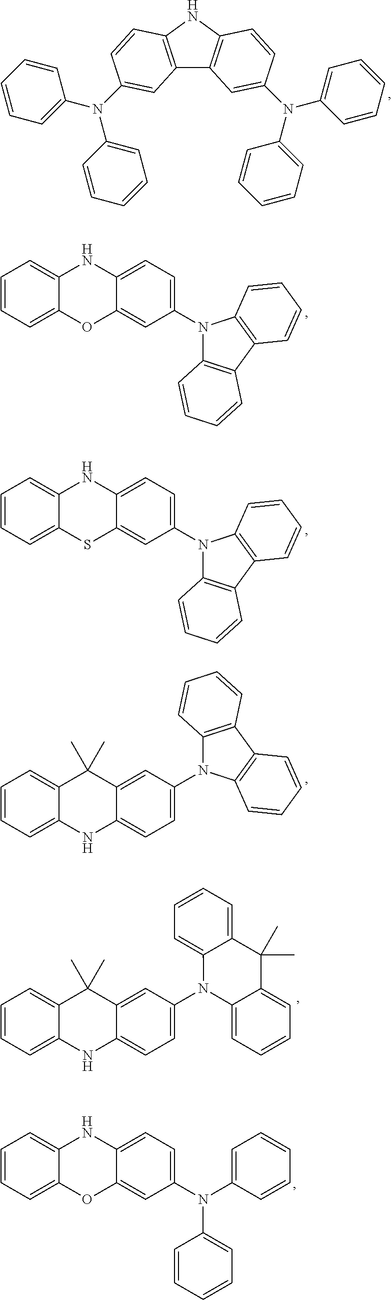

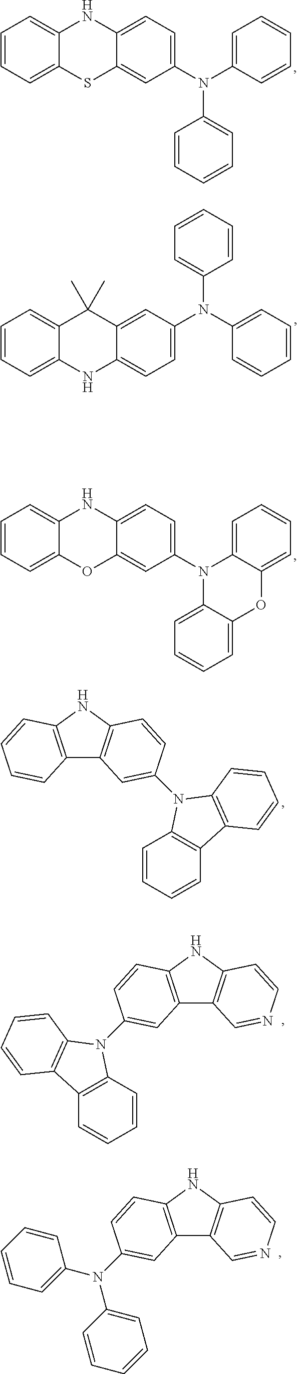

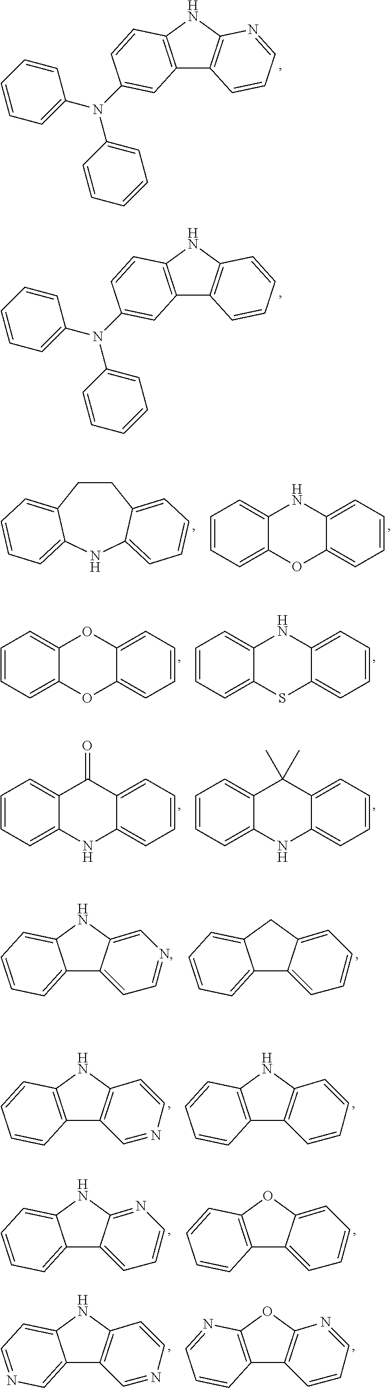

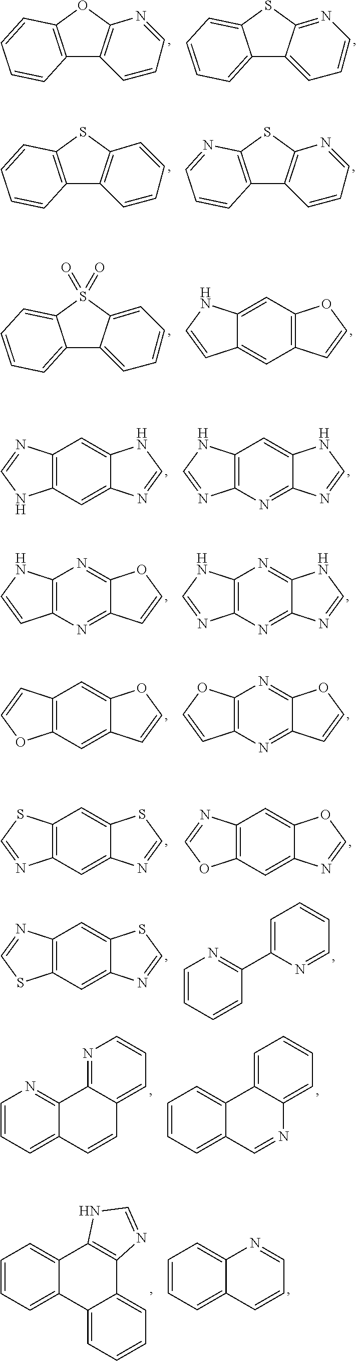

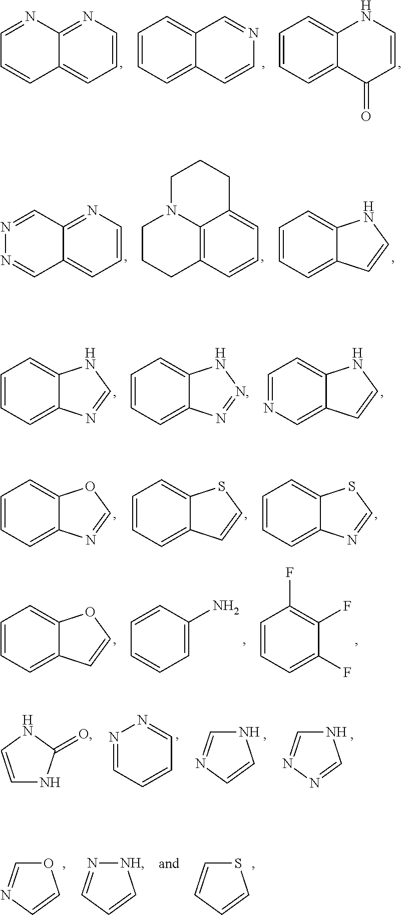

and wherein the moiety D can be optionally substituted as described above with respect to the third, fourth, and fifth aspects of the present invention.

In a eighth aspect, the present invention is a molecule as defined above with respect to the first or second aspects of the present invention, and wherein the moiety D, for each occurrence independently, can be selected from List D1, List D2, List D3, or any combination thereof.

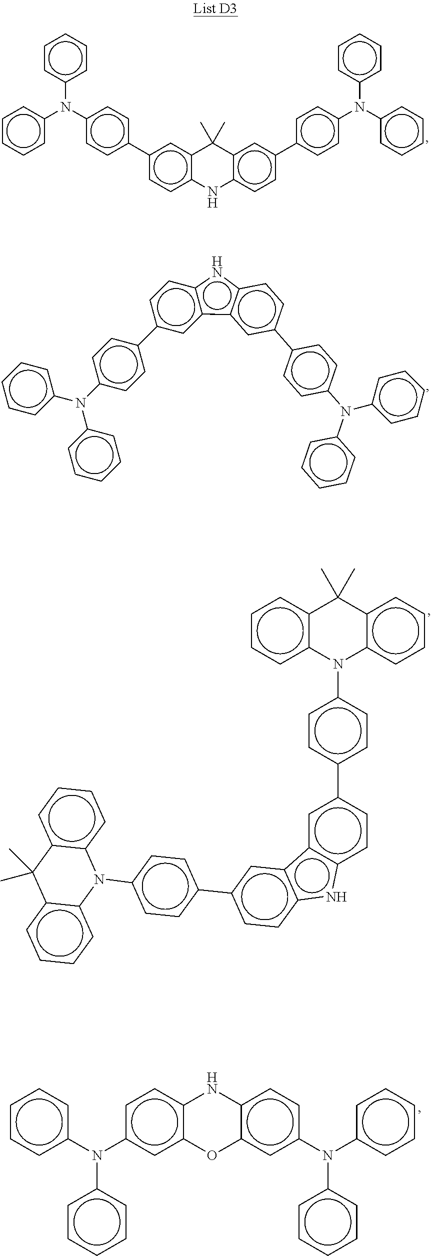

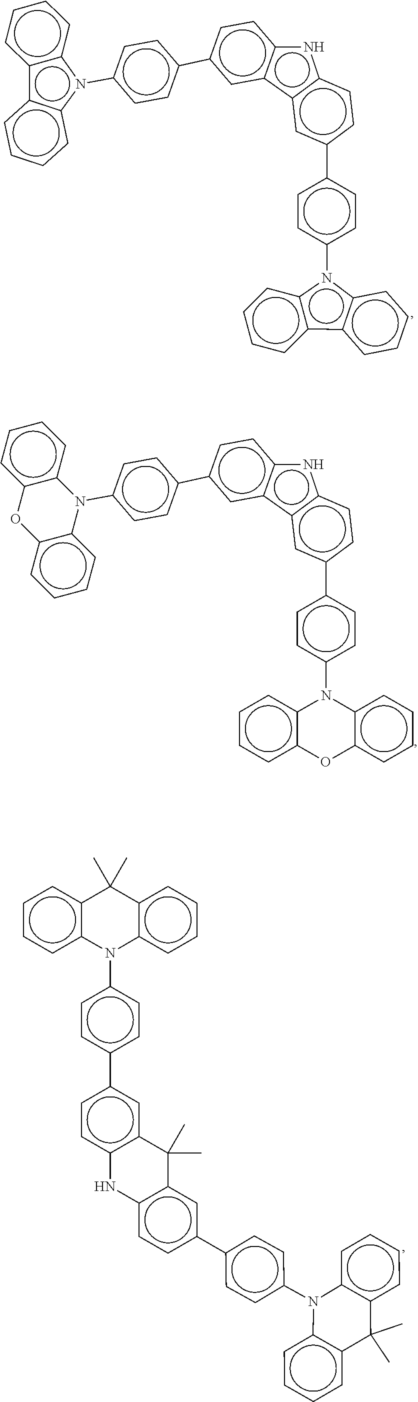

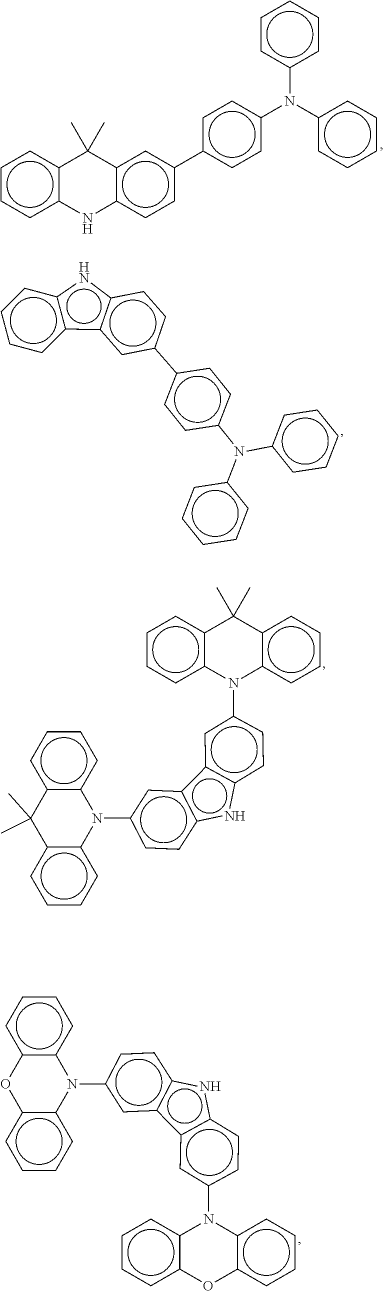

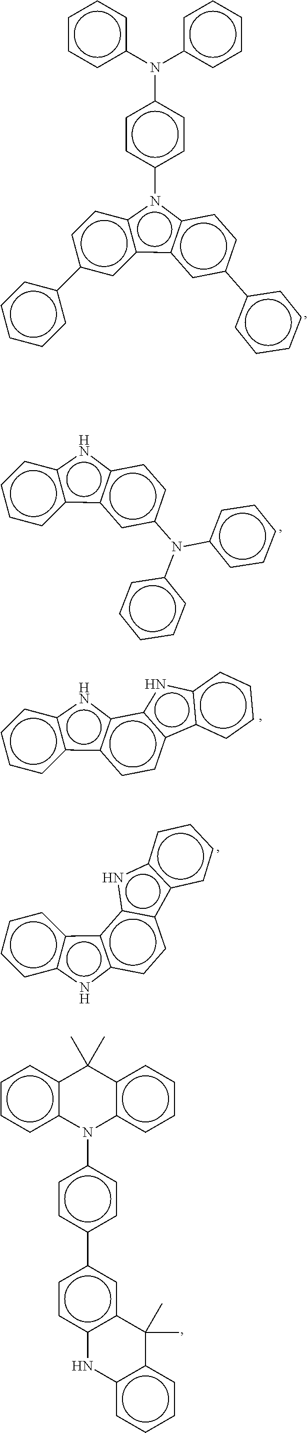

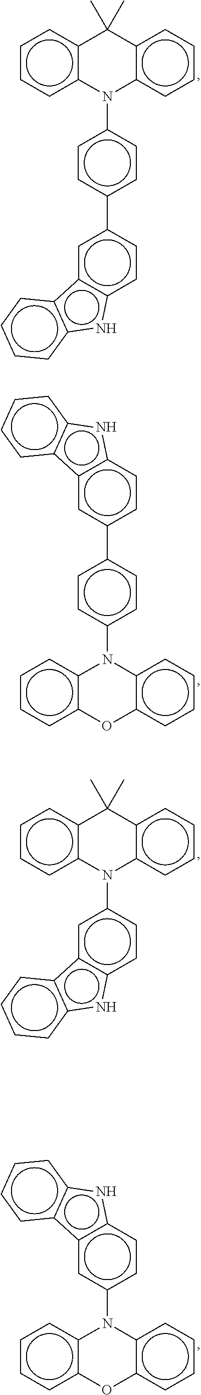

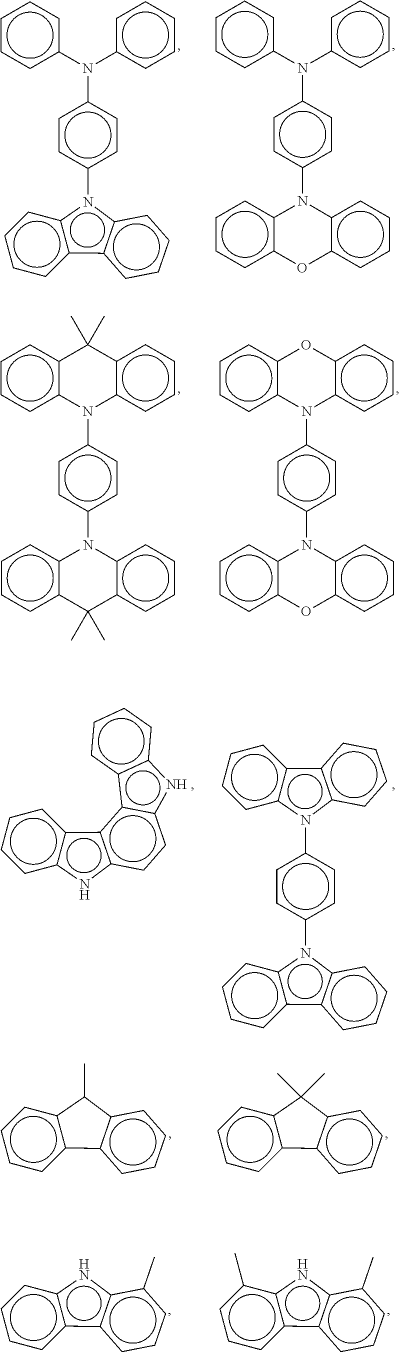

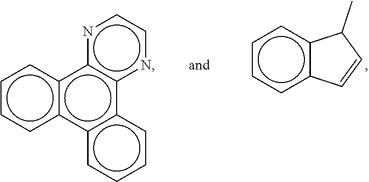

and wherein the moiety D can be optionally substituted as described above with respect to the third, fourth, and fifth aspects of the present invention.

In an ninth aspect, the present invention is a molecule as defined above with respect to the first or second aspects of the present invention, and wherein the moiety A, for each occurrence independently, can be selected from List A1.

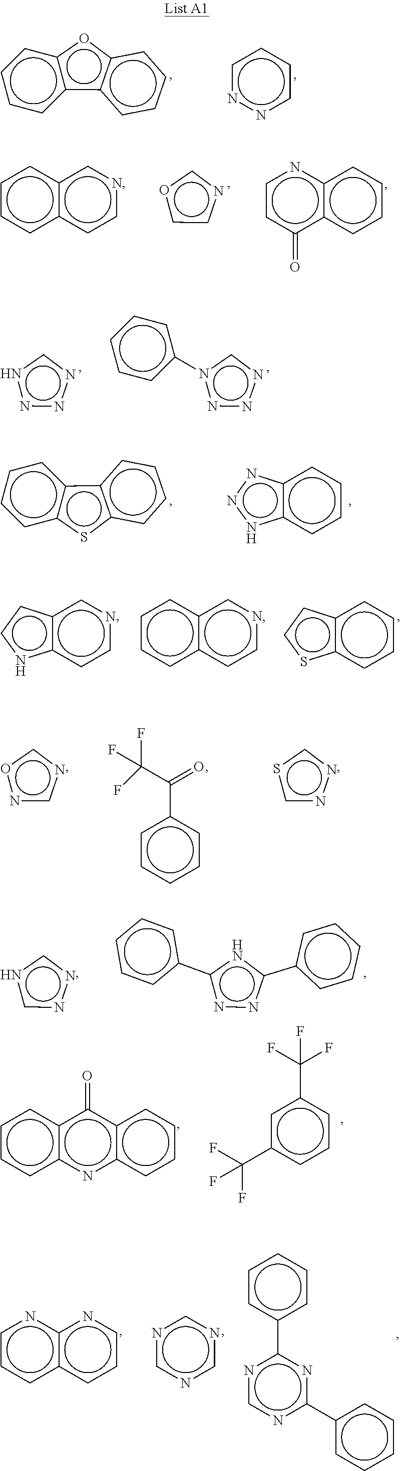

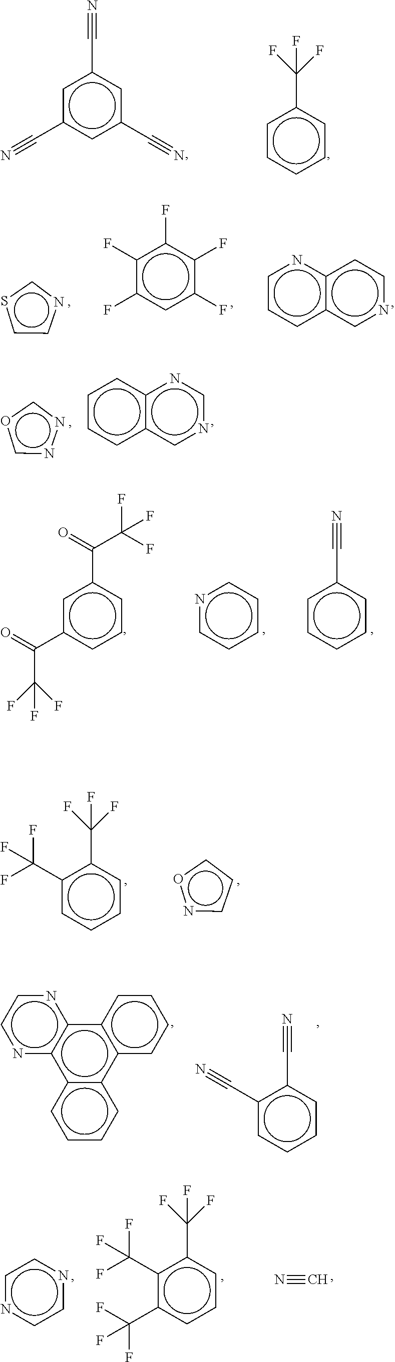

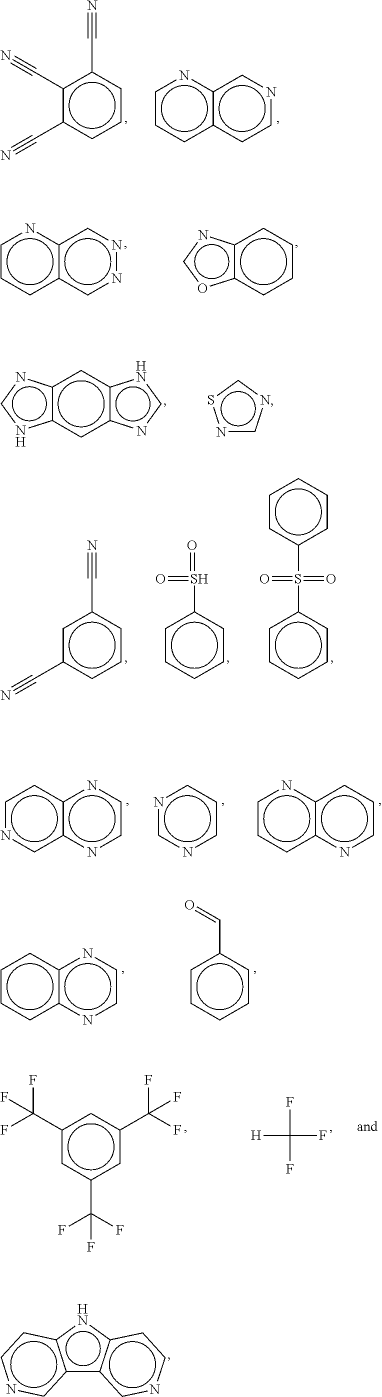

and wherein the moiety A can be optionally substituted as described above with respect to the third, fourth, and fifth aspects of the present invention.

In a tenth aspect, the present invention is a molecule as defined above with respect to the first, second, third, aspects of the present invention, and wherein the moiety A, for each occurrence independently, can be selected from List A1, List A2, or both.

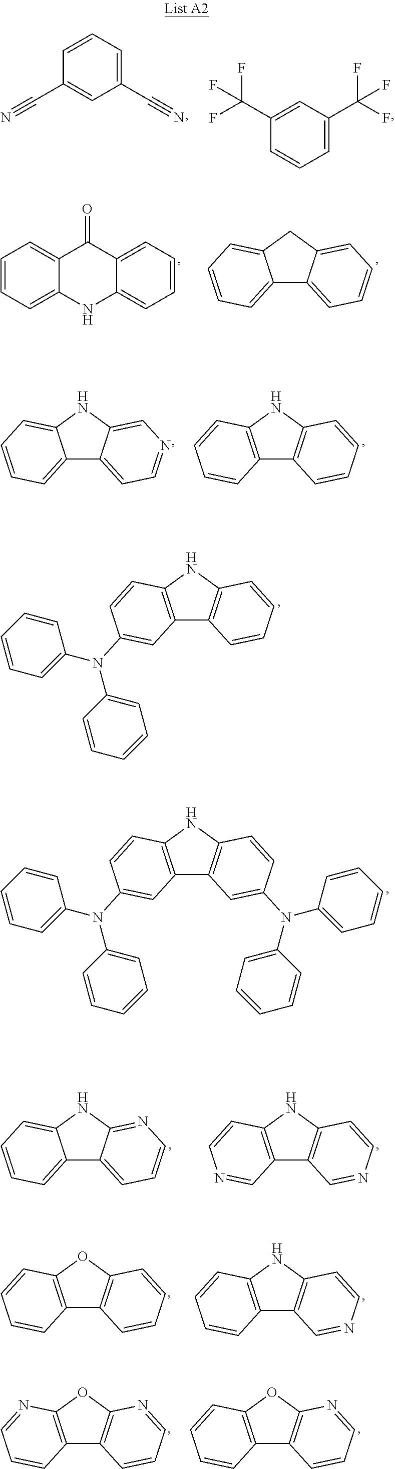

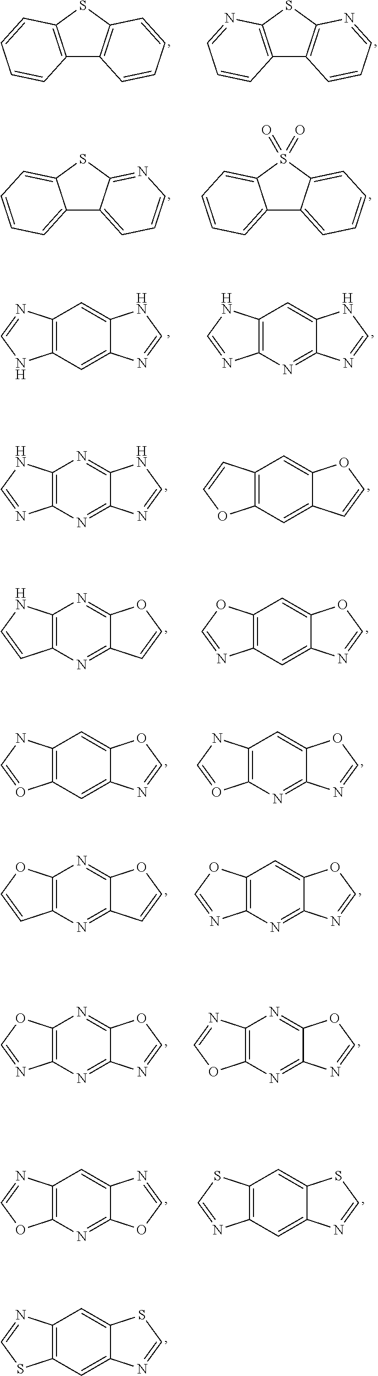

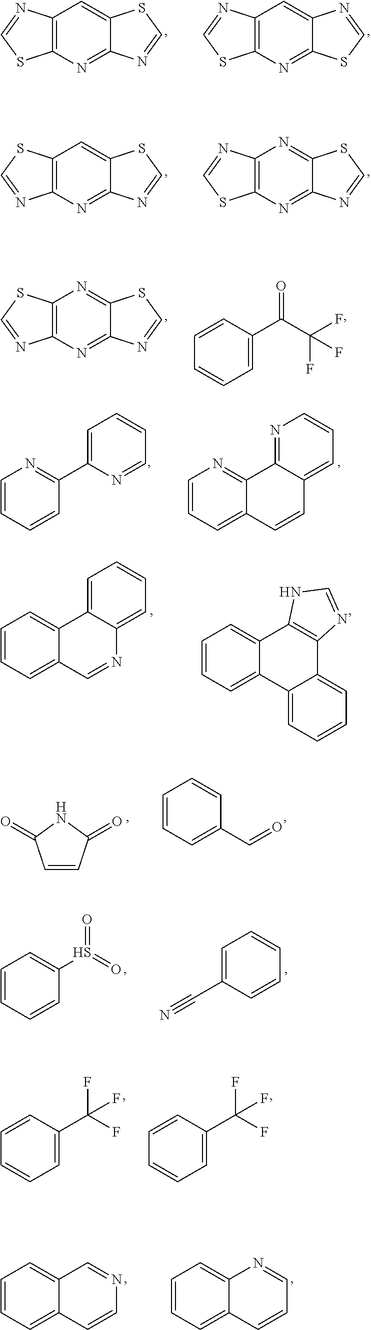

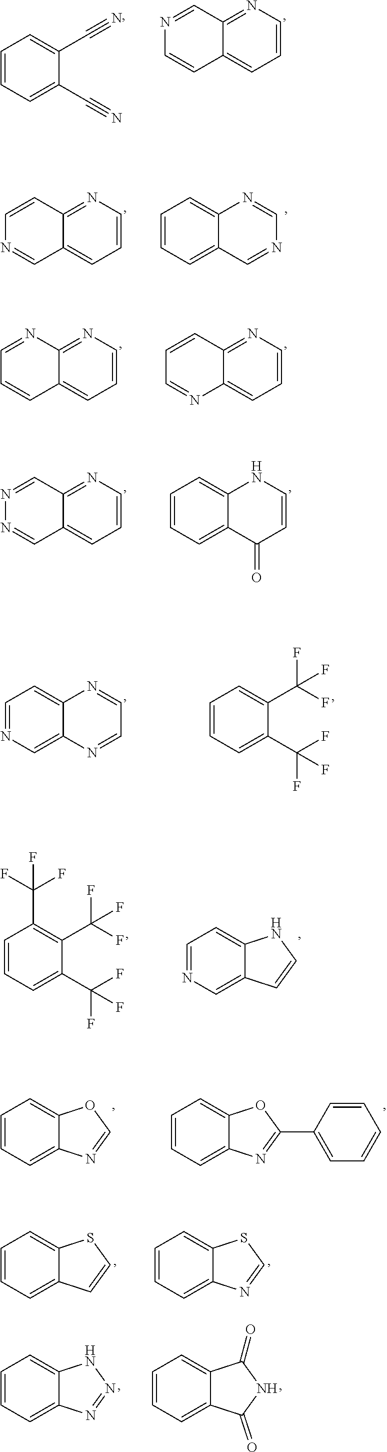

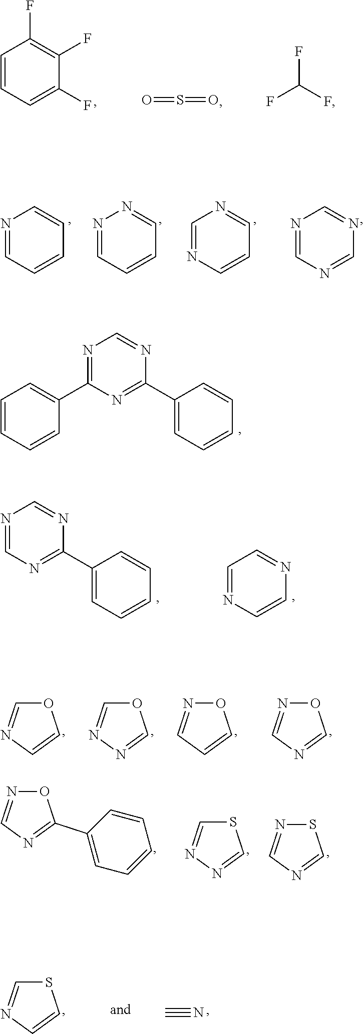

and wherein the moiety A can be optionally substituted as described above with respect to the third, fourth, and fifth aspects of the present invention.

In a eleventh aspect, the present invention is a molecule as defined above with respect to the first or second aspects of the present invention, and wherein the moiety A, for each occurrence independently, can be selected from List A1, List A2, List A3, or any combination thereof.

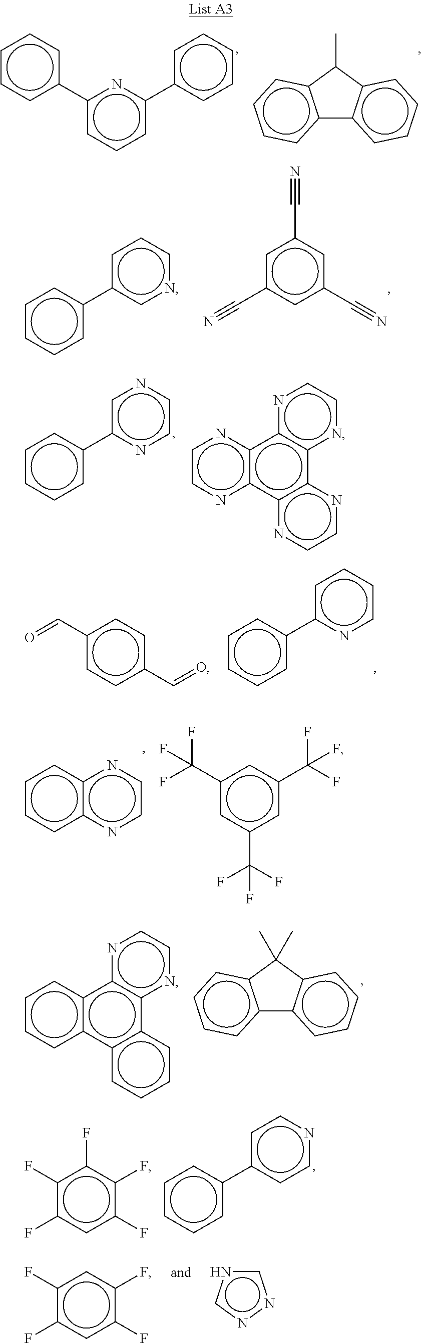

and wherein the moiety A can be optionally substituted as described above with respect to the third, fourth, and fifth aspects of the present invention.

In a twelfth aspect, the present invention is a molecule as defined above with respect to the first or second aspects of the present invention, and wherein the moiety B, for each occurrence independently, can be selected from List B1:

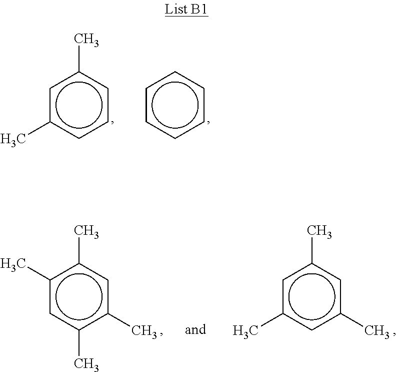

and wherein the moiety B can be optionally substituted as described above with respect to the third, fourth, and fifth aspects of the present invention.

In a thirteenth aspect, the present invention is a molecule as defined above with respect to the first or second aspects of the present invention, and wherein the moiety B, for each occurrence independently, can be selected from List B1, List B2, or both.

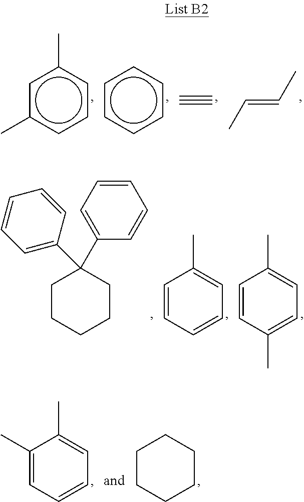

and wherein the moiety B can be optionally substituted as described above with respect to the third, fourth, and fifth aspects of the present invention.

In an example embodiment of the sixth aspect of the present invention, the moiety D, for each occurrence independently, is selected from List D4.

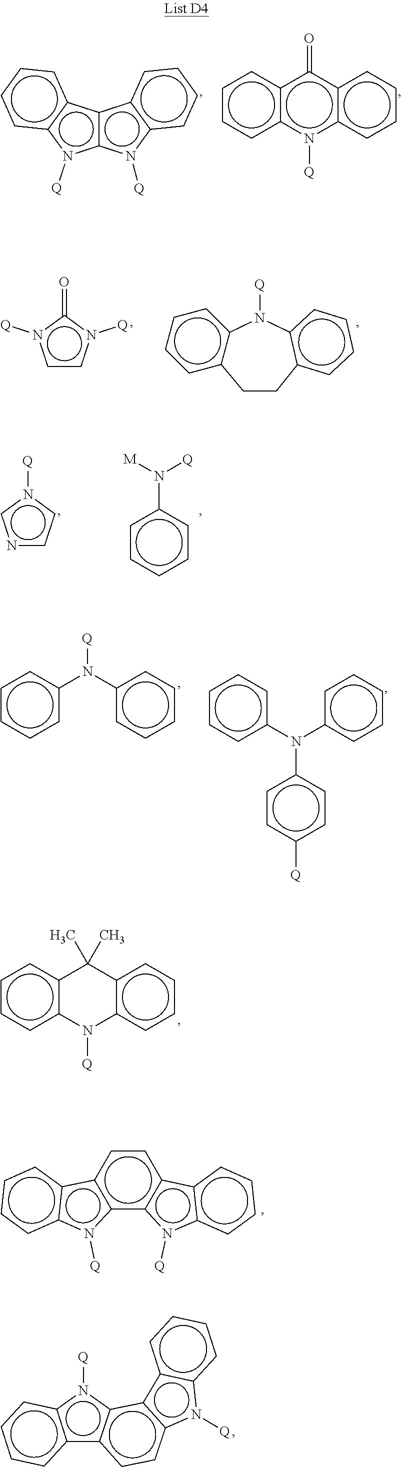

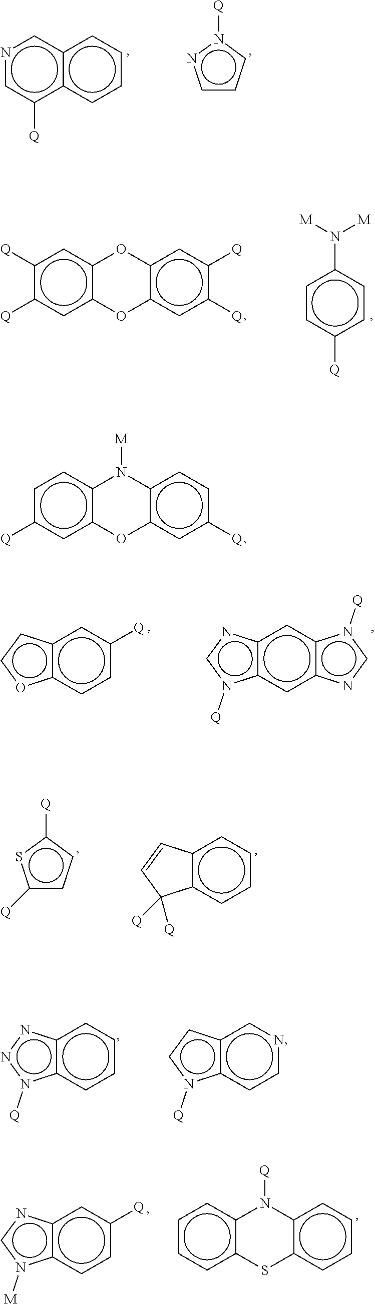

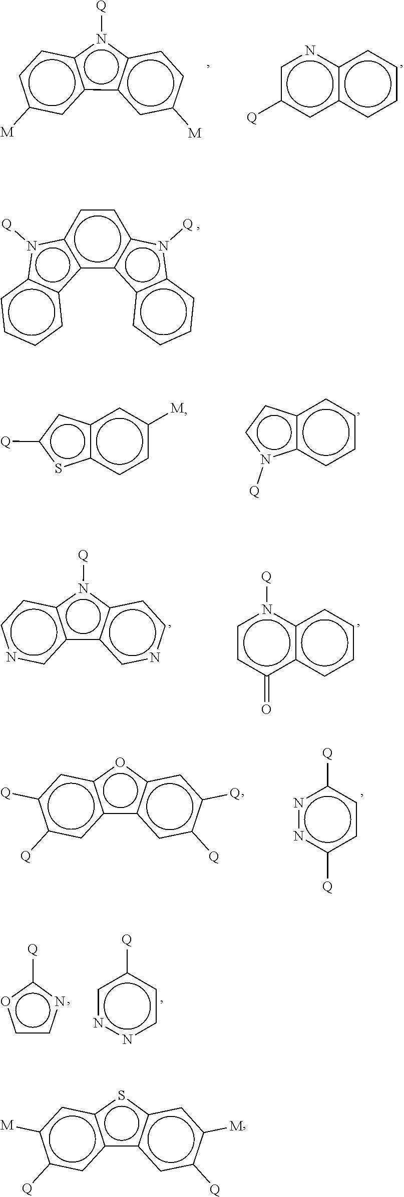

wherein, within each molecule:

Q is the moiety A or a moiety B₀₋₂-A and each M is the moiety A or the moiety B₀₋₂-A,

all groups Q are the same and all groups M are the same, and

each group Q is the same or different from any group M, and the moieties A and B are defined above with respect to the first, second, and third aspects of the present invention.

In an example embodiment of the seventh aspect of the present invention, the moiety D, for each occurrence independently, is selected from List D4, List D5, or both.

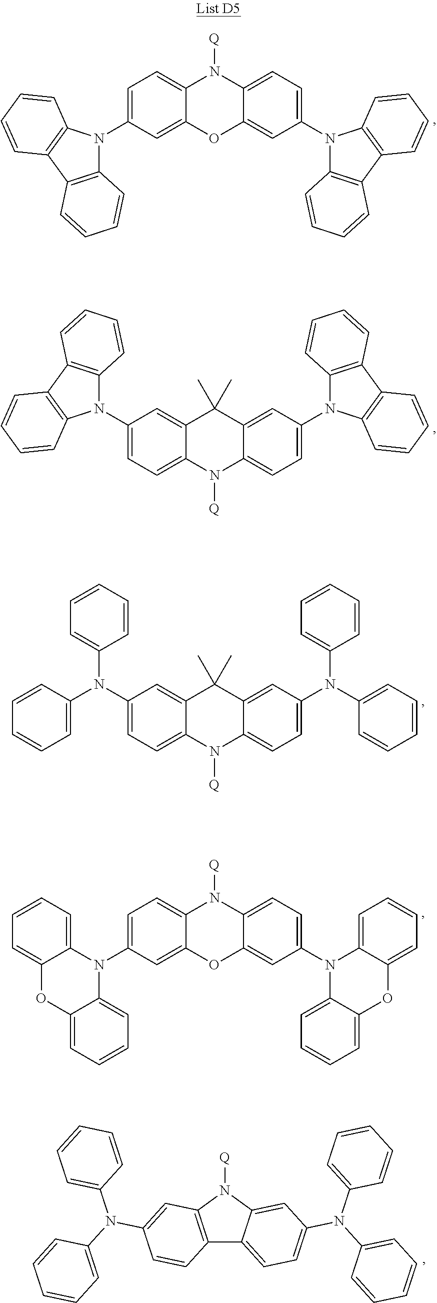

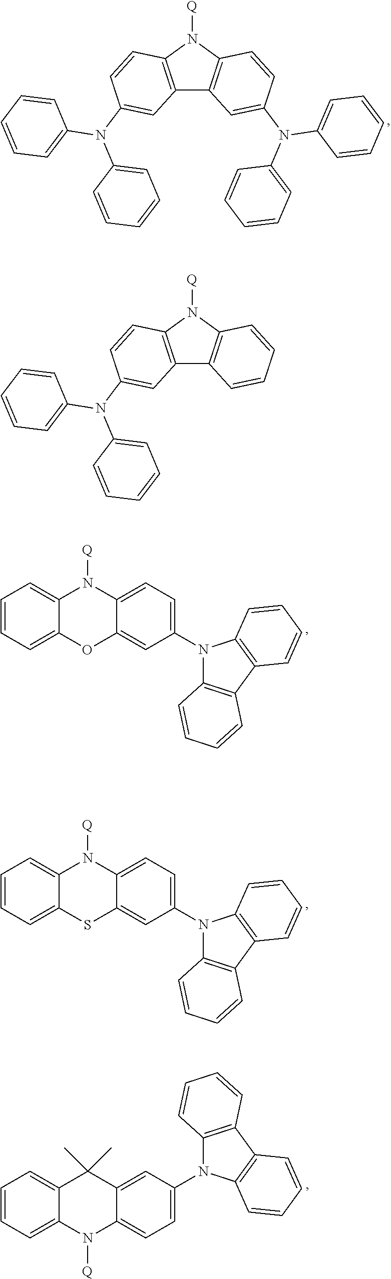

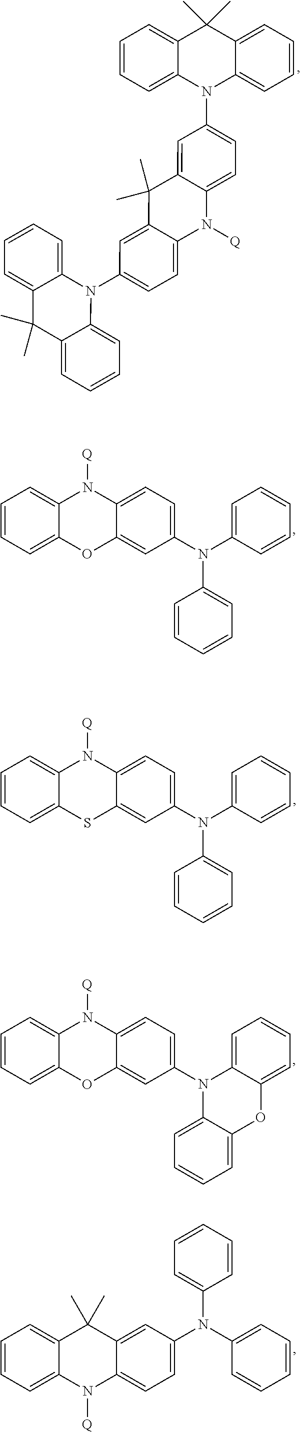

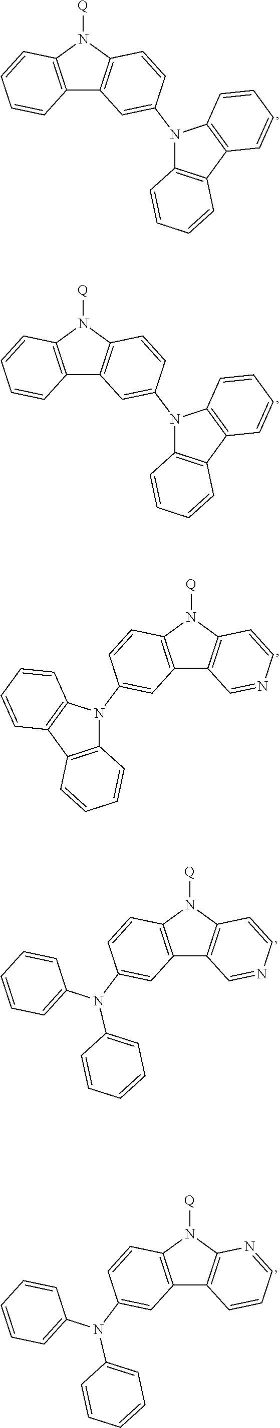

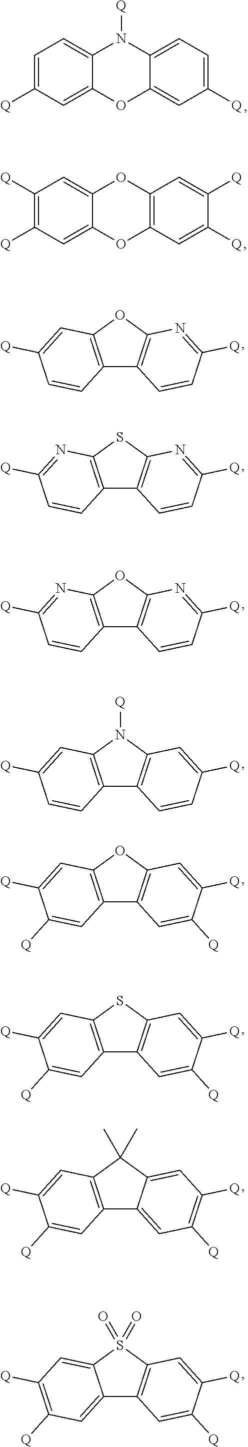

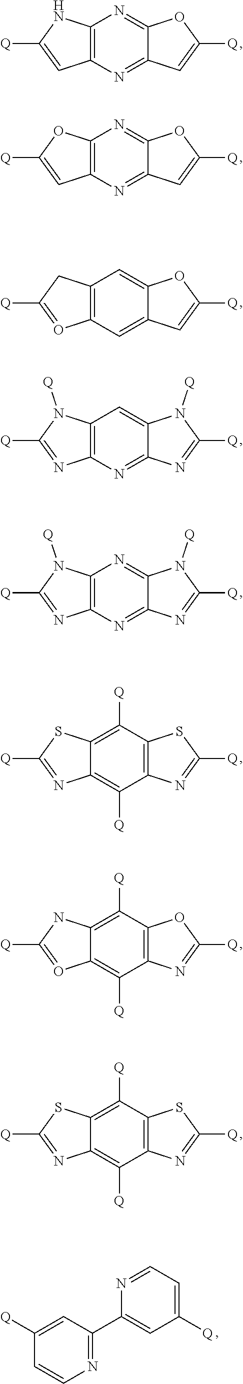

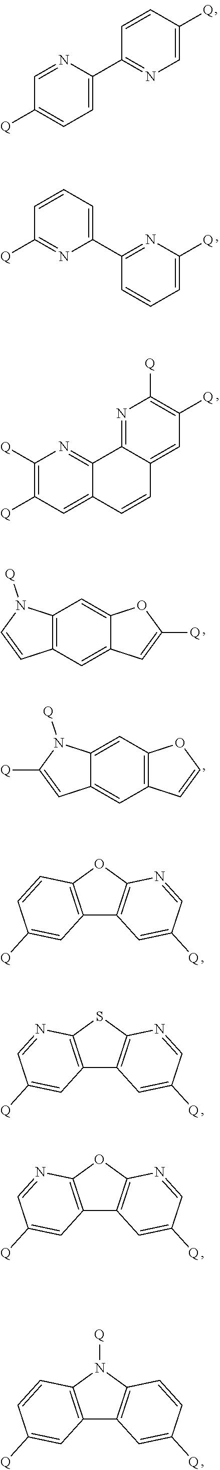

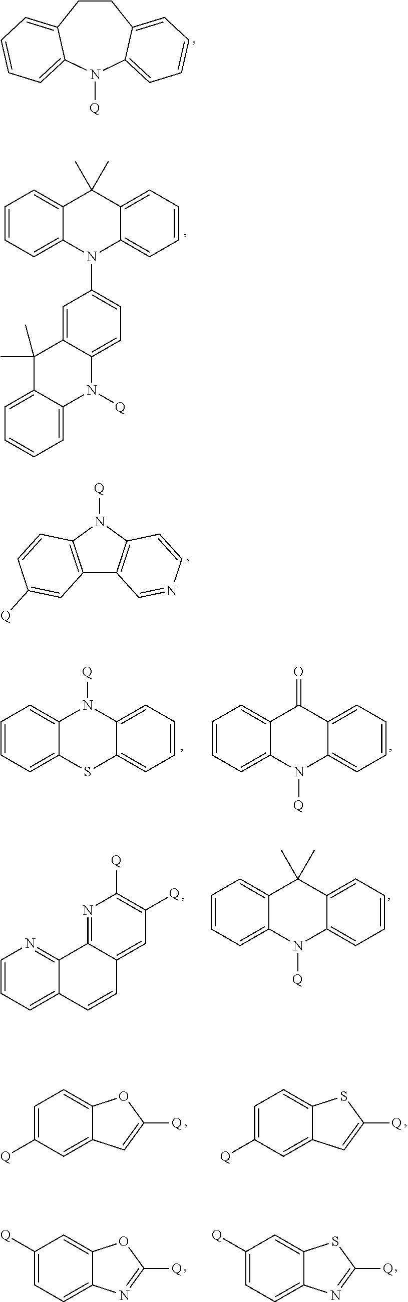

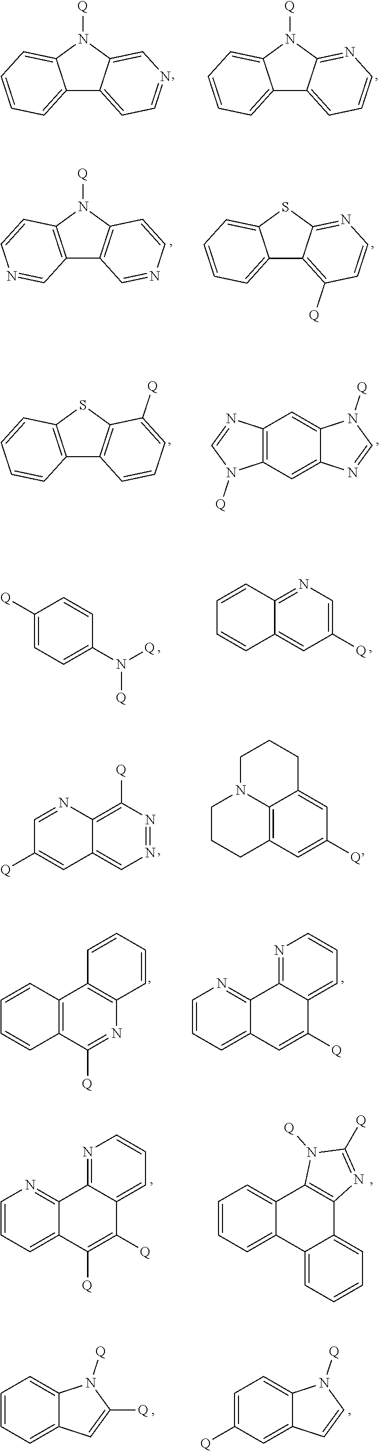

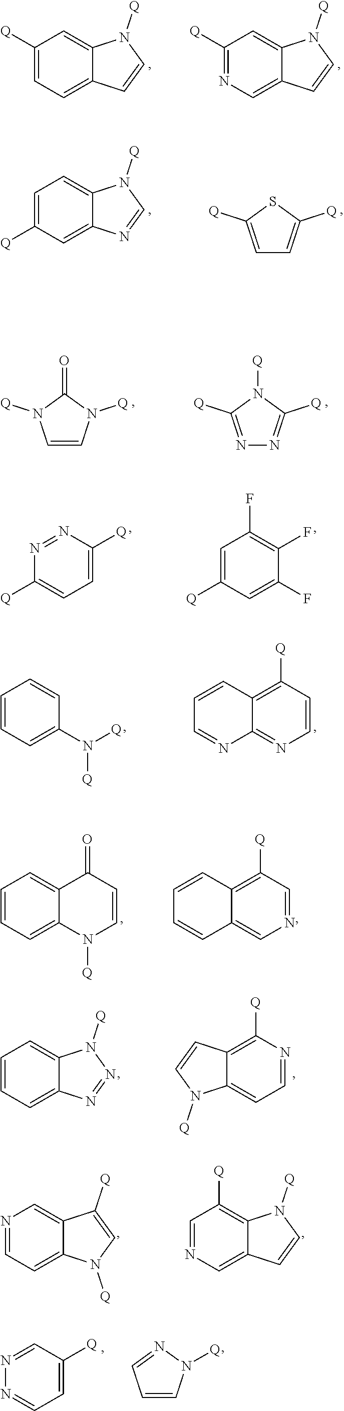

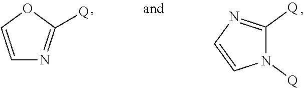

wherein, within each molecule:

Q is independently selected from the group consisting of the moiety A, a moiety B₀₋₂-A, H, C₁-C₃ alkyl, C₆-C₁₈ aryl, oxo, (5-20 atom) heteroaryl, and —N(C₆-C₁₈ aryl)₂, and wherein the moieties A and B are defined above with respect to the first, second, and third aspects of the present invention.

In an example embodiment of the seventh and eighth aspects of the present invention, the moiety D, for each occurrence independently, can also be selected from List D6.

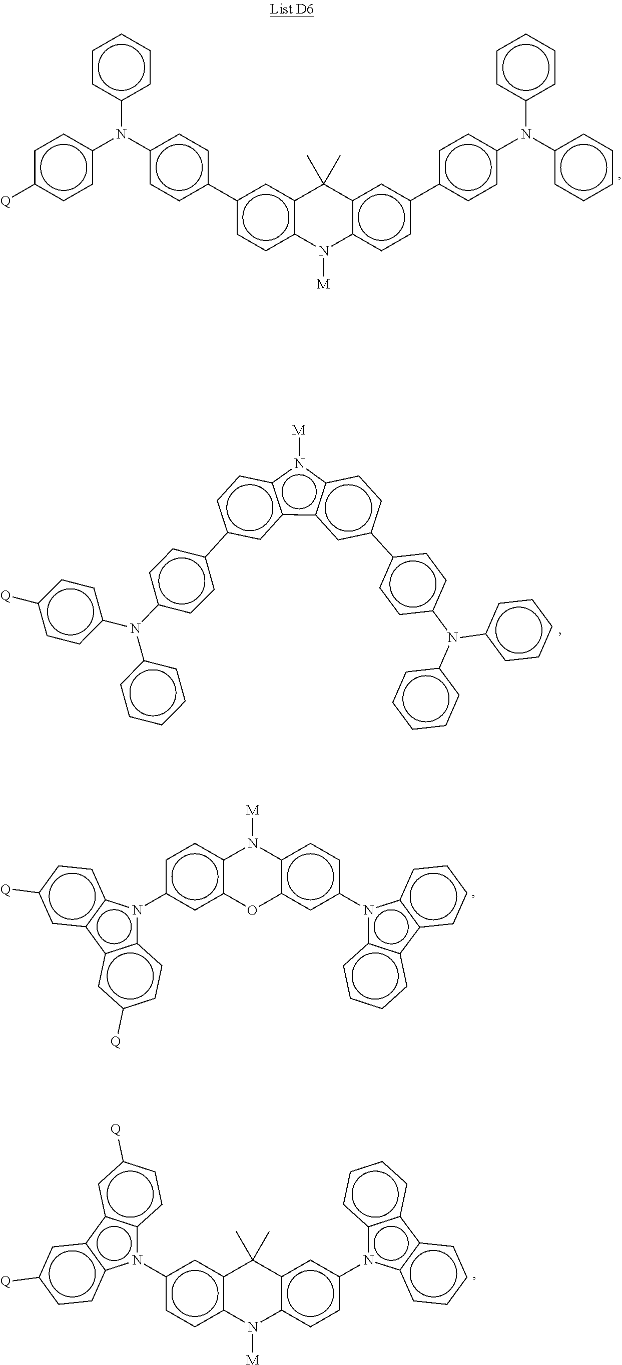

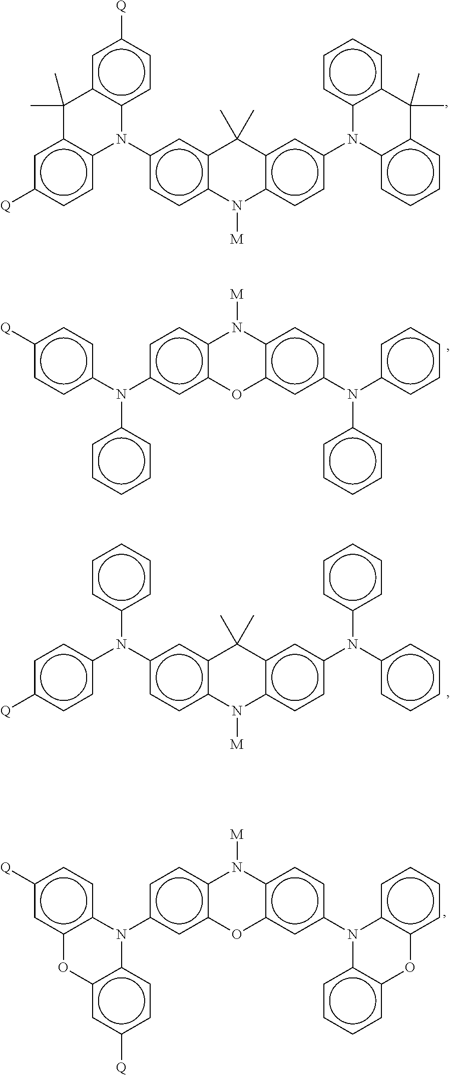

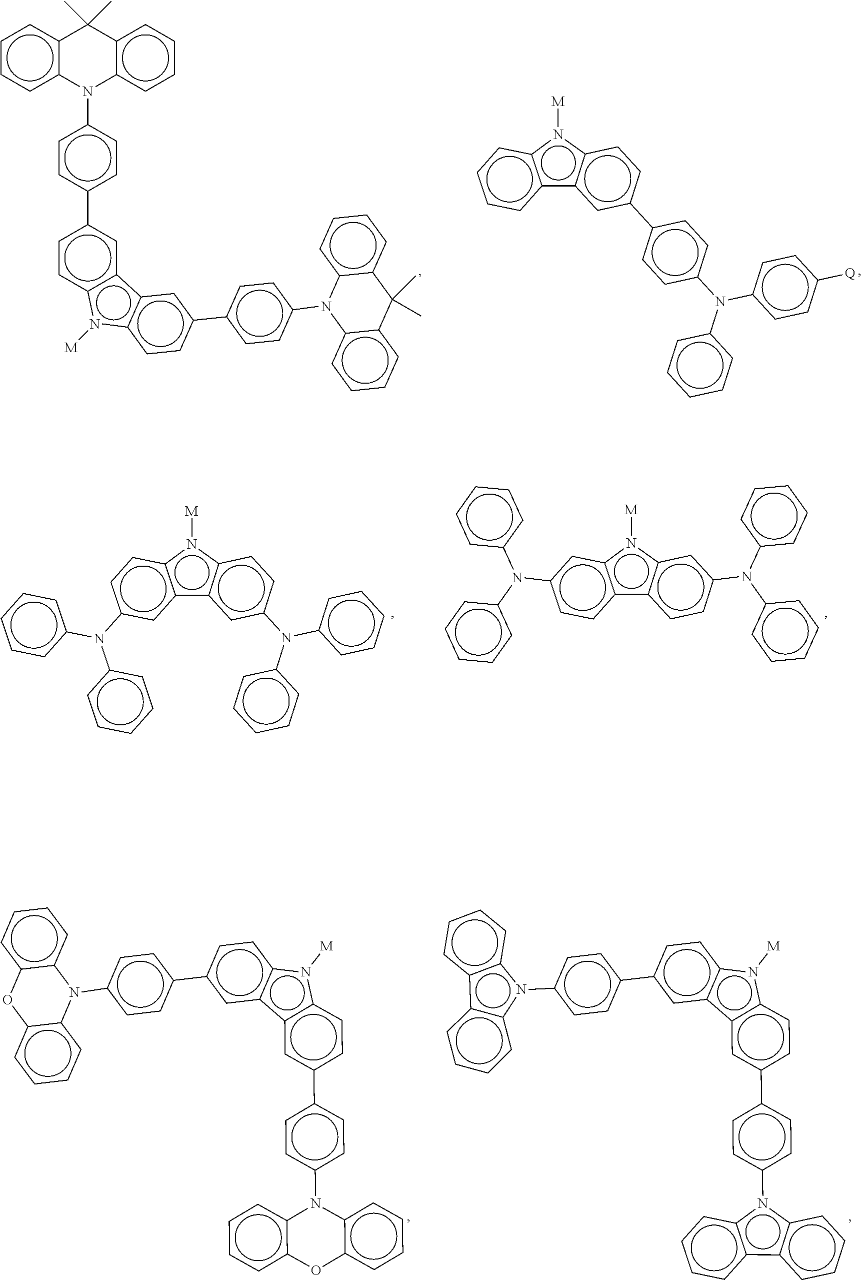

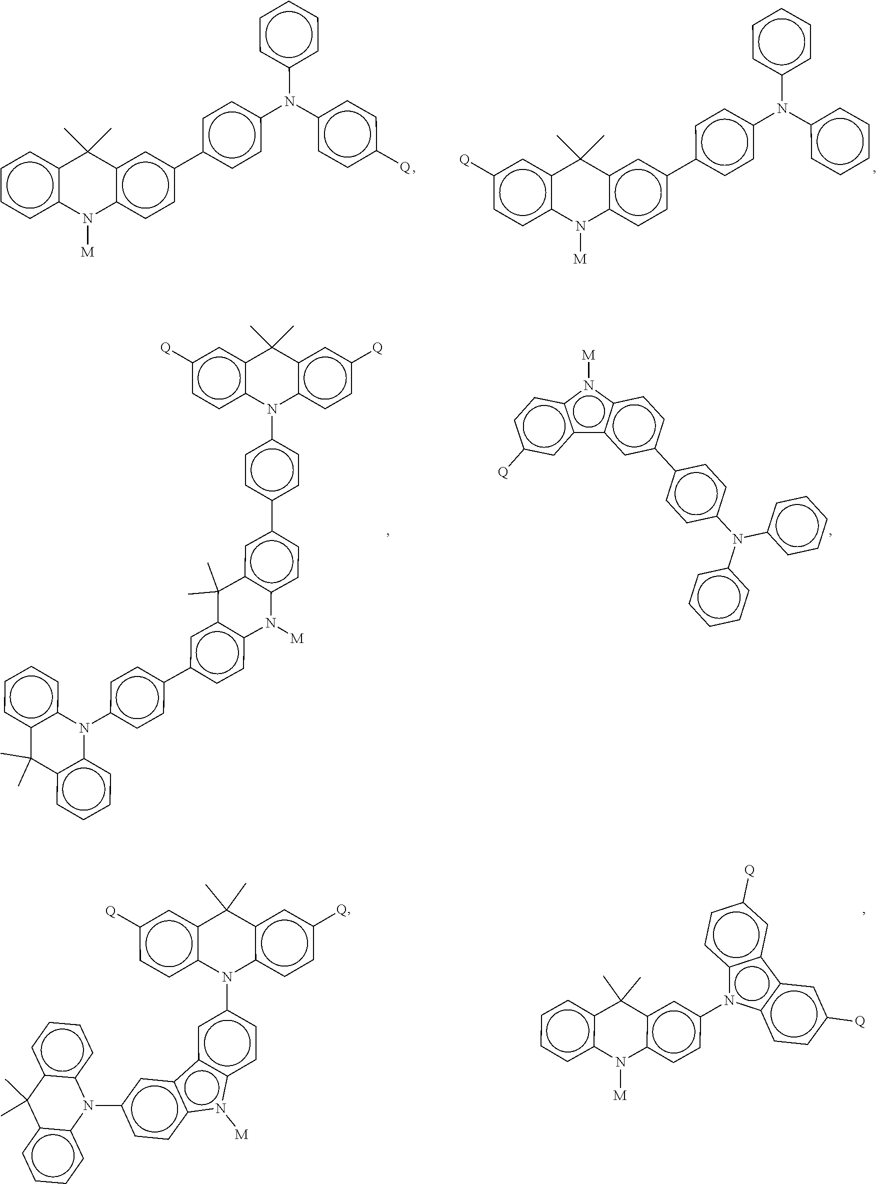

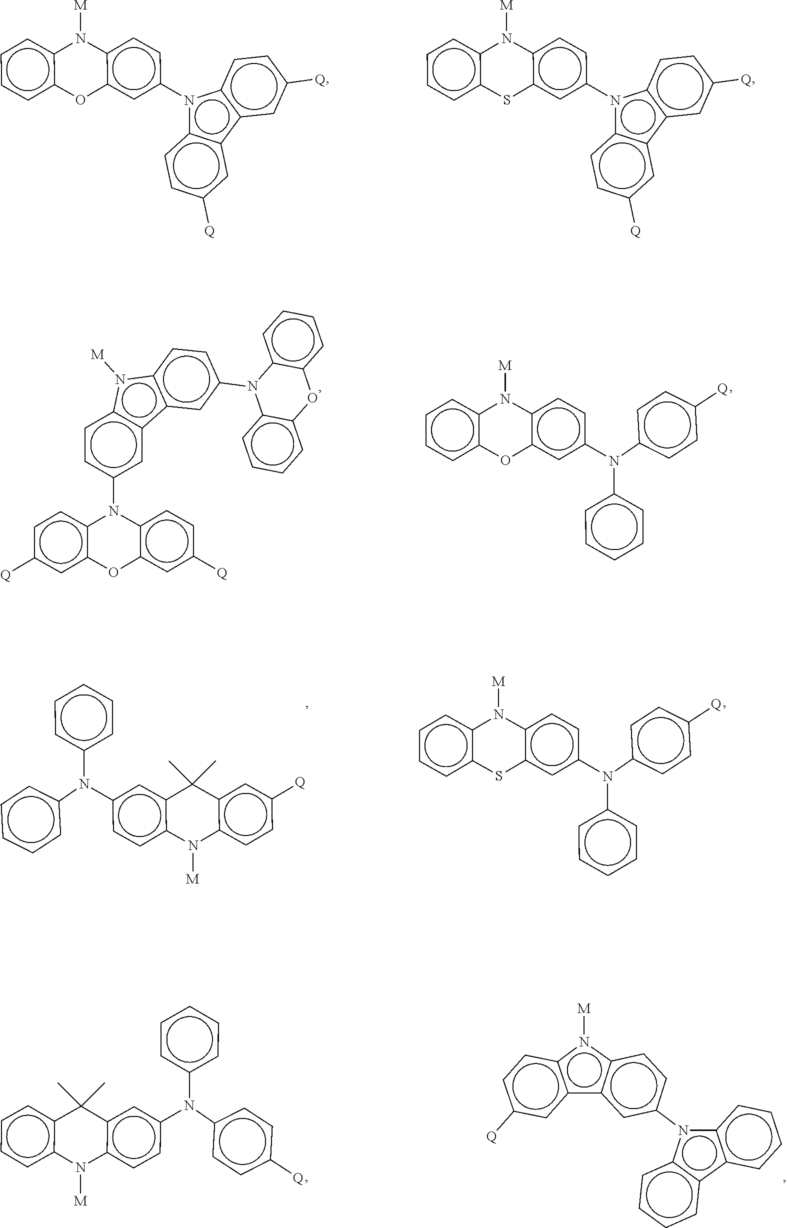

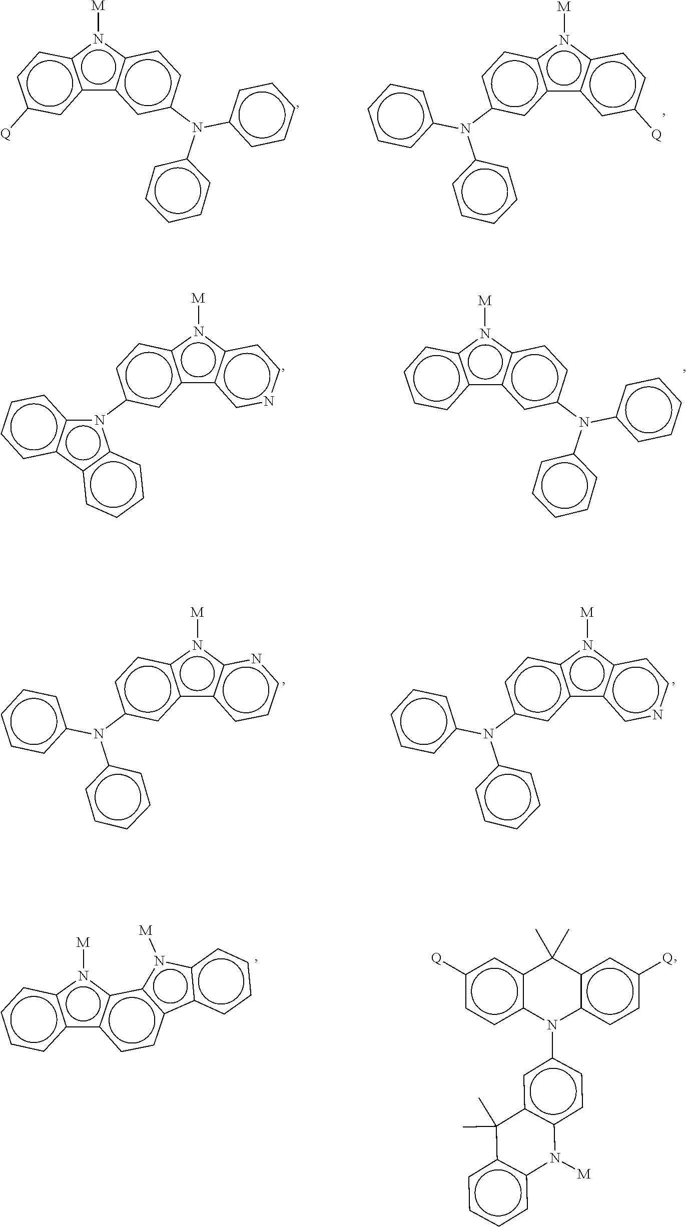

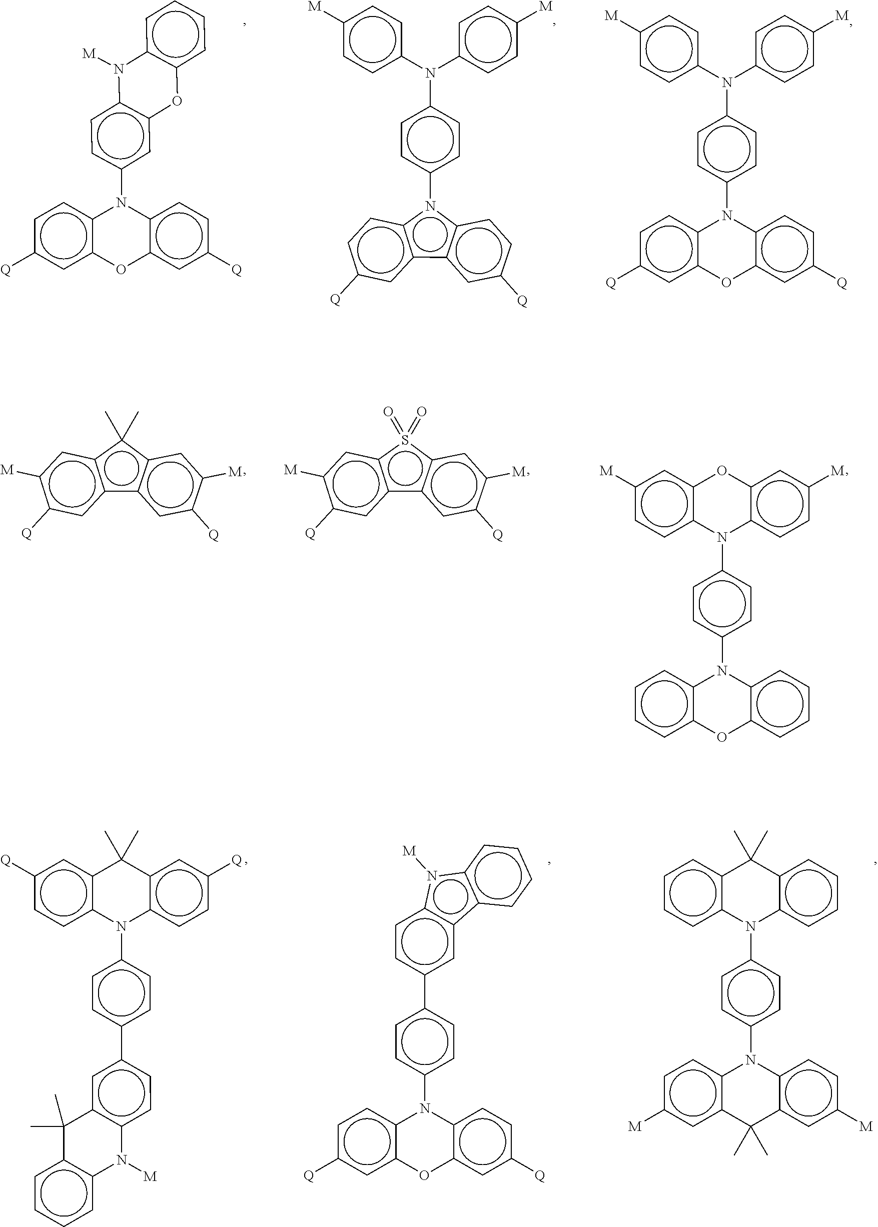

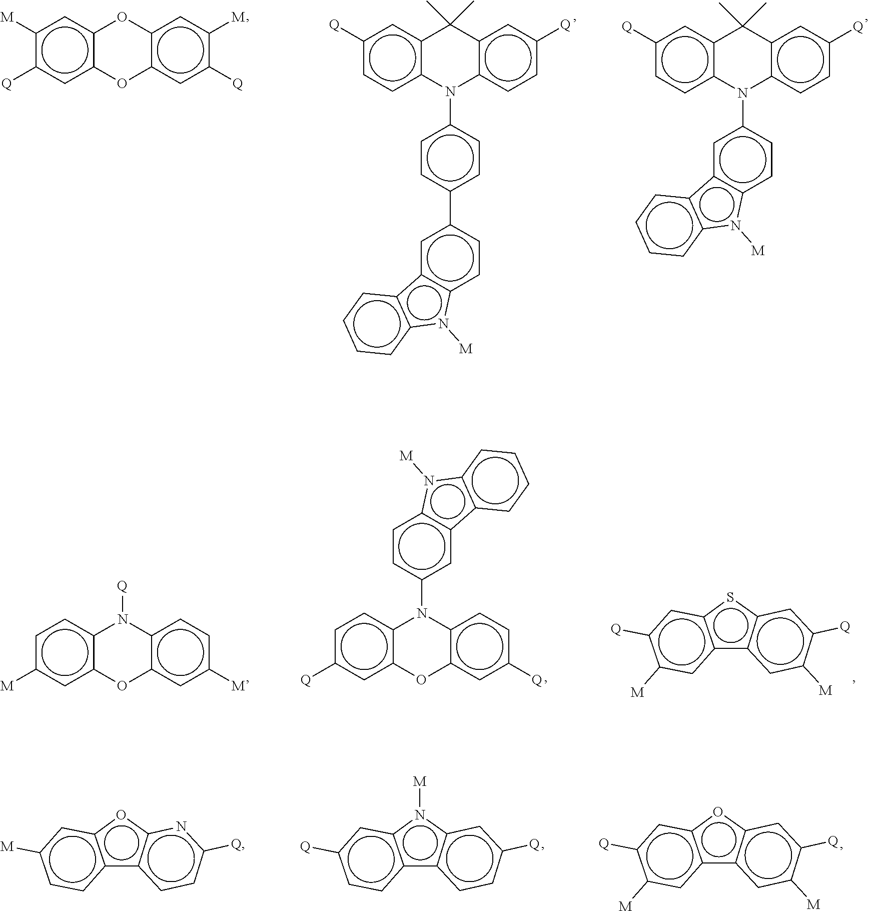

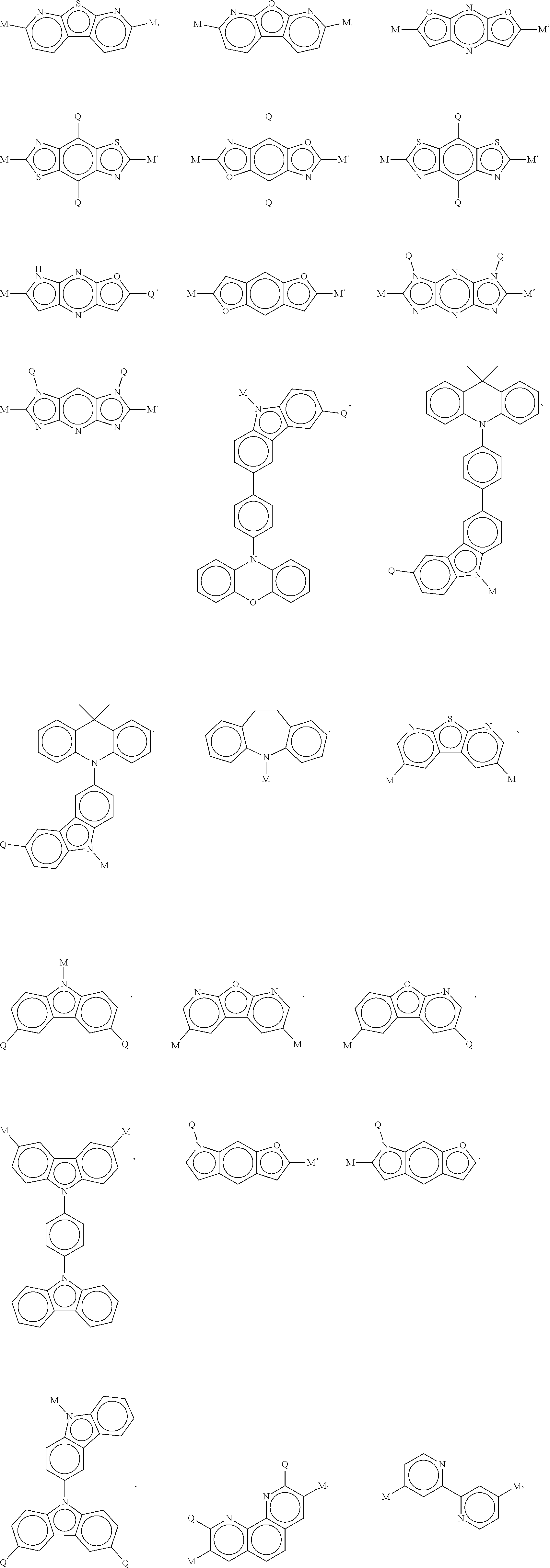

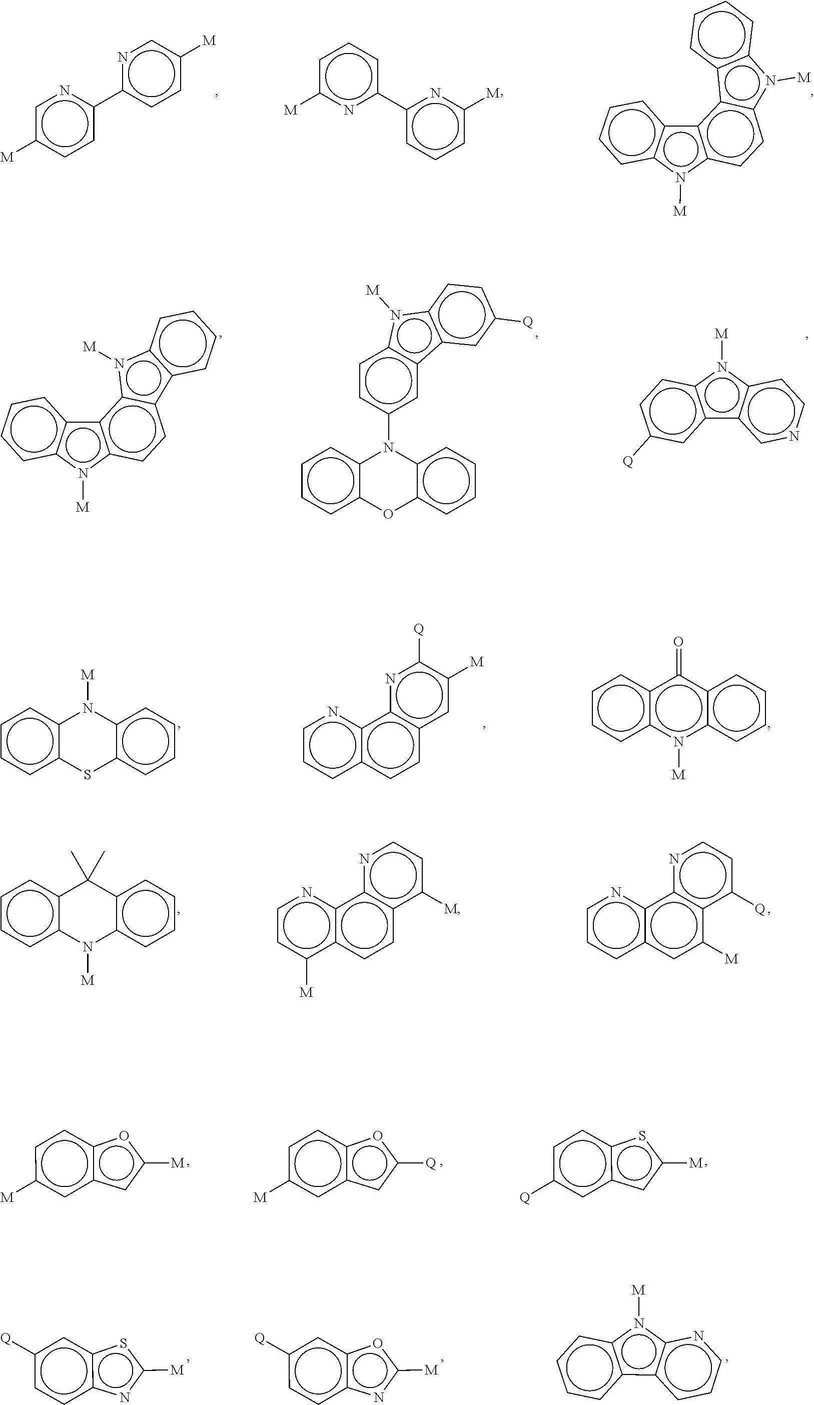

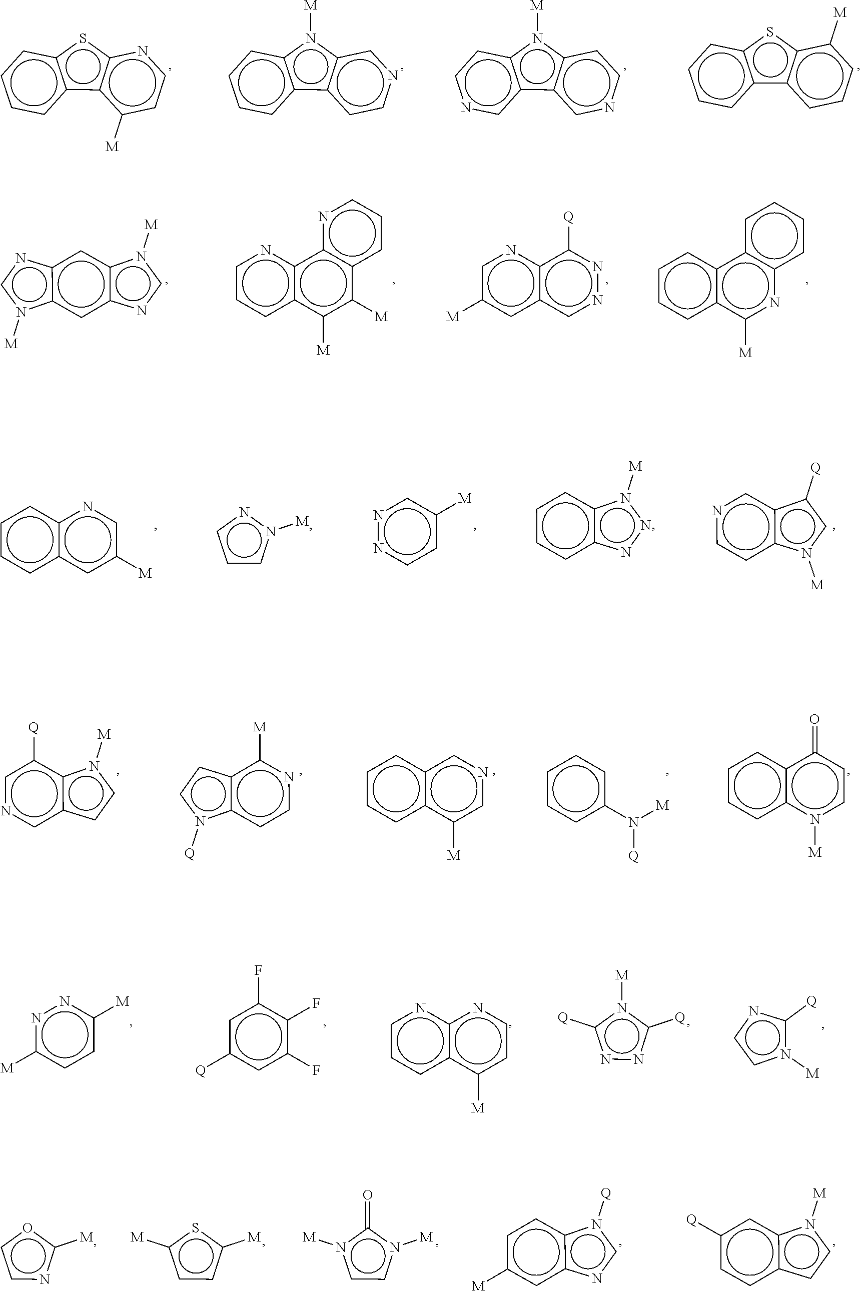

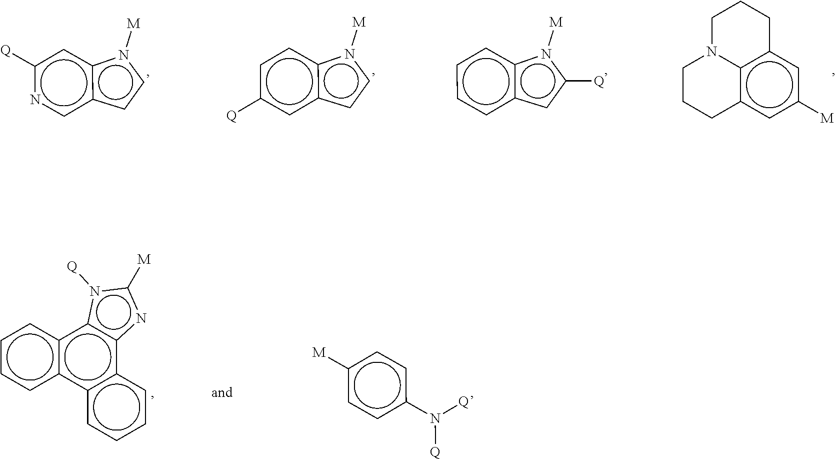

wherein, within each molecule:

Q is independently selected from the group consisting of the moiety A, a moiety B₀₋₂-A, H, C₁-C₃ alkyl, C₆-C₁₈ aryl, oxo, (5-20 atom) heteroaryl, and —N(C₆-C₁₈ aryl)₂,

M is independently selected from the group consisting of the moiety A, a moiety B₀₋₂-A, H, C₁-C₃ alkyl, C₆-C₁₈ aryl, oxo, (5-20 atom) heteroaryl, and —N(C₆-C₁₈ aryl)₂,

at least one of Q and M is the moiety B₀₋₂-A,

all groups Q are the same and all groups M are the same, and

each group Q is the same or different from any group M, and wherein the moieties A and B are defined above with respect to the first, second, and third aspects of the present invention.

In an example embodiment of the ninth aspect of the present invention, the moiety A, for each occurrence independently, is selected from List A4.

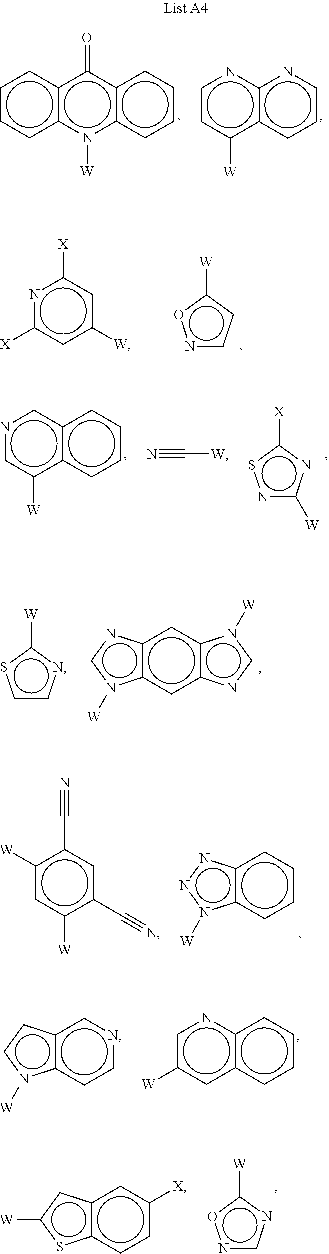

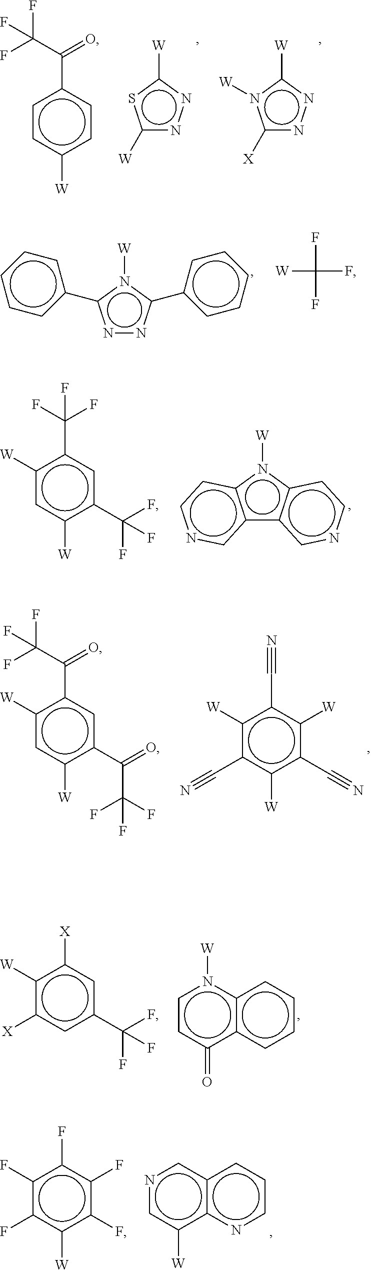

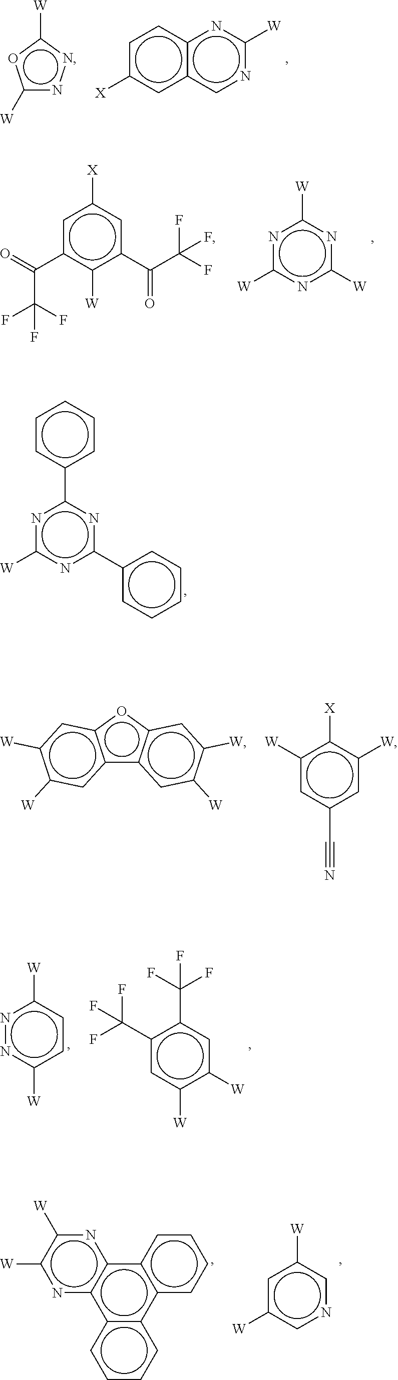

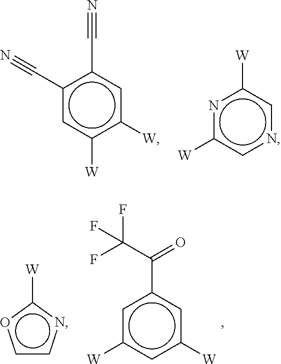

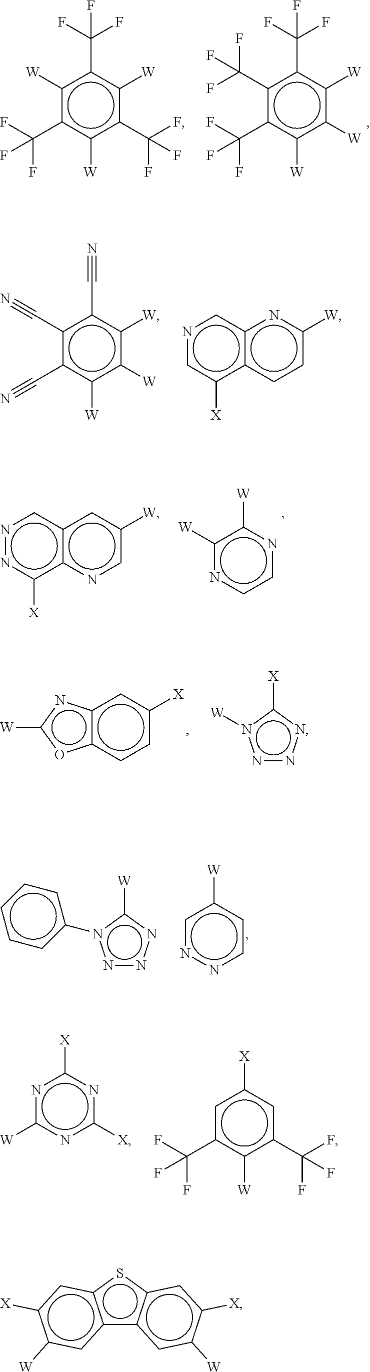

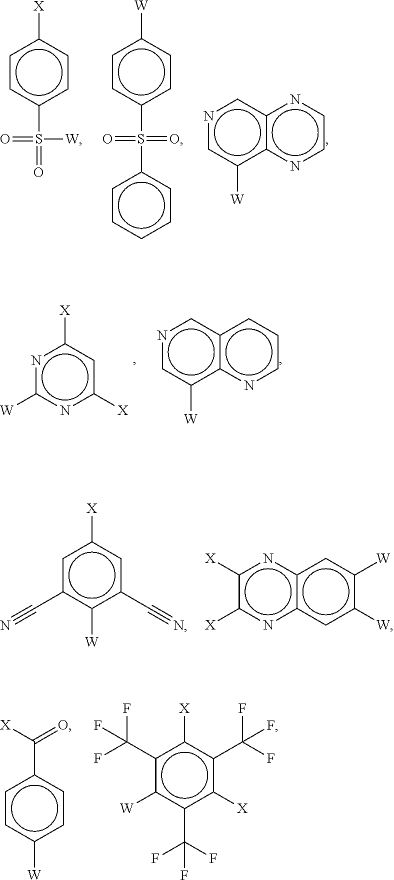

wherein, within each molecule:

W is the moiety D or a moiety B₀₋₂-D and each X is the moiety D or the moiety B₀₋₂-D,

all groups W are the same and all groups X are the same, and

each group W is the same or different from any group X, and wherein the moieties D and B are defined above with respect to the first, second, and third aspects of the present invention.

In an example embodiment of the tenth aspect of the present invention, the moiety A, for each occurrence independently, can be selected from List A4, List A5, or both.

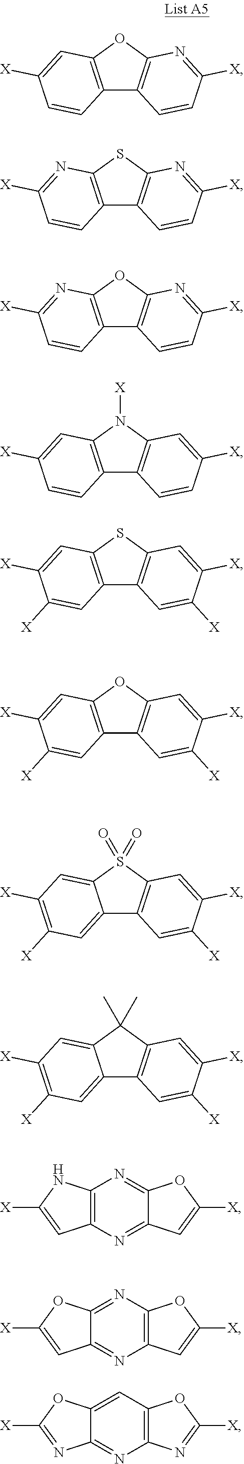

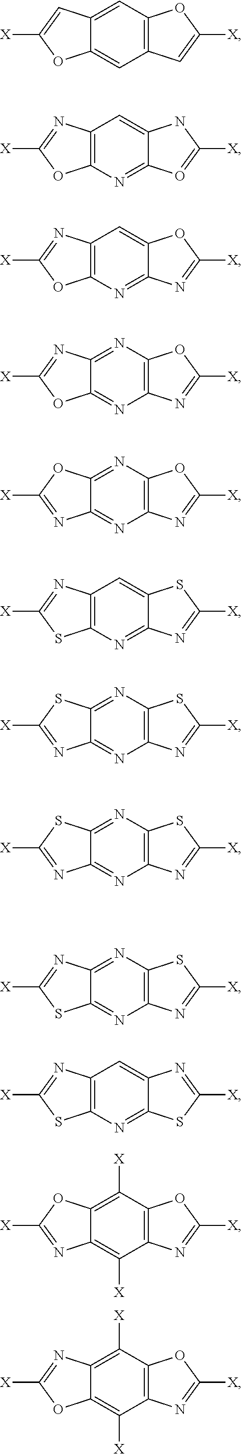

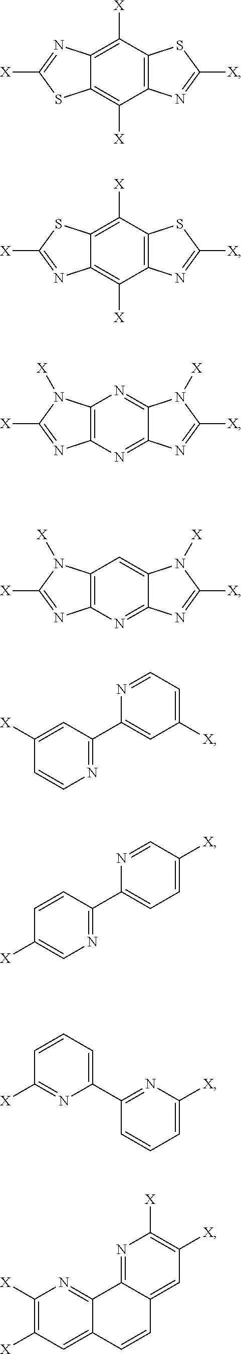

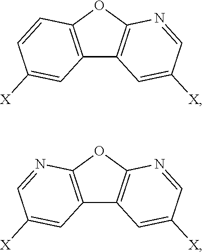

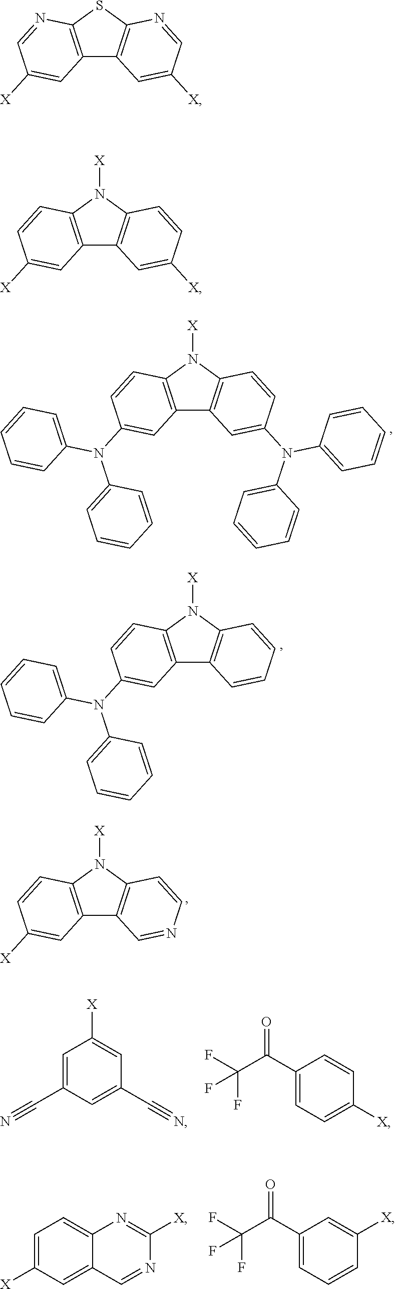

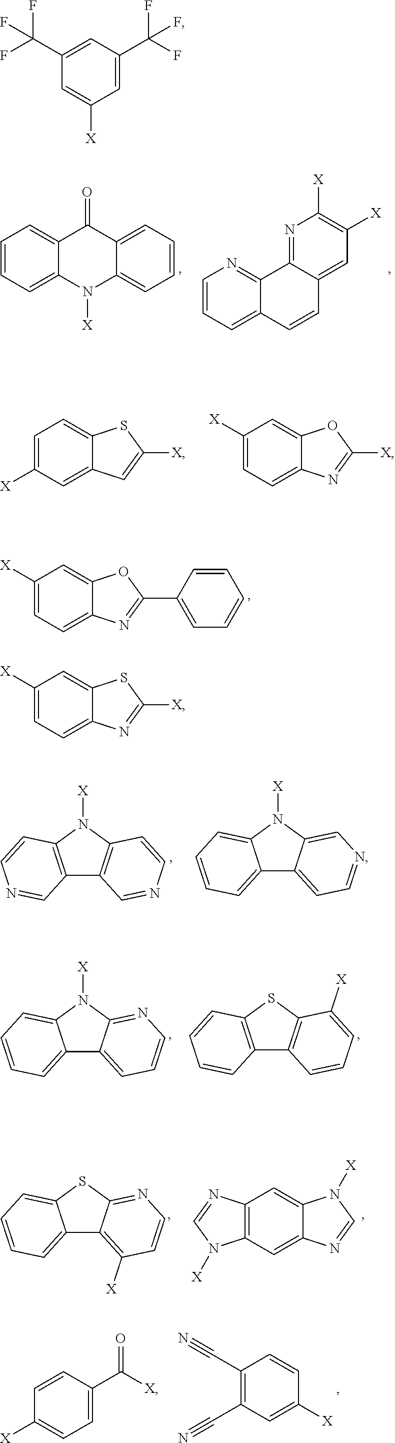

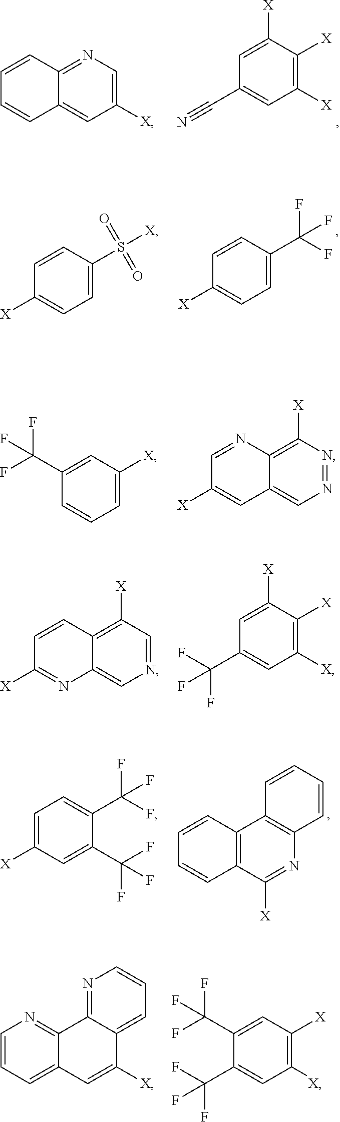

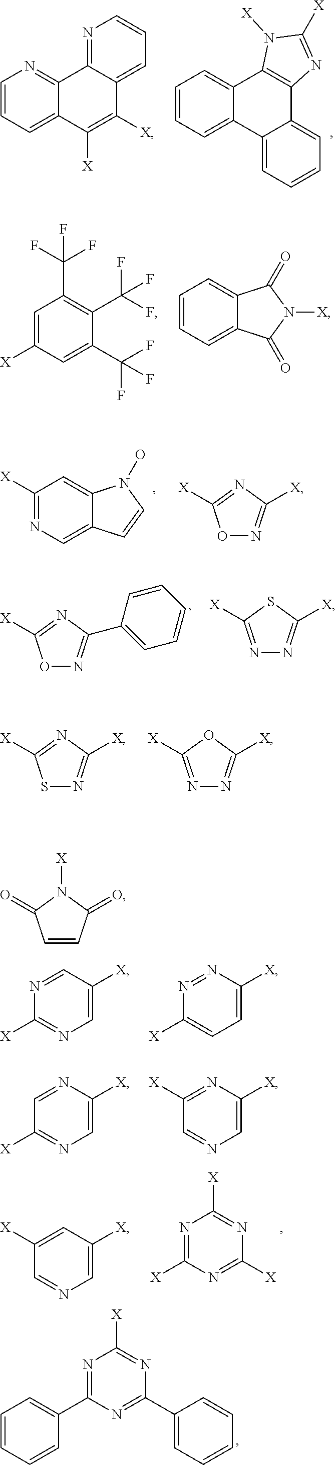

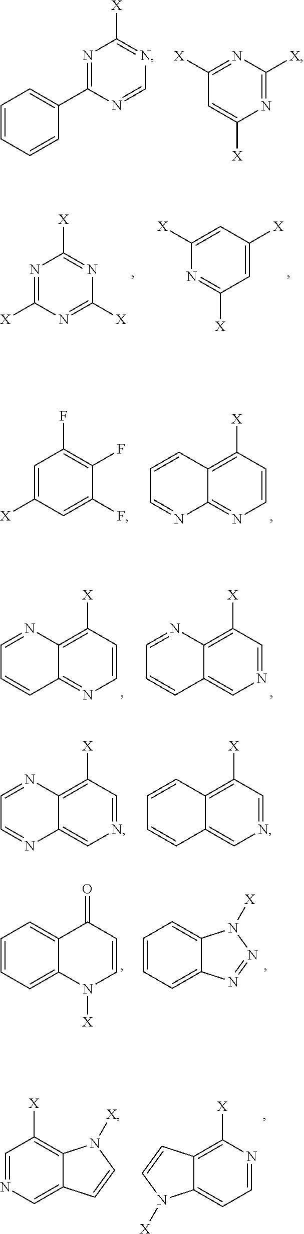

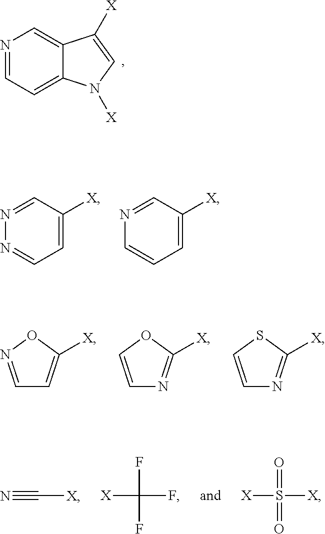

wherein, within each molecule:

X is selected from the group consisting of the moiety D, a moiety B₀₋₂-D, H, C₁-C₃ alkyl, C₆-C₁₈ aryl, oxo, C₁-C₃ haloalkyl, —CN, —CF₃, —C(O)C₁-C₃ haloalkyl, —F, and —S(O₂)H, and wherein the moieties D and B are defined above with respect to the first, second, and third aspects of the present invention.

In an example embodiment of the tenth and eleventh aspects of the present invention, the moiety A, for each occurrence independently, can be selected from List A4, List A5, List A6, or any combination thereof.

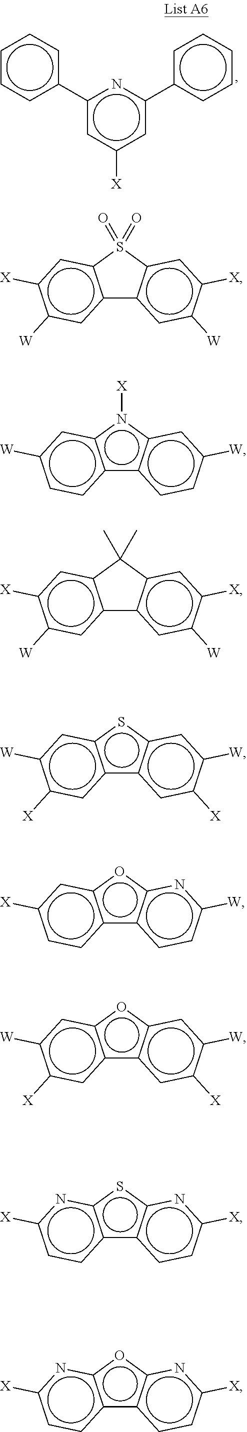

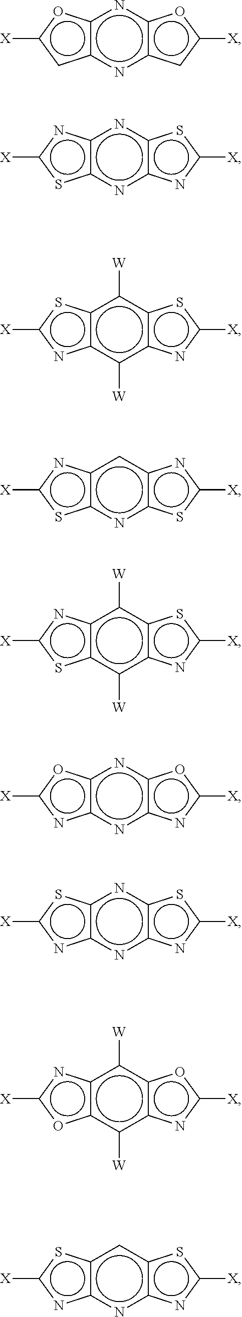

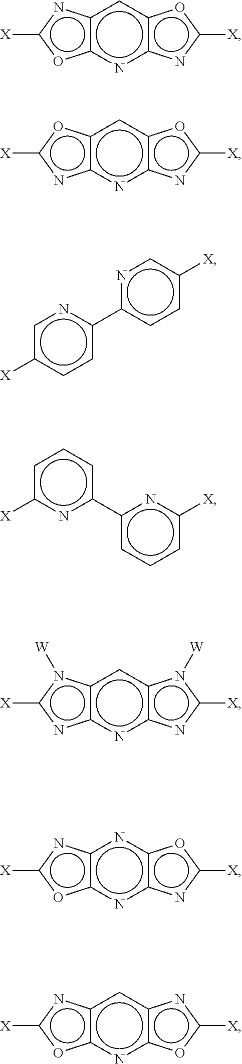

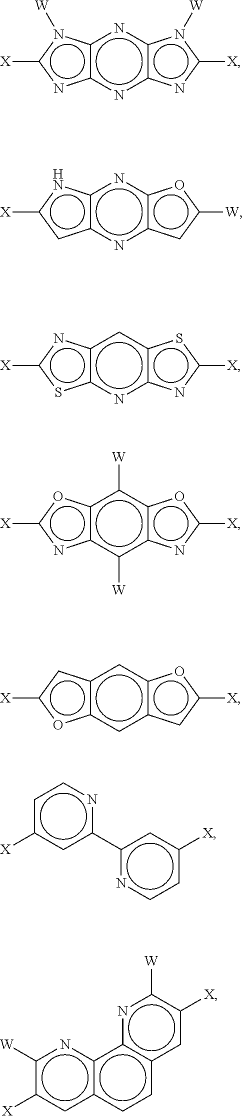

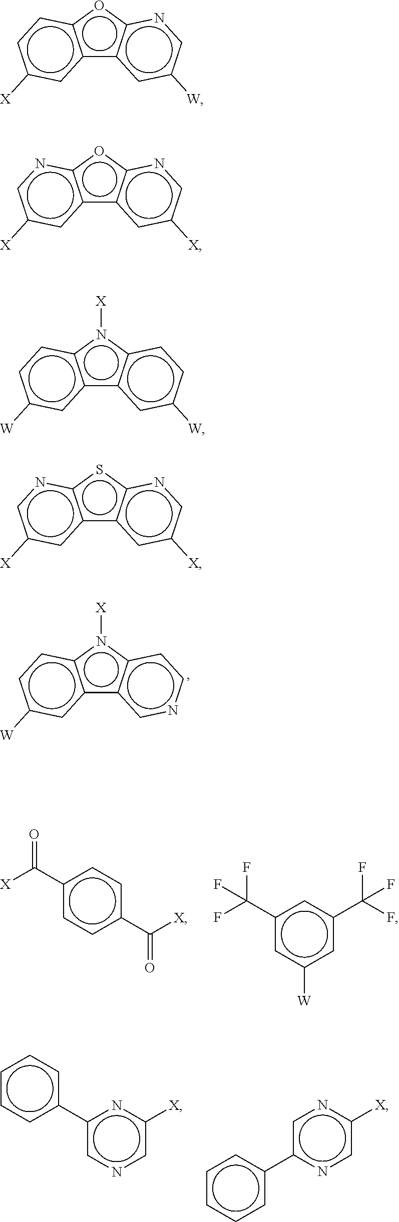

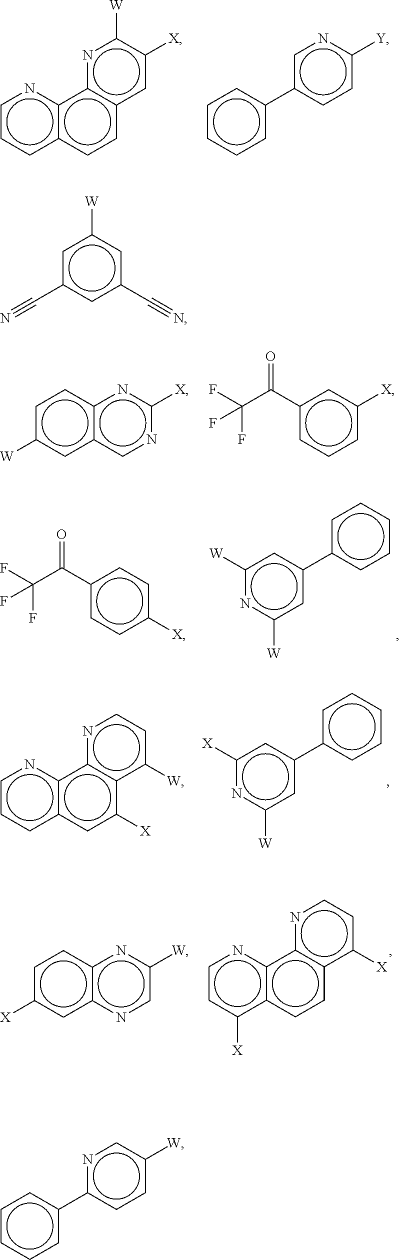

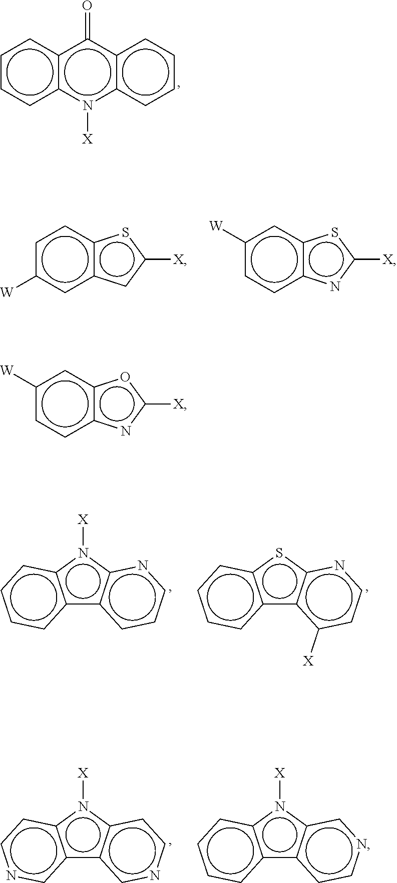

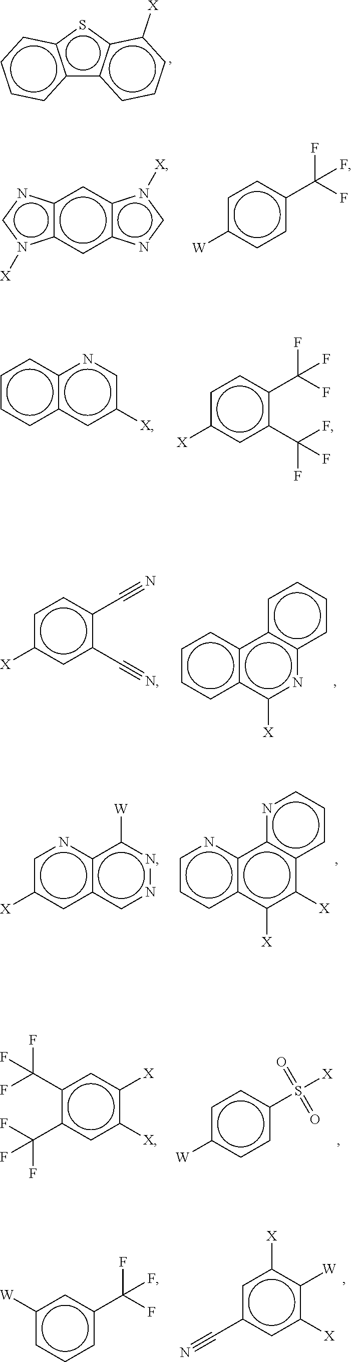

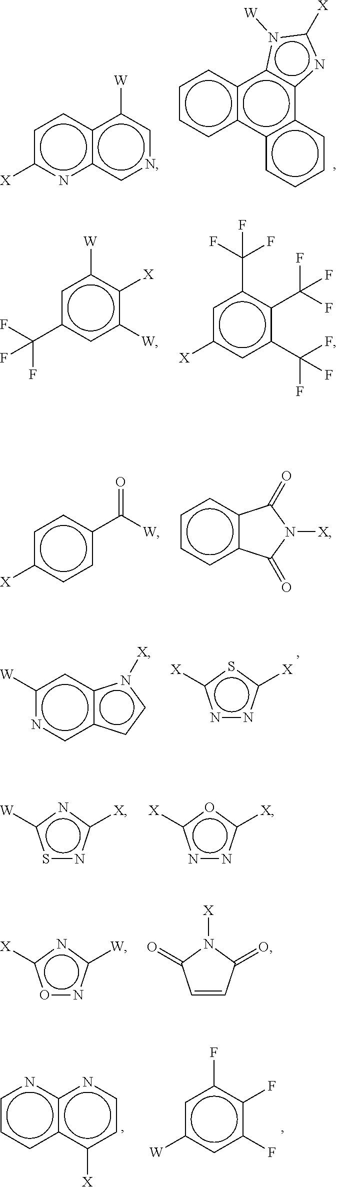

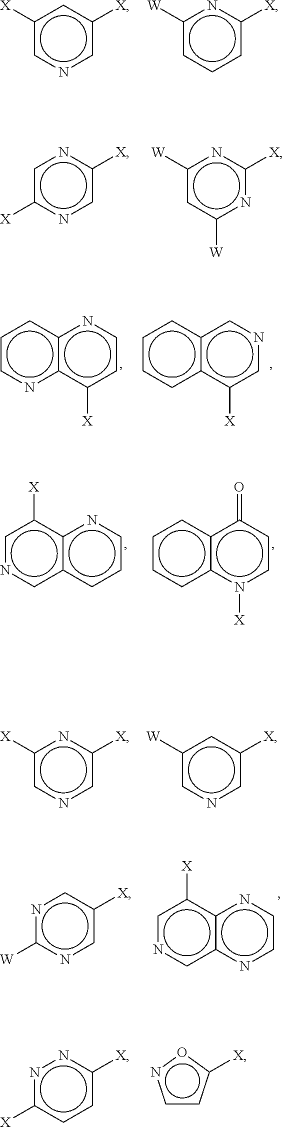

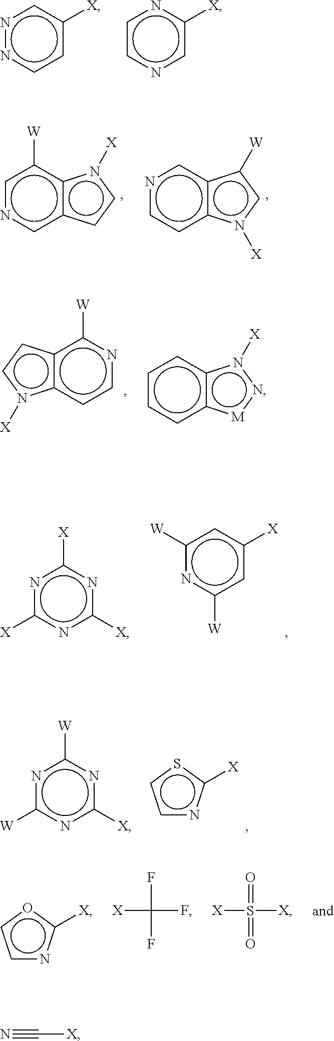

wherein, within each molecule:

X is selected from the group consisting of a moiety B₀₋₂-D, H, C₁-C₃ alkyl, C₆-C₁₈ aryl, oxo, C₁-C₃ haloalkyl, —CN, —CF₃, —C(O)C₁-C₃ haloalkyl, —F, and —S(O₂)H,

W is selected from the group consisting of the moiety B₀₋₂-D, H, C₁-C₃ alkyl, C₁-C₃ acylalkyl, C₆-C₁₈ aryl, oxo, C₁-C₃ haloalkyl, —CN, —CF₃, —C(O)C₁-C₃ haloalkyl, —F, and —S(O₂)H,

at least one of W and X is the moiety B₀₋₂-D,

all groups W are the same and all groups X are the same, and

each group W is the same or different from any group X, and wherein the moieties D and B are defined above with respect to the first, second, and third aspects of the present invention.

In an example embodiment of the twelfth aspect of the present invention, the moiety B, for each occurrence independently, is selected from List B3.

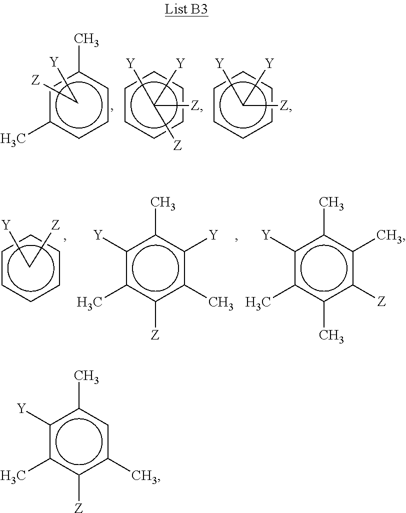

wherein, within each molecule:

Y is the moiety A, the moiety B₀₋₁-A, the moiety D, or the moiety B₀₋₁-D and each Z is the moiety A, a moiety B₀₋₁-A, the moiety D, or a moiety B₀₋₁-D,

within a given molecule all groups Y are the same and all groups Z are the same, and

each group Y is the same or different from any group Z, and wherein the moieties A and D are defined above with respect to the first, second, and third aspects of the present invention.

In an example embodiment of the thirteenth aspect of the present invention, the moiety B, can also be selected from List B3, List B4, or both.

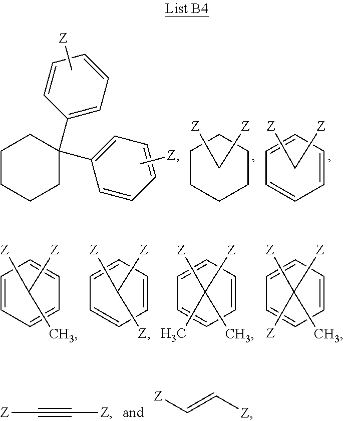

wherein, within each molecule:

Z is independently selected from the group consisting of the moiety A, a moiety B₀₋₁-A, the moiety D, a moiety B₀₋₁-D, H, C₁-C₃ alkyl, and C₆-C₁₈ aryl, and wherein the moieties A and D are defined above with respect to the first, second, and third aspects of the present invention.

In an example embodiment of the twelfth and thirteenth aspects of the present invention, the moiety B, can also be selected from List B3, List B4, List B5, or any combination thereof.

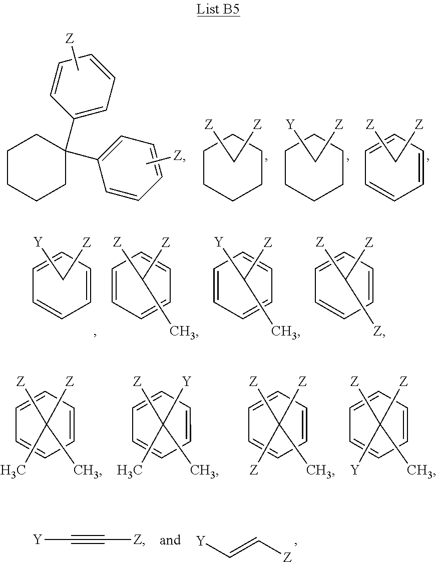

wherein, within each molecule:

Z is the moiety A, a moiety B₀₋₁-A, the moiety D, a moiety B₀₋₁-D, H, C₁-C₃ alkyl, or C₆-C₁₈ aryl,

Y is the moiety A, the moiety B₀₋₁-A, the moiety D, or the moiety B₀₋₁-D and each Z is the moiety A, a moiety B₀₋₁-A, the moiety D, or a moiety B₀₋₁-D,

within a given molecule all groups Y are the same and all groups Z are the same, and

each group Y is the same or different from any group Z, and wherein the moieties A and D are defined above with respect to the first, second, and third aspects of the present invention.

In an example embodiment of the twelfth aspect of the present invention, the moiety B, for each occurrence independently, is selected from List B3, List B4, List B5, List B6, or any combination thereof.

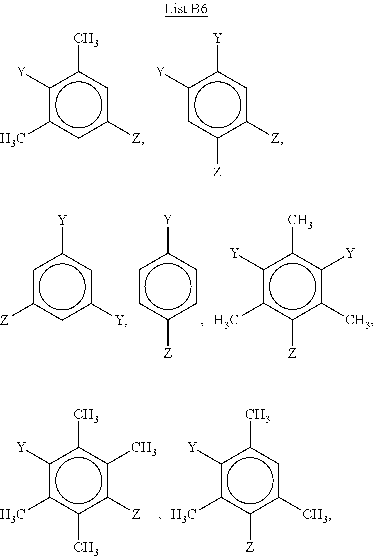

wherein, within each molecule:

Y is the moiety A, the moiety B₀₋₁-A, the moiety D, or the moiety B₀₋₁-D and each Z is the moiety A, a moiety B₀₋₁-A, the moiety D, or a moiety B₀₋₁-D,

within a given molecule all groups Y are the same and all groups Z are the same, and

each group Y is the same or different from any group Z, and wherein the moieties A and D are defined above with respect to the first, second, and third aspects of the present invention.

In an example embodiment of the thirteenth aspect of the present invention, the moiety B, for each occurrence independently, is selected from List B3, List B4, List B5, List B6, List B7, or any combination thereof.

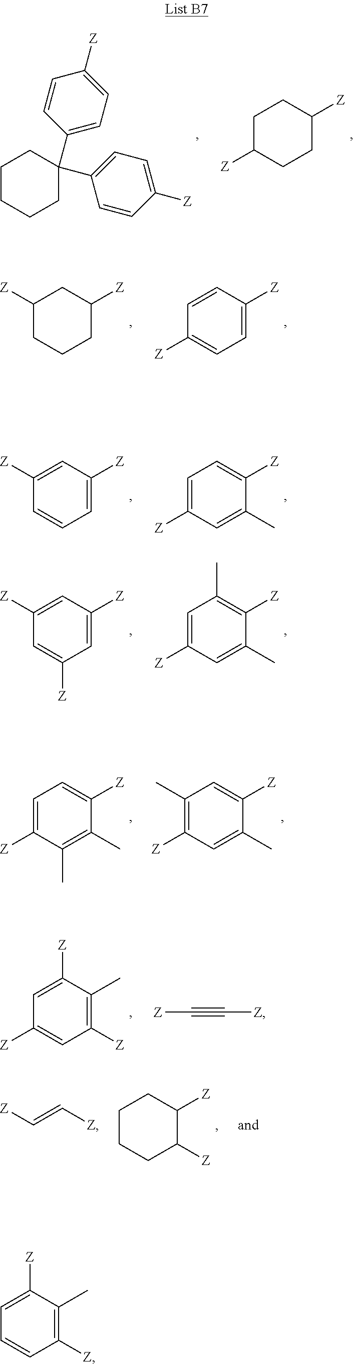

wherein, within each molecule:

Z is the moiety A, the moiety B₀₋₁-A, the moiety D, the moiety B₀₋₁-D, H, C₁-C₃ alkyl, or C₆-C₁₈ aryl, and wherein the moieties A and D are defined above with respect to the first, second, and third aspects of the present invention.

In an example embodiment of the twelfth and thirteenth aspects of the present invention, the moiety B, for each occurrence independently, is selected from List B3, List B4, List B5, List B6, List B7, List B8 or any combination thereof.

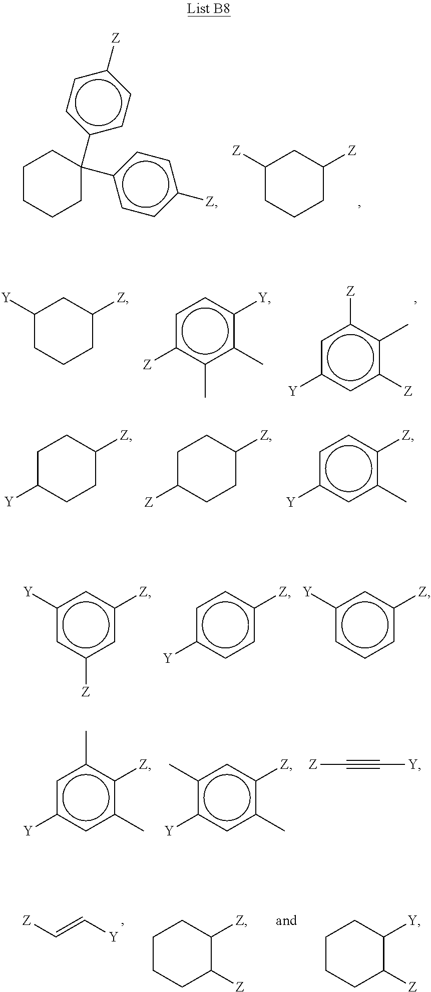

wherein, within each molecule:

Z is the moiety A, the moiety B₀₋₁-A, the moiety D, the moiety B₀₋₁-D, H, C₁-C₃ alkyl, or C₆-C₁₈ aryl,

Y is the moiety A, the moiety B₀₋₁-A, the moiety D, the moiety B₀₋₁-D, H, C₁-C₃ alkyl, or C₆-C₁₈ aryl,

within a given molecule all groups Y are the same and all groups Z are the same, and

each group Y is the same or different from any group Z, and wherein the moieties A and D are defined above with respect to the first, second, and third aspects of the present invention.

In an example embodiment of any one of the first through thirteenth aspects of the present invention described above, the moiety D is optionally substituted with one or more substituents each independently selected from C₁-C₃ alkyl, C₆-C₁₈ aryl, or oxo, and wherein A, B, and D are defined above with respect to the first or second aspects of the present invention.

In an example embodiment of any one of the first through thirteenth aspects of the present invention described above, the moiety D is optionally substituted with one or more substituents each independently selected from (5-20 atom) heteroaryl or —N(C₆-C₁₈aryl)₂, and wherein A, B, and D are defined above with respect to the first or second aspects of the present invention.

In an example embodiment of any one of the first through thirteenth aspects of the present invention described above, the moiety D is optionally substituted with one or more substituents each independently selected from C₁-C₃ alkyl, C₆-C₁₈ aryl, oxo, (5-20 atom) heteroaryl, or —N(C₆-C₁₈aryl)₂, and wherein A, B, and D are defined above with respect to the first or second aspects of the present invention.

In an example embodiment of any one of the first through thirteenth aspects of the present invention described above, the moiety A is optionally substituted with one or more substituents each independently selected from C₁-C₃ alkyl, C₆-C₁₈ aryl, oxo, C₁-C₃ haloalkyl, —CN, —CF₃, —C(O)C₁-C₃ haloalkyl, —F, and —S(O₂)H, and wherein A, B, and D are defined above with respect to the first or second aspects of the present invention.

In an example embodiment of any one of the first through thirteenth aspects of the present invention described above, the moiety B is optionally substituted with C₁-C₃ alkyl, and wherein A, B, and D are defined above with respect to the first or second aspects of the present invention.

In an example embodiment of any one of the first through thirteenth aspects of the present invention described above, the moiety B is optionally substituted with C₆-C₁₈ aryl, and wherein A, B, and D are defined above with respect to the first or second aspects of the present invention.

In an example embodiment of any one of the first through thirteenth aspects of the present invention described above, the moiety B is optionally substituted with one or more substituents each independently selected from C₁-C₃ alkyl or C₆-C₁₈ aryl, and wherein A, B, and D are defined above with respect to the first or second aspects of the present invention.

In a fourteenth aspect, the present invention is a molecule of one of the structural formulas represented in Tables 1-14. The carbon or heteroatom denoted by (*) in the structural formulas of Tables 1-14 are unsubstituted or substituted by a C₁-C₆ alkyl, —OH, —CN, a halo, a C₆-C₁₂ aryl, a 5-20 atom heteroaryl, —N(R¹⁹)₂ or —N(R²⁰)₂, wherein each R¹⁹, independently, is H or a C₁-C₆ alkyl, or a C₅-C₁₂ cycloalkyl, and wherein each R²⁰, independently, is H or a C₆-C₁₈ aryl.

In the fifteenth aspect of the present invention, the molecule is not represented by the structural formulas B4, J68, J79, K39, K55, K57, K100, K177, or N6 in Tables 1-14.

In an example embodiment of the fifteenth aspect, the present invention is a molecule selected from Table 15.

In a sixteenth aspect, the present invention is a molecule represented by structural formulas (II)-(XI):

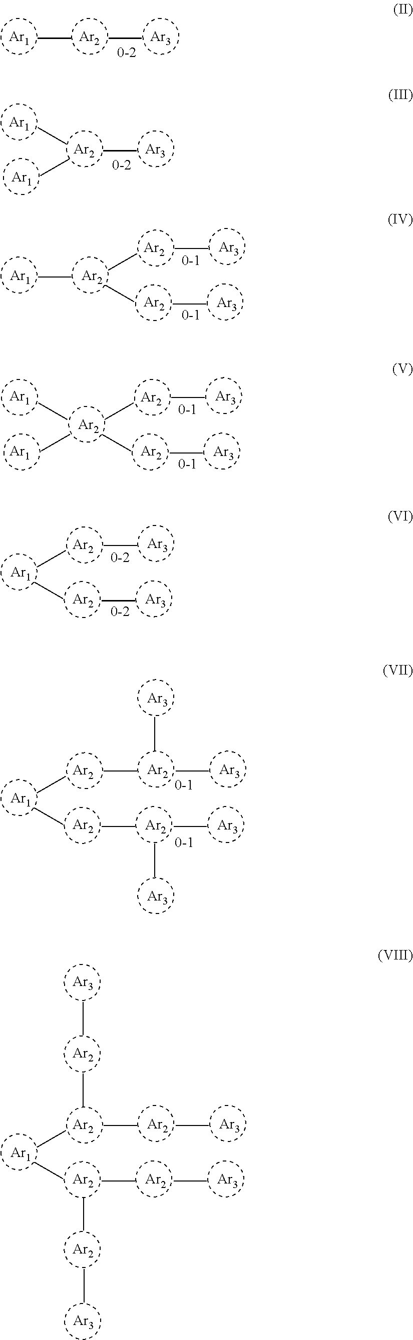

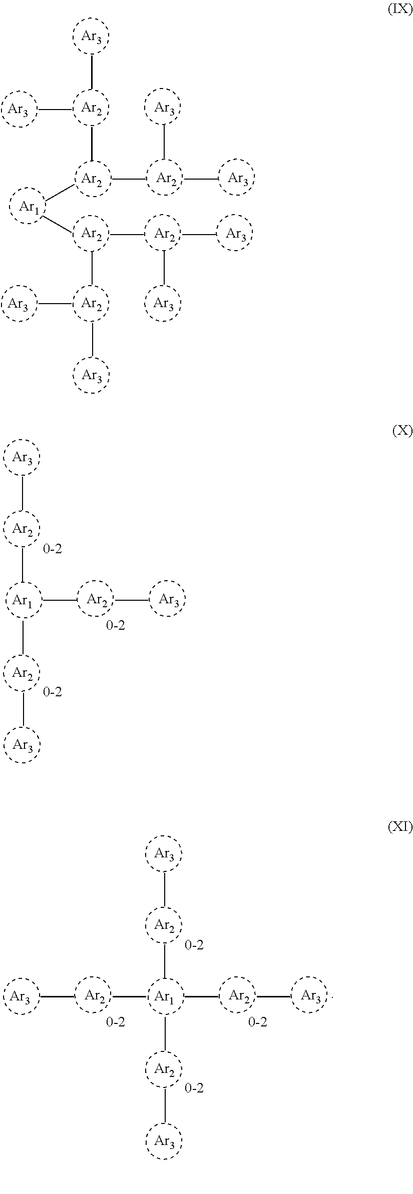

In structural formulas (II)-(XI), Ar₁ and Ar₃, for each occurrence independently, are selected from List M1.

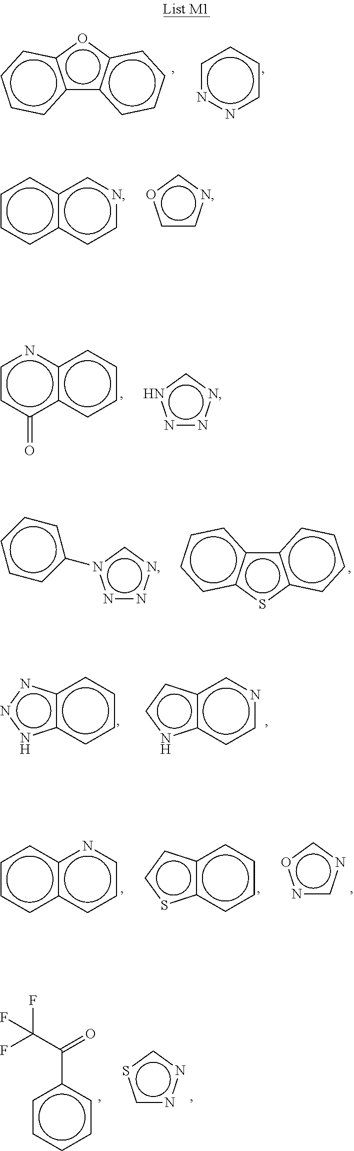

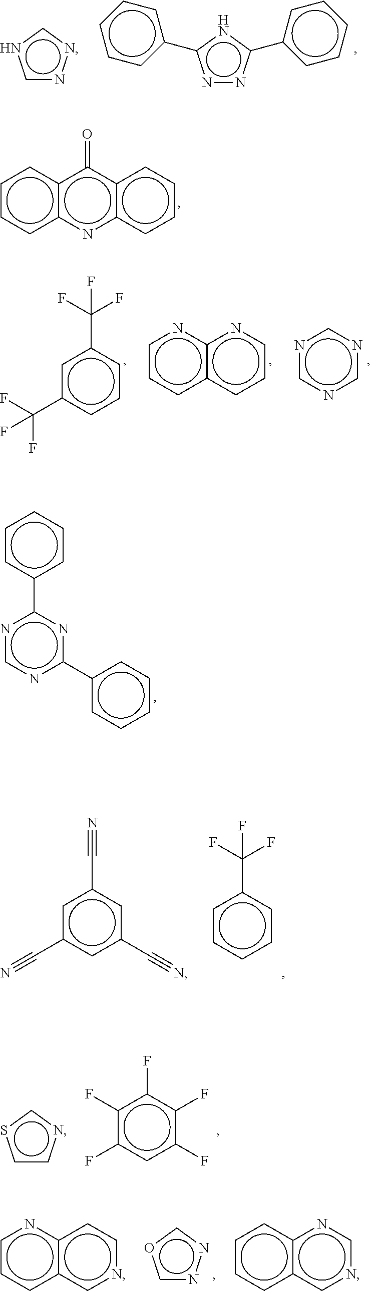

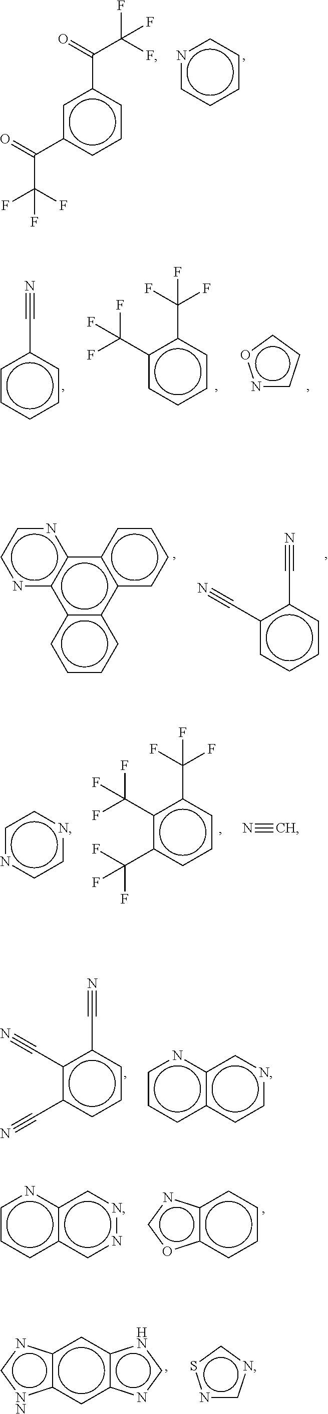

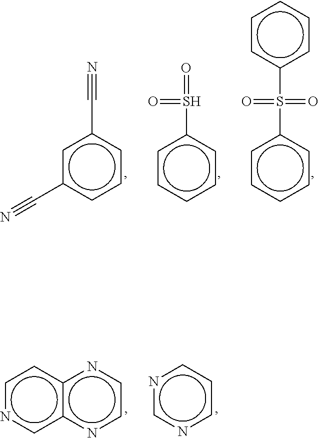

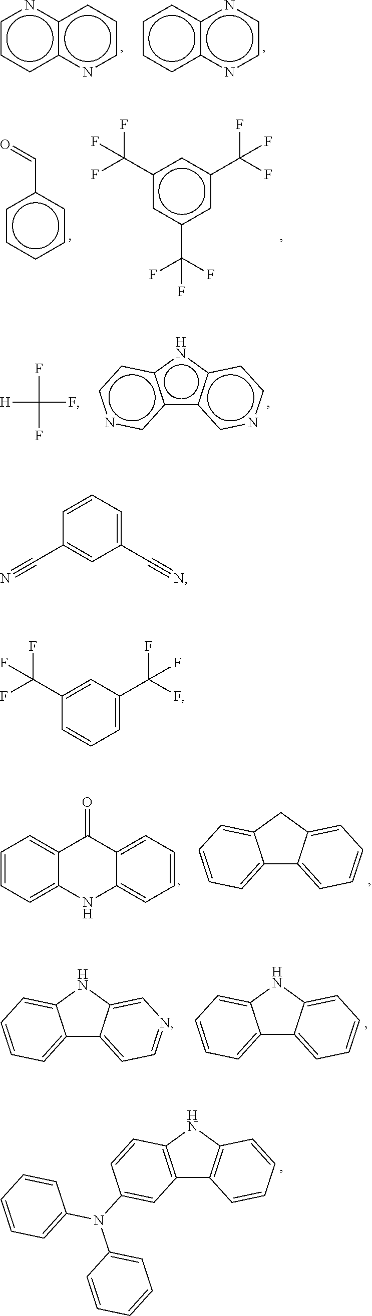

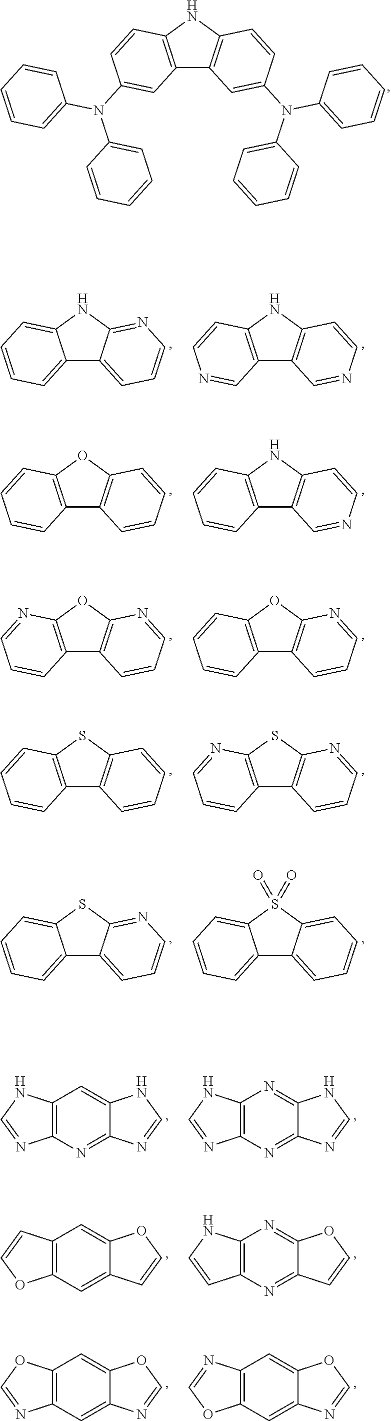

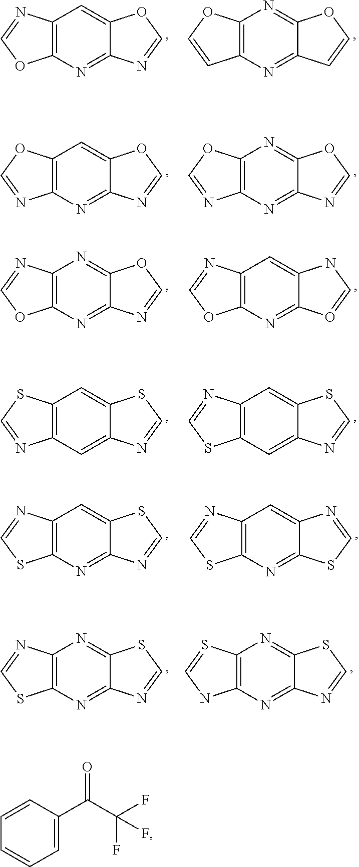

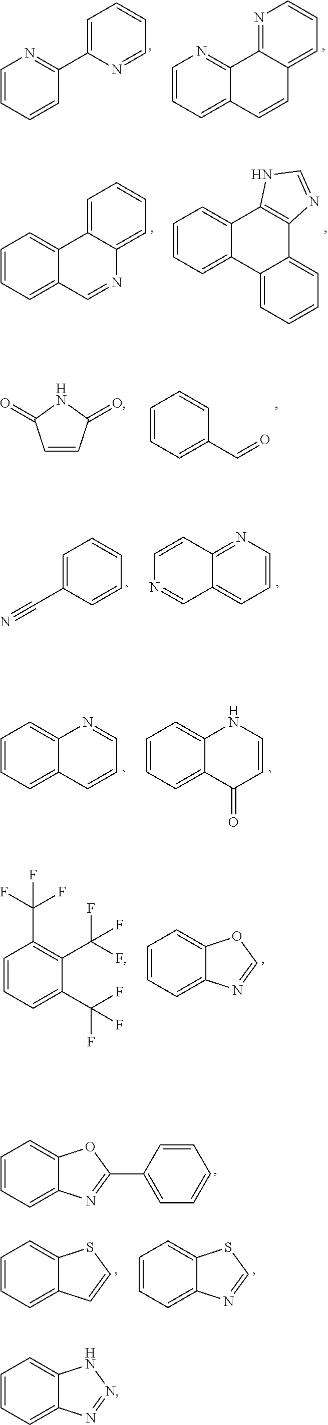

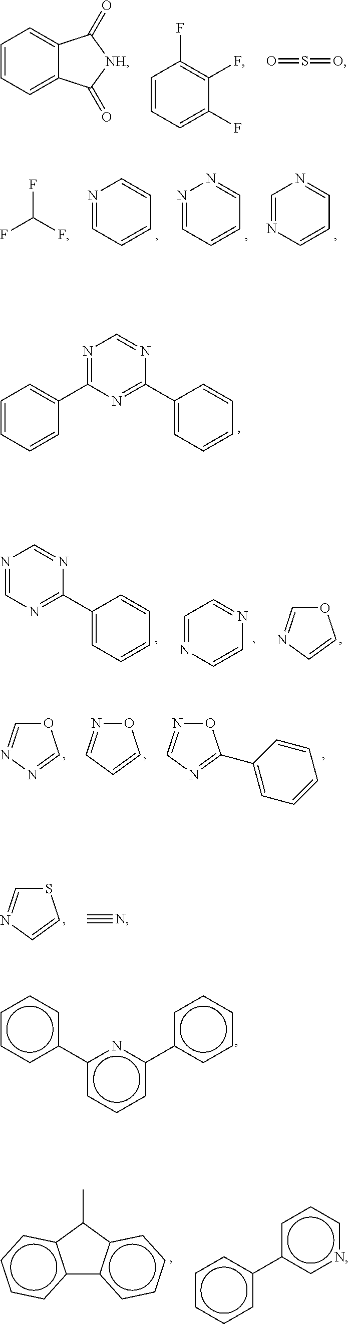

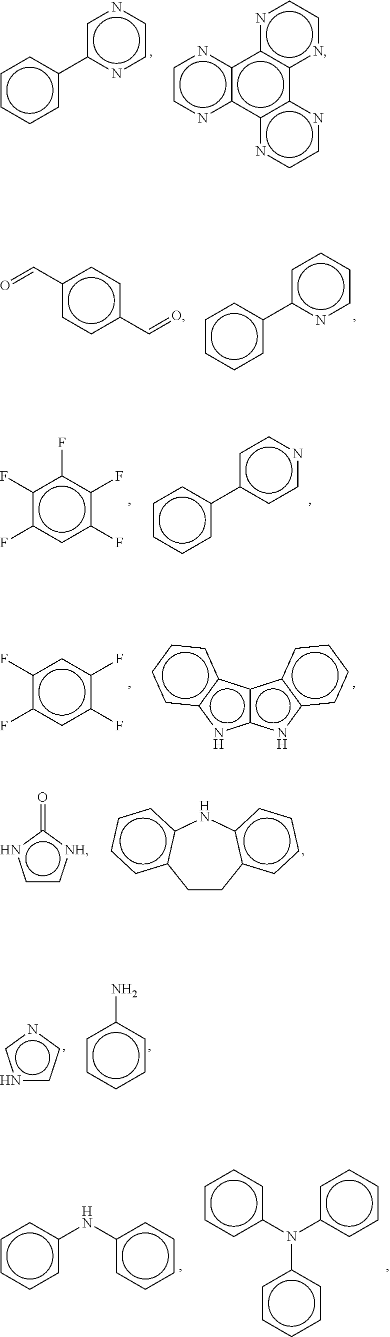

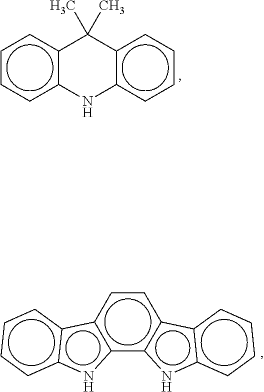

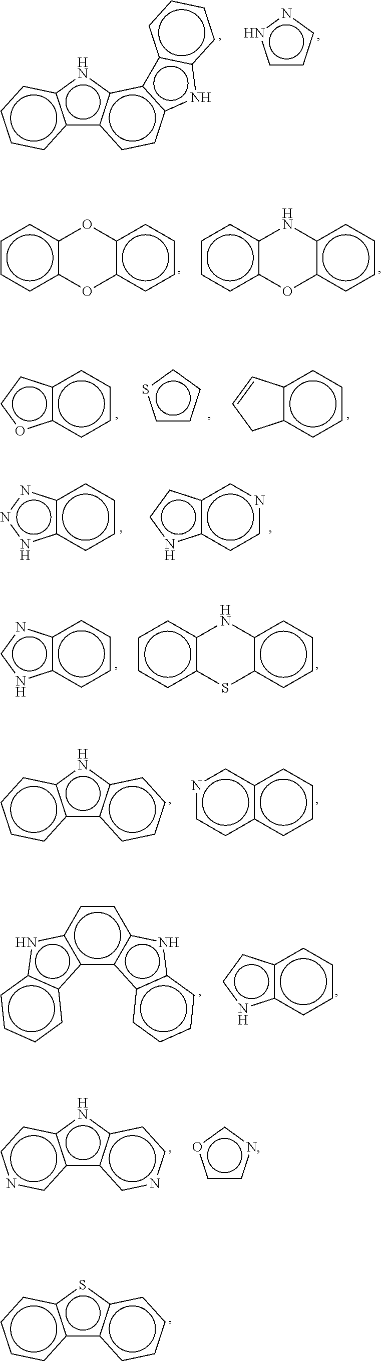

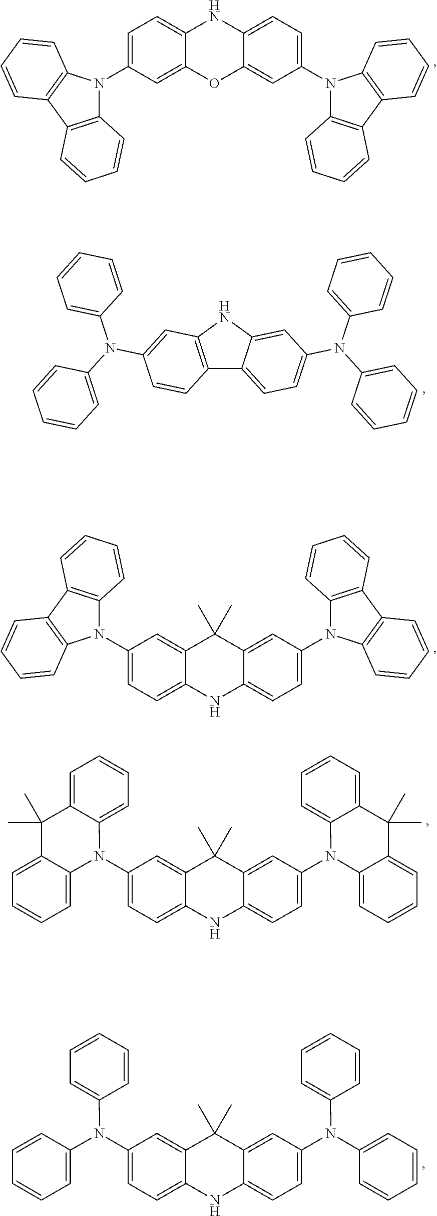

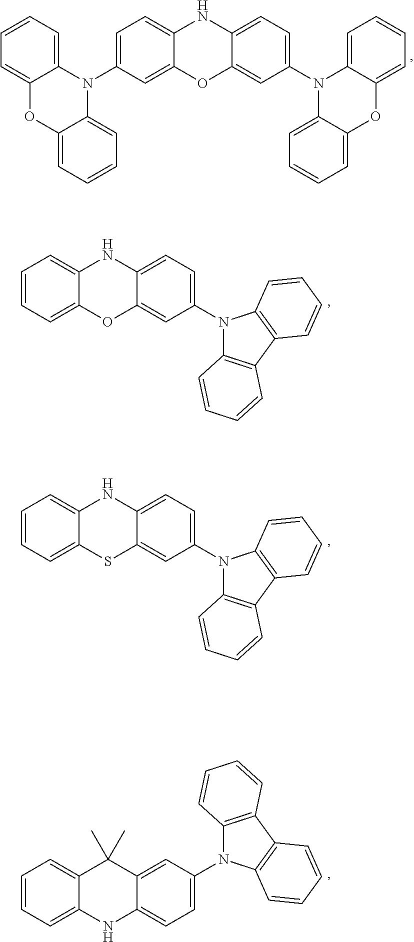 [SMILES: c1ccc2c(c1)Oc1ccccc1N2c1ccc2c(c1)Oc1cc(N3c4ccccc4Oc4ccccc43)ccc1N2 ; c1ccc2c(c1)Nc1ccc(-n3c4ccccc4c4ccccc43)cc1O2 ; c1ccc2c(c1)Nc1ccc(-n3c4ccccc4c4ccccc43)cc1S2 ; CC1(C)c2ccccc2Nc2ccc(-n3c4ccccc4c4ccccc43)cc21]

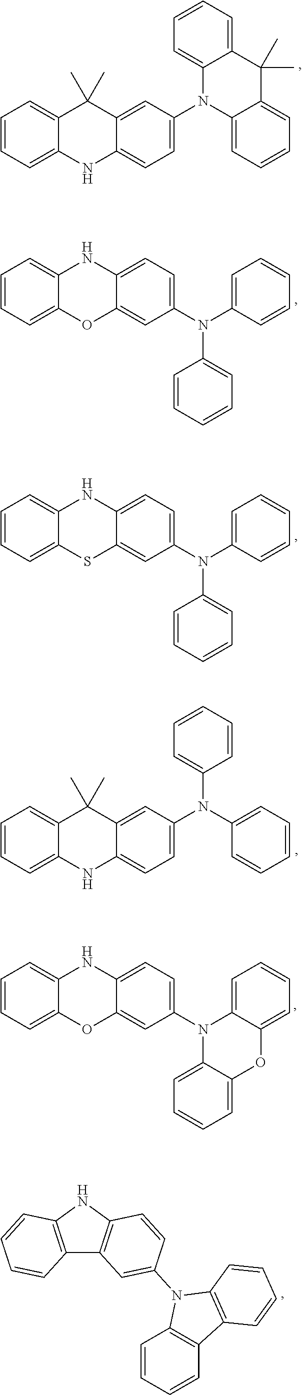

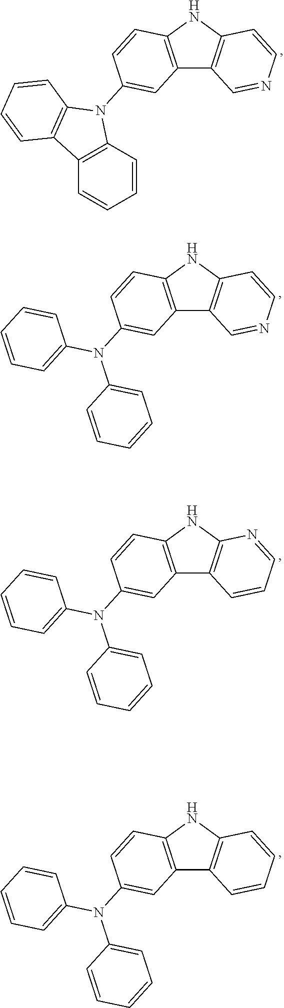 [SMILES: c1ccc2c(c1)c1ccccc1n2-c1ccc2[nH]c3ccncc3c2c1 ; c1ccc(N(c2ccccc2)c2ccc3[nH]c4ccncc4c3c2)cc1 ; c1ccc(N(c2ccccc2)c2ccc3[nH]c4ncccc4c3c2)cc1 ; c1ccc(N(c2ccccc2)c2ccc3[nH]c4ccccc4c3c2)cc1]

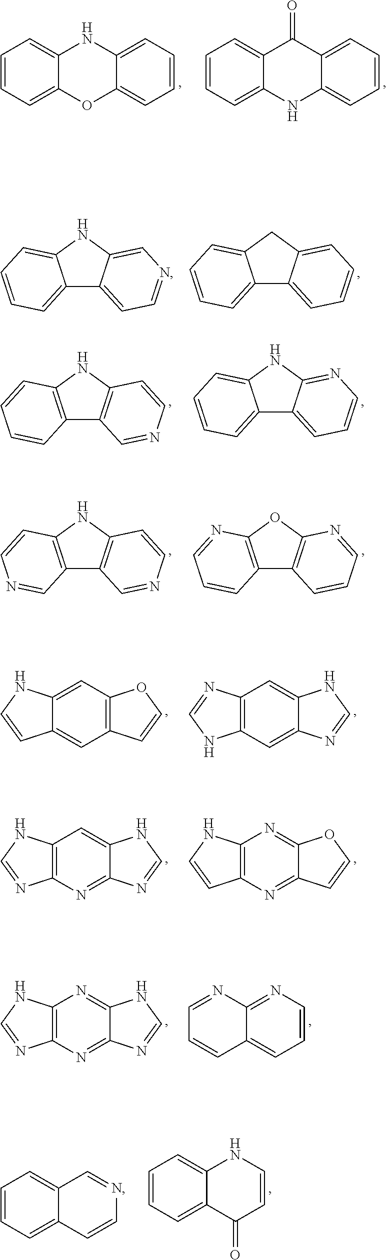

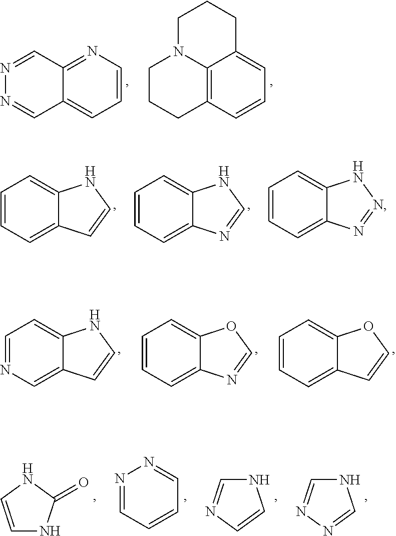

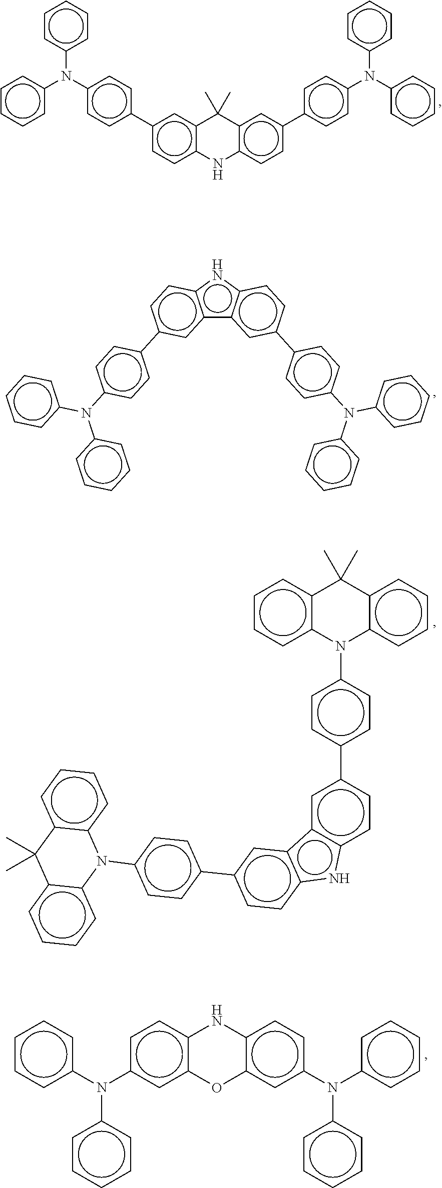

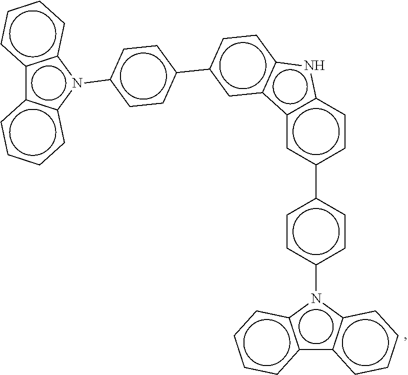

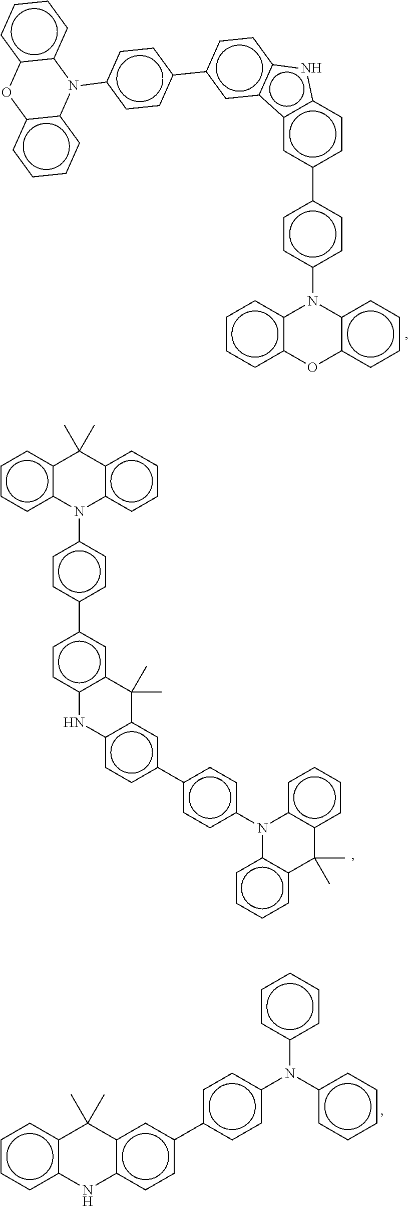

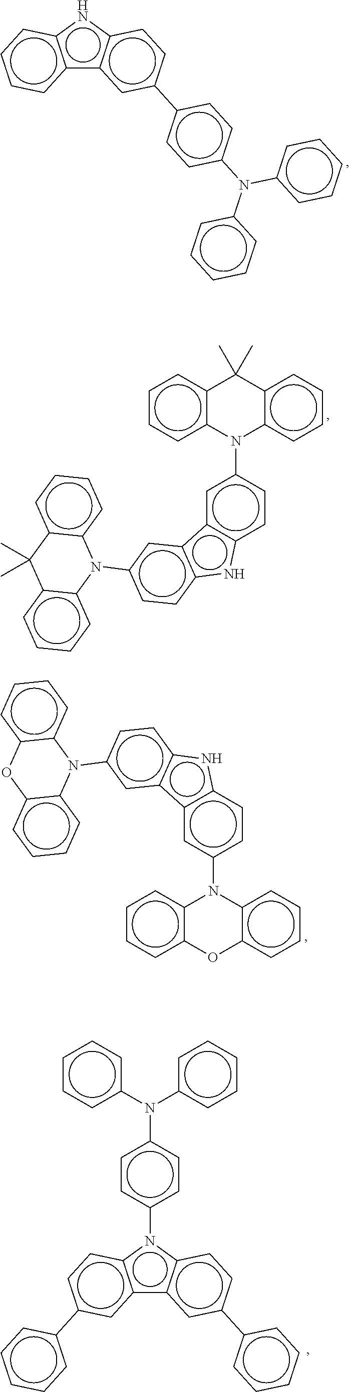

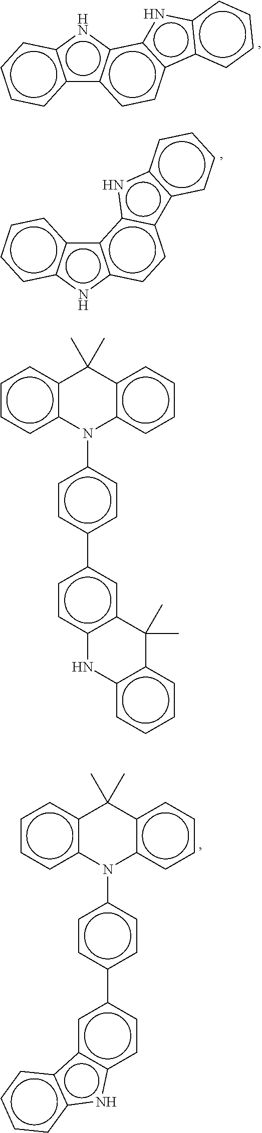

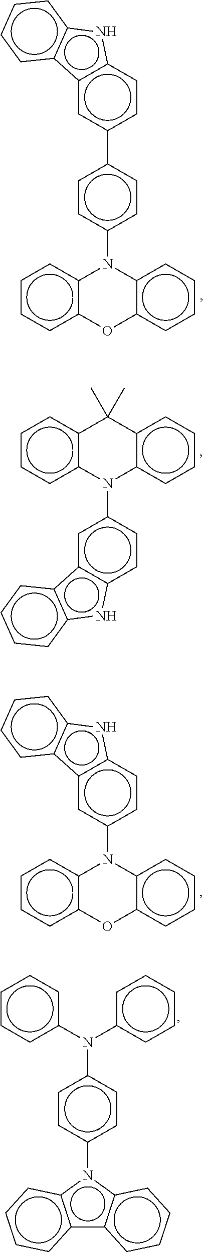

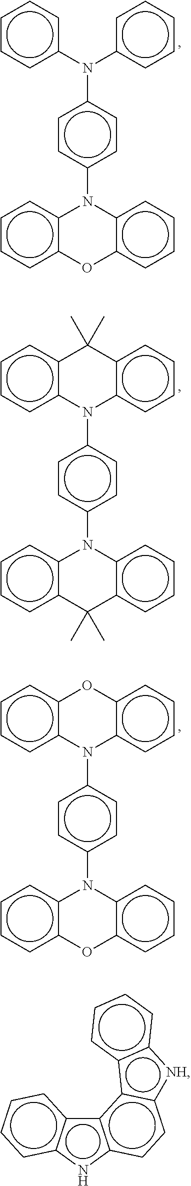

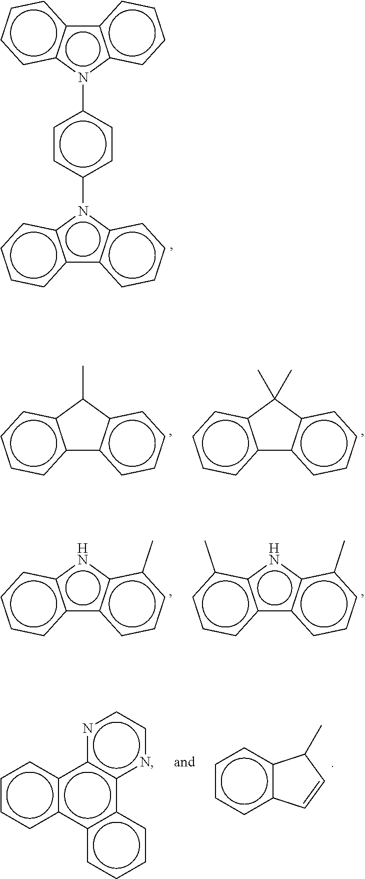

In structural formulas (II)-(XI), Ar₂, for each occurrence independently, is selected from List M2.

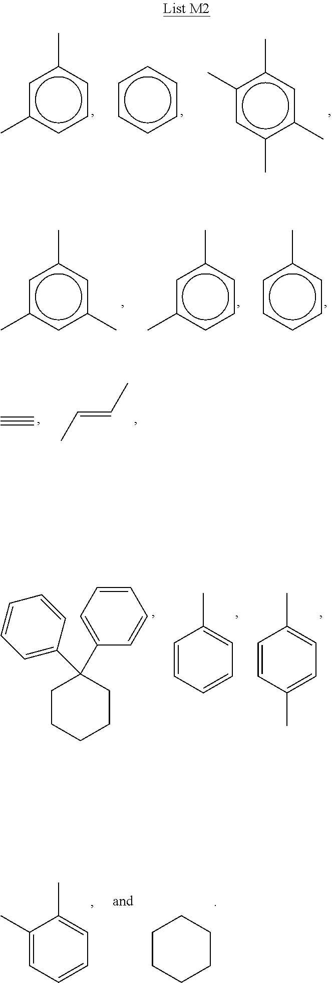

In the seventeenth aspect of the present invention, the molecule is of one of the structural formulas represented in Tables 1-14, wherein the carbon wherein the carbon or heteroatom denoted by (*) in the structural formulas represented in Tables 1-14 is unsubstituted or substituted by a C₁-C₆ alkyl, —OH, —CN, a halo, a C₆-C₁₂ aryl, a 5-20 atom heteroaryl, —N(R¹⁹)₂ or —N(R²⁰)₂, wherein each R¹⁹, independently, is H or a C₁-C₆alkyl and wherein each R²⁰, independently, is H or a C₆-C₁₈ aryl.

In the seventeenth aspect of the present invention, the molecule is not of one of the structural formulas represented by B4, J68, J79, K39, K55, K57, K100, K177, or N6 in Tables 1-14.

In an example embodiment of the seventeenth aspect of the present invention, Ar₁ and Ar₃ are different.

In an eighteenth aspect, the present invention is a molecule represented by structural formula (XII):

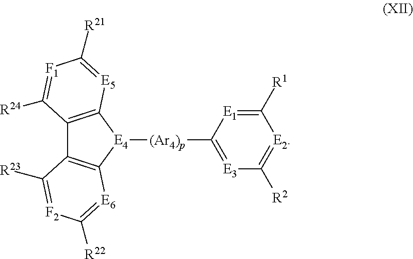

In structural formula (XII) of the present invention:

E₁, E₂, E₃, E₄, E₅, and E₆, are, each independently, CH or N.

R¹ and R² are, each independently, H, a C₁-C₆ alkyl, a C₆-C₁₈ aryl, or a (5-20) atom heteroaryl. For example, R¹ and R² are, each independently, H or C₆-C₁₂ aryl.

R²¹, R²², R²³, and R²⁴ are, each independently, H, or a C₁-C₃ alkyl. For example, R²¹, R²², R²³, and R²⁴ are each H.

F₁ and F₂ are, each independently, CR′ or N, wherein R′ is H, a C₁-C₆ alkyl, a C₆-C₁₈ aryl, or —(Ar₅)_{q}-G. For example, F₁ and F₂ each is a CR′. In another example embodiment, F₁ is C—H and F₂ is a C-G.

Ar₄ or Ar₅ are, each independently, phenyl optionally substituted with one to four C₁-C₃ alkyls. For example Ar₄ or Ar₅, each independently, a moiety represented by the following structural formula:

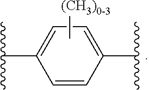

In another example Ar₄ is a moiety represented by the following structural formula:

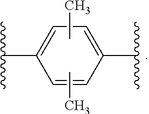

p is 0, 1, or 2. For example, p is 1.

q is 0 or 1. For example, q is 0.

G is H, or a moiety represented by one of the following structural formula:

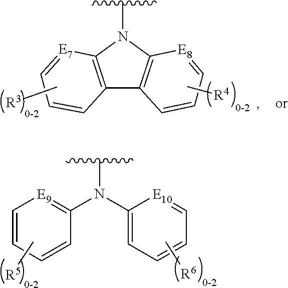

wherein E₇, E₈, E₉, and E₁₀ are, each independently, CH or N, and R³, R⁴, R⁵, and R⁶ are, each independently, a C₁-C₃ alkyl, a C₆-C₁₈ aryl, a halo, or —CN. For example, G is H or a moiety represented by the following structural formula:

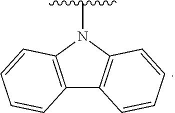 [SMILES: *n1c2ccccc2c2ccccc21]

In structural formula (XII) of the present invention, when E₁, E₂, and E₃ are each N, and F₁ and F₂ are each CR′, then each R′ is not the moiety represented by the structural formula:

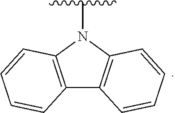 [SMILES: *n1c2ccccc2c2ccccc21]

In an example embodiment of the eighteenth aspect of the present invention, R₁ and R₂ are, each independently, H or C₆-C₁₂ aryl and p is 1, and wherein the values and example values of the remaining variables are described above with respect to structural formula (XII).

In another example embodiment of the eighteenth aspect of the present invention, Ar₄ or Ar₅ are, each independently, a moiety represented by the following structural formula:

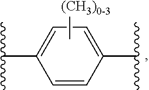

and wherein the values and example values of the remaining variables are described above with respect to structural formula (XII).

In another example embodiment of the eighteenth aspect of the present invention, F₁ and F₂ each is a CR′, and wherein the values and example values of the remaining variables are described above with respect to structural formula (XII).

In another example embodiment of the eighteenth aspect of the present invention, q is 0, and wherein the values and example values of the remaining variables are described above with respect to structural formula (XII).

In another example embodiment of the eighteenth aspect of the present invention, G is H or is a moiety represented by the following structural formula:

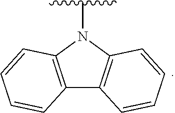 [SMILES: *n1c2ccccc2c2ccccc21]

In another example embodiment of the eighteenth aspect of the present invention, the molecule is represented by the following structural formula:

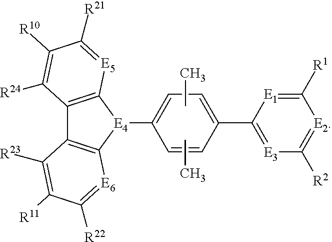

wherein R¹ and R² are, each independently, H or C₆-C₁₂ aryl, and R¹⁰ and R¹¹ are, each independently, H or a moiety represented by the following structural formula:

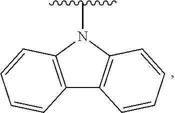 [SMILES: *n1c2ccccc2c2ccccc21]

and wherein the values and example values of the remaining variables are defined above with respect to structural formula (XII).

In another example embodiment of the eighteenth aspect of the present invention, the molecule is represented by the following structural formula:

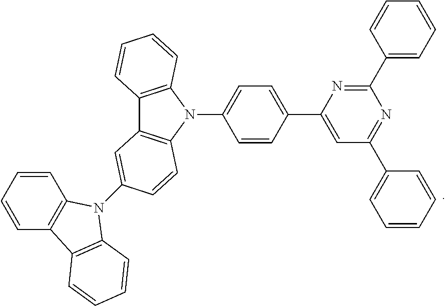 [SMILES: c1ccc(-c2cc(-c3ccc(-n4c5ccccc5c5cc(-n6c7ccccc7c7ccccc76)ccc54)cc3)nc(-c3ccccc3)n2)cc1]

In another example embodiment of the eighteenth aspect of the present invention, the molecule is represented by the following structural formula:

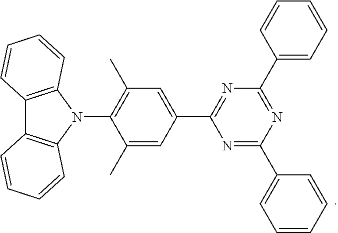 [SMILES: Cc1cc(-c2nc(-c3ccccc3)nc(-c3ccccc3)n2)cc(C)c1-n1c2ccccc2c2ccccc21]

In a nineteenth aspect, the present invention is an organic light-emitting device comprising a first electrode, a second electrode, and an organic layer disposed between the first electrode and the second electrode. In an example embodiment, the organic layer comprises a molecule from any one of the one through eighteen aspects of the present invention described above. In another example embodiment, the organic layer comprises at least one light-emitting molecule represented by a structural formula selected from Tables 1-14. In yet another example embodiment, the organic layer comprises at least one light-emitting molecule represented by any one of the structural formulas in Table 15.

In a twentieth aspect, the present invention is not represented by the structural formulas represented in Table 16.

In a twenty-first aspect, the present invention is not represented by the structural formulas represented in Table 18.

In a twenty-second aspect, the present invention is not represented by the structural formulas represented in Table 16 or Table 18.

In an example embodiment of any one of the one through twenty-second aspects of the present invention described above, the moiety A and the moiety D are different.

In an example embodiment of any one of the one through twenty-second aspects of the present invention described above, the moiety D has a highest occupied molecular orbital (HOMO) energy above −6.5 eV and the moiety A has a lowest unoccupied molecular orbital (LUMO) energy below −0.5 eV.

In an example embodiment of any one of the one through twenty-second aspects of the present invention described above, the molecule is group symmetric or synthetic symmetric.

In an example embodiment of any one of the one through twenty-second aspects of the present invention described above, the molecule is represented by one of the following structural formulas:

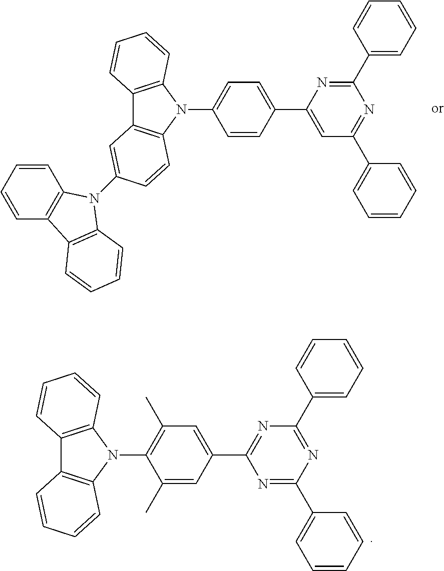 [SMILES: c1ccc(-c2cc(-c3ccc(-n4c5ccccc5c5cc(-n6c7ccccc7c7ccccc76)ccc54)cc3)nc(-c3ccccc3)n2)cc1 ; "or" ; Cc1cc(-c2nc(-c3ccccc3)nc(-c3ccccc3)n2)cc(C)c1-n1c2ccccc2c2ccccc21]

### Combinatorial Assembly and Screening

Example molecules of the present invention having desirable properties, such as color of visible emission, can be constructed from the acceptor, donor, and bridge moieties described above using a combinatorial process described below. While only a few example compounds are illustrated below, it is understood that different combinations of different moieties can be used to create a combinatorial library of compounds. The example moieties below are intended only to illustrate the concepts herein, and are not intended to be limiting.

In the first step, a library of chemical moieties are screened for their abilities to function as acceptor or donor moieties. Example properties examined include desirable quantum mechanical computations such as the ionization potential of the highest occupied molecular orbital (i.e., a “donor” moiety) and the electron affinity of the lowest unoccupied molecular orbital (i.e., an “acceptor” moiety). In an example embodiment, a donor moiety can be selected if it is calculated that it has an ionization potential of greater than or equal to −6.5 eV. In another example embodiment, an acceptor moiety can be selected if it is calculated that it has an electron affinity of less than or equal to −0.5 eV. An example donor moiety selected after screening could be:

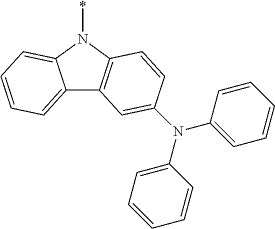 [SMILES: *n1c2ccccc2c2cc(N(c3ccccc3)c3ccccc3)ccc21]

and an example acceptor moiety selected after screening could be:

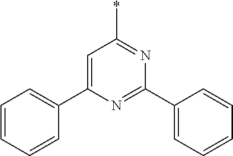 [SMILES: *c1cc(-c2ccccc2)nc(-c2ccccc2)n1]

wherein (*) represents a point of attachment for the donor and acceptor moieties either to each other or to a bridge moiety.

In a second, optional, step, if the selected donor and/or acceptor is “multi-site,” the multi-site donor moiety is combined with a single-site bridge moiety, and/or the multi-site acceptor moiety is combined with a single-site bridge moiety. If the donor and/or acceptor moieties are “single-site” moieties, then multi-site bridge moieties can be combined with the selected moieties. For the purposes of the combinatorial assembly, the number of “sites” refers to how many potentially different moieties can be attached. For example, the moiety below has one “site”:

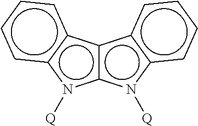

because all moieties attached at the position labeled Q must be the same. Similarly, the moiety below has two “sites” because Q and M can be the same or different:

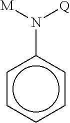

Thus, the nitrogen atom in the molecule is “multi-site.”

In the example moieties from the first step, both moieties are single-site. An example “multi-site” bridge could be:

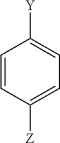

wherein the moieties attached at Y and Z are different. If the donor moiety combines with a bridge, and the acceptor combines with a bridge, the following moieties are created:

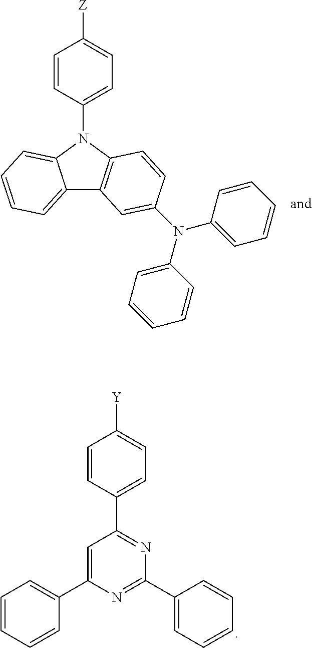

In a third step, the second step can be repeated to continuously add bridge moieties to the molecule. The only limitation is the size of final molecules that are going to be generated. The bridge molecules can be added at position Y or Z, indicated above, and can be the same bridge moiety, or a different bridge moiety. In one example embodiment, the number of bridge moieties can be limited to a number between 0 and 3. In another example, the number of donor moieties and acceptor moieties, or the total molecular weight of the molecule can be limited. In an example embodiment, the molecules are symmetrical. The symmetry can be used to limit the molecules in the combinatorial process to those that are stable. Therefore, for example, an additional bridge moiety added to the moieties from step two could be:

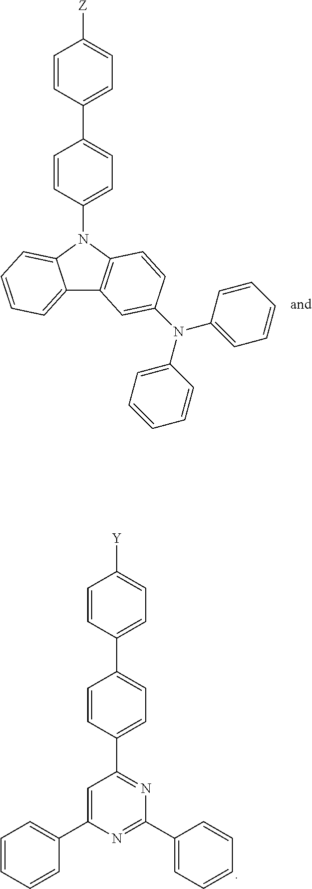

In a fourth step, the unattached point on the bridge moieties only combine with either (1) a donor moiety or an acceptor moiety that does not have a bridge moiety attached; or (2) other bridge moieties that is attached to either an acceptor moiety or a donor moiety such that the size limitation in step three is not violated, and that each molecule comprises at least one donor moiety and one acceptor moiety.

Using the example moieties and the rules described above, the following example molecules can be created:

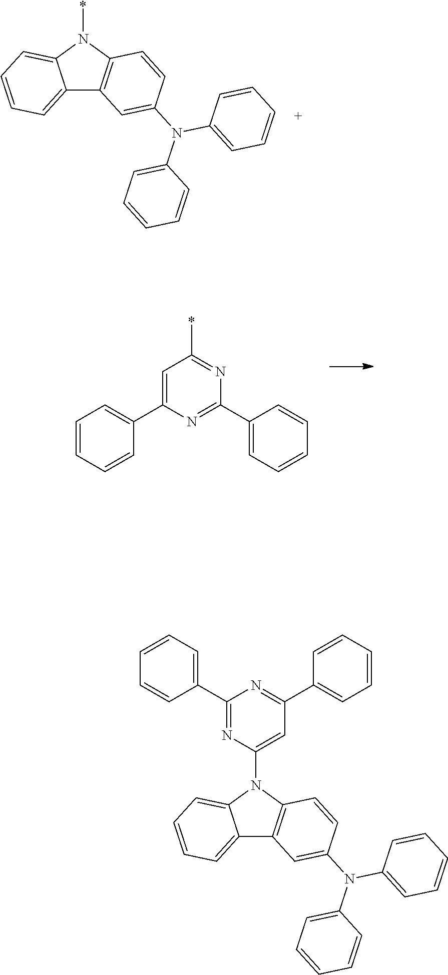 [SMILES: *n1c2ccccc2c2cc(N(c3ccccc3)c3ccccc3)ccc21 ; *c1cc(-c2ccccc2)nc(-c2ccccc2)n1 ; c1ccc(-c2cc(-n3c4ccccc4c4cc(N(c5ccccc5)c5ccccc5)ccc43)nc(-c3ccccc3)n2)cc1]

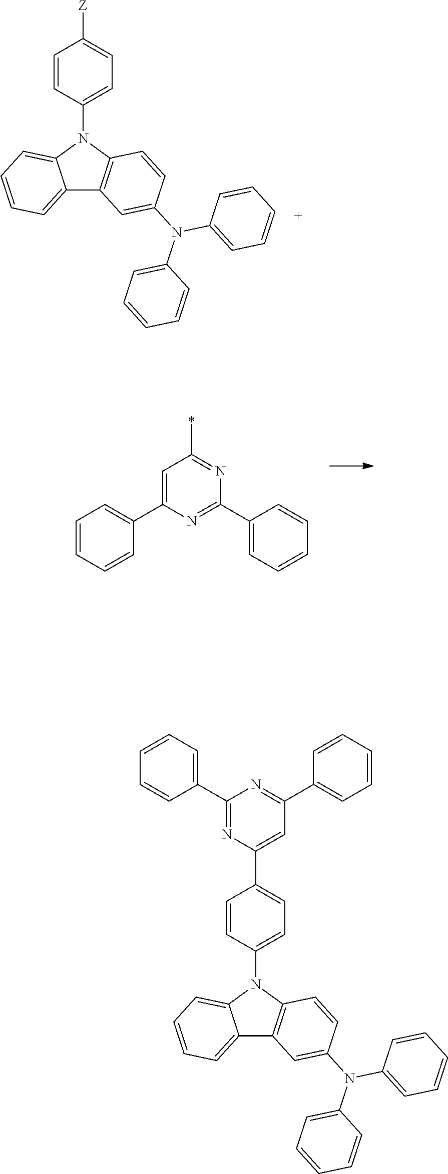

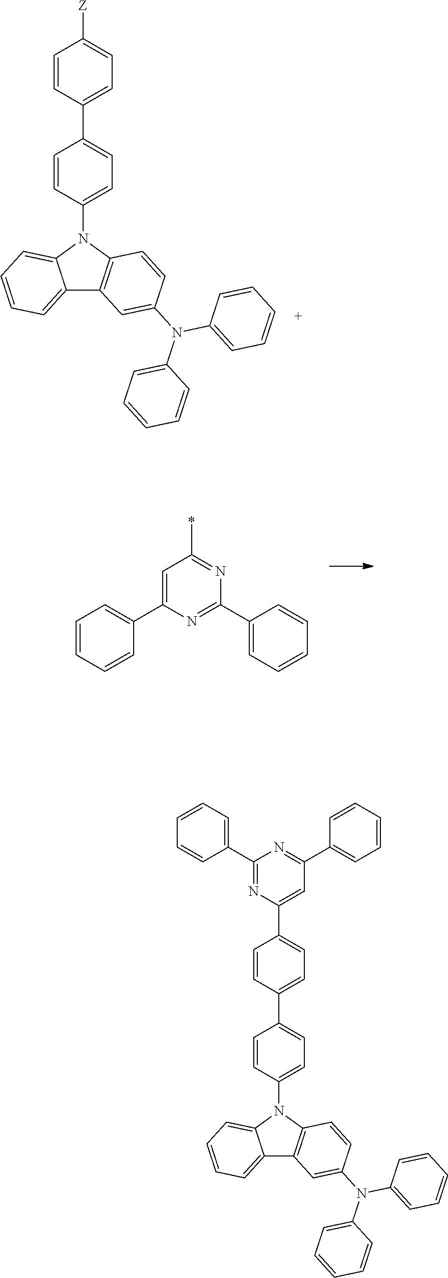

In the fifth step, the combined potential donors, acceptors, and bridges can be screened based on quantum mechanical computations such as desired HOMO and LUMO values, as well as vertical absorption (the energy required to excite the molecule from the ground state to the excited state), rate of decay (S1 to S0 oscillator strength, e.g., how fast and/or how bright the molecule's emission after excitation), estimated color of visible light emission in nanometers, and the singlet-triplet gap (the energy difference between the lowest singlet excited state, S1, the lowest triplet excited state, T1). Examples of these calculations for molecules embodied in the present invention are provided in Table 17.

### Exemplification

### Compound J78

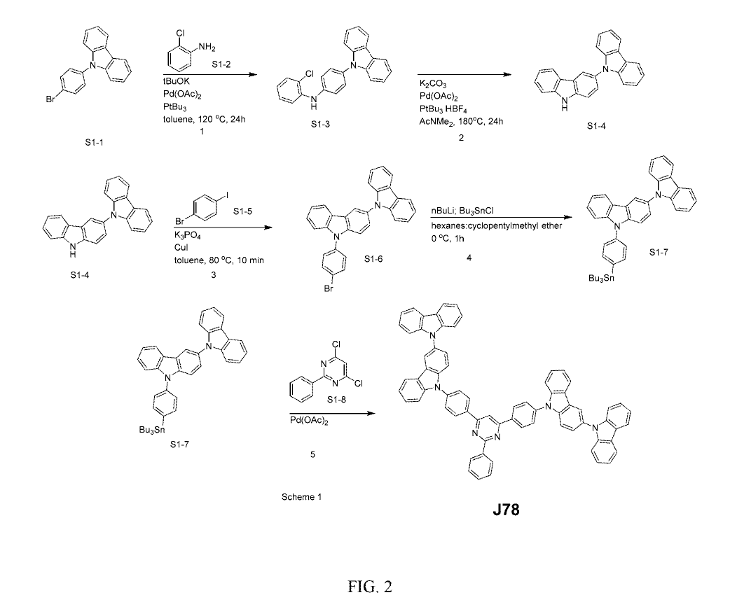

Compound J78 can be synthesized by a person of ordinary skill following Scheme 1 illustrated in FIG. 2. The starting material S1-1 is available for purchase from Alfa Aesar (CAS No. 57102-42-8). The starting material S1-2 is available for purchase from Acros Organics (CAS No. 95-51-2). In the first step compound S1-1 is combined with compound S1-2, potassium tert-butoxide, Pd(OAc)₂, and PtBu₃ in toluene at 120° C. for 24 hours to form compound S1-3. In the second step, compound S1-3 is combined with potassium carbonate, Pd(OAc)₂, and PtBu₃HBF₄, in DMA at 180° C. for 24 hours to form compound S1-4. In the third step, compound S1-4 is combined with compound S1-5 (available for purchase from Acros Organics, CAS No. 589-87-7), K₃PO₄ and copper iodide in toluene at 80° C. for 10 minutes to form compound S1-6. In the fourth step, compound S1-6 is cooled to 0° C. in a hexanes:cyclopentylmethyl ether solution before dropwise addition of nBuLi and subsequent dropwise addition of Bu₃SnCl to form compound S1-7. Compound S1-7 is combined with compound S1-8 (available for purchase from Tokyo Chemical Industry Co., CAS No. 3740-92-9) with Pd(OAc)₂ to form compound J78. It is understood that steps 1, 2, 3, 4, and 5 can be performed and optimized by a person having ordinary skill in the art without undue experimentation.

### Compound K109

Compound K109 can be synthesized by a person of ordinary skill following Scheme 2 illustrated in FIG. 3. In the first step, compound S2-1 (available for purchase from Acros Organics, CAS No. 86-74-8) is combined with compound S2-2 (available for purchase from Alfa Aesar, CAS No. 116632-39-4), K₂CO₃, and CuI in toluene at 80° C. to form compound S2-3. In the second step, compound S2-3 is added to hexanes and cooled to 0° C. before dropwise addition of nBuLi, followed by addition of B(OMe)₃. The reaction can be allowed to stir before being quenched with aqueous HCl to form compound S2-4. In the third step, compound S2-4 is combined with compound S2-5 (available for purchase from Alfa Aesar, CAS No. 3842-55-5), Pd(OAc)₂ and K₃PO₄ in THF at 45° C. for 24 hours to form compound K109. It is understood that steps 1, 2, and 3 can be performed and optimized by a person having ordinary skill in the art without undue experimentation.

### Compound F57

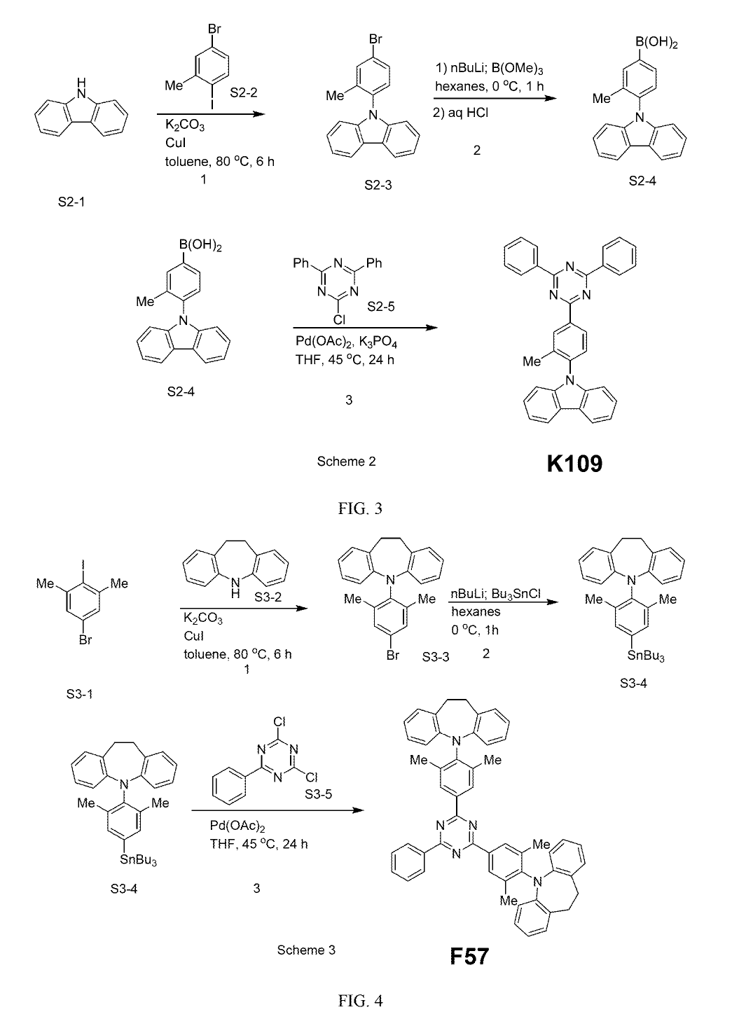

Compound F57 can be synthesized by a person of ordinary skill following Scheme 3 illustrated in FIG. 4. In the first step, compound S3-1 (available for purchase from Combi-Blocks, Inc., CAS No. 206559-43-5) is combined with compound S3-2 (available for purchase from Acros Organics, CAS No. 494-19-9), K₂CO₃ and CuI in toluene at 80° C. to form compound S3-3. In the second step compound S3-3 is cooled to 0° C. in a hexanes:cyclopentylmethyl ether solution before dropwise addition of nBuLi and subsequent dropwise addition of Bu₃SnCl to form compound S3-4. In the third step, compound S3-4 is combined with compound S3-5 (available for purchase from Matrix Scientific, CAS No. 1700-02-3), Pd(OAc)₂ and K₃PO₄ in THF at 45° C. for 24 hours to form compound F57. It is understood that steps 1, 2, and 3 can be performed and optimized by a person having ordinary skill in the art without undue experimentation.

### Compound G32

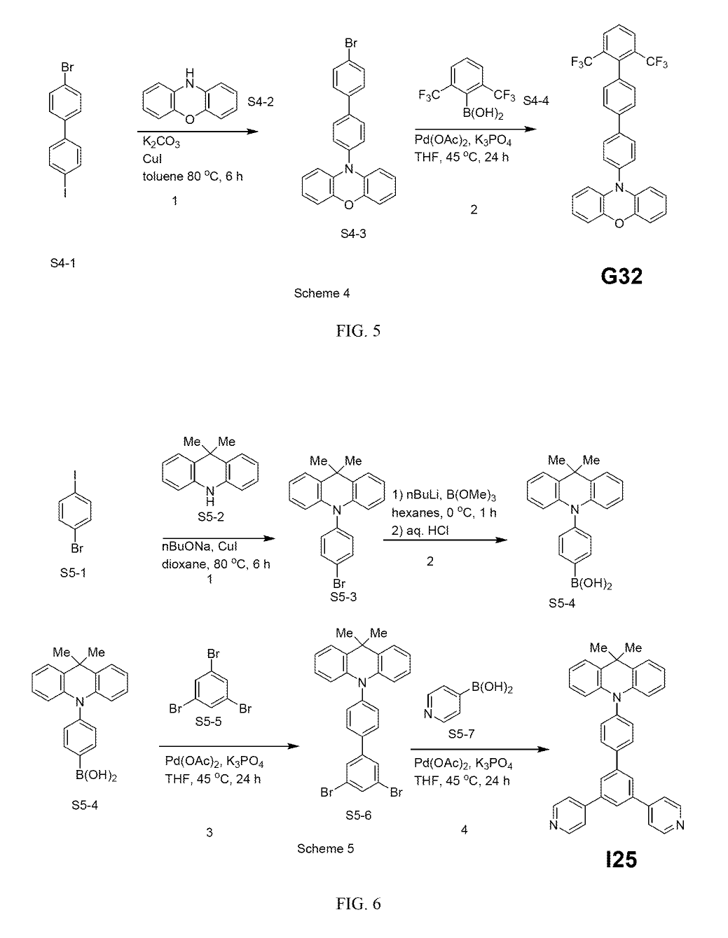

Compound G32 can be synthesized by a person of ordinary skill following Scheme 4 illustrated in FIG. 5. In the first step, compound S4-1 (available for purchase from Alfa Aesar, CAS No. 105946-82-5) is combined with compound S4-2 (available for purchase from Acros Organics, CAS No. 135-67-1), K₂CO₃ and CuI in toluene at 80° C. to form compound S4-3. In the second step, compound S4-3 is combined with compound S4-4 (available for purchase from Alfa Aesar, CAS No. 681812-07-7), Pd(OAc)₂ and K₃PO₄ in THF at 45° C. for 24 hours to form compound G32. It is understood that steps 1 and 2 can be performed and optimized by a person having ordinary skill in the art without undue experimentation.

### Compound 125

Compound 125 can be synthesized by a person of ordinary skill following Scheme 5 illustrated in FIG. 6. In the first step, compound S5-1 (available for purchase from Acros Organics, CAS No. 589-87-7) is combined with compound S5-2 (available for purchase from ArkPharm, Inc., CAS No. 6267-02-3), nBuONa and CuI in dioxane at 80° C. for 6 hours to form compound S5-3. In the second step, compound S5-3 is is added to hexanes and cooled to 0° C. before dropwise addition of nBuLi, followed by addition of B(OMe)₃. The reaction can be allowed to stir before being quenched with aqueous HCl to form compound S5-4. In the third step, compound 55-4 can be combined with compound 55-5 (available for purchase from Acros Organics, CAS No 626-39-1), Pd(OAc)₂ and K₃PO₄ in THF at 45° C. for 24 hours to form compound 55-6. In the fourth step, compound 55-6 can be combined with compound 55-7 (available for purchase from Acros Organics, CAS No 1692-15-5), Pd(OAc)₂ and K₃PO₄ in THF at 45° C. for 24 hours to form compound 125. It is understood that steps 1, 2, 3, and 4 can be performed and optimized by a person having ordinary skill in the art without undue experimentation.

### Compound L23

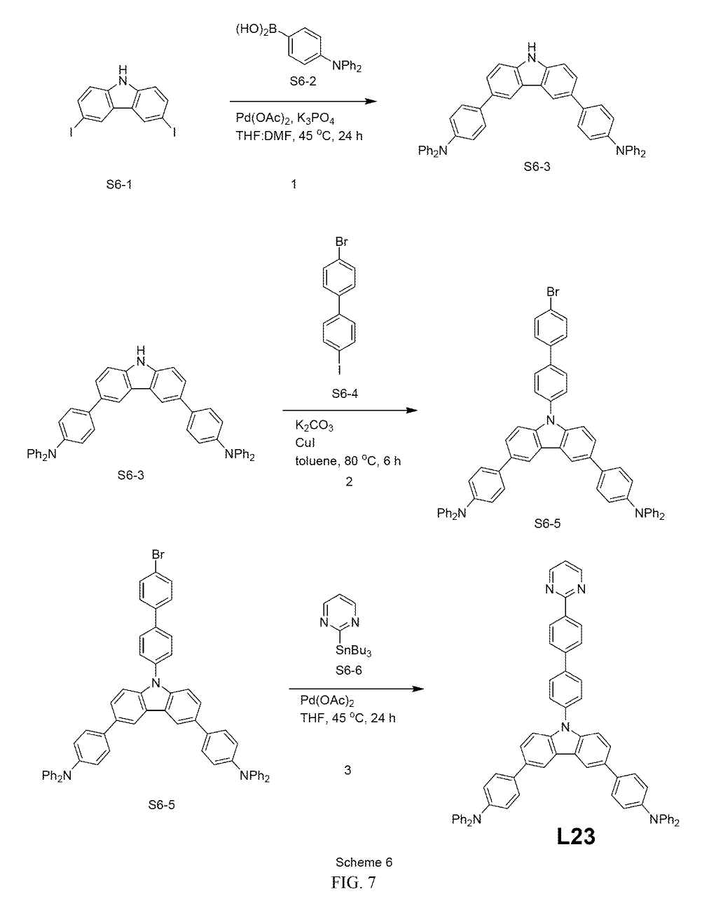

Compound L23 can be synthesized by a person of ordinary skill following Scheme 6 illustrated in FIG. 7. In the first step, compound S6-1 (available for purchase from ArkPharm, Inc., CAS No. 57103-02-3) is combined with compound S6-2 (available for purchase from Alfa Aesar, CAS No. 201802-67-7), Pd(OAc)₂ and K₃PO₄ in THF:DMF at 45° C. and stirred for 24 hours to give compound S6-3. In the second step, compound S6-3 is combined with compound S6-4 (available for purchase from Alfa Aesar, CAS No. 105946-82-5), K₂CO₃ and CuI in toluene at 80° C. and allowed to stir for 6 hours to give compound S6-5. In the third step, compound S6-5 is combined with compound S6-6 (available for purchase from Sigma-Alrdich Co., CAS No. 153435-63-3) and Pd(OAc)₂ in THF at 45° C. and stirred for 24 hours to give compound L23. It is understood that steps 1, 2 and 3 can be performed and optimized by a person having ordinary skill in the art without undue experimentation.

### Compound J70

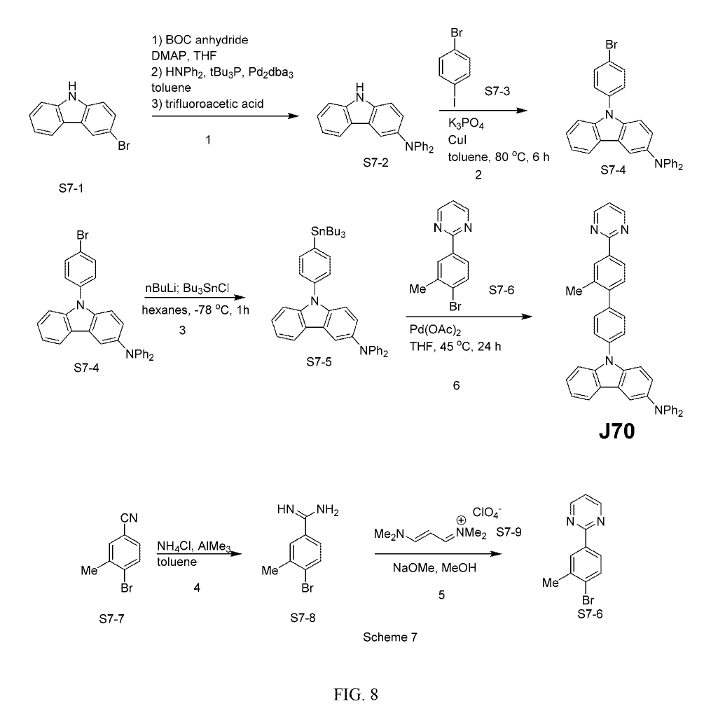

Compound J70 can be synthesized by a person of ordinary skill following Scheme 7 illustrated in FIG. 8. In the first step, compound S7-1 (available for purchase from Acros Organics, CAS No. 1592-95-6) is combined with BOC anhydride and DMAP in THF. The mixture is taken, without purification and combined with HNPh₂, tBu₃P, and Pd₂dba₃ in toluene. This mixture is taken, without purification and combined with TFA to produce compound S7-2. In the second step, compound S7-2 is combined with compound S7-3 (available for purchase from Acros Organics, CAS No. 589-87-7), K₃PO₄ and CuI in toluene at 80° C. and stirred for 6 hours to form compound S7-4. In the third step, compound S7-4 is cooled to −78° C. in hexanes solution before dropwise addition of nBuLi and subsequent dropwise addition of Bu₃SnCl to form compound S7-5.

In the fourth step, compound S7-7 (available for purchase from Sigma-Aldrich, Co. CAS No. 41963-20-6) is combined with ammonium chloride and AlMe₃ in toluene to give compound S7-8. In the fifth step, compound S7-8 is combined with compound S7-9 (compound S7-9 is prepared according to the method described in WO 1998004260) and NaOMe in methanol to form compound S7-6. In the sixth step, compound S7-6 is combined with compound S7-5 and Pd(OAc)₂ in THF at 45° C. and stirred for 24 hours to give compound J70. It is understood that steps 1, 2, 3, 4, 5 and 6 can be performed and optimized by a person having ordinary skill in the art without undue experimentation.

### Compound M22

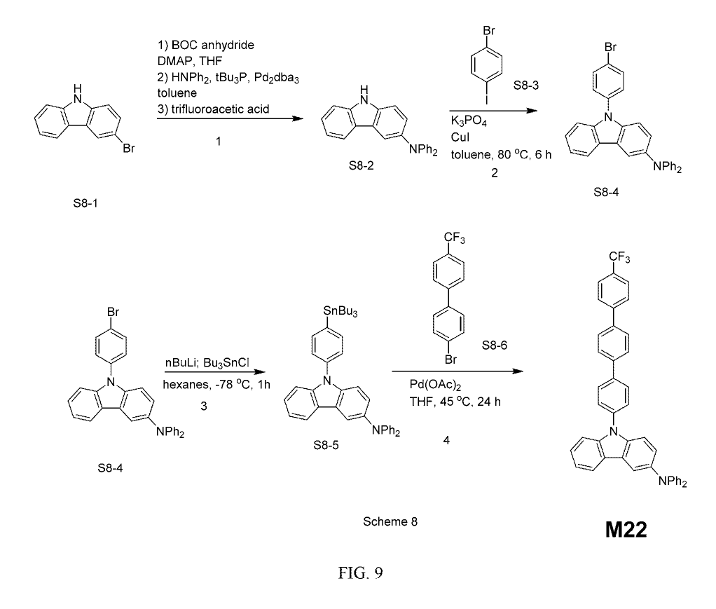

Compound M22 can be synthesized by a person of ordinary skill following Scheme 8 illustrated in FIG. 9. In the first step, compound S8-1 (available for purchase from Acros Organics, CAS No. 1592-95-6) is combined with BOC anhydride and DMAP in THF. The mixture is taken, without purification and combined with HNPh₂, tBu₃P, and Pd₂dba₃ in toluene. This mixture is taken, without purification and combined with TFA to produce compound S8-2. In the second step, compound S8-2 is combined with compound S8-3 (available for purchase from Acros Organics, CAS No. 589-87-7), K₃PO₄ and CuI in toluene at 80° C. and stirred for 6 hours to form compound S8-4. In the third step, compound S8-4 is cooled to −78° C. in hexanes solution before dropwise addition of nBuLi and subsequent dropwise addition of Bu₃SnCl to form compound S8-5. In the fourth step, compound S8-5 is combined with compound S8-6 (available for purchase from A-Tech Chemicals, CAS No. 69231-87-4) and Pd(OAc)₂ in THF at 45° C. and stirred for 24 hours to give compound M22. It is understood that steps 1, 2, 3, and 4 can be performed and optimized by a person having ordinary skill in the art without undue experimentation.

### Compound B5

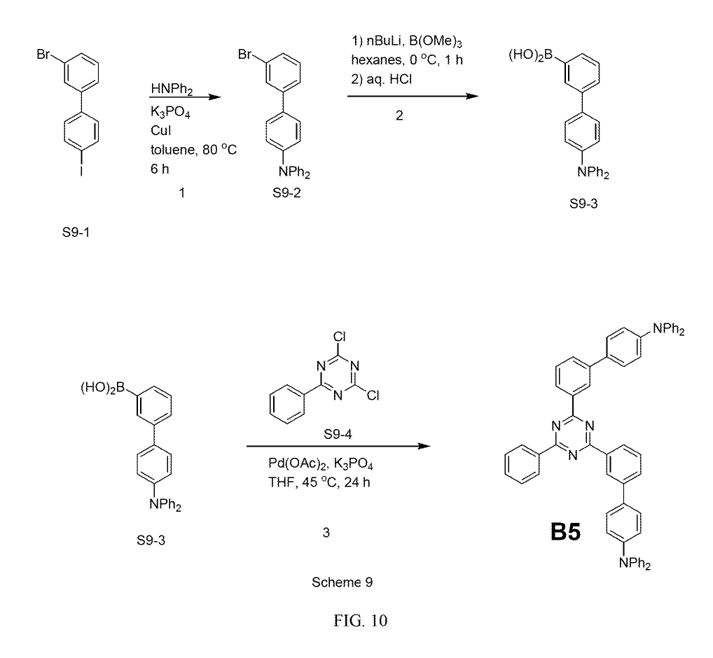

Compound B5 can be synthesized by a person of ordinary skill following Scheme 9 illustrated in FIG. 10. In the first step compound S9-1 (available for purchase from A-Tech Chemicals, CAS No. 187275-73-6) is combined with HNPh₂, K₃PO₄, and CuI in toluene at 80° C. and stirred for 6 hours to form compound S9-2. In the second step, compound S9-2 is added to hexanes and cooled to 0° C. before dropwise addition of nBuLi, followed by addition of B(OMe)₃. The reaction can be allowed to stir before being quenched with aqueous HCl to form compound S9-3. In the third step, compound S9-3 is combined with compound S9-4 (available for purchase from Matrix Scientific, CAS No. 1700-02-3), Pd(OAc)₂ and K₃PO₄ in THF at 45° C. for 24 hours to form compound B5. It is understood that steps 1, 2, and 3 can be performed and optimized by a person having ordinary skill in the art without undue experimentation.

### Compound H52

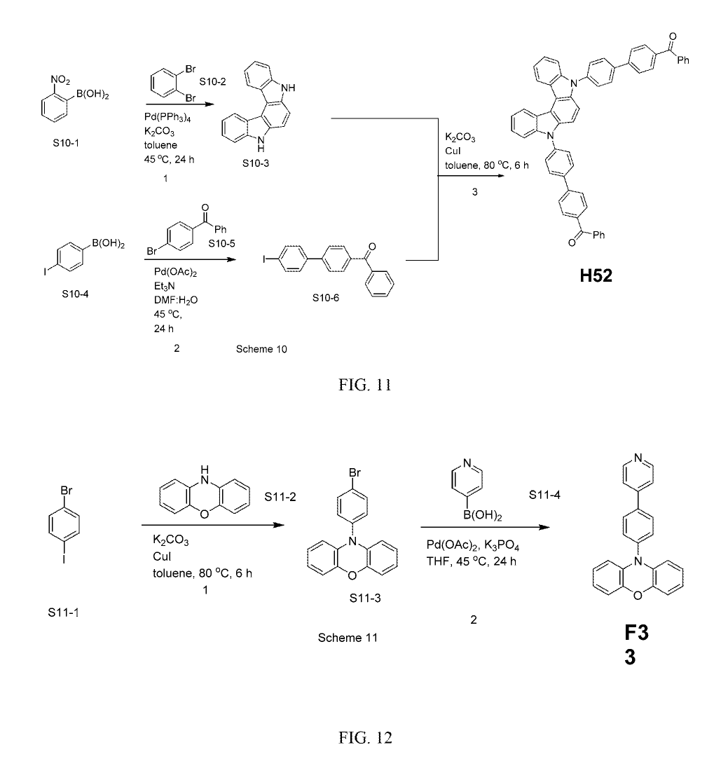

Compound H52 can be synthesized by a person of ordinary skill following Scheme 10 illustrated in FIG. 11. In the first step, compound S10-1 (available for purchase from Acros Organics, CAS No. 5570-19-4) is combined compound S10-2 (available for purchase from Acros Organics, 583-53-9), Pd(PPh₃)₄ and K₂CO₃ in toluene at 45° C. and stirred for 24 hours to form compound S10-3. In the second step, compound S10-4 (available for purchase from Acros Organics CAS No. 5122-99-6) is combined with compound S10-5 (available for purchase from Acros Organics, CAS No. 90-90-4), Pd(OAc)₂ and triethylamine in a solution of DMF:H₂O at 45° C. and stirred for 24 hours to give compound S10-6. In the third step, compound S10-6 and compound S10-3 are combined with K₂CO₃ and CuI in toluene at 80° C. and stirred for 6 hours to give compound H52. It is understood that steps 1, 2, and 3 can be performed and optimized by a person having ordinary skill in the art without undue experimentation.

### Compound F33

Compound F33 can be synthesized by a person of ordinary skill following Scheme 11 illustrated in FIG. 12. In the first step, compound S11-1 (available for purchase from Acros Organics, CAS No. 589-87-7) is combined with compound S11-2 (available for purchase from Acros Organics, CAS No. 135-67-1), K₂CO₃ and CuI in toluene at 80° C. to form compound S11-3. In the second step, compound S11-3 is combined with compound S11-4 (available for purchase from Acros Organics, CAS 1692-15-5), Pd(OAc)₂ and K₃PO₄ in THF at 45° C. for 24 hours to form compound F33. It is understood that steps 1 and 2 can be performed and optimized by a person having ordinary skill in the art without undue experimentation.

### Compound E3

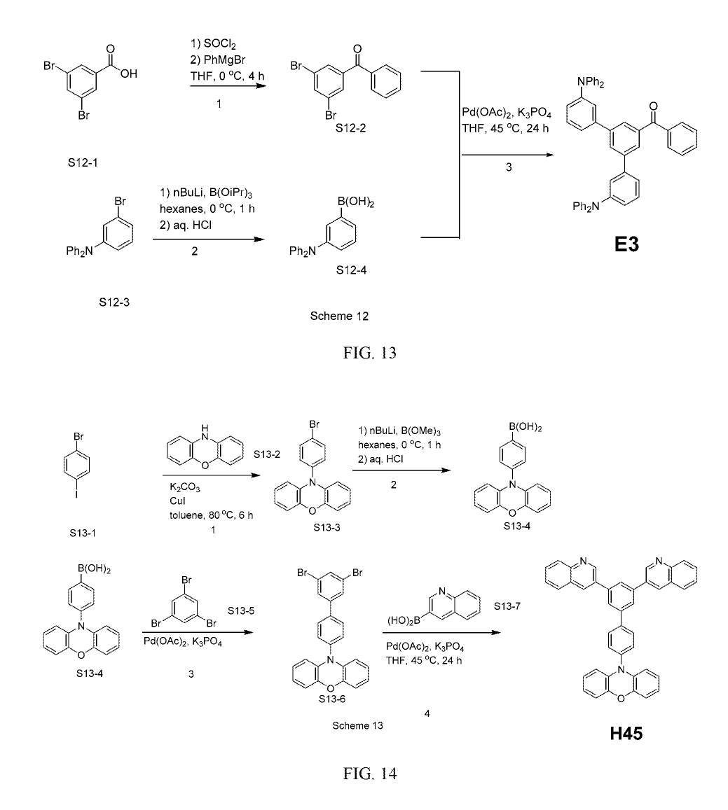

Compound E3 can be synthesized by a person of ordinary skill following Scheme 12 illustrated in FIG. 13. In the first step compound S12-1 is combined with SOCl₂. The intermediate is taken without purification and combined with PhMgBr in THF at 0° C. and stirred for 4 hours to give compound S12-2. In the second step, compound S12-3 (available for purchase from Sigma-Aldrich Co., CAS No. 78600-33-6) is added to hexanes and cooled to 0° C. before dropwise addition of nBuLi. B(OiPr)₃ is subsequently added and the reaction is allowed to stir for 1 hour before being quenched with aqueous HCl to give compound S12-4. In the third step, compound S12-4 and compound S12-3 are combined with Pd(OAc)₂ and K₃PO₄ in THF at 45° C. and stirred for 24 hours to give compound E3. It is understood that steps 1, 2, and 3 can be performed and optimized by a person having ordinary skill in the art without undue experimentation.

### Compound H45

Compound H45 can be synthesized by a person of ordinary skill following Scheme 13 illustrated in FIG. 14. In the first step, compound S13-1 (available for purchase from Acros Organics, CAS No. 589-87-7) is combined with compound 513-2 (available for purchase from Acros Organics, CAS No. 135-67-14), K₂CO₃ and CuI in toluene at 80° C. for 6 hours to form compound S13-3. In the second step, compound S13-3 is is added to hexanes and cooled to 0° C. before dropwise addition of nBuLi, followed by addition of B(OMe)₃. The reaction can be allowed to stir before being quenched with aqueous HCl to form compound S13-4. In the third step, compound S13-4 can be combined with compound S13-5 (available for purchase from Acros Organics, CAS No 626-39-1), Pd(OAc)₂ and K₃PO₄ in THF at 45° C. for 24 hours to form compound S13-6. In the fourth step, compound S13-6 can be combined with compound S13-7 (available for purchase from Acros Organics, CAS No 191162-39-7), Pd(OAc)₂ and K₃PO₄ in THF at 45° C. for 24 hours to form compound H45. It is understood that steps 1, 2, 3, and 4 can be performed and optimized by a person having ordinary skill in the art without undue experimentation.

### Compound J62

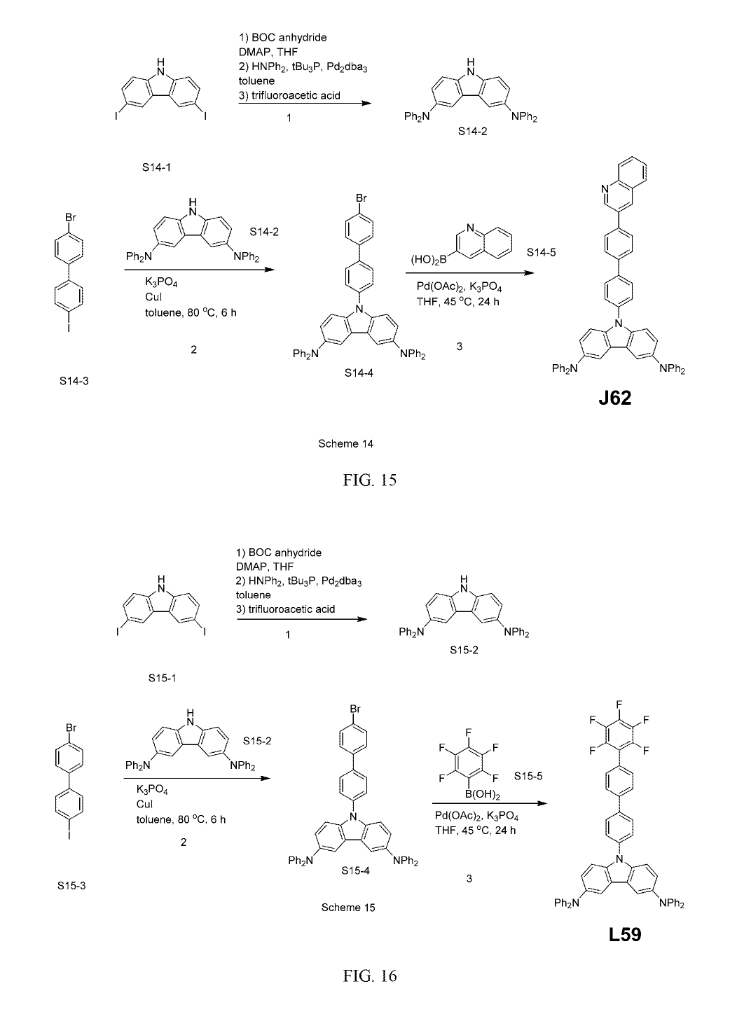

Compound J62 can be synthesized by a person of ordinary skill following Scheme 14 illustrated in FIG. 15. In the first step, compound S14-1 (available for purchase from ArkPharm, Inc., CAS No. 57103-02-3) is combined with BOC anhydride and DMAP in THF. The mixture is taken, without purification and combined with HNPh₂, tBu₃P, and Pd₂dba₃ in toluene. This mixture is taken, without purification and combined with TFA to produce compound S14-2. In the second step, compound S14-2 is combined with compound S14-3 (available for purchase from Alfa Aesar, CAS No. 105946-82-5), K₃PO₄ and CuI in toluene at 80° C. and stirred for 6 hours to form compound S14-4. In the third step, compound S14-4 is combined with compound S14-5 (available for purchase from Acros Organics, CAS No. 191162-39-7), Pd(OAc)₂, and K₂CO₃ in THF at 45° C. and stirred for 24 hours to give compound J62. It is understood that steps 1, 2, and 3 can be performed and optimized by a person having ordinary skill in the art without undue experimentation.

### Compound L59

Compound L59 can be synthesized by a person of ordinary skill following Scheme 15 illustrated in FIG. 16. In the first step, compound S15-1 (available for purchase from ArkPharm, Inc., CAS No. 57103-02-3) is combined with BOC anhydride and DMAP in THF. The mixture is taken, without purification and combined with HNPh₂, tBu₃P, and Pd₂dba₃ in toluene. This mixture is taken, without purification and combined with TFA to produce compound S15-2. In the second step, compound S15-2 is combined with compound S15-3 (available for purchase from Alfa Aesar, CAS No. 105946-82-5), K₃PO₄ and CuI in toluene at 80° C. and stirred for 6 hours to form compound S15-4. In the third step, compound S15-4 is combined with compound S15-5 (available for purchase from Alfa Aesar, CAS No. 1582-24-7), Pd(OAc)₂, and K₂CO₃ in THF at 45° C. and stirred for 24 hours to give compound L59. It is understood that steps 1, 2, and 3 can be performed and optimized by a person having ordinary skill in the art without undue experimentation.

### Compound 199

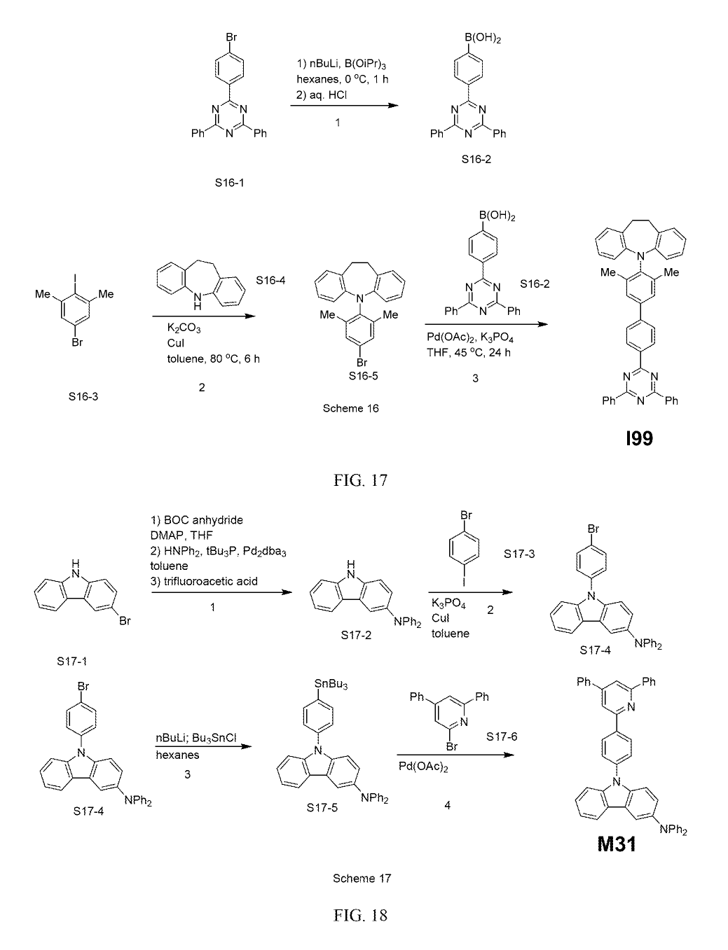

Compound 199 can be synthesized by a person of ordinary skill following Scheme 16 illustrated in FIG. 17. In the first step, compound S16-1 (available for purchase from ArkPharm, Inc., CAS No. 23449-08-3) is added to hexanes and cooled to 0° C. before dropwise addition of nBuLi. B(OiPr)₃ is subsequently added and the reaction is allowed to stir for 1 hour before being quenched with aqueous HCl to give compound S16-2. In the second step, compound 16-3 (available for purchase from Combi-Blocs, Inc., CAS No. 206559-43-5) is combined with compound S16-4 (available for purchase from Acros Organics, CAS No. 494-19-9), K₂CO₃, and CuI in toluene at 80° C. and stirred for 24 hours to give compound S16-5. In the third step, compound 16-5 is combined with compound 16-5, Pd(OAc)₂ and K₃PO₄ in THF at 45° C. and stirred for 24 hours to give compound 199.

### Compound M31

Compound M31 can be synthesized by a person of ordinary skill following Scheme 17 illustrated in FIG. 18. In the first step, compound S17-1 (available for purchase from Acros Organics, CAS No. 1592-95-6) is combined with BOC anhydride and DMAP in THF. The mixture is taken, without purification and combined with HNPh₂, tBu₃P, and Pd₂dba₃ in toluene. This mixture is taken, without purification and combined with TFA to produce compound S17-2. In the second step, compound S17-2 is combined with compound S17-3 (available for purchase from Acros Organics, CAS No. 589-87-7), K₃PO₄ and CuI in toluene at 80° C. and stirred for 6 hours to form compound S17-4. In the third step, compound S17-4 is cooled to −78° C. in hexanes solution before dropwise addition of nBuLi and subsequent dropwise addition of Bu₃SnCl to form compound S17-5. In the fourth step, compound S17-6 is combined with compound S17-5 and Pd(OAc)₂ in THF at 45° C. and stirred for 24 hours to give compound M31. It is understood that steps 1, 2, 3, and 4 can be performed and optimized by a person having ordinary skill in the art without undue experimentation.

### Compound K28

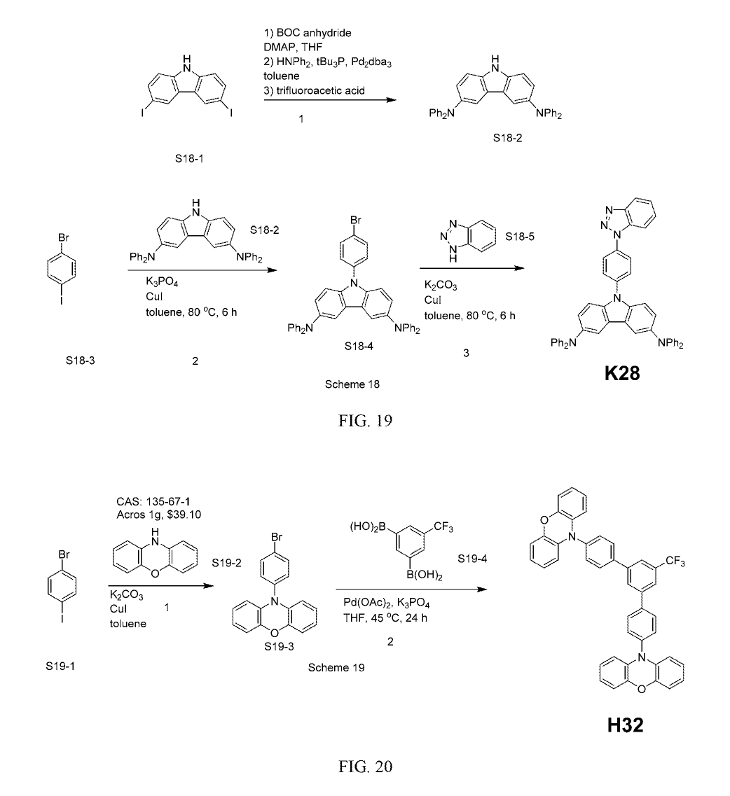

Compound K28 can be synthesized by a person of ordinary skill following Scheme 18 illustrated in FIG. 19. In the first step, compound S18-1 (available for purchase from ArkPharm, Inc., CAS No. 57103-02-3) is combined with BOC anhydride and DMAP in THF. The mixture is taken, without purification and combined with HNPh₂, tBu₃P, and Pd₂dba₃ in toluene. This mixture is taken, without purification and combined with TFA to produce compound S18-2. In the second step, compound S18-2 is combined with compound S18-3 (available for purchase from Acros Organics, CAS No. 589-87-7), K₃PO₄ and CuI in toluene at 80° C. and stirred for 6 hours to form compound S18-4. In the third step, compound S18-4 is combined with compound S14-5 (available for purchase from Acros Organics, CAS No. 95-14-7), K₂CO₃, and CuI in toluene at 80° C. and stirred for 6 hours to give compound K28. It is understood that steps 1, 2, and 3 can be performed and optimized by a person having ordinary skill in the art without undue experimentation.

### Compound H32

Compound H32 can be synthesized by a person of ordinary skill following Scheme 19 illustrated in FIG. 20. In the first step, compound S19-1 (available for purchase from Acros Organics, CAS No. 589-87-7) is combined with compound S19-2 (available for purchase from Acros Organics, CAS No. 135-67-1), K₂CO₃ and CuI in toluene at 80° C. to form compound S19-3. In the second step, compound S19-3 is combined with compound S19-4 (available for purchase from Alfa Aesar, CAS No. 913835-35-5), Pd(OAc)₂ and K₃PO₄ in THF at 45° C. for 24 hours to form compound H32. It is understood that steps 1 and 2 can be performed and optimized by a person having ordinary skill in the art without undue experimentation.

### Compound B231

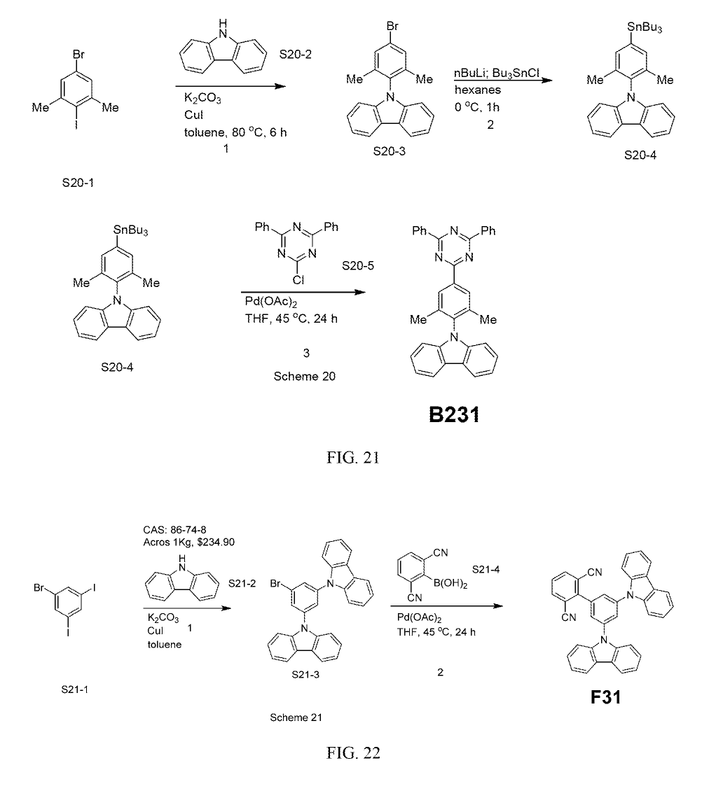

Compound B231 can be synthesized by a person of ordinary skill following Scheme 20 illustrated in FIG. 21. In the first step, compound S20-1 (available for purchase from Combi-Blocks, Inc., CAS No. 206559-43-5) is combined with compound S20-2 (available for purchase from Acros Organics, CAS No. 86-74-8), K₂CO₃ and CuI in toluene at 80° C. to form compound S20-3. In the second step compound S20-3 is cooled to 0° C. in hexanes before dropwise addition of nBuLi and subsequent dropwise addition of Bu₃SnCl to form compound S20-4. In the third step, compound S20-4 is combined with compound S20-5 (available for purchase from Alfa Aesar, CAS No. 3842-55-5), Pd(OAc)₂ and K₃PO₄ in THF at 45° C. for 24 hours to form compound B231. It is understood that steps 1, 2, and 3 can be performed and optimized by a person having ordinary skill in the art without undue experimentation.

### Compound F31

Compound F31 can be synthesized by a person of ordinary skill following Scheme 21 illustrated in FIG. 22. In the first step, compound S21-1 (available for purchase from Spectra Scientific, CAS No. 149428-64-8) is combined with compound S21-2 (available for purchase from Acros Organics, CAS No. 86-74-8), K₂CO₃ and CuI in toluene at 80° C. to form compound S21-3. In the second step, compound S21-3 is combined with compound S21-4 (available for purchase from Arch Bioscience, CAS No. 232275-35-3), Pd(OAc)₂ and K₃PO₄ in THF at 45° C. for 24 hours to form compound F31. It is understood that steps 1 and 2 can be performed and optimized by a person having ordinary skill in the art without undue experimentation.

### Compound 127

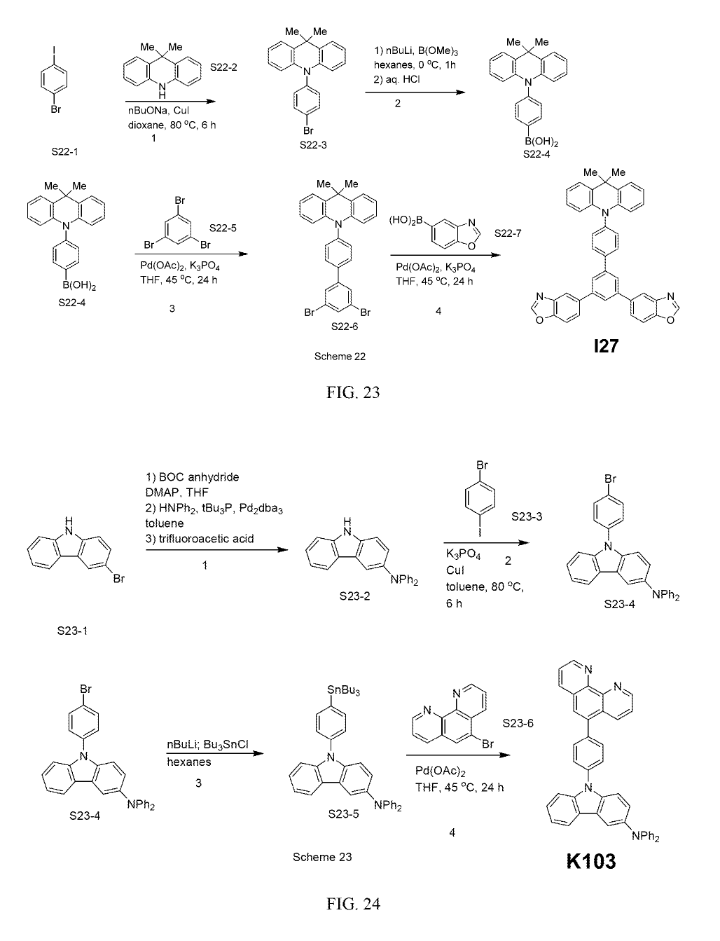

Compound 127 can be synthesized by a person of ordinary skill following Scheme 22 illustrated in FIG. 23. In the first step, compound S22-1 (available for purchase from Acros Organics, CAS No. 589-87-7) is combined with compound S22-2 (available for purchase from ArkPharm, Inc., CAS No. 6267-02-3), nBuONa and CuI in dioxane at 80° C. for 6 hours to form compound S22-3. In the second step, compound S22-3 is is added to hexanes and cooled to 0° C. before dropwise addition of nBuLi, followed by addition of B(OMe)₃. The reaction can be allowed to stir before being quenched with aqueous HCl to form compound S22-4. In the third step, compound S22-4 can be combined with compound S22-5 (available for purchase from Acros Organics, CAS No 626-39-1), Pd(OAc)₂ and K₃PO₄ in THF at 45° C. for 24 hours to form compound S22-6. In the fourth step, compound S22-6 can be combined with compound S22-7 (available for purchase from Anichem, Inc., CAS No. 1443112-43-3), Pd(OAc)₂ and K₃PO₄ in THF at 45° C. for 24 hours to form compound 127. It is understood that steps 1, 2, 3, and 4 can be performed and optimized by a person having ordinary skill in the art without undue experimentation.

### Compound K103

Compound K103 can be synthesized by a person of ordinary skill following Scheme 23 illustrated in FIG. 24. In the first step, compound S23-1 (available for purchase from Acros Organics, CAS No. 1592-95-6) is combined with BOC anhydride and DMAP in THF. The mixture is taken, without purification and combined with HNPh₂, tBu₃P, and Pd₂dba₃ in toluene. This mixture is taken, without purification and combined with TFA to produce compound S23-2. In the second step, compound S23-2 is combined with compound S23-3 (available for purchase from Acros Organics, CAS No. 589-87-7), K₃PO₄ and CuI in toluene at 80° C. and stirred for 6 hours to form compound S23-4. In the third step, compound S23-4 is cooled to −78° C. in hexanes solution before dropwise addition of nBuLi and subsequent dropwise addition of Bu₃SnCl to form compound S23-5. In the fourth step, compound S23-5 is combined with compound S23-6 (available for purchase from eNovation Chemicals, CAS No. 40000-20-2) and Pd(OAc)₂ in THF at 45° C. and stirred for 24 hours to give compound K103. It is understood that steps 1, 2, 3 and 4 can be performed and optimized by a person having ordinary skill in the art without undue experimentation.

### Compound L3

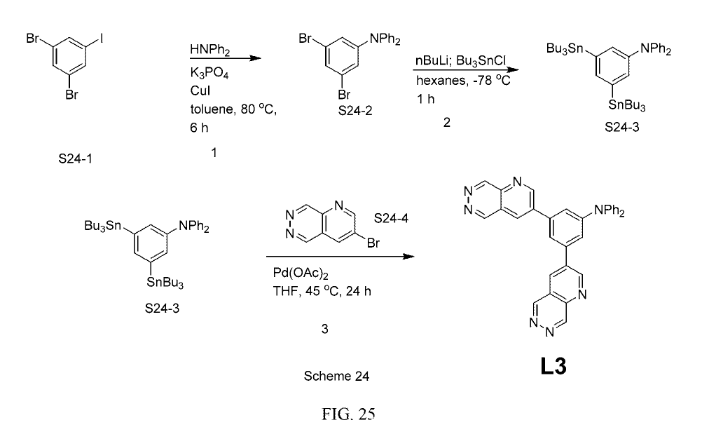

Compound L3 can be synthesized by a person of ordinary skill following Scheme 24 illustrated in FIG. 25. In the first step, compound S24-1 (available for purchase from Combi-Blocs, Inc., CAS No. 19752-57-9) is combined with HNPh₂, K₃PO₄, and CuI in toluene at 80° C. and stirred for 6 hours to form compound S24-2. In the second step compound S24-2 is cooled to −78° C. in hexanes before dropwise addition of nBuLi and subsequent dropwise addition of Bu₃SnCl to form compound S24-3. In the third step, compound 24-3 is combined with compound 24-4 (available for purchase from Aces Pharma, CAS No. 23589-95-9) and Pd (OAc)₂ in THF at 45° C. and allowed to stir for 24 hours to give compound L3. It is understood that steps 1, 2, and 3 can be performed and optimized by a person having ordinary skill in the art without undue experimentation.

### Compound K45

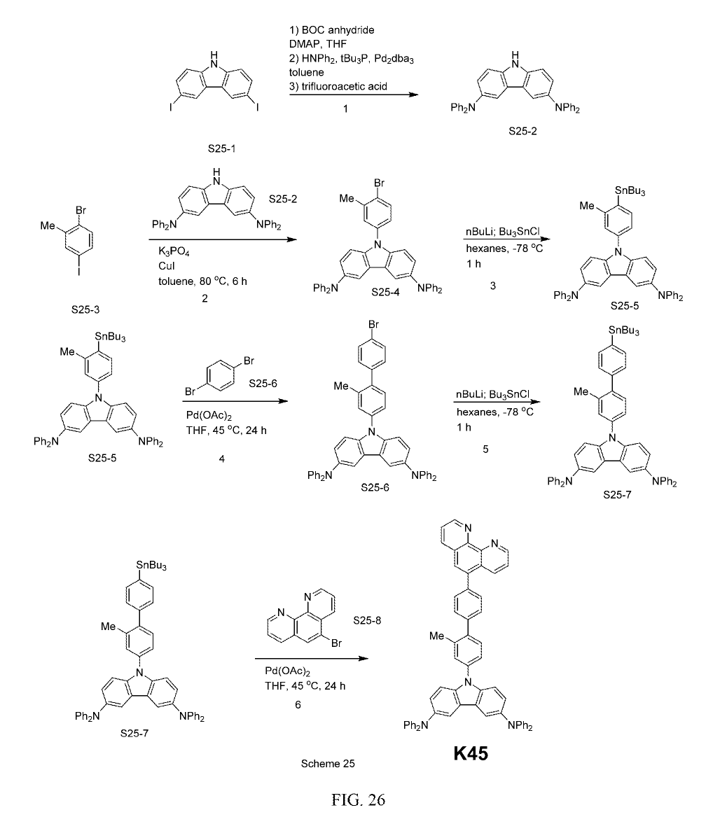

Compound K45 can be synthesized by a person of ordinary skill following Scheme 25 illustrated in FIG. 26. In the first step, compound S25-1 (available for purchase from ArkPharm, Inc., CAS No. 57103-02-3) is combined with BOC anhydride and DMAP in THF. The mixture is taken, without purification and combined with HNPh₂, tBu₃P, and Pd₂dba₃ in toluene. This mixture is taken, without purification and combined with TFA to produce compound S25-2. In the second step, compound S25-2 is combined with compound S25-3 (available for purchase from Alfa Aesar, CAS No. 202865-85-8), K₃PO₄ and CuI in toluene at 80° C. and stirred for 6 hours to form compound S25-4. In the third step, compound S25-4 is cooled to −78° C. in hexanes solution before dropwise addition of nBuLi and subsequent dropwise addition of Bu₃SnCl to form compound S25-5. In the fourth step, compound 25-5 is combined with compound S25-6 (available for purchase from Acros Organics, CAS No. 106-37-6) and Pd(OAc)₂ in THF at 45° C. and stirred for 24 hours to give compound S25-6. In the fifth step, compound S25-6 is cooled to −78° C. in hexanes solution before dropwise addition of nBuLi and subsequent dropwise addition of Bu₃SnCl to form compound S25-7. In the sixth step, compound S25-7 is combined with compounds S25-8 (available for purchase from eNovation Chemicals, CAS No. 40000-20-2) and Pd(OAc)₂ in THF at 45° C. and stirred for 24 hours to give compound K45. It is understood that steps 1, 2, 3, 4, 5, and 6 can be performed and optimized by a person having ordinary skill in the art without undue experimentation.

### Compound M53

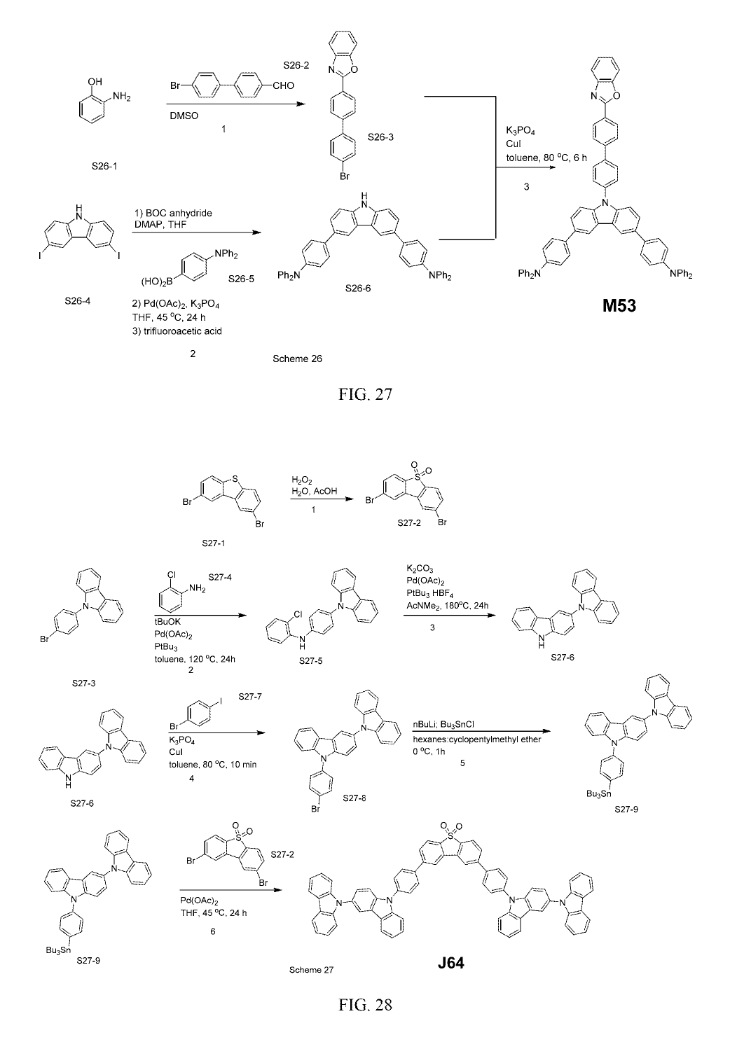

Compound M53 can be synthesized by a person of ordinary skill following Scheme 26 illustrated in FIG. 27. In the first step, compound S26-1 (available for purchase from Acros Organics, CAS No. 95-55-6) is combined with compound S26-2 (available for purchase from Matrix Chemicals, CAS No. 50670-58-1) in DMSO to form compound 26-3. In the second step, compound 26-4 (available for purchase from ArkPharm, CAS No. 57103-02-3) is combined with BOC anhydride and DMAP in THF. The mixture is taken, without purification and combined with compound 26-5 (available for purchase from Sigma-Aldrich Co., CAS No. 201802-67-7). This mixture is taken, without purification and combined with TFA to produce compound S26-6. In the third step, compound 26-6 is combined with compound 26-3, K₃PO₄, and CuI in toluene at 80° C. and stirred for 24 hours to give M53. It is understood that steps 1, 2, and 3 can be performed and optimized by a person having ordinary skill in the art without undue experimentation.

### Compound J64

Compound J64 can be synthesized by a person of ordinary skill following Scheme 27 illustrated in FIG. 28. In the first step, compound S27-1 (available for purchase from ArkPharm, CAS No. 31574-87-5) is stirred with H₂O₂, H₂O, and AcOH to form compound S27-2. In the second step, compound S27-3 (available for purchase from Alfa Aesar, CAS No. 57102-42-8) is combined with compound S27-4 (available for purchase from Acros Organics, CAS No. 95-51-2), tBuOK, Pd(OAc)₂, and PtBu₃ in toluene at 120° C. and stirred for 24 hours to form compound S27-5. In the third step, compound S27-5 is combined with K₂CO₃, Pd(OAc)₂, PtBu₃, and HBF₄ in DMA at 180° C. and stirred for 24 hours to form compound S27-6.

In the fourth step, compound 27-6 is combined with compound 27-7 (available for purchase from Acros Organics, CAS No. 589-87-7), K₃PO₄, and CuI in toluene at 80° C. for 10 minutes to form compound S27-8. In the fifth step, compound S27-8 is cooled to 0° C. in a hexanes:cyclopentylmethyl ether solution before dropwise addition of nBuLi and subsequent dropwise addition of Bu₃SnCl to form compound S27-9. In the sixth step, compound 27-9 is combined with compound S27-2 and Pd(OAc)₂ in THF at 45° C. and stirred for 24 hours to form compound J64.

### Compound S28-8

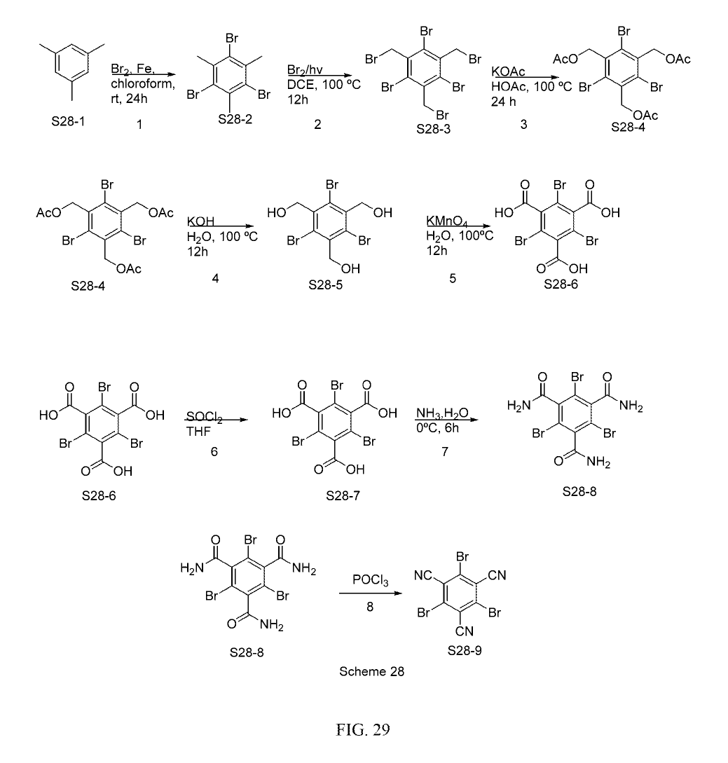

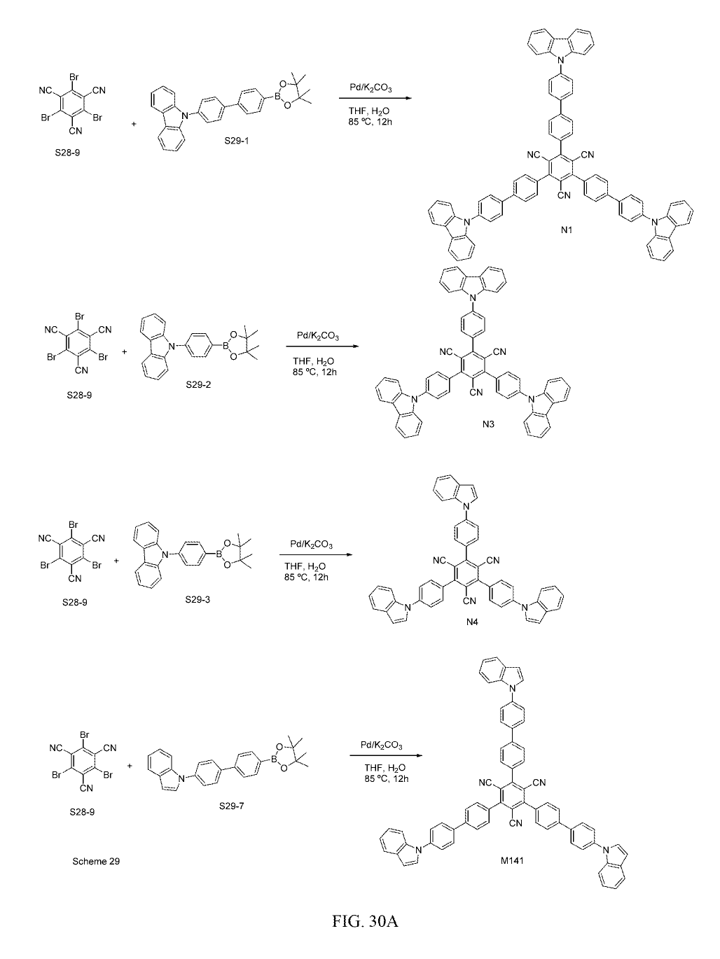

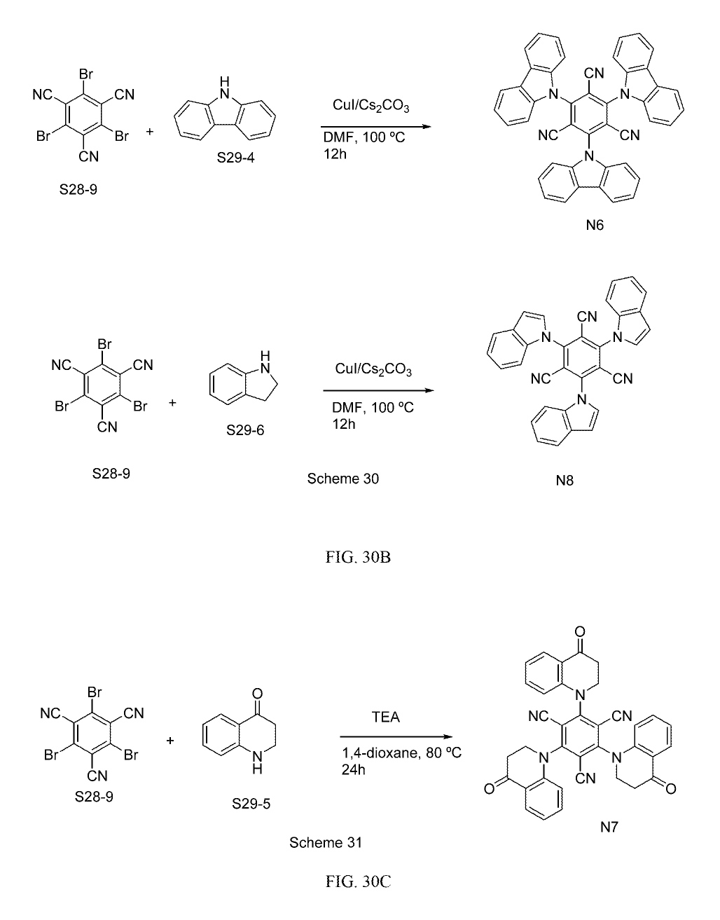

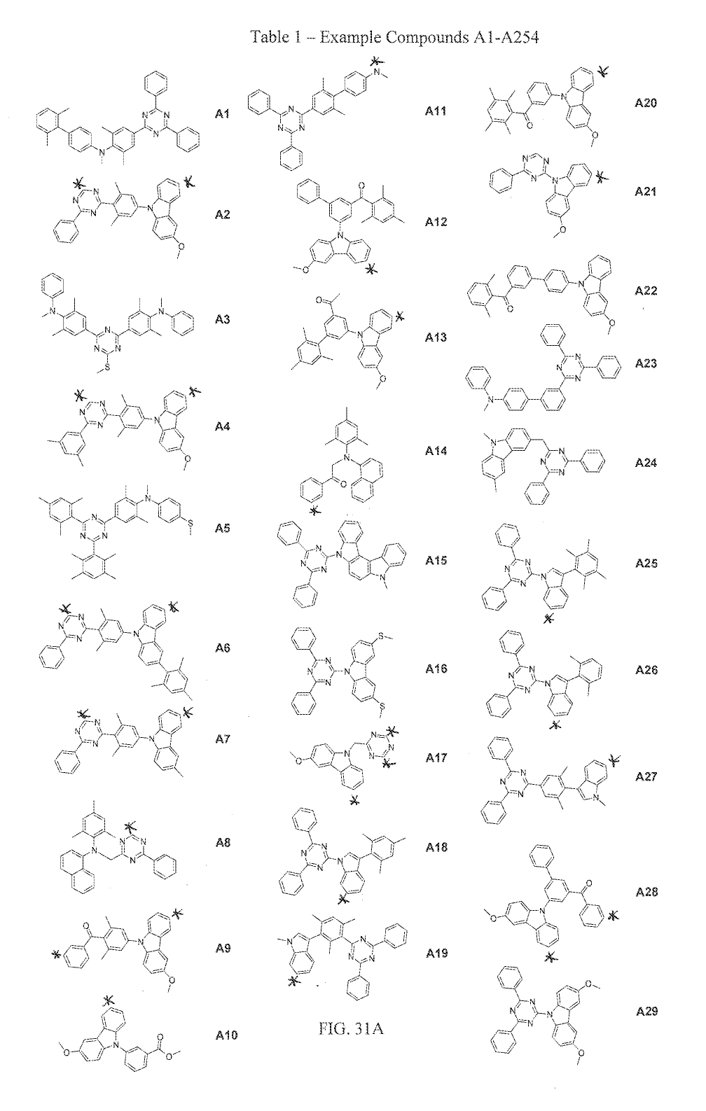

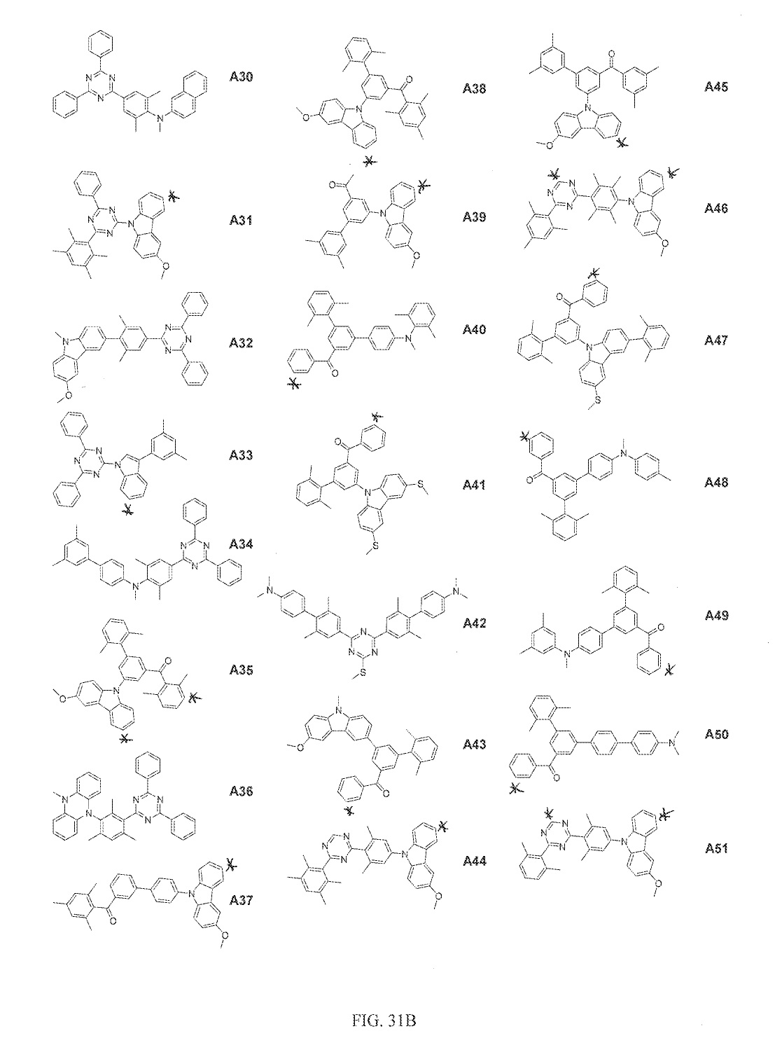

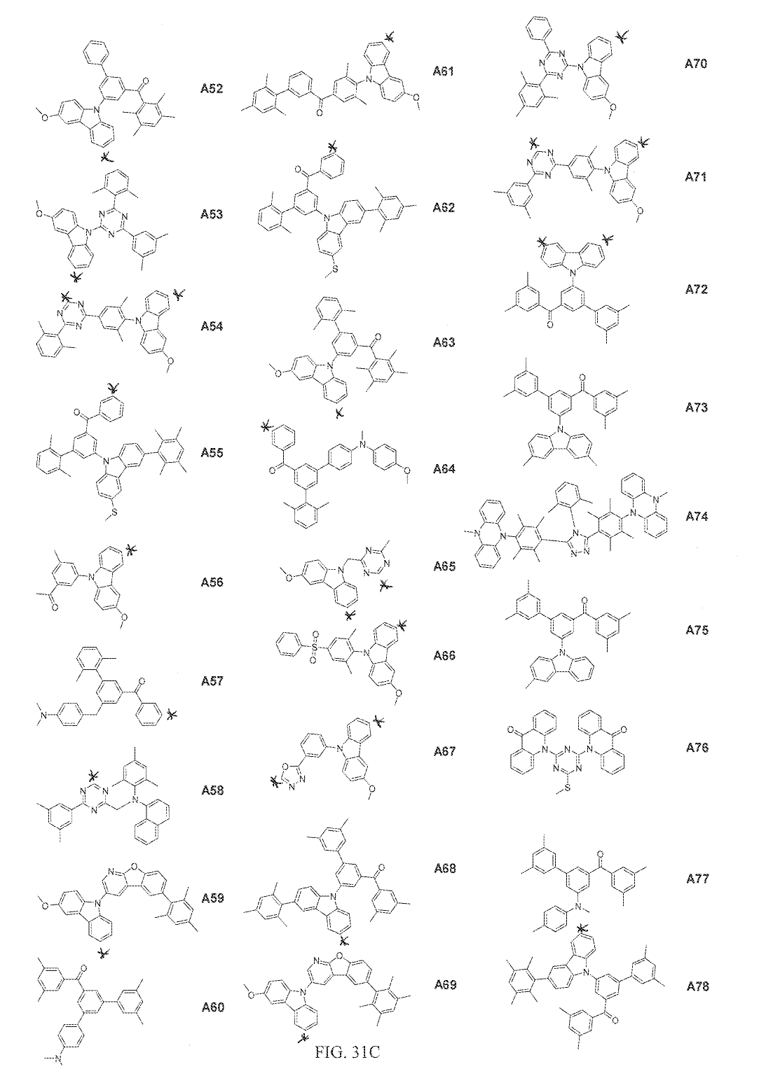

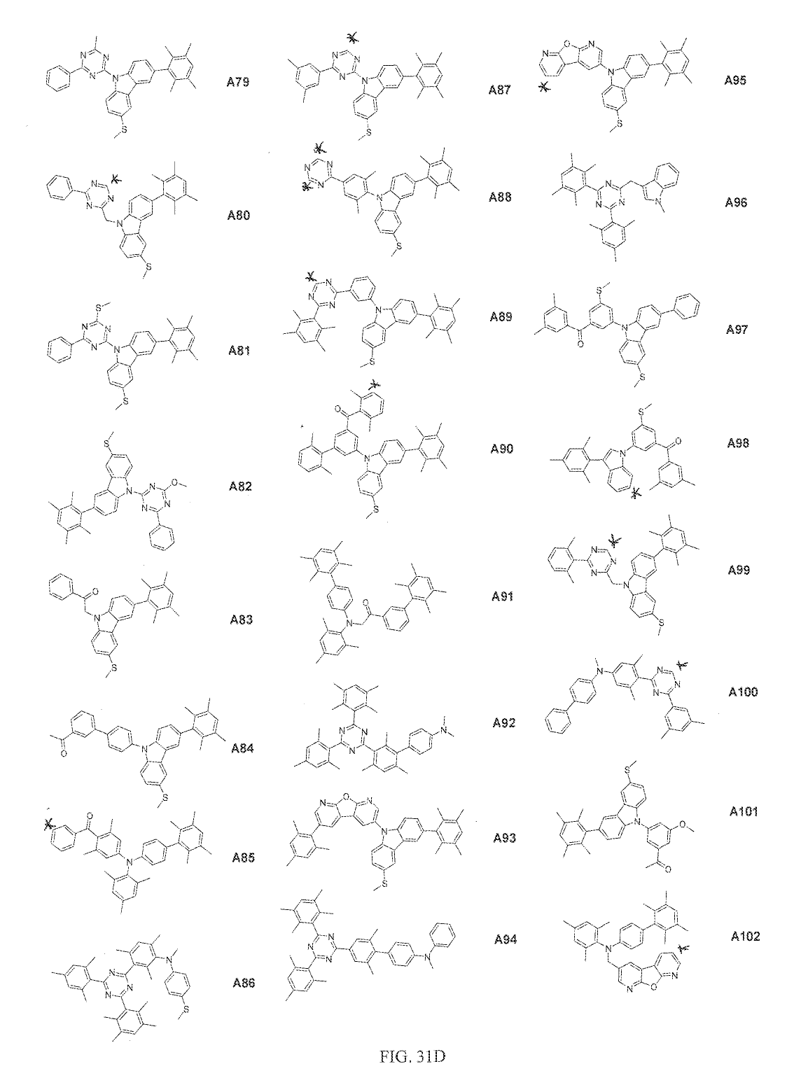

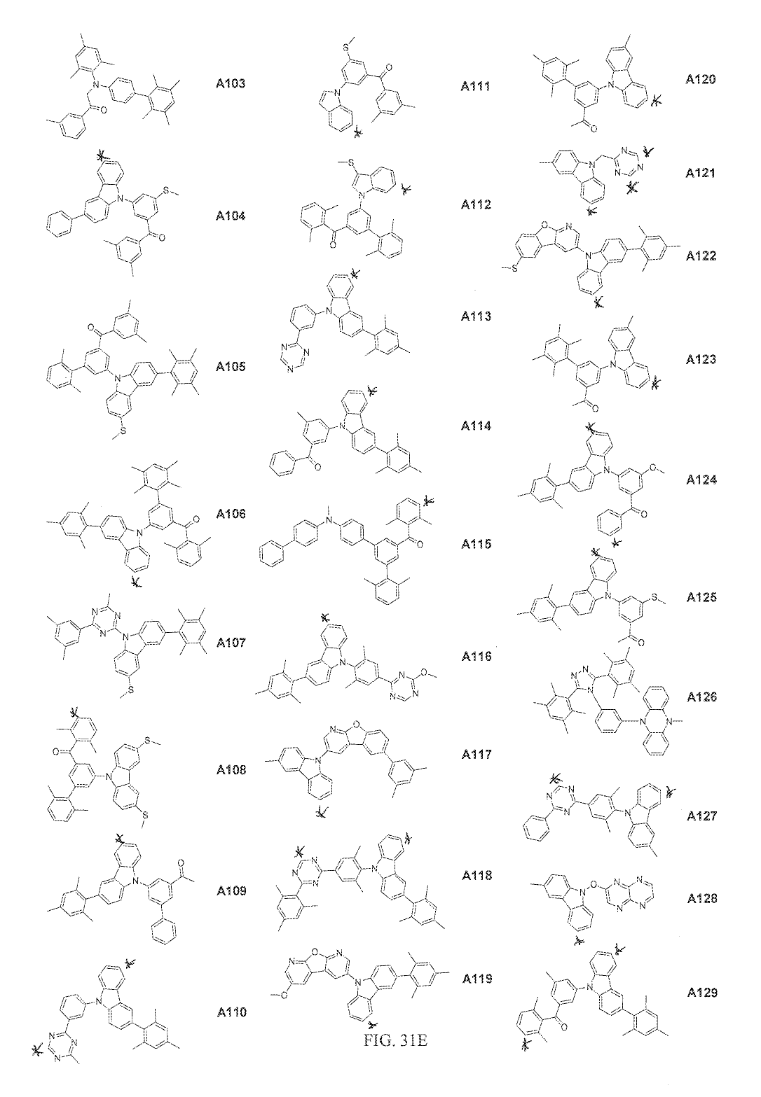

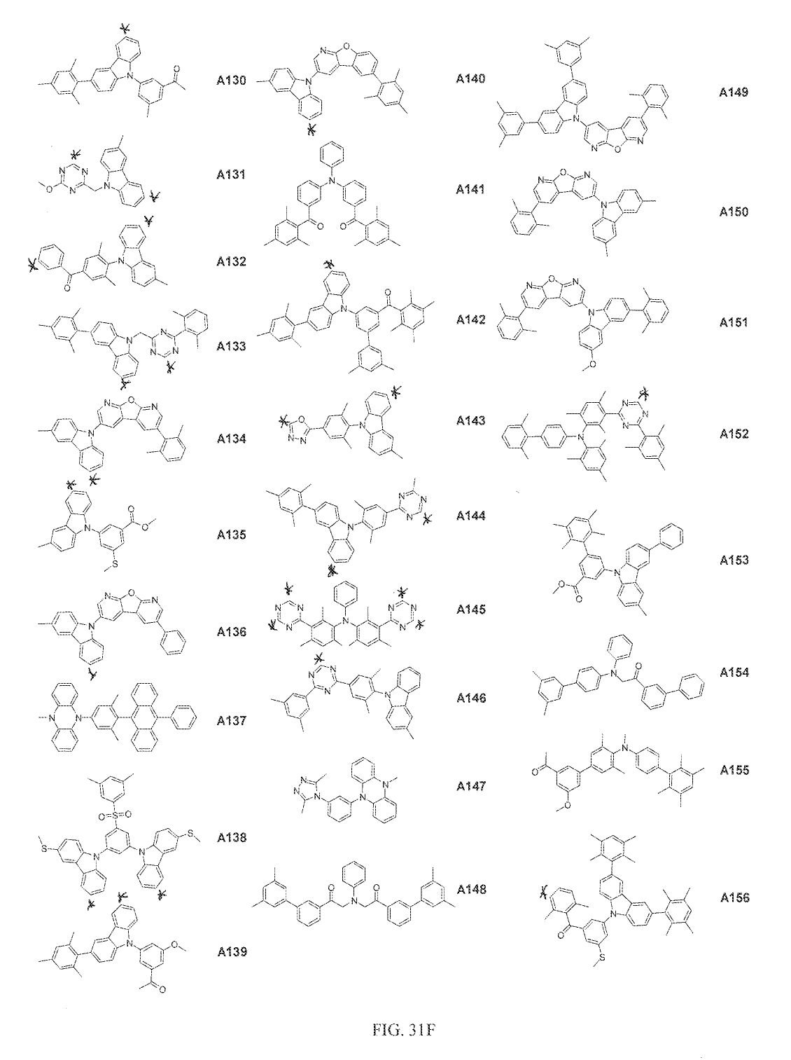

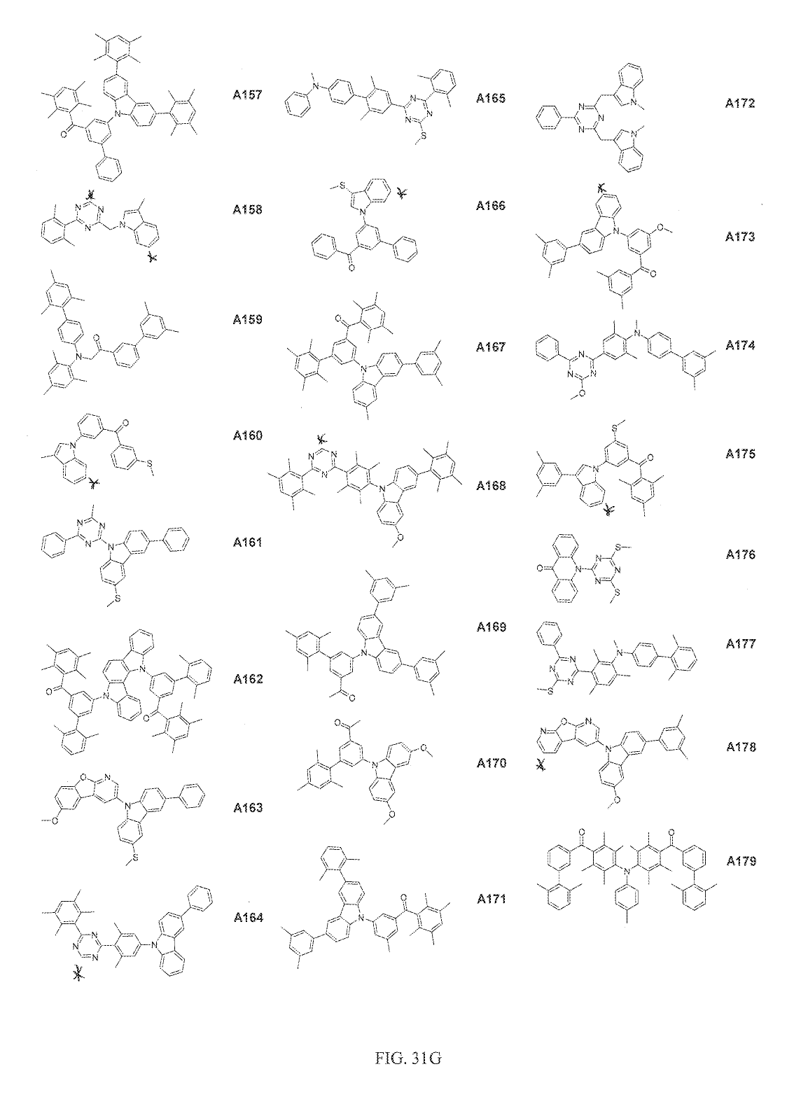

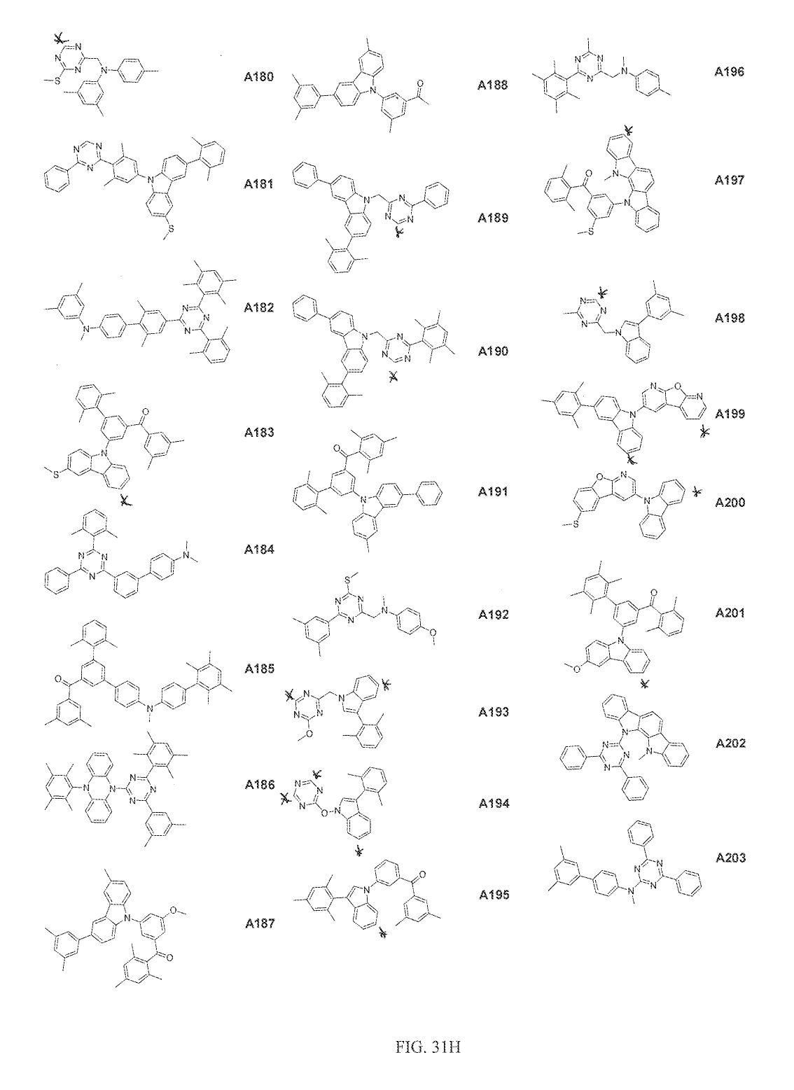

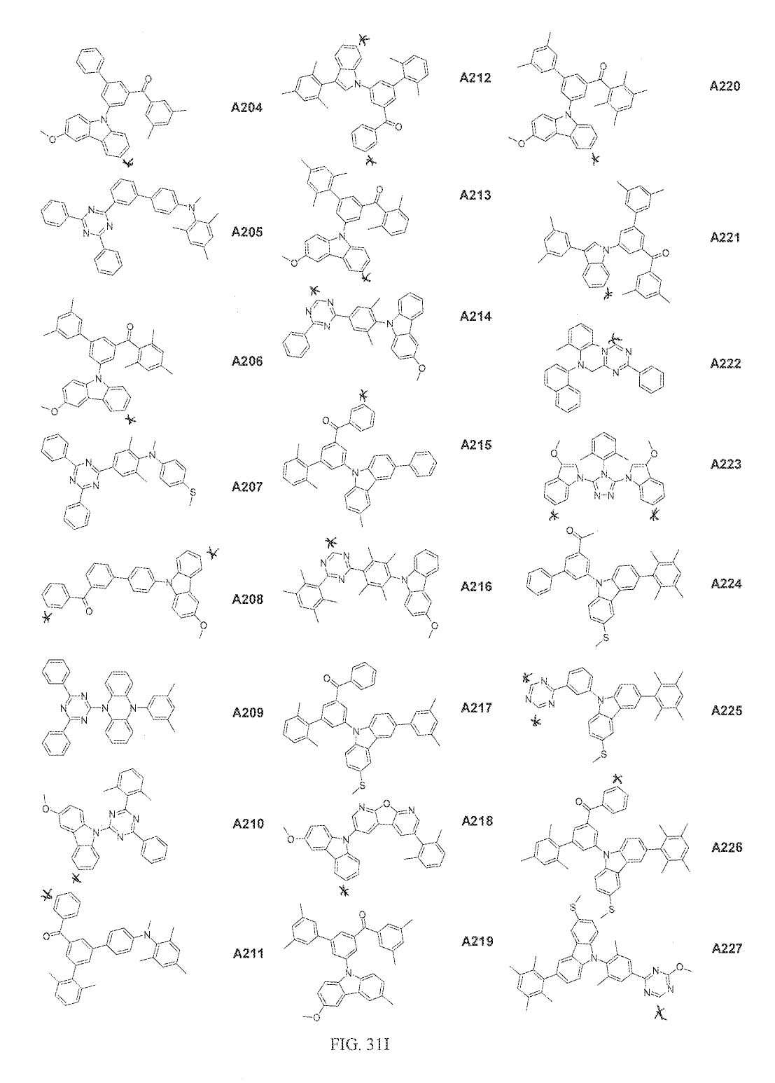

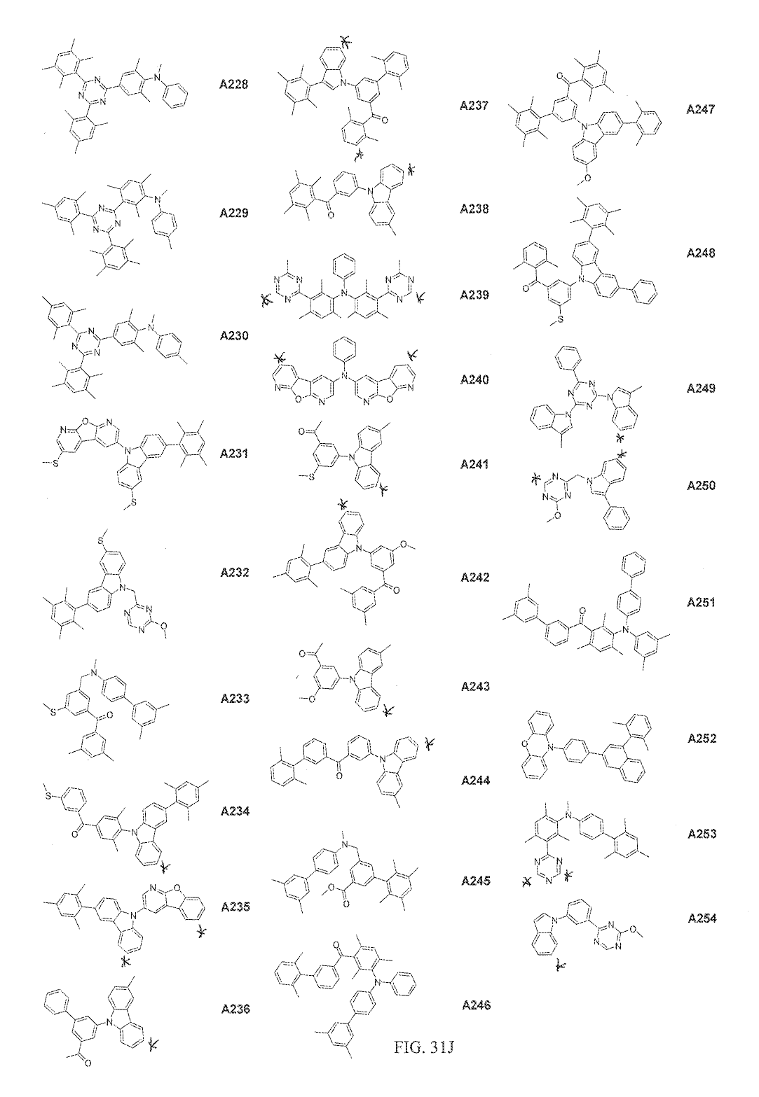

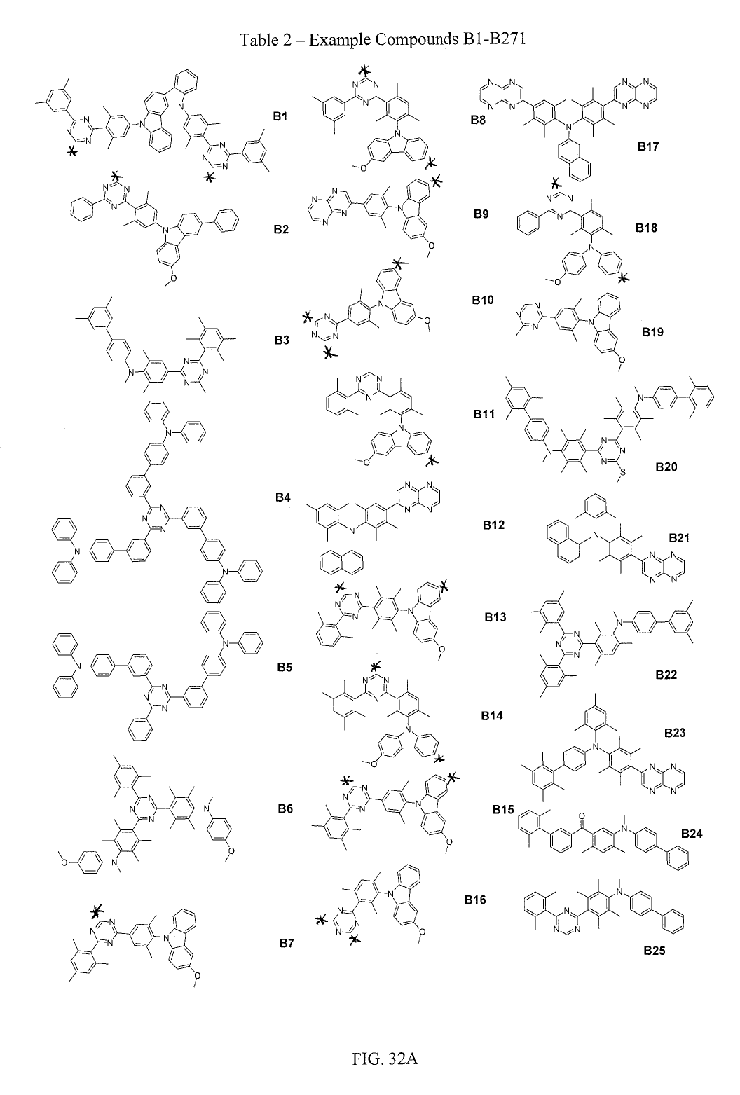

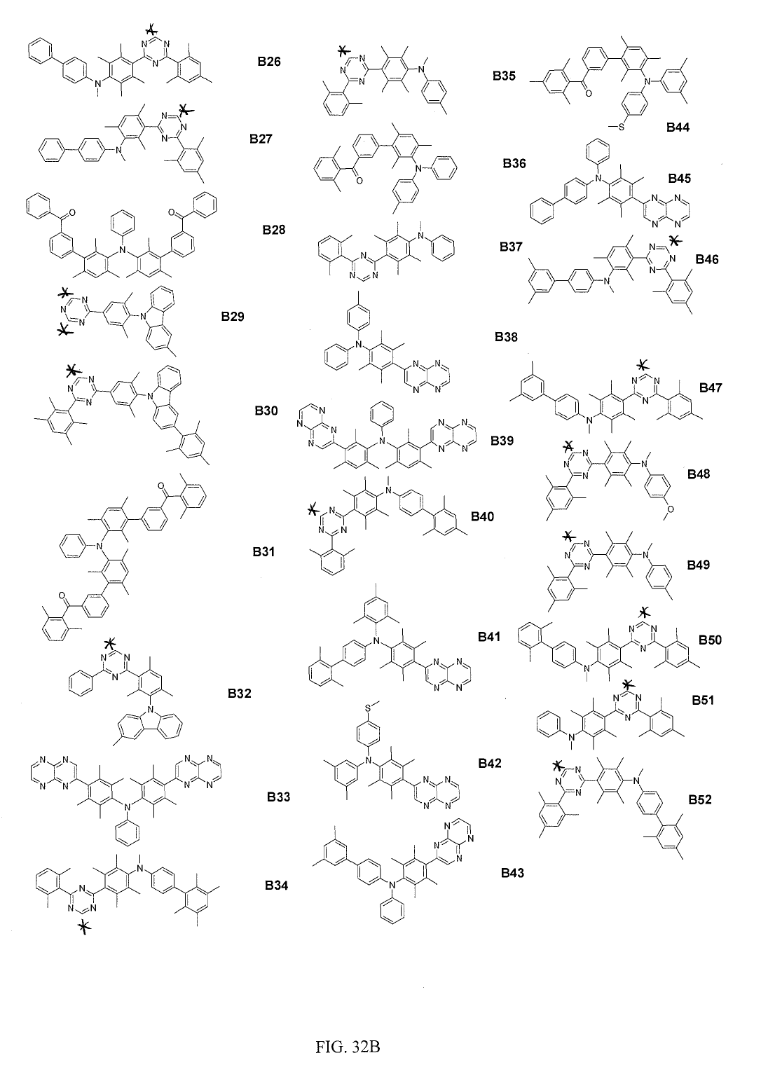

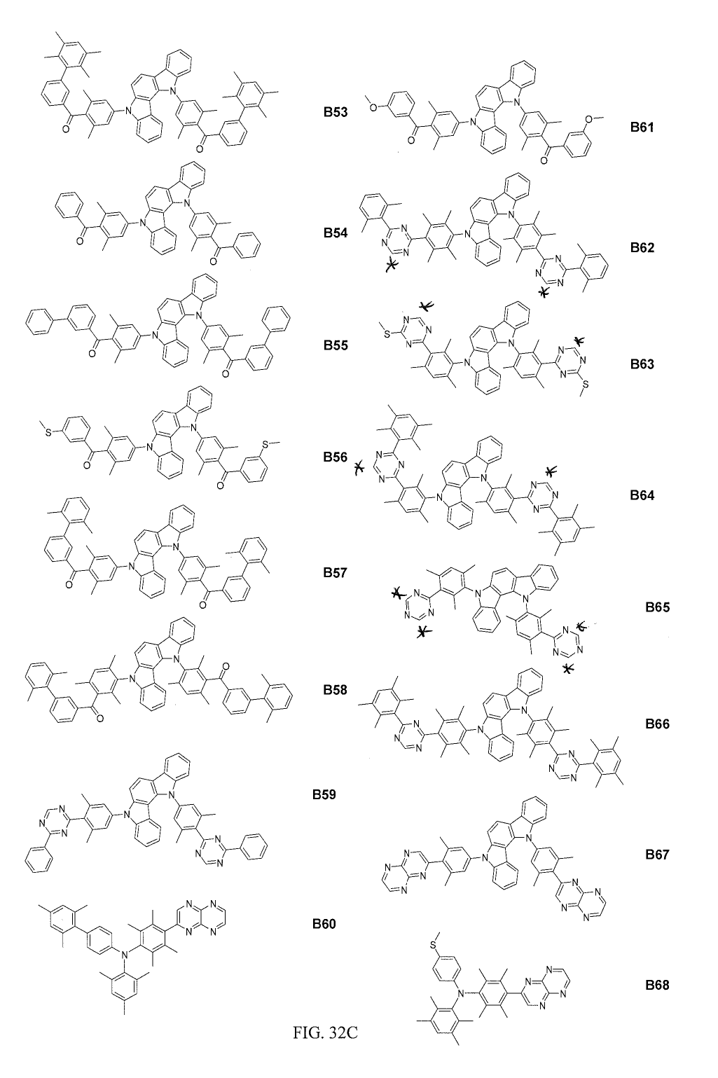

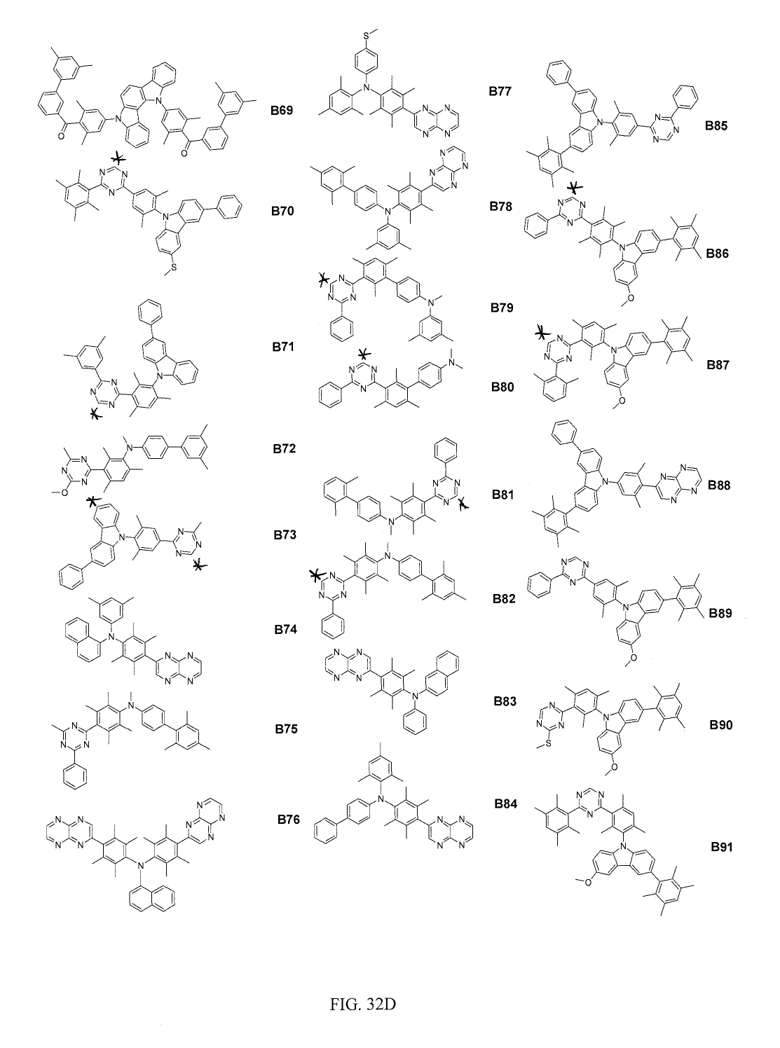

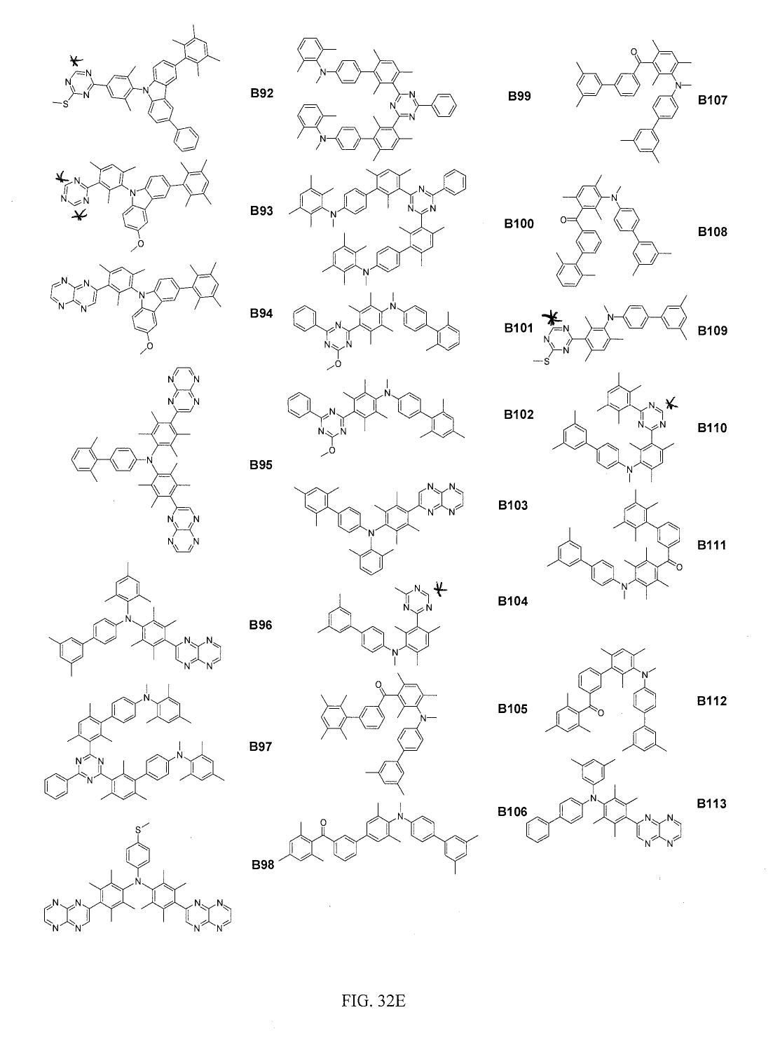

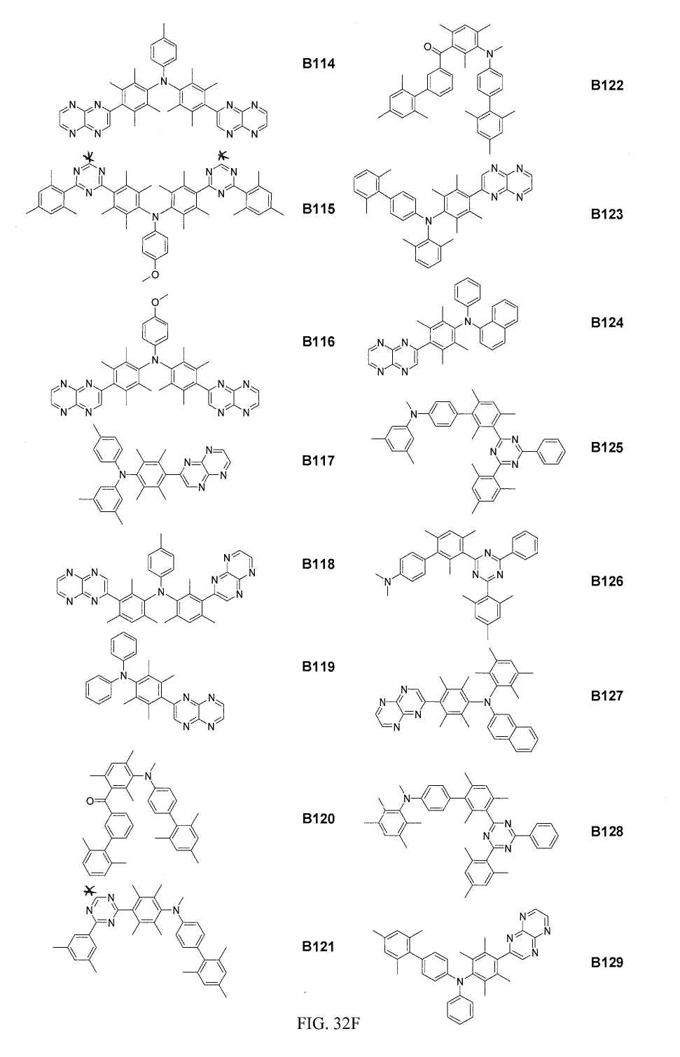

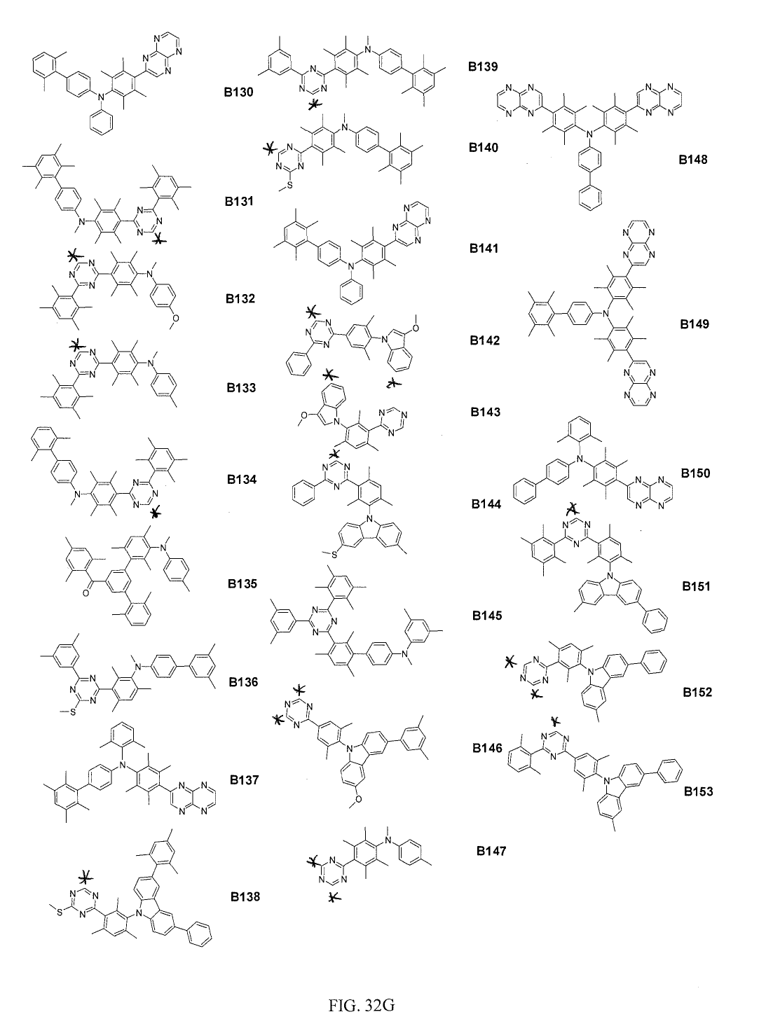

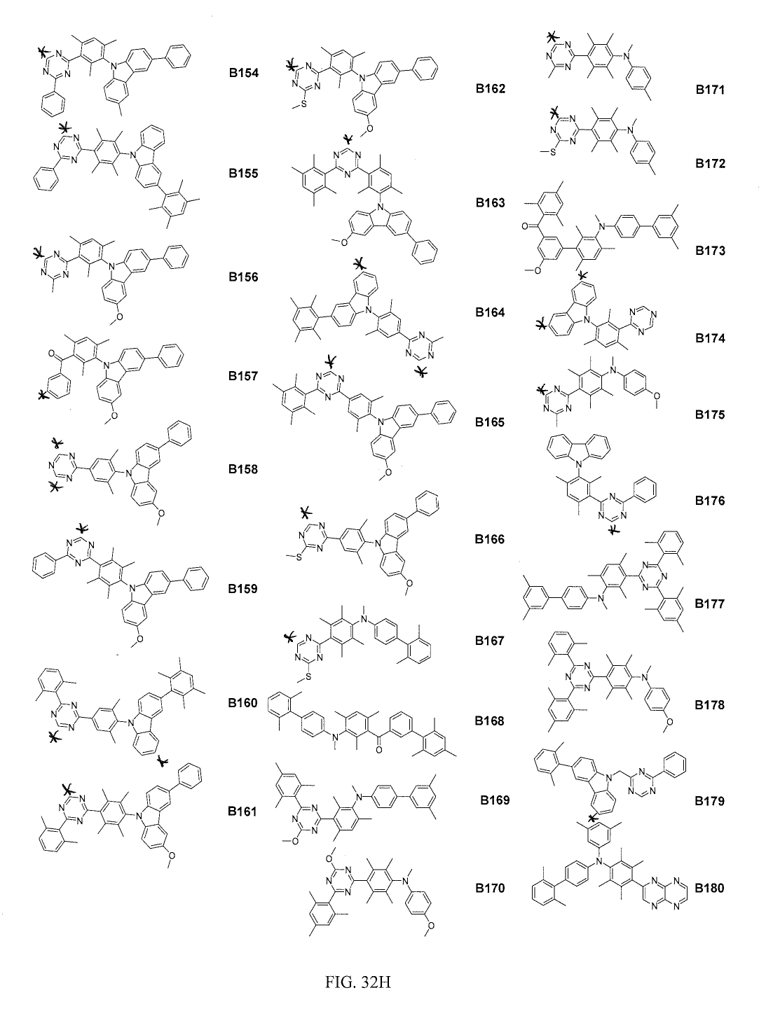

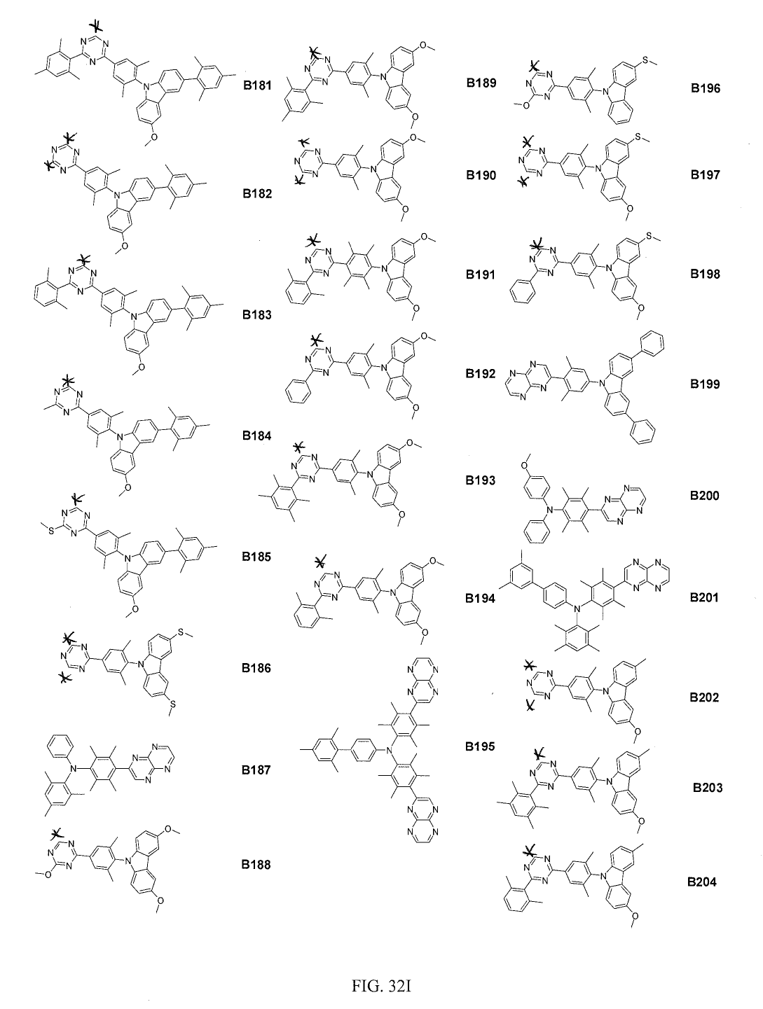

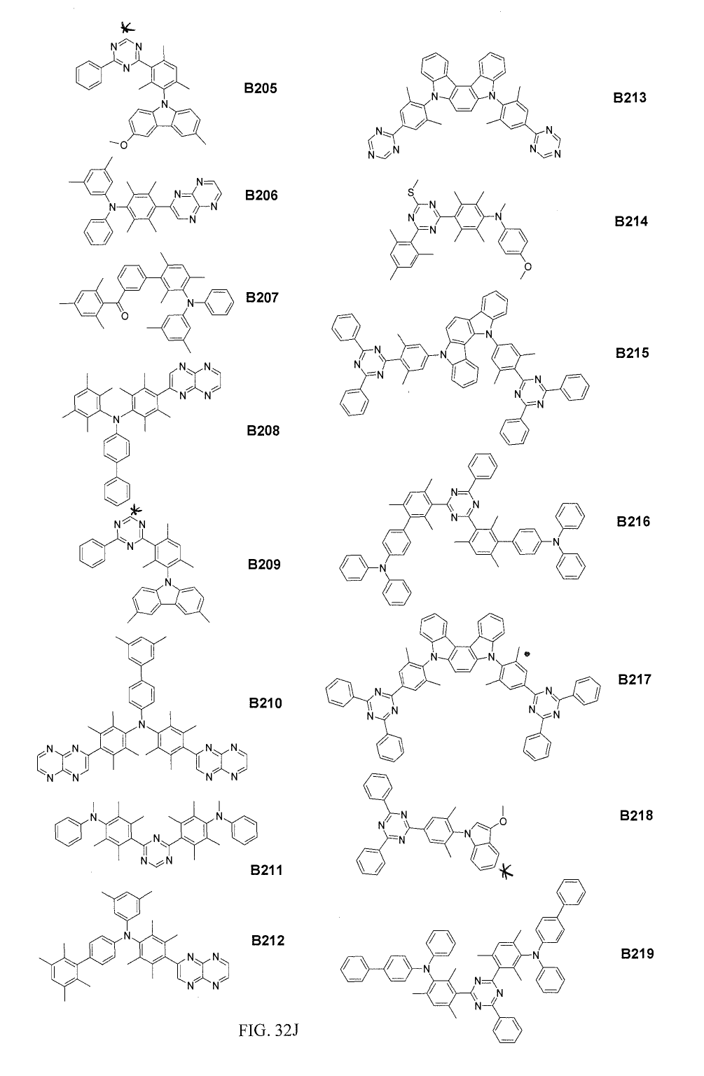

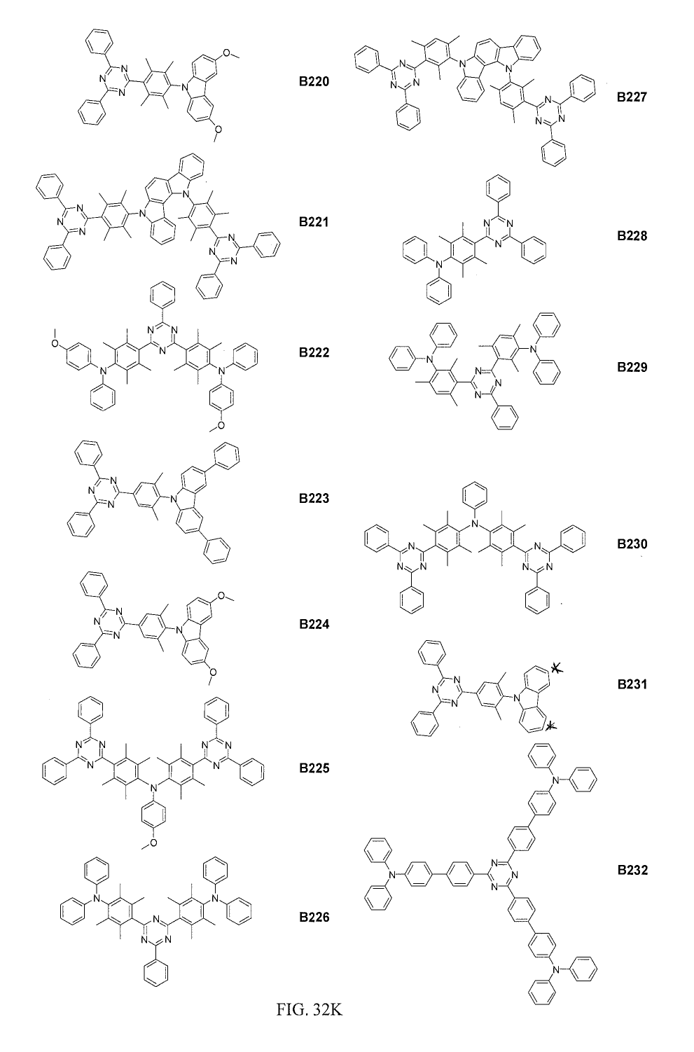

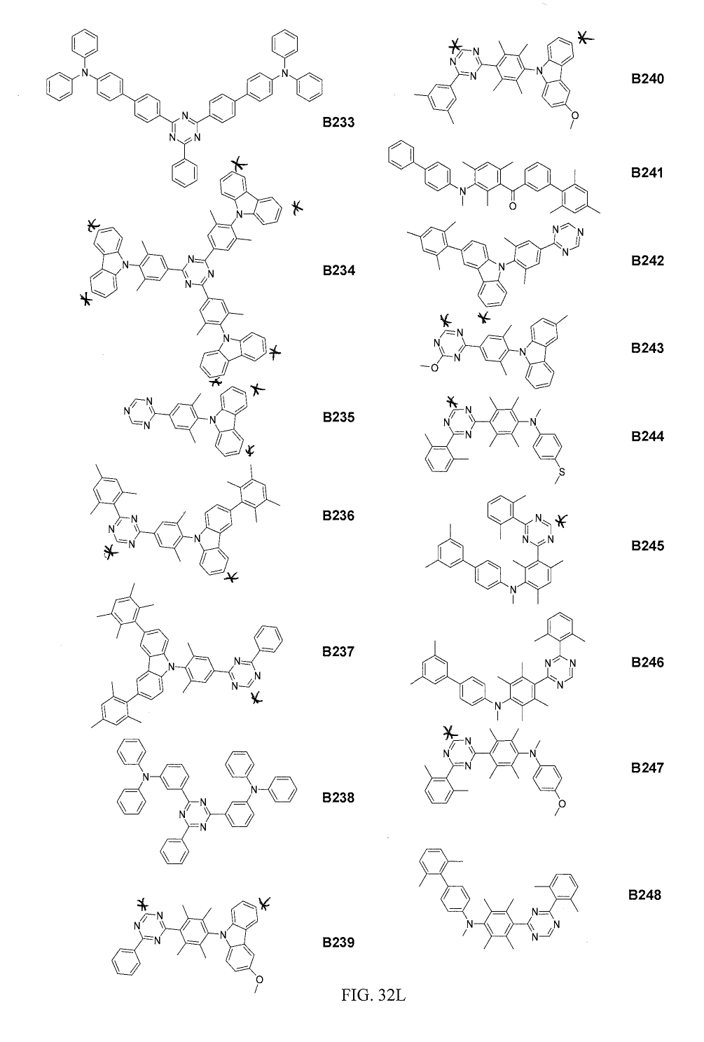

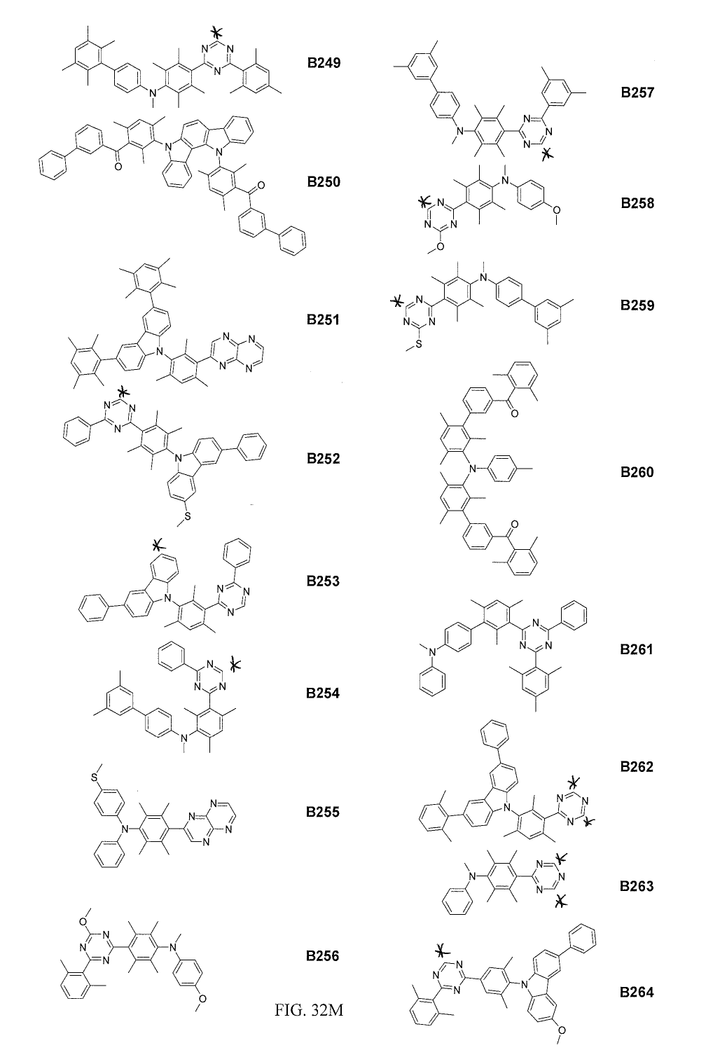

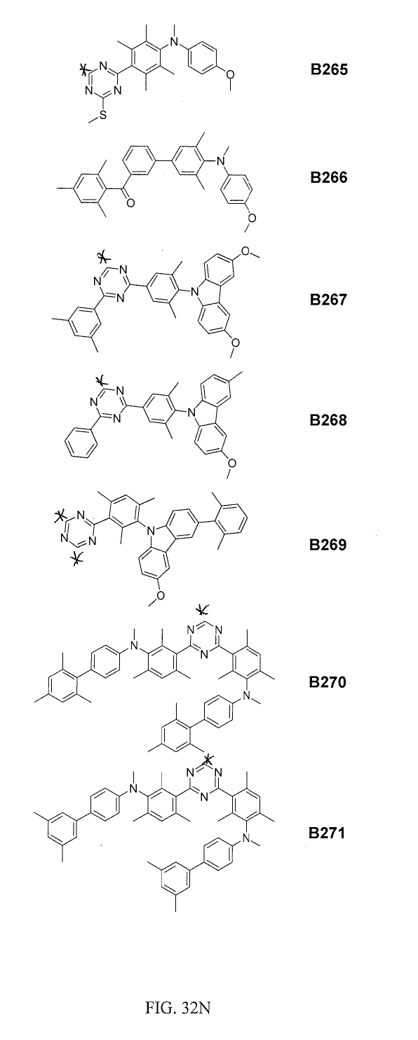

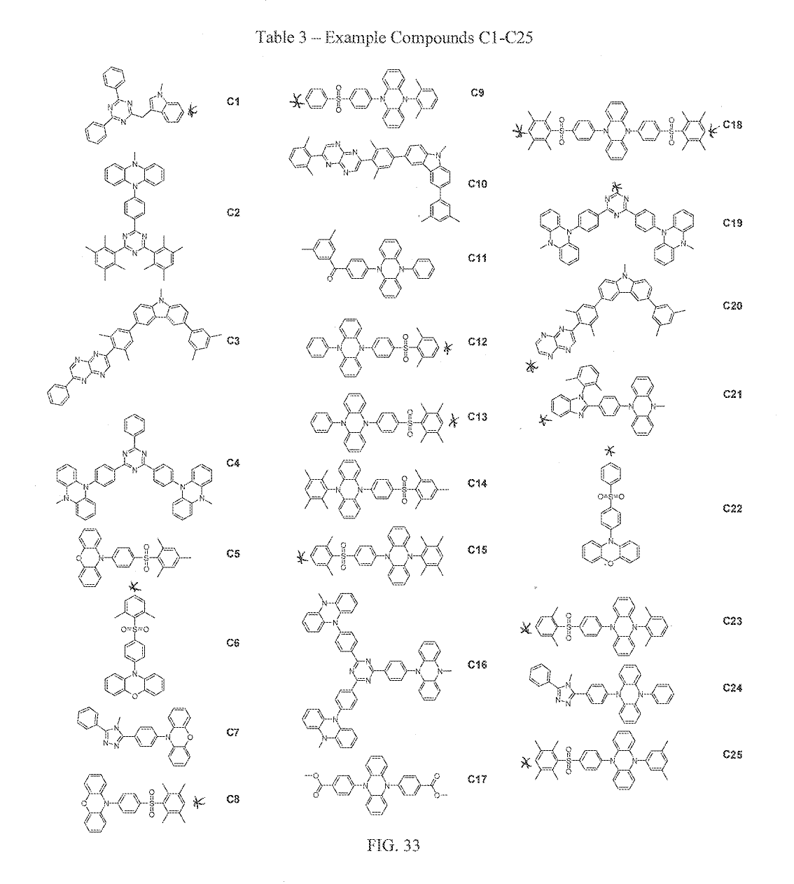

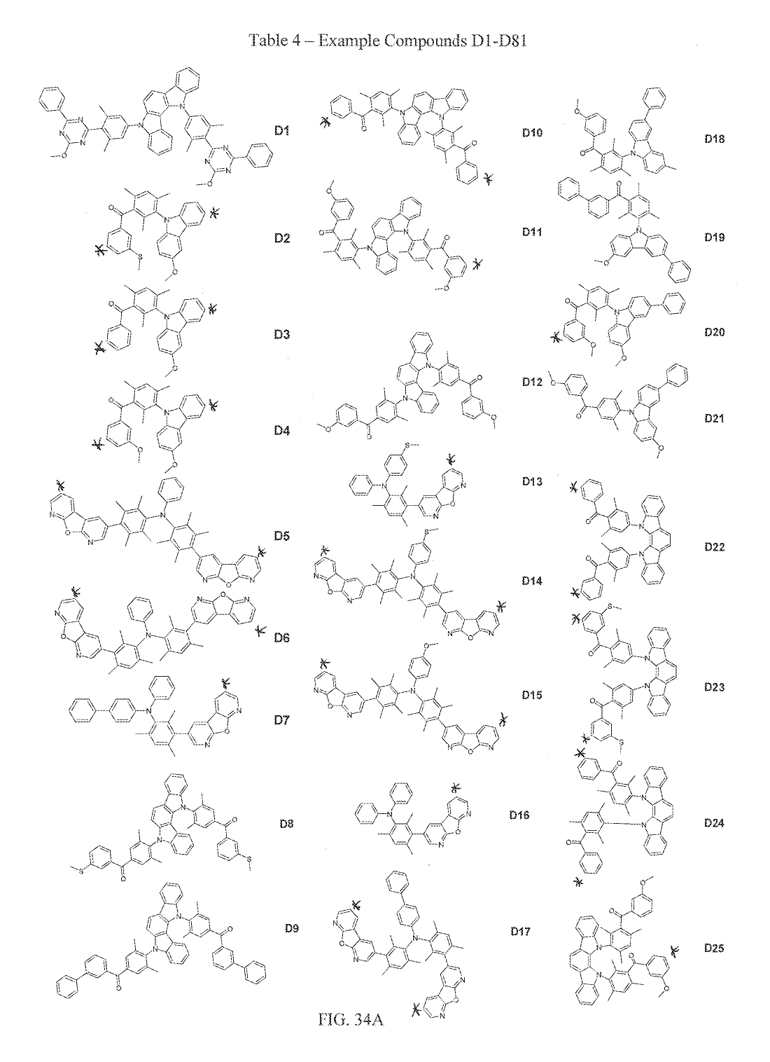

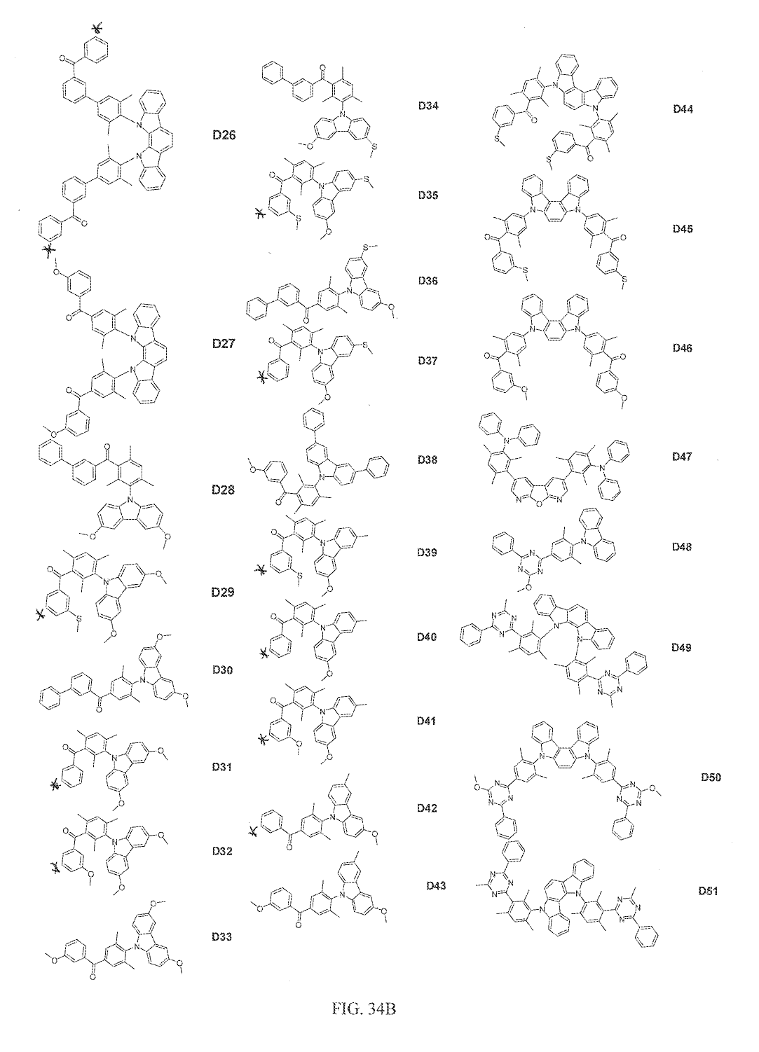

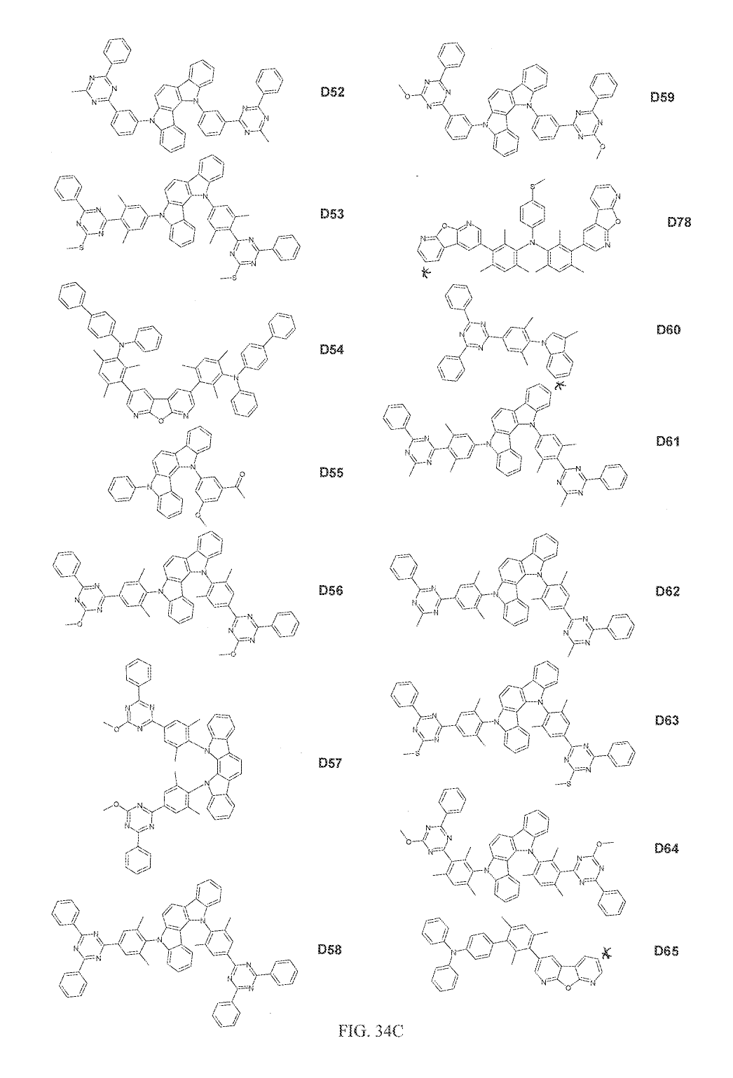

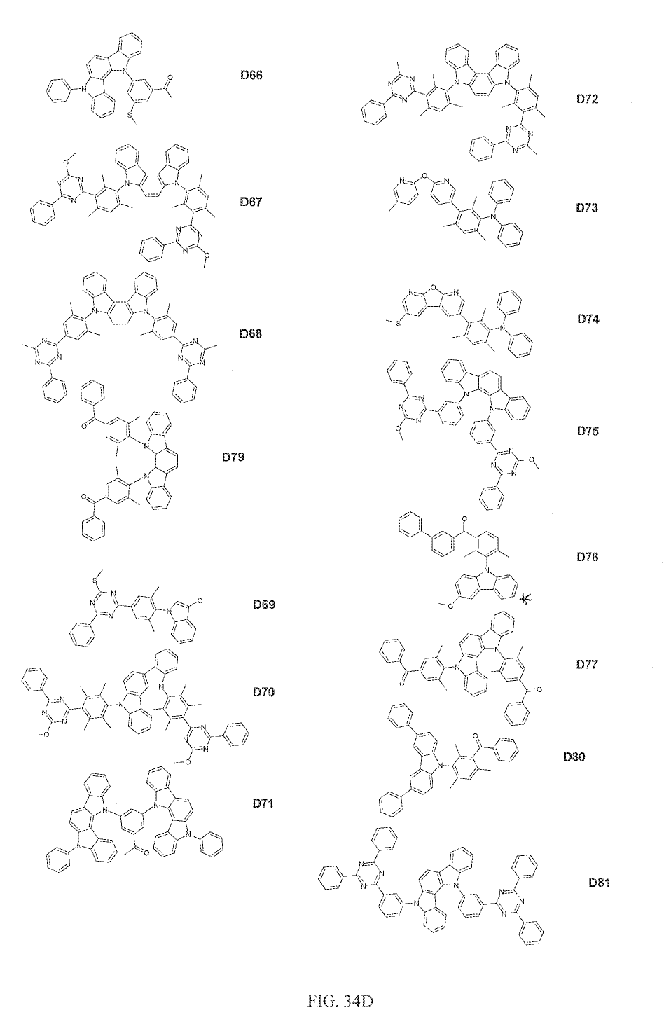

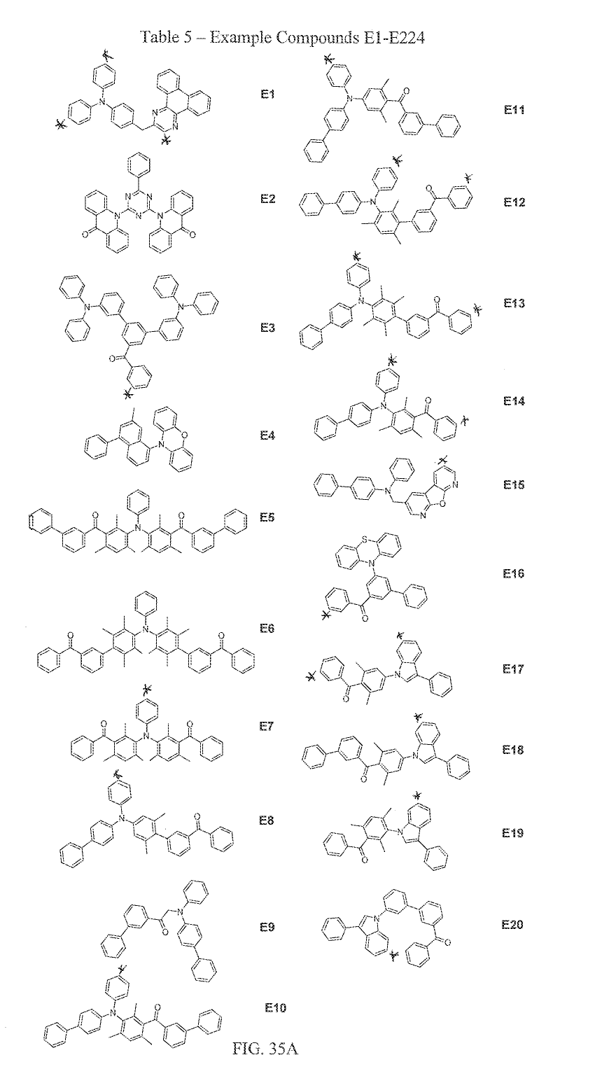

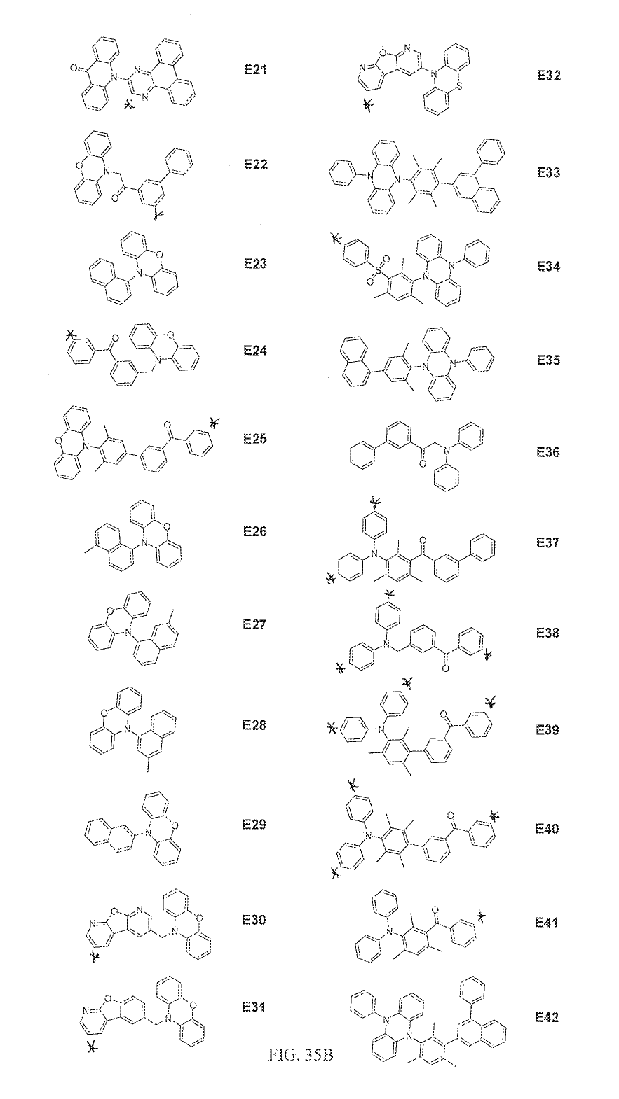

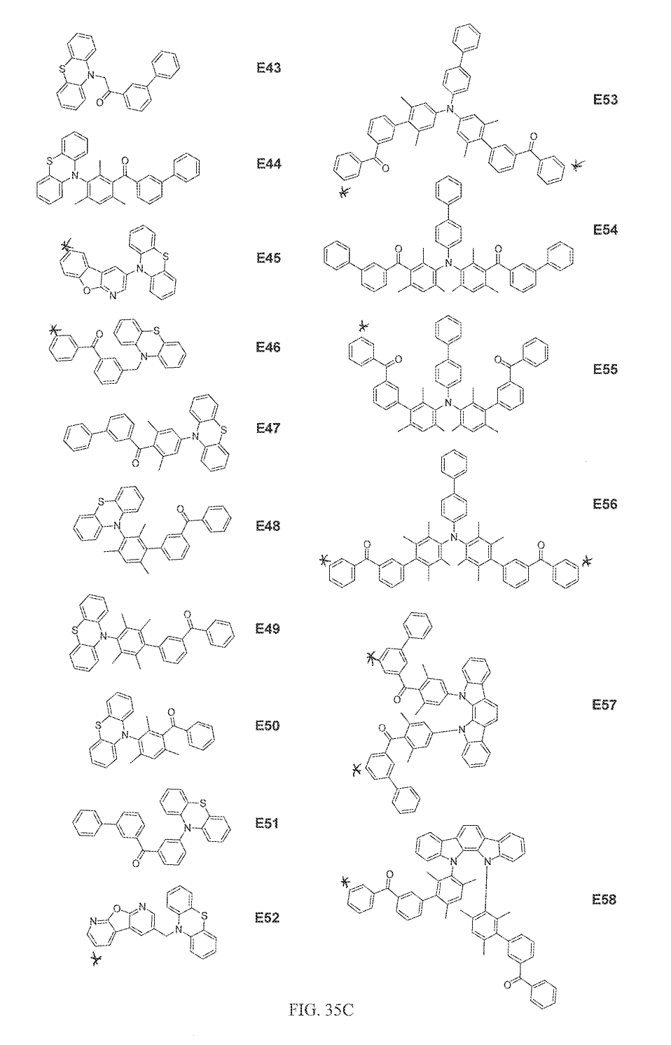

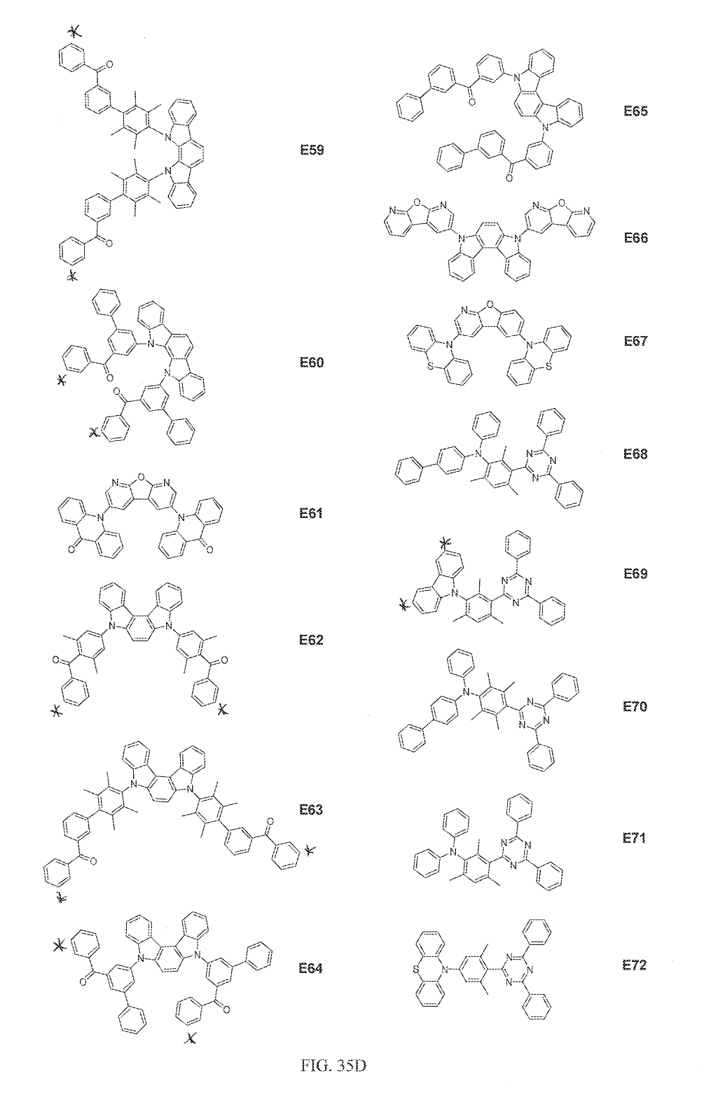

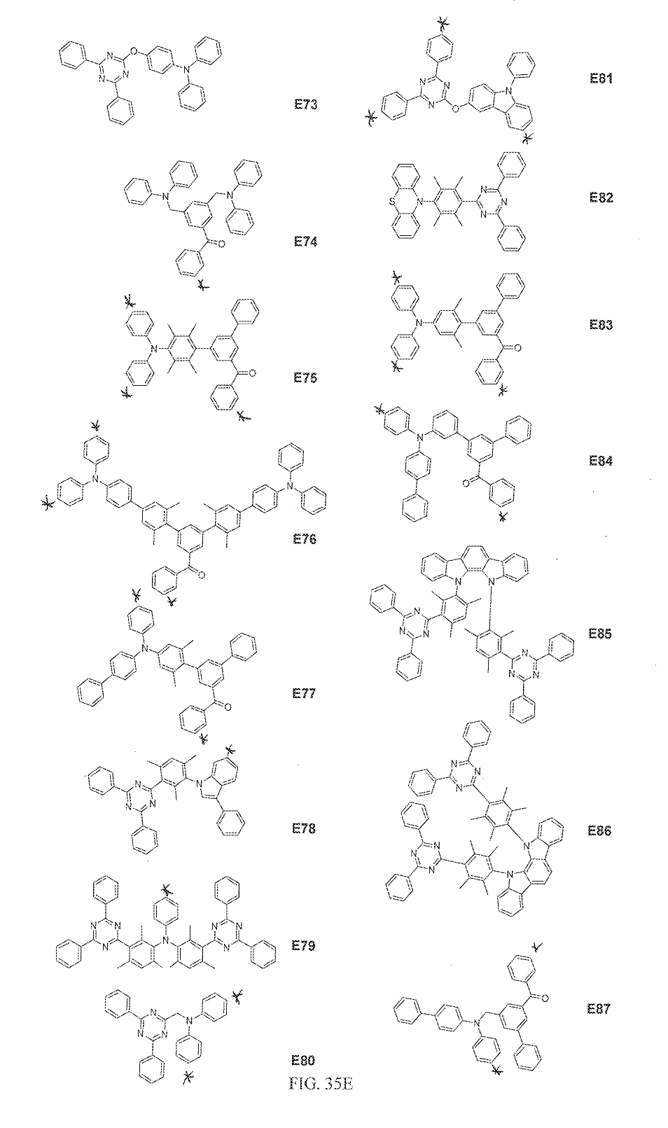

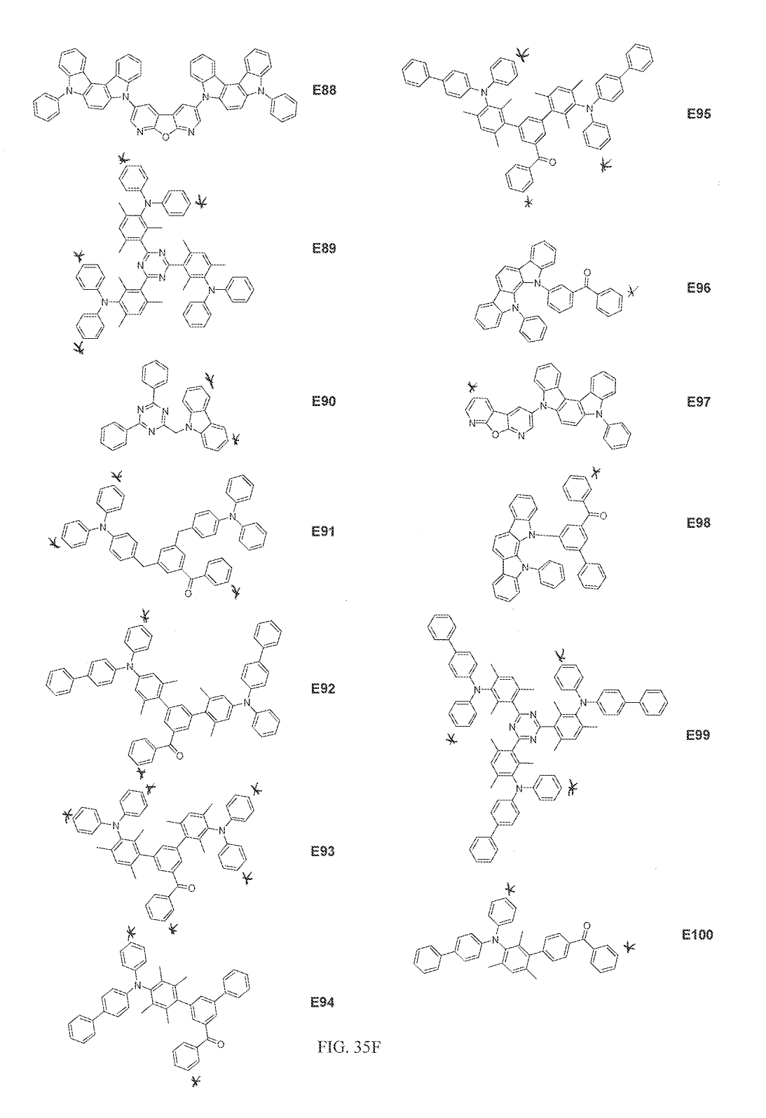

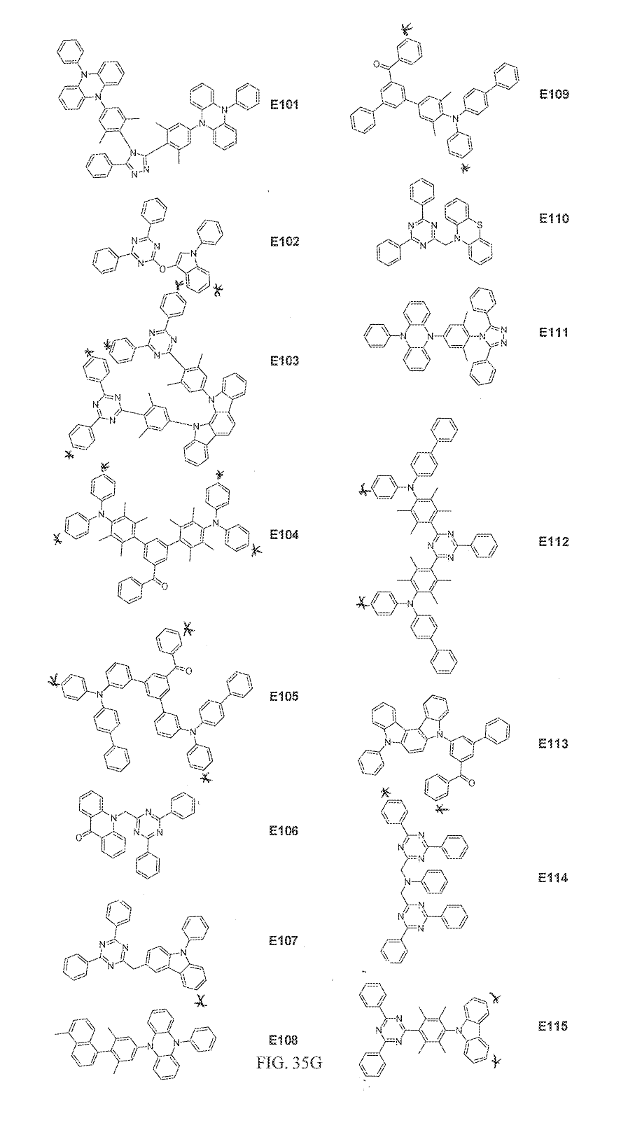

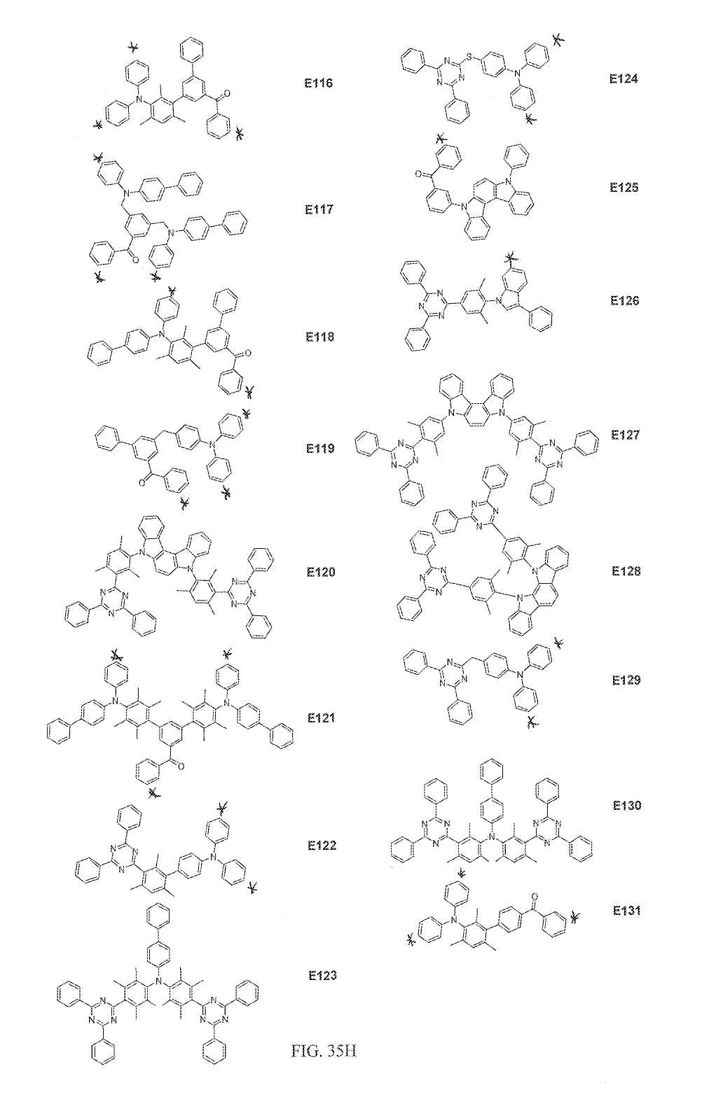

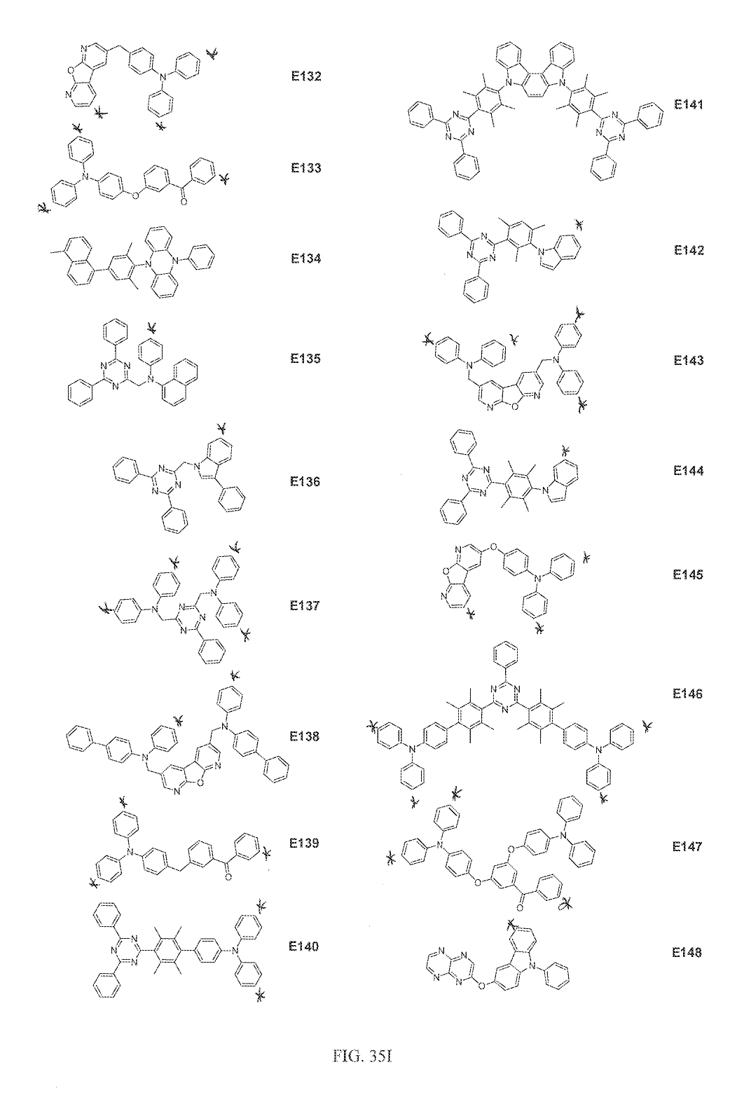

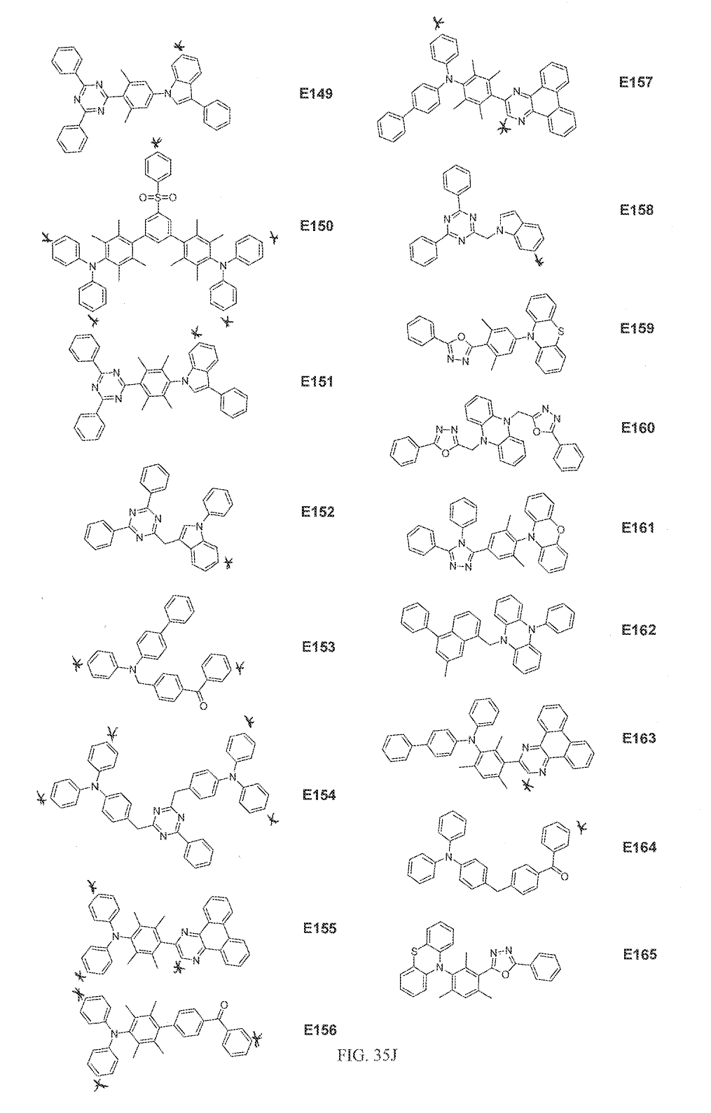

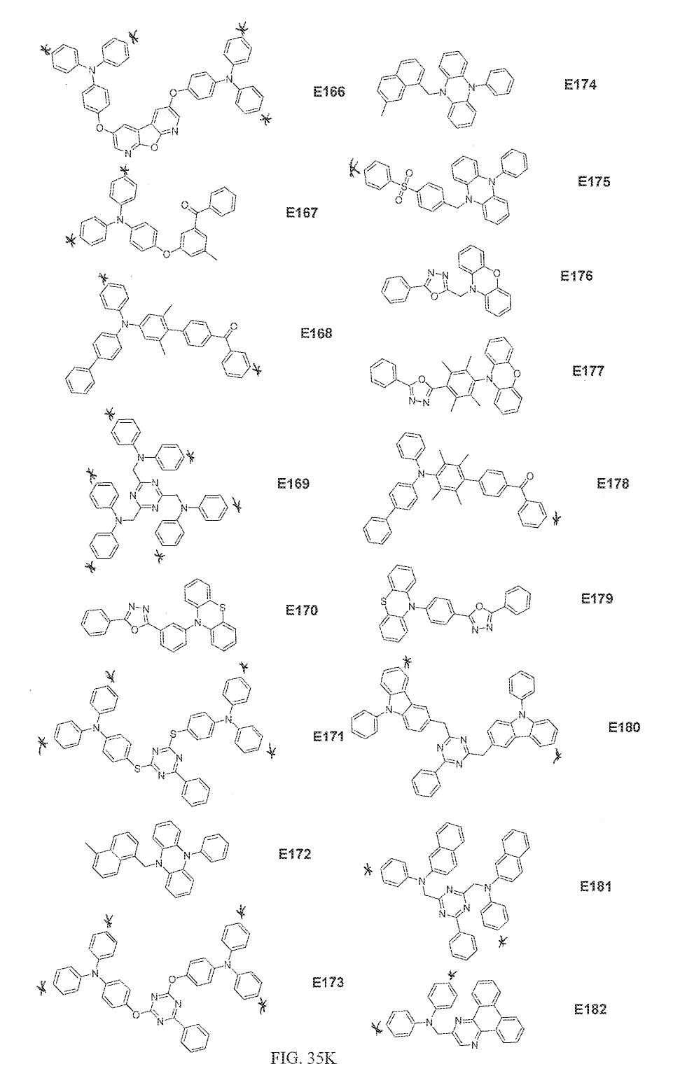

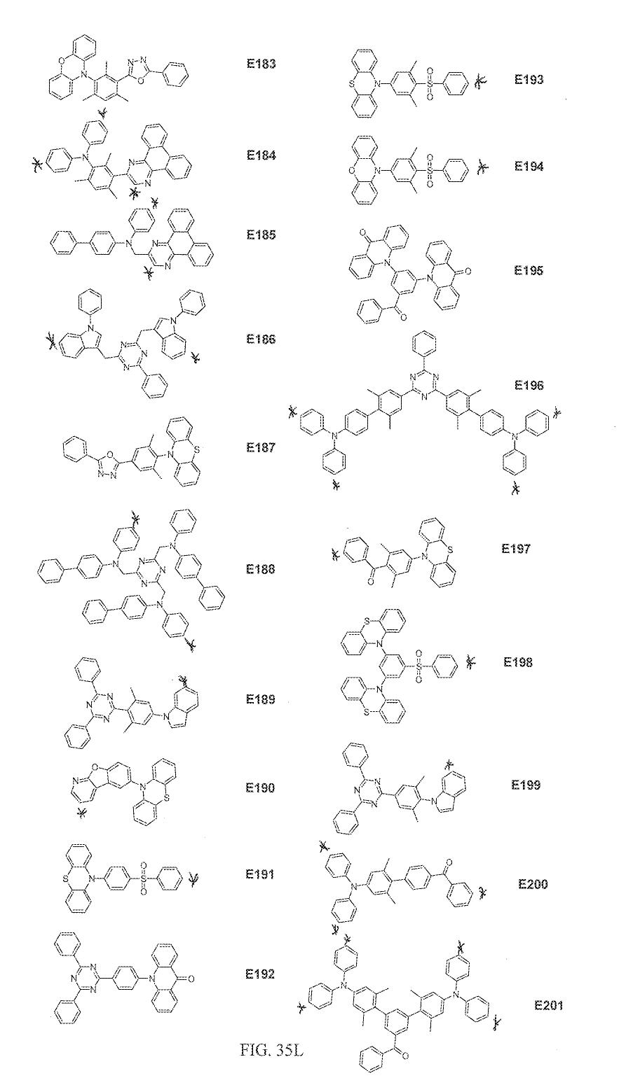

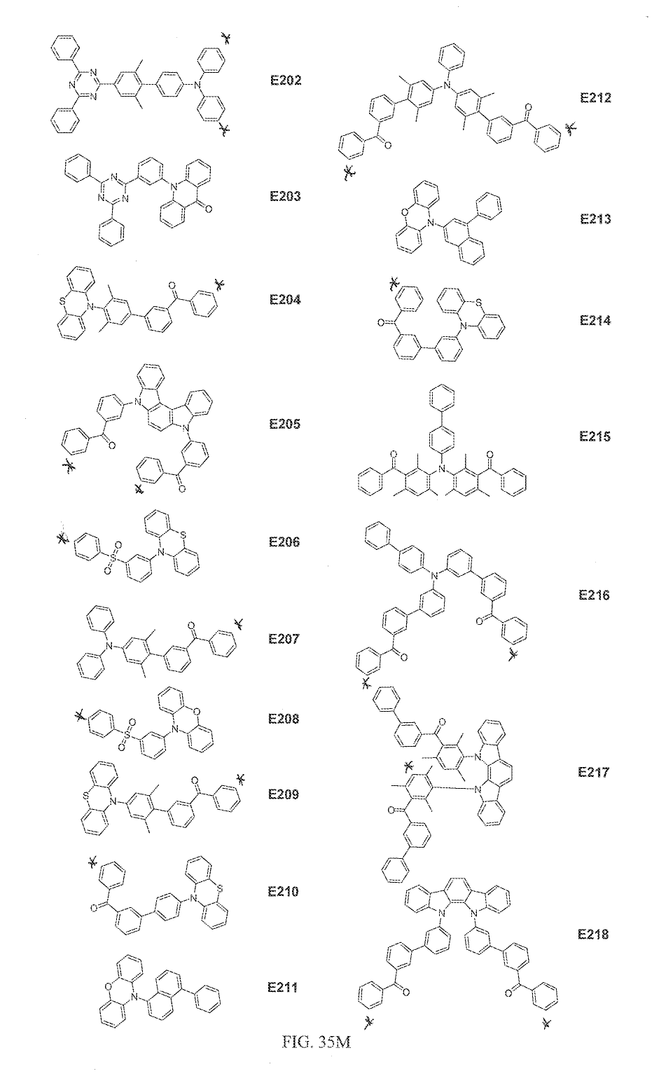

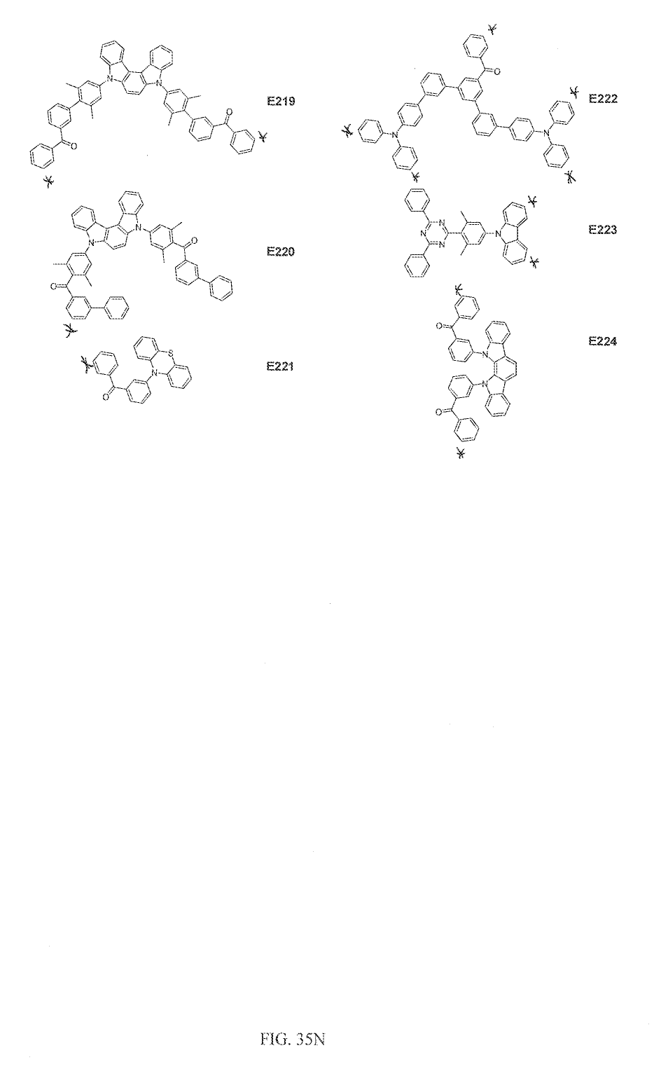

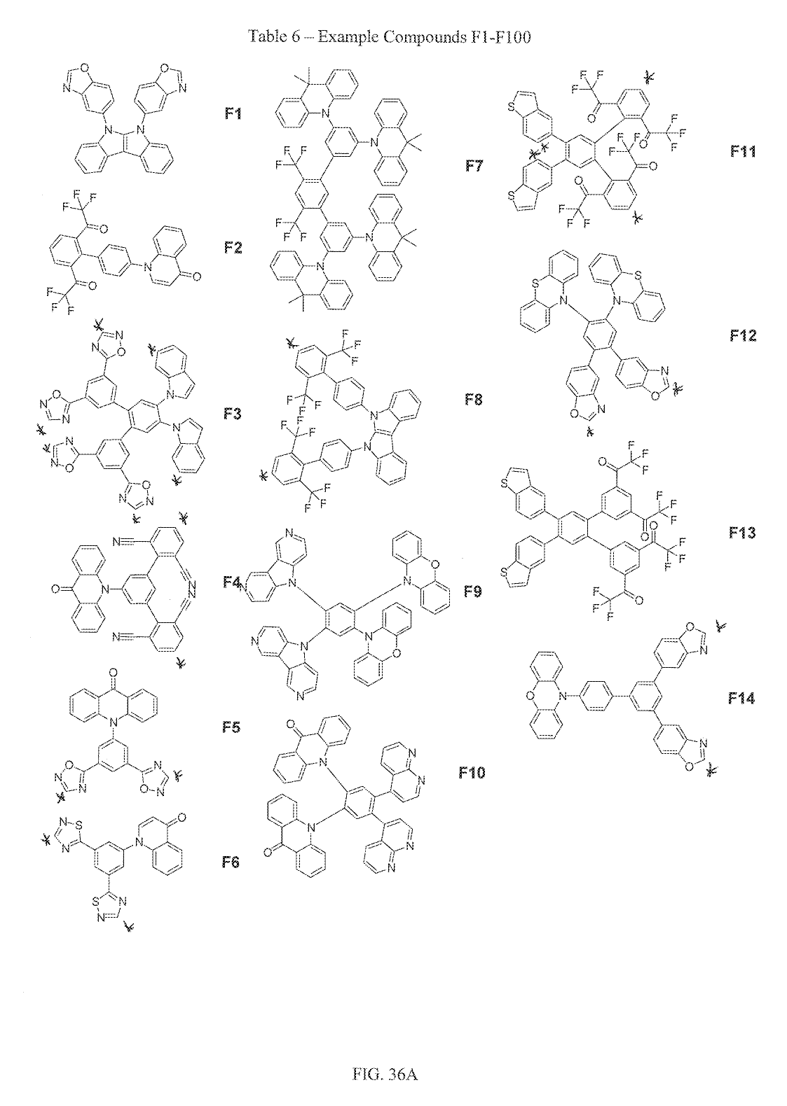

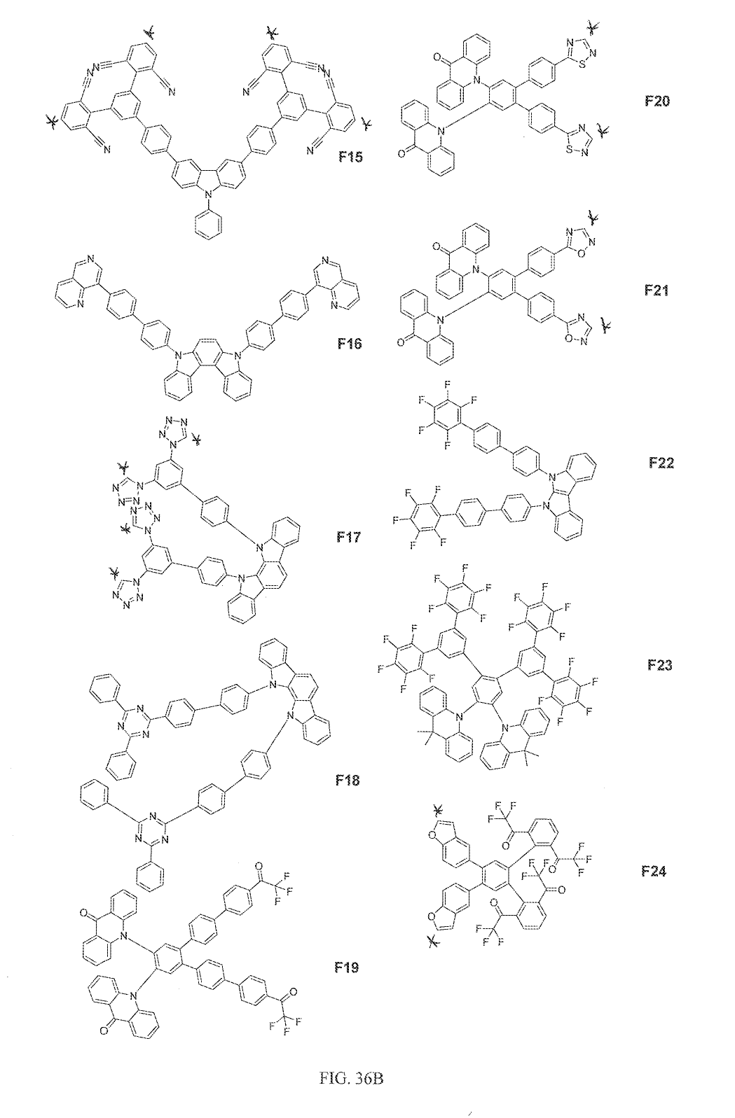

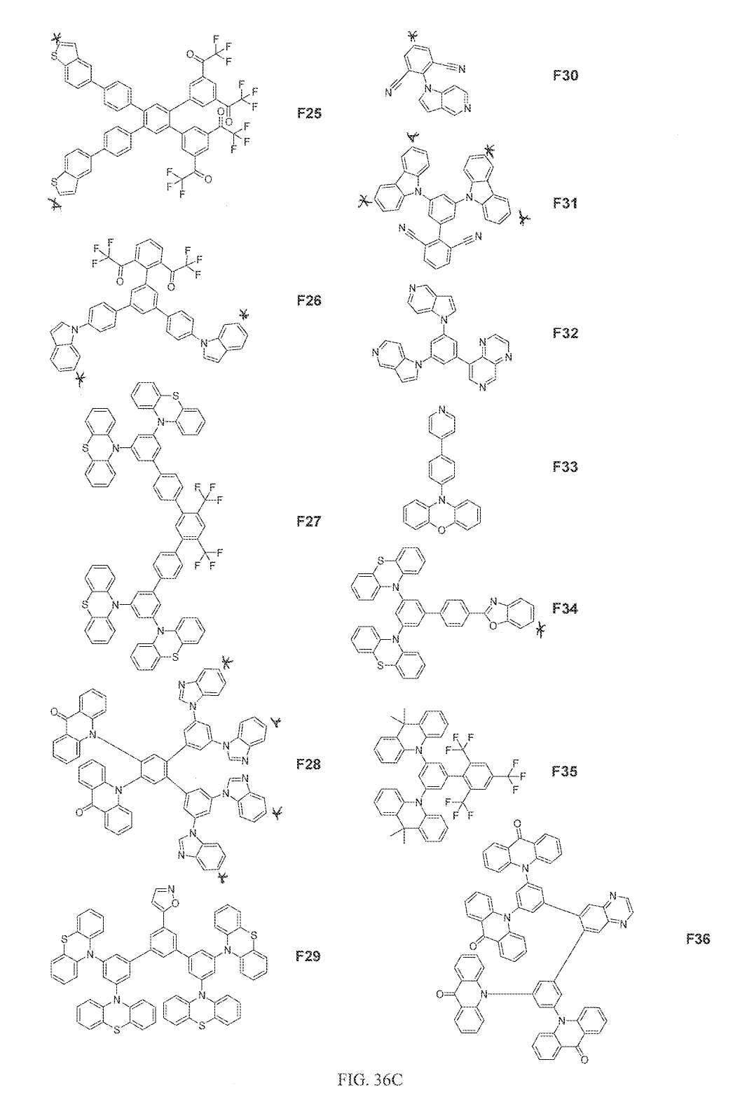

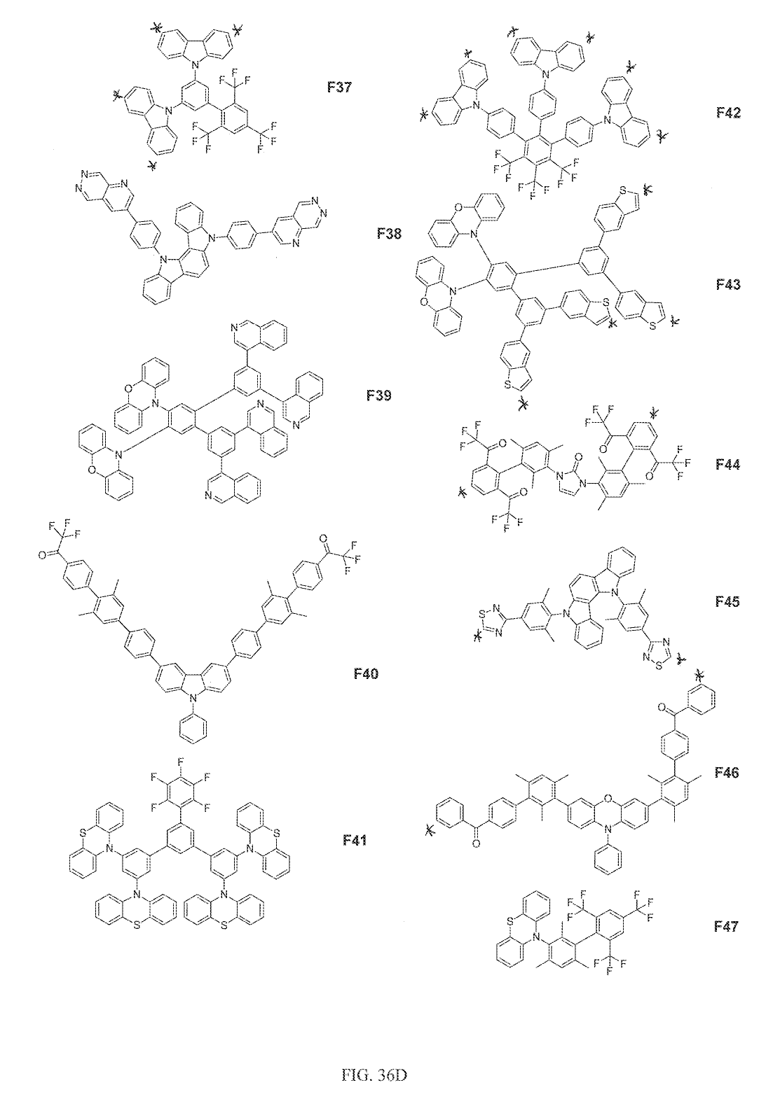

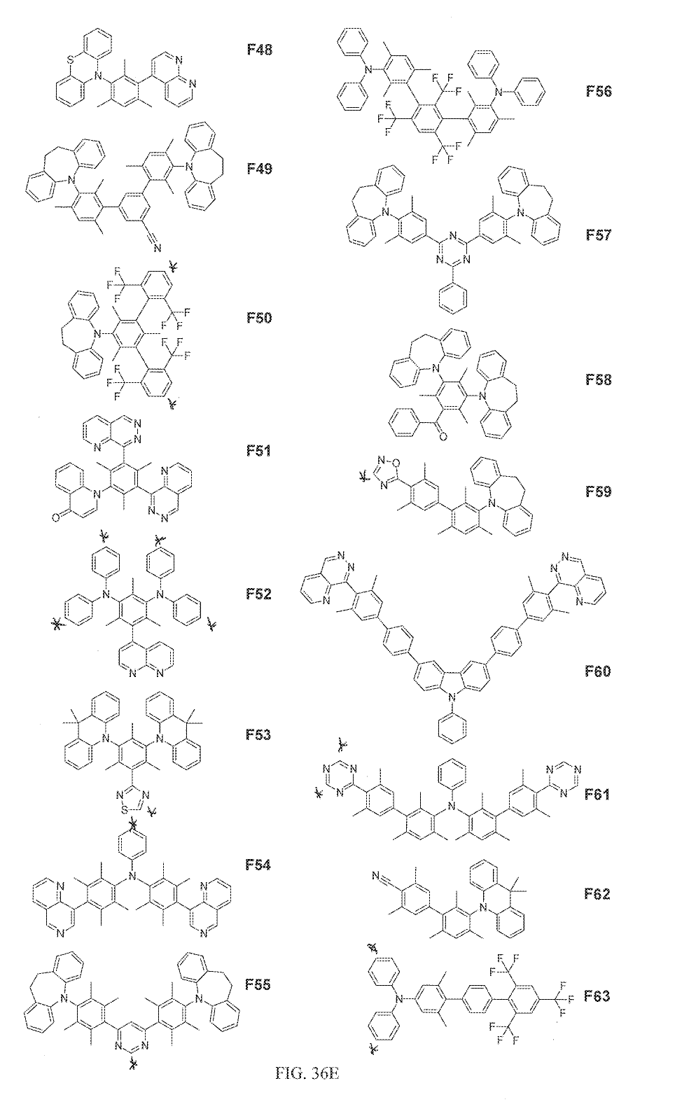

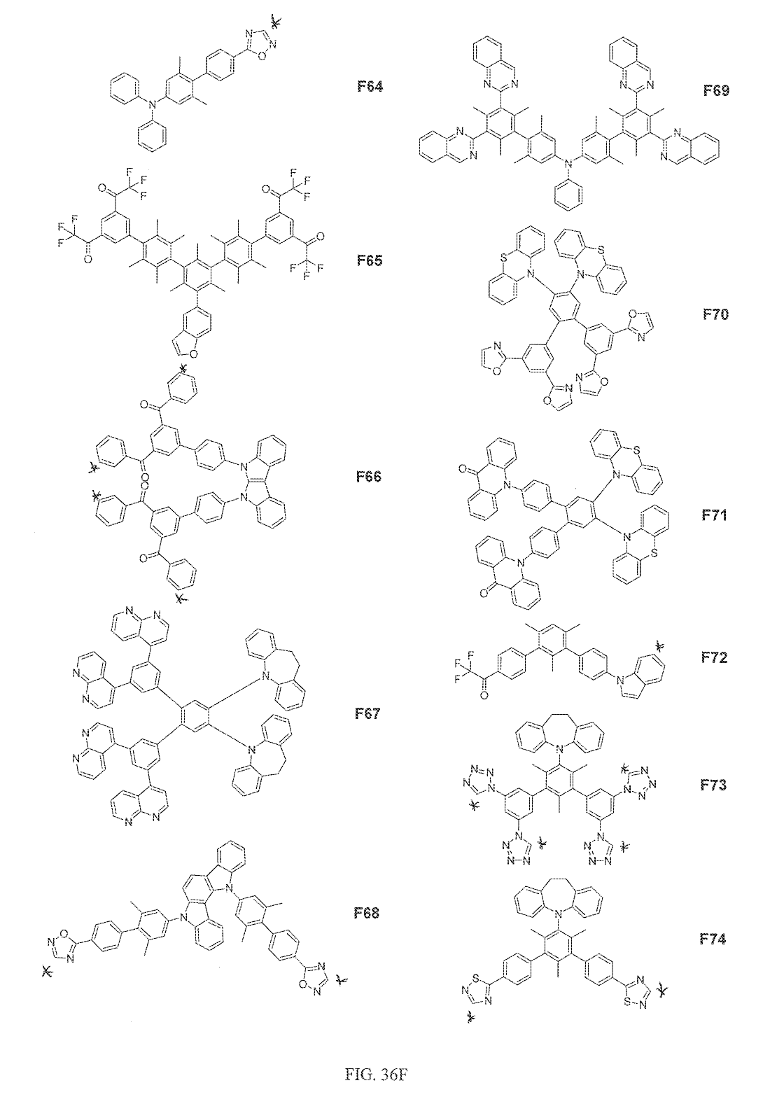

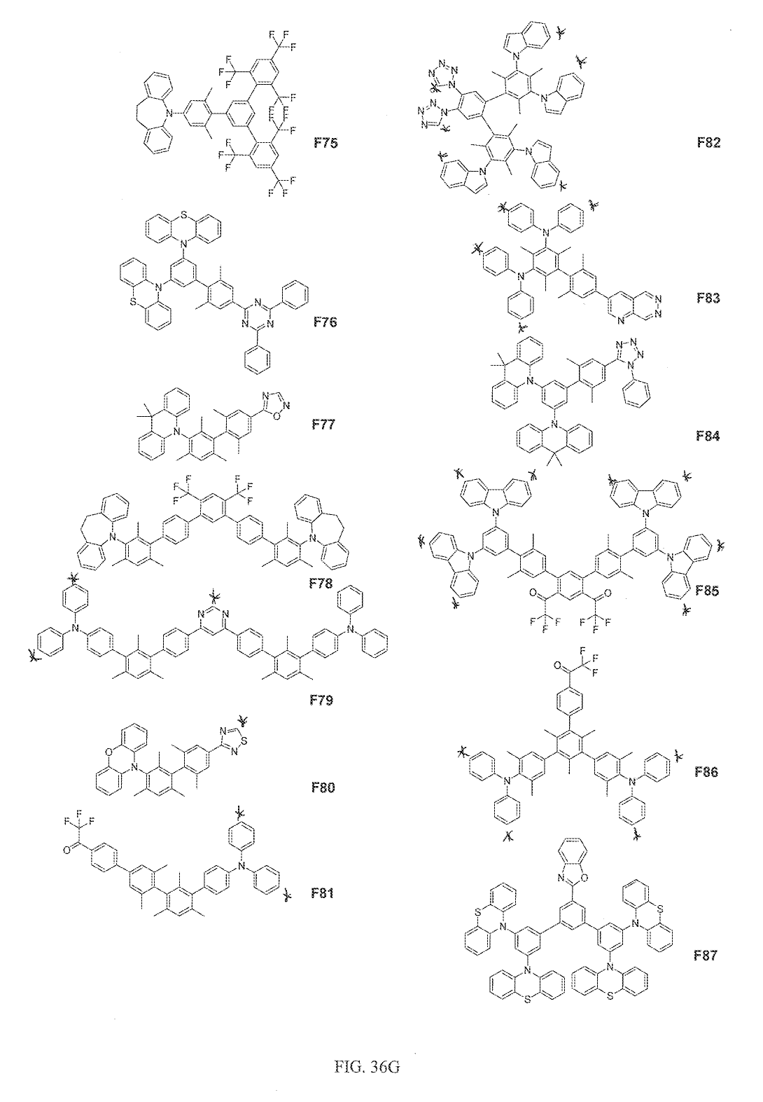

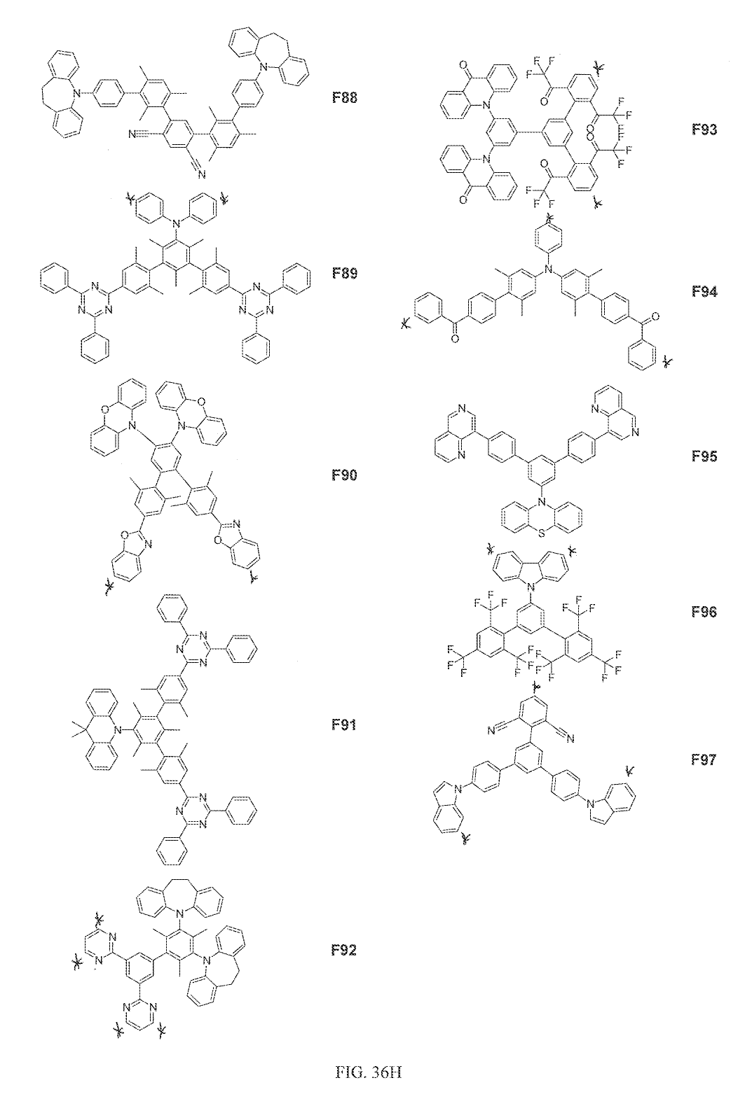

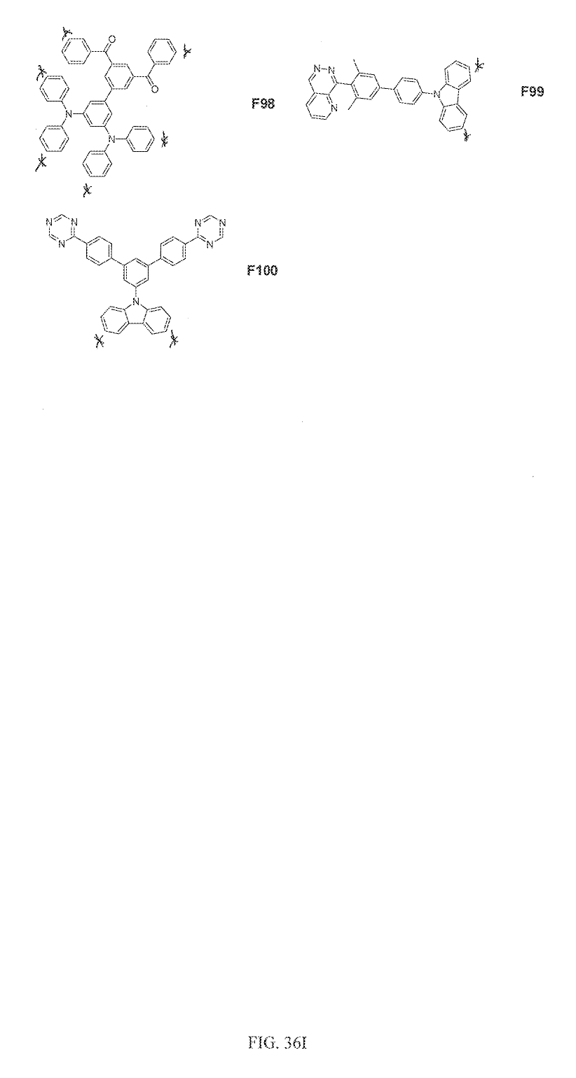

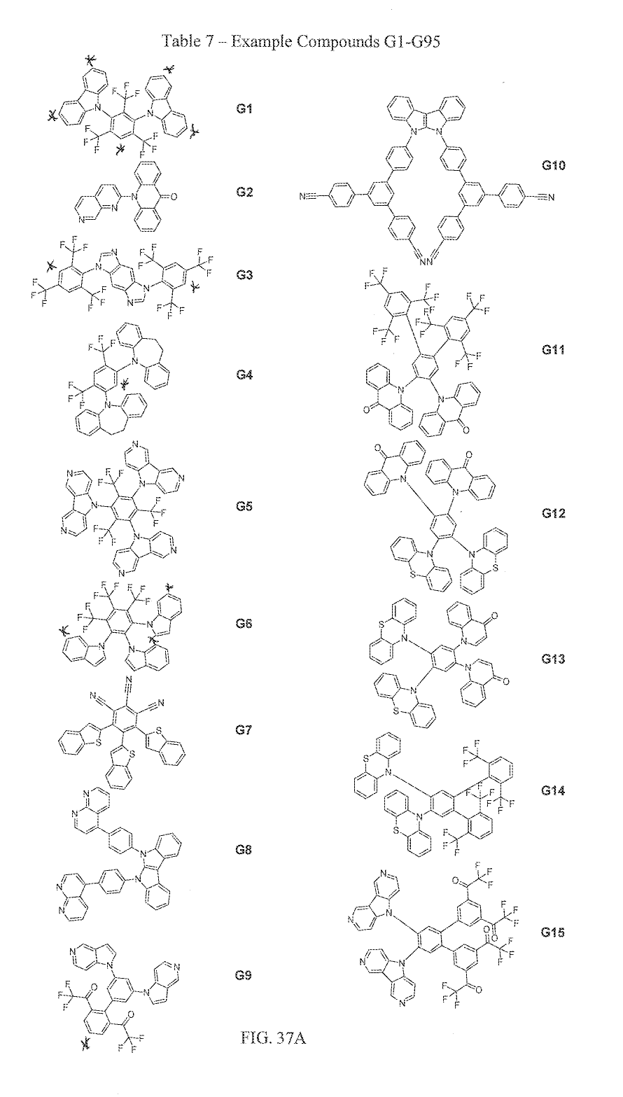

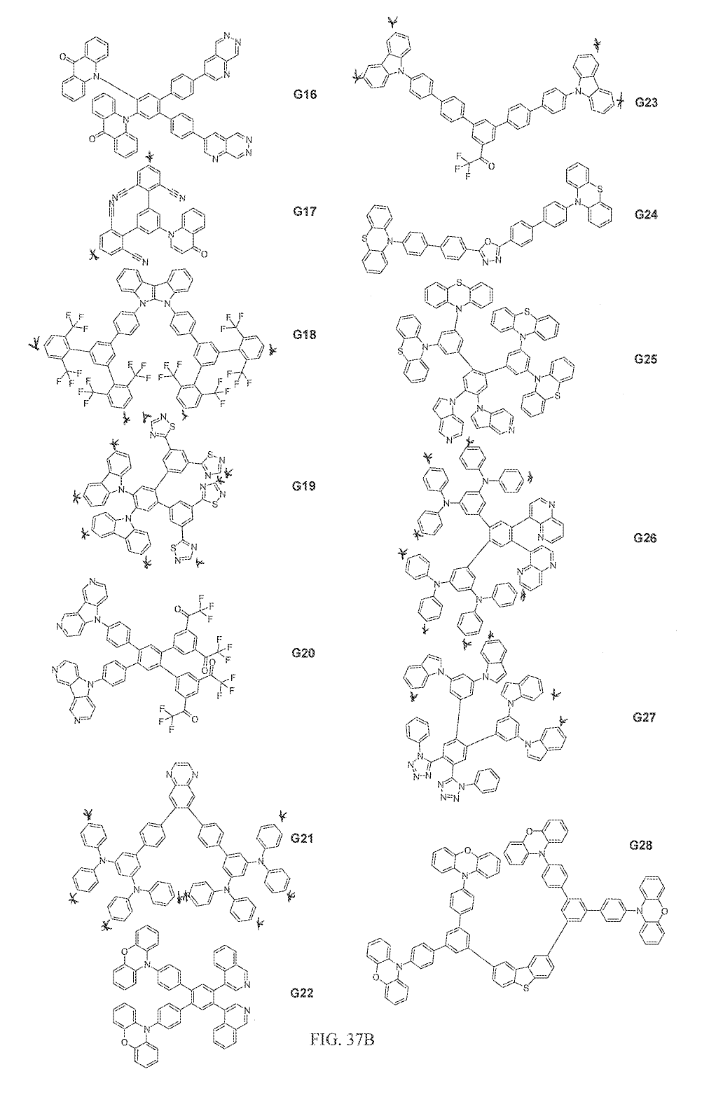

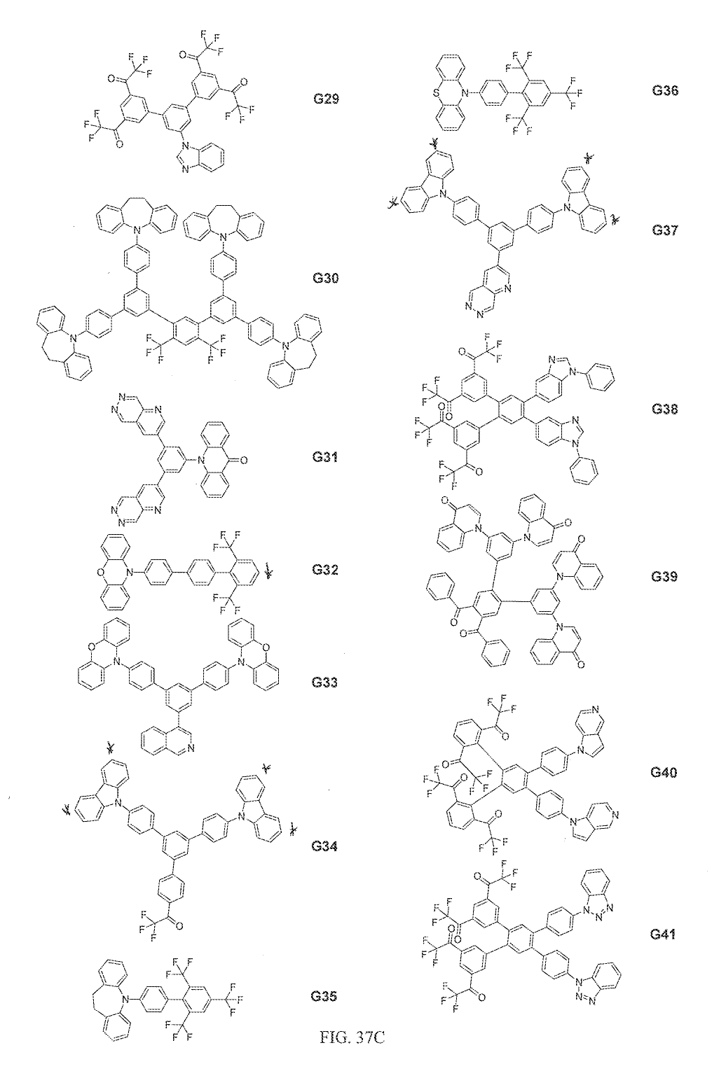

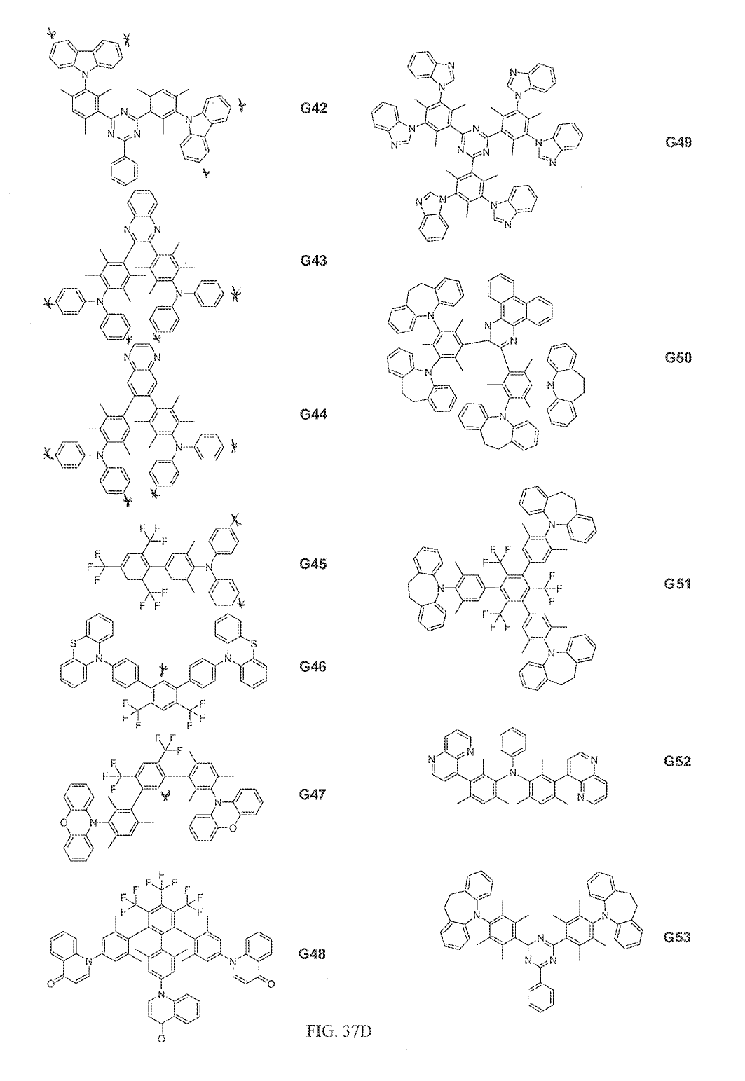

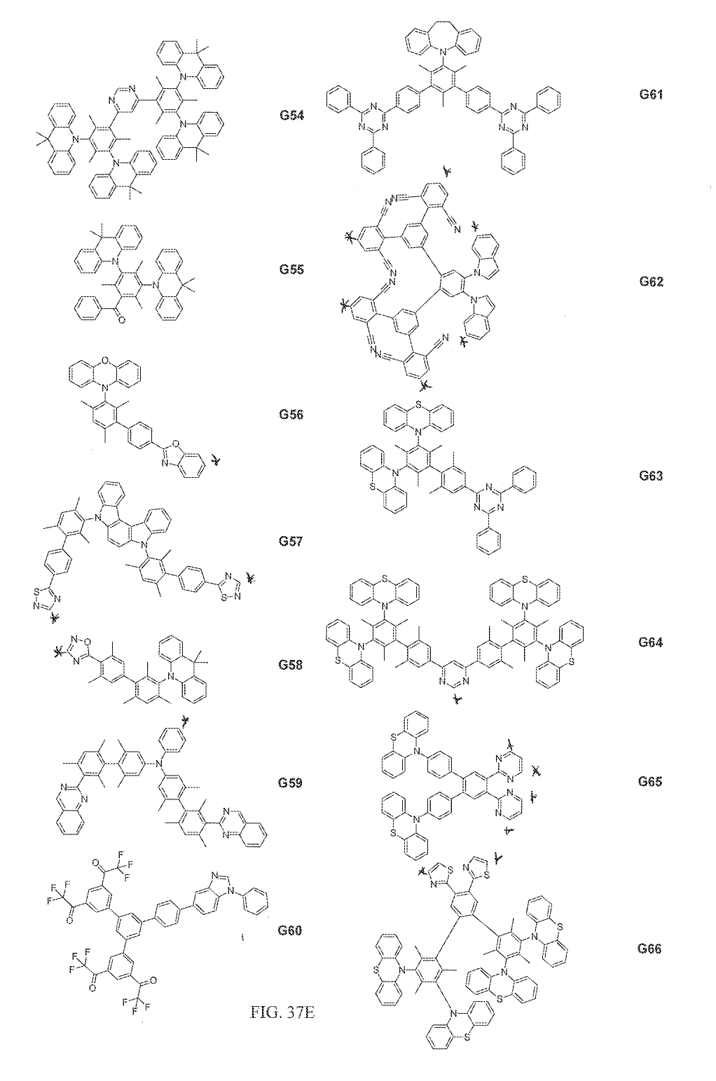

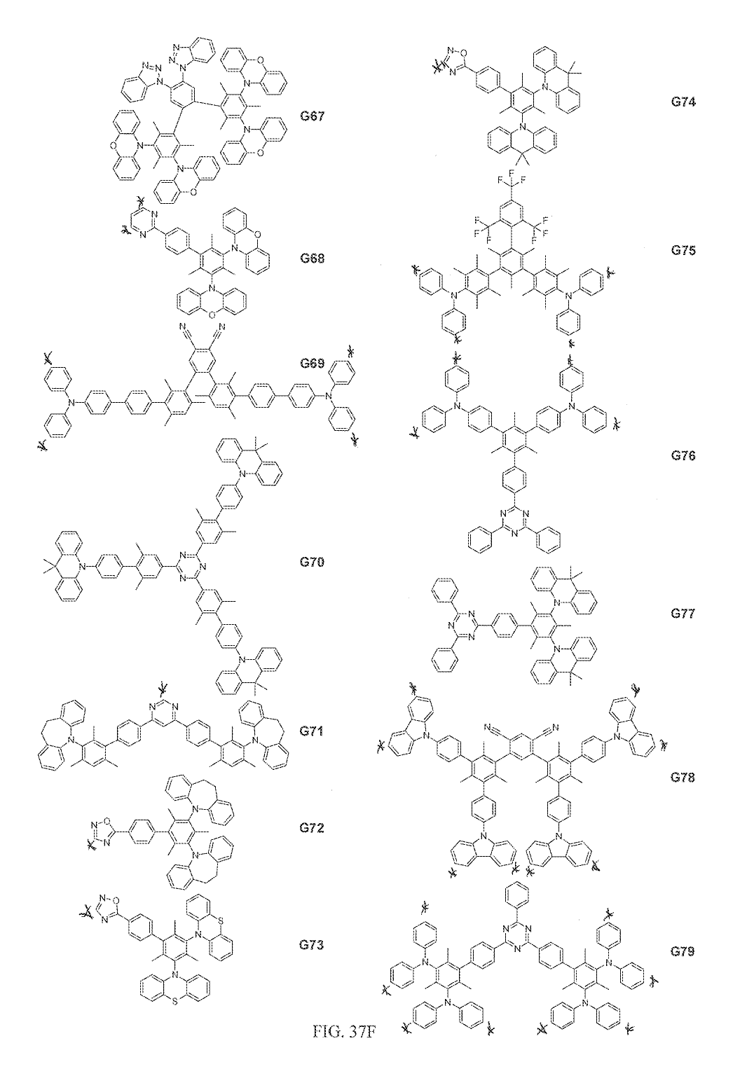

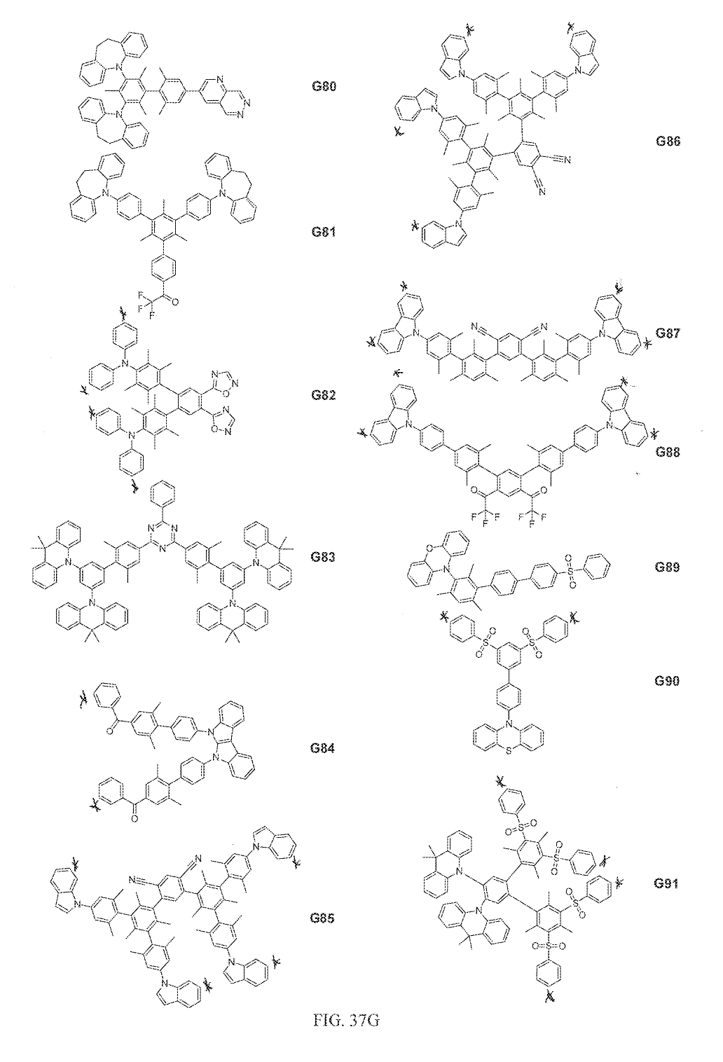

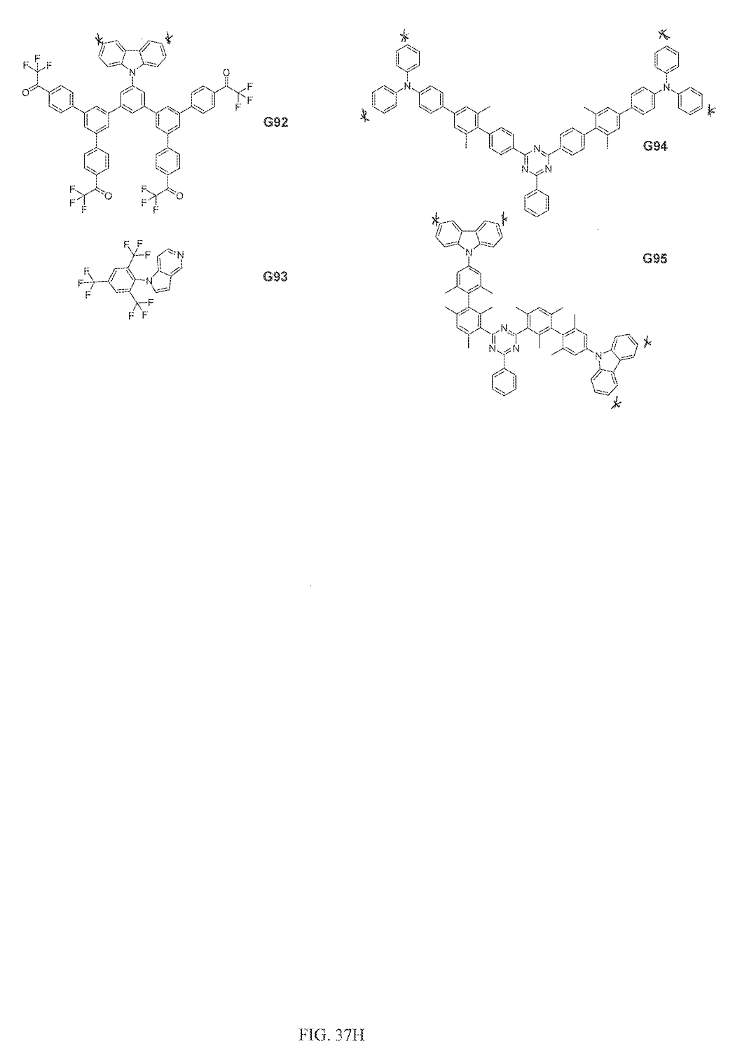

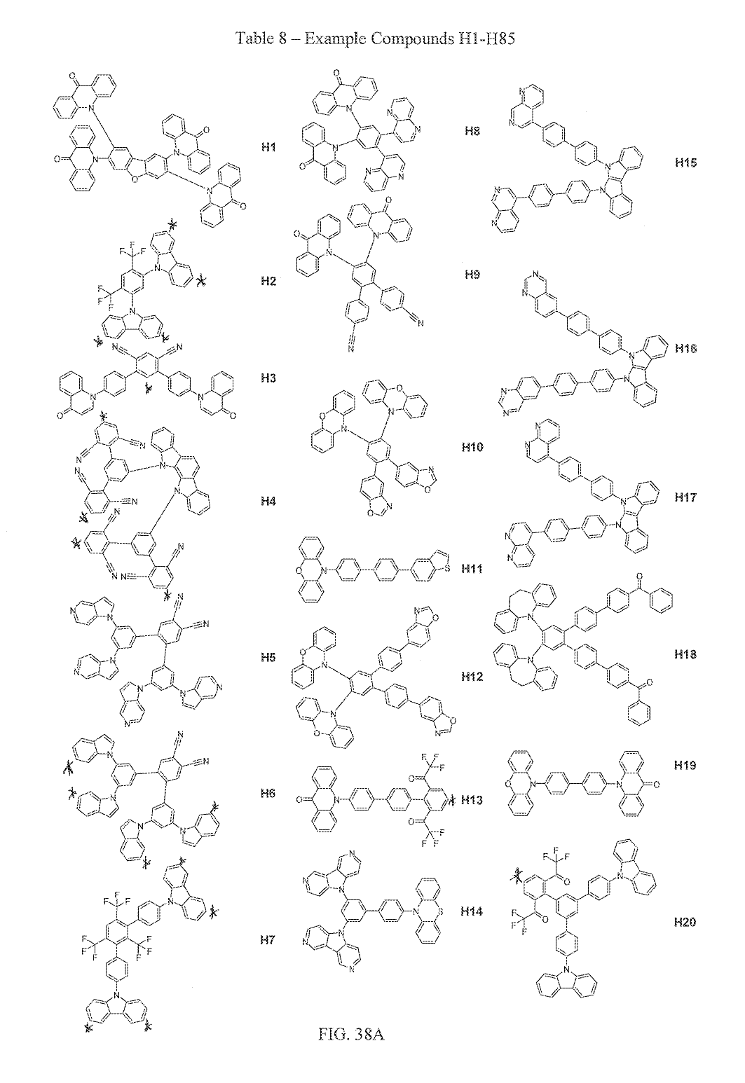

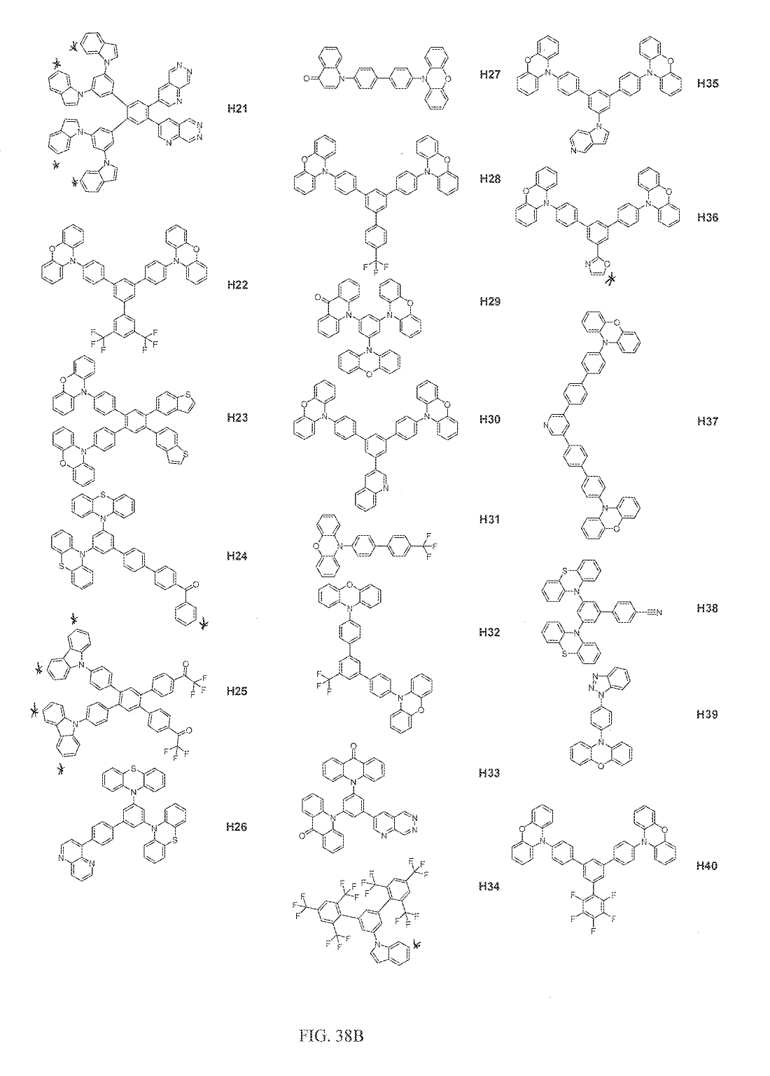

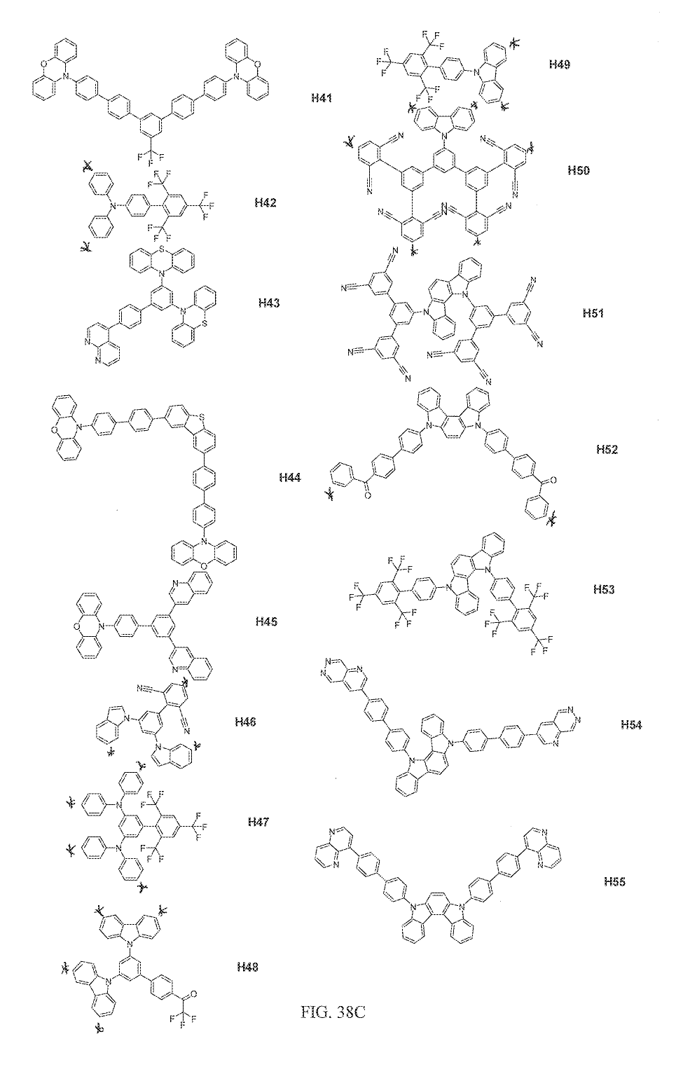

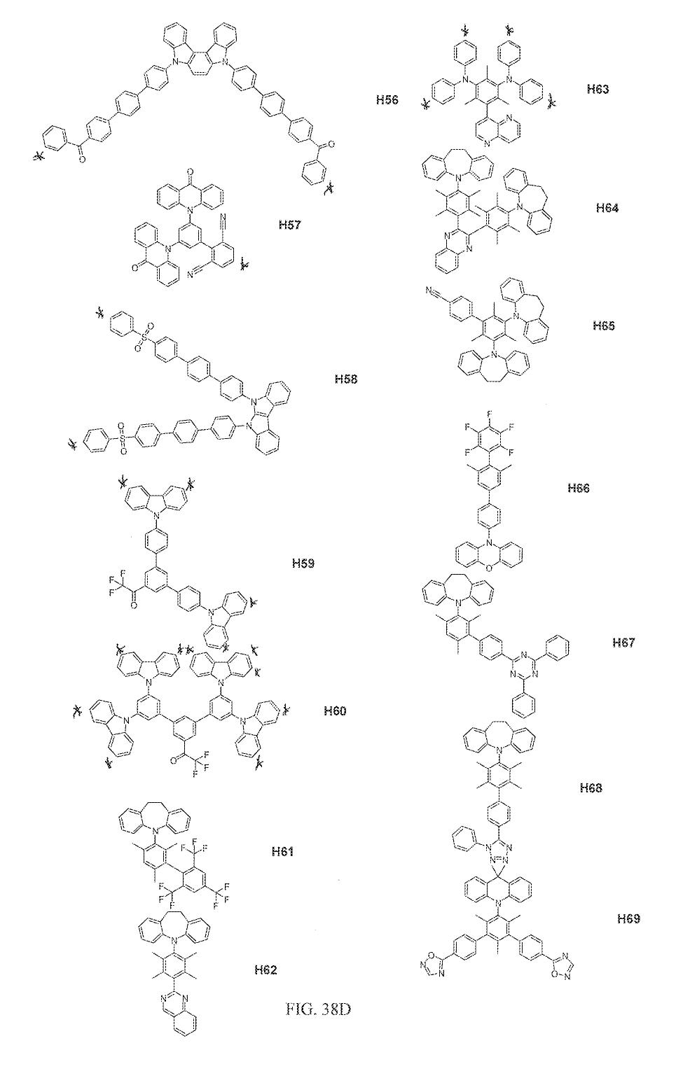

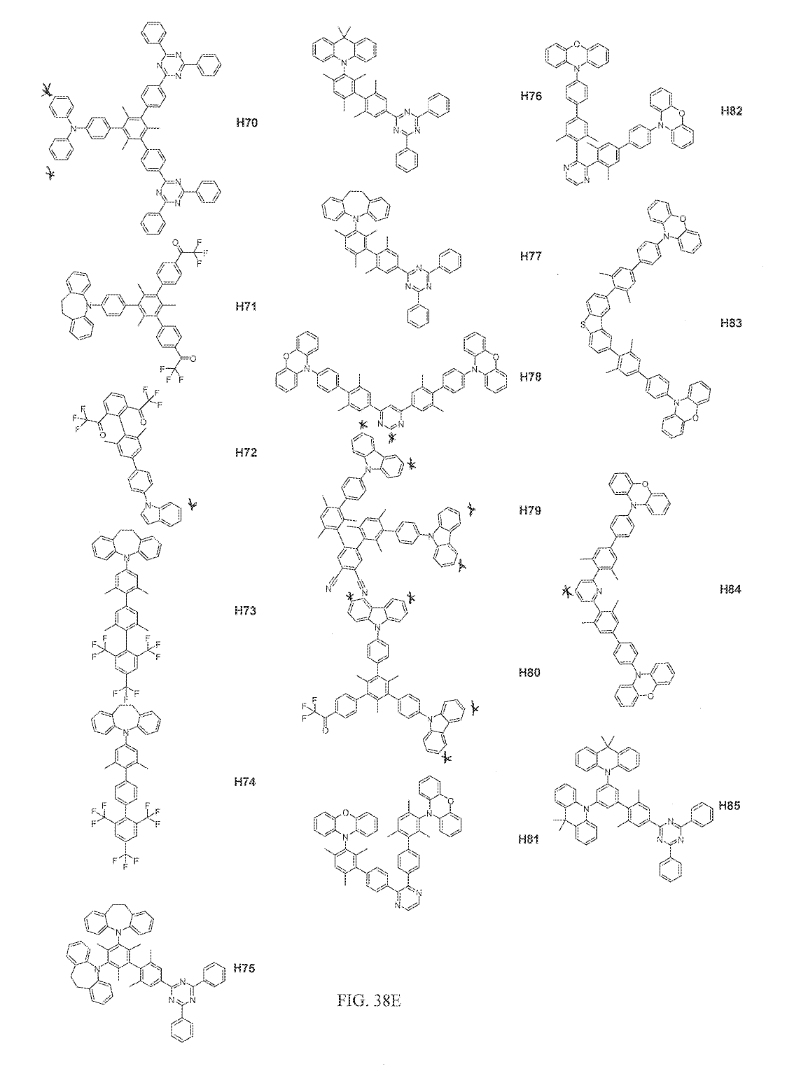

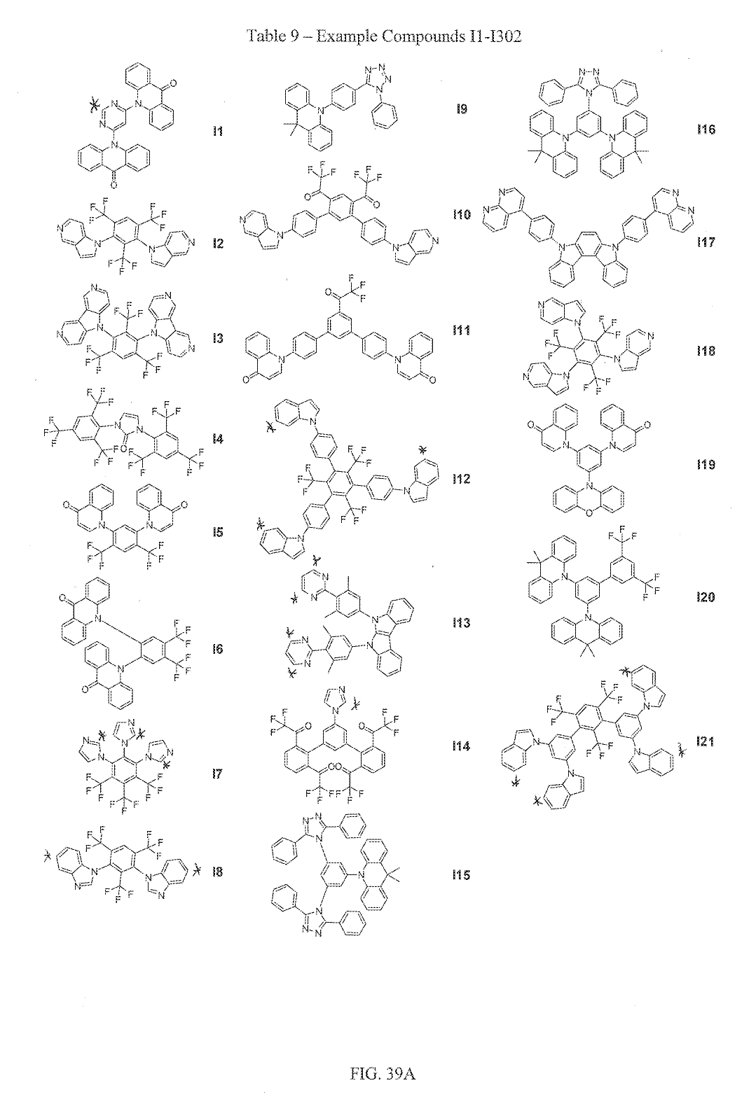

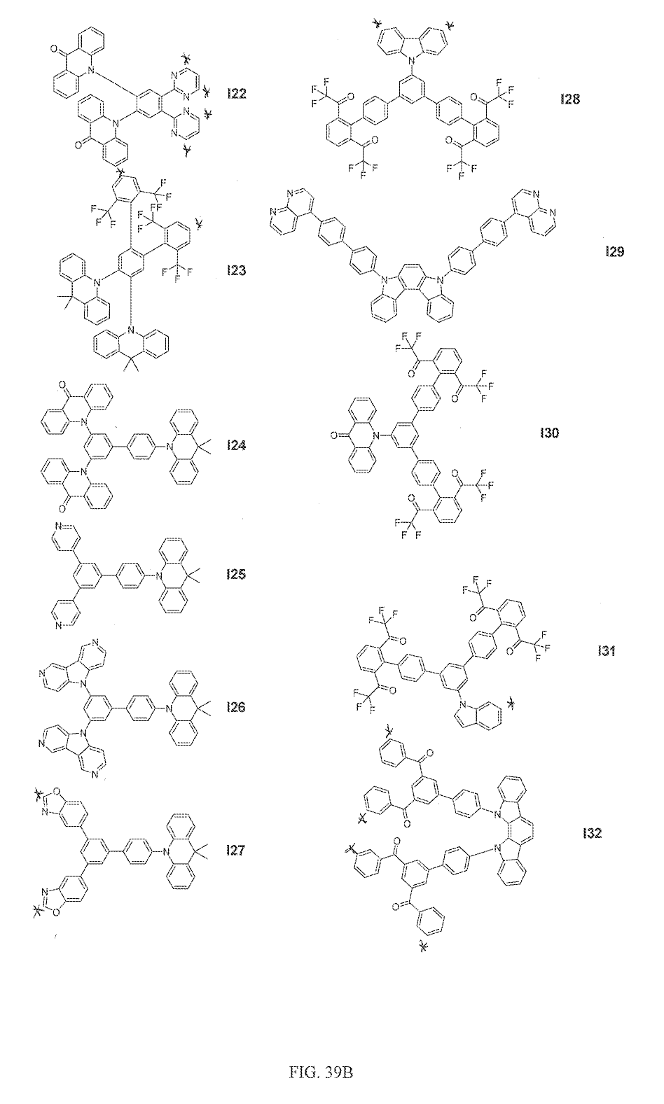

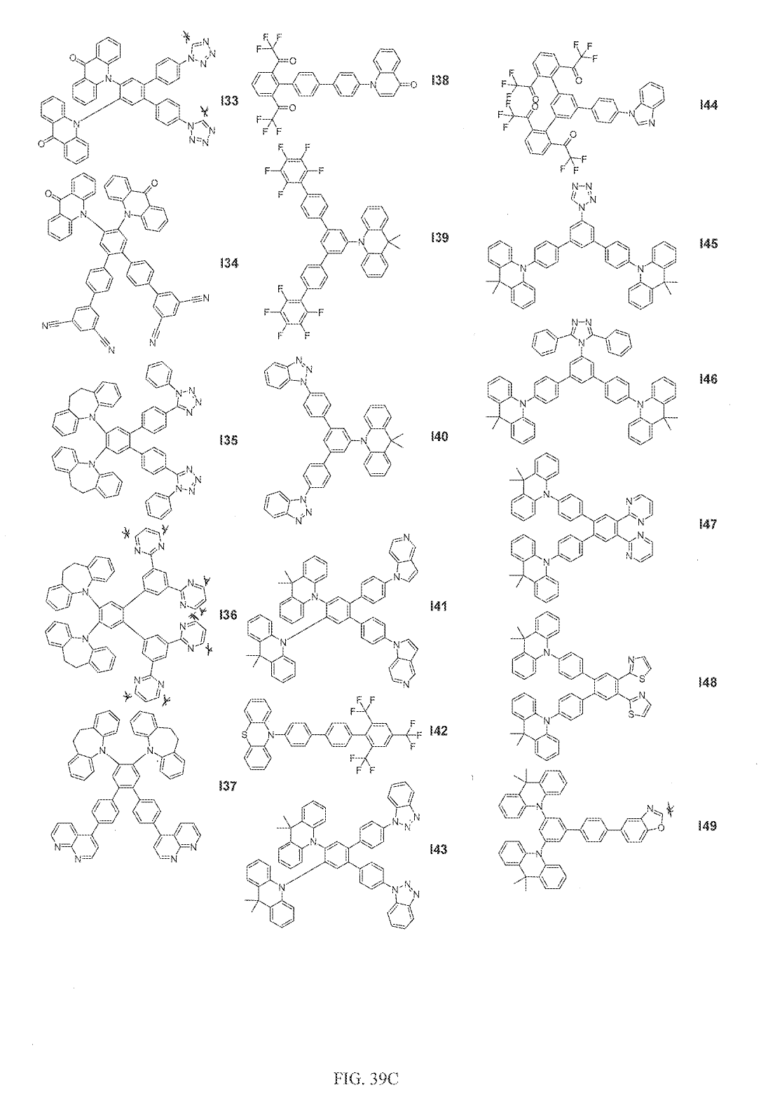

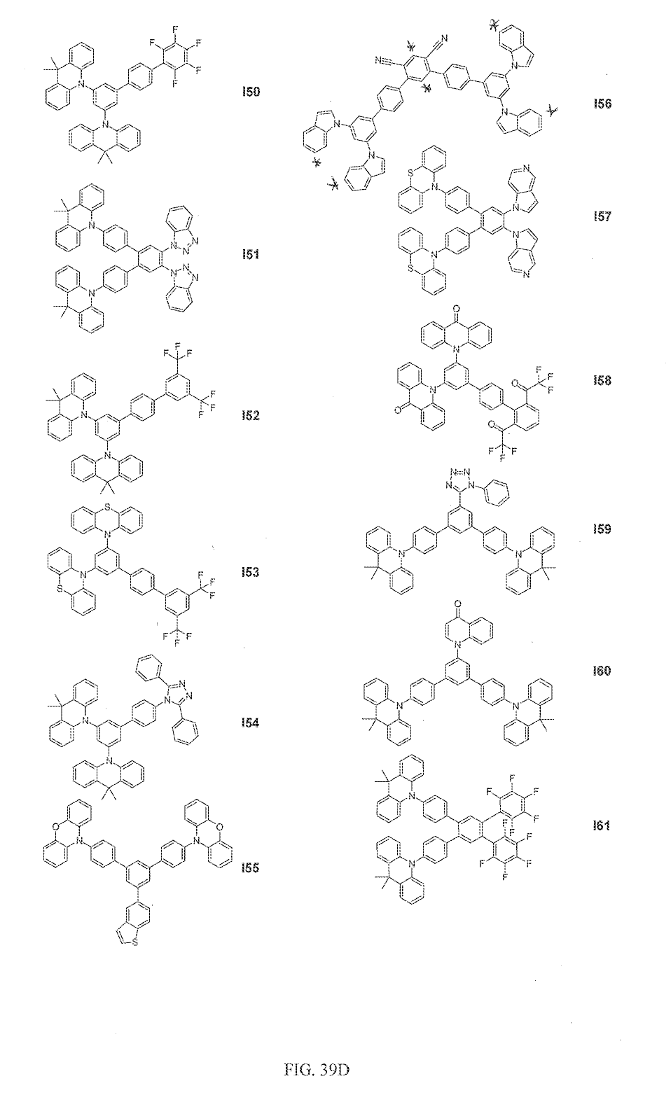

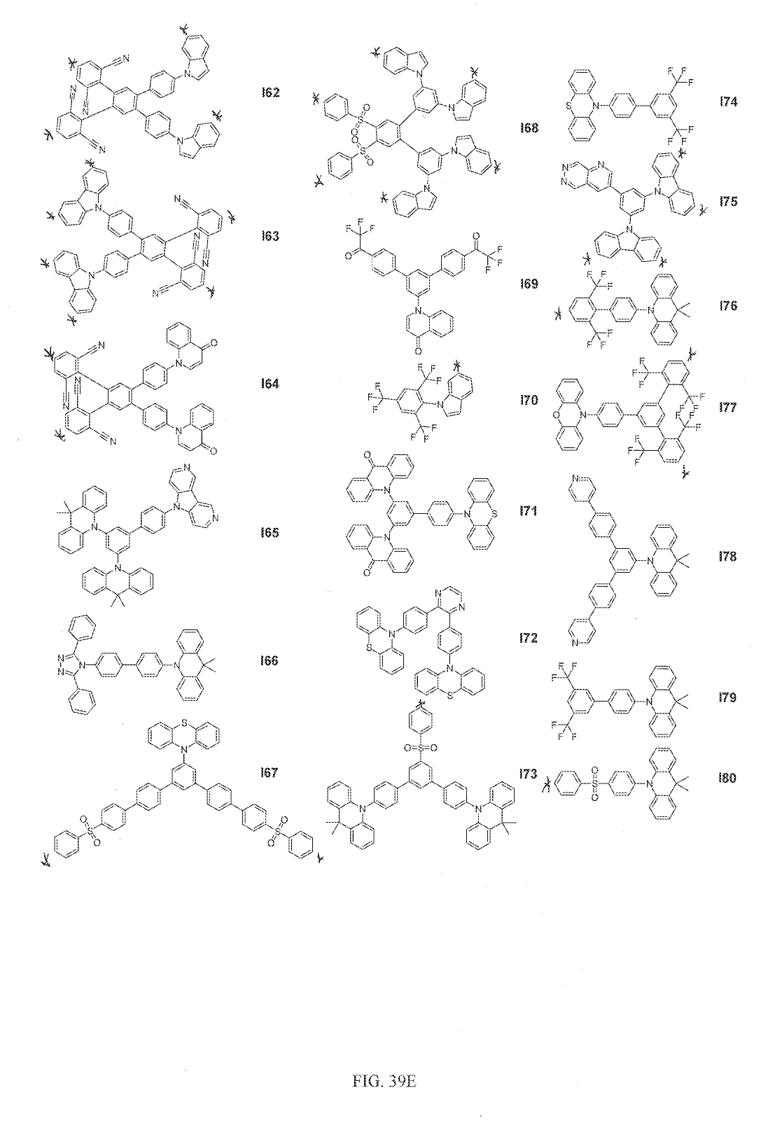

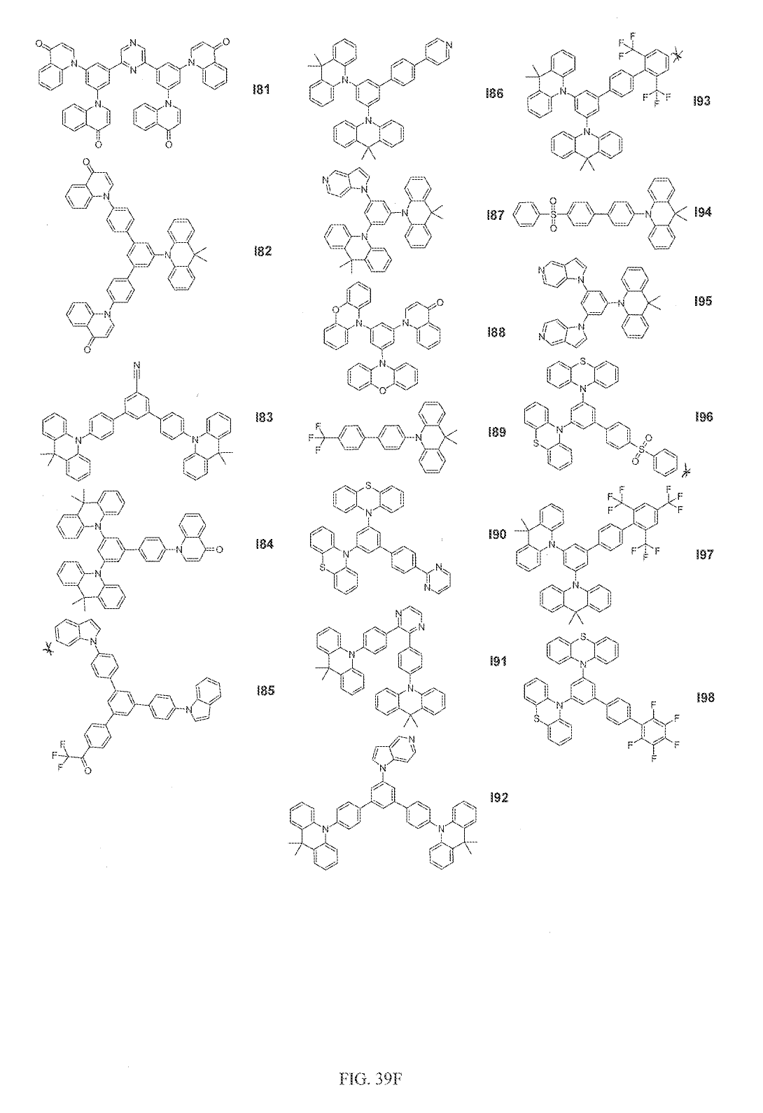

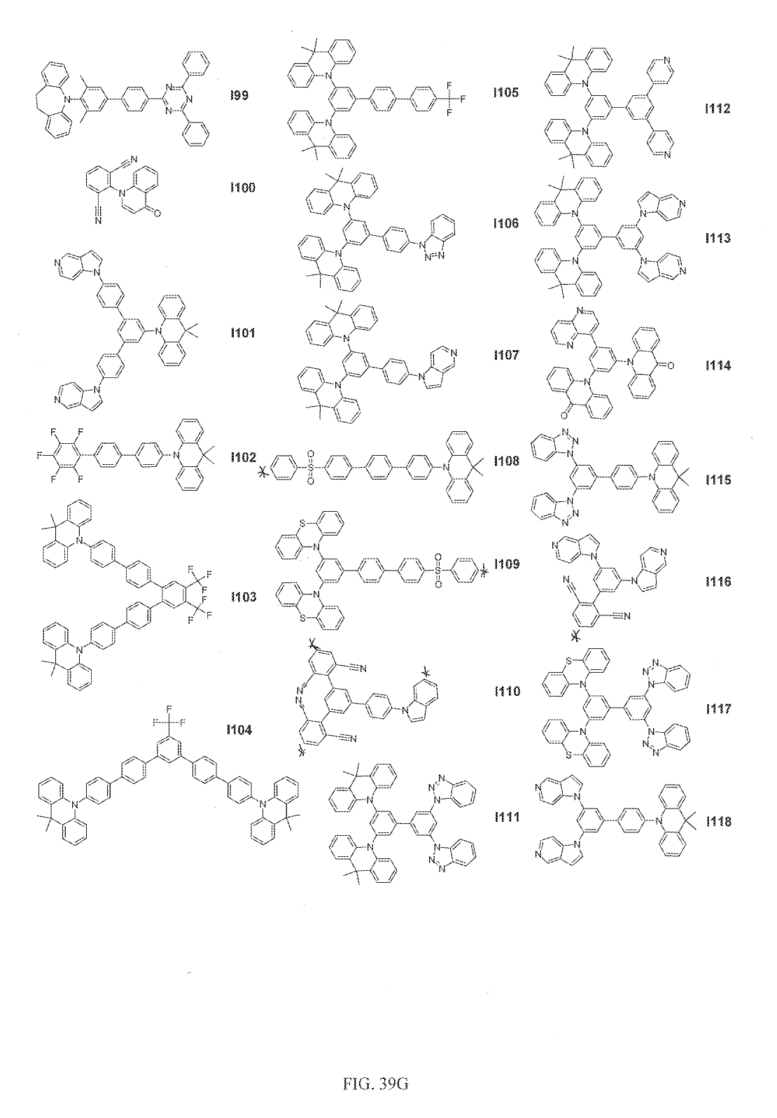

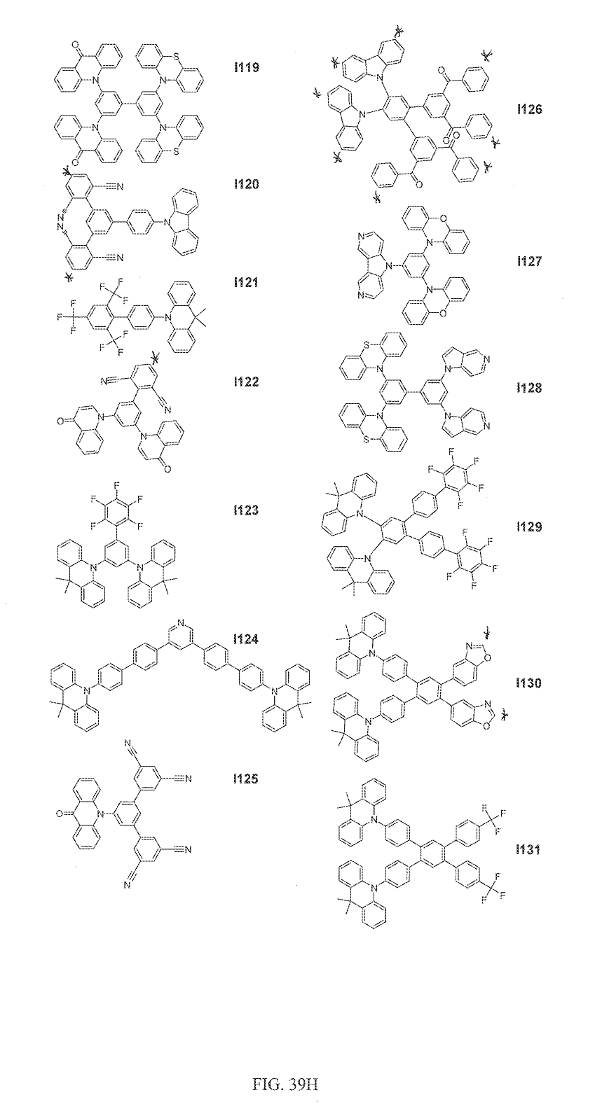

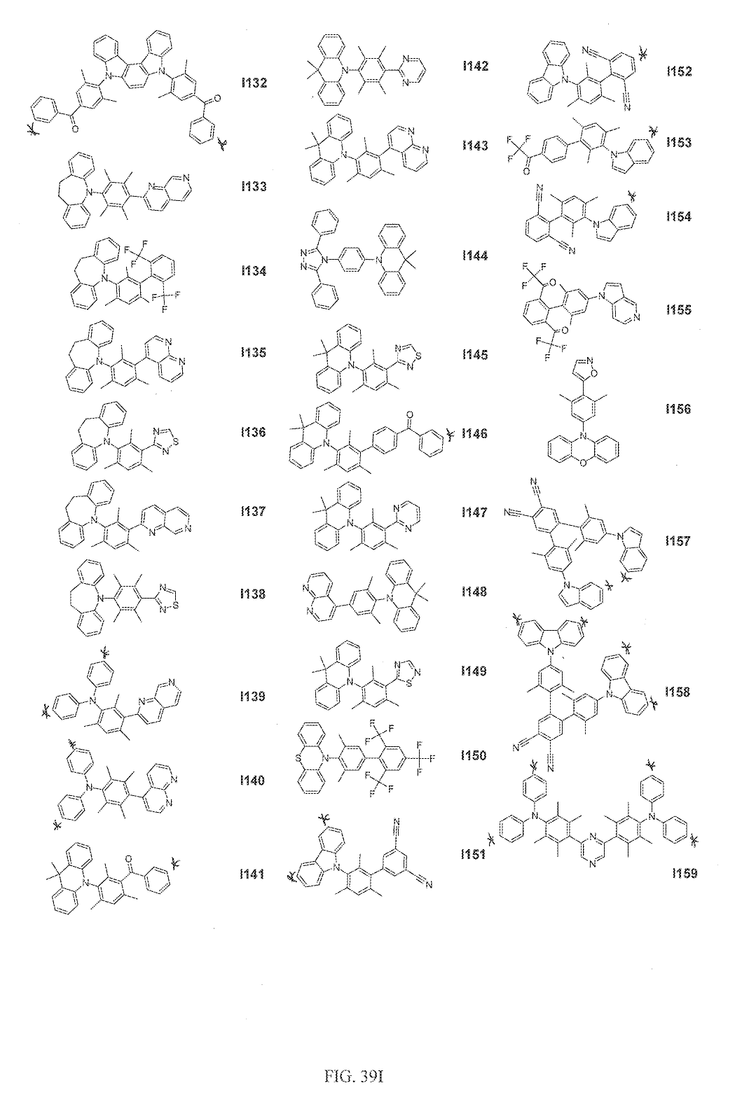

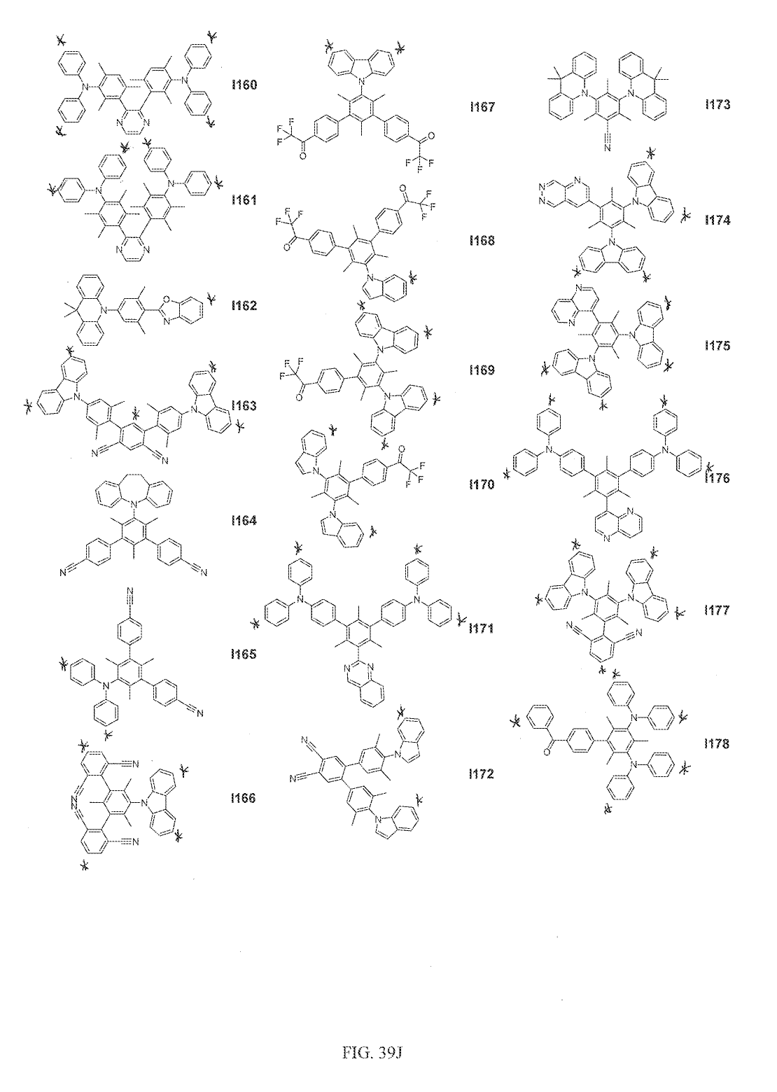

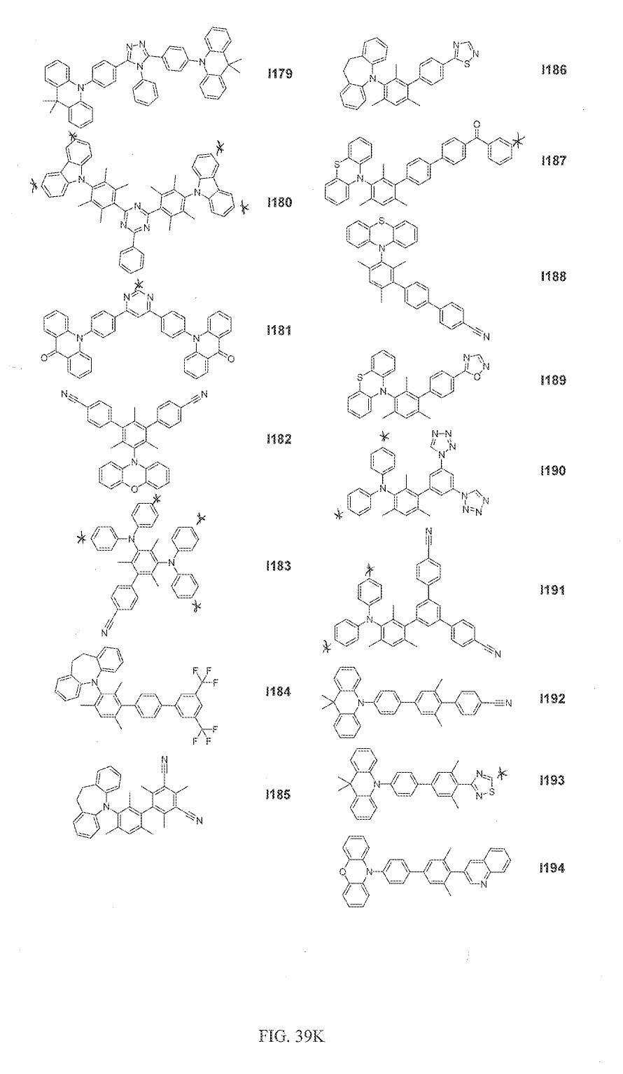

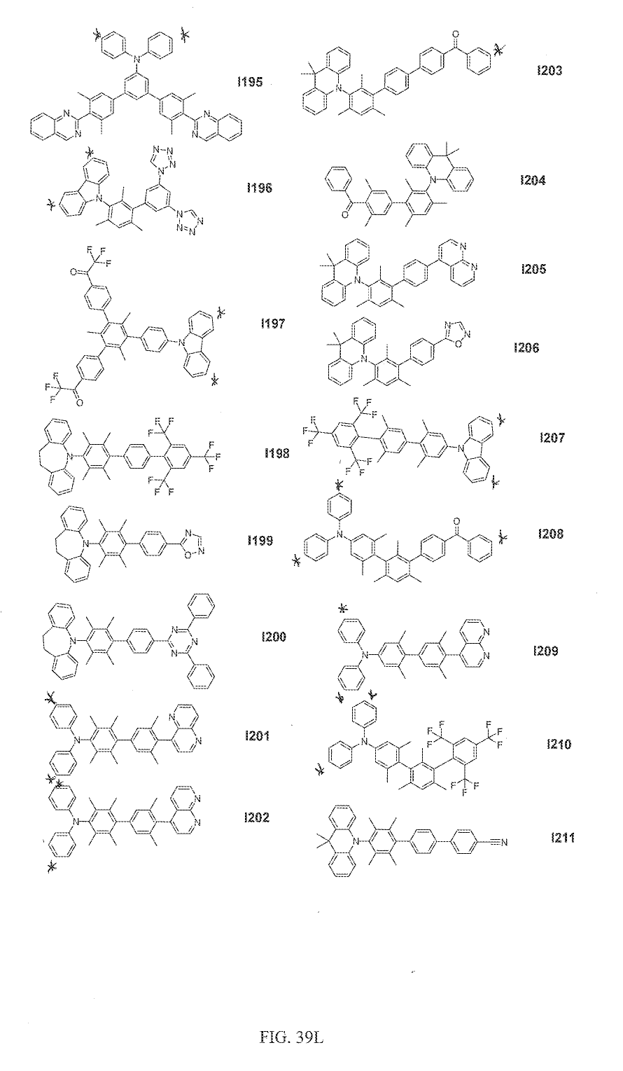

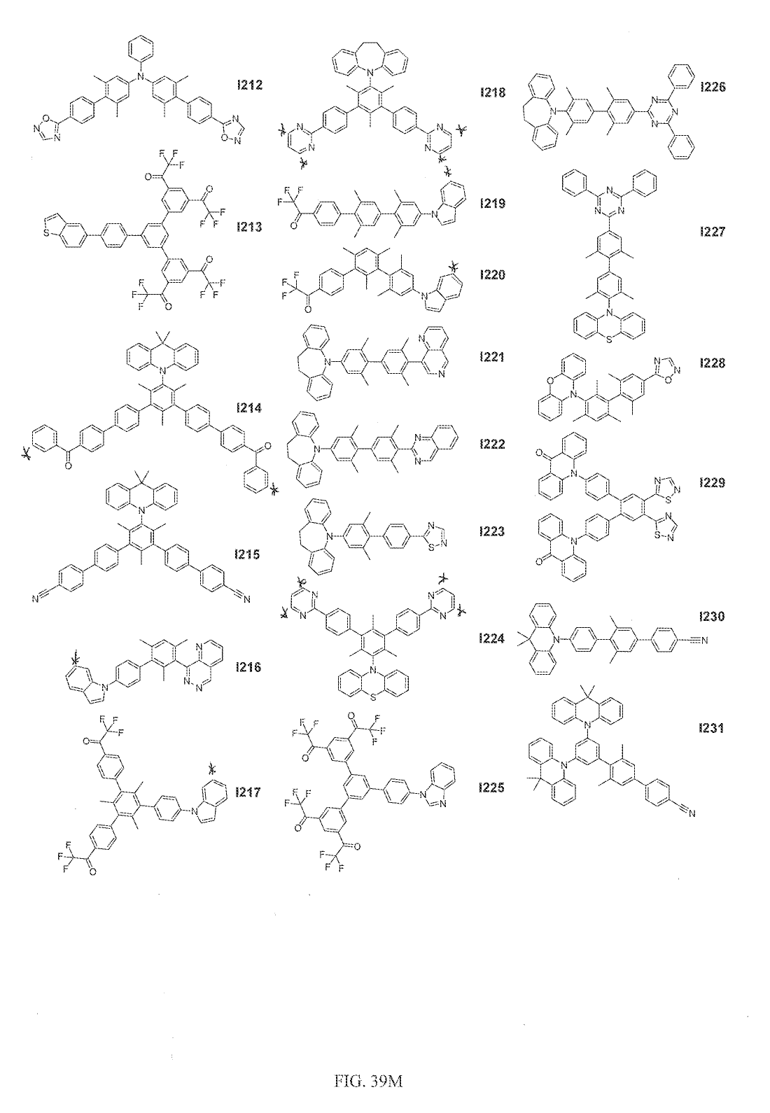

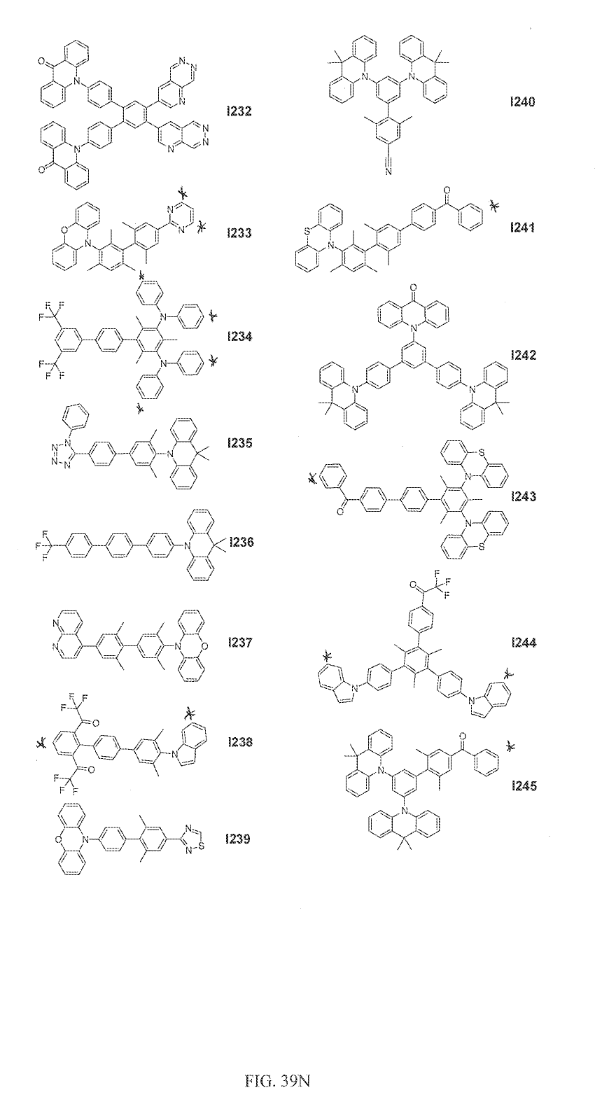

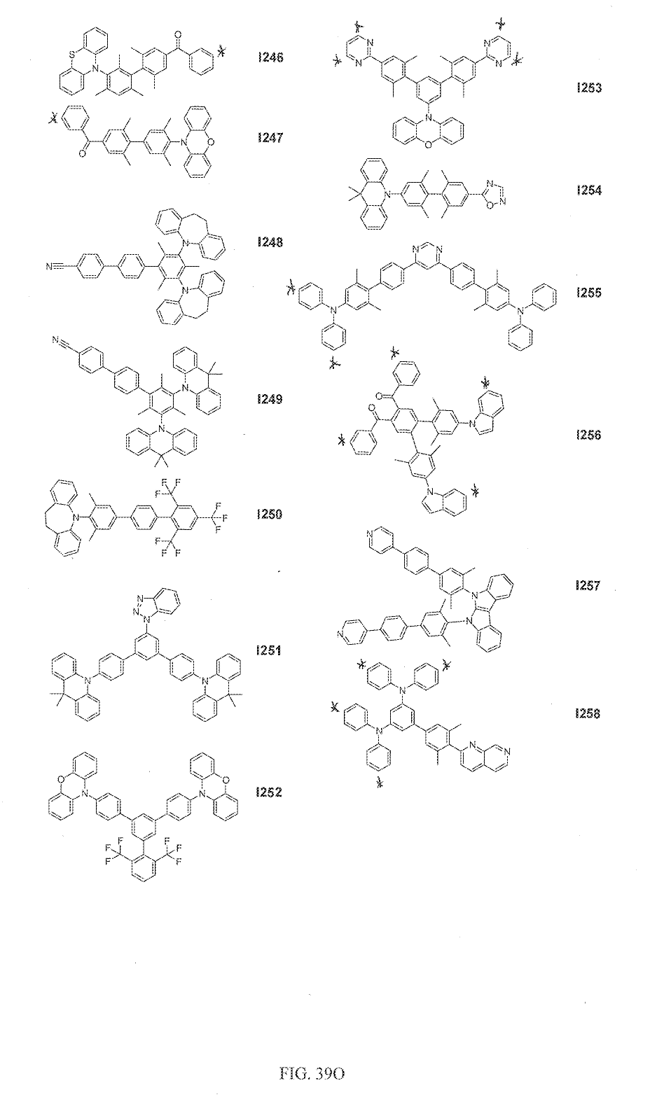

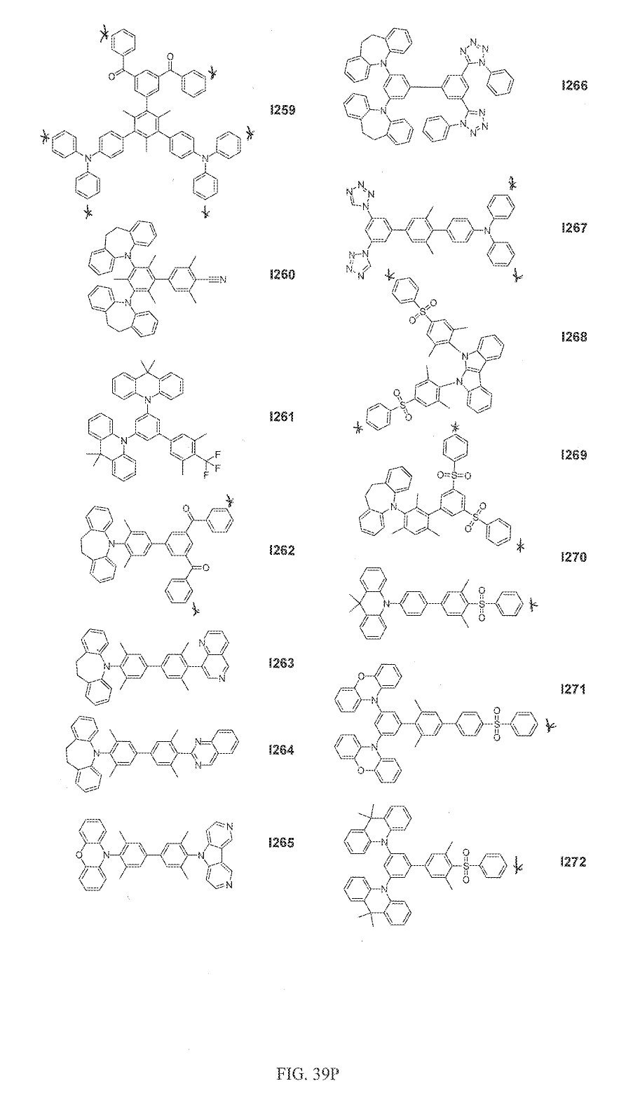

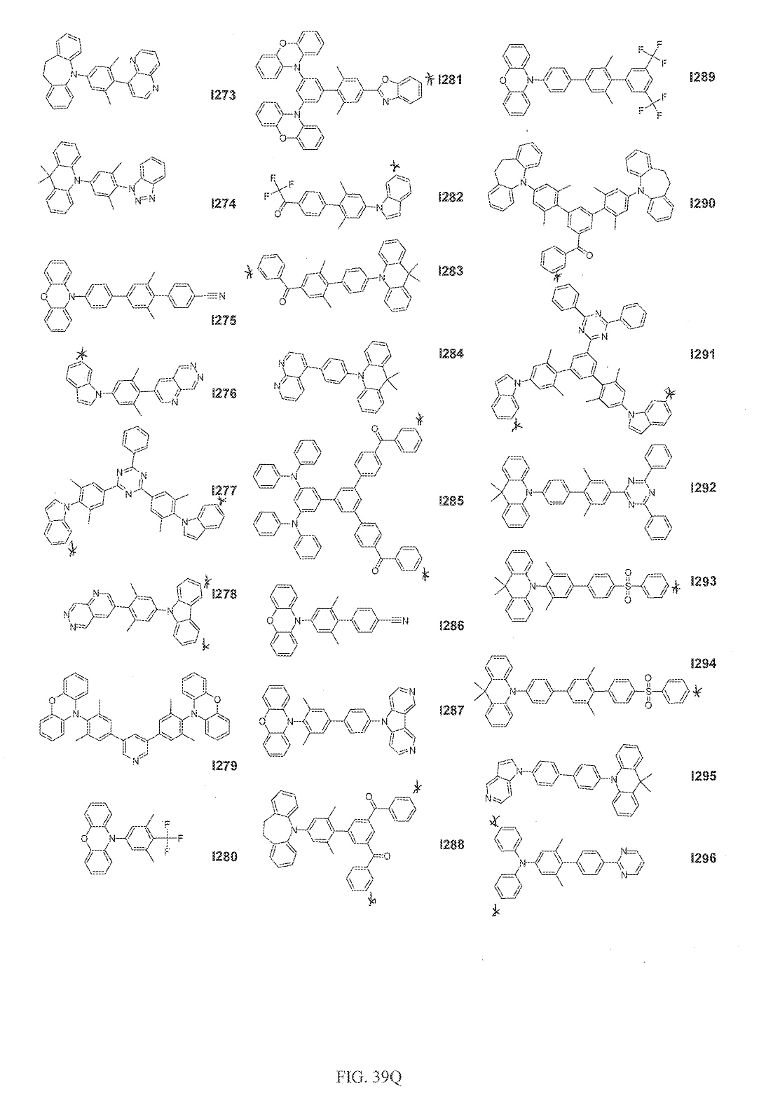

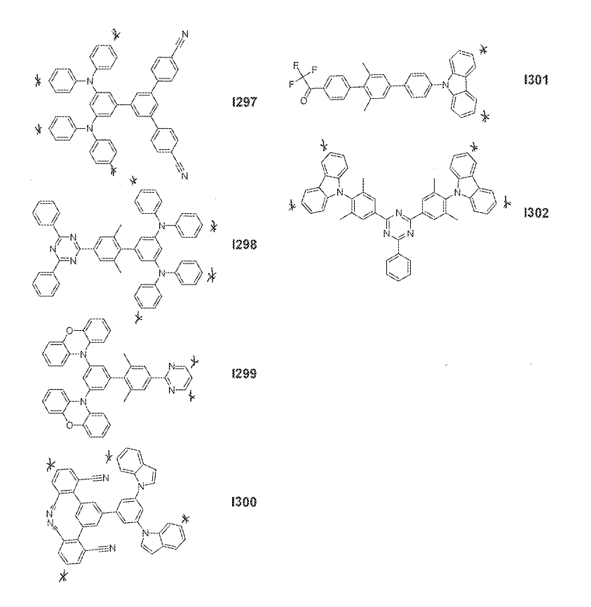

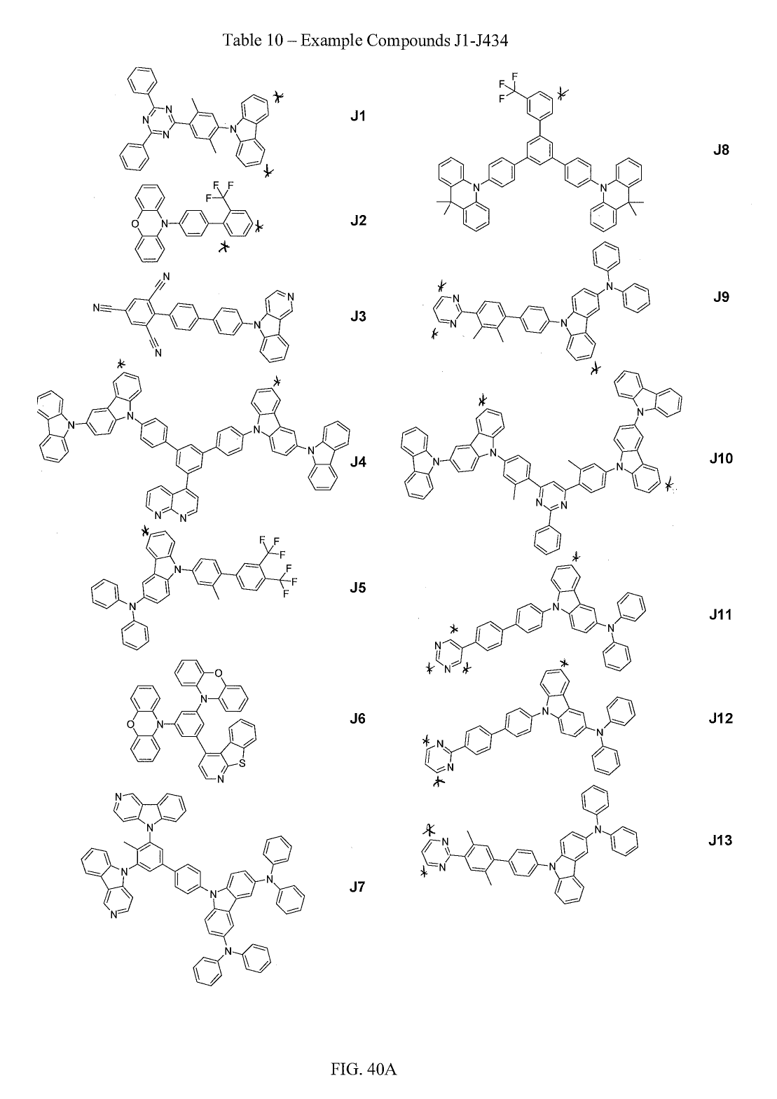

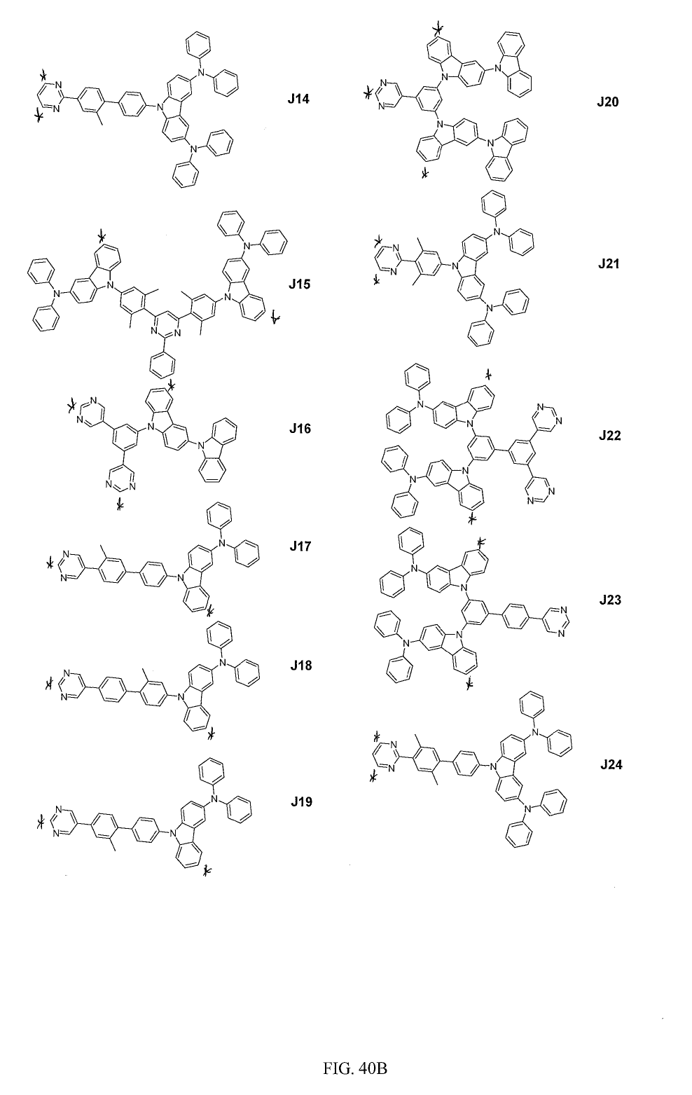

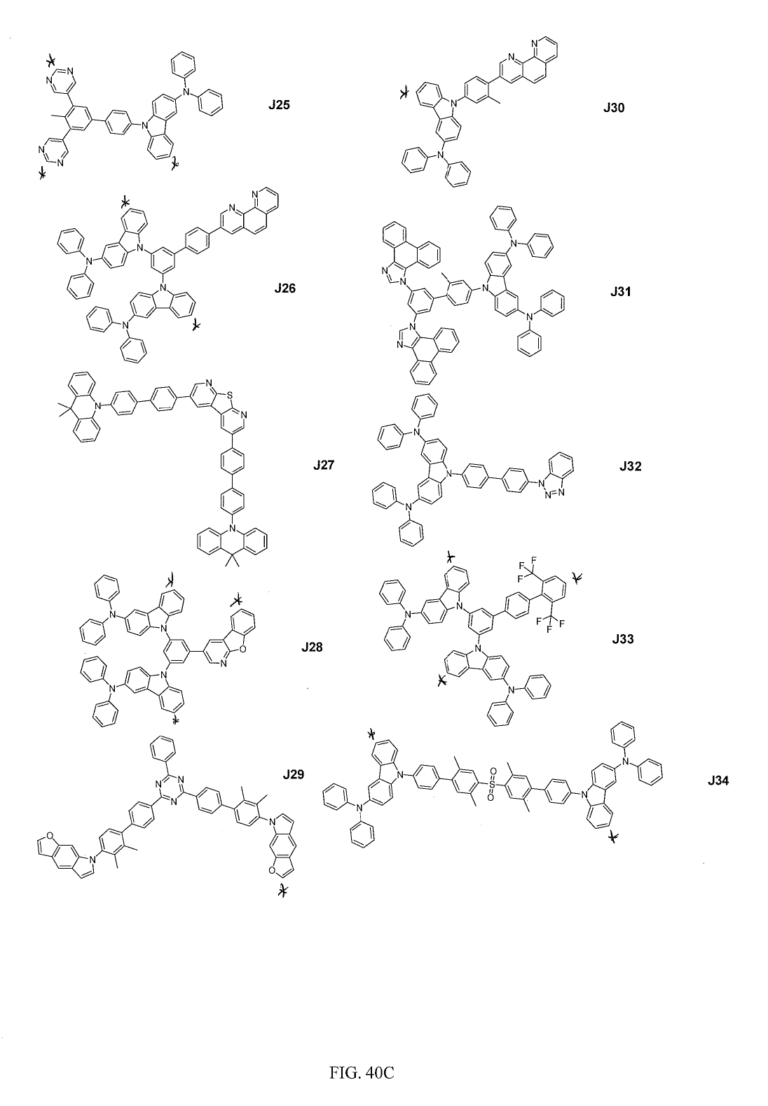

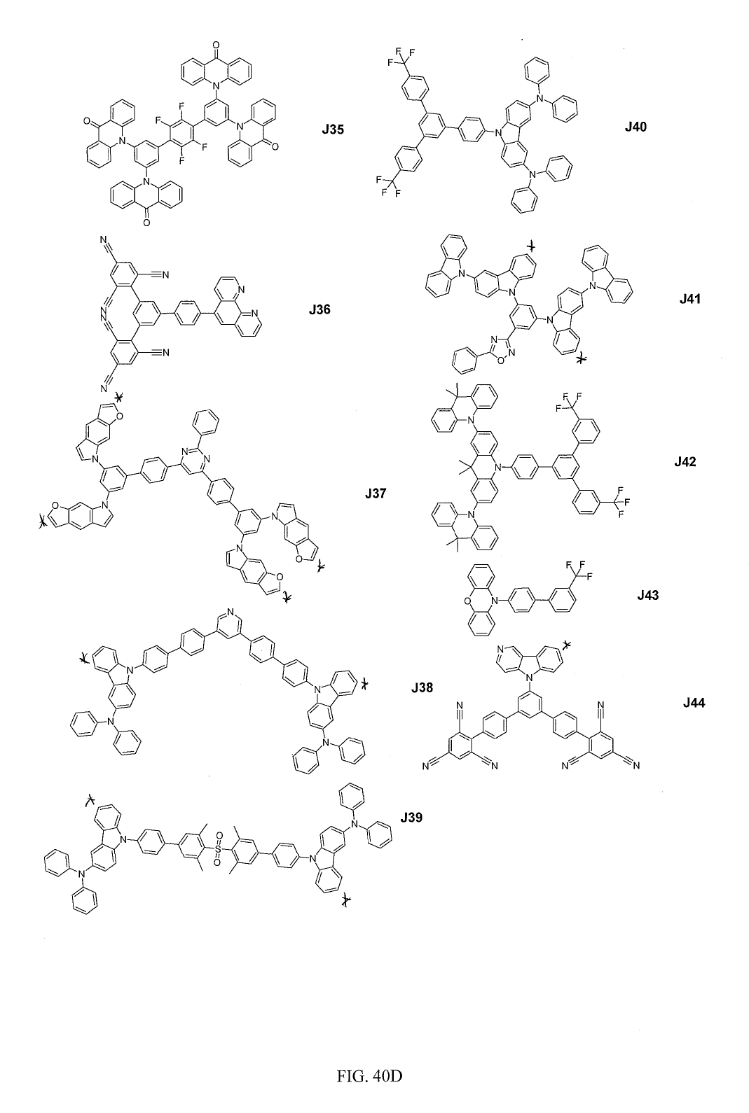

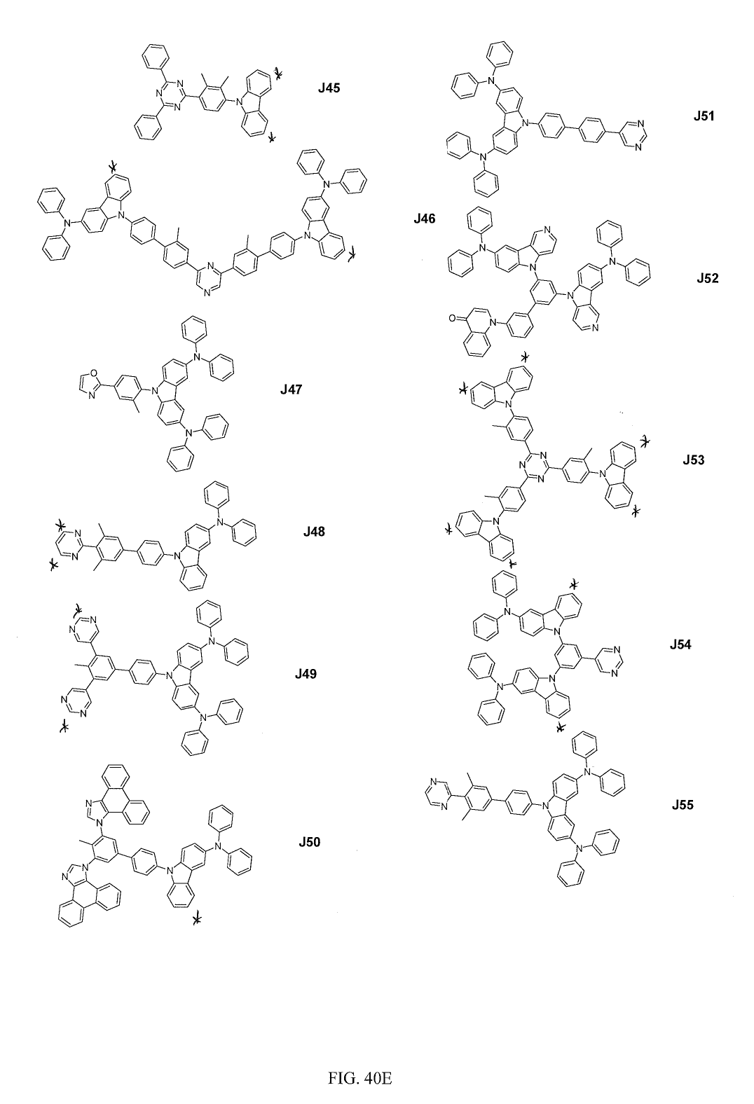

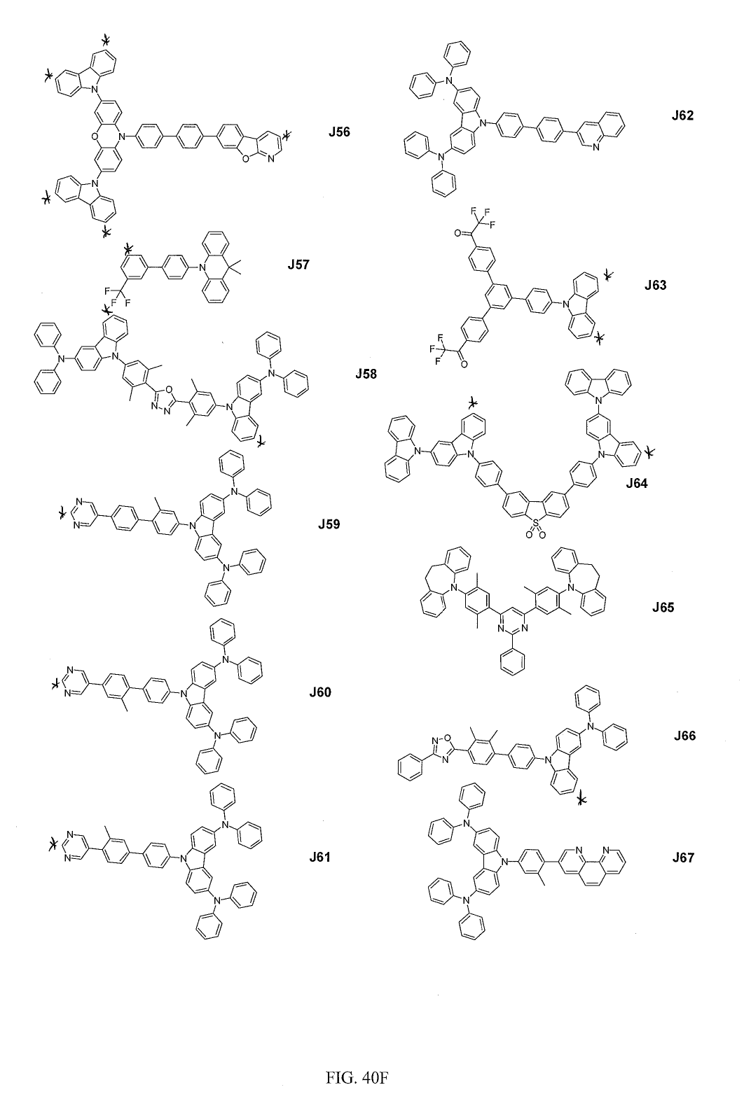

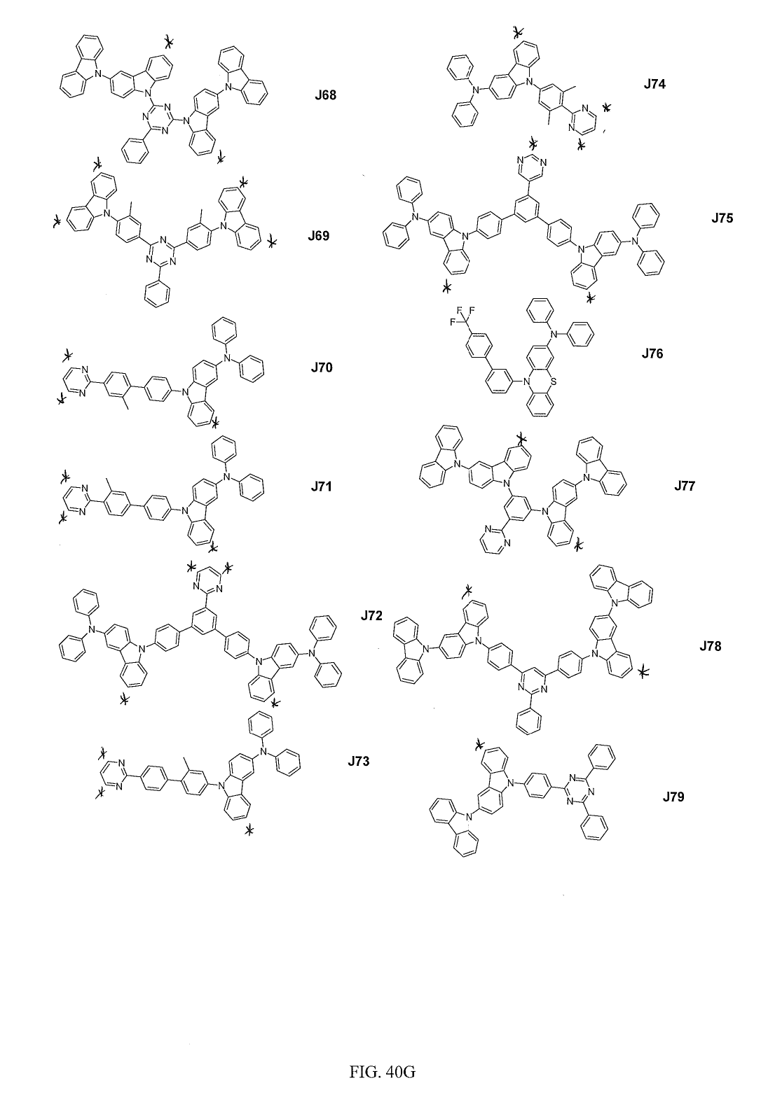

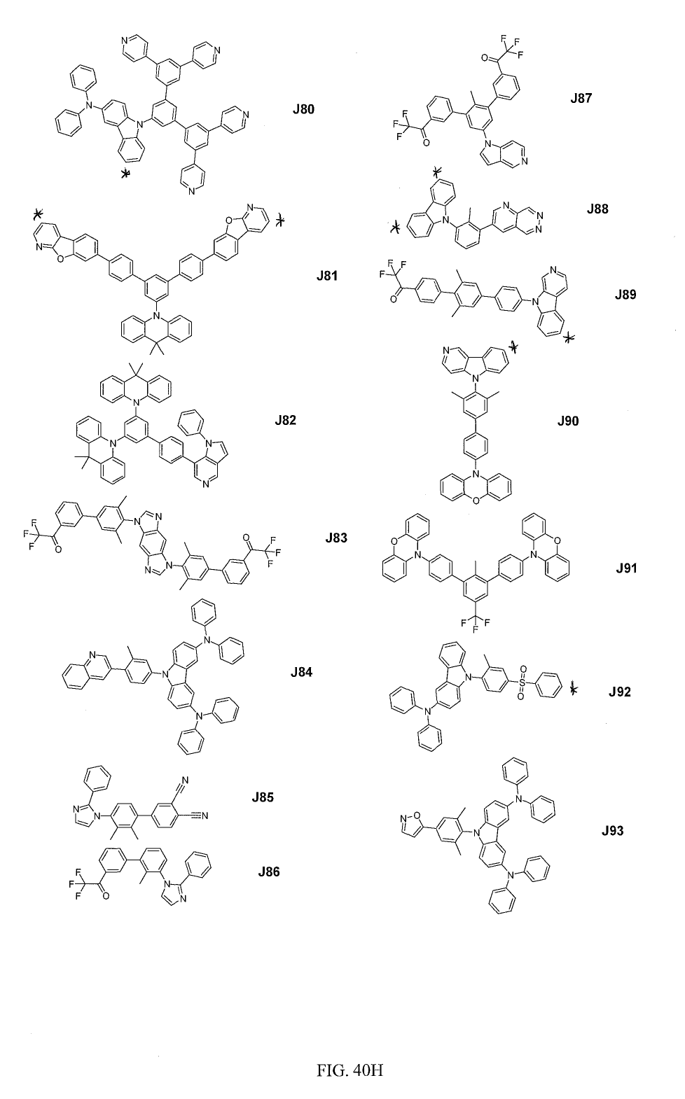

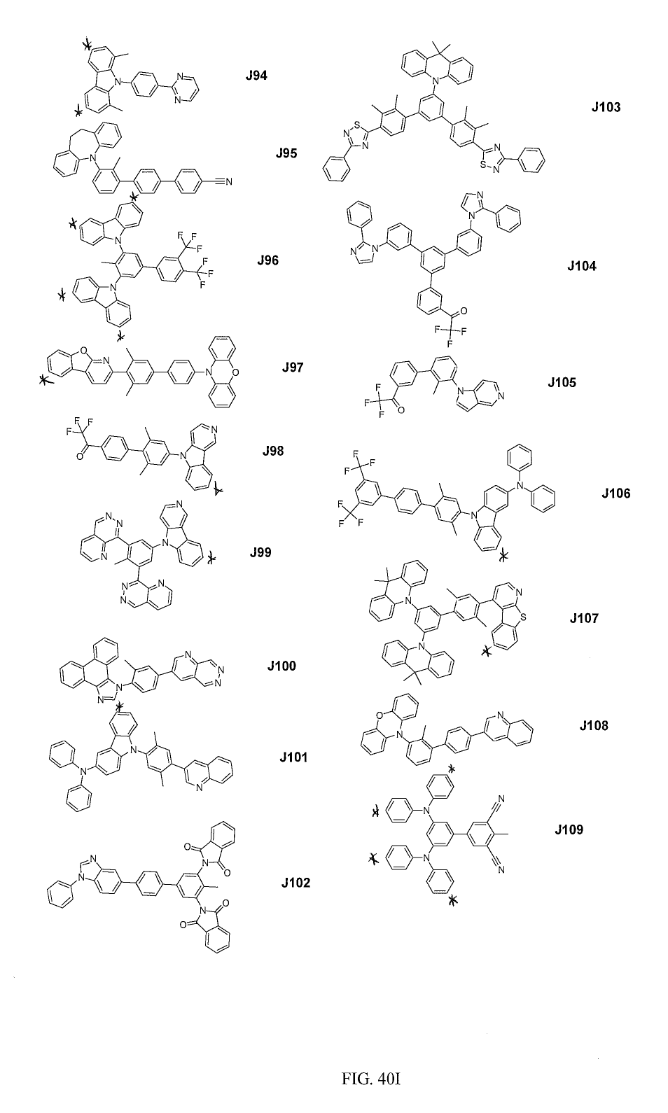

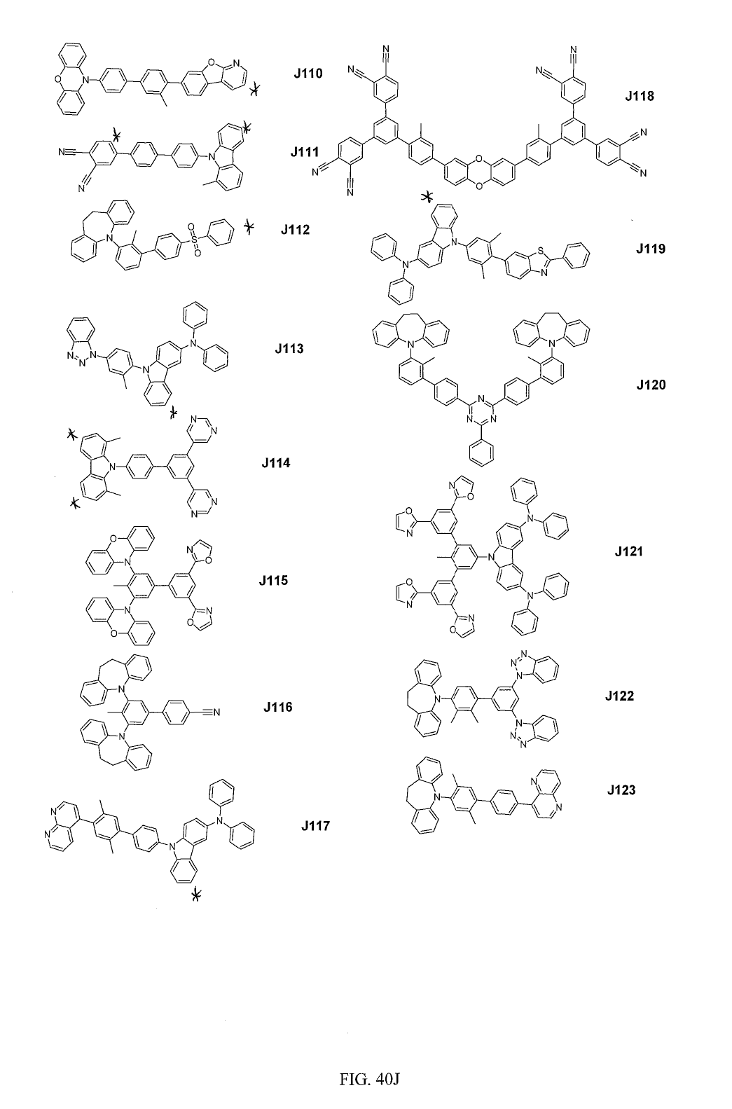

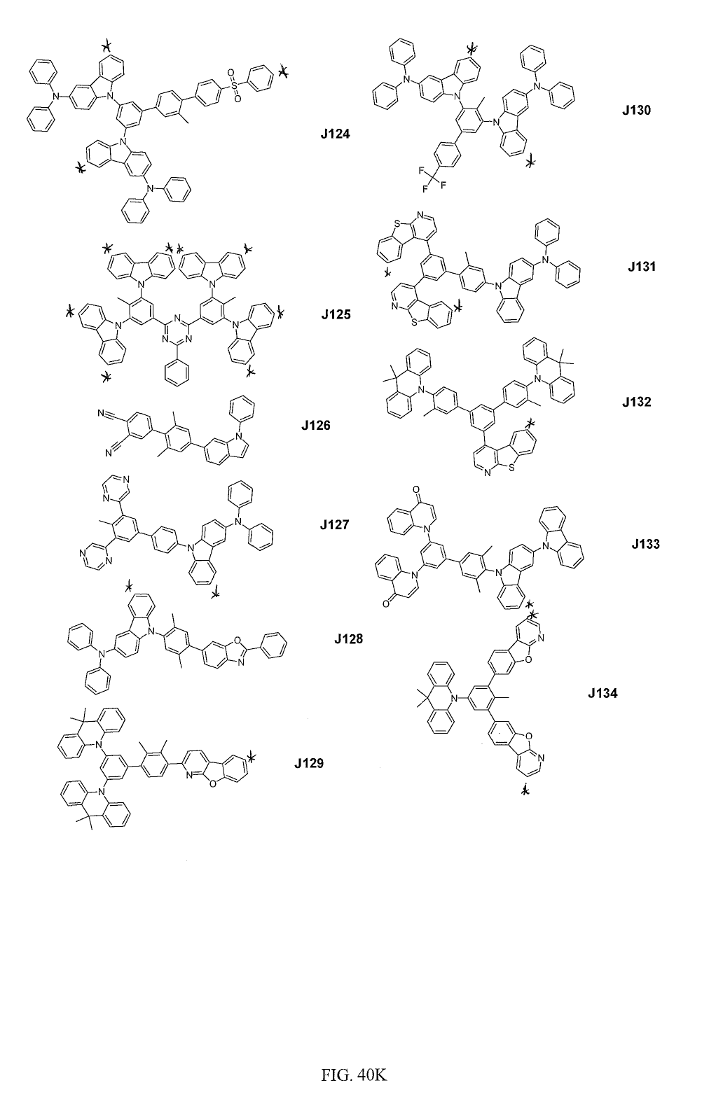

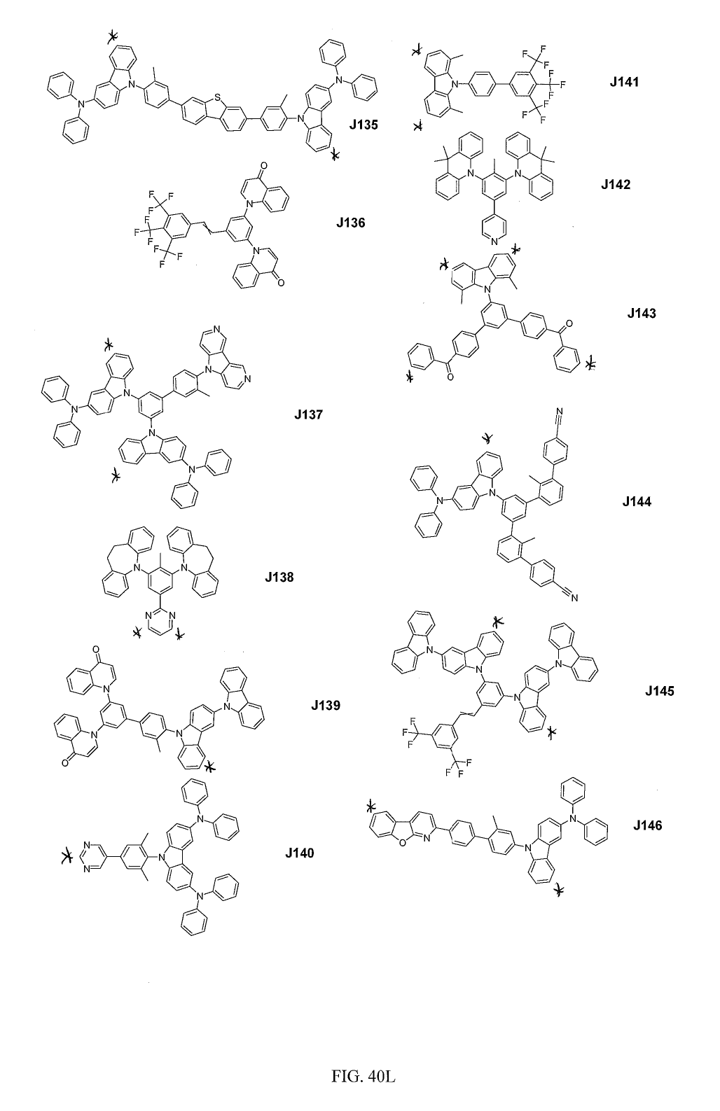

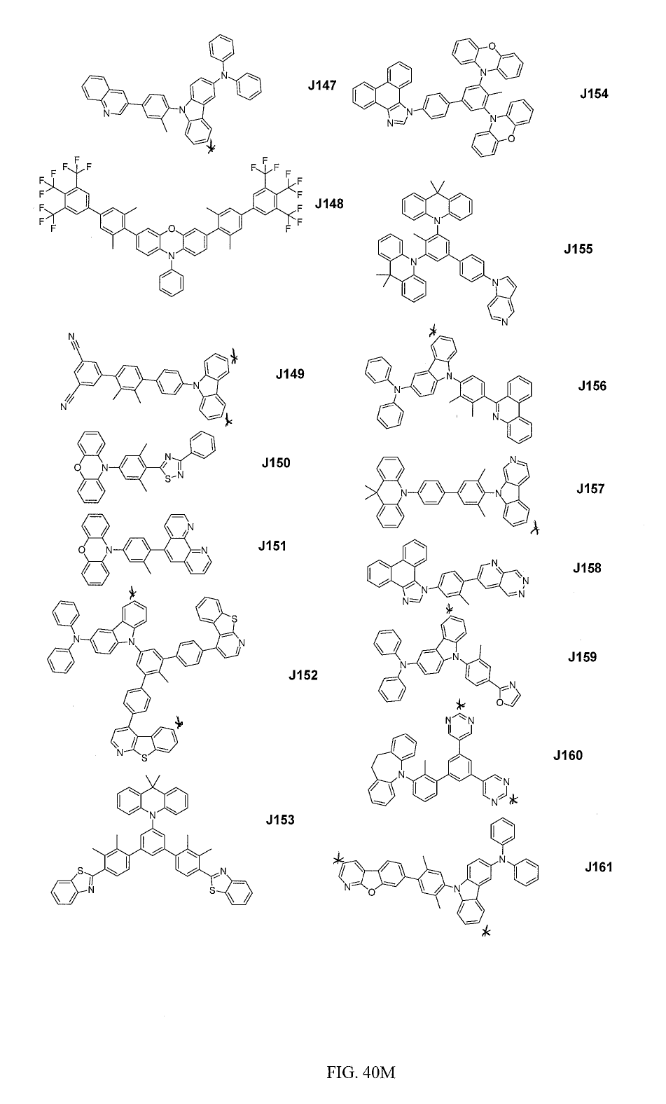

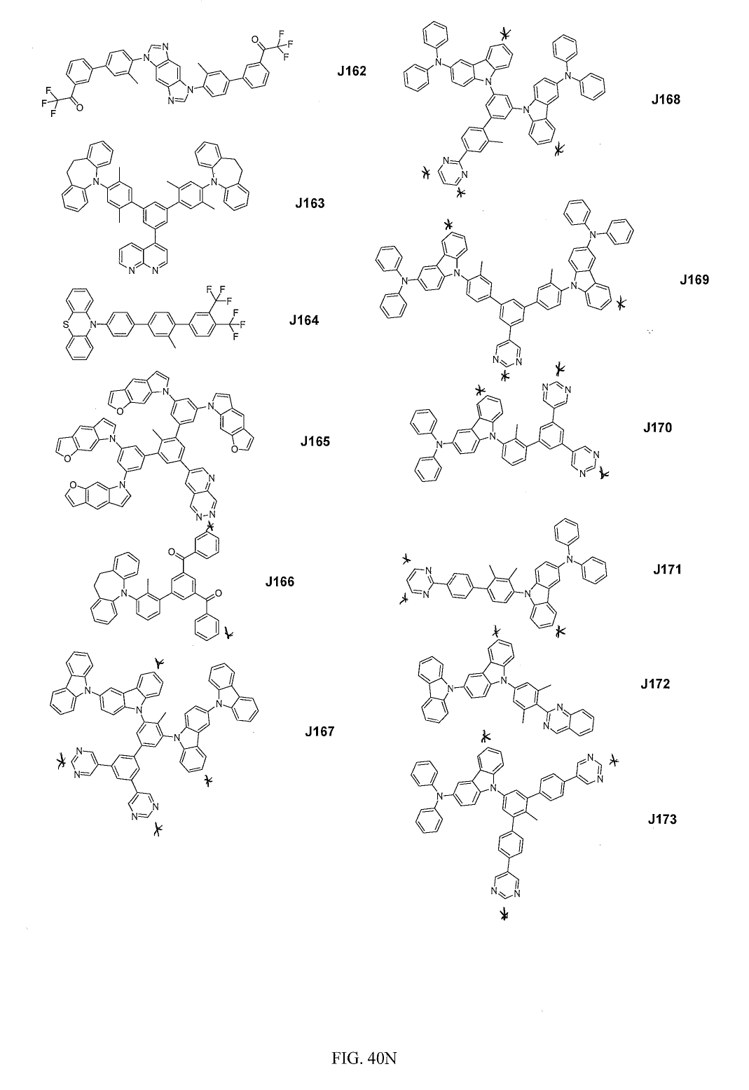

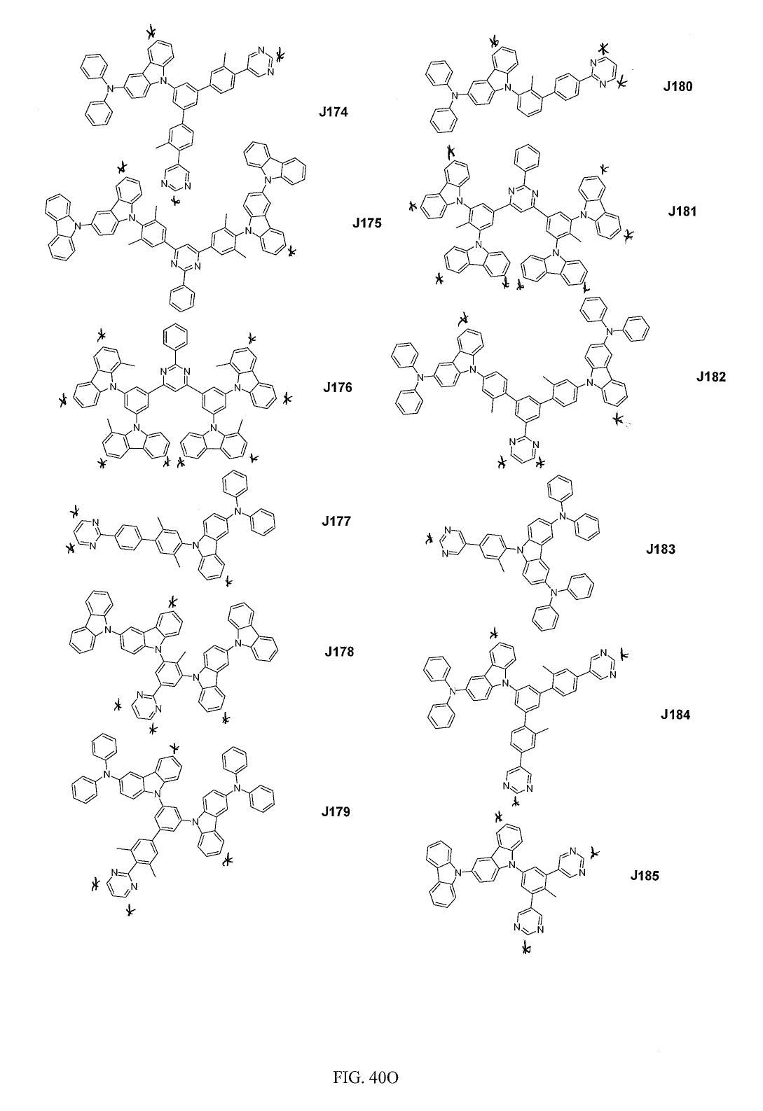

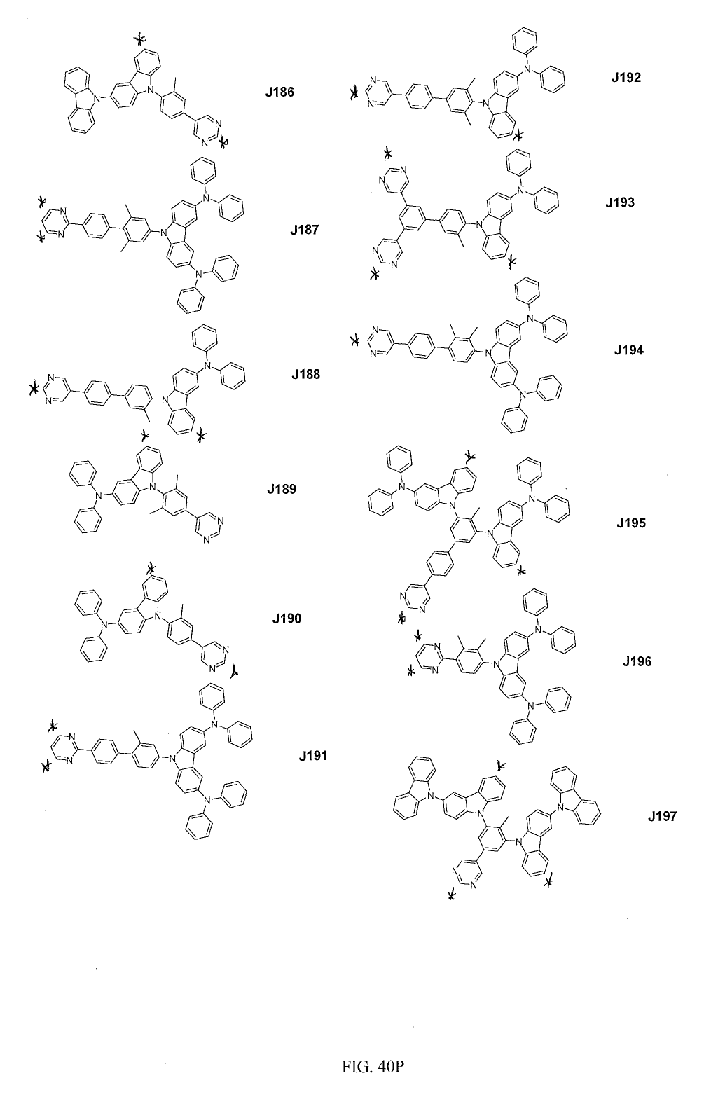

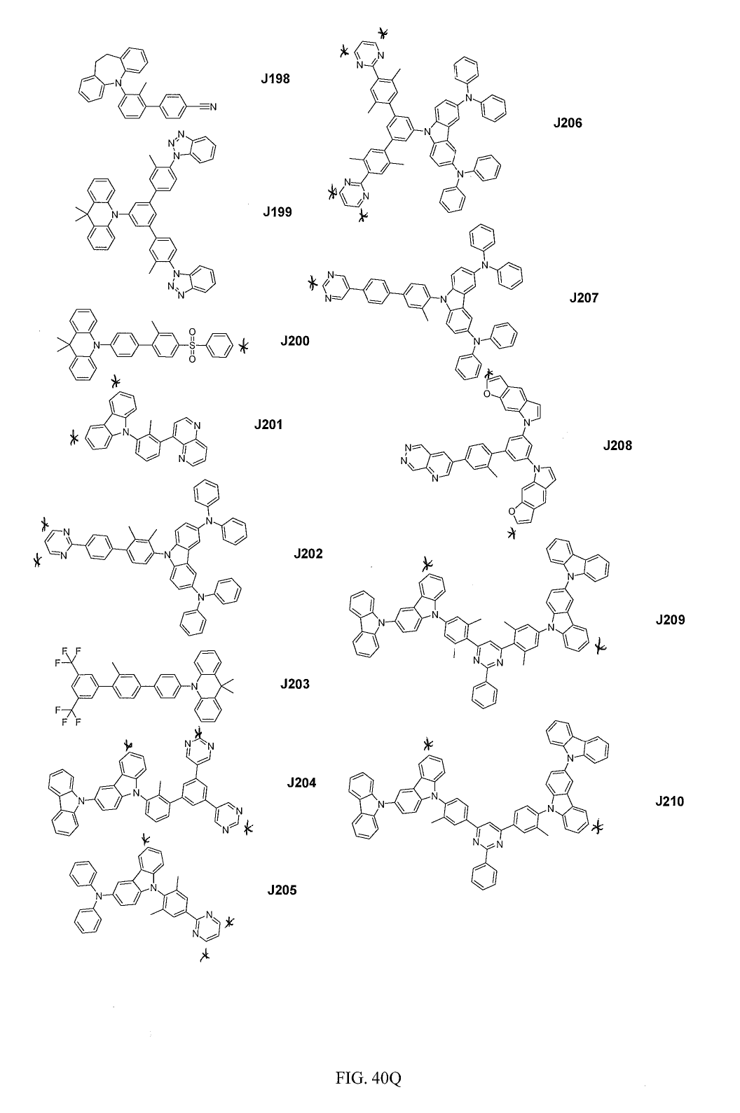

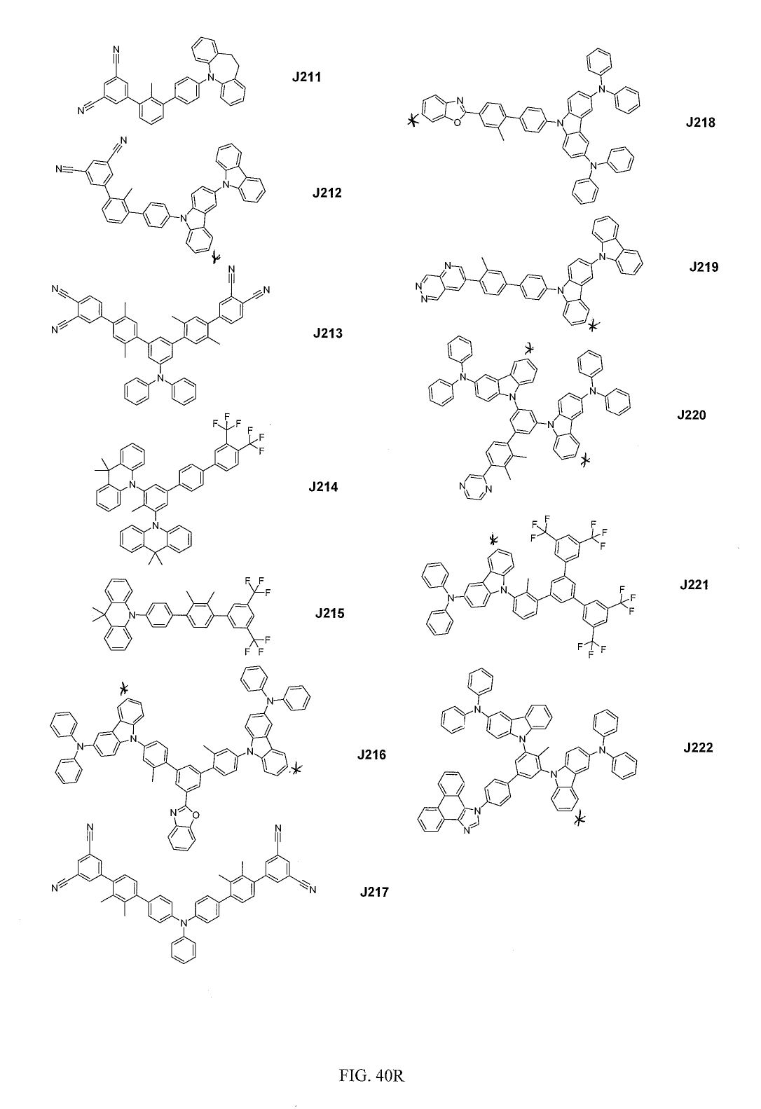

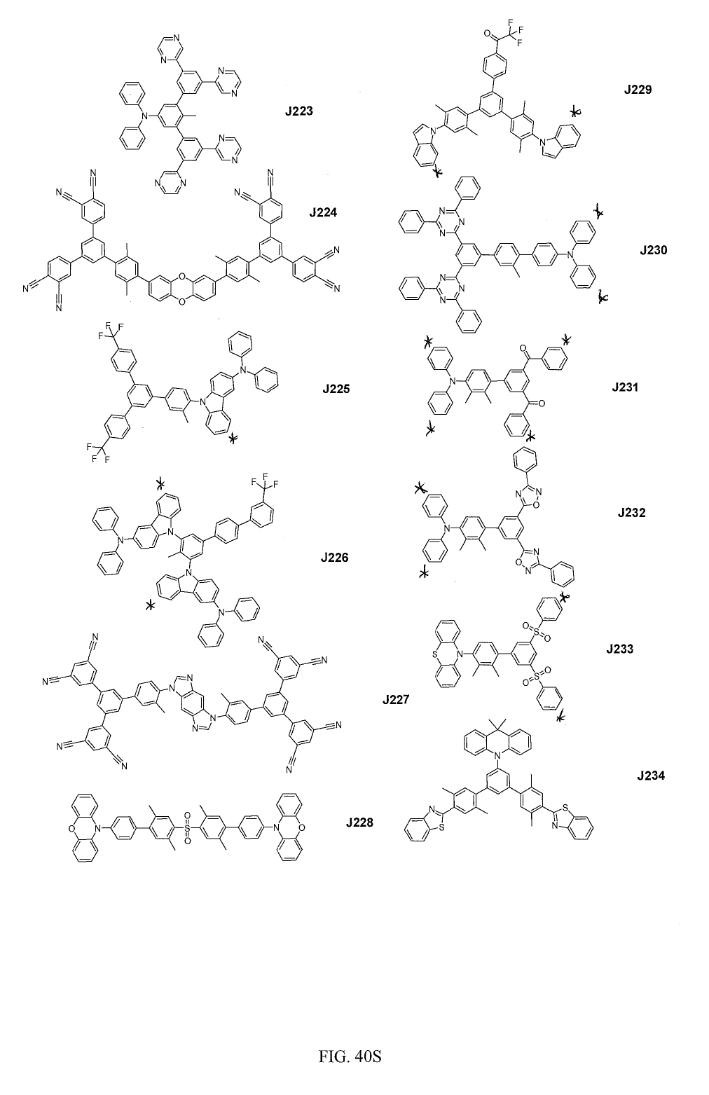

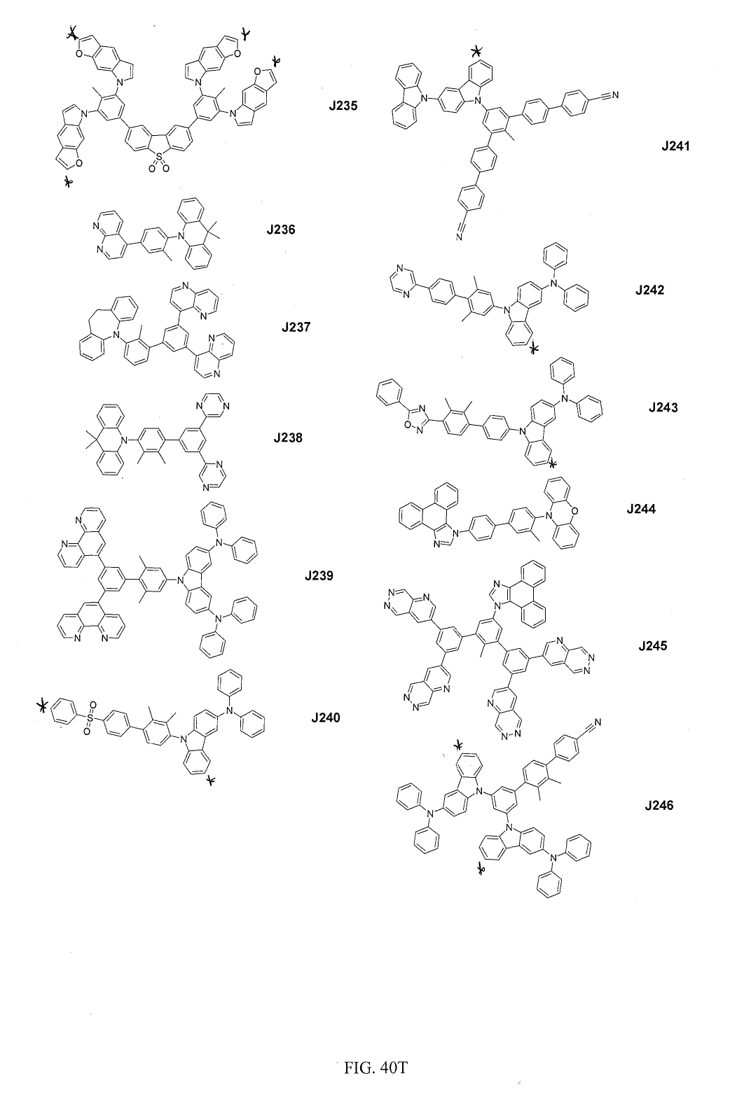

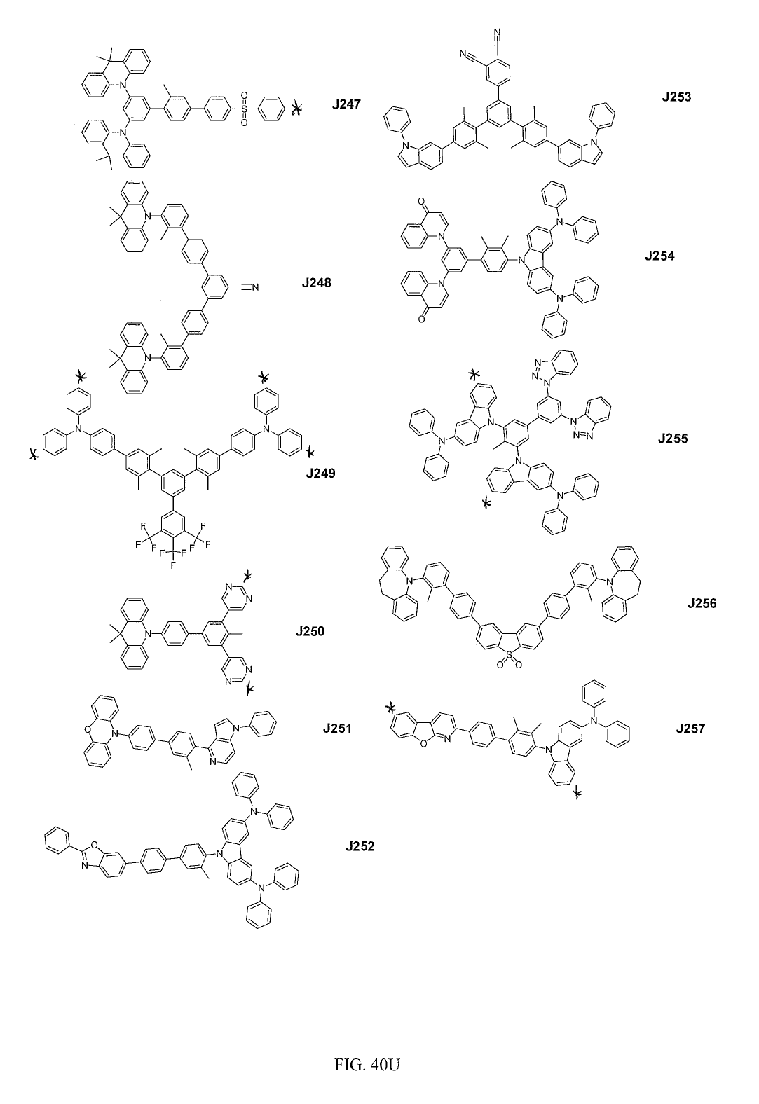

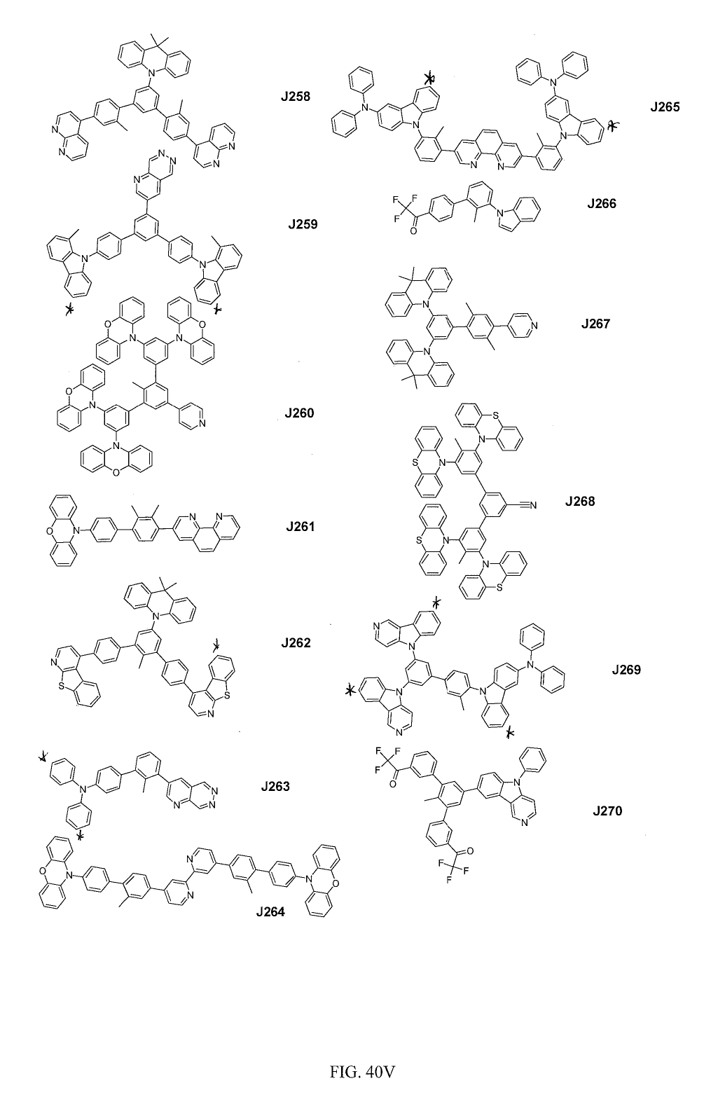

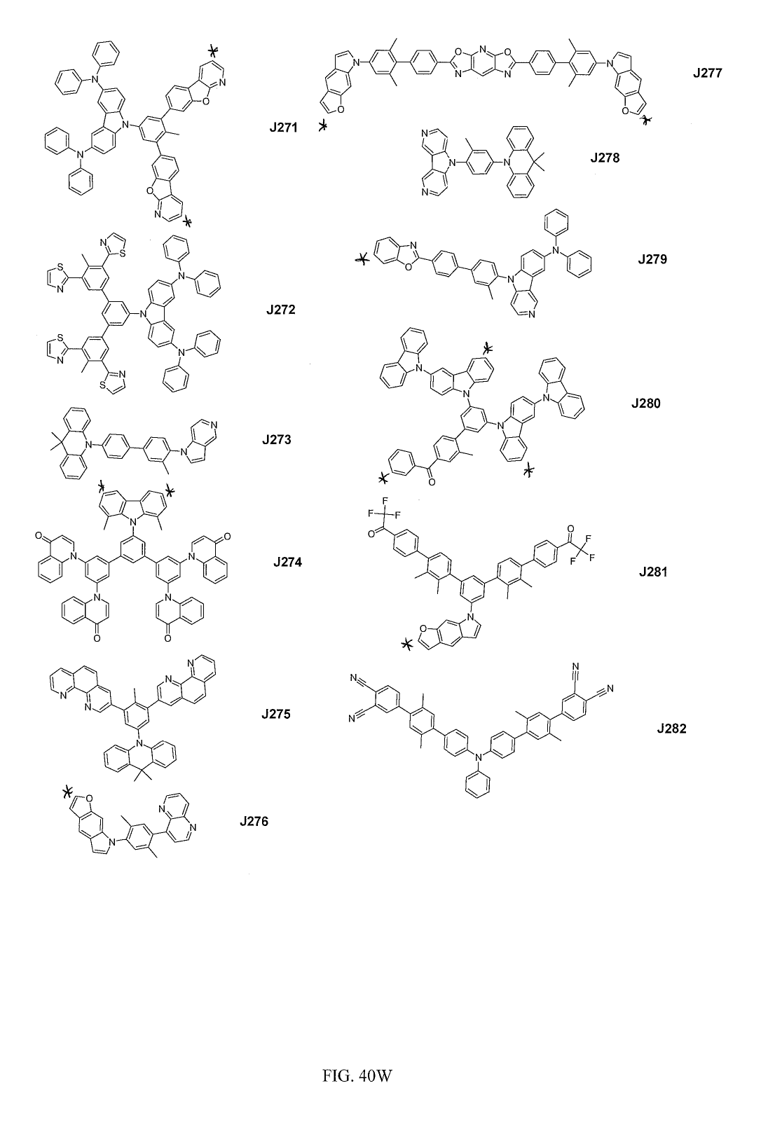

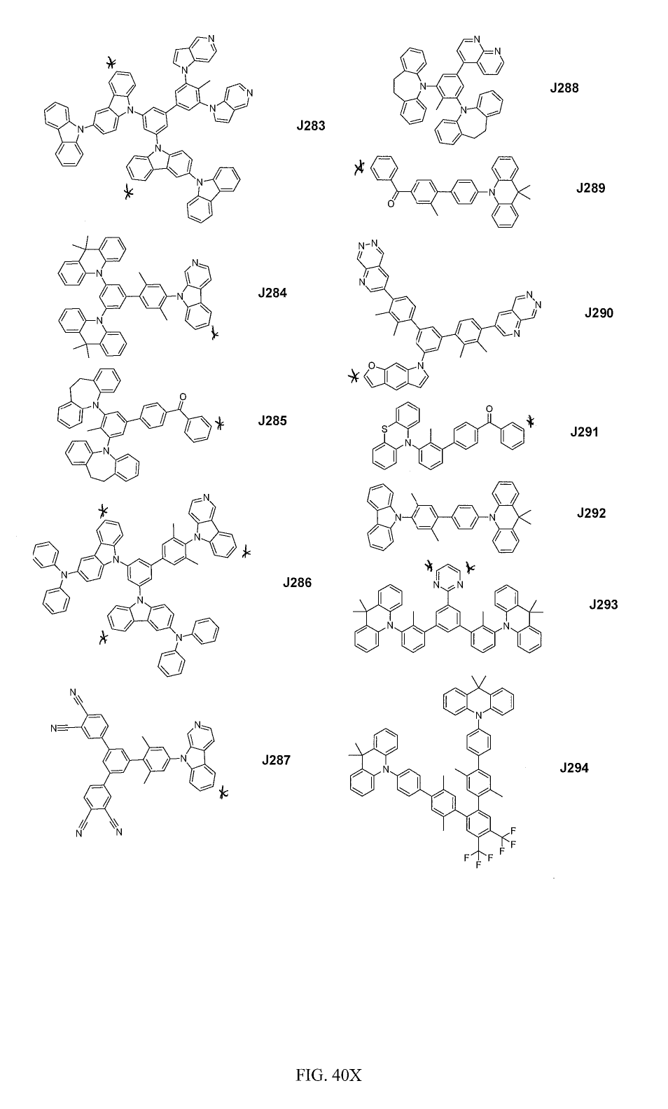

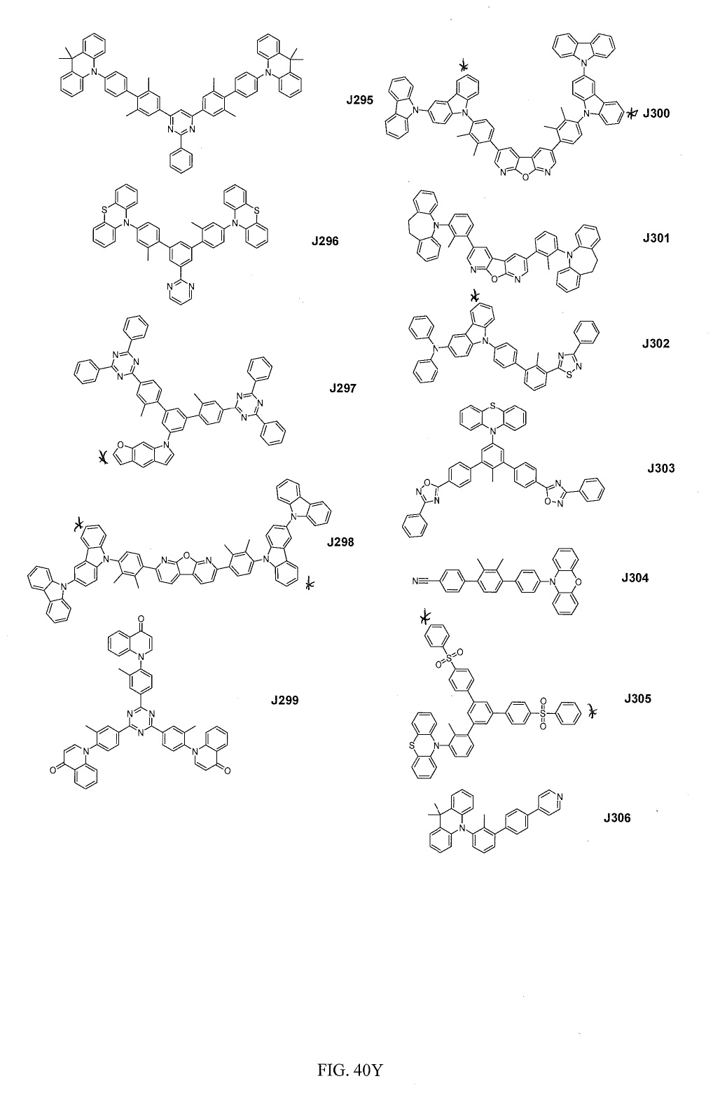

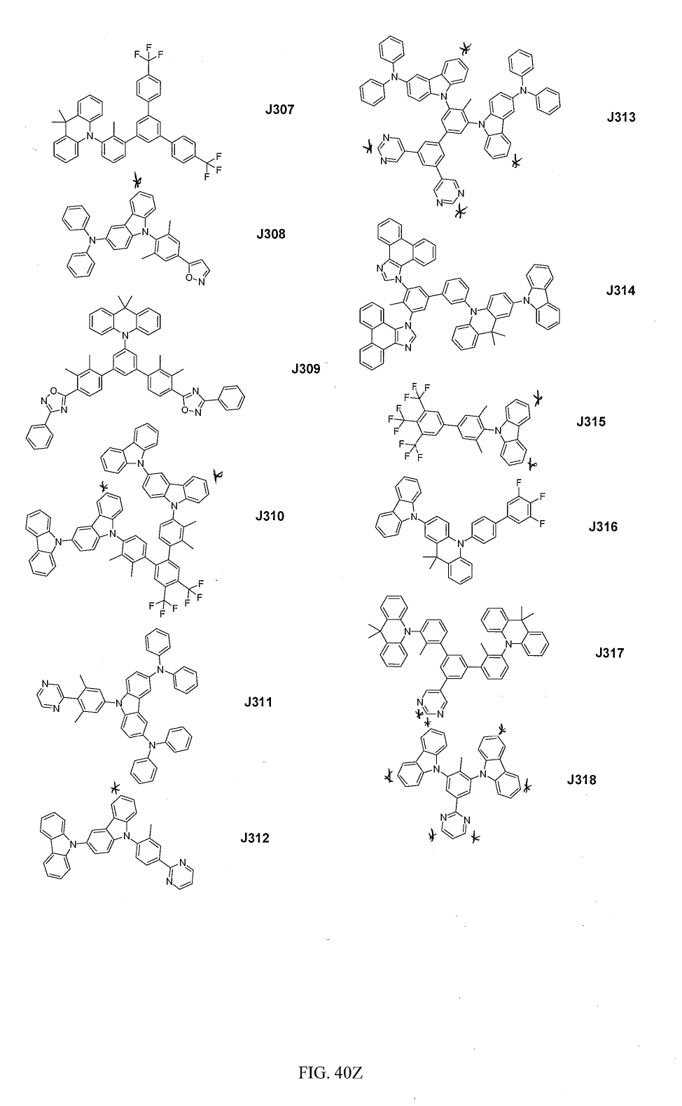

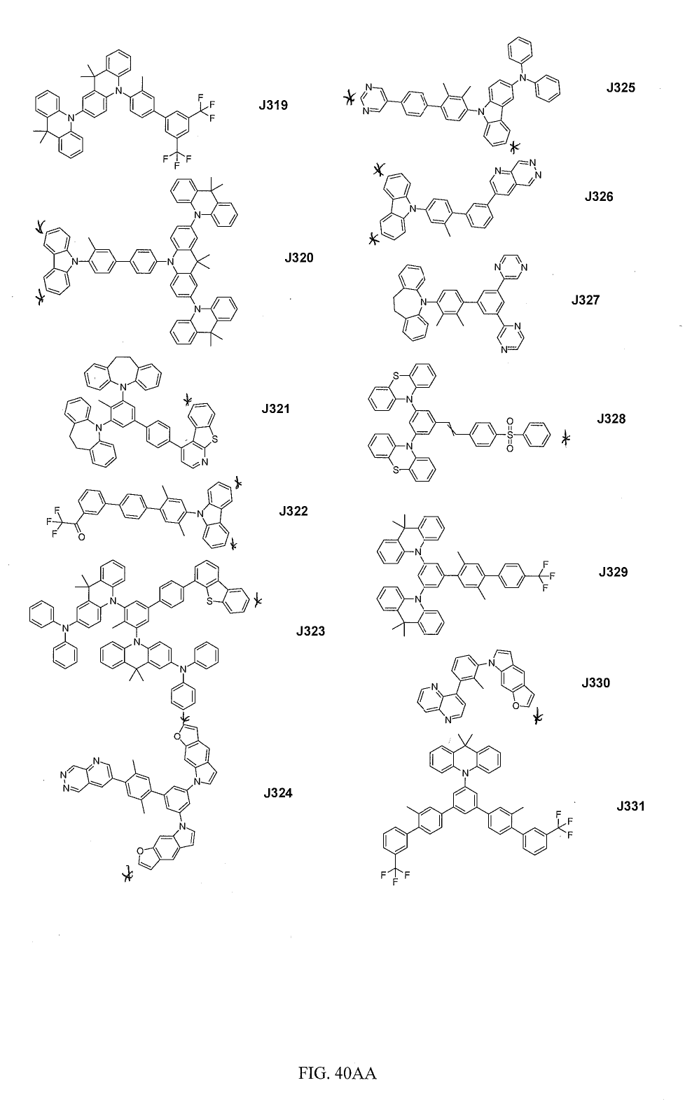

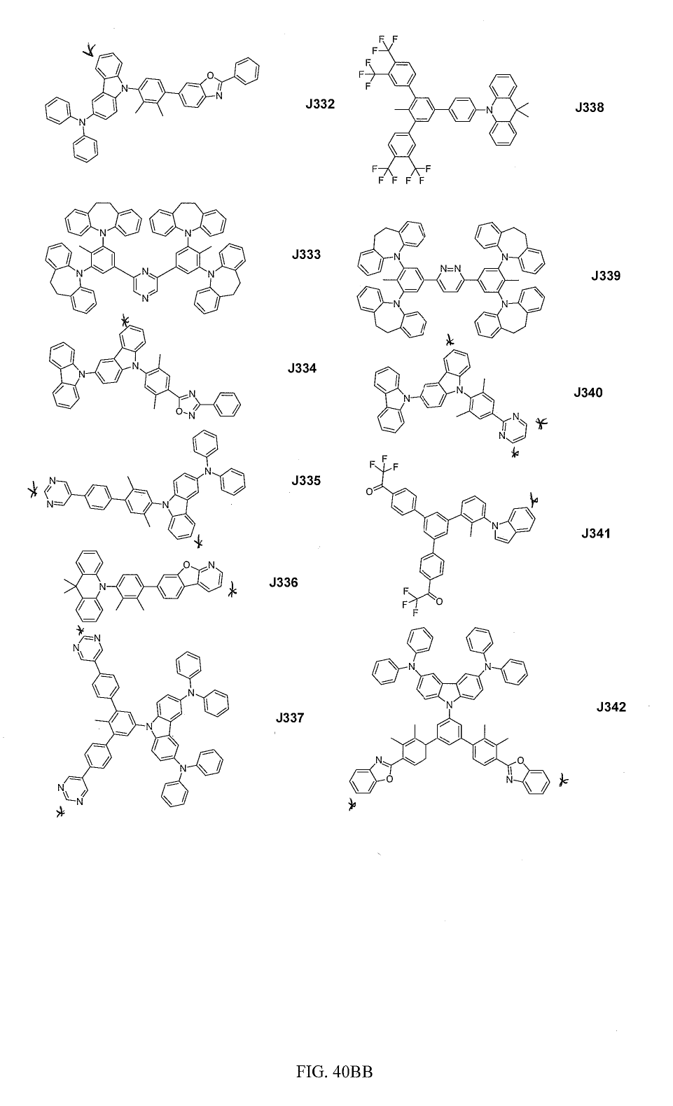

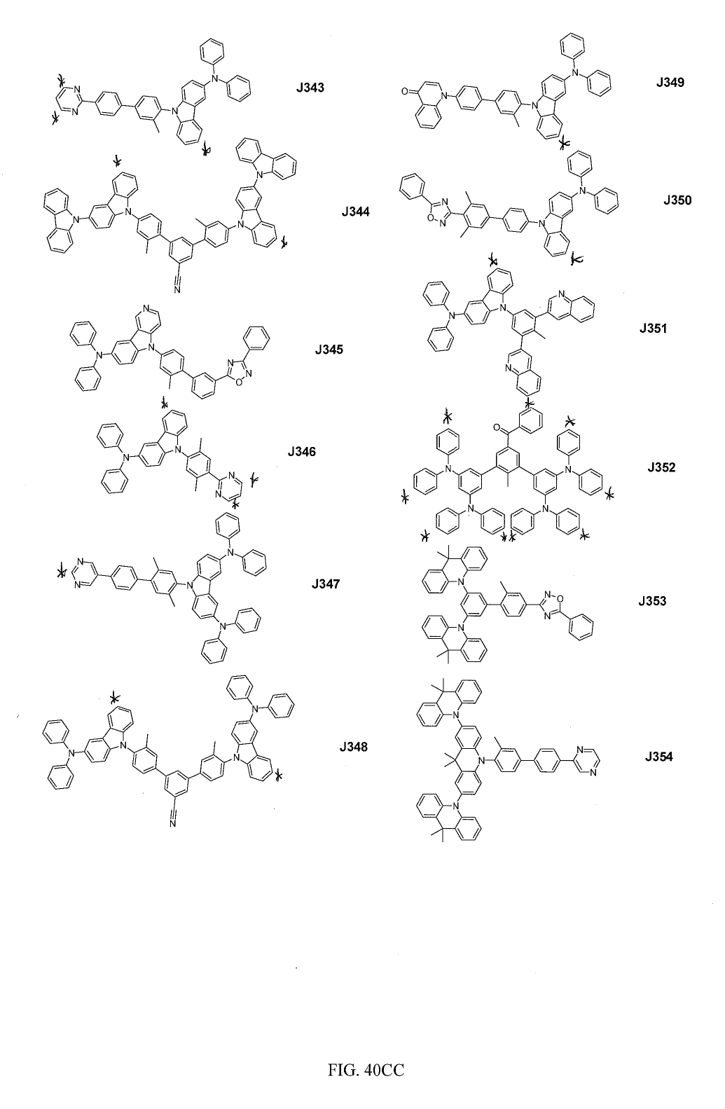

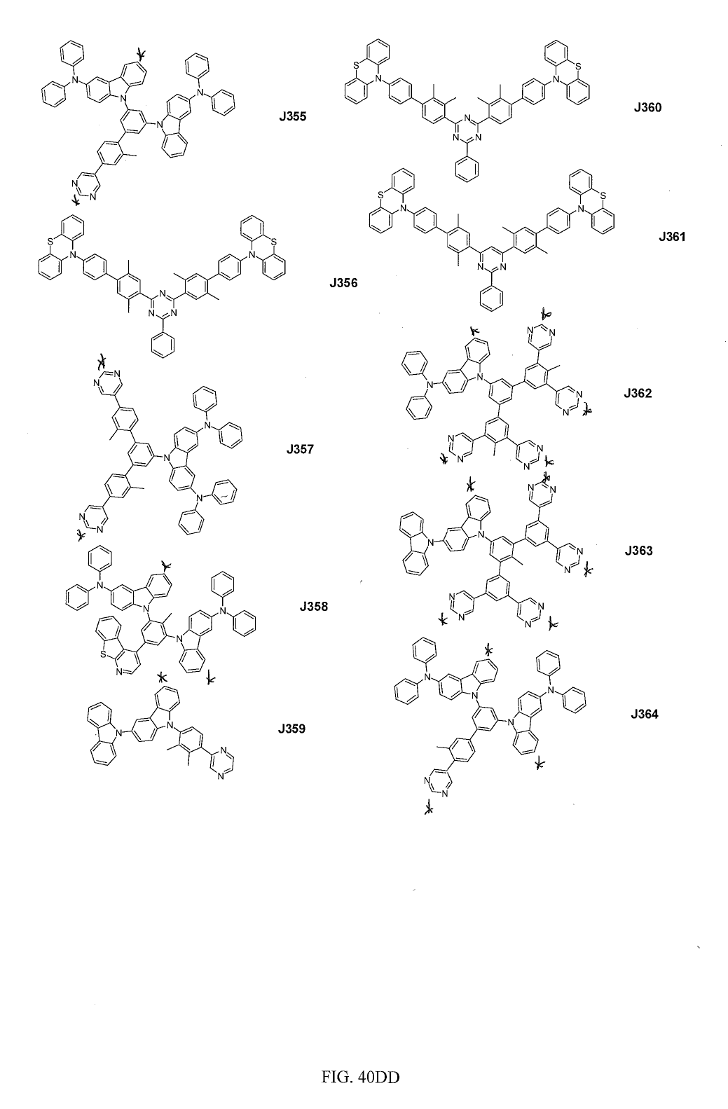

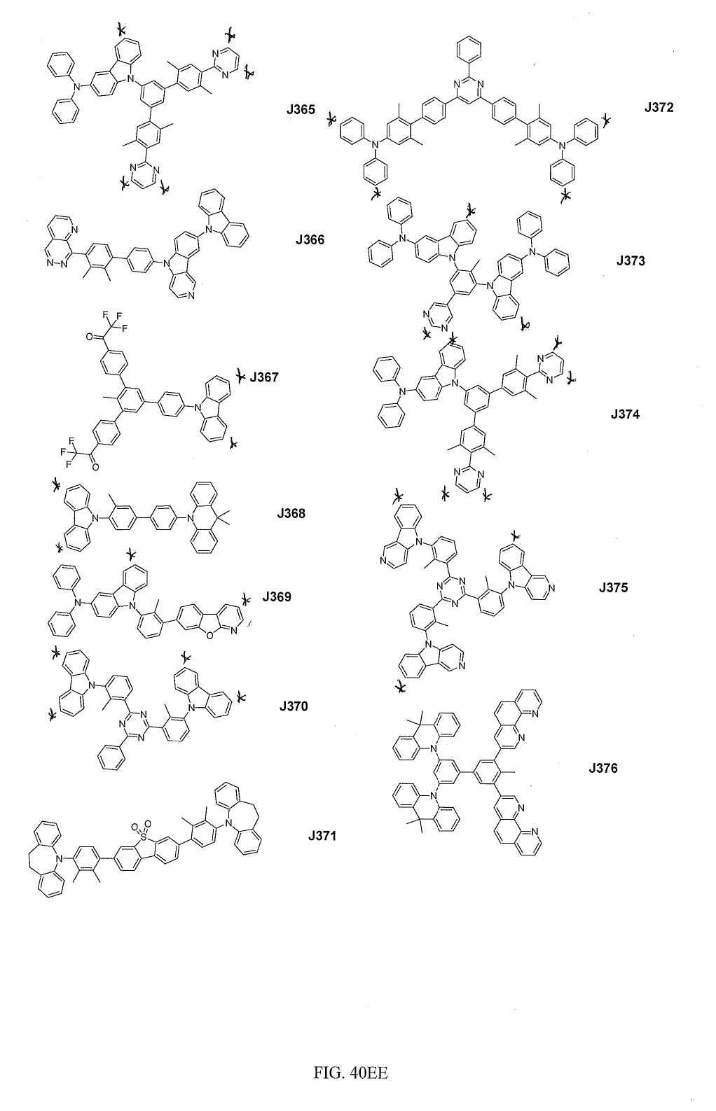

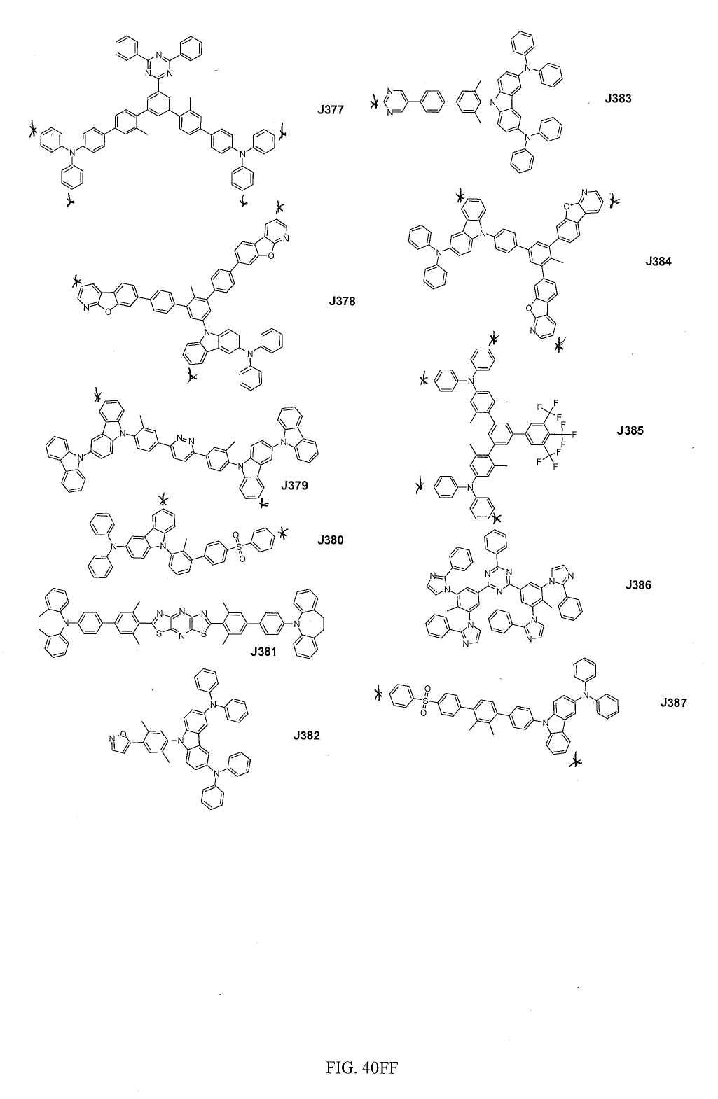

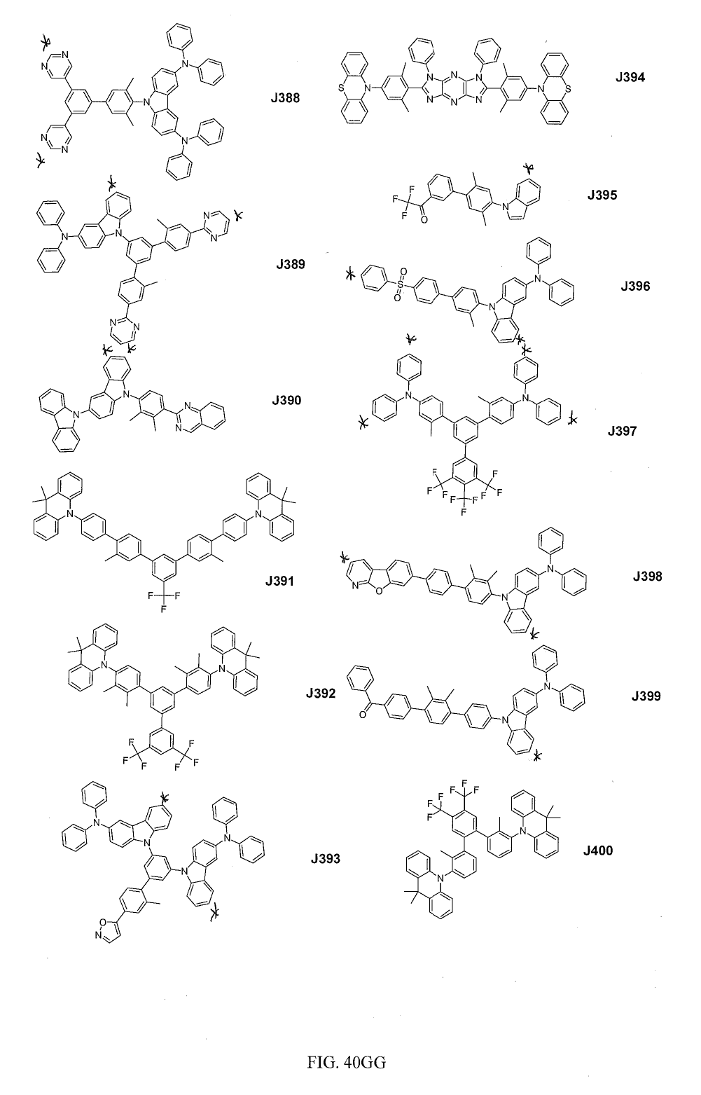

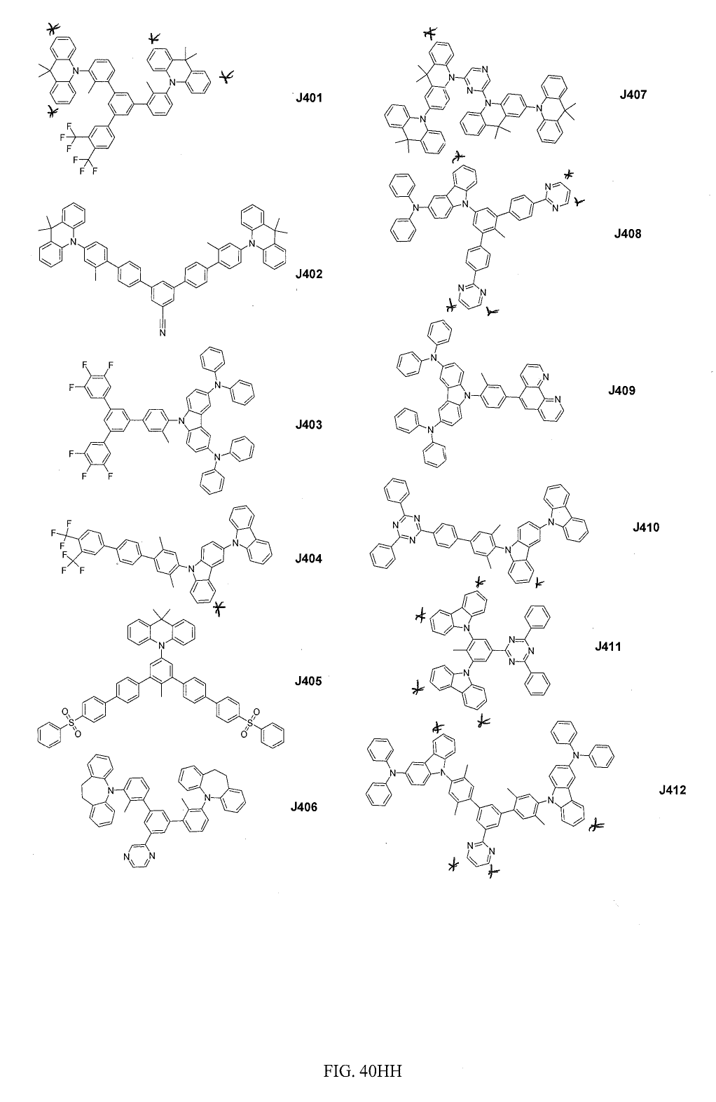

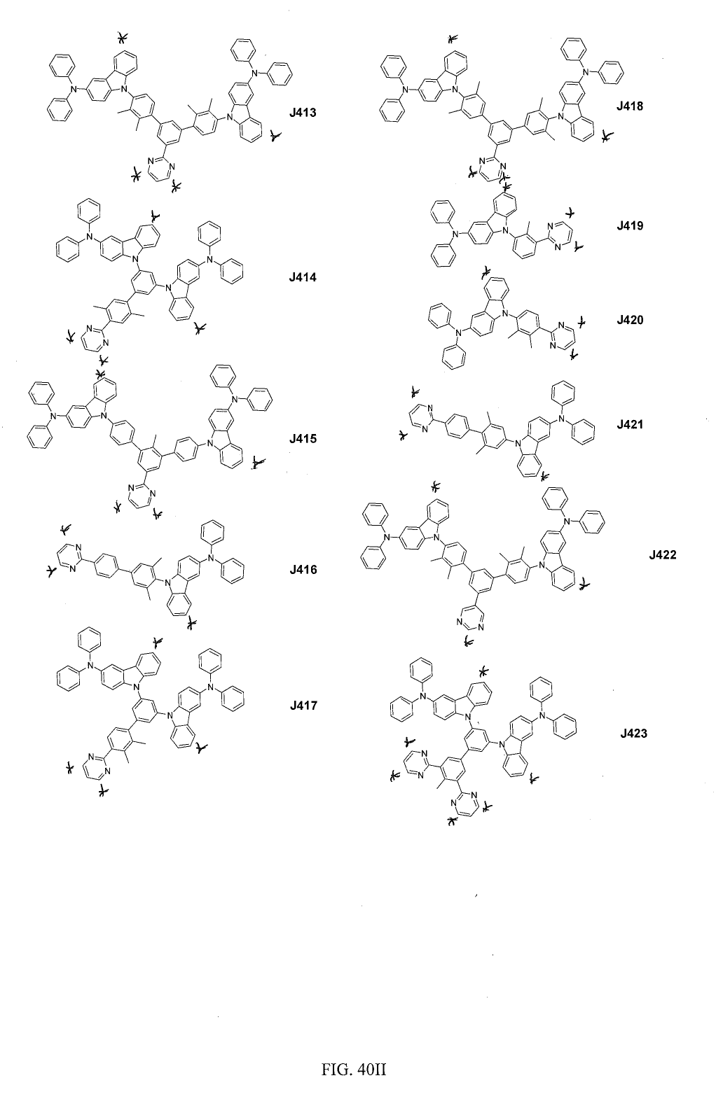

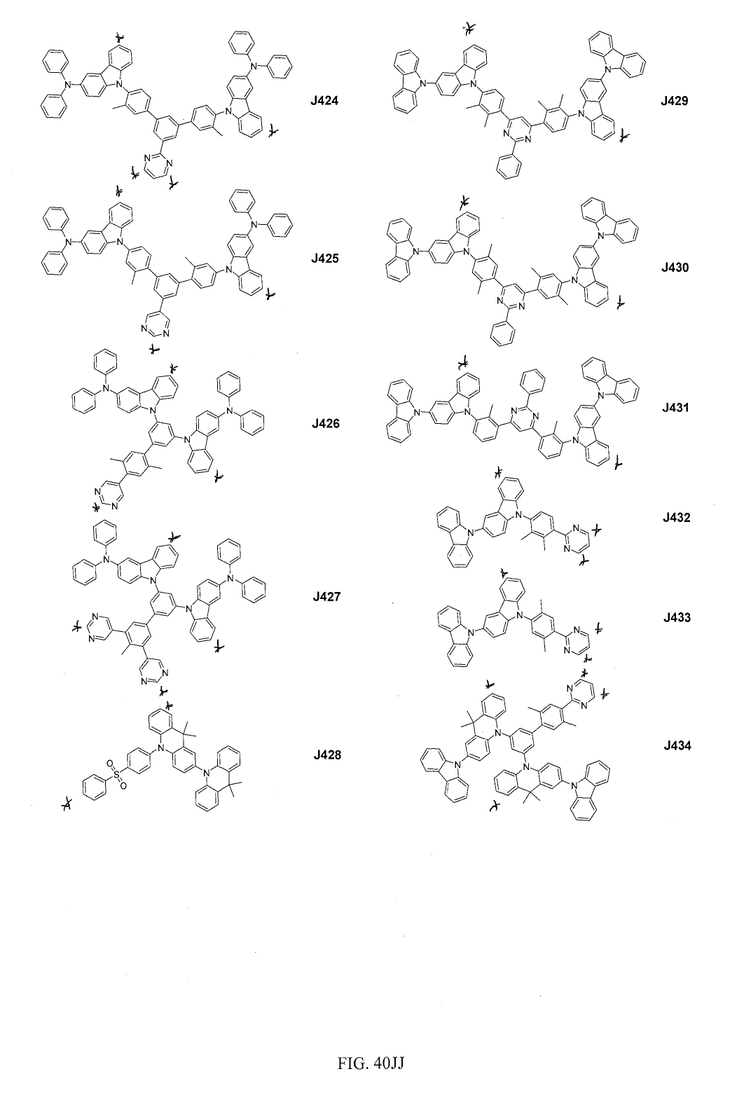

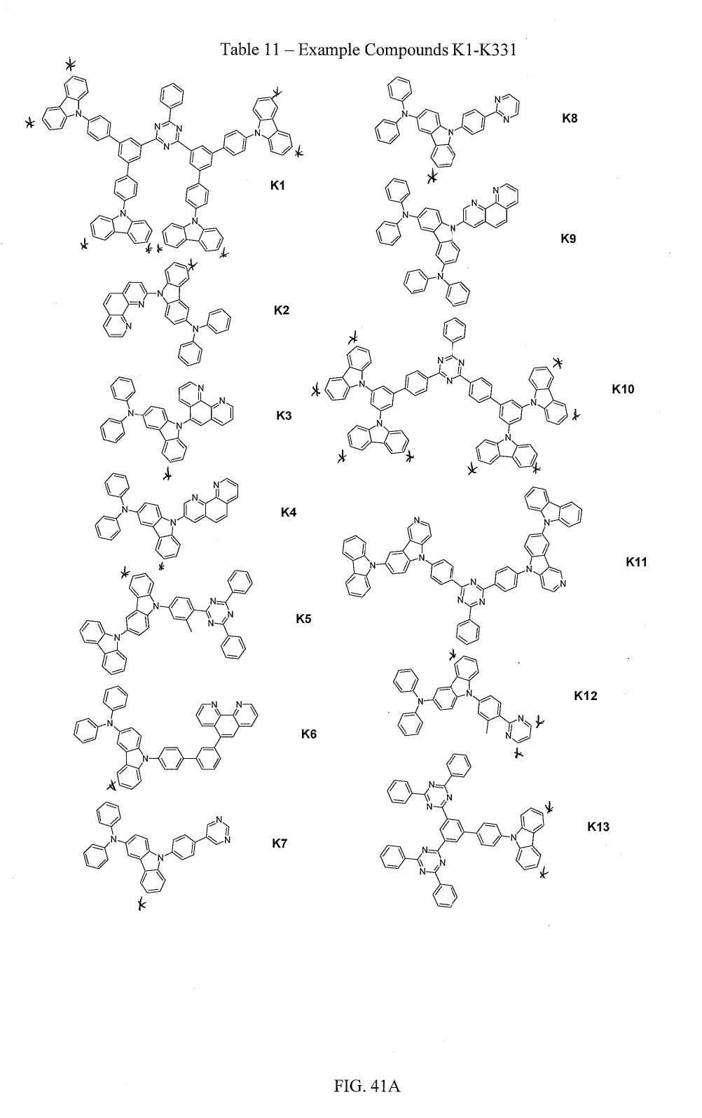

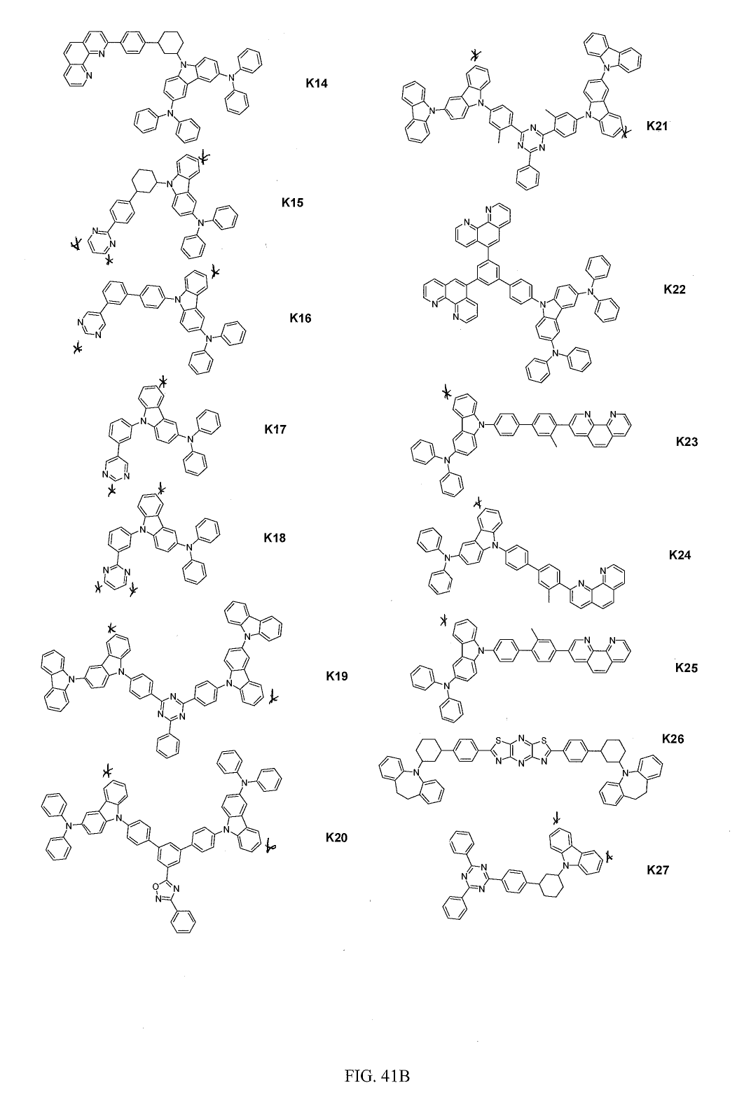

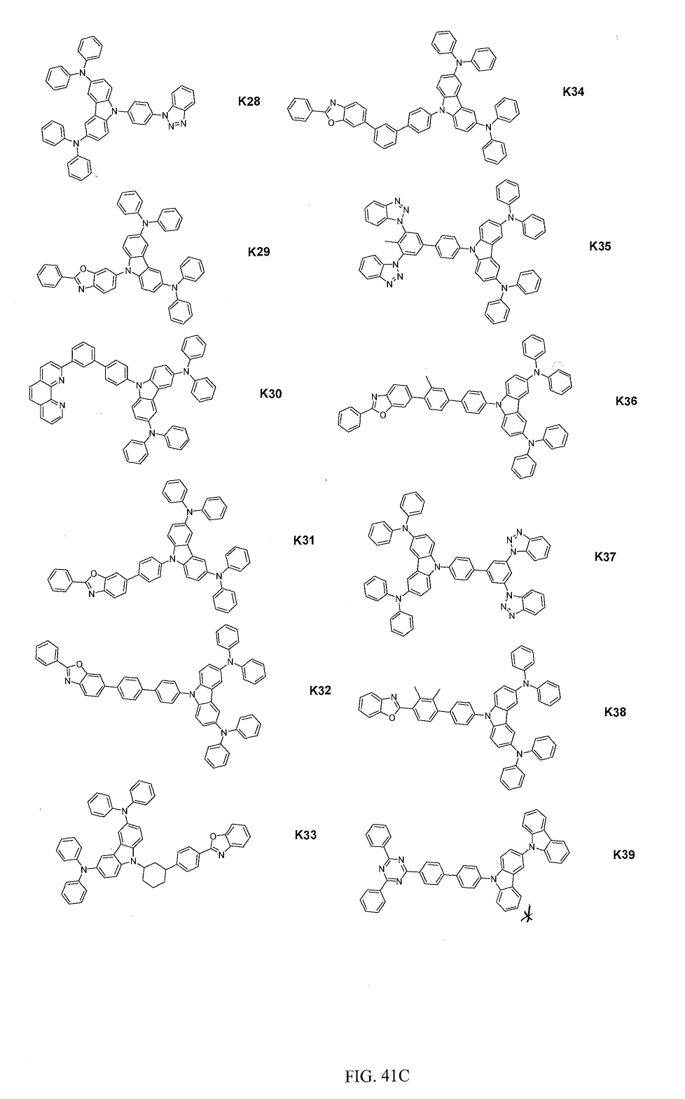

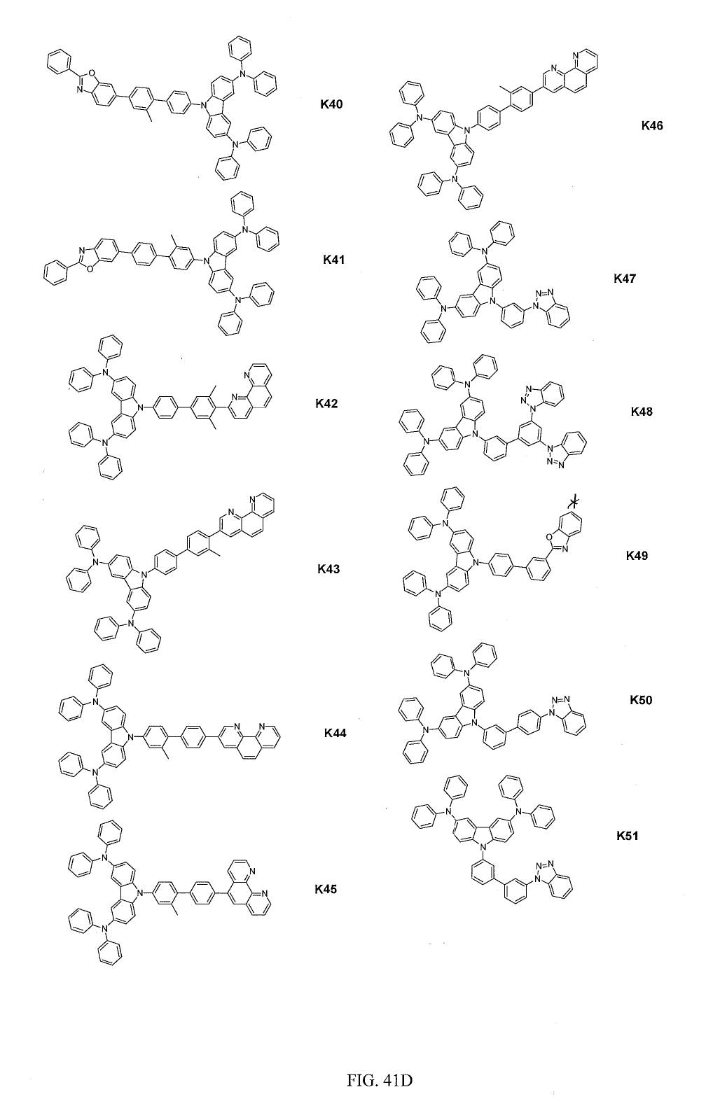

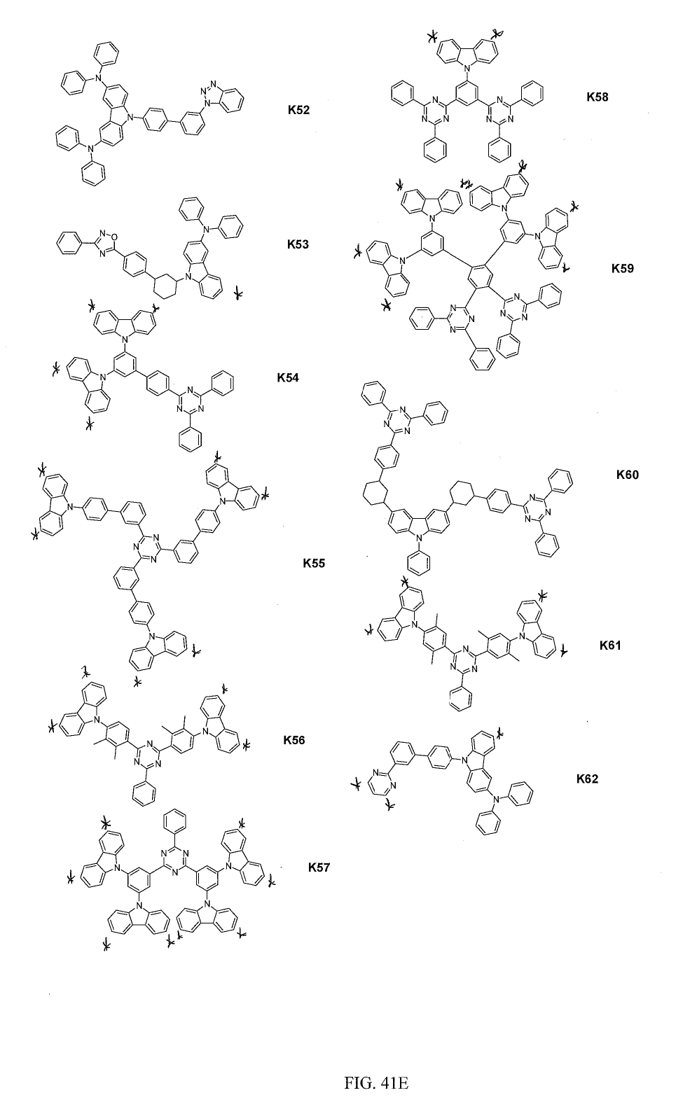

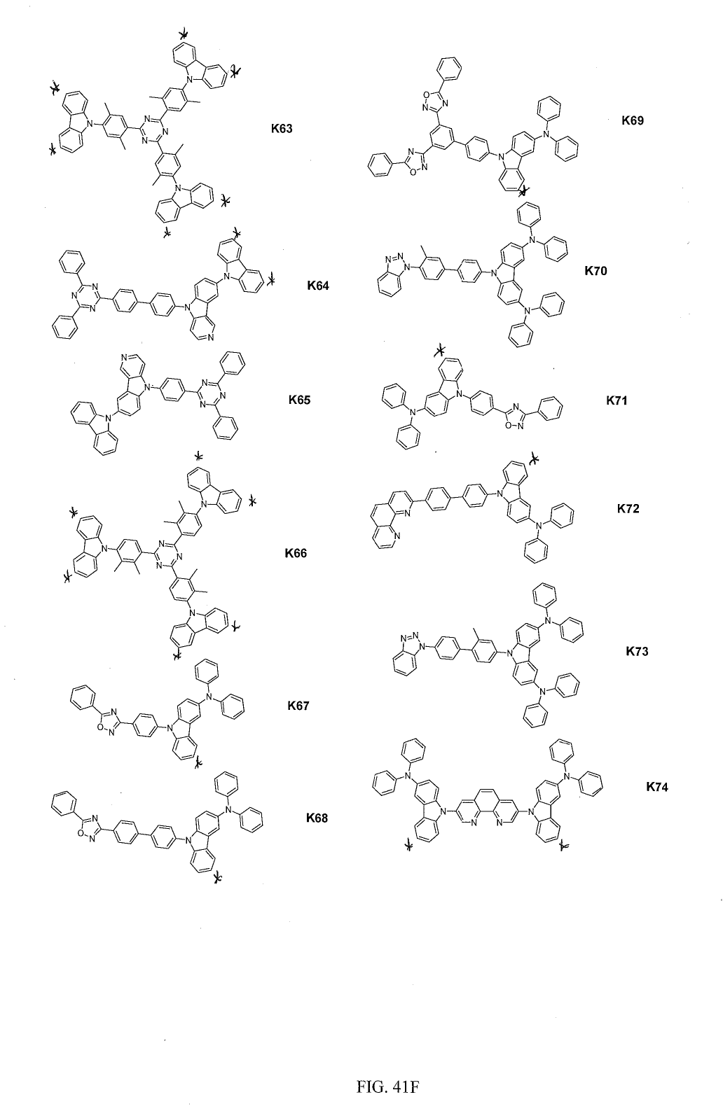

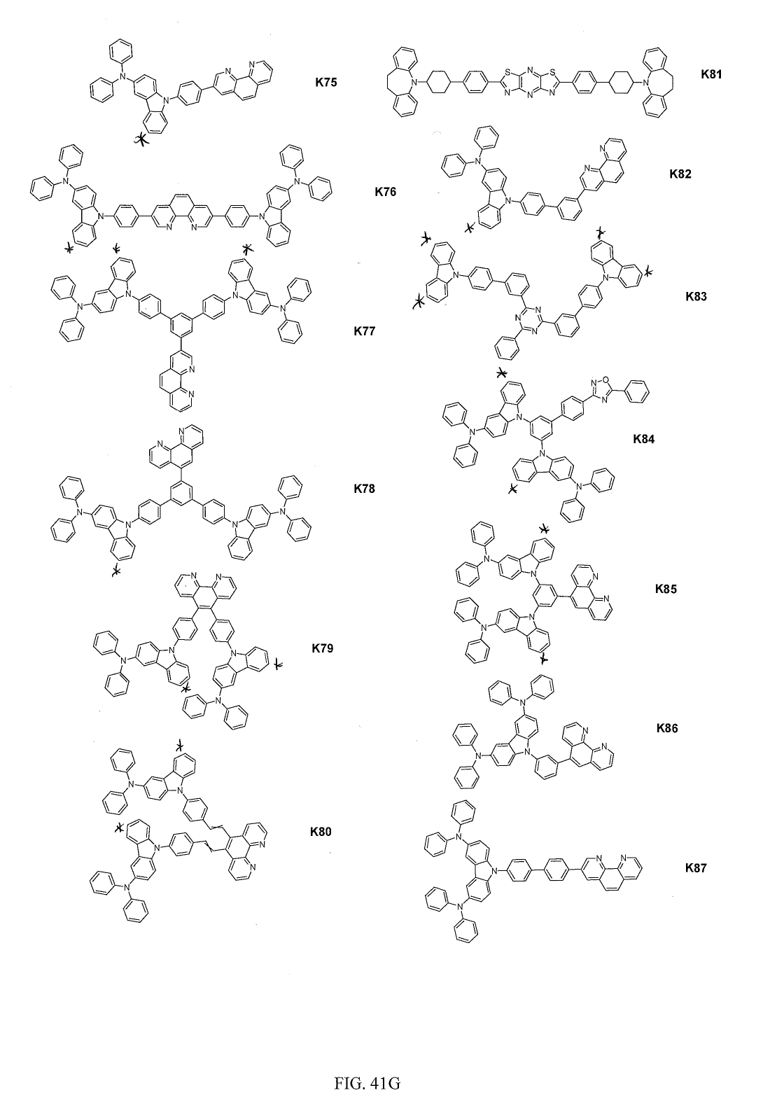

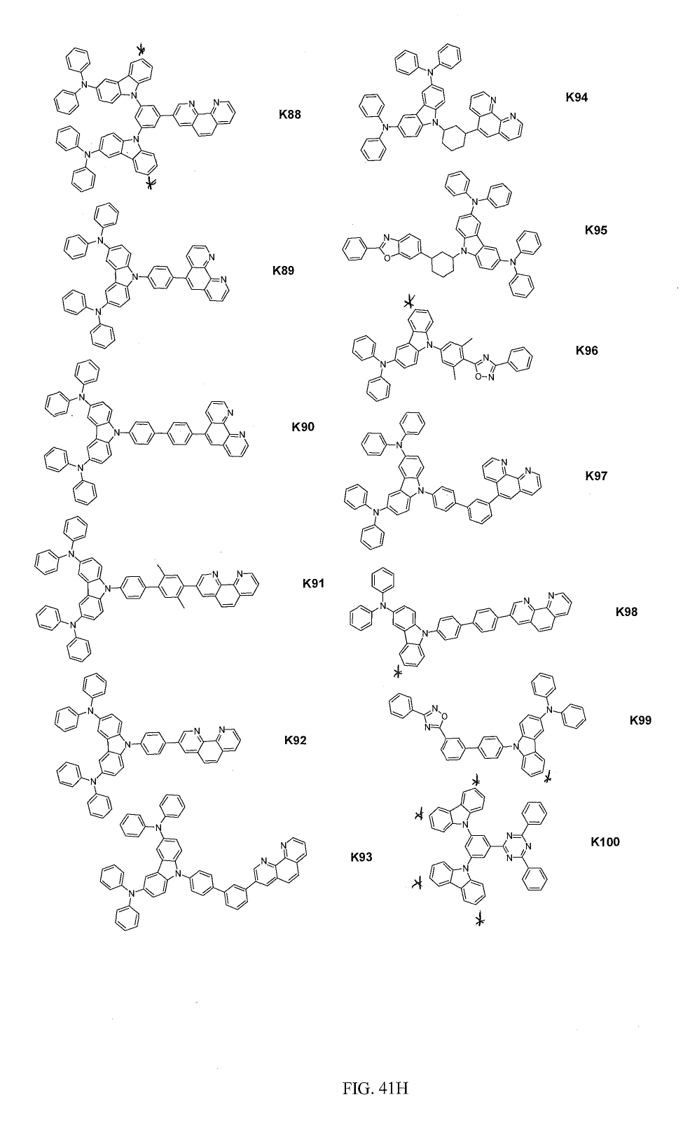

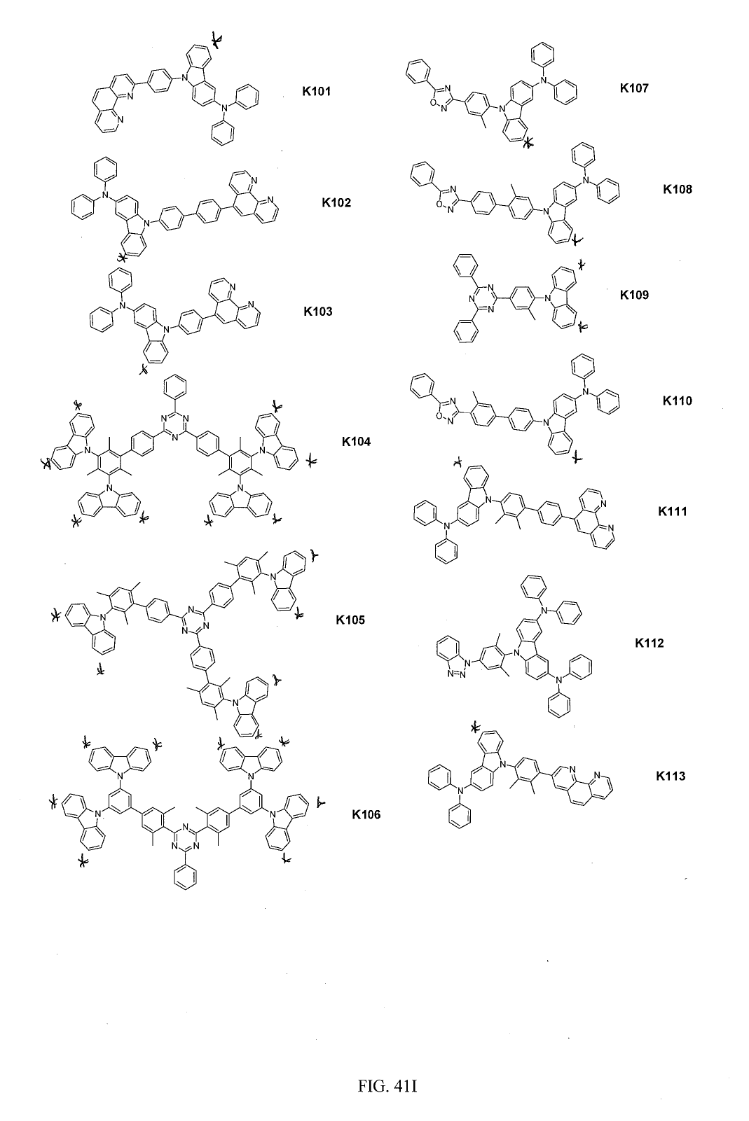

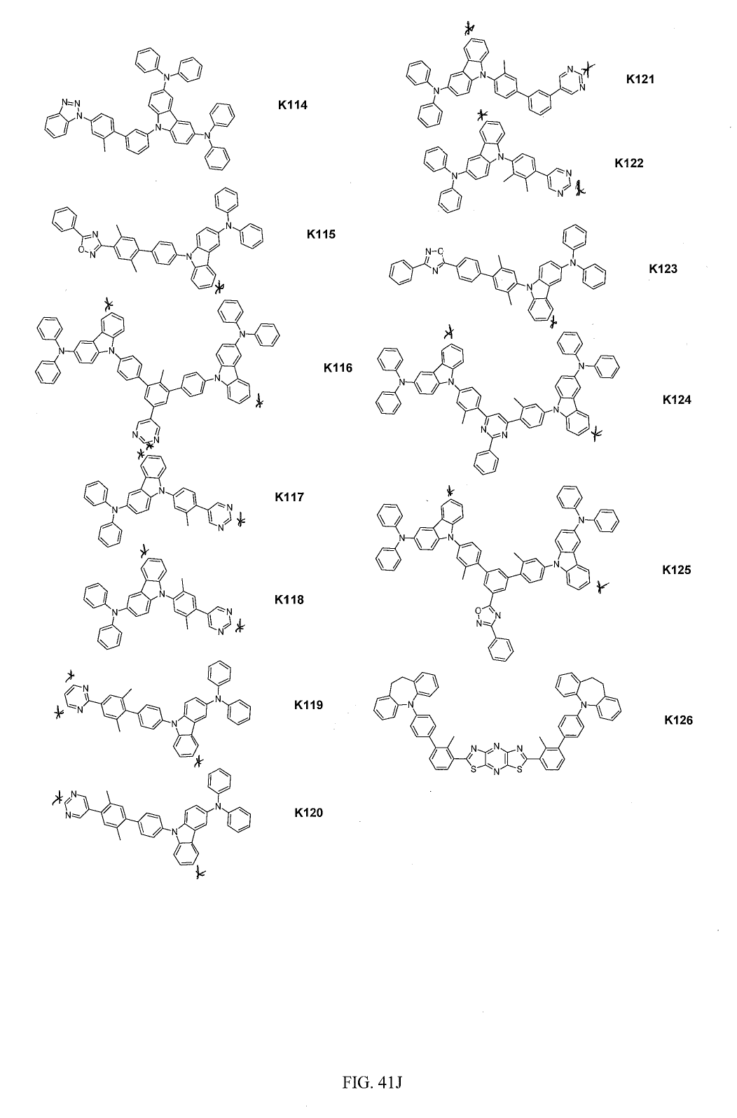

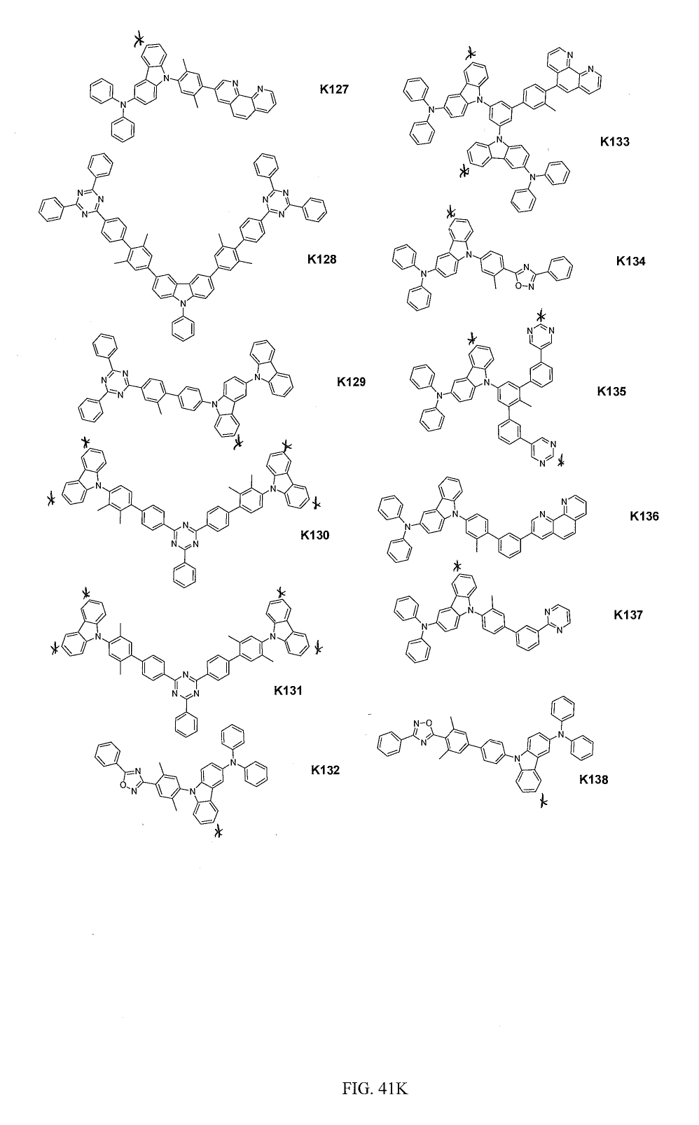

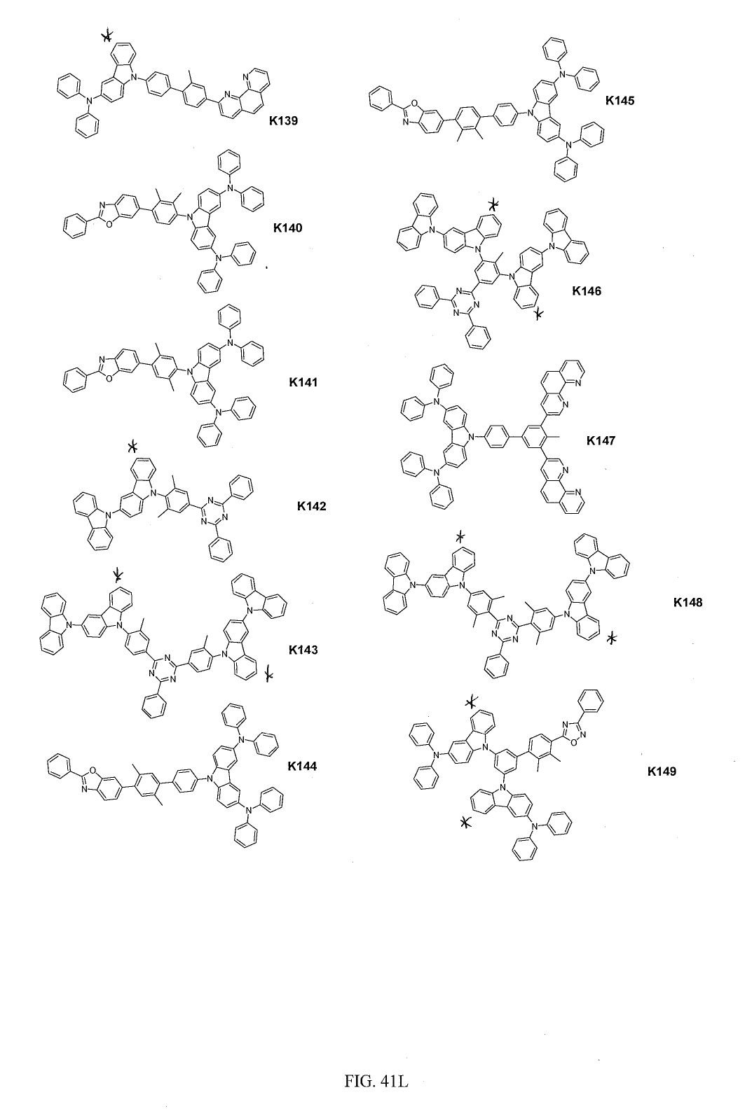

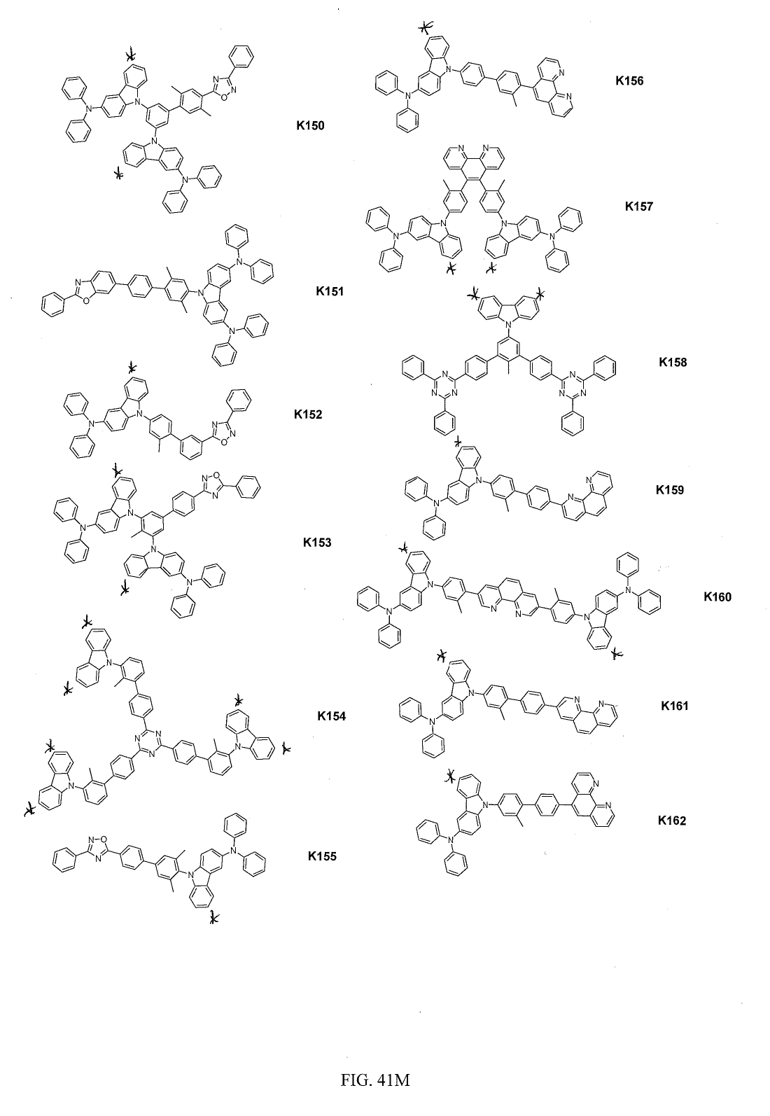

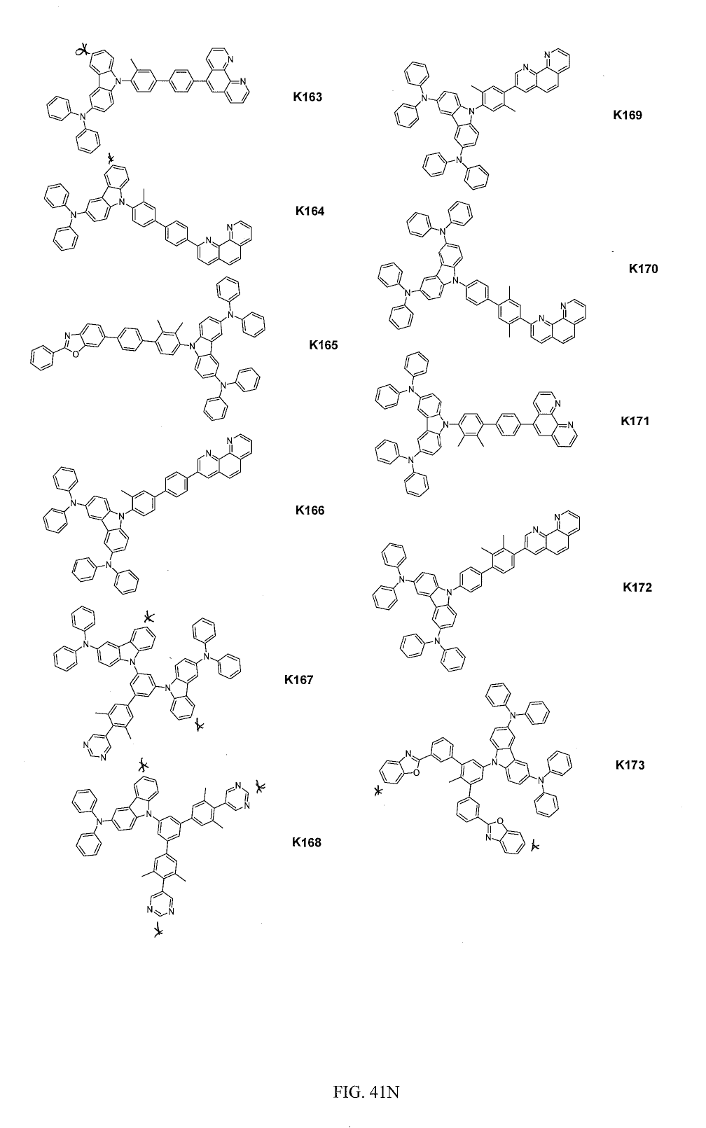

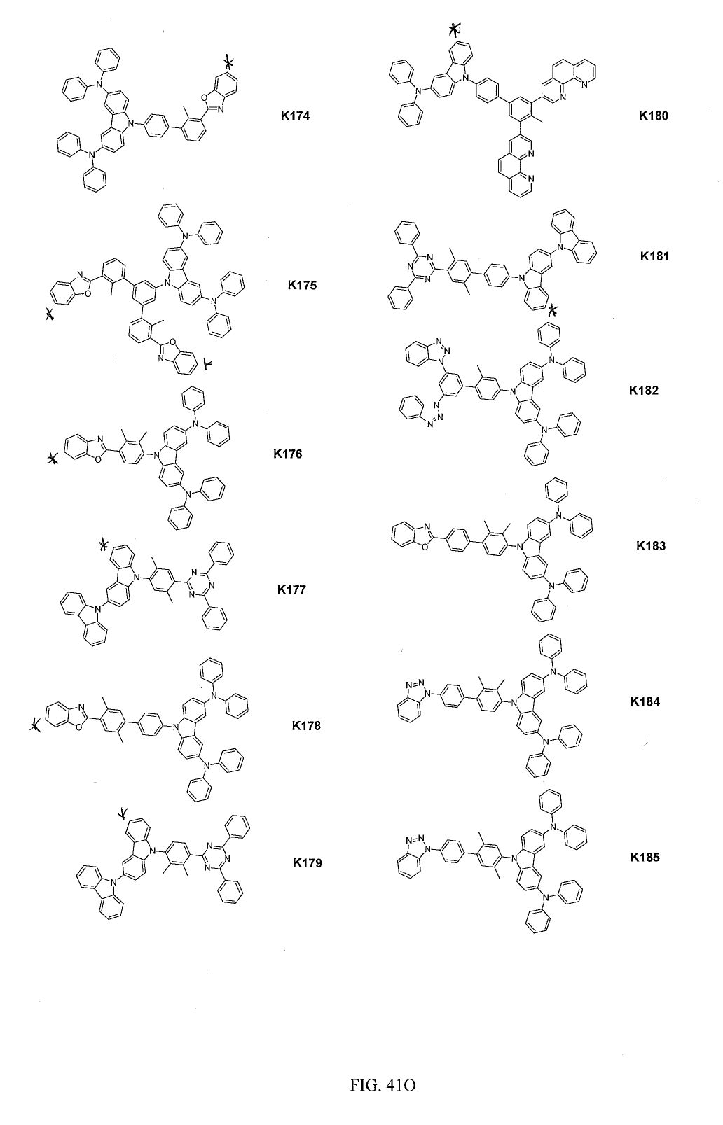

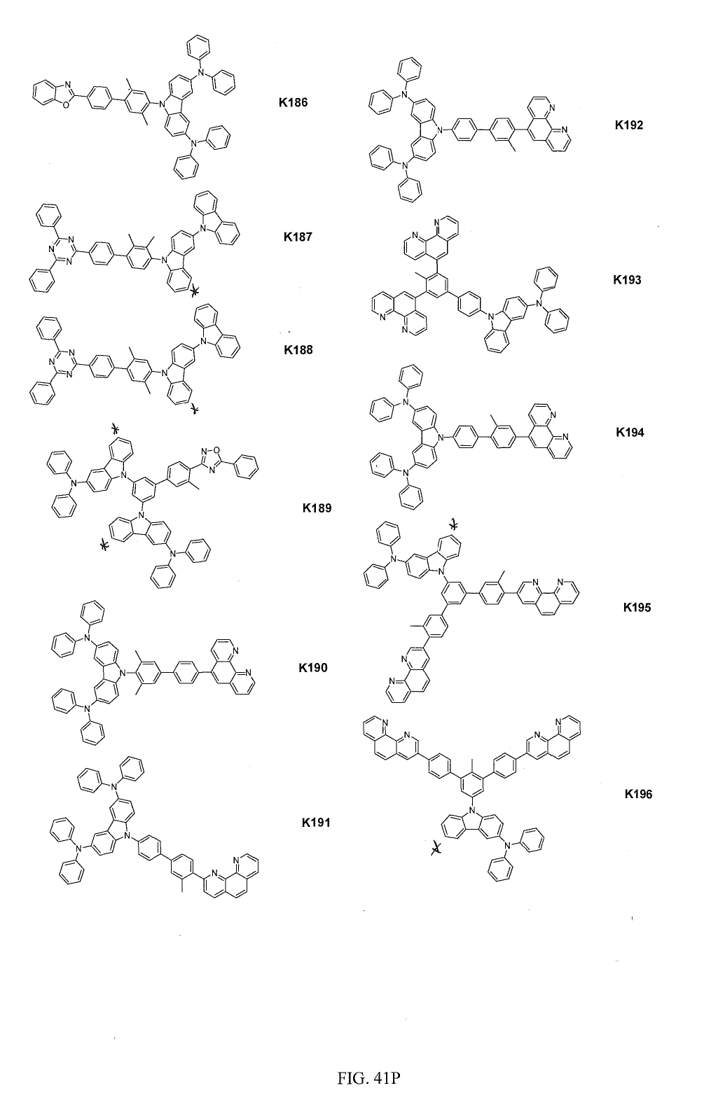

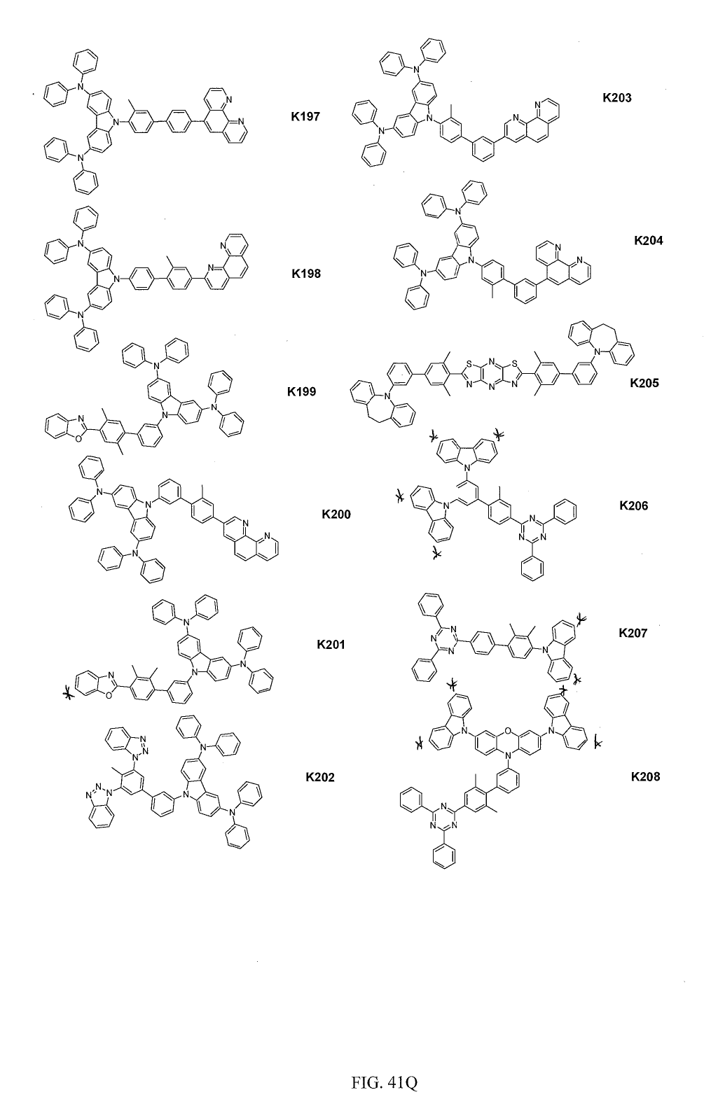

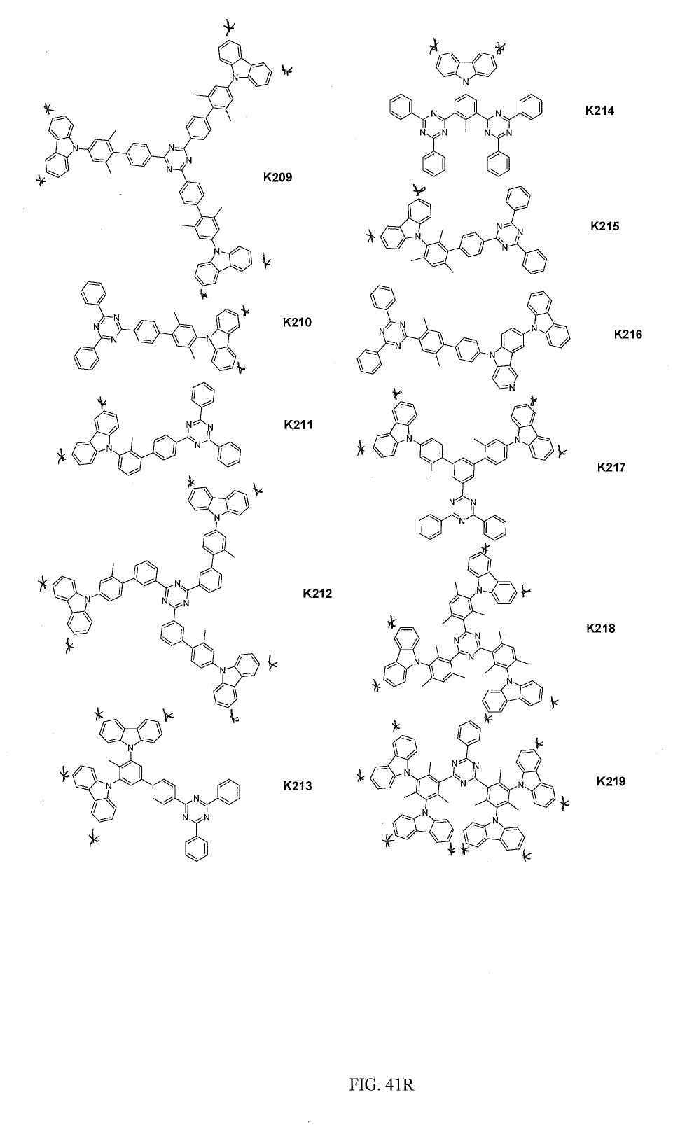

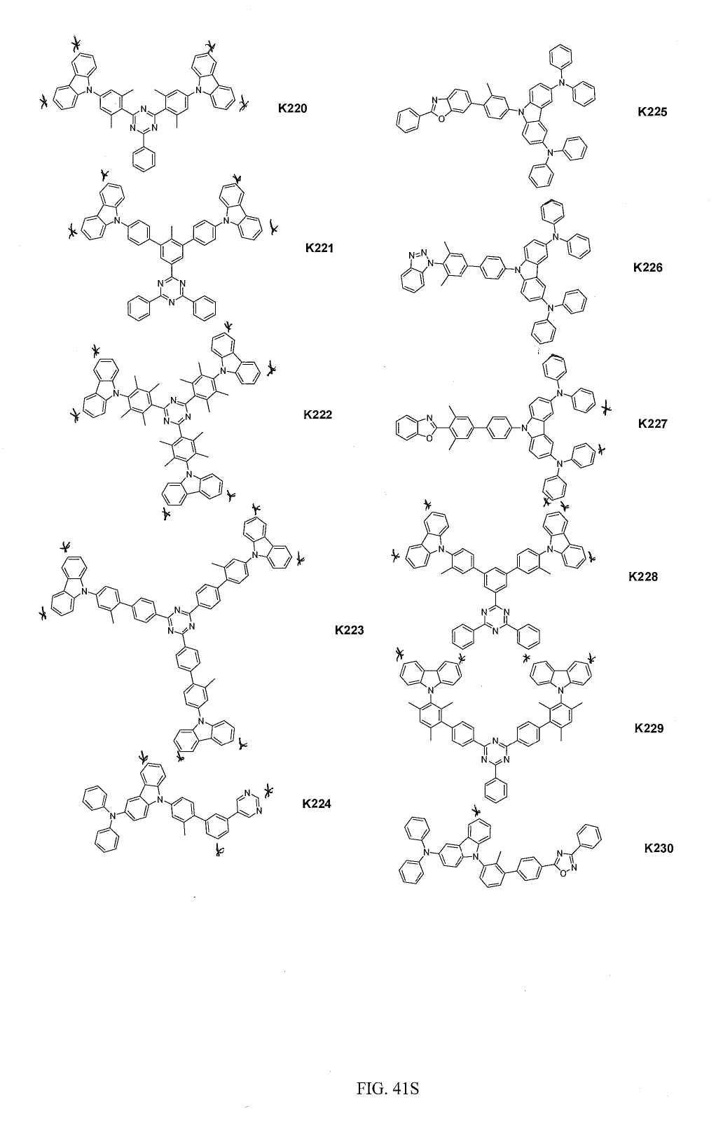

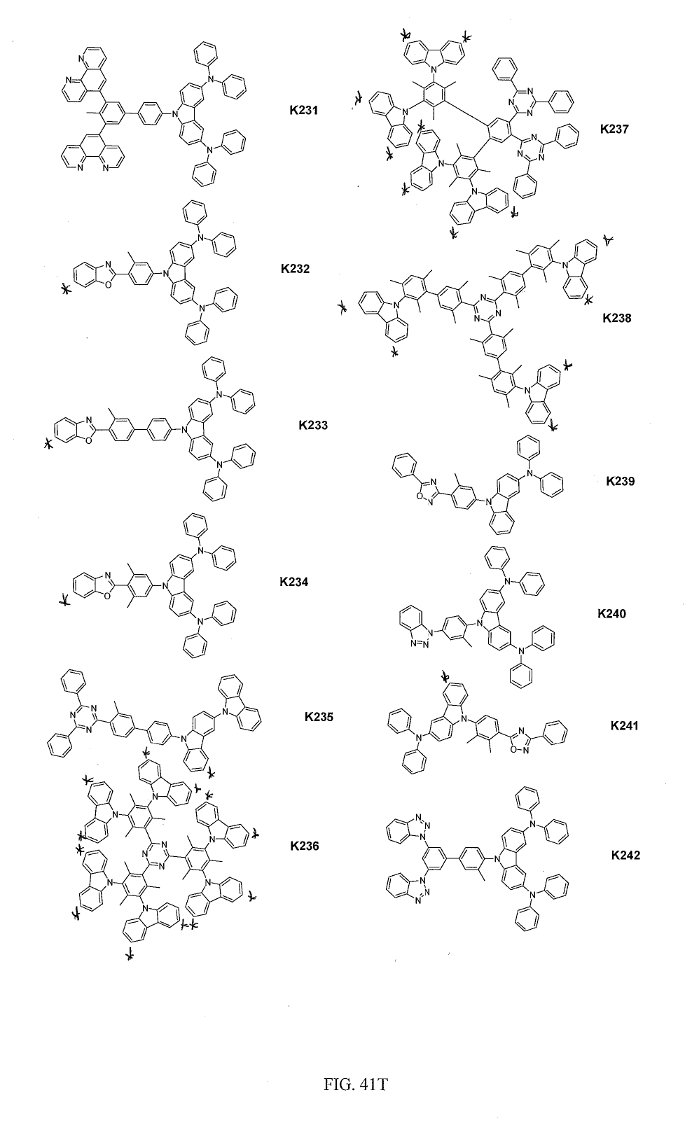

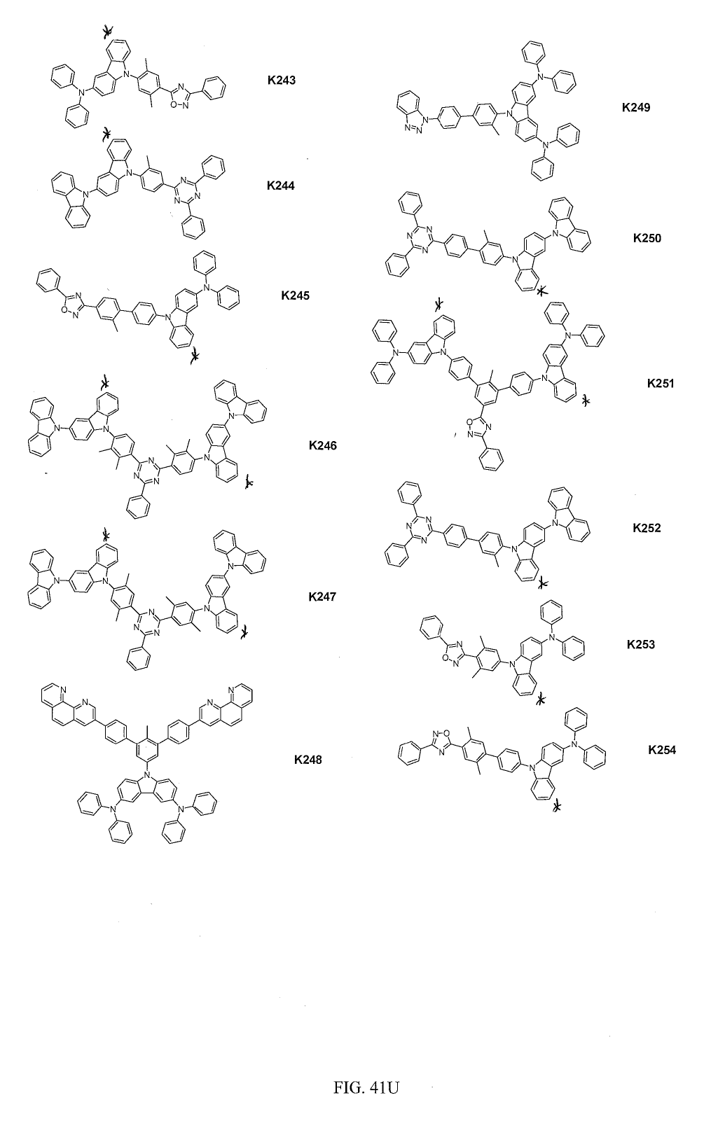

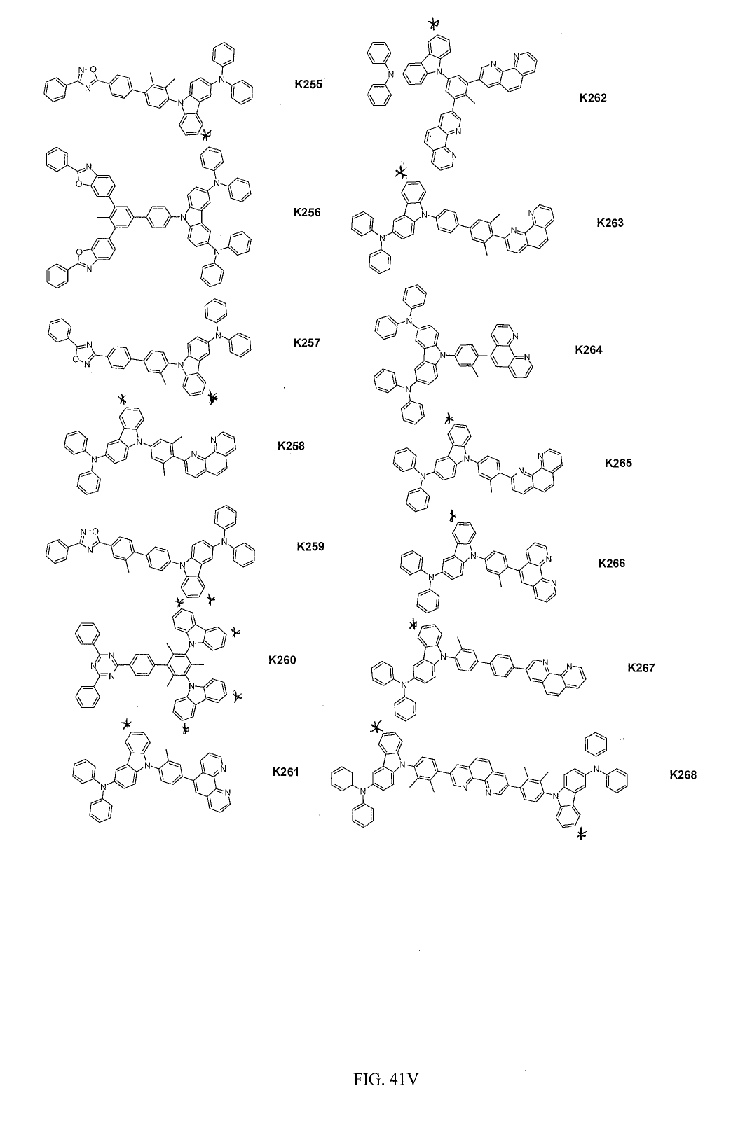

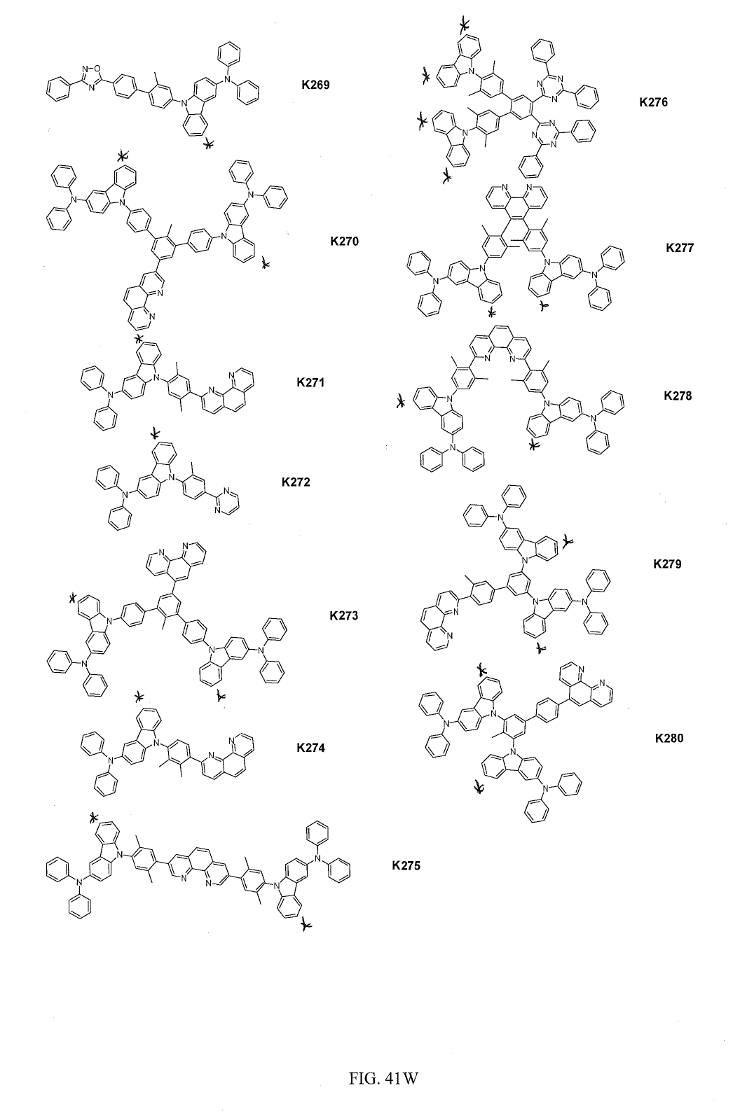

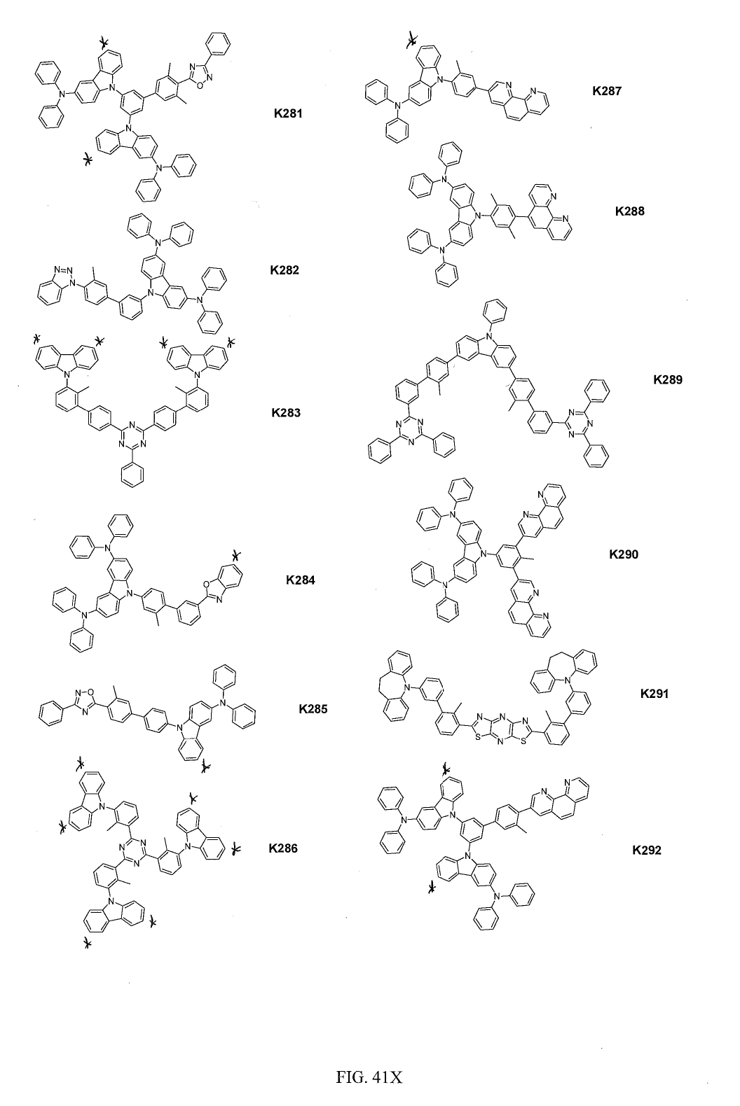

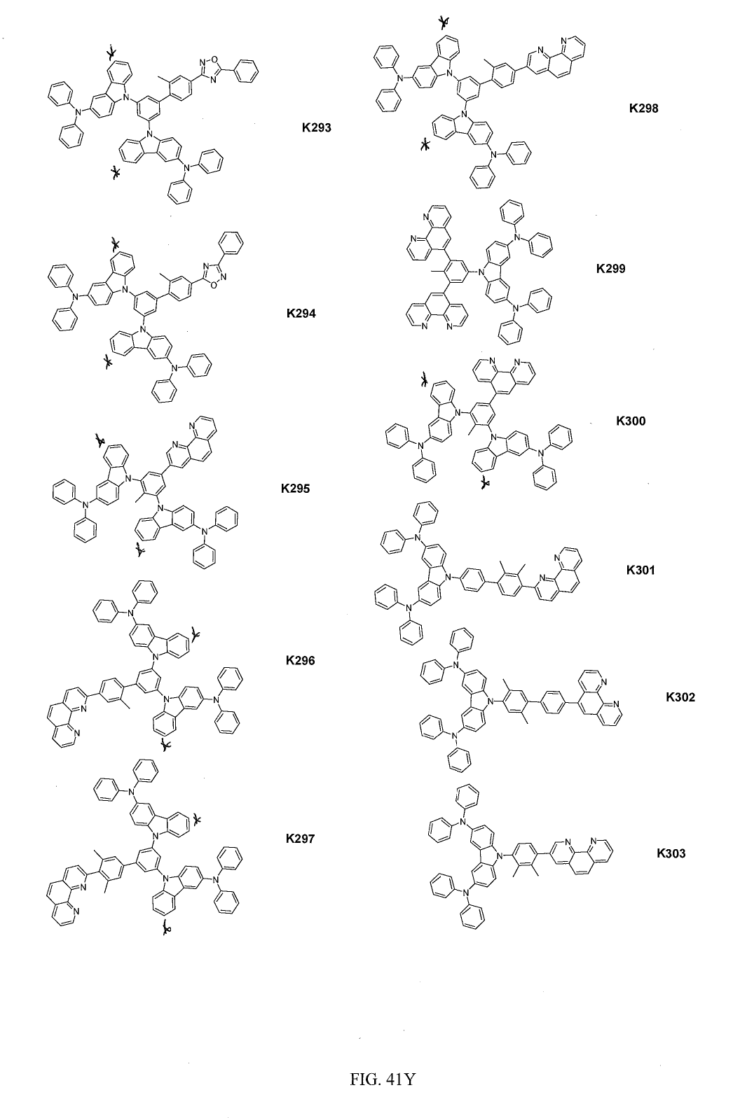

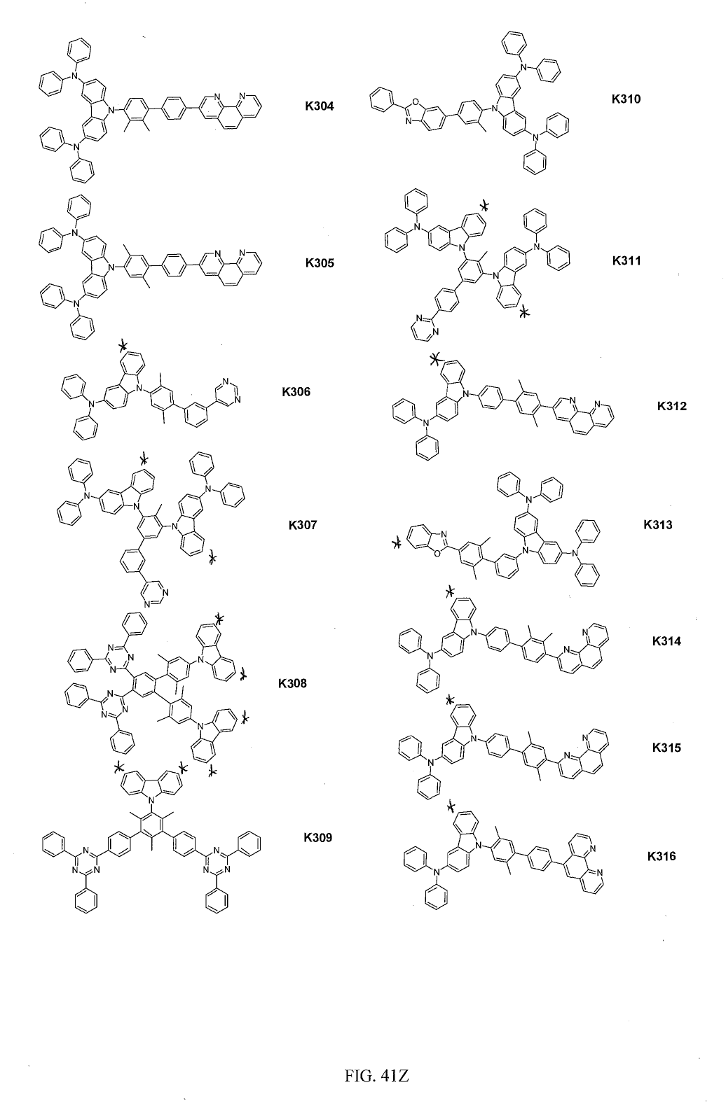

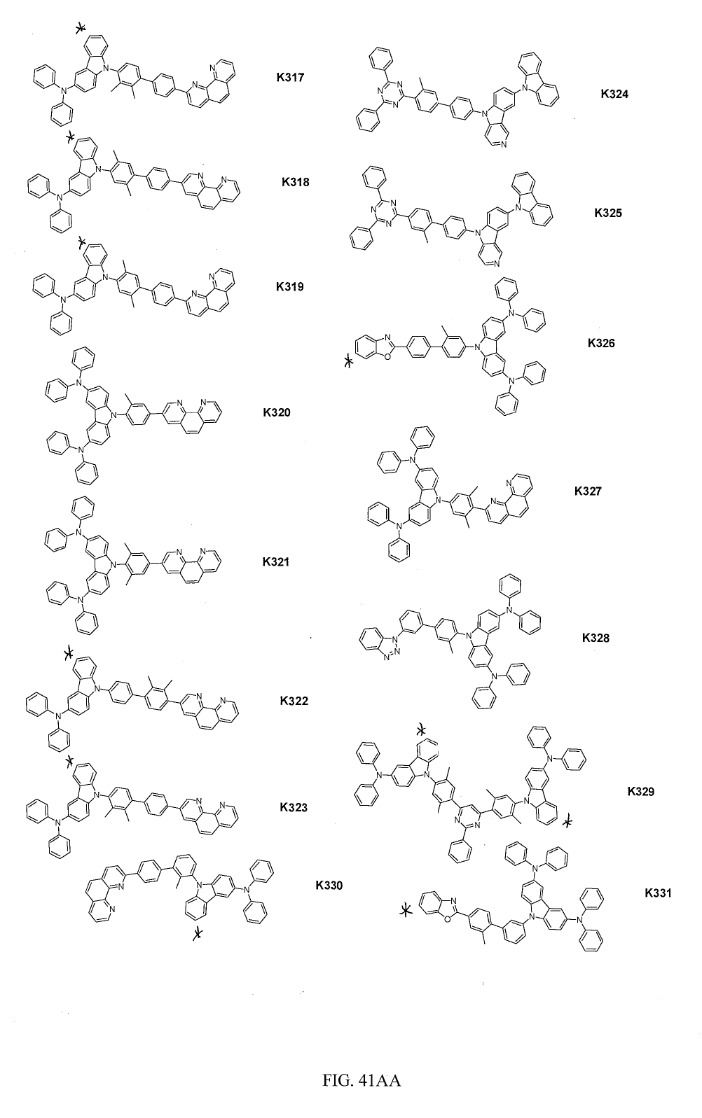

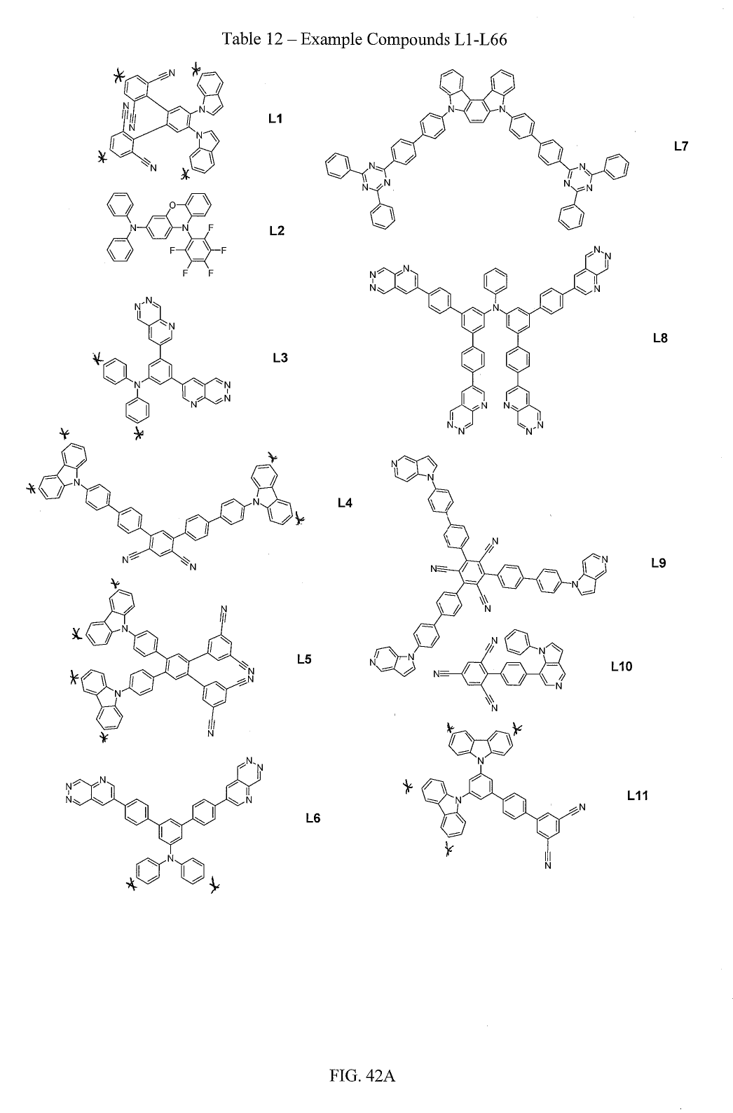

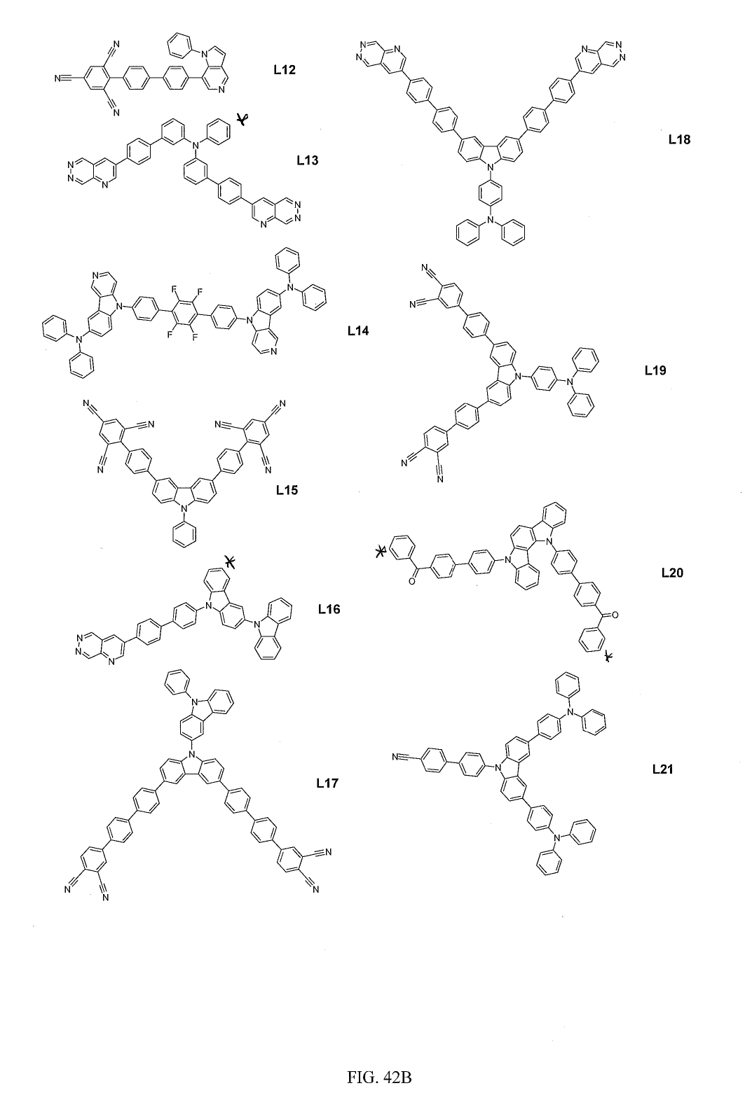

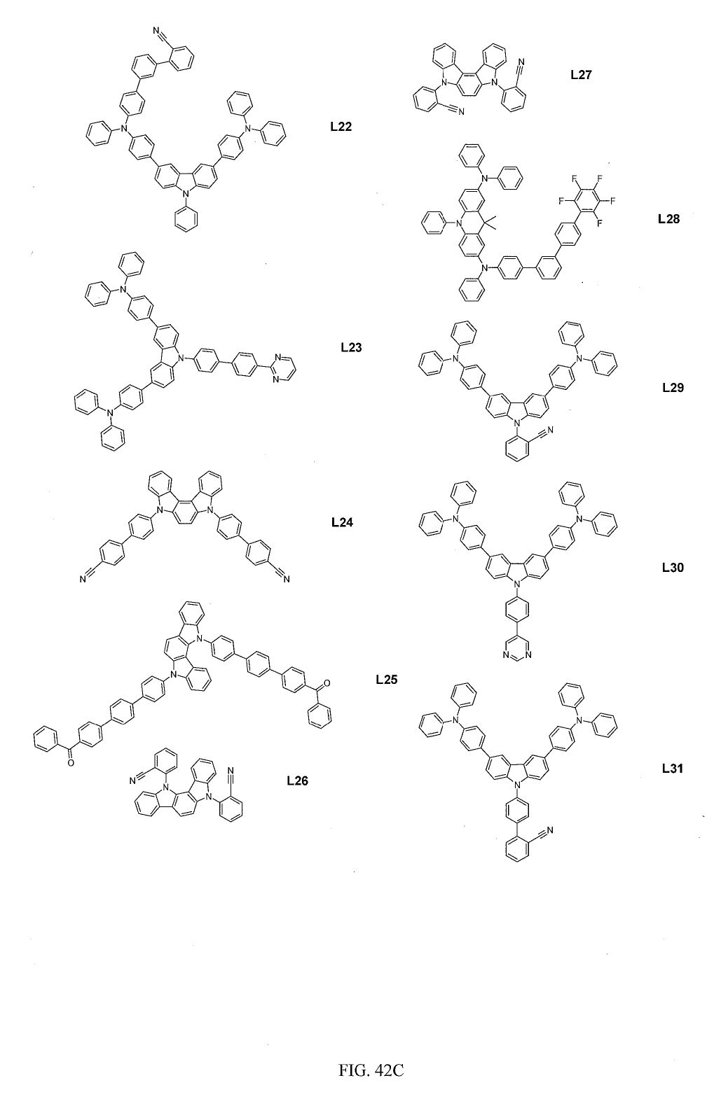

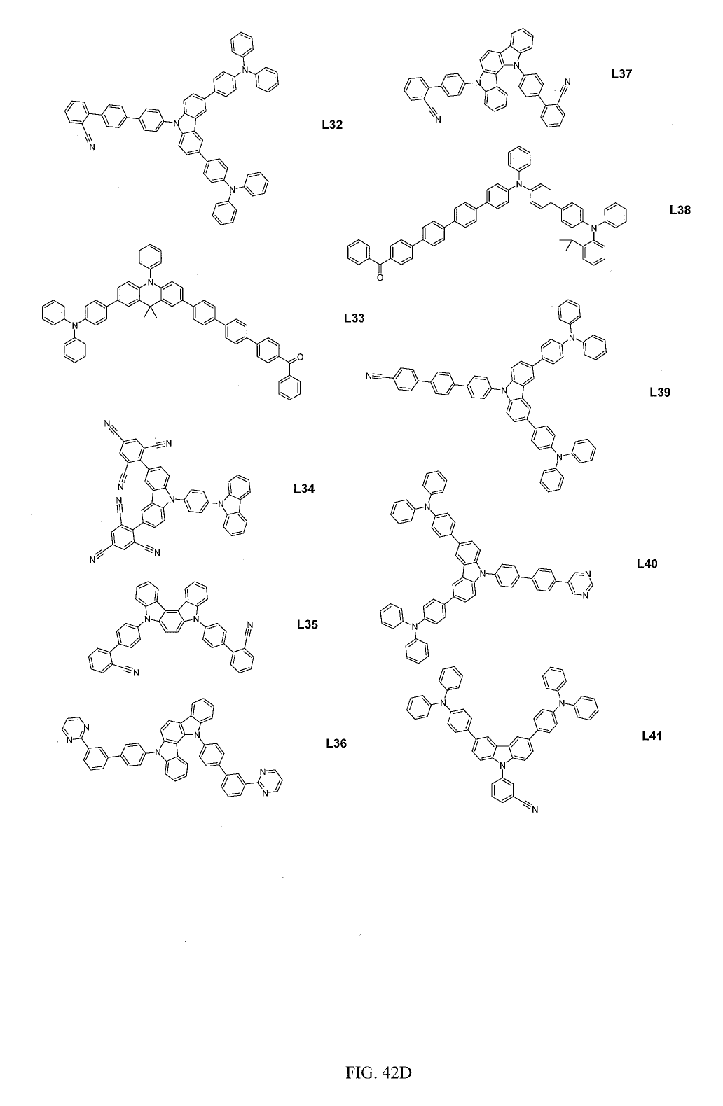

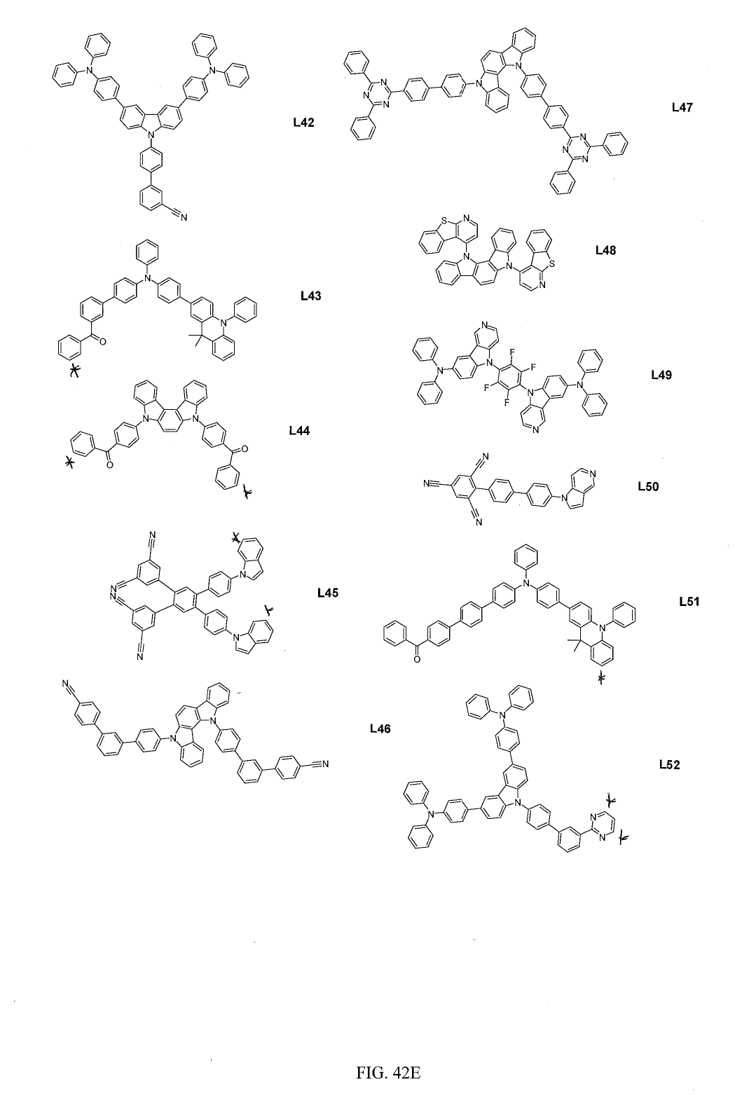

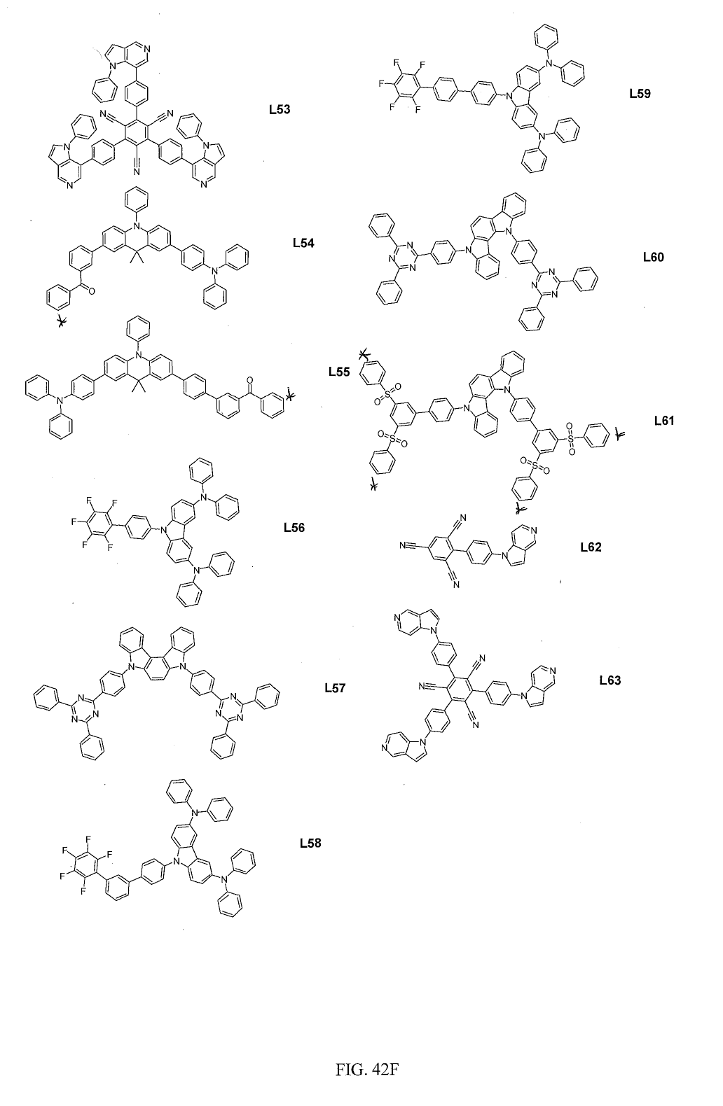

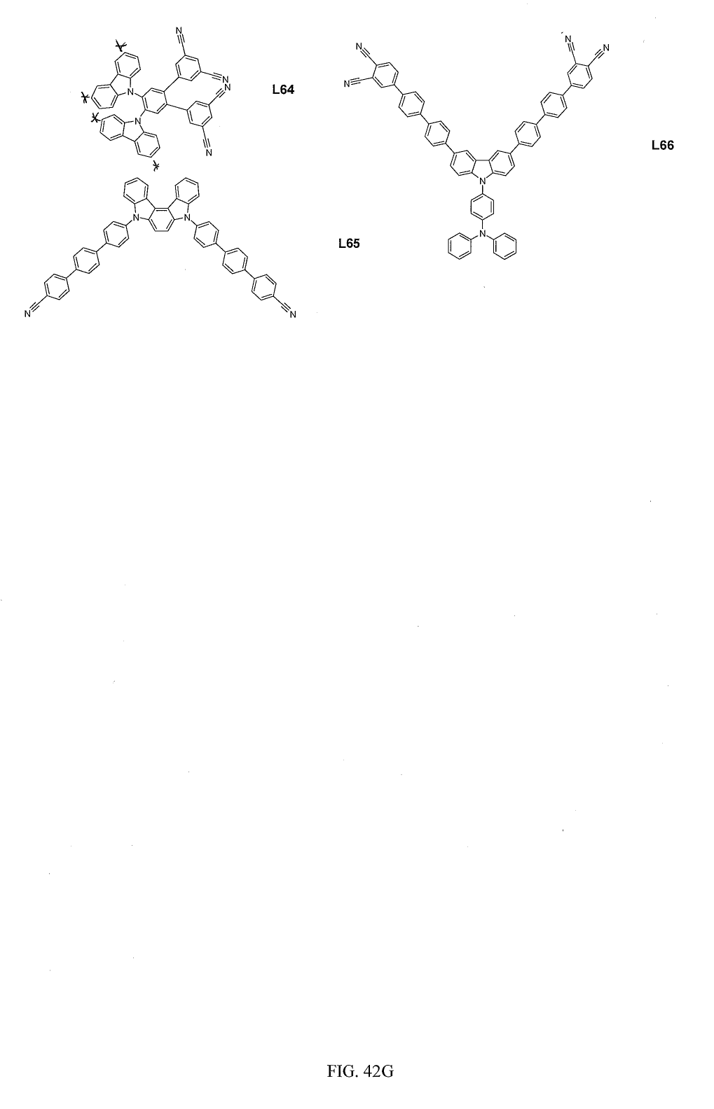

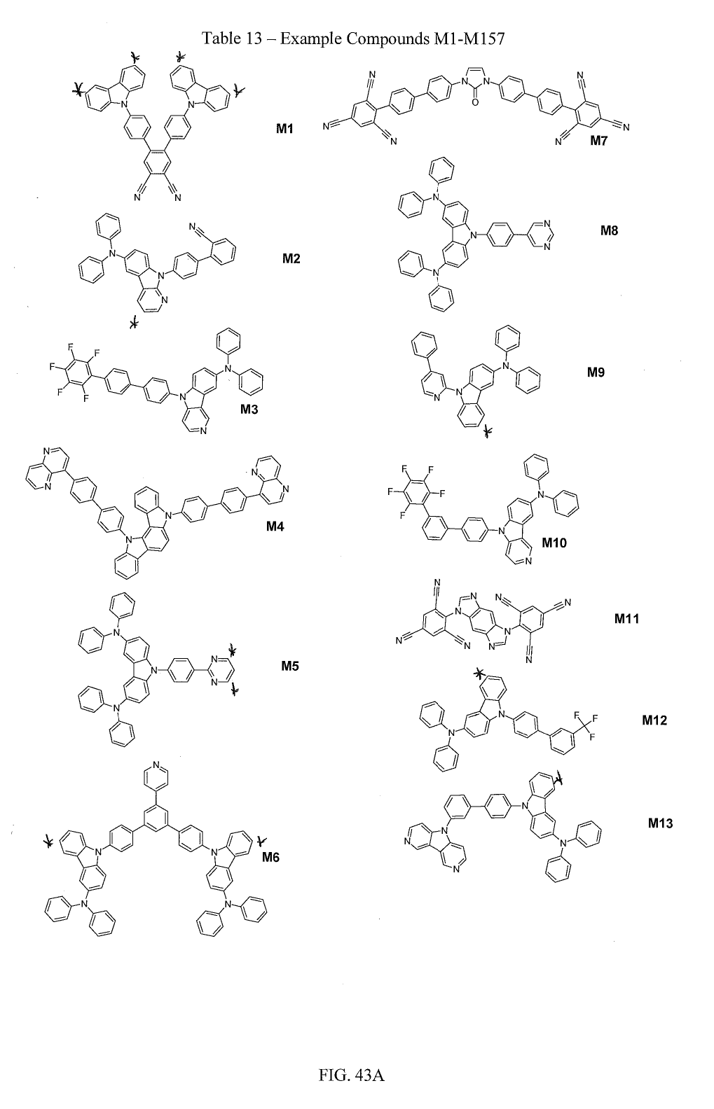

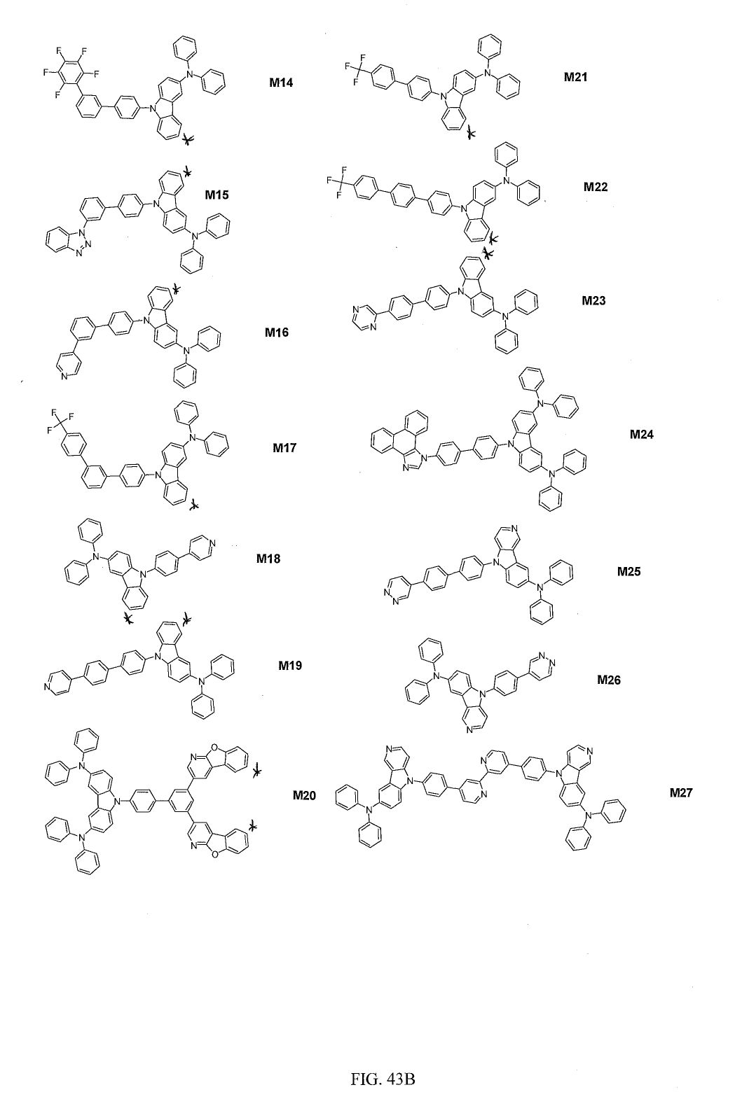

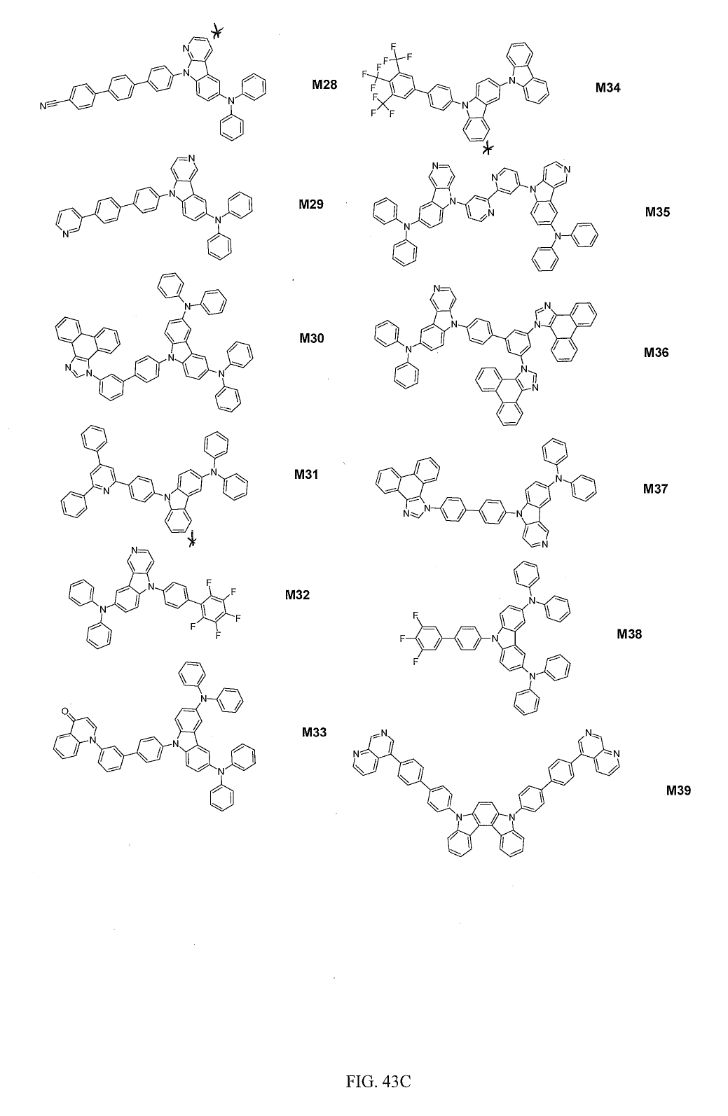

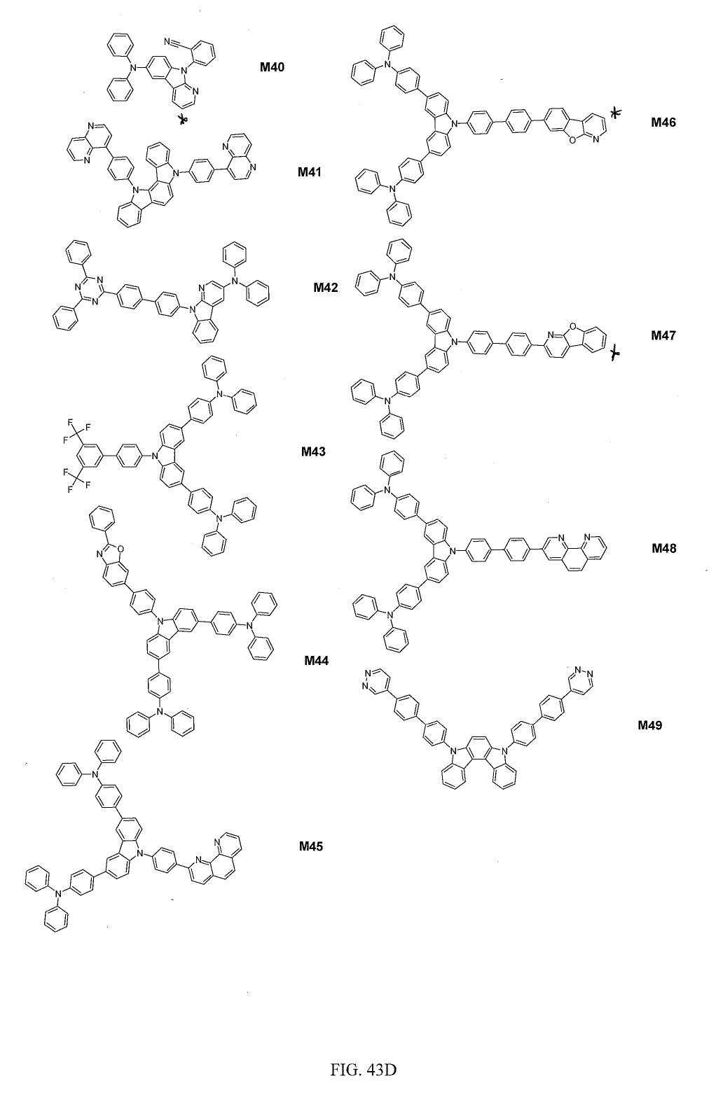

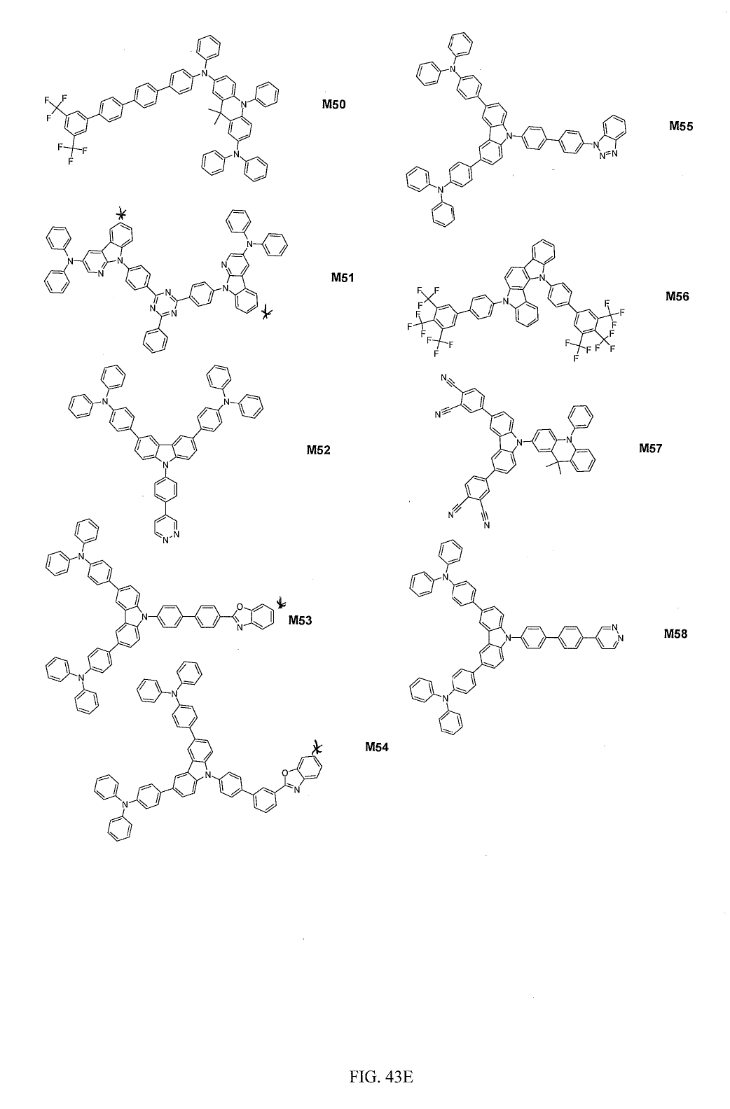

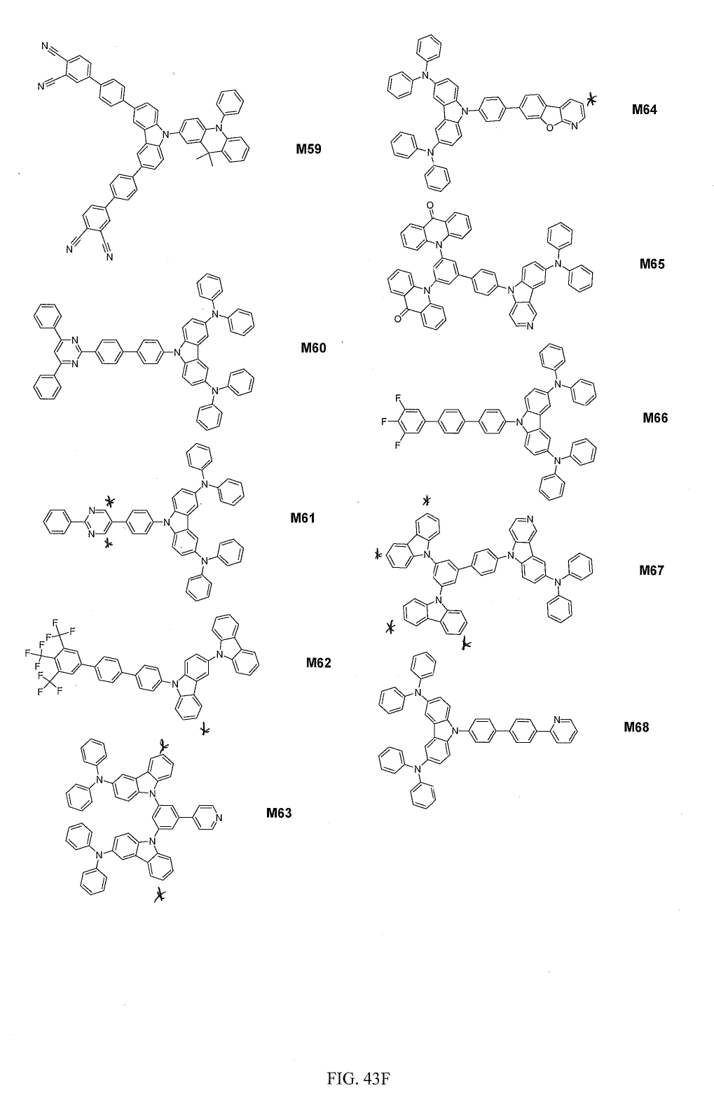

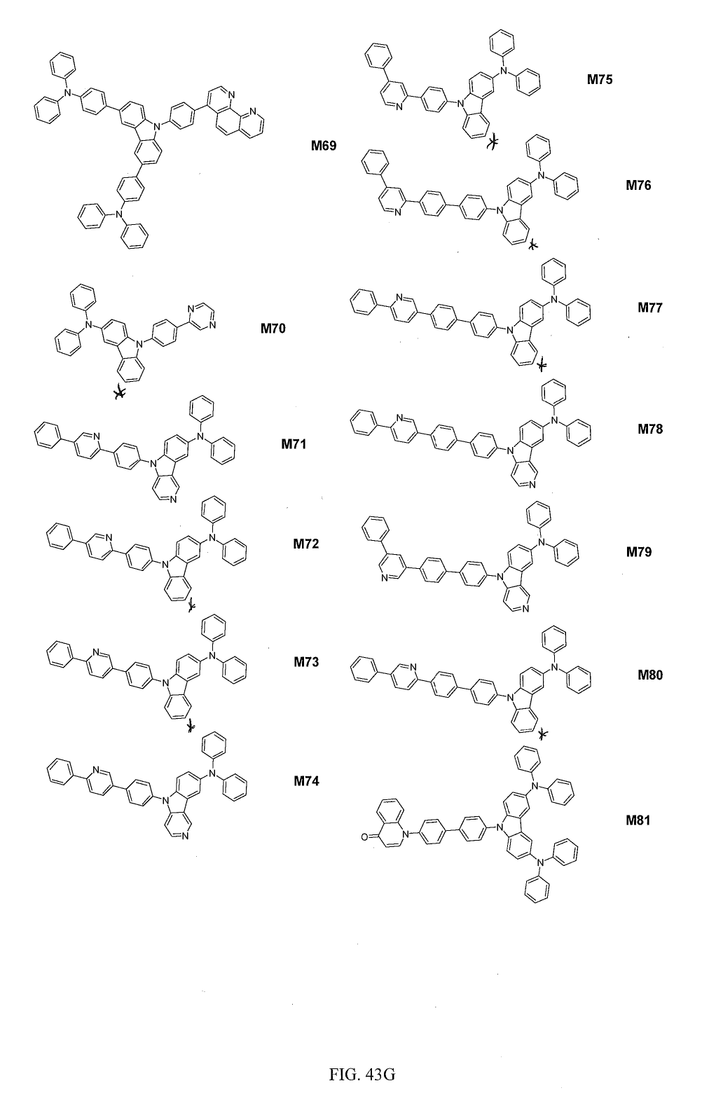

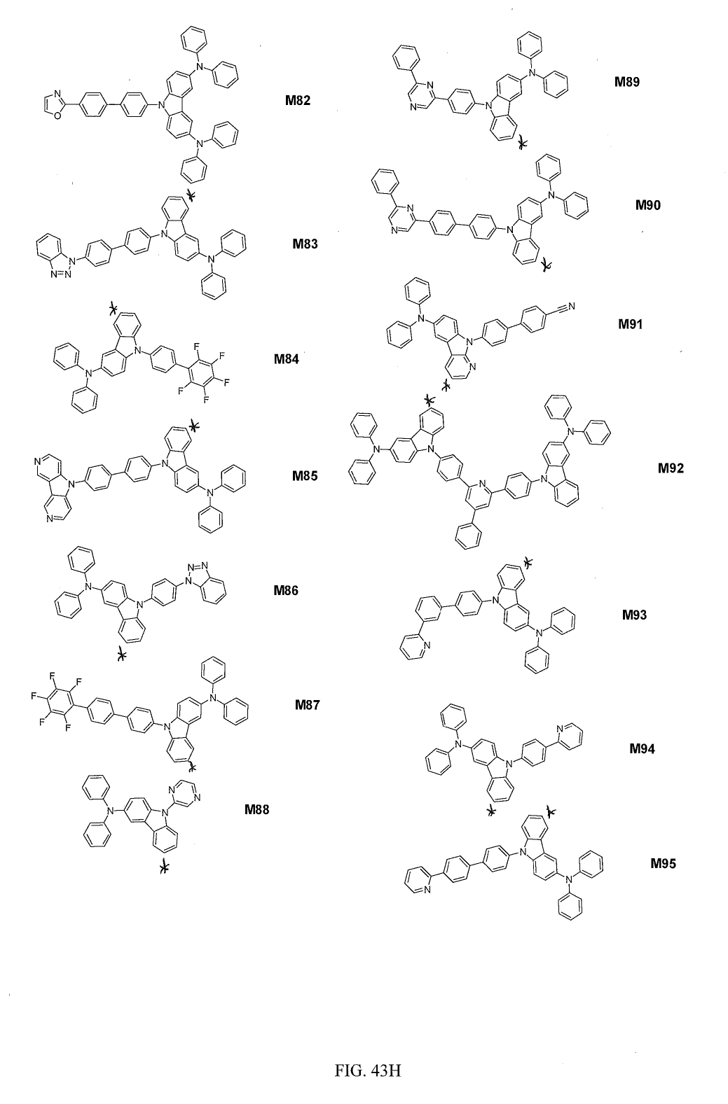

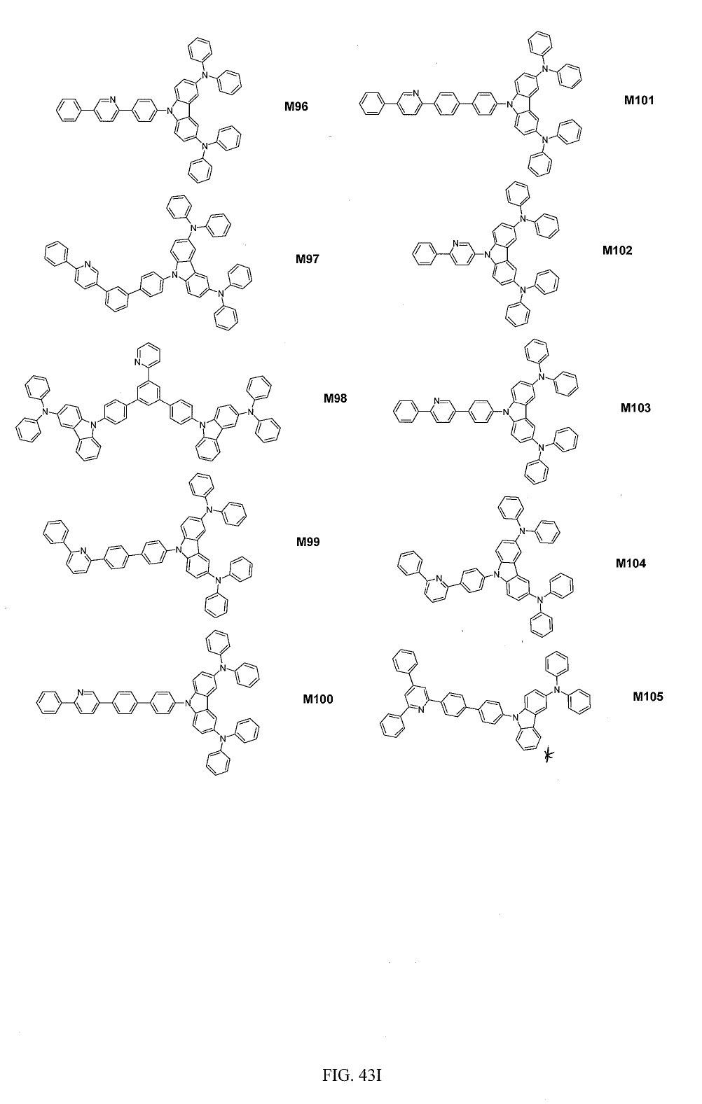

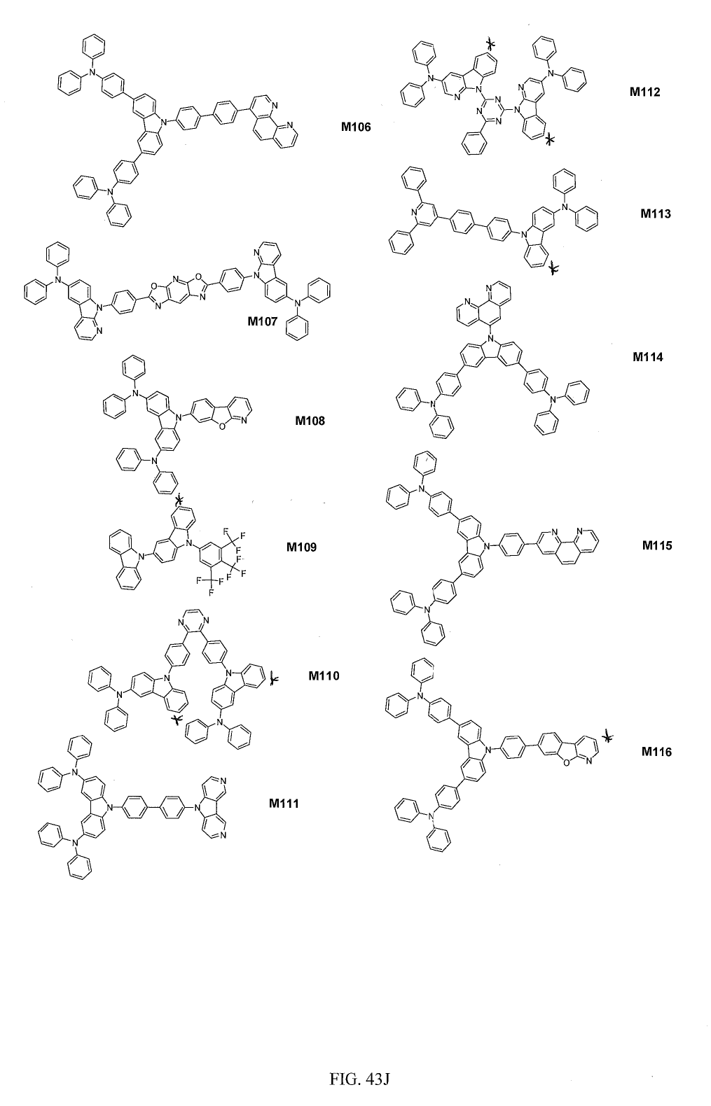

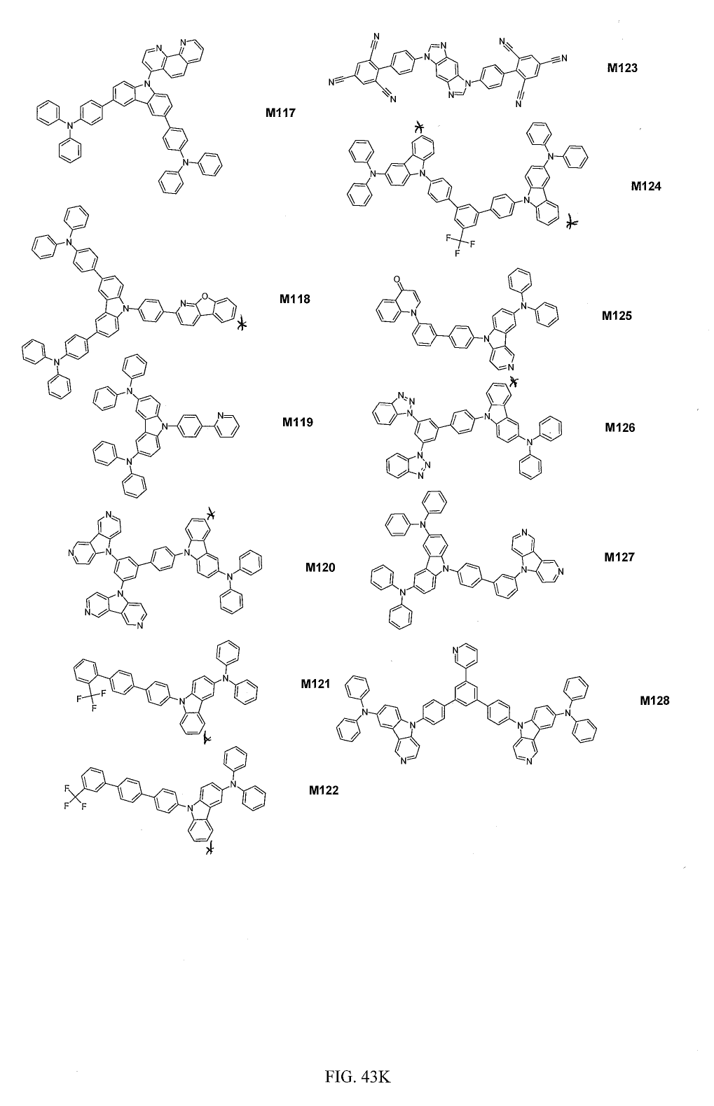

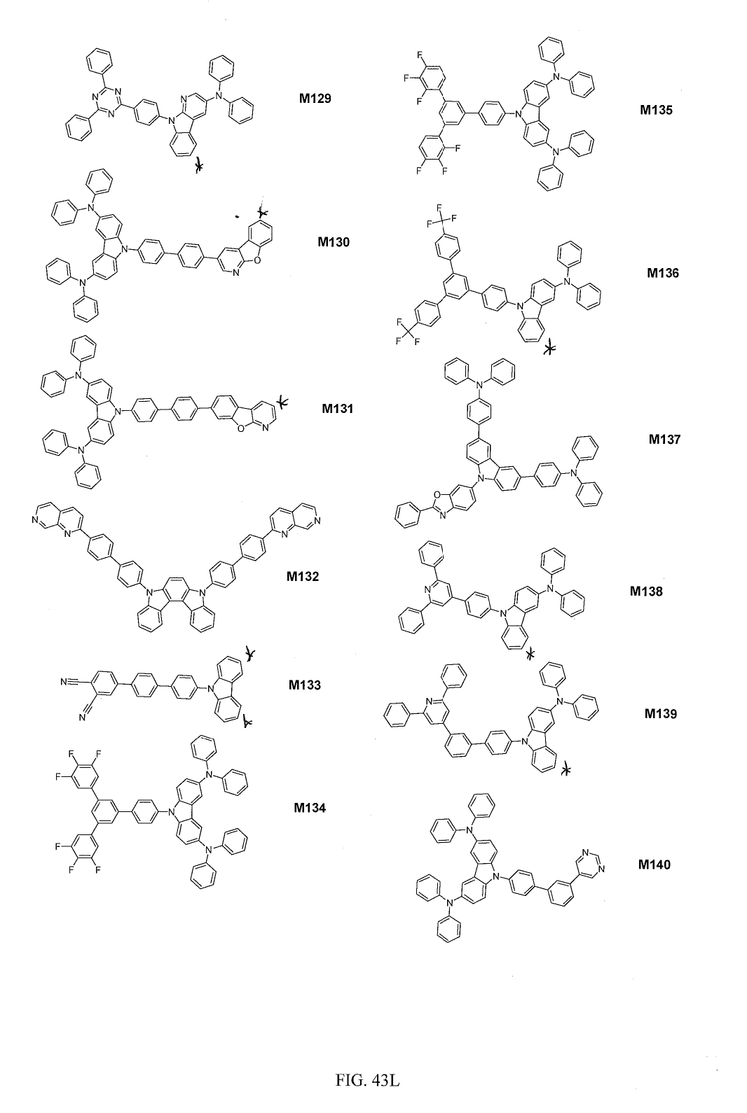

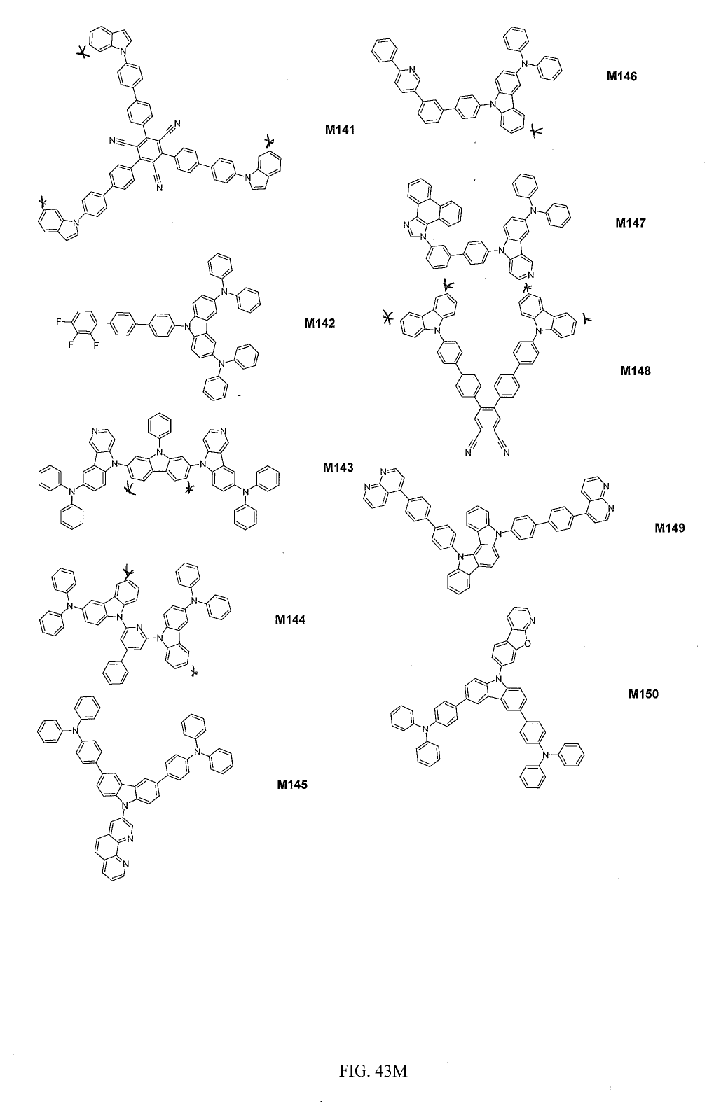

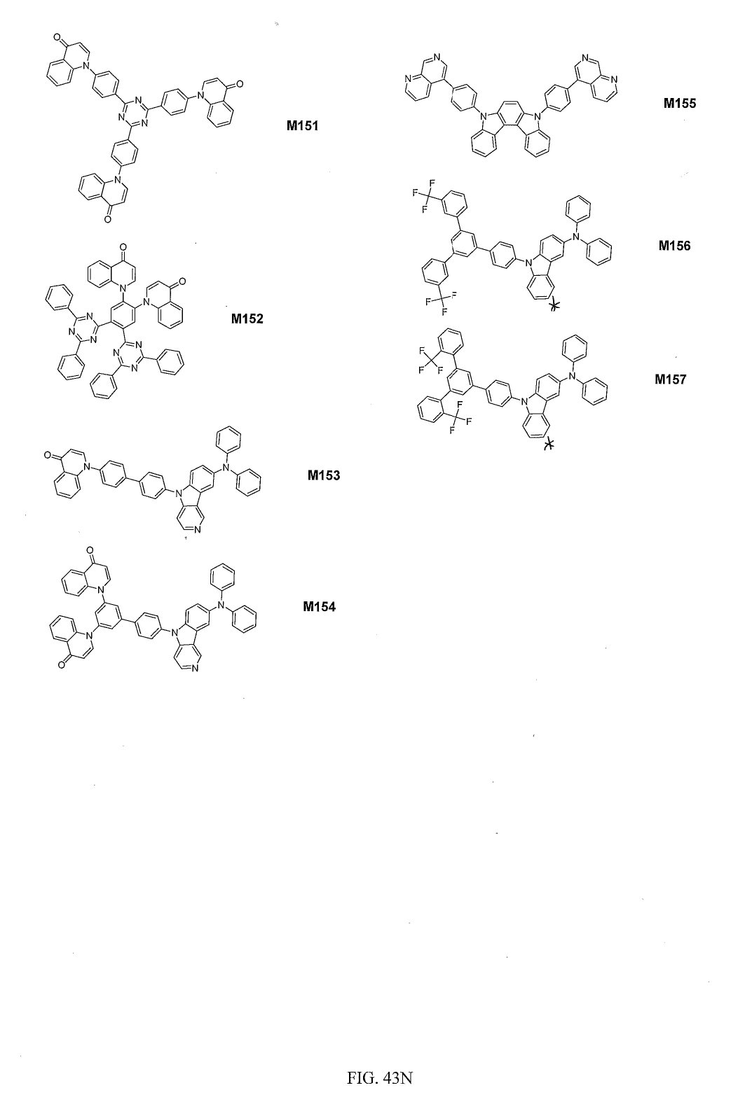

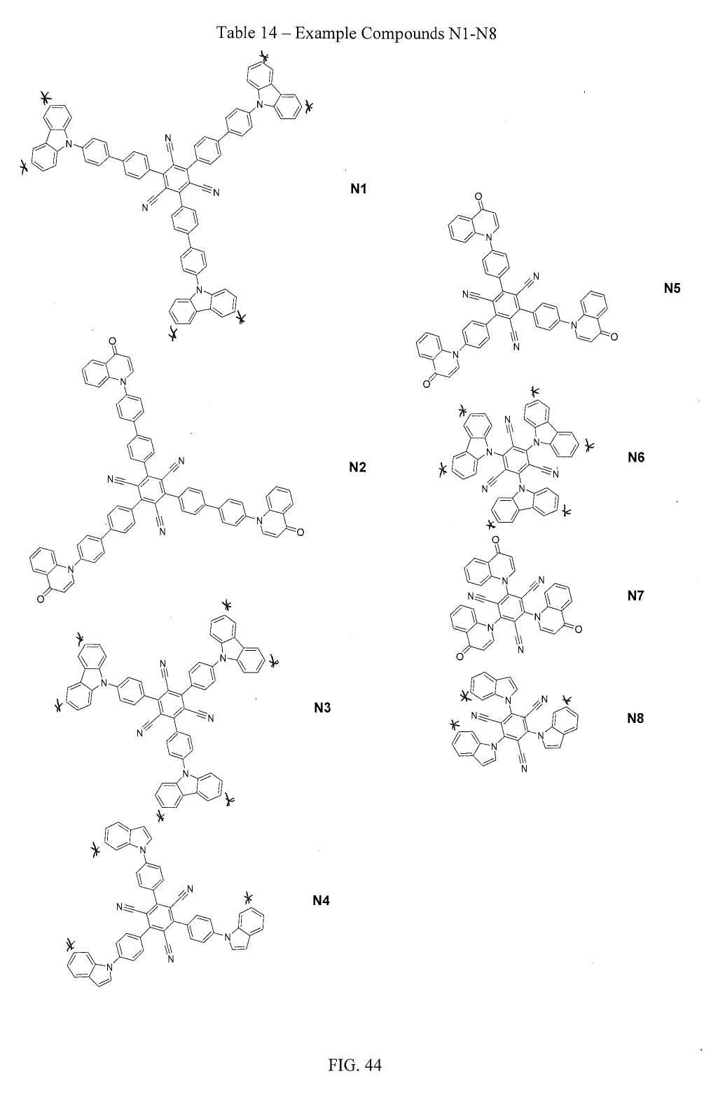

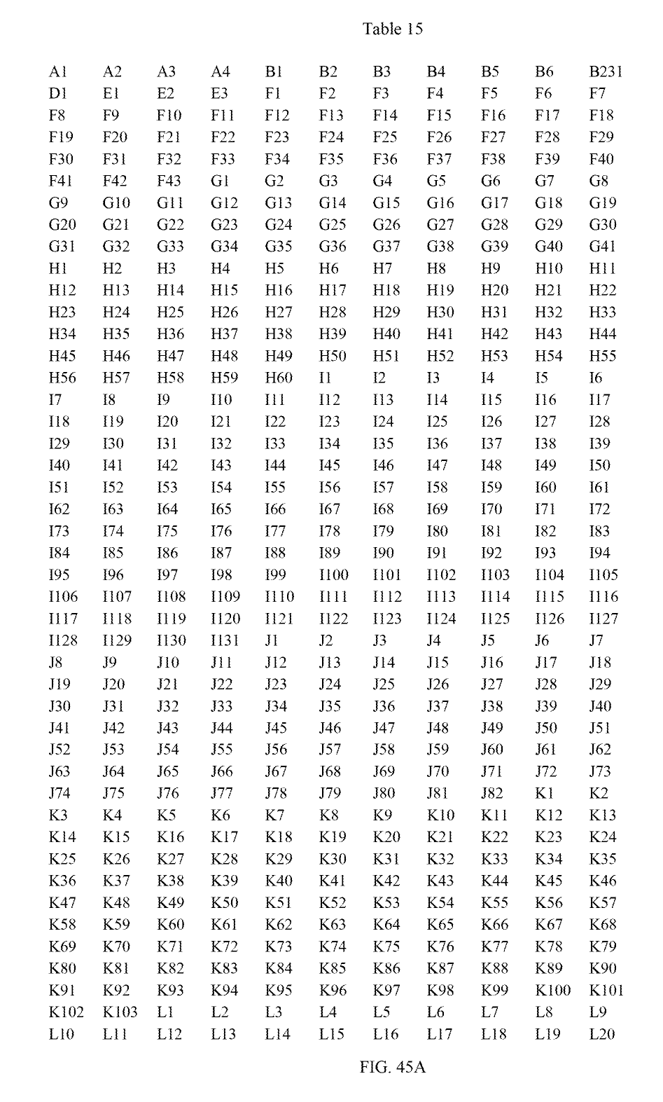

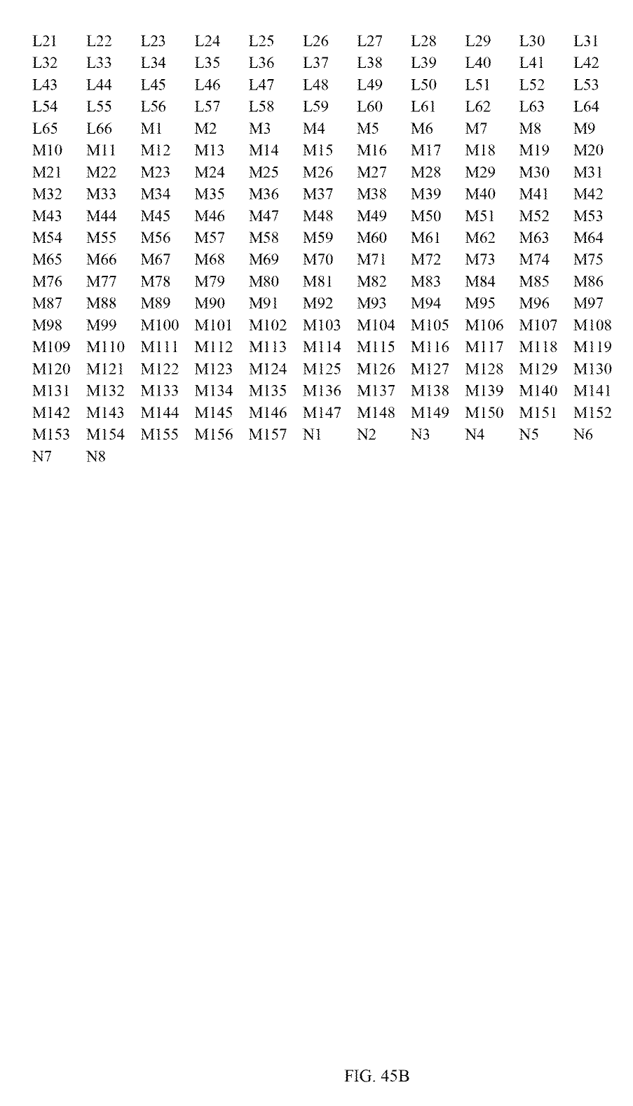

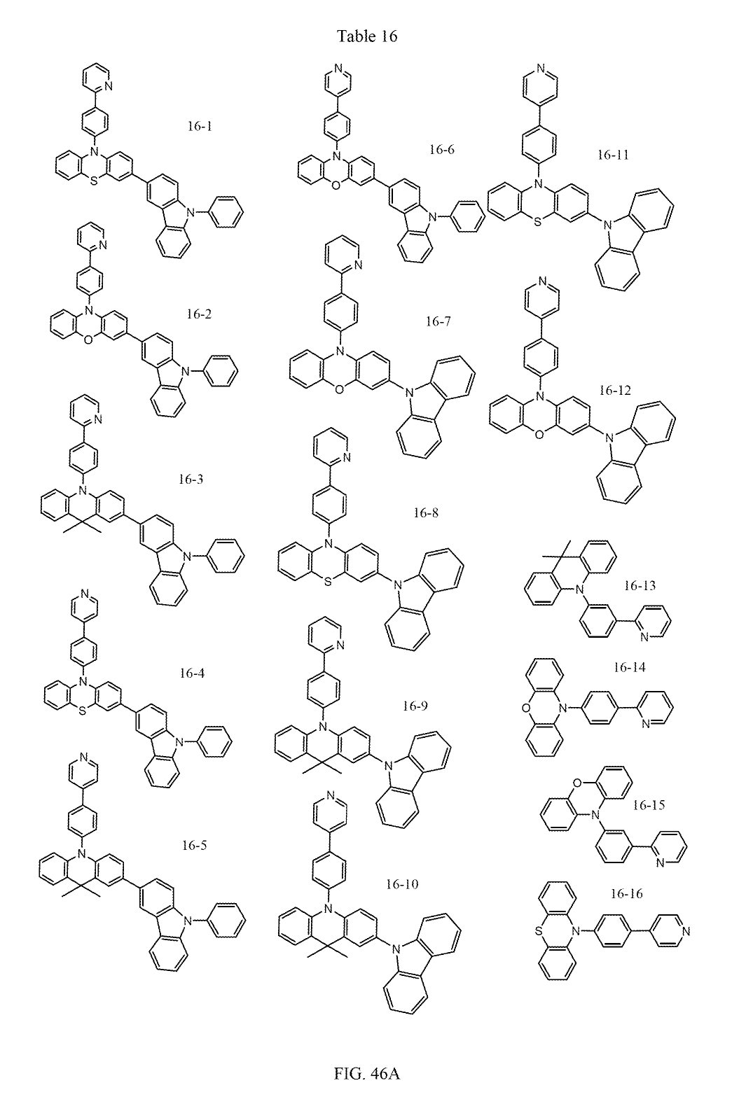

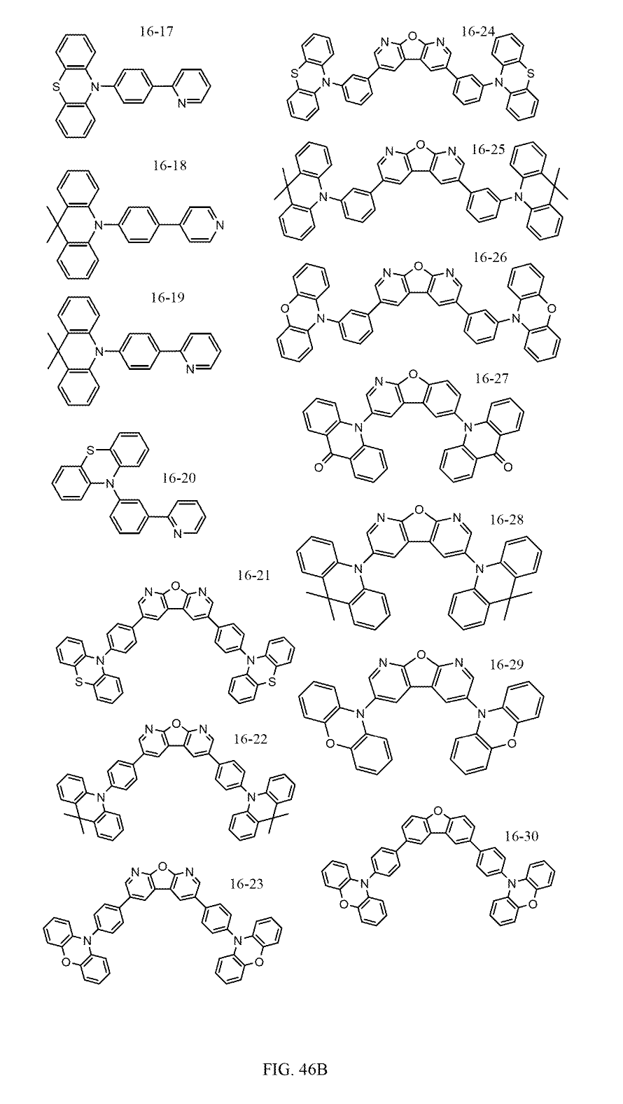

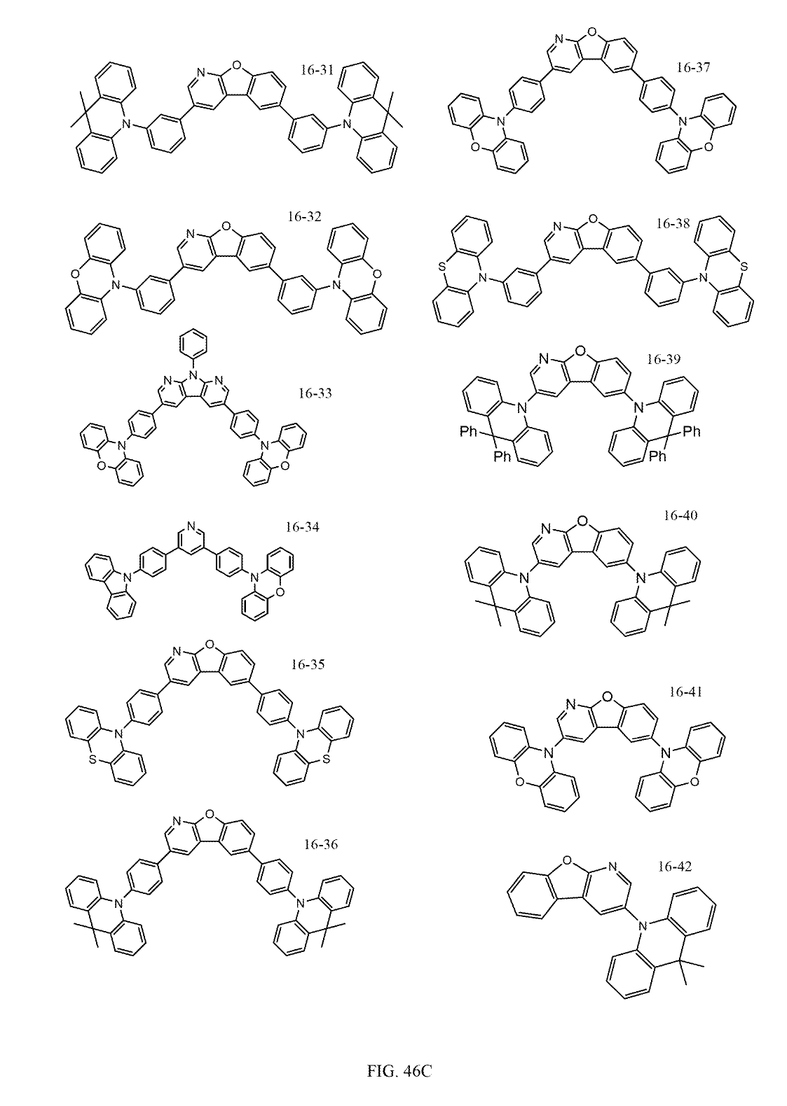

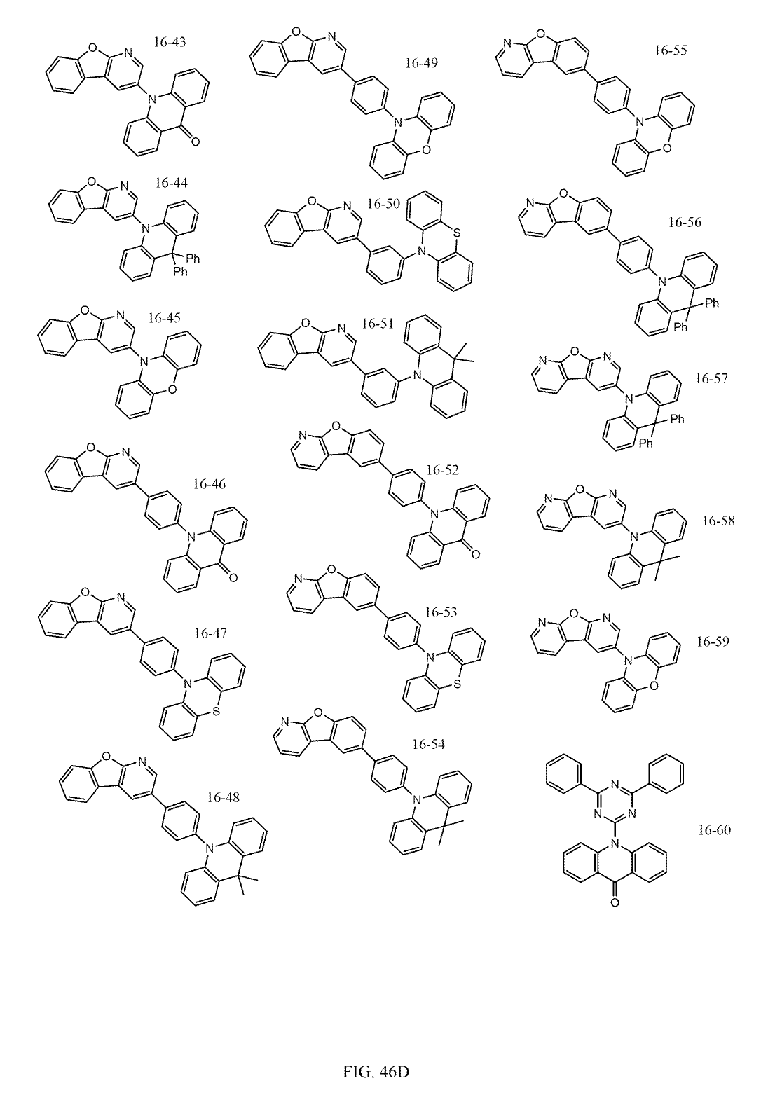

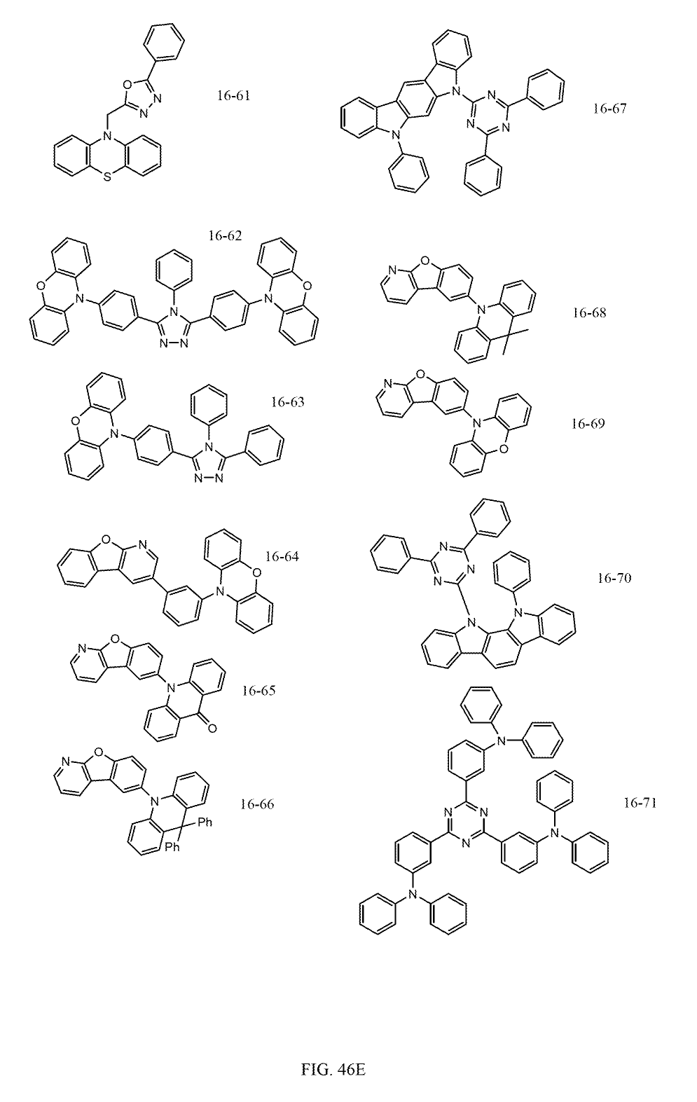

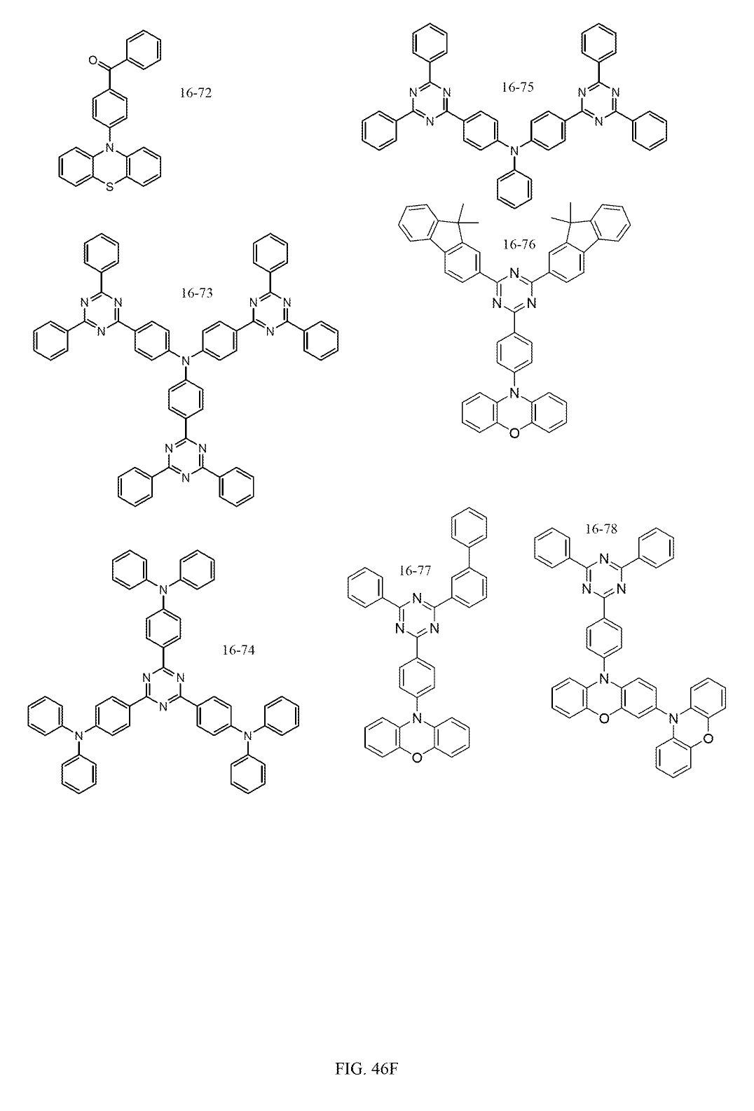

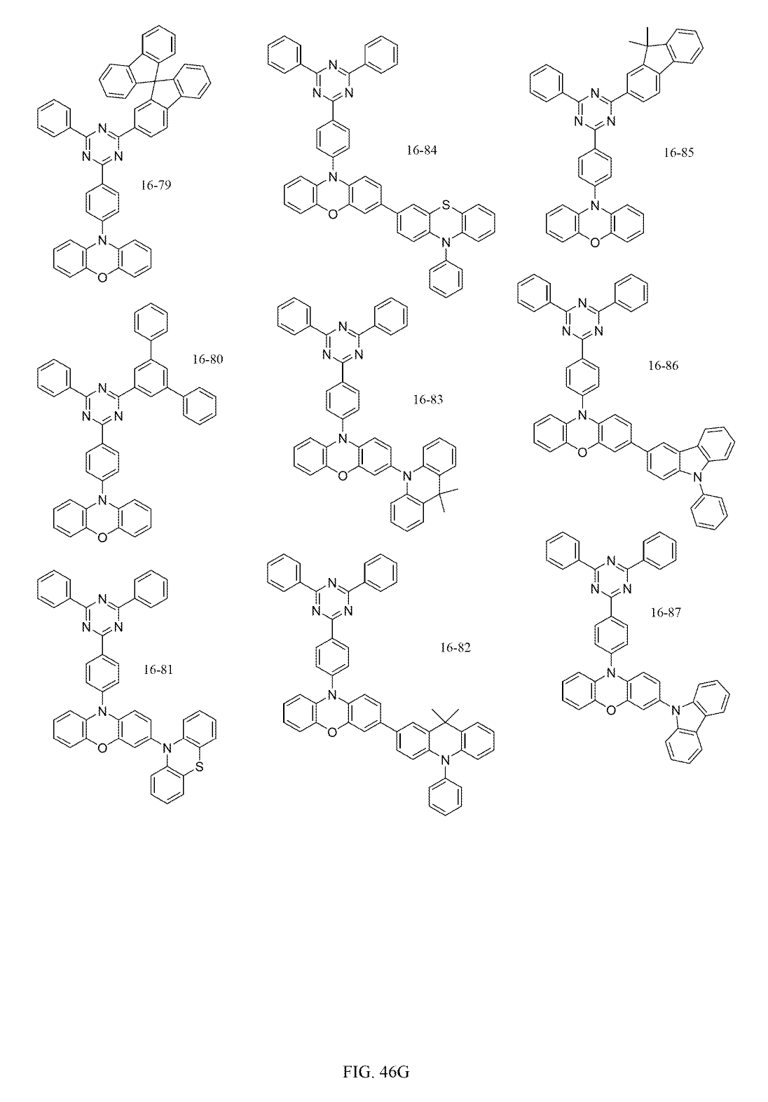

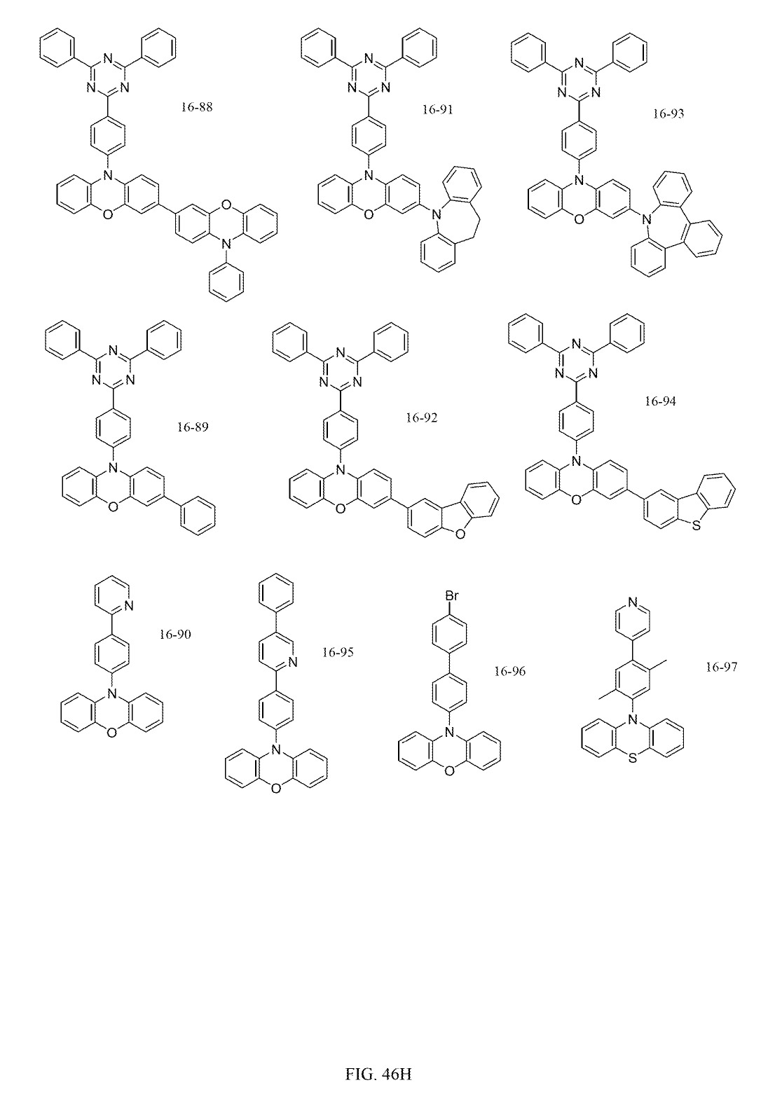

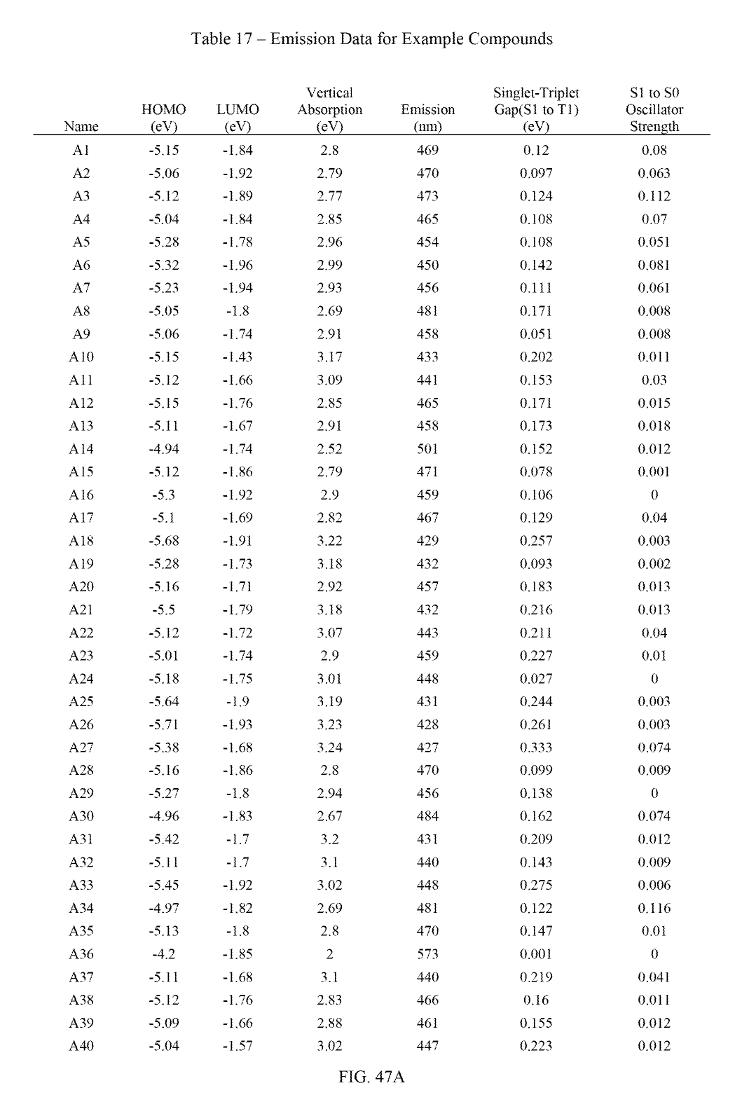

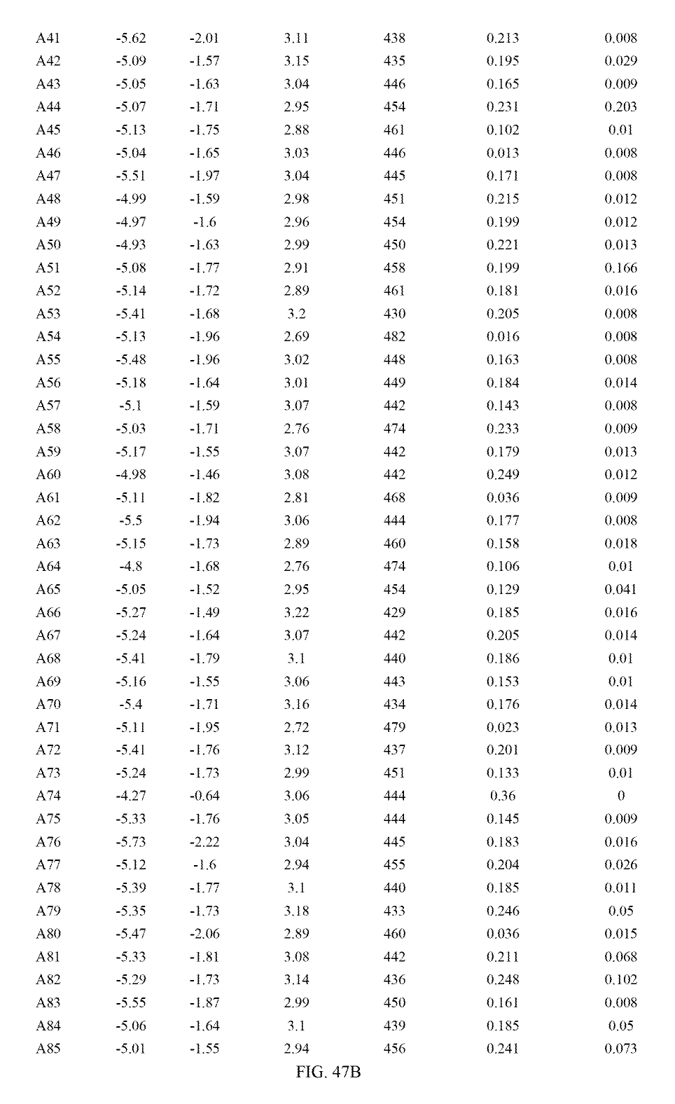

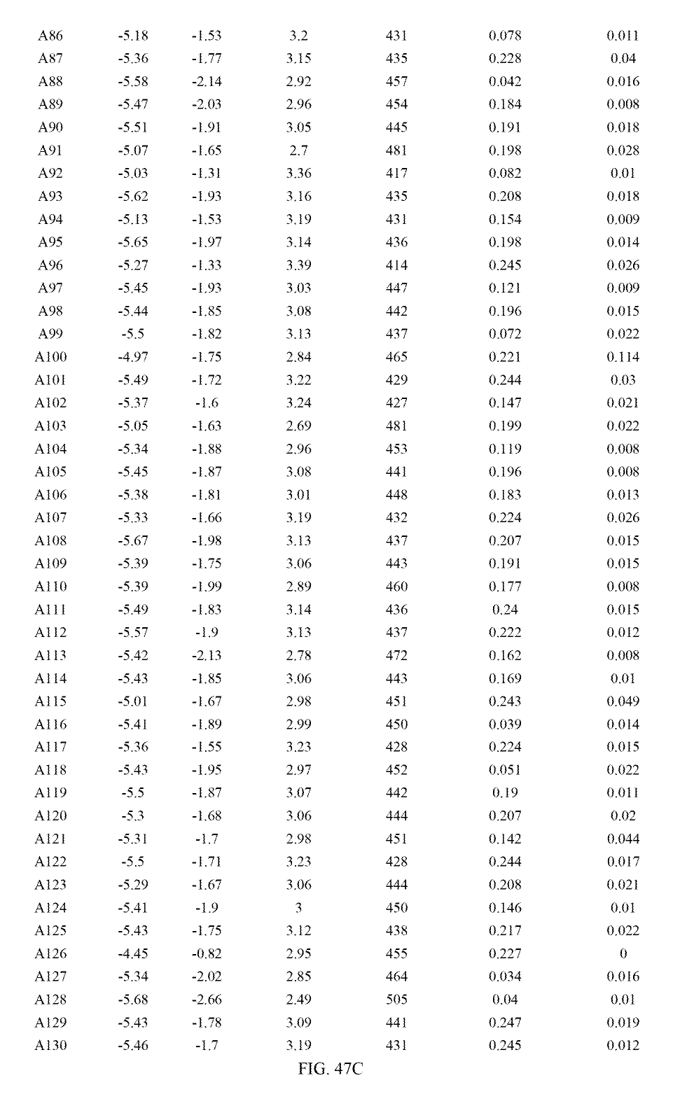

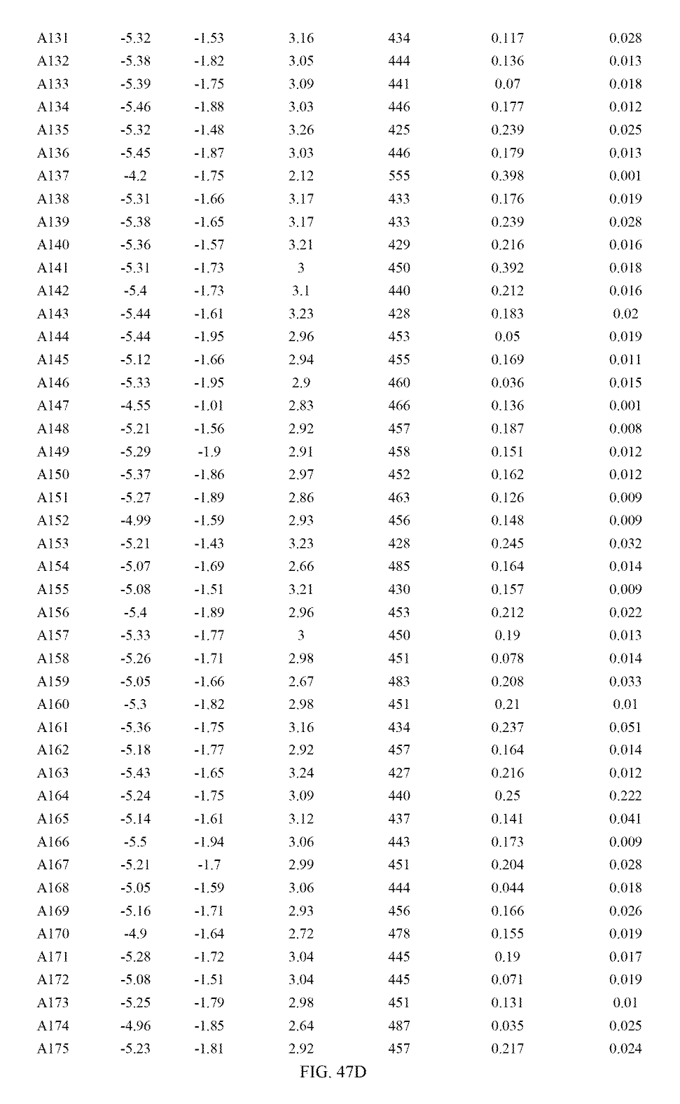

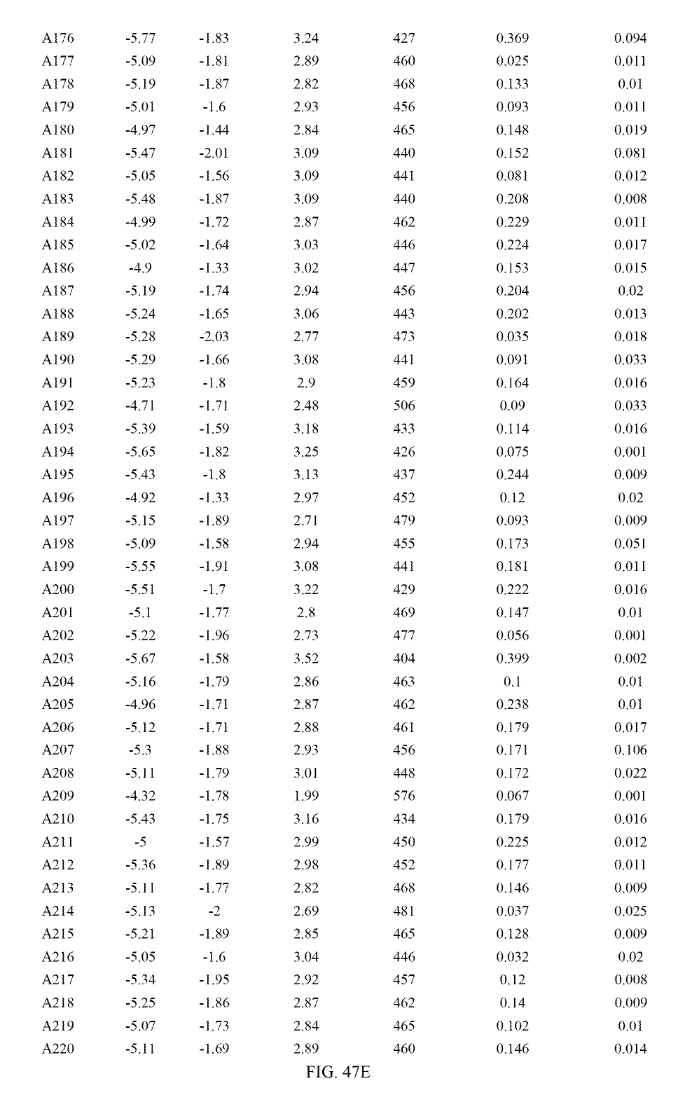

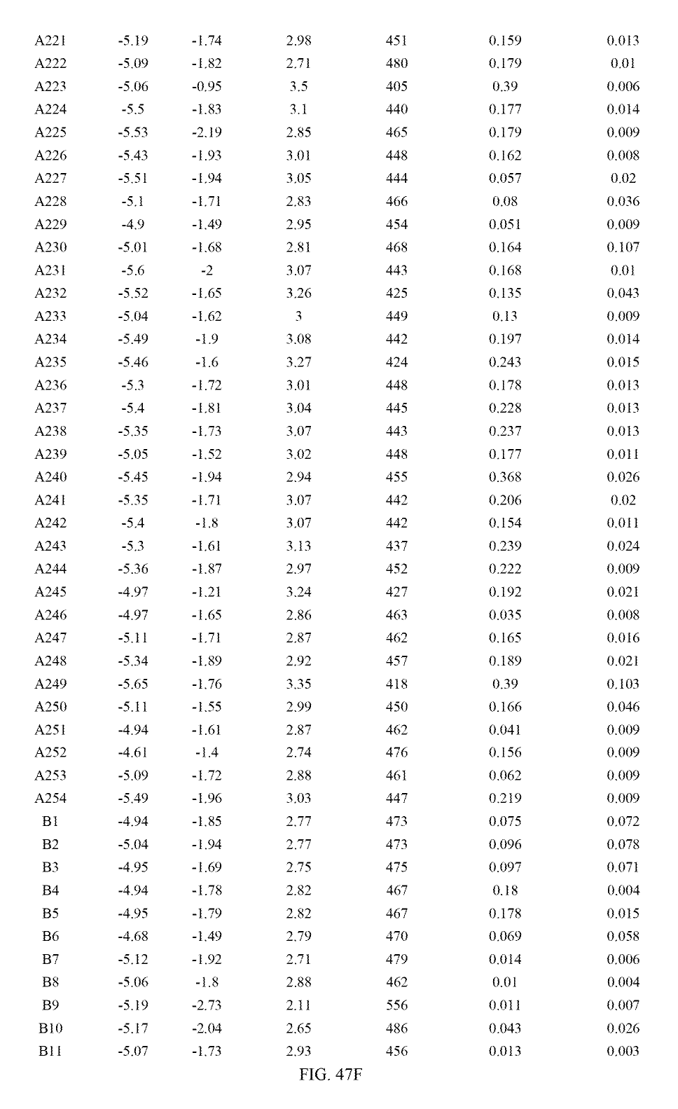

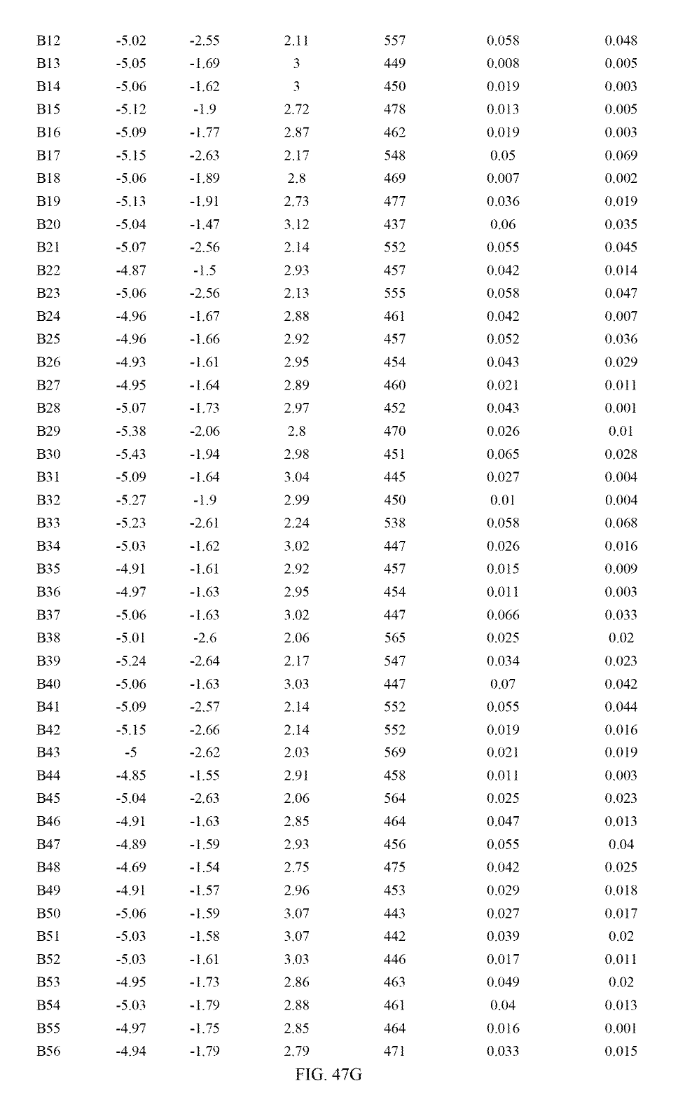

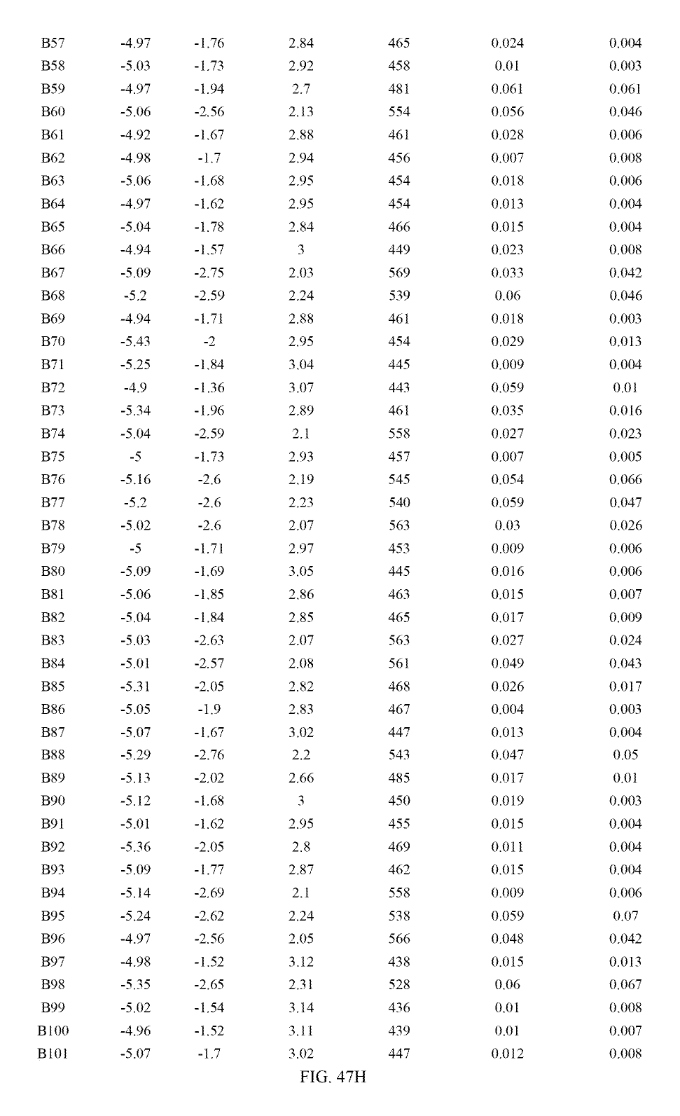

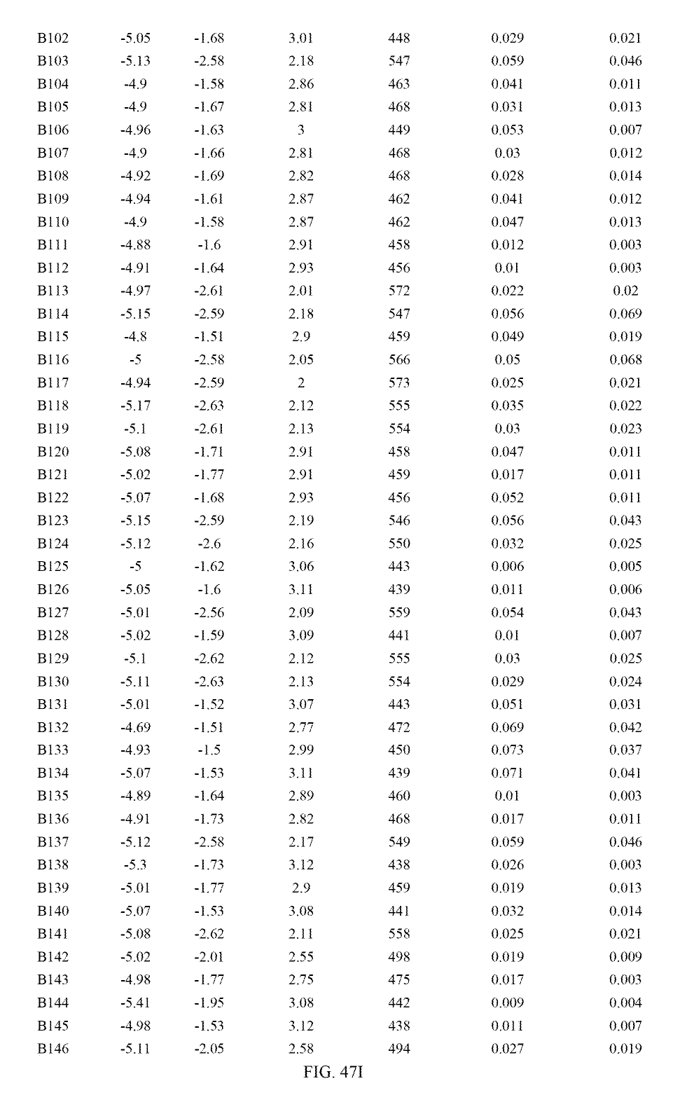

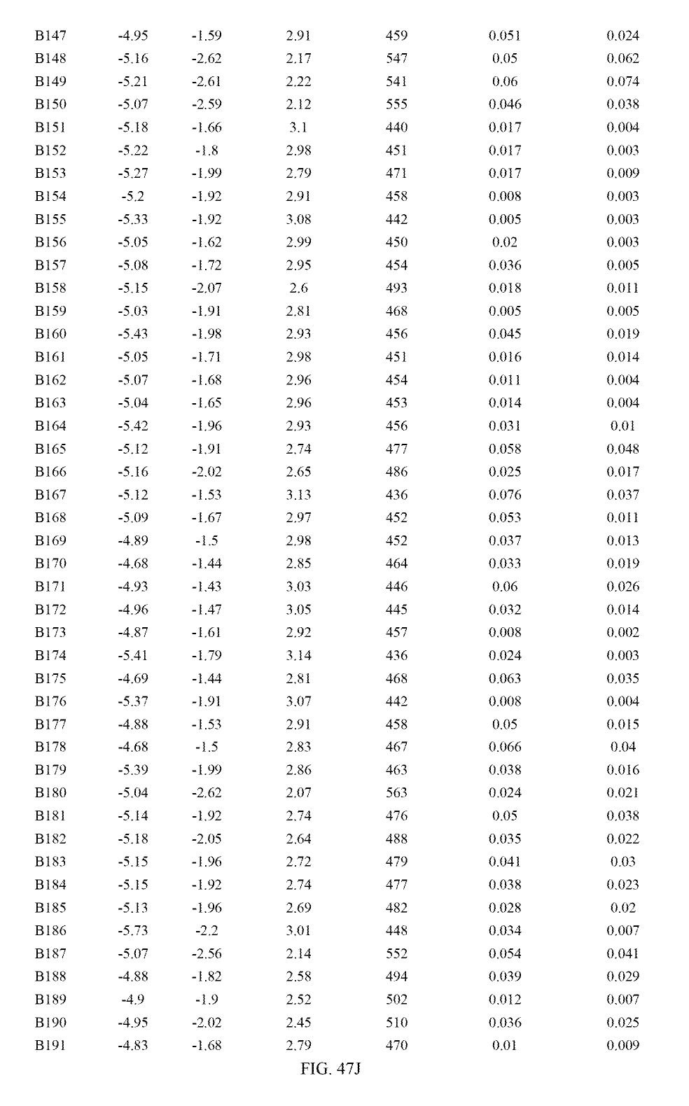

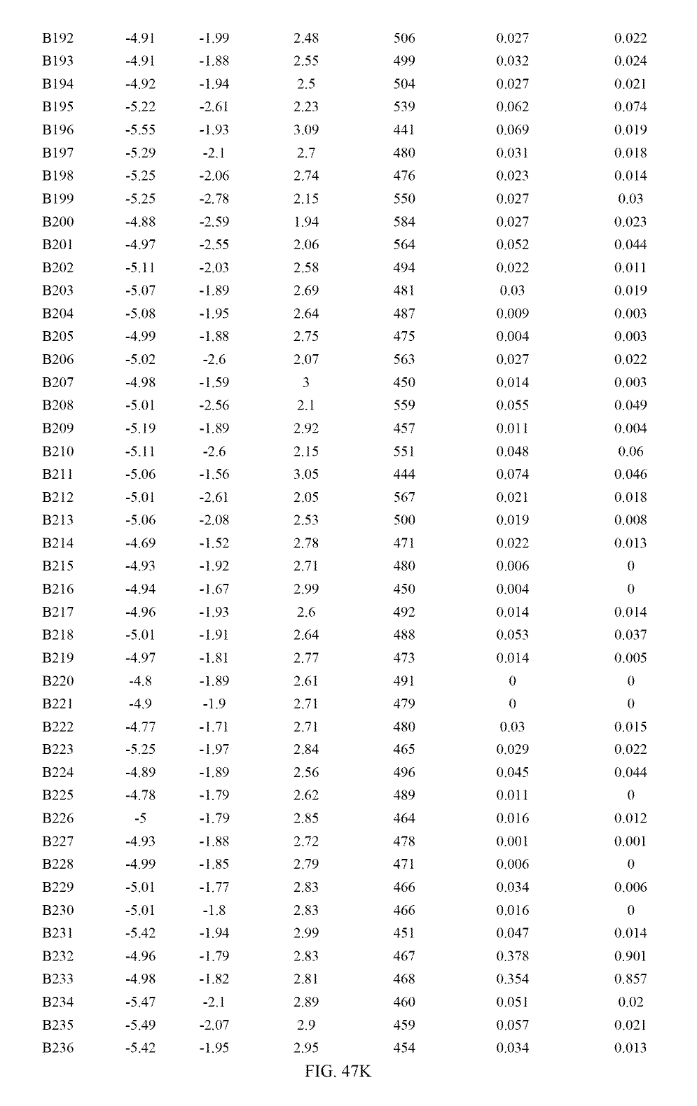

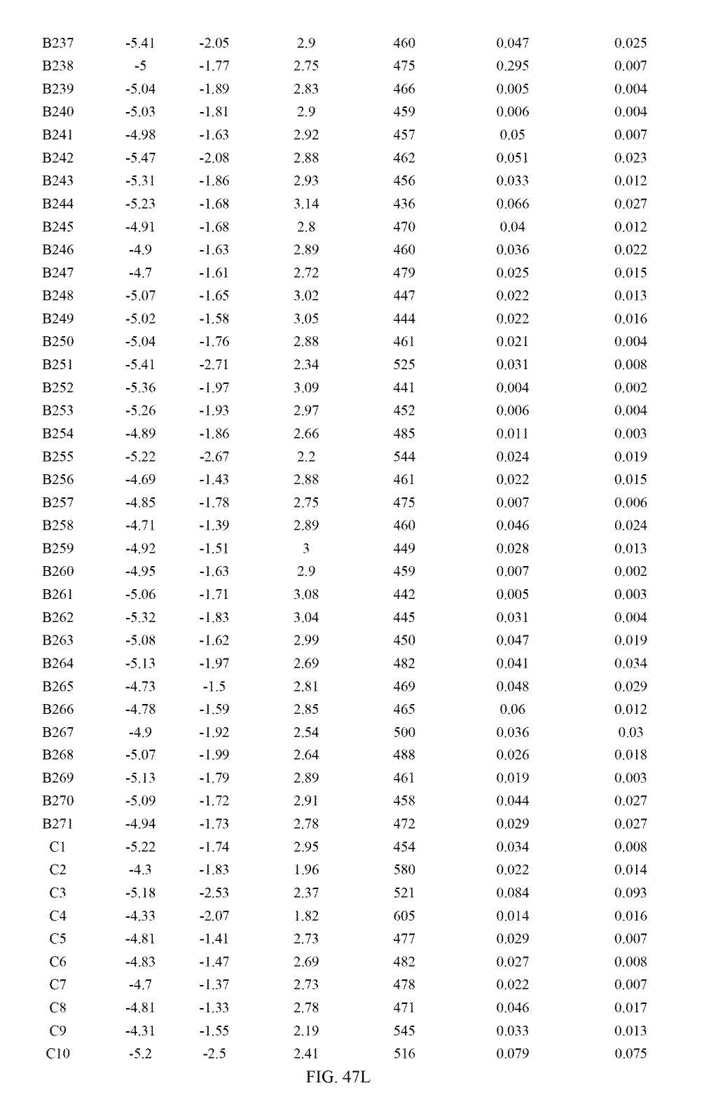

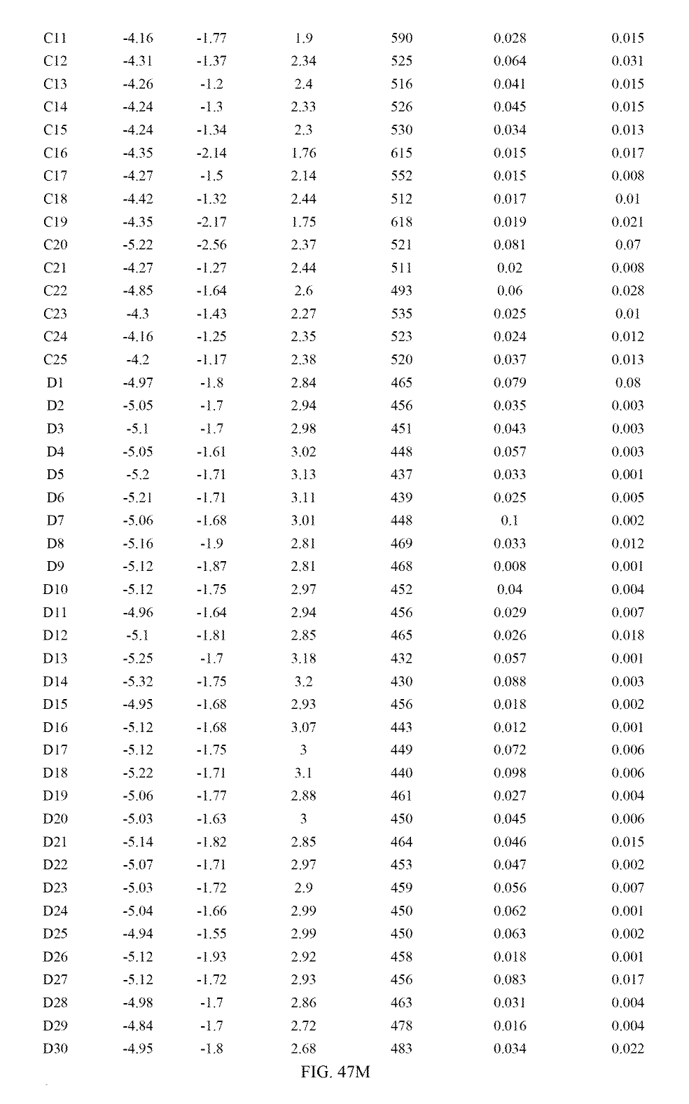

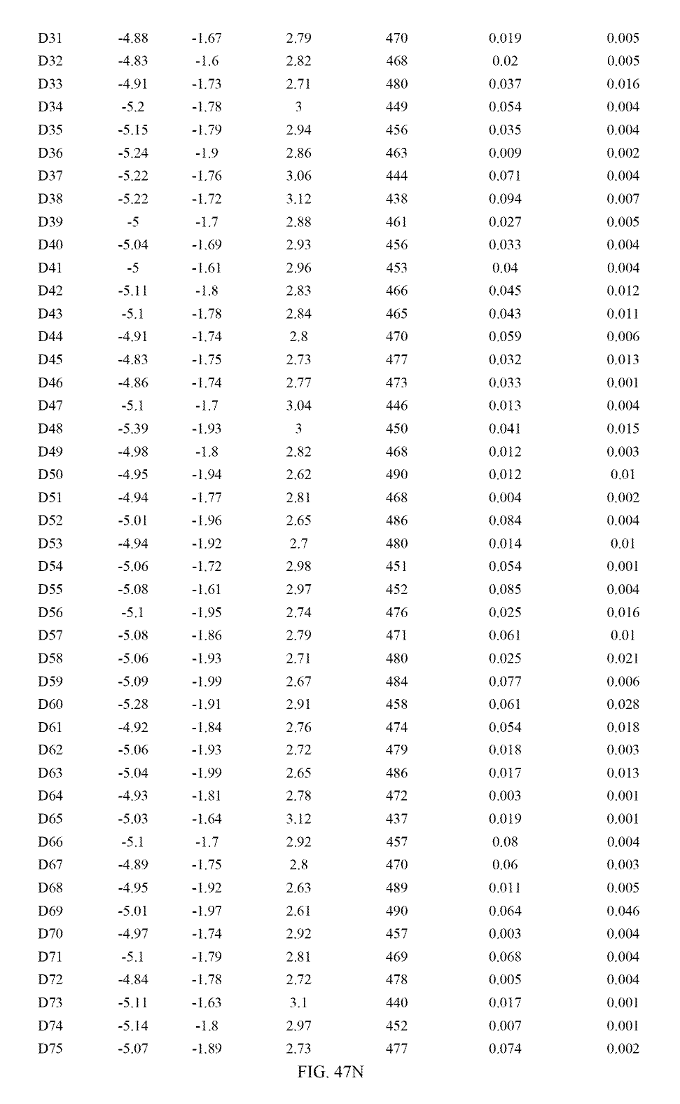

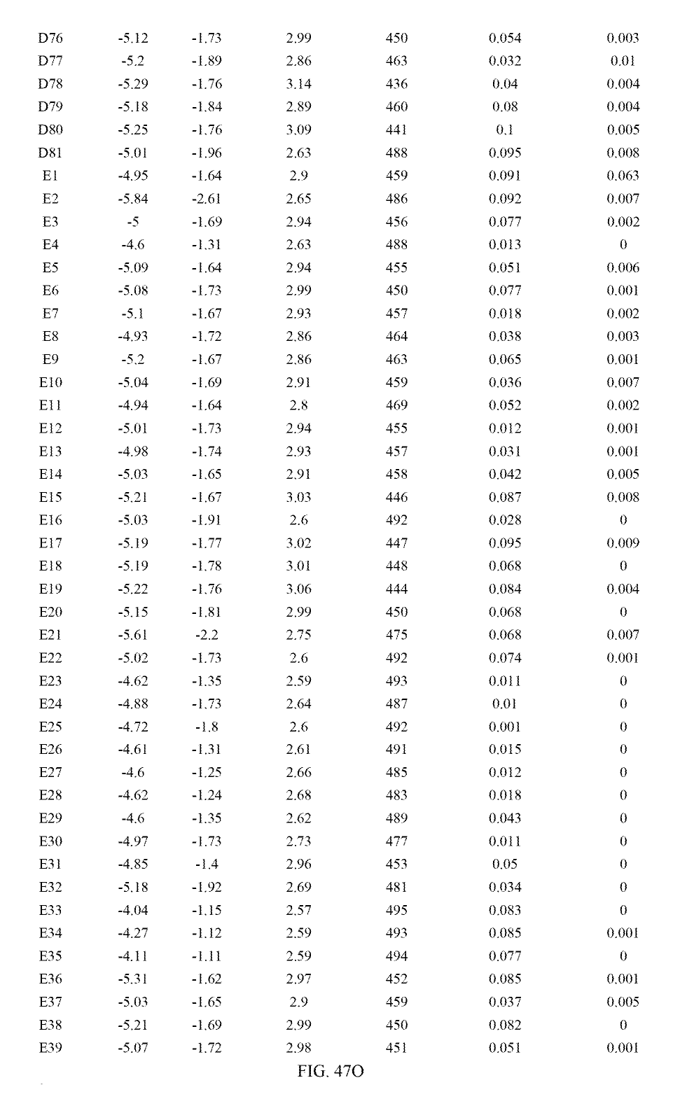

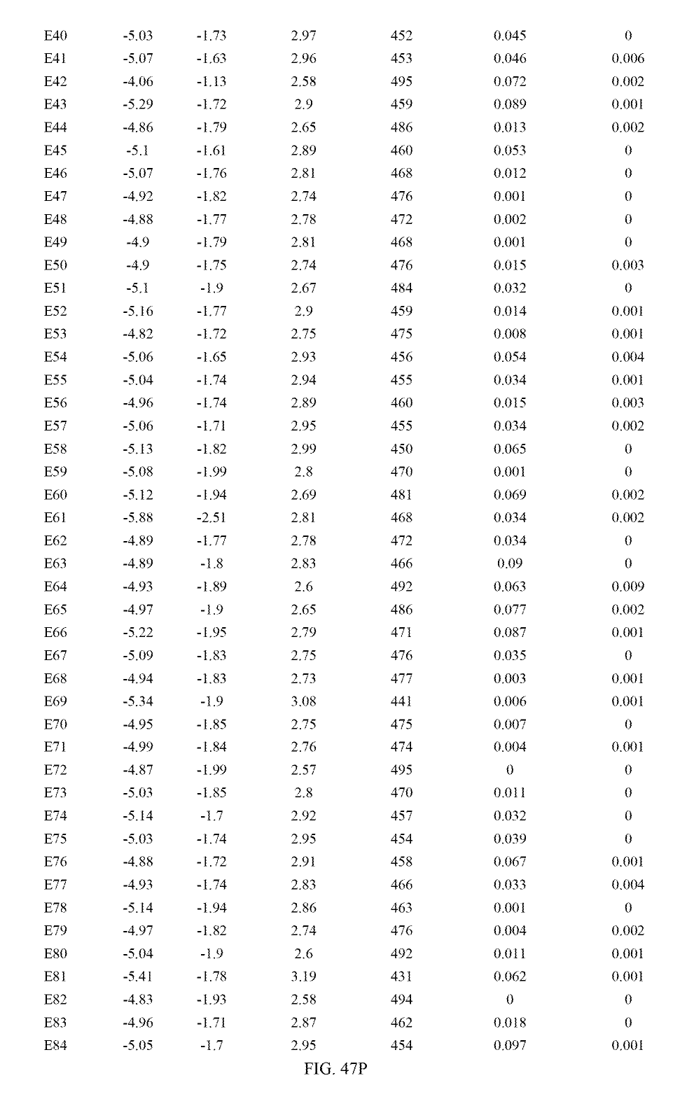

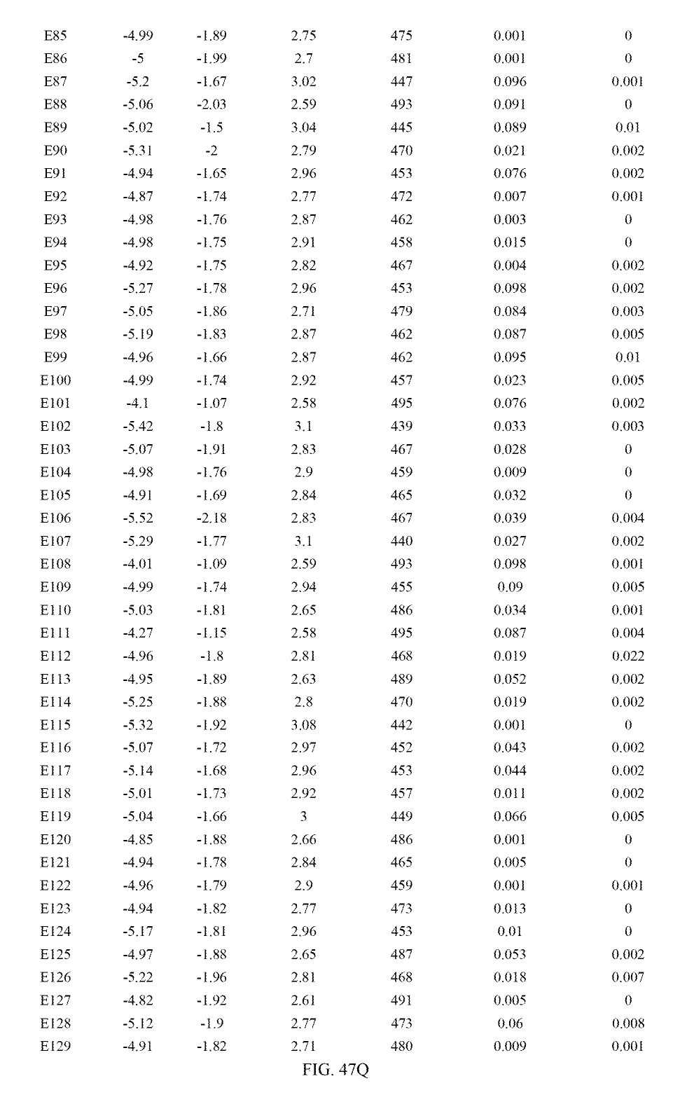

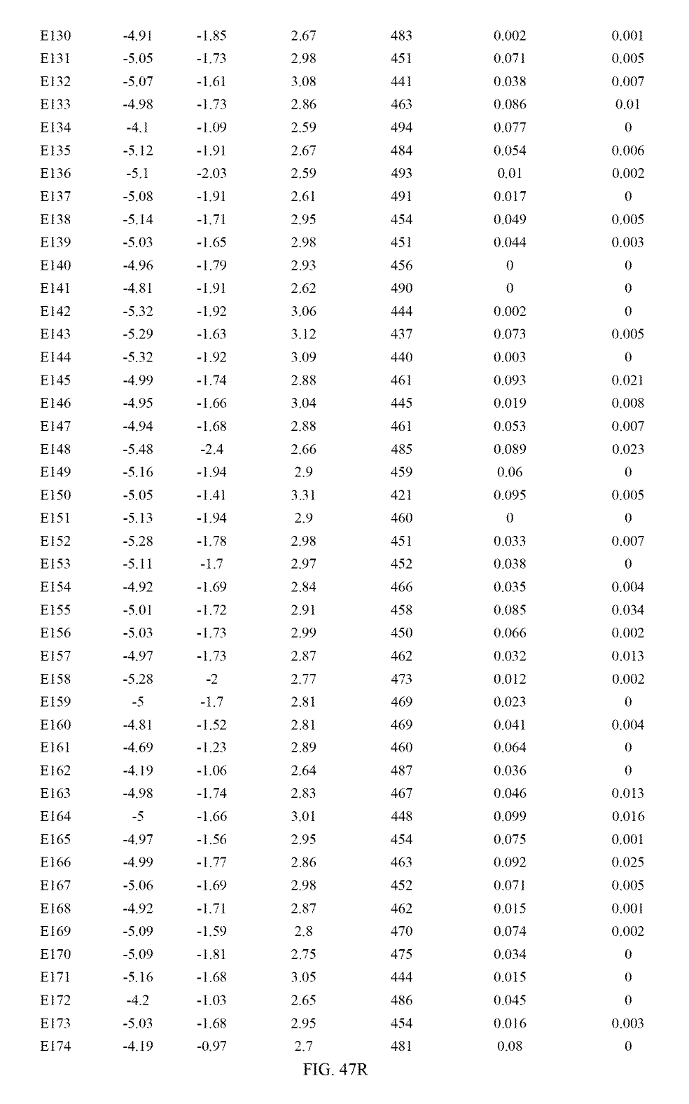

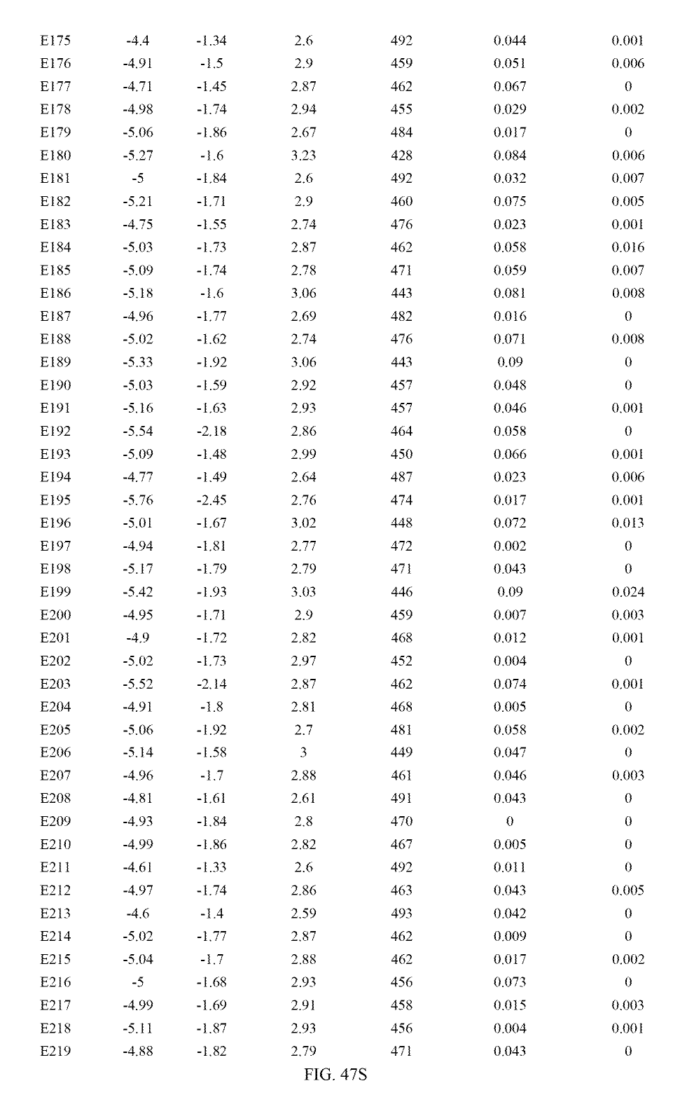

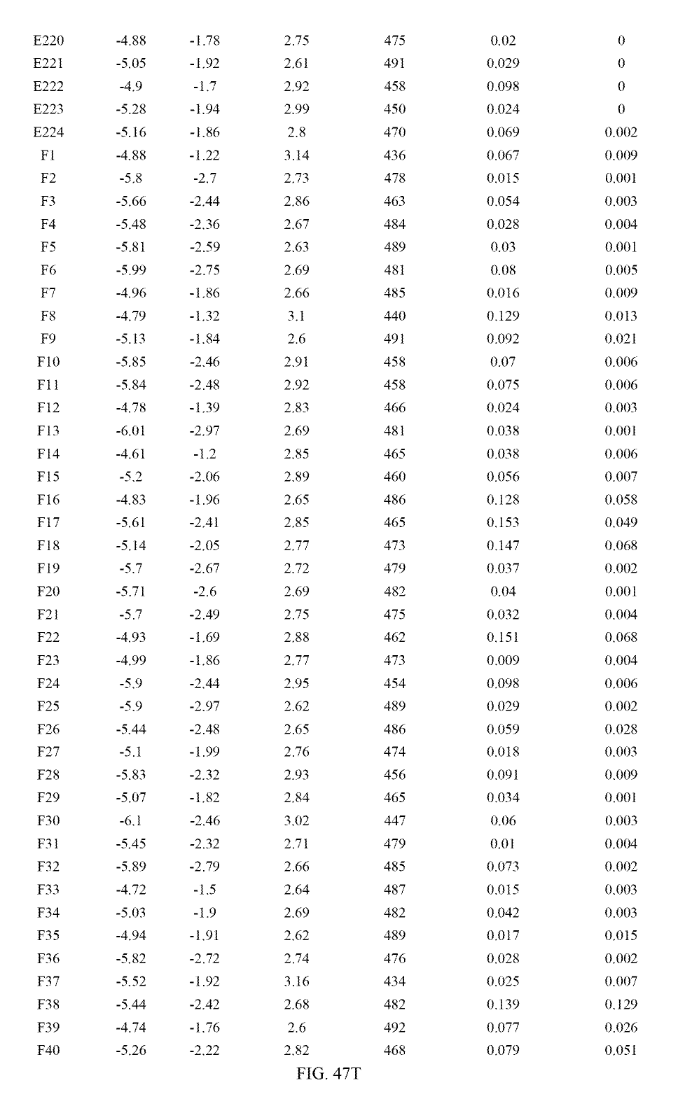

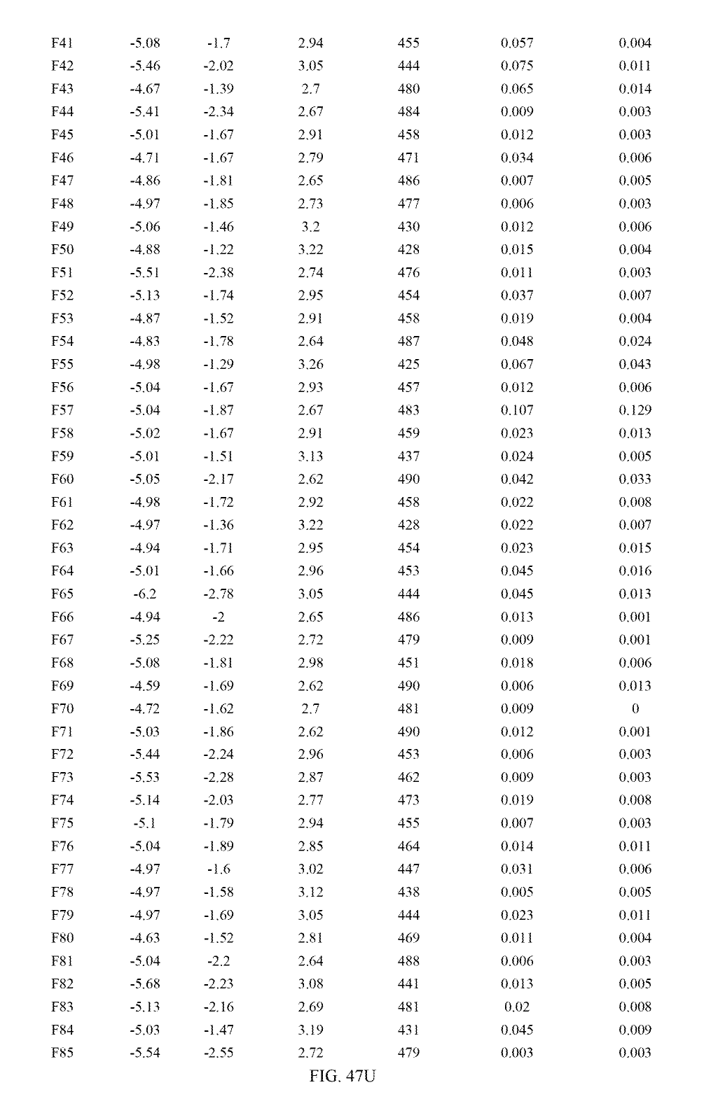

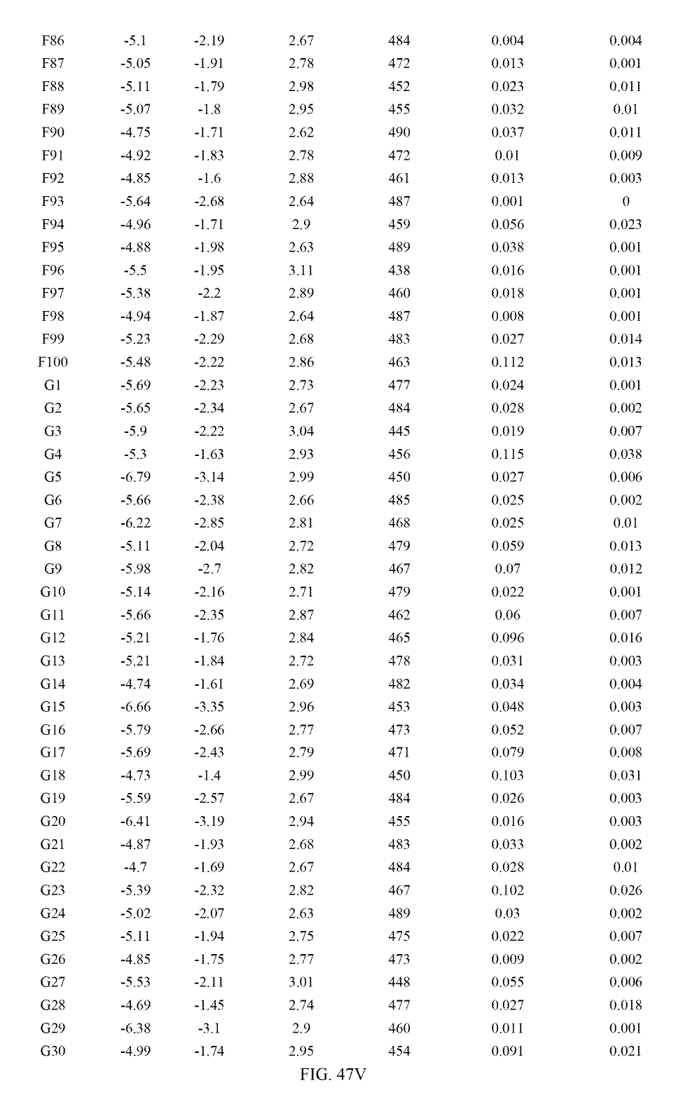

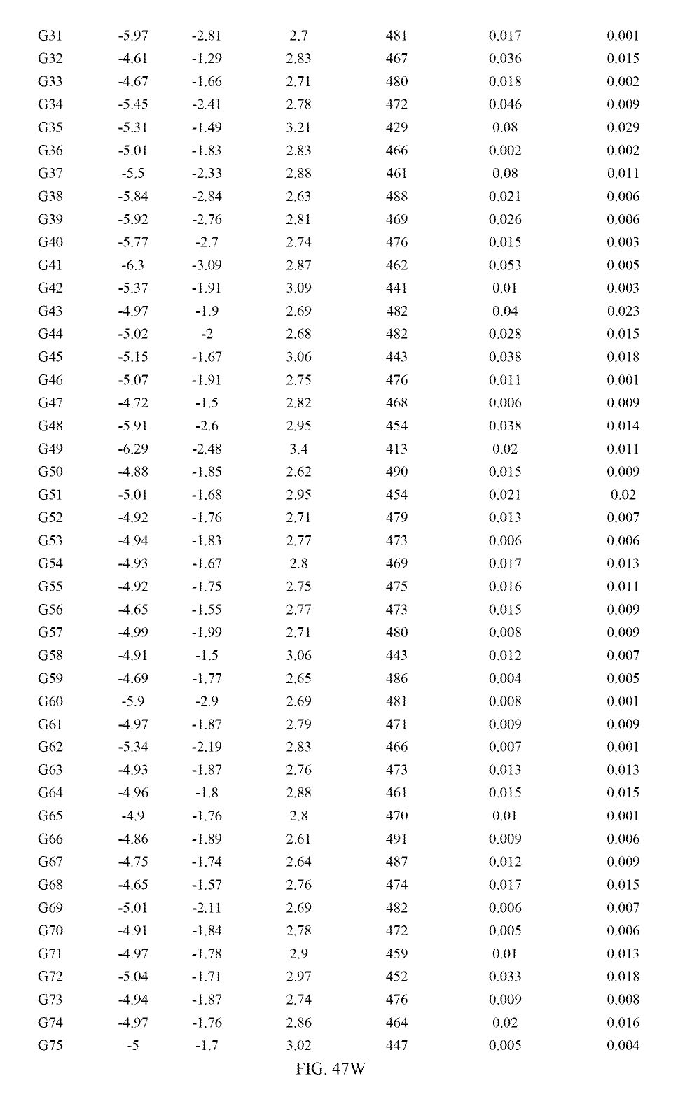

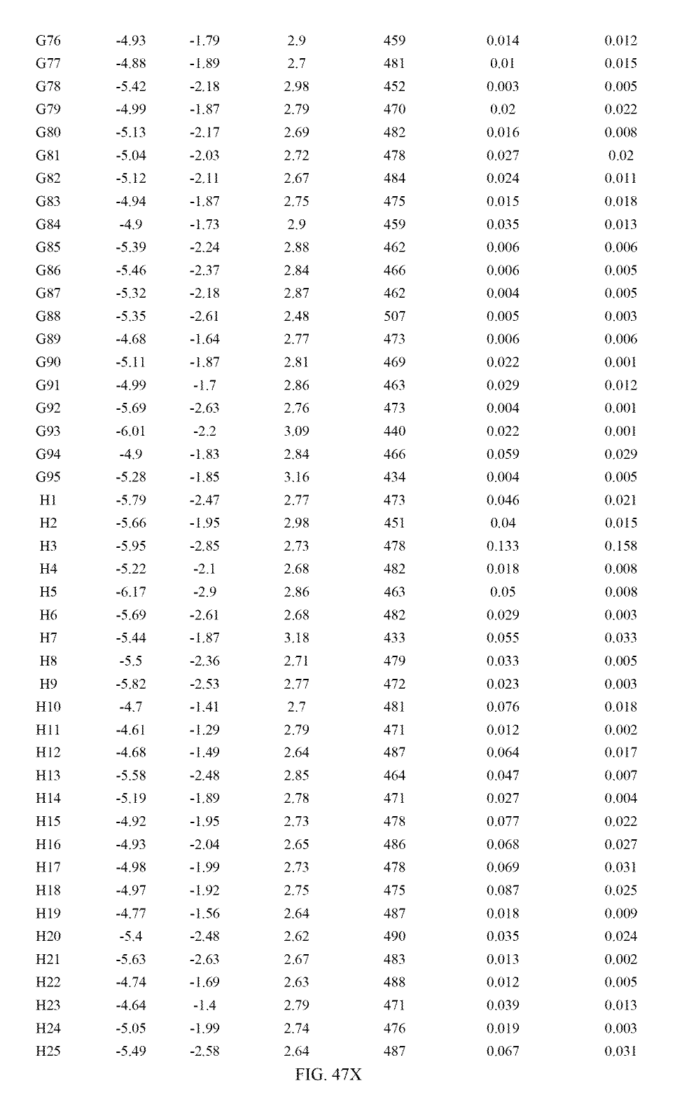

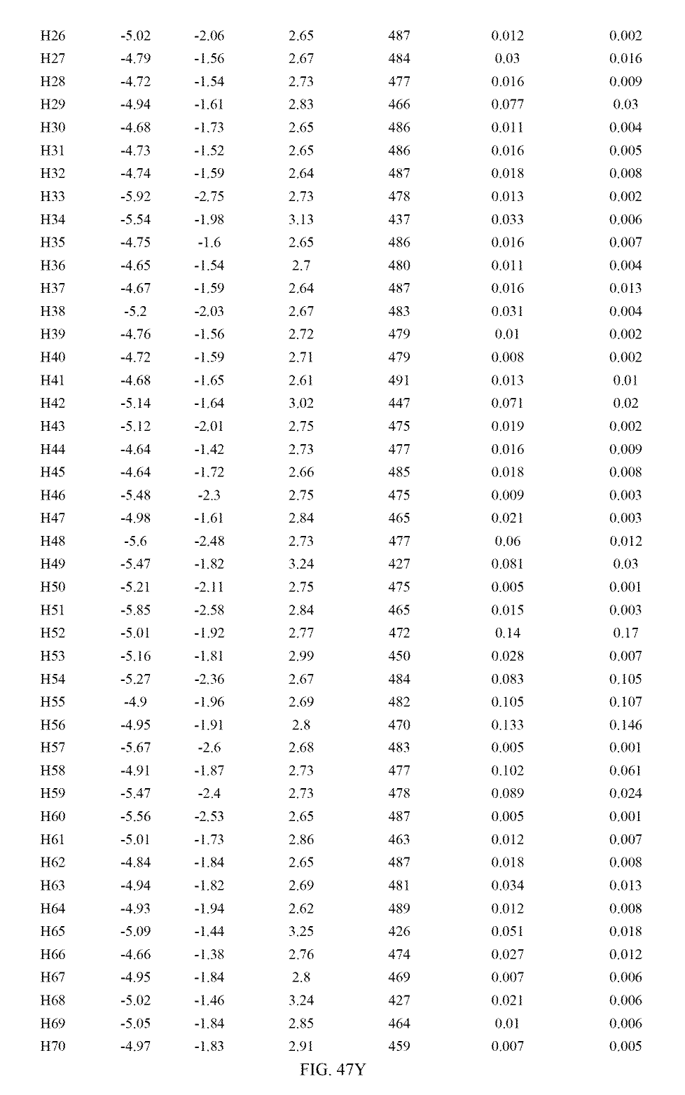

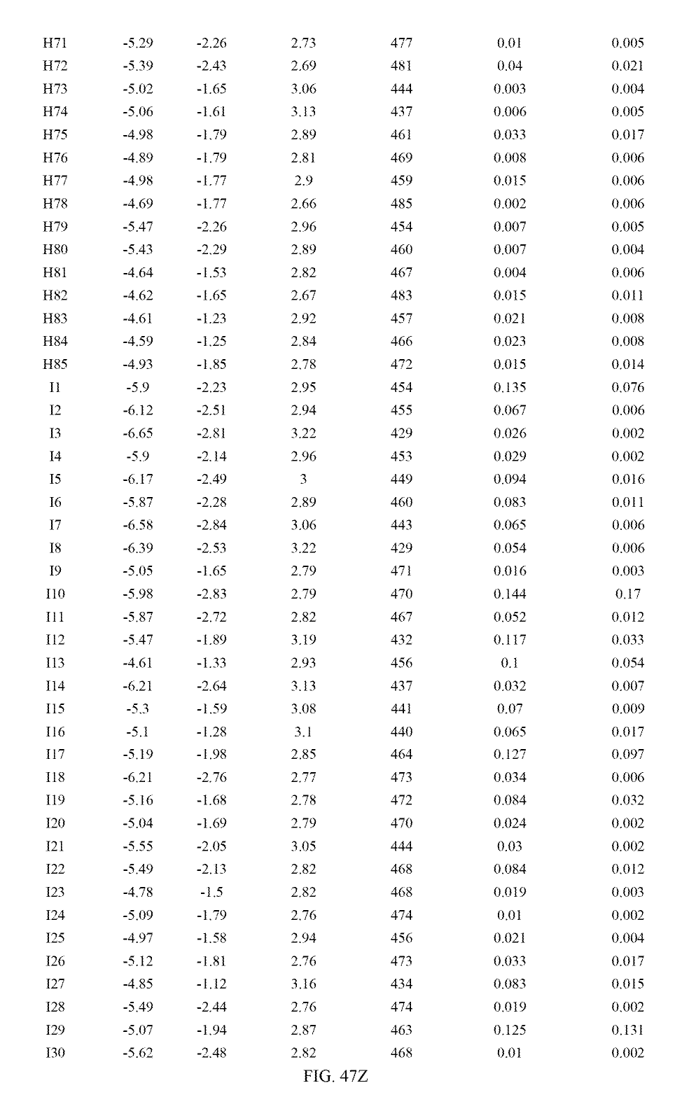

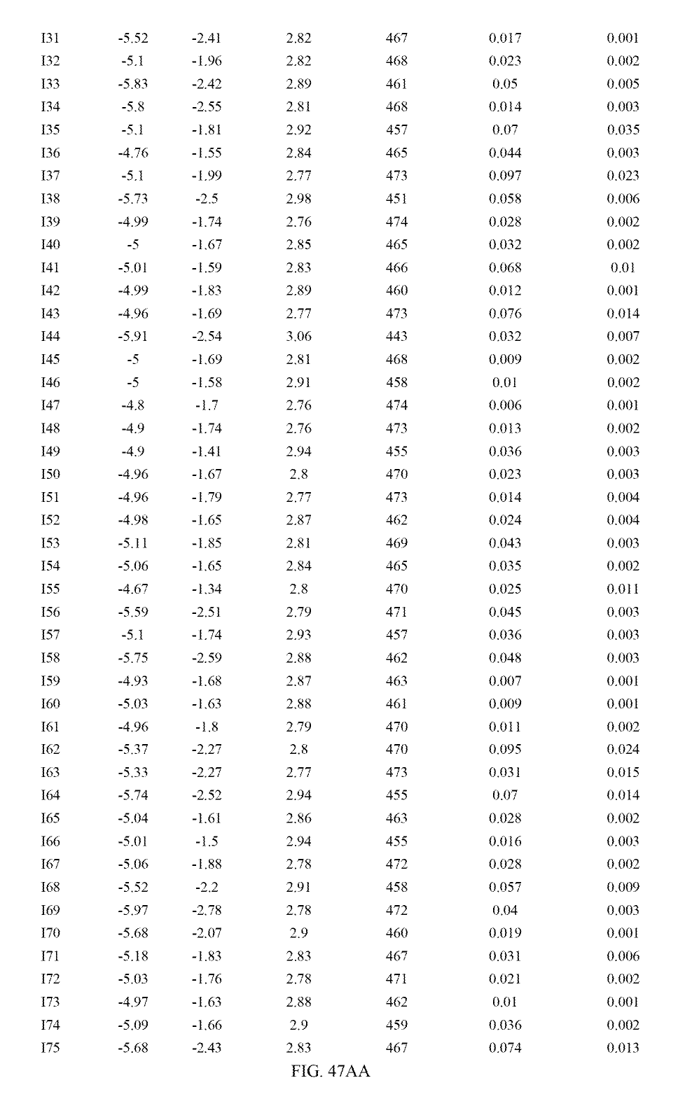

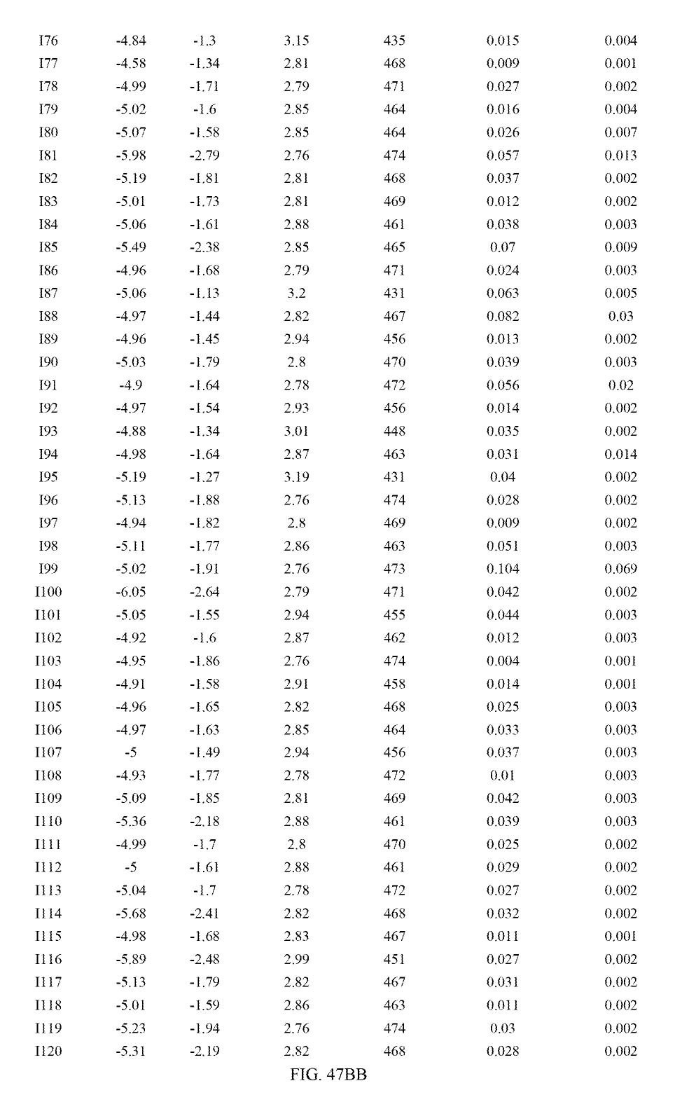

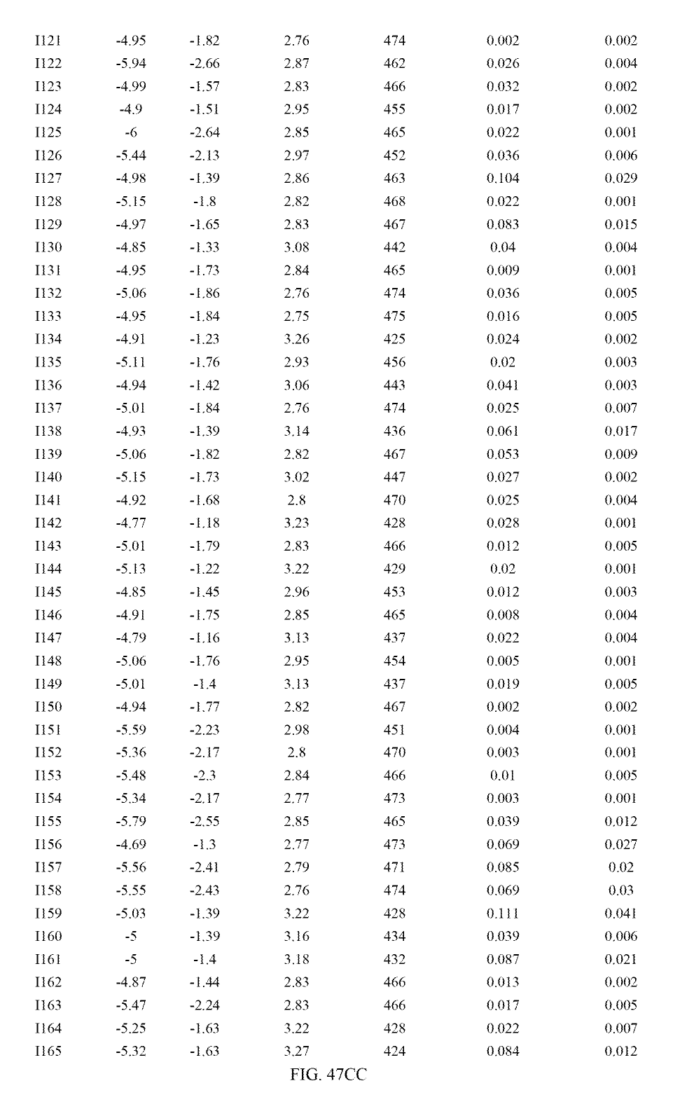

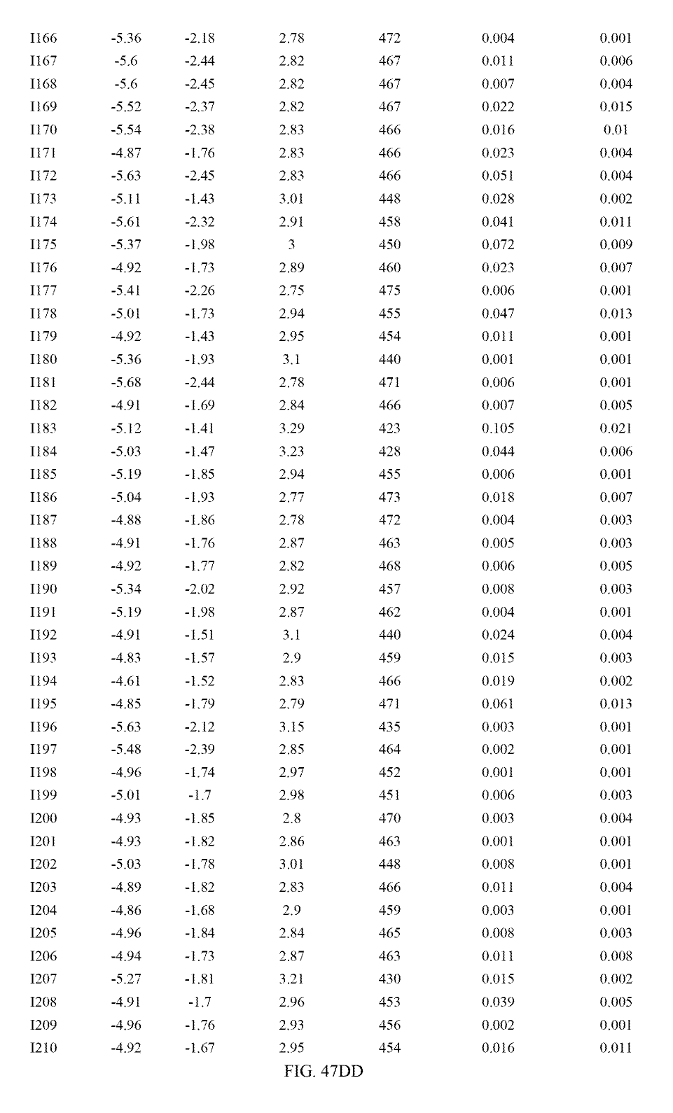

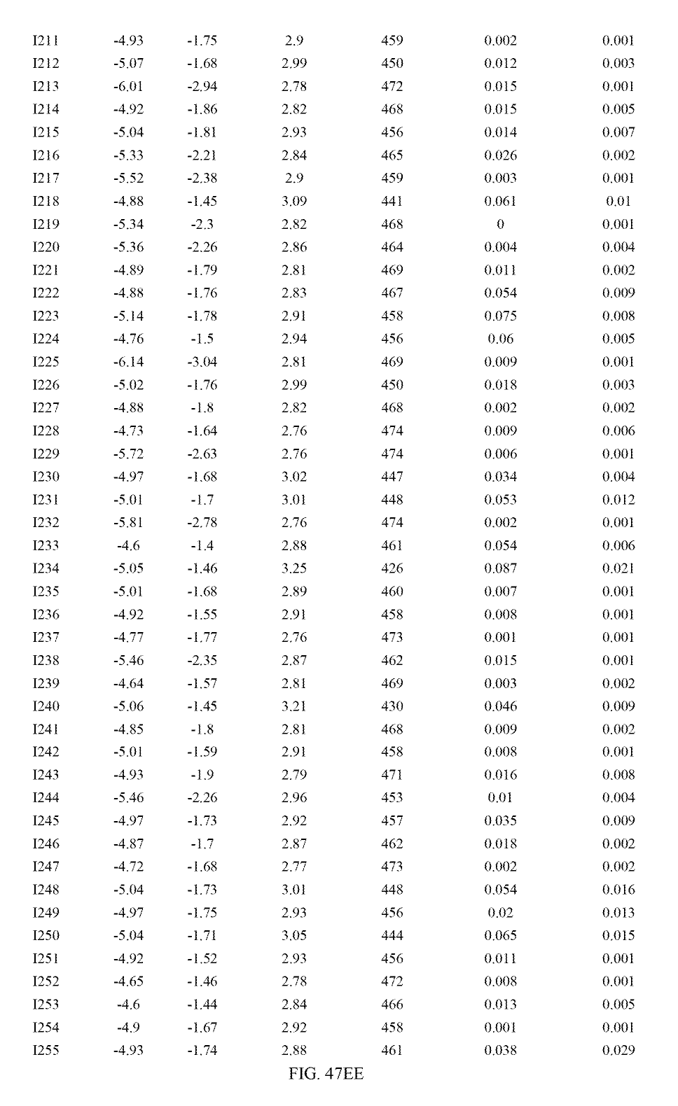

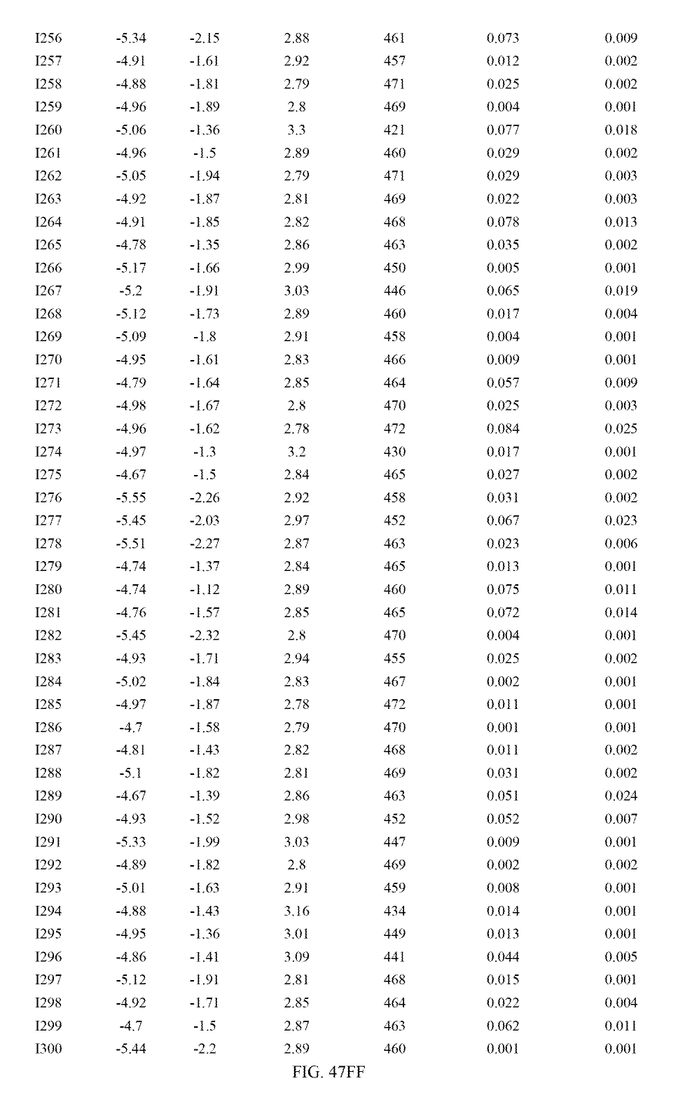

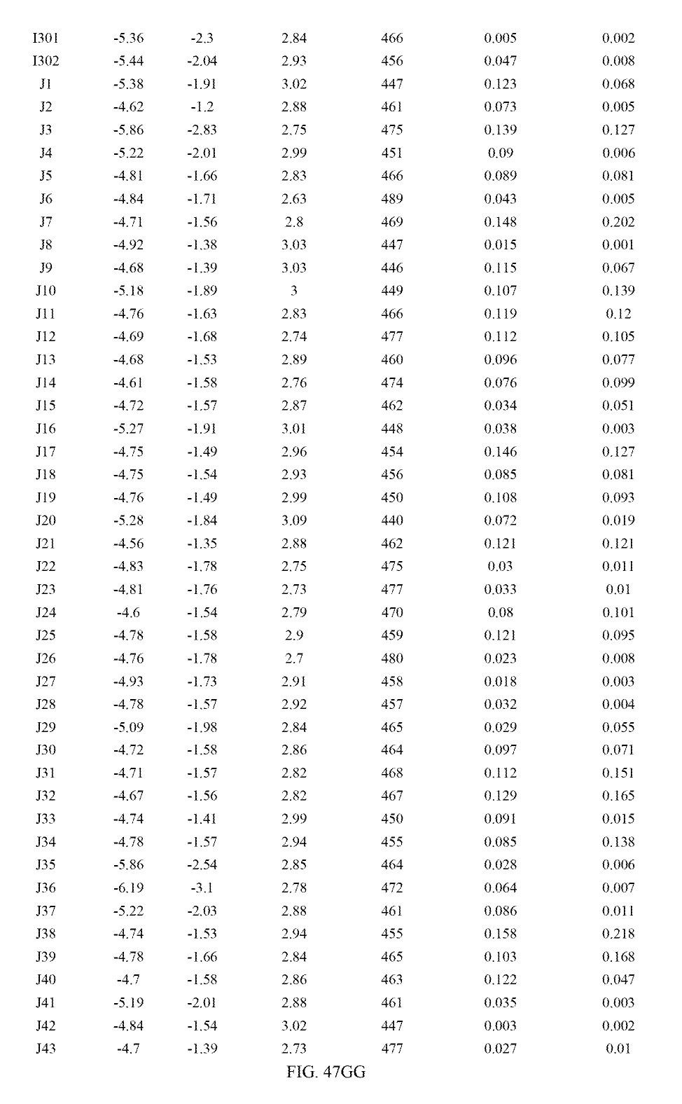

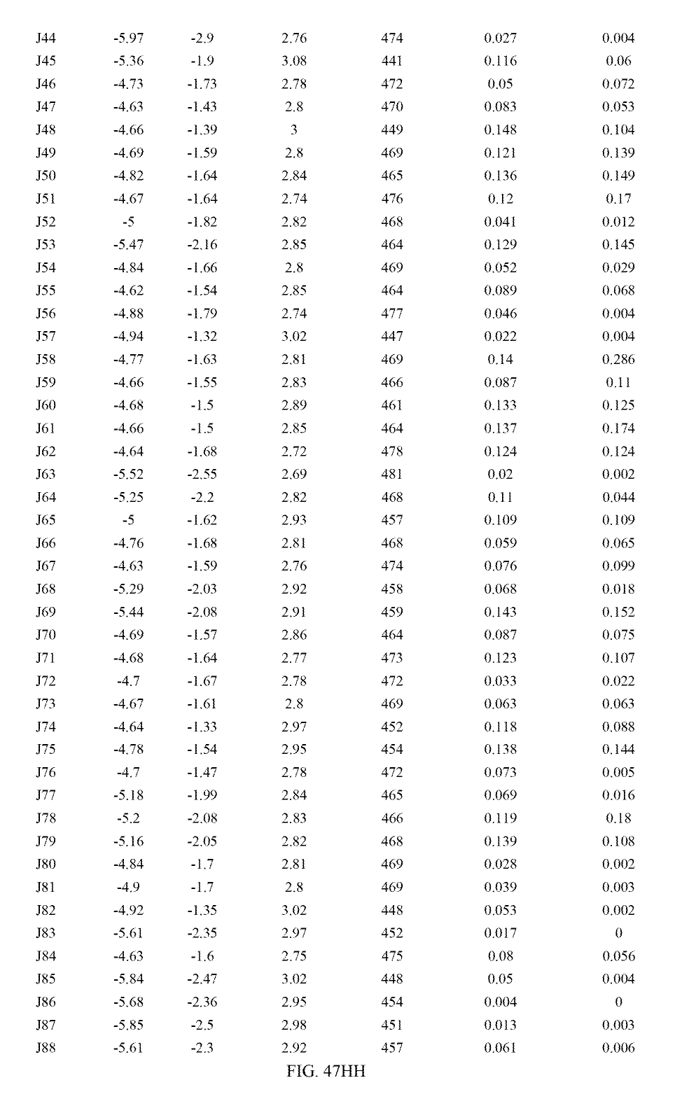

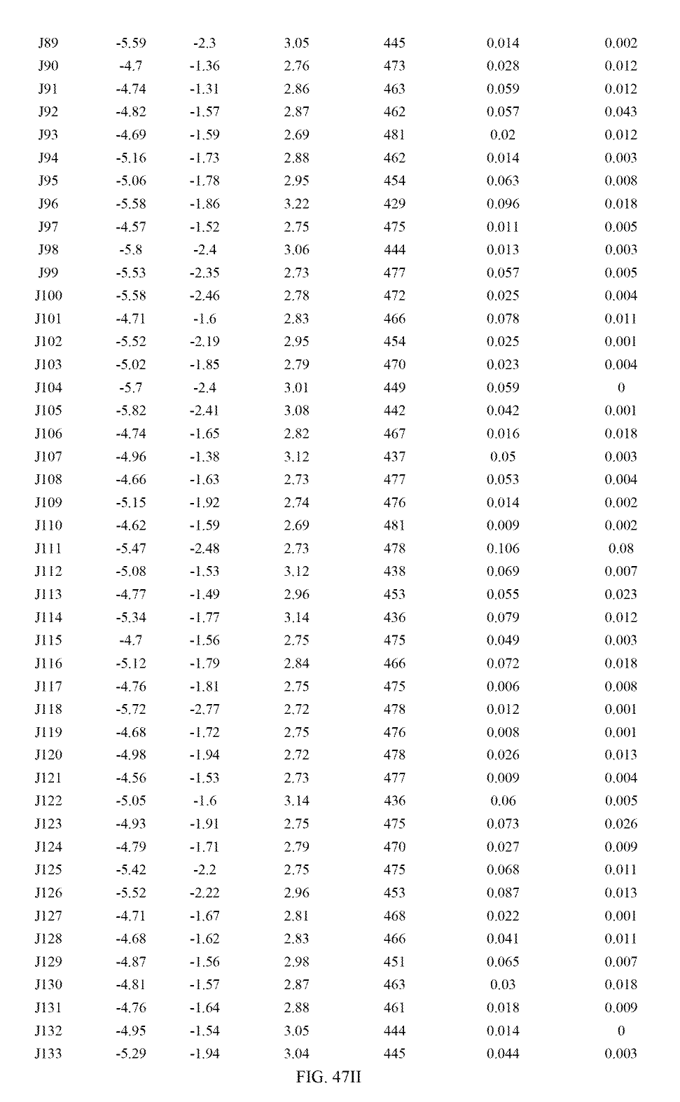

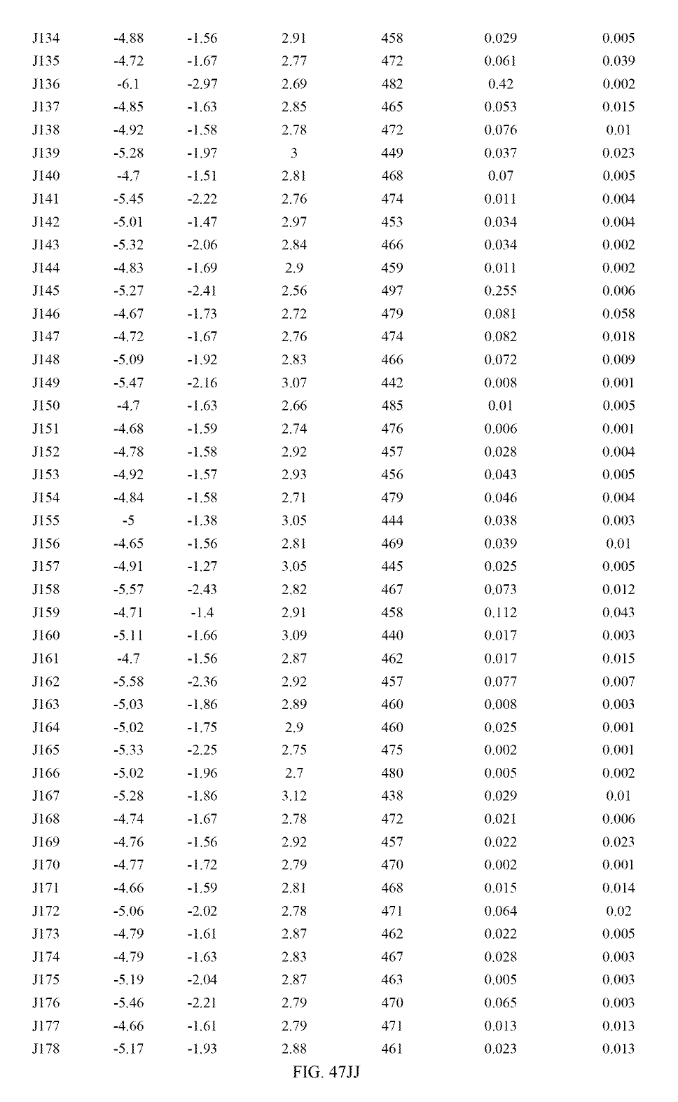

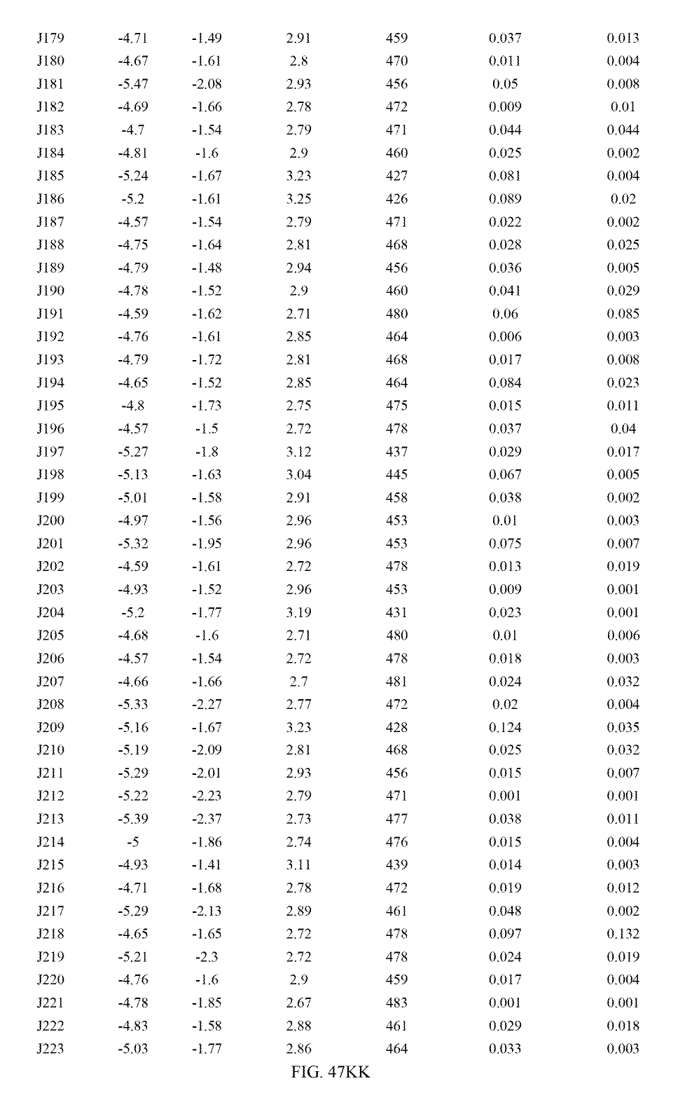

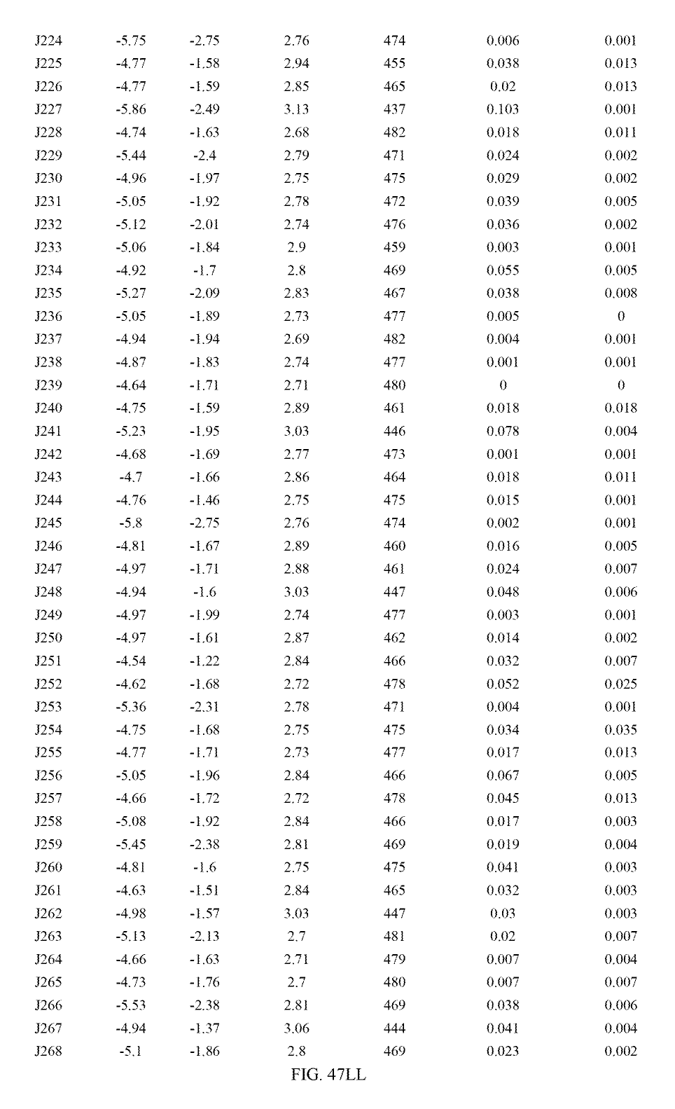

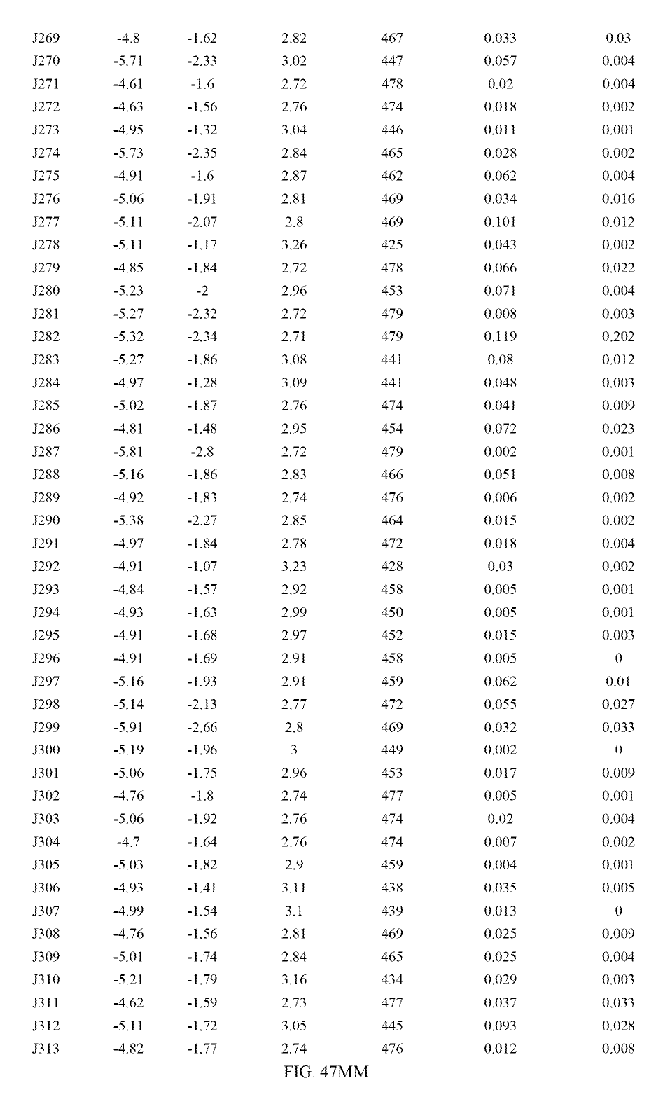

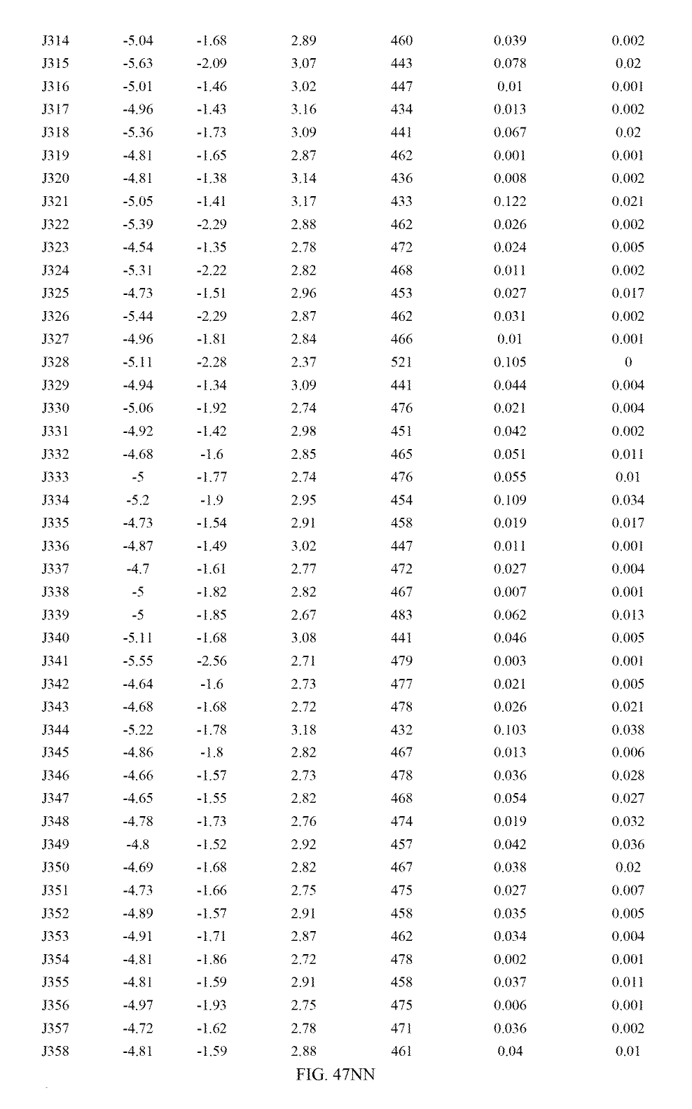

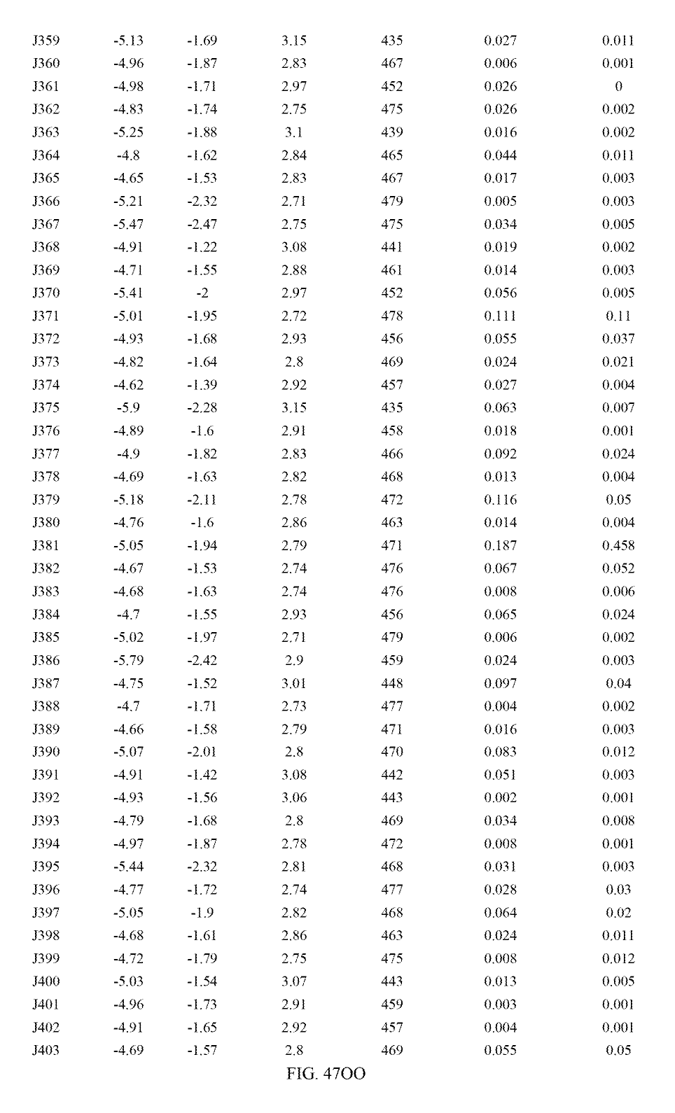

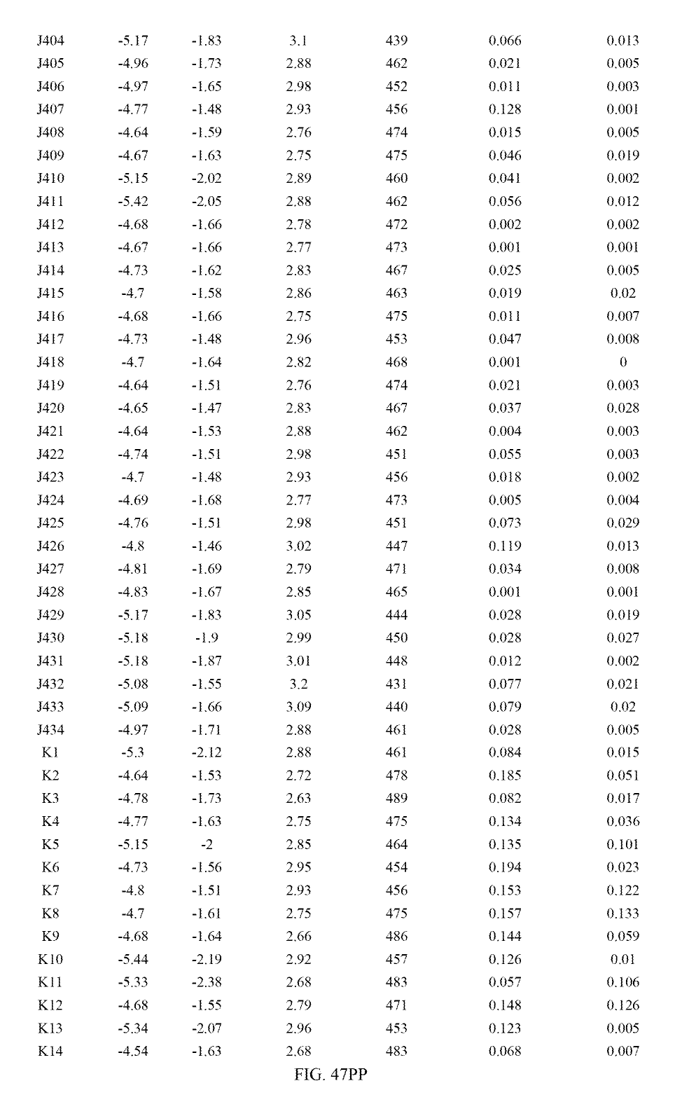

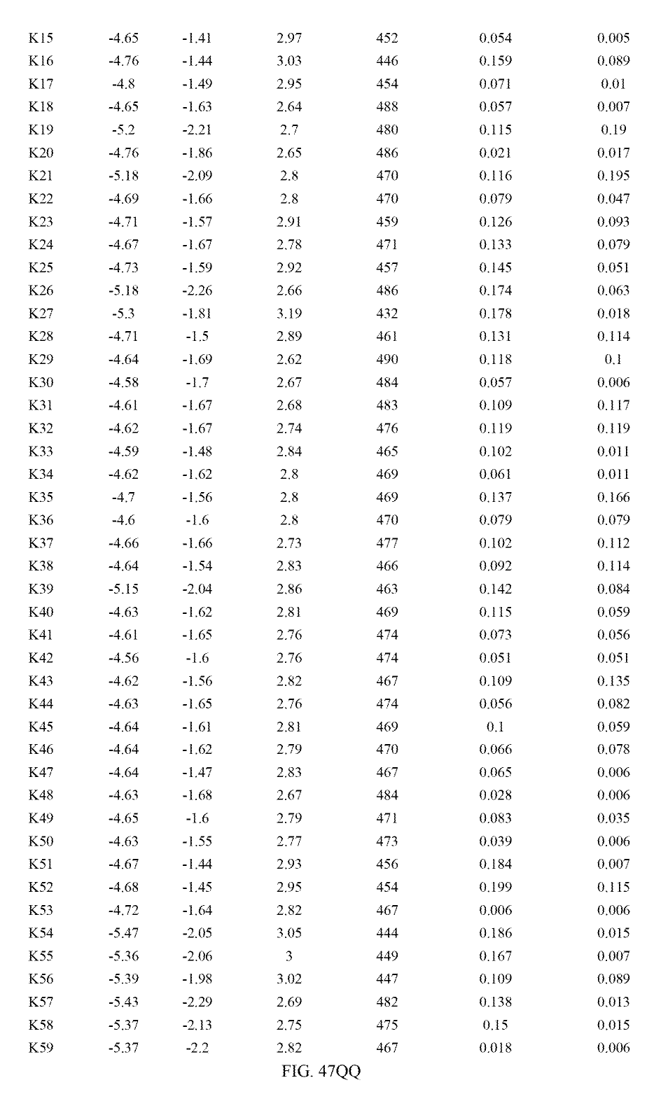

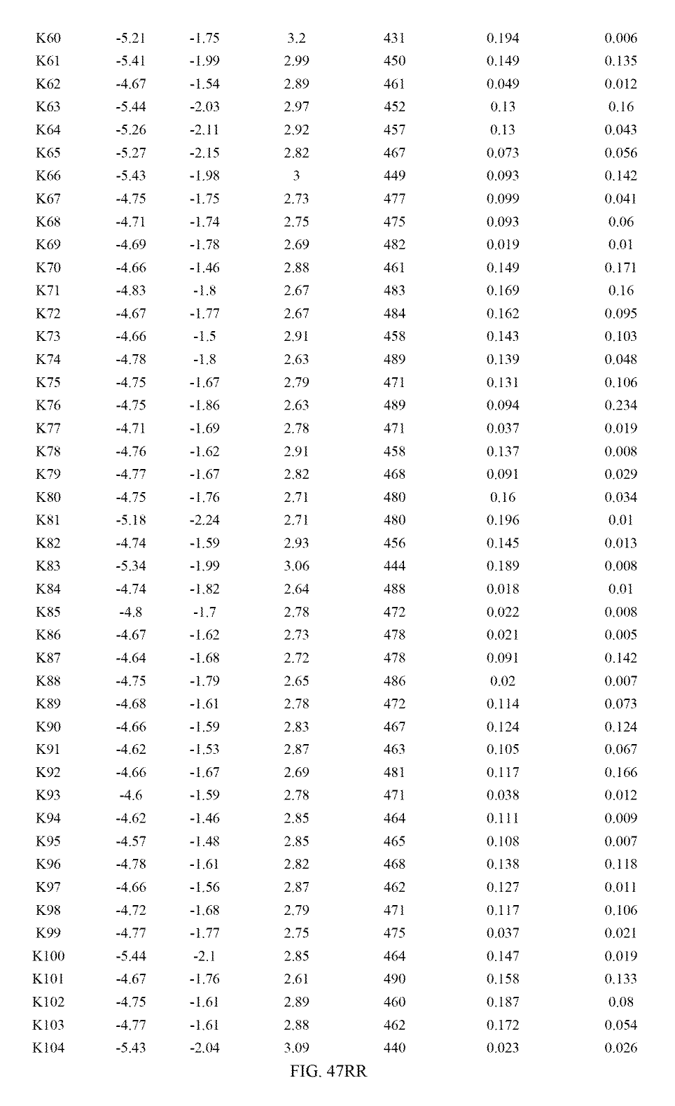

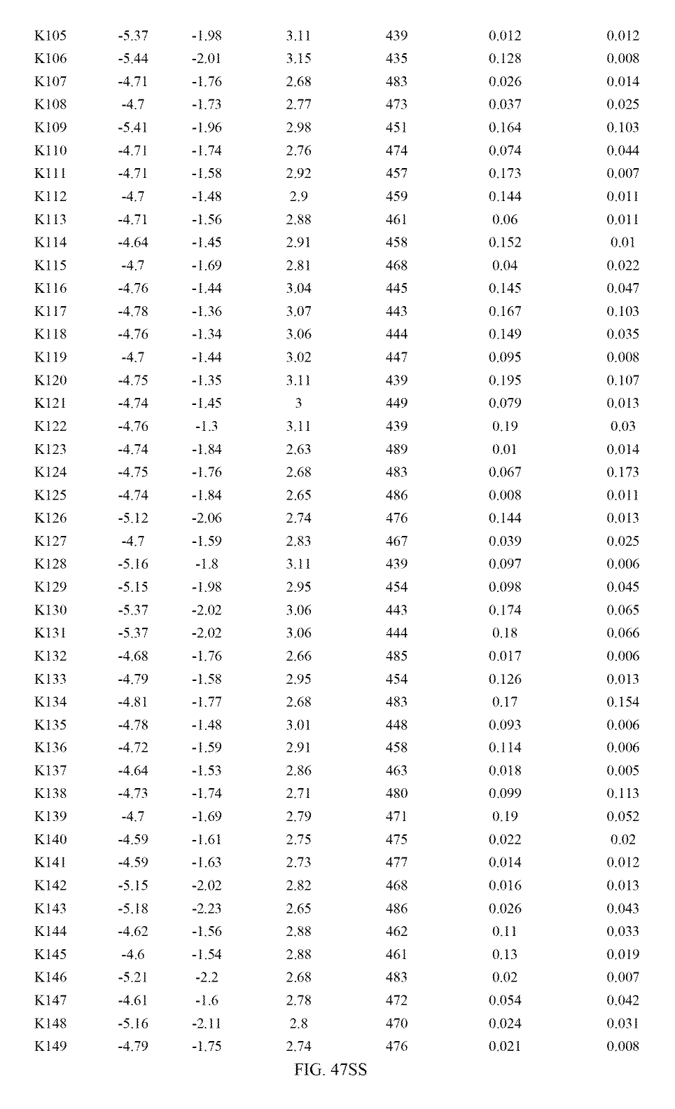

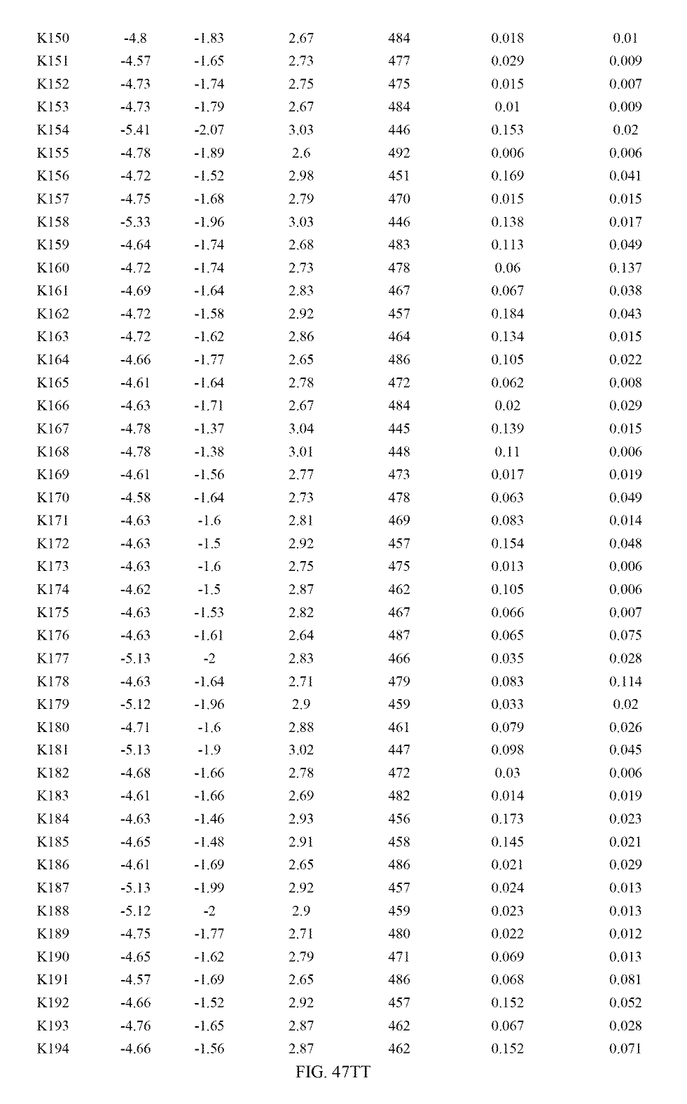

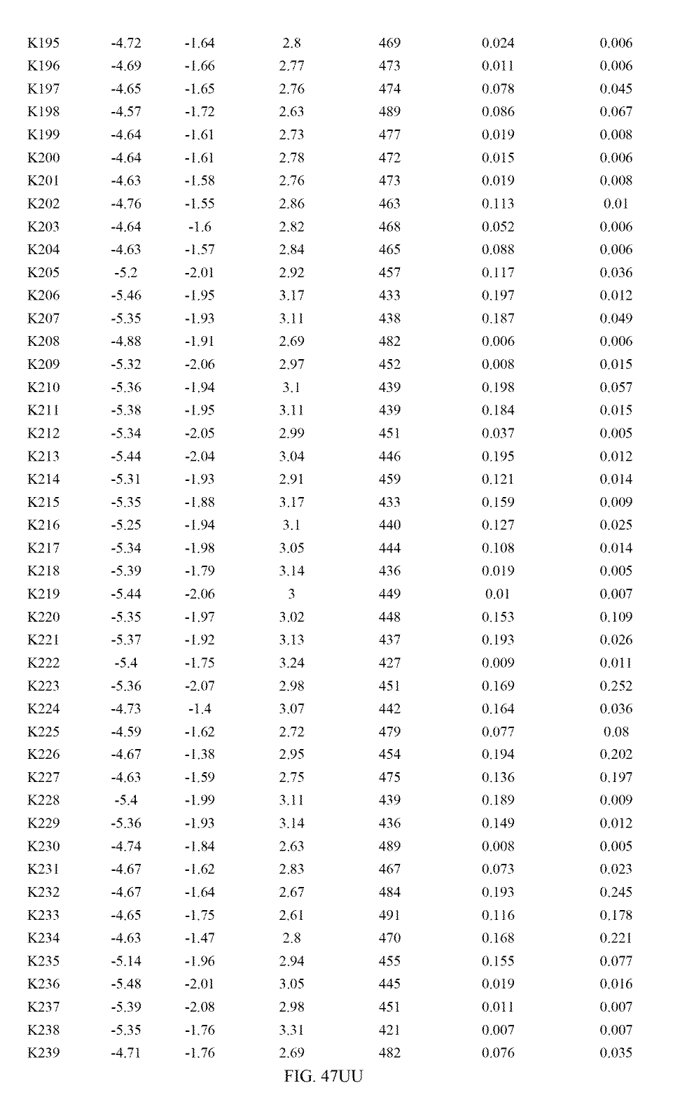

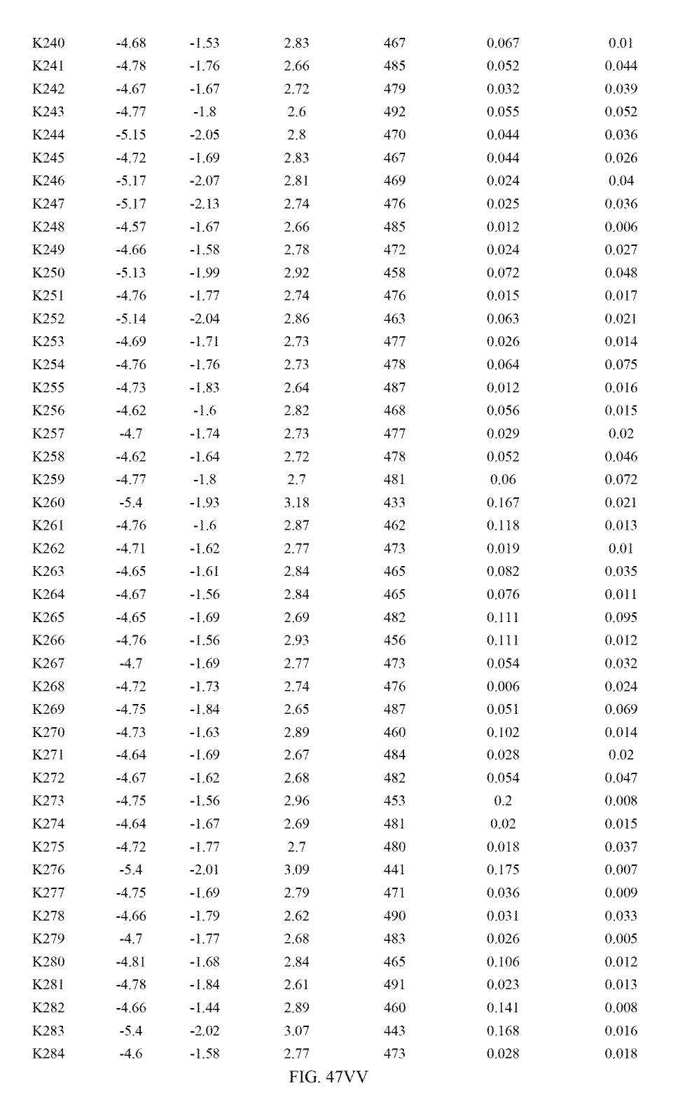

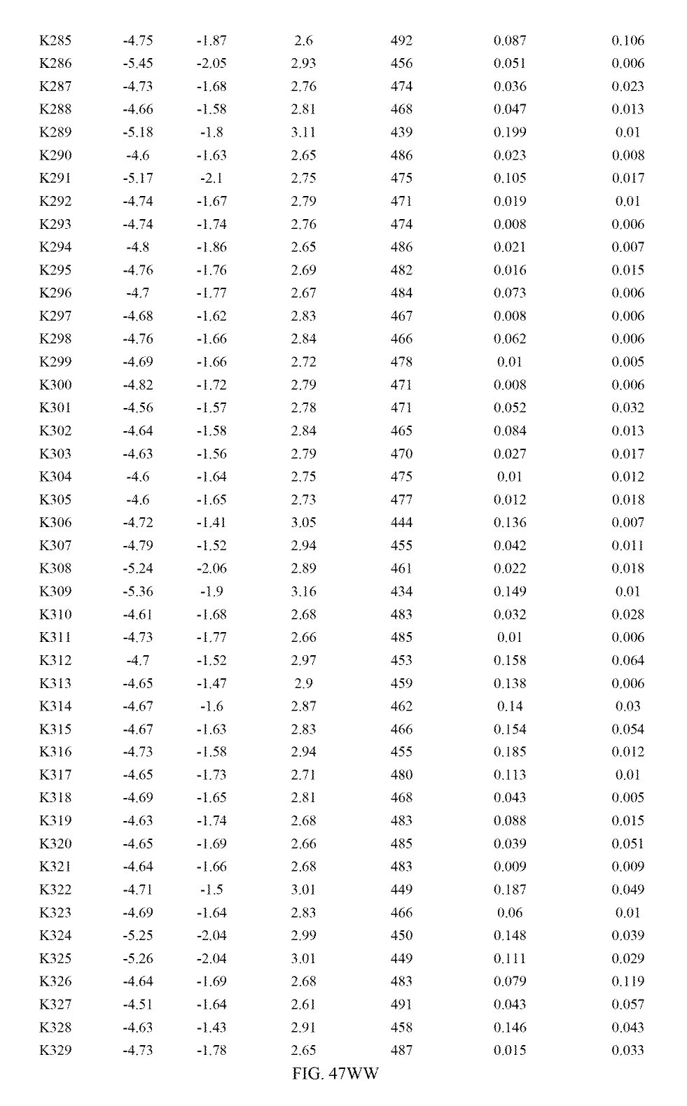

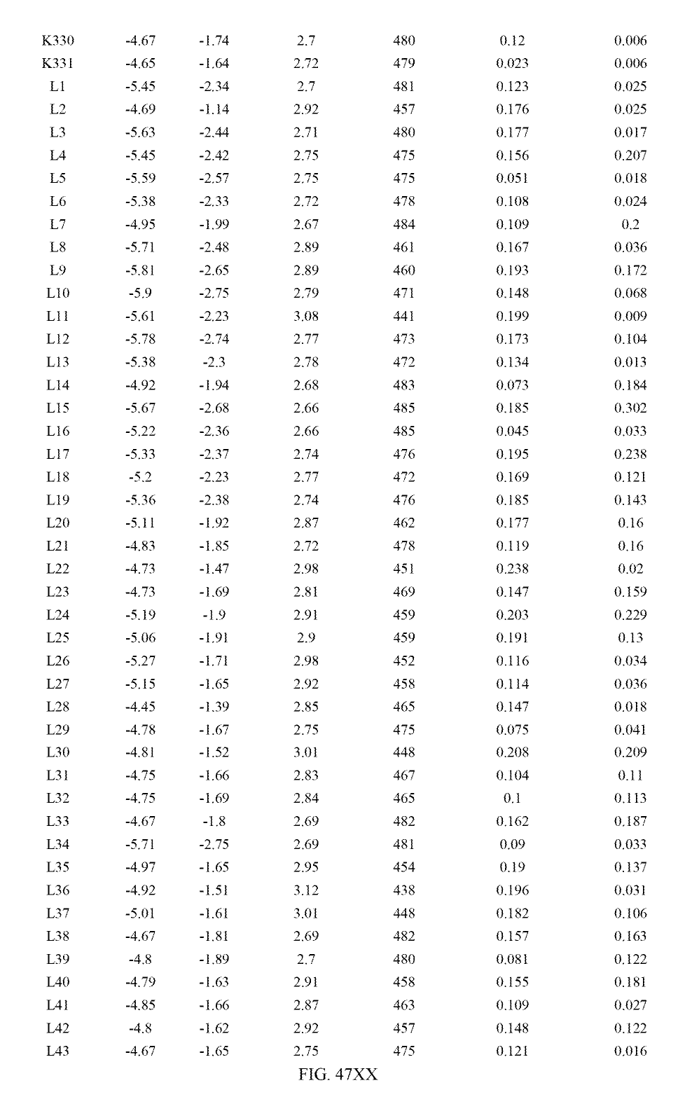

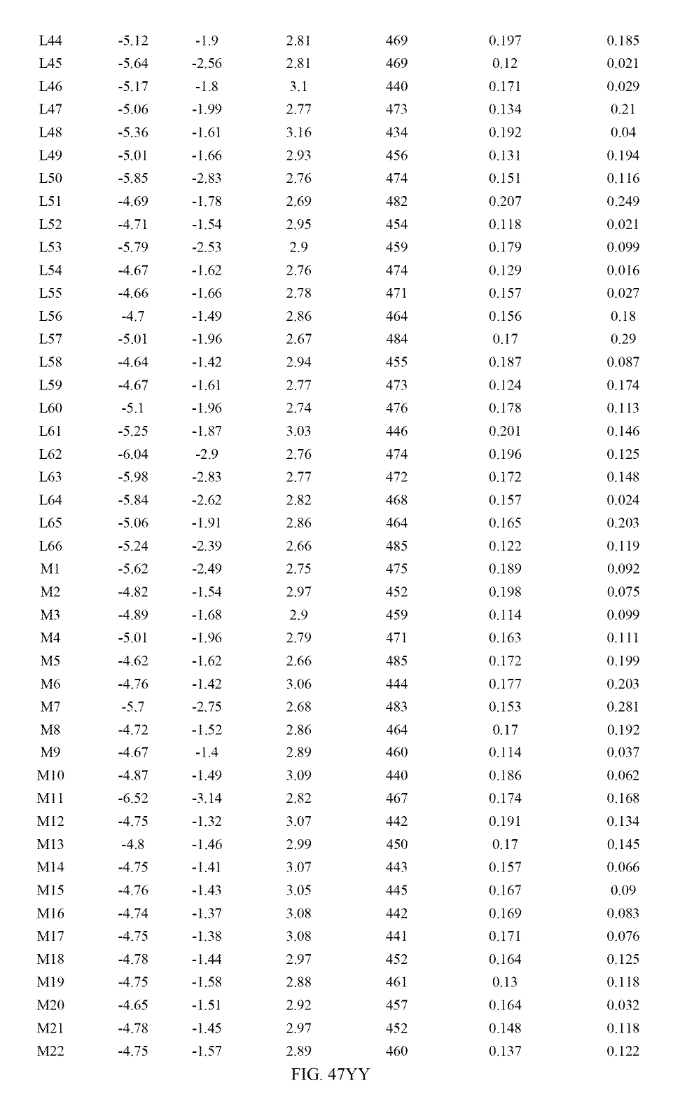

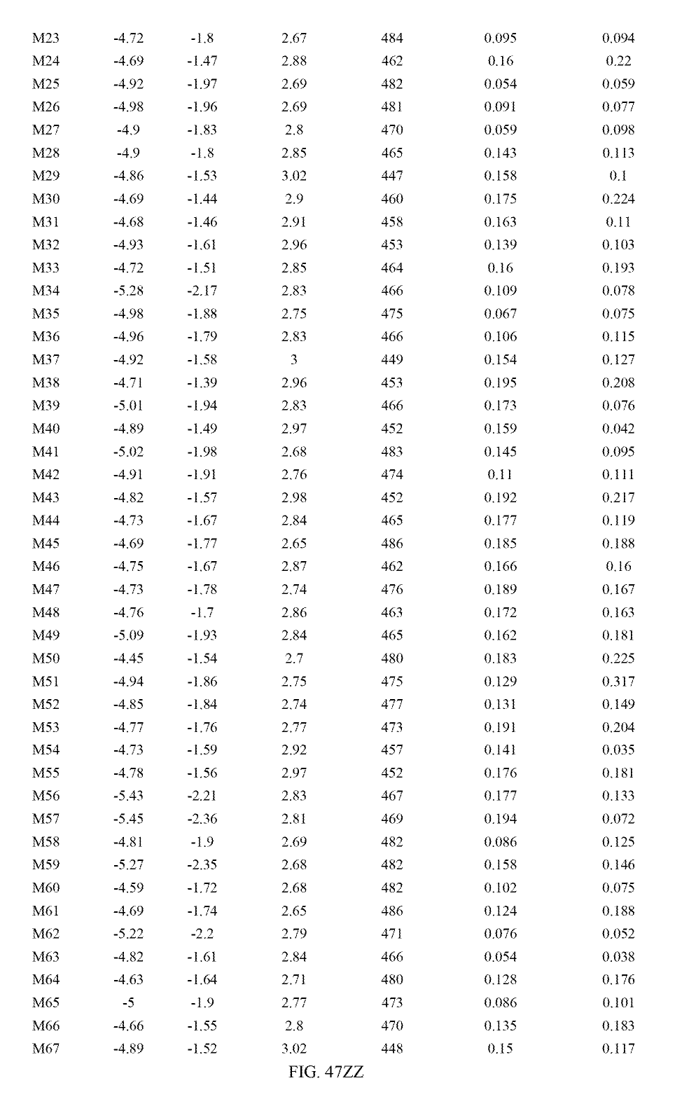

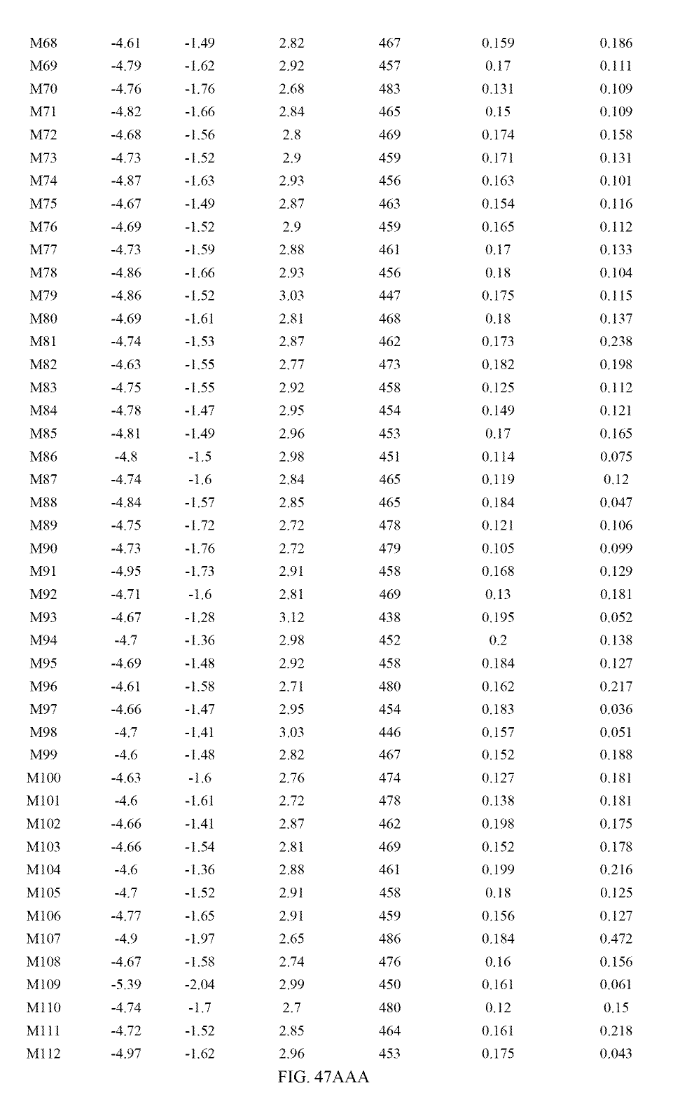

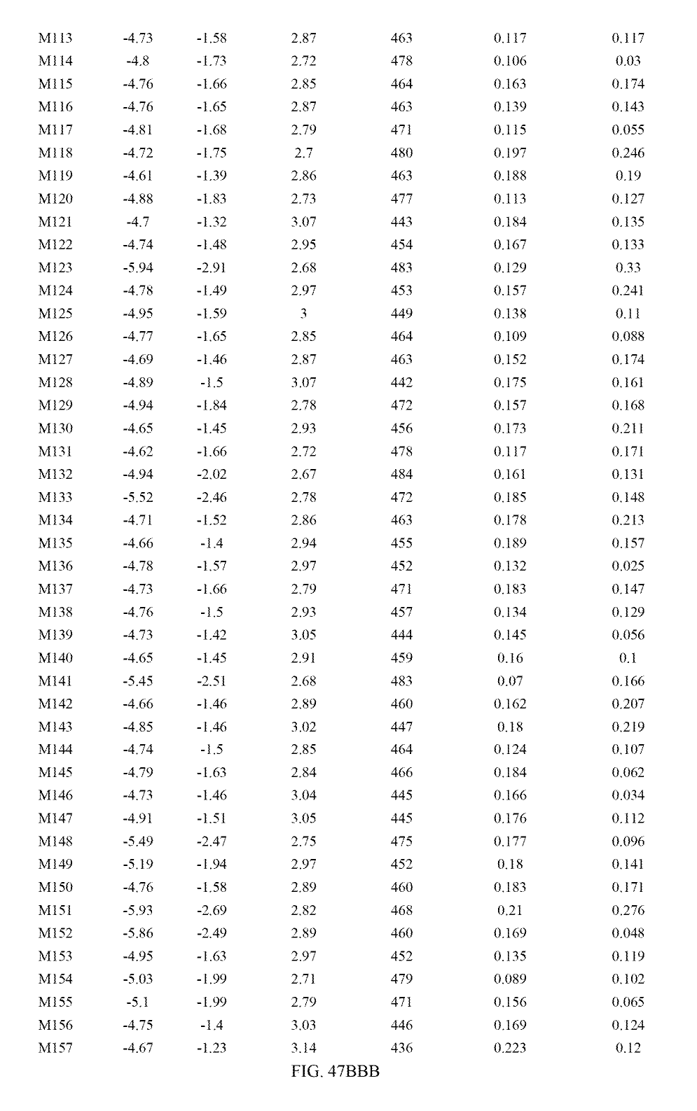

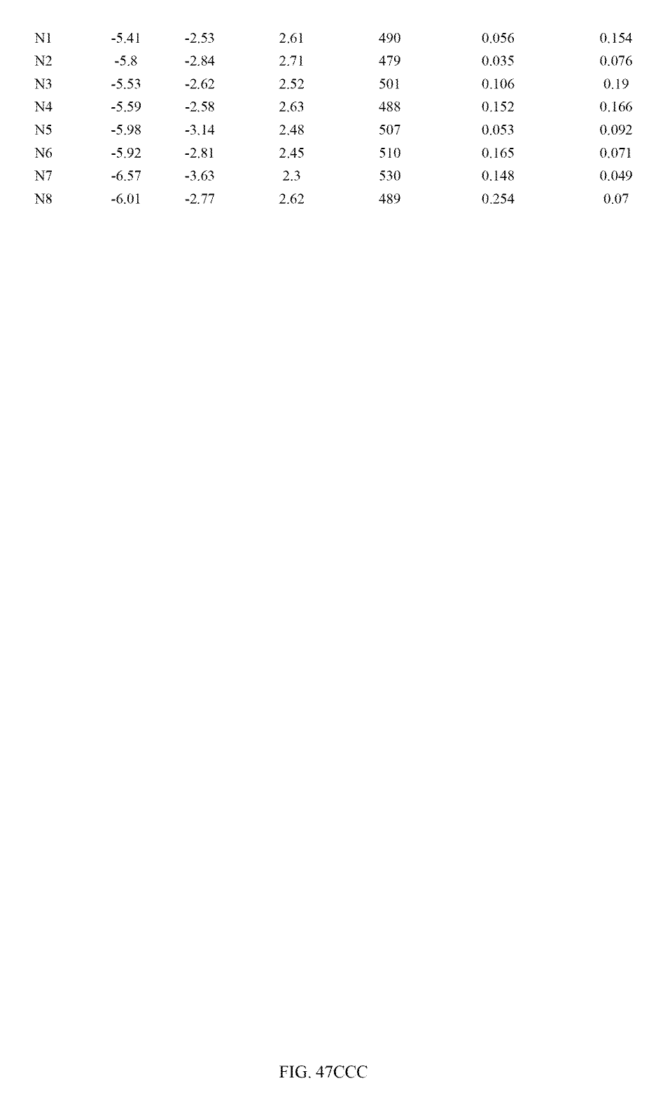

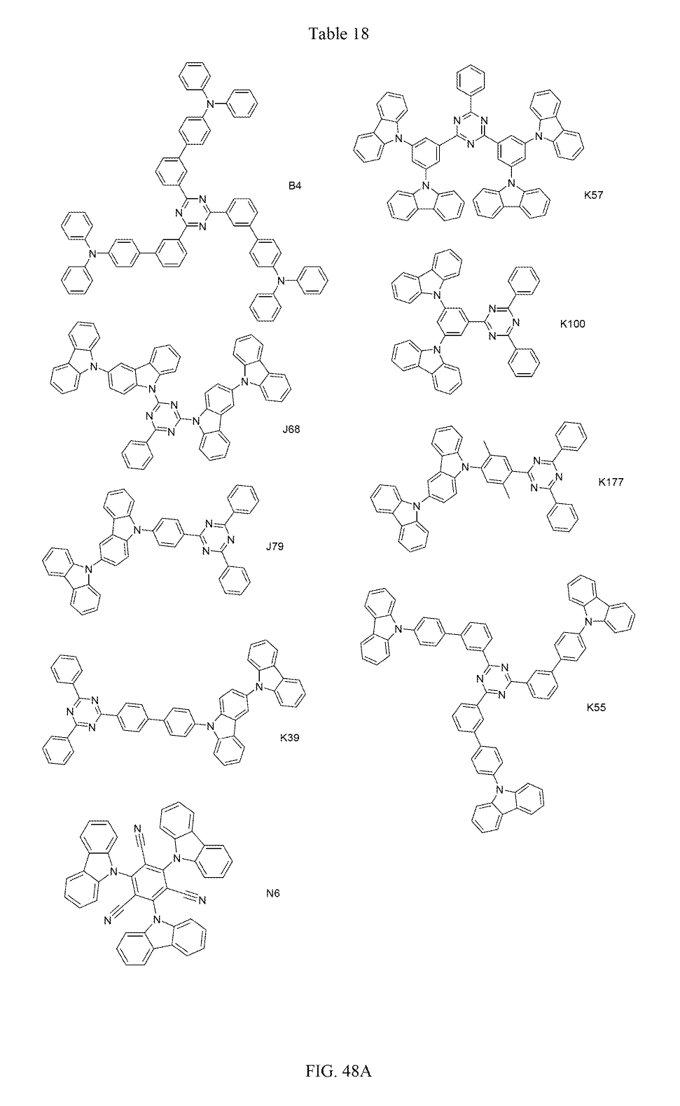

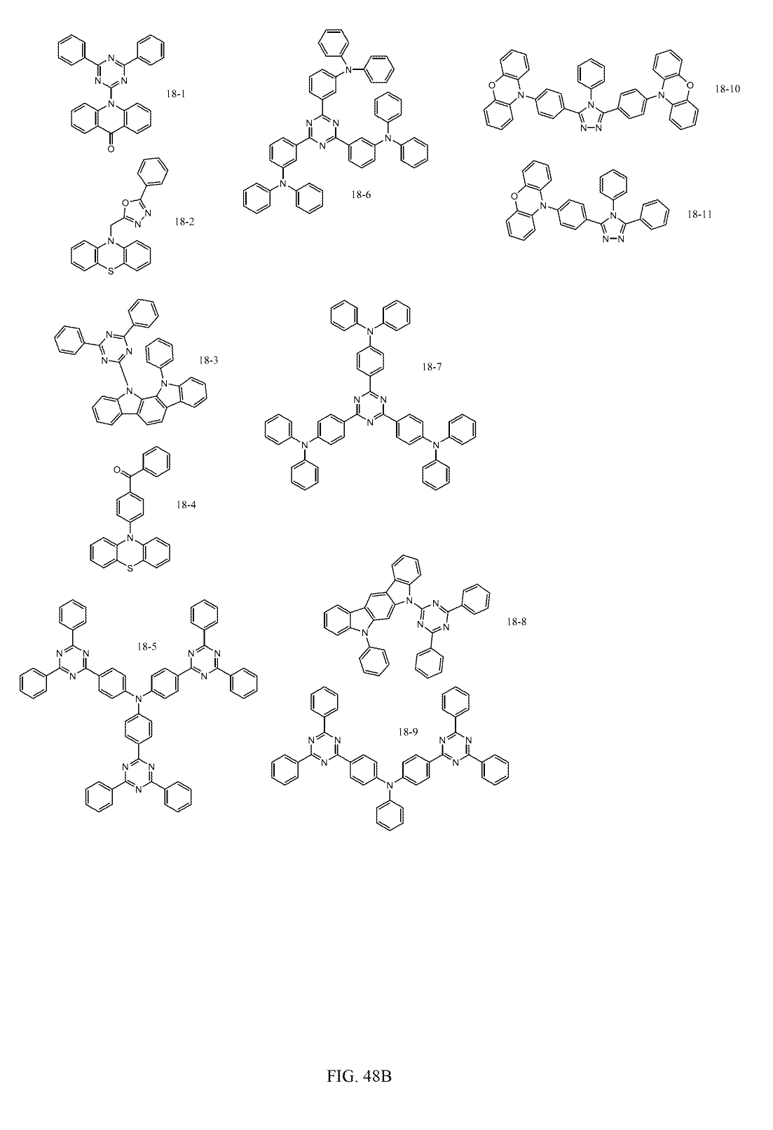

Compound S28-8 is the starting material for the reaction schemes described in FIGS. 30A-C. Compound S28-8 can be synthesized by a person of ordinary skill following Scheme 28 illustrated in FIG. 29. In the first step, compound S28-1 (available for purchase from Sigma-Aldrich Co., CAS No. 108-67-8) was combined with bromine and Fe in chloroform at room temperature. The reaction was allowed to stir for 24 hours to produce compound S28-2 in 90% yield. In the second step, compound 28-2 was combined with bromine in dichloroethane, heated to 100° C., and exposed to light. The reaction was allowed to stir for 12 hours to produce compound S28-3 in 95% yield. In the third step, compound S28-3 was combined with KOAc in acetic acid and heated to 140° C. The reaction was allowed to stir for 24 hours to produce compound S28-4. In the fourth step, compound S28-4 was combined with KOH in water and heated to 100° C. The reaction was allowed to stir for 12 h to form compound S28-5. In the fifth step, compound S28-5 was combined with KMnO₄ in water and heated to 100° C. The reaction was allowed to stir for 12 hours to form compound S28-6. In the sixth step, compound S28-6 was combined with SOCl₂ in THF to form compound S28-7 in 100% yield. In the seventh step, compound S28-7 was combined with NH₃.H₂O at 0° C. and allowed to stir for 6 hours to form S28-8. In the eighth step, compound S28-8 was combined with POCl₃ to form compound S28-9. It is understood that steps 1, 2, 3, 4, 5, 6, 7, and 8 can be performed and optimized by a person having ordinary skill in the art without undue experimentation.

### Compounds N1, N3, N4, and M141

Compounds N1-N8 and M141 can be synthesized by a person of ordinary skill following Scheme 29 illustrated in FIG. 30A. Starting material S28-9 is combined with S29-1, S29-2, S29-3, or S29-7 and Pd/K₂CO₃ in THF/H₂O and heated to 85° C. The reactions can be allowed to stir for 12 hours to produce compounds N1, N3, N4, and M141 respectively. It is understood that these steps can be performed and optimized by a person having ordinary skill in the art without undue experimentation.

### Compounds N6 and N8

Compounds N6 and N8 can be synthesized by a person of ordinary skill following Scheme 30 illustrated in FIG. 30B. Starting material S28-9 is combined with S29-4 or S29-6 and CuI/Cs₂CO₃ in DMF and heated to 100° C. The reactions can be allowed to stir for 12 hours to produce compounds N6 and N8, respectively. It is understood that these steps can be performed and optimized by a person having ordinary skill in the art without undue experimentation.

### Compound N7

Compound N7 can be synthesized by a person of ordinary skill following Scheme 31 illustrated in FIG. 30C. Starting material S28-9 is combined with S29-5 and TEA in 1,4-dioxane and heated to 80° C. The reaction can be allowed to stir for 24 hours to produce compound N7. It is understood that these steps can be performed and optimized by a person having ordinary skill in the art without undue experimentation.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A molecule represented by structural formula (XII): 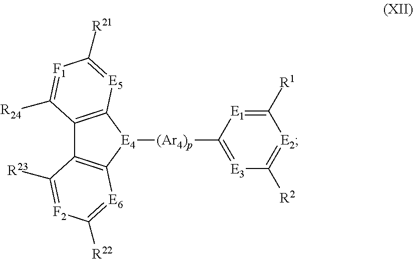
wherein:
E₁, E₂, E₃, E₄, E₅, and E₆, are, each independently, CR″ or N, wherein R″ is H or a C₁-C₃ alkyl;
R¹ and R² are, each independently, H, a C₁-C₆ alkyl, a C₆-C₁₈ aryl, or a (5-20) atom heteroaryl;
R²¹, R², R²³, and R²⁴ are, each independently, H, or a C₁-C₃ alkyl;
F₁ and F₂ are, each independently, CR′ or N, wherein R′ is H, a C₁-C₆ alkyl, a C₆-C₁₈ aryl, or —(Ar₅)_{q}-G;
Ar₄ and Ar₅ are, each independently, phenyl optionally substituted with one to four C₁-C₃ alkyls;
p is 0, 1, or 2;
q is 0 or 1; and
G is H, or a moiety represented by one of the following structural formula: 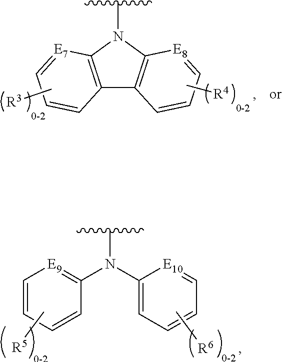
wherein:
E₇, E₈, E₉, and E₁₀ are, each independently, CH or N, and
R³, R⁴, R⁵, and R⁶ are, each independently, a C₁-C₃ alkyl, a C₆-C₁₈ aryl, a halo, or —CN,
with a proviso that when E₁, E₂, and E₃ are each N, and F₁ and F₂ are each CR′, then each R′ is not the moiety represented by the structural formula

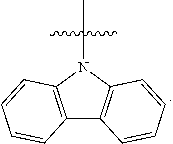 [SMILES: *n1c2ccccc2c2ccccc21]

2. The molecule of claim 1, wherein:
R¹ and R² are, each independently, H or C₆-C₁₂ aryl; and
p is 1.

3. The molecule of claim 1 or 2, wherein:
Ar₄ and Ar₅ are, each independently, a moiety represented by the following structural formula:

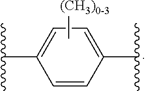

4. The molecule of any one of claims 1 to 3, wherein:
F₁ and F₂ are, each independently, a CR′.

5. The molecule of any one of claims 1 to 4, wherein q is 0.

6. The molecule of any one of claims 1 to 5, wherein G is H or is a moiety represented by the following structural formula:

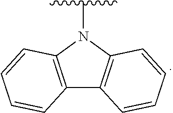 [SMILES: *n1c2ccccc2c2ccccc21]

7. The molecule of claim 1, represented by the following structural formula: 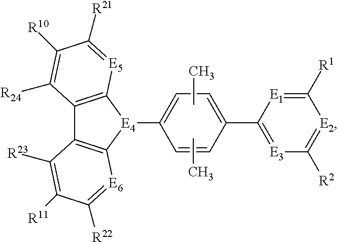
wherein:
R¹ and R² are, each independently, H or C₆-C₁₂ aryl; and
R¹⁰ and R¹¹ are, each independently, H or is a moiety represented by the following structural formula:

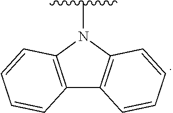 [SMILES: *n1c2ccccc2c2ccccc21]

8. The molecule of any one of claims 1 to 7, wherein the molecule is represented by the following structural formula:

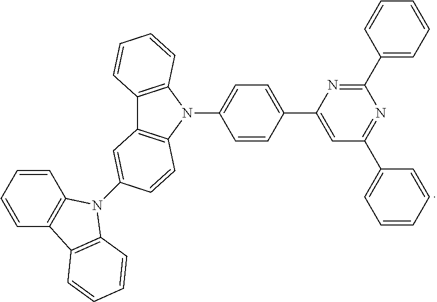 [SMILES: c1ccc(-c2cc(-c3ccc(-n4c5ccccc5c5cc(-n6c7ccccc7c7ccccc76)ccc54)cc3)nc(-c3ccccc3)n2)cc1]

9. The molecule of claims 1 to 7, wherein the molecule is represented by the following structural formula:

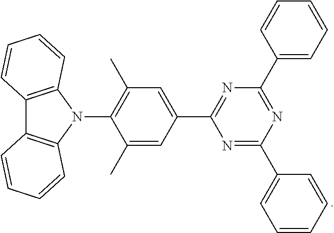 [SMILES: Cc1cc(-c2nc(-c3ccccc3)nc(-c3ccccc3)n2)cc(C)c1-n1c2ccccc2c2ccccc21]

10. A molecule comprising:
at least one moiety A;
at least one moiety D; and
optionally, one or more moiety B, wherein each moiety B is multivalent;
wherein moieties A are different from moieties D;
wherein each moiety A is covalently attached to either at least one of the moieties B or at least one moiety D;
wherein each moiety D is covalently attached to either at least one of the moieties B or at least one moiety A;
wherein each moiety B is covalently attached to at least one of the moieties A and at least one of the moieties D;
wherein the moiety A, for each occurrence independently, is selected from List A1, List A2, List A3, or any combination thereof;
wherein the moiety D, for each occurrence independently, is selected from List D1, List D2, List D3, or any combination thereof;
wherein each moiety B, for each occurrence independently, is selected List B1, List B2, or both; and
wherein the molecule is represented by any one of the structural formulas in Tables 1-14,
wherein the carbon or heteroatom denoted by (*) in the structural formulas represented in Tables 1-14 is unsubstituted or substituted by a C₁-C₆ alkyl, —OH, —CN, a halo, a C₆-C₁₂ aryl, a 5-20 atom heteroaryl, —N(R¹⁹)₂ or —N(R²⁰)₂,
wherein each R¹⁹, independently, is H, a C₁-C₆ alkyl, or a C₅-C₁₂ cycloalkyl, and wherein each R²⁰, independently, is H or a C₆-C₁₈ aryl, and
with the proviso that the molecule is not represented by the structural formulas B4, J68, J79, K39, K55, K57, K100, K177, or N6 in Tables 1-14.

11. The molecule of claim 10, wherein the molecule is represented by any one structural formula selected from Table 15.

12. A molecule represented by structural formulas (II)-(XI): 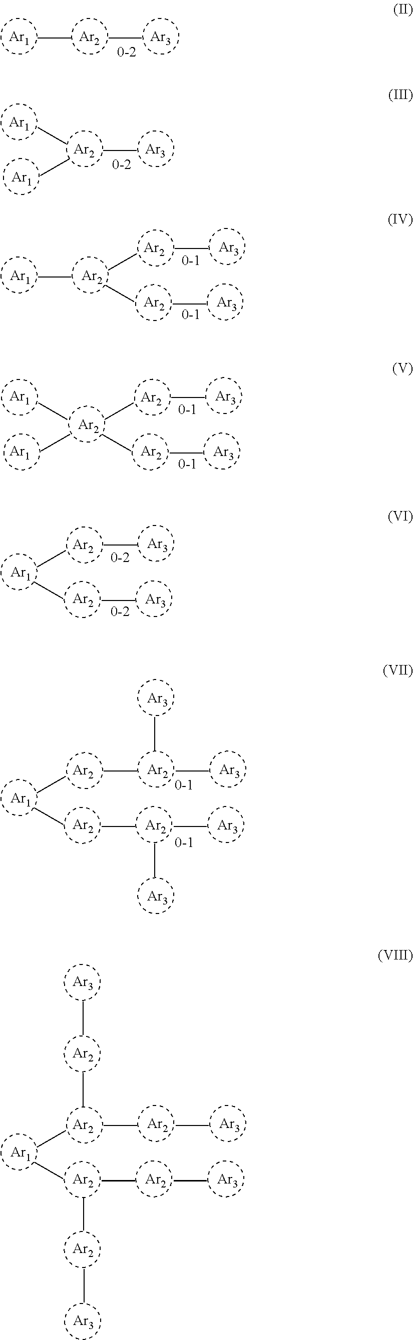 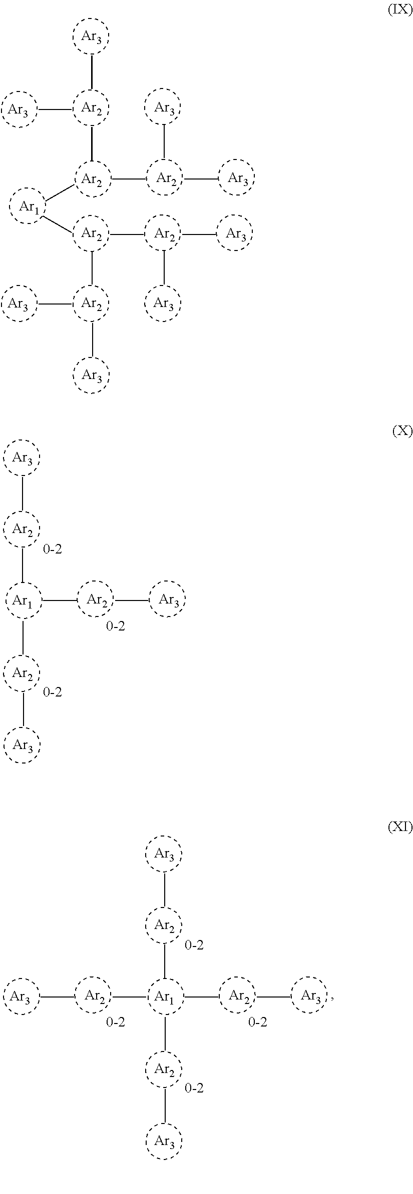
wherein:
Ar₁ and Ar₃, for each occurrence independently, are selected from List M1, with the understanding that Ar₁ and Ar₃ are different;
Ar₂ is, for each occurrence independently, selected List M2; and and further wherein the molecule is represented by any one of the structural formulas in Tables 1-14, wherein the carbon or heteroatom denoted by (*) in the structural formulas represented in Tables 1-14 is unsubstituted or substituted by a C₁-C₆ alkyl, —OH, —CN, a halo, a C₆-C₁₂ aryl, a 5-20 atom heteroaryl, —N(R¹⁹)₂, or —N(R²⁰)₂,
wherein each R¹⁹, independently, is H or a C₁-C₆ alkyl, or a C₅-C₁₂ cycloalkyl, and wherein each R²⁰, independently, is H or a C₆-C₁₈ aryl, and
with the proviso that the molecule is not represented by the structural formulas B4, J68, J79, K39, K55, K57, K100, K177, or N6 in Tables 1-14.

13. The molecule of claim 12, wherein the molecule is selected from Table 15.

14. The molecule of any one of claims 10 to 13, wherein the molecule is represented by any one of the following structural formulas:

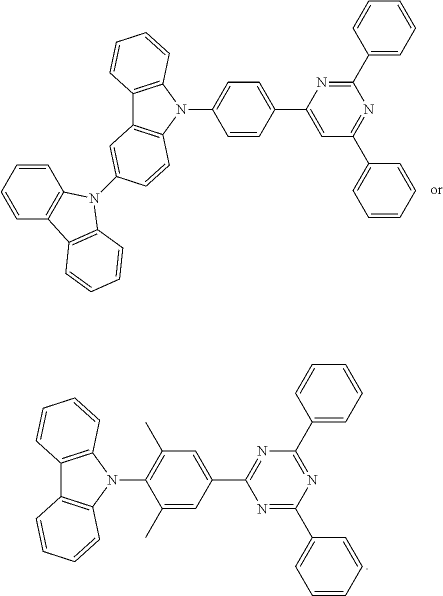 [SMILES: c1ccc(-c2cc(-c3ccc(-n4c5ccccc5c5cc(-n6c7ccccc7c7ccccc76)ccc54)cc3)nc(-c3ccccc3)n2)cc1 ; Cc1cc(-c2nc(-c3ccccc3)nc(-c3ccccc3)n2)cc(C)c1-n1c2ccccc2c2ccccc21]

15. An organic light-emitting device comprising:
a first electrode;
a second electrode; and
an organic layer disposed between the first electrode and the second electrode, and
wherein the organic layer comprises at least one molecule as defined by any one of claims 1-14.
